(19) United States
(12) Patent Application Publication
(10) Pub. No.: US 2009/0311217 A1
(43) Pub. Date: Dec. 17, 2009

(54) 3-SUBSTITUTED-1H-INDOLE COMPOUNDS, THEIR USE AS MTOR KINASE AND PI3 KINASE INHIBITORS, AND THEIR SYNTHESES

(75) Inventors: Matthew Gregory Bursavich, Salt Lake City, UT (US); Nan Zhang, Bayside, NY (US); Semiramis Ayral-Kaloustian, Tarrytown, NY (US); James Thomas Anderson, Hillsdale, NJ (US); Thai Hiep Nguyen, Fair Lawn, NJ (US); Sabrina Lombardi, New Haven, CT (US); David Malwitz, Hopewell Junction, NY (US); Natasja Brooijmans, New York, NY (US); Derek Cecil Cole, New City, NY (US); Adam Matthew Gilbert, Congers, NY (US); Pawel Wojciech Nowak, Woodcliff Lake, NJ (US); Kaapjoo Park, Congers, NY (US); Sasmita Das, West Nyack, NY (US); Hwei-Ru Tsou, New City, NY (US); Aranapakam Mudumbai Venkatesan, Rego Park, NY (US); Mercy Adufa Otteng, Pearl River, NY (US); Gary Harold Birnberg, Tuxedo Park, NY (US); Gloria Jean MacEwan, Monroe, NY (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/473,605

(22) Filed: May 28, 2009

Related U.S. Application Data
(60) Provisional application No. 61/056,655, filed on May 28, 2008.

(51) Int. Cl. A61K 31/404; C07D 405/02; C07D 403/14; C07D 413/02; A61K 31/496; A61K 31/5377; A61K 38/21; A61K 39/395; A61P 35/00

(52) U.S. Cl. 424/85.6; 548/454; 544/373; 544/121; 514/414; 514/254.09; 514/235.2; 424/133.1

(57) ABSTRACT

The invention relates to 3-substituted-1H-indole compounds of the Formula I:

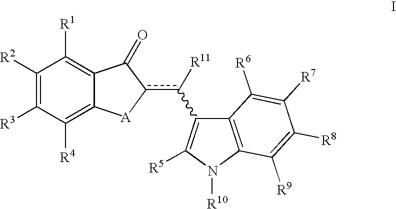

or a pharmaceutically acceptable salt thereof, wherein the constituent variables are as defined herein, compositions comprising the compounds, and methods for making and using the compounds.

### FIELD OF THE INVENTION

The invention relates to 3-substituted-1H-indole compounds, compositions comprising such compound, methods of synthesizing such compounds, and methods for treating mTOR-related diseases comprising the administration of an effective amount of such a compound. The invention also relates to methods for treating PI3K-related diseases comprising the administration of an effective amount of such a compound.

### BACKGROUND OF THE INVENTION

Phosphatidylinositol (hereinafter abbreviated as “PI”) is one of the phospholipids in cell membranes. In recent years it has become clear that PI plays an important role also in intracellular signal transduction. It is well recognized in the art that PI (4,5) bisphosphate (PI(4,5)P2 or PIP2) is degraded into diacylglycerol and inositol (1,4,5) triphosphate by phospholipase C to induce activation of protein kinase C and intracellular calcium mobilization, respectively [M. J. Berridge et al., Nature, 312, 315 (1984); Y. Nishizuka, Science, 225, 1365 (1984)].

In the late 1980s, phosphatidylinositol-3 kinase (“PI3K”) was found to be an enzyme that phosphorylates the 3-position of the inositol ring of phosphatidylinositol [D. Whitman et al., Nature, 332, 664 (1988)]. When PI3K was discovered, it was originally considered to be a single enzyme. Recently however, it was clarified that a plurality of PI3K subtypes exists. Three major subtypes of PI3Ks have now been identified on the basis of their in vitro substrate specificity, and these three are designated class I (a & b), class II, and class III [B. Vanhaesebroeck, Trend in Biol. Sci., 22, 267 (1997)].

The class Ia PI3K subtype has been most extensively investigated to date. Within the class Ia subtype there are three isoforms (α, β, & δ) that exist as hetero dimers of a catalytic 110-kDa subunit and regulatory subunits of 50-85 kDa. The regulatory subunits contain SH2 domains that bind to phosphorylated tyrosine residues within growth factor receptors or adaptor molecules and thereby localize PI3K to the inner cell membrane. At the inner cell membrane PI3K converts PIP2 to PIP3 (phosphatidylinositol-3,4,5-trisphosphate) that serves to localize the downstream effectors PDK1 and Akt to the inner cell membrane where Akt activation occurs. Activated Akt mediates a diverse array of effects including inhibition of apoptosis, cell cycle progression, response to insulin signaling, and cell proliferation. Class Ia PI3K subtypes also contain Ras binding domains (RBD) that allow association with activated Ras providing another mechanism for PI3K membrane localization. Activated, oncogenic forms of growth factor receptors, Ras, and even PI3K kinase have been shown to aberrantly elevate signaling in the PI3K/Akt/mTOR pathway resulting in cell transformation. As a central component of the PI3K/Akt/mTOR signaling pathway PI3K (particularly the class Ia α isoform) has become a major therapeutic target in cancer drug discovery.

Substrates for class I PI3Ks are PI, PI(4)P and PI(4,5)P2, with PI(4,5)P2 being the most favored. Class I PI3Ks are further divided into two groups, class Ia and class Ib, because of their activation mechanism and associated regulatory subunits. The class Ib PI3K is p110γ that is activated by interaction with G protein-coupled receptors. Interaction between p110γ and G protein-coupled receptors is mediated by regulatory subunits of 110, 87, and 84 kDa.

PI and PI(4)P are the known substrates for class II PI3Ks; PI(4,5)P2 is not a substrate for the enzymes of this class. Class II PI3Ks include PI3K C2α, C2β, and C2γ isoforms, which contain C2 domains at the C terminus, implying that their activity is regulated by calcium ions.

The substrate for class III PI3Ks is PI only. A mechanism for activation of the class III PI3Ks has not been clarified. Because each subtype has its own mechanism for regulating activity, it is likely that activation mechanism(s) depend on stimuli specific to each respective class of PI3K.

The compound PI103 (3-(4-(4-morpholinyl)pyrido[3′,2′:4,5]furo[3,2-d]pyrimidin-2-yl)phenol) inhibits PI3K_{α} and PI3K_{γ} as well as the mTOR complexes with IC₅₀ values of 2, 3, and 50-80 nM respectively. I.P. dosing in mice of this compound in human tumor xenograft models of cancer demonstrated activity against a number of human tumor models, including the glioblastoma (PTEN null U87MG), prostate (PC3), breast (MDA-MB-468 and MDA-MB-435) colon carcinoma (HCT 116); and ovarian carcinoma (SKOV3 and IGROV-1); (Raynaud et al, Pharmacologic Characterization of a Potent Inhibitor of Class I Phosphatidylinositide 3-Kinases, Cancer Res. 2007 67: 5840-5850).

The compound ZSTK474 (2-(2-difluoromethylbenzoimidazol-1-yl)-4,6-dimorpholino-1,3,5-triazine) inhibits PI3K_{α} and PI3K_{γ} but not the mTOR enzymes with IC₅₀ values of 16, 4.6 and >10,000 nM respectively (Dexin Kong and Takao Yamori, ZSTK474 is an ATP-competitive inhibitor of class I phosphatidylinositol 3 kinase isoforms, Cancer Science, 2007, 98:10 1638-1642). Chronic oral administration of ZSTK474 in mouse human xenograft cancer models, completely inhibited growth that originated from a non-small-cell lung cancer (A549), a prostate cancer (PC-3), and a colon cancer (WiDr) at a dose of 400 mg/kg. (Yaguchi et al, Antitumor Activity of ZSTK474, a New Phosphatidylinositol 3-Kinase Inhibitor, J. Natl. Cancer Inst. 98: 545-556).

The compound NVP-BEZ-235 (2-methyl-2-(4-(3-methyl-2-oxo-8-(quinolin-3-yl)-2,3-dihydro-1H-imidazo[4,5-c]quinolin-1-yl)phenyl)propanenitrile) inhibits both PI3K_{α} and PI3K_{γ} as well as the mTOR enzyme with IC₅₀ values 4, 5, and “nanomolar”. Testing in human tumor xenograft models of cancer demonstrated activity against human tumor models of prostrate (PC-3) and glioblastoma (U-87) cancer. It entered clinical trials in December of 2006 (Verheijen, J. C. and Zask, A., Phosphatidylinositol 3-kinase (PI3K) inhibitors as anticancer drugs, Drugs Fut 2007, 32(6): 537-547).

The compound SF-1126 (a prodrug form of LY-294002, which is 2-(4-morpholinyl)-8-phenyl-4H-1-benzopyran-4-one) is “a pan-PI3K inhibitor”. It is active in preclinical mouse cancer models of prostrate, breast, ovarian, lung, multiple myeloma, and brain cancers. It began clinical trials in April, 2007 for the solid tumors endometrial, renal cell, breast, hormone refractory prostate, and ovarian cancers. (Verheijen, J. C. and Zask, A., Phosphatidylinositol 3-kinase (PI3K) inhibitors as anticancer drugs, Drugs Fut. 2007, 32(6): 537-547).

Exelixis Inc. (So. San Francisco, Calif.) recently filed INDs for XL-147 (a selective pan-PI3K inhibitor of unknown structure) and XL-765 (a mixed inhibitor of mTOR and PI3K of unknown structure) as anticancer agents. TargeGen's short-acting mixed inhibitor of PI3Kγ and δ, TG-100115, is in phase I/II trials for treatment of infarct following myocardial ischemia-reperfusion injury. Cerylid's antithrombotic PI3Kβ inhibitor CBL-1309 (structure unknown) has completed preclinical toxicology studies.

According to Verheijen, J. C. and Zask, A., Phosphatidylinositol 3-kinase (PI3K) inhibitors as anticancer drugs, Drugs Fut. 2007, 32(6): 537-547,
Although it seems clear that inhibition of the α isoform is essential for the antitumor activity of PI3K inhibitors, it is not clear whether a more selective inhibitor of a particular PI3K isoform may lead to fewer unwanted biological effects. It has recently been reported that non-PI3Kα class I isoforms (PI3Kβ, δ and γ) have the ability to induce oncogenic transformation of cells, suggesting that nonisoform-specific inhibitors may offer enhanced therapeutic potential over specific inhibitors.
Selectivity versus other related kinases is also an important consideration for the development of PI3K inhibitors. While selective inhibitors may be preferred in order to avoid unwanted side effects, there have been reports that inhibition of multiple targets in the PI3K/Akt pathway (e.g., PI3Kα and mTOR [mammalian target of rapamycin]) may lead to greater efficacy. It is possible that lipid kinase inhibitors may parallel protein kinase inhibitors in that nonselective inhibitors may also be brought forward to the clinic.

Mammalian Target of Rapamycin, mTOR, is a cell-signaling protein that regulates the response of tumor cells to nutrients and growth factors, as well as controlling tumor blood supply through effects on Vascular Endothelial Growth Factor, VEGF. Inhibitors of mTOR starve cancer cells and shrink tumors by inhibiting the effect of mTOR. All mTOR inhibitors bind to the mTOR kinase. This has at least two important effects. First, mTOR is a downstream mediator of the PI3K/Akt pathway. The PI3K/Akt pathway is thought to be over-activated in numerous cancers and may account for the widespread response from various cancers to mTOR inhibitors. The over-activation of the upstream pathway would normally cause mTOR kinase to be over-activated as well. However, in the presence of mTOR inhibitors, this process is blocked. The blocking effect prevents mTOR from signaling to downstream pathways that control cell growth. Over-activation of the PI3K/Akt kinase pathway is frequently associated with mutations in the PTEN gene, which is common in many cancers and may help predict what tumors will respond to mTOR inhibitors. The second major effect of mTOR inhibition is anti-angiogenesis, via the lowering of VEGF levels.

In lab tests, certain chemotherapy agents were found to be more effective in the presence of mTOR inhibitors. George, J. N., et al., Cancer Research, 61, 1527-1532, 2001. Additional lab results have shown that some rhabdomyosarcoma cells die in the presence of mTOR inhibitors. The complete functions of the mTOR kinase and the effects of mTOR inhibition are not completely understood.

There are three mTOR inhibitors, which have progressed into clinical trials. These compounds are Wyeth's Torisel, also known as 42-(3-hydroxy-2-(hydroxymethyl)-rapamycin 2-methylpropanoate, CCI-779 or Temsirolimus; Novartis' Everolimus, also known as 42-O-(2-hydroxyethyl)-rapamycin, or RAD 001; and Ariad's AP23573 also known as 42-(dimethylphopsinoyl)-rapamycin. The FDA has approved Torisel for the treatment of advanced renal cell carcinoma. In addition, Torisel is active in a NOS/SCID xenograft mouse model of acute lymphoblastic leukemia [Teachey et al, Blood, 107(3), 1149-1155, 2006]. On Mar. 30, 2009, the Food and Drug Administration (FDA) approved Everolimus (AFINITOR™) for the treatment of patients with advanced renal cell carcinoma. AP23573 has been given orphan drug and fast-track status by the FDA for treatment of soft-tissue and bone sarcomas.

The three mTOR inhibitors have non-linear, although reproducible pharmacokinetic profiles. Mean area under the curve (AUC) values for these drugs increase at a less than dose related way. The three compounds are all semi-synthetic derivatives of the natural macrolide antibiotic rapamycin. It would be desirable to find fully synthetic compounds, which inhibit mTOR that are more potent and exhibit improved pharmacokinetic behaviors.

As explained above, PI3K inhibitors and mTOR inhibitors are expected to be novel types of medicaments useful against cell proliferation disorders, especially as carcinostatic agents. Thus, it would be advantageous to have new PI3K inhibitors and mTOR inhibitors as potential treatment regimens for mTOR- and PI3K-related diseases. The instant invention is directed to these and other important ends.

### SUMMARY OF THE INVENTION

In one aspect, the invention provides compounds of the Formula I:

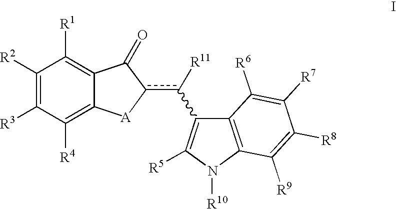

or a pharmaceutically acceptable salt thereof, wherein the constituent variables are as defined below. In other aspects, the invention provides compositions comprising a compound of the invention, and methods for making compounds of the invention. In further aspects, the invention provides methods for inhibiting PI3K, mTOR, and hSMG-1 in a subject, and methods for treating PI3K-related, mTOR-related, and hSMG-1-related disorders in a mammal in need thereof.

### DETAILED DESCRIPTION OF THE INVENTION

In one aspect, the invention provides compounds of the Formula: I:

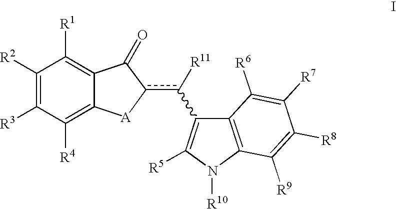

or a geometric isomer thereof or a pharmaceutically acceptable salt thereof wherein

A is oxygen or sulfur;

[symbol] (dashed line) represents an optional second carbon-to-carbon bond;

R¹, R², R³, and R⁴ are each independently H; C₁-C₆alkoxy optionally substituted with from 1 to 3 substituents independently selected from H₂N—, C₁-C₆aminoalkyl-, and di(C₁-C₆alkyl)amino-; C₁-C₆alkyl; (C₁-C₆alkoxy)carbonyl; C₆-C₁₄aryl optionally substituted with from 1 to 3 substituents independently selected from R¹²C(O)NH—; R¹⁴OC(O)NR¹²—, H₂N—, C₁-C₆aminoalkyl-, and di(C₁-C₆alkyl)amino-; C₁-C₉heteroaryl optionally substituted with from 1 to 3 substituents independently selected from R¹²C(O)NH—, R¹⁴OC(O)NR¹²—, H₂N—, C₁-C₆aminoalkyl-, and di(C₁-C₆alkyl)amino-; HO₂C—; C₁-C₆hydroxylalkyl-; R¹²R¹³N—; R¹²R¹³NC(O)—; C₁-C₉heterocyclyl-C(O)—; R¹²R¹³NC(O)NH—; R¹²R¹³NC(S)NH—; R¹²R¹³NC(O)O—; C₁-C₉heterocyclyl-C(O)NH—; R¹²C(O)NH—; R¹⁴OC(O)NR¹²—; R¹⁴OC(O)NHC(O)NH—; R¹²S—; R¹²S(O)—; R¹²S(O)₂—; R¹²S(O)₂—O—; R¹²C(O)—; R¹²S(O)₂—NR¹²—; R¹²R¹³NS(O)₂—; C₂-C₆alkenyl; C₂-C₆alkynyl; C₁-C₉heterocyclyl-optionally substituted by C₁-C₆alkyl; halo; CN; NO₂; or hydroxyl;

R¹² and R¹³ are each independently: a) H; b) C₁-C₆alkyl optionally substituted with from 1 to 3 substituents independently selected from: i) H₂N—, ii) C₁-C₆aminoalkyl-, iii) di(C₁-C₆alkyl)amino-, iv) C₁-C₉heteroaryl, v) halo, vi) hydroxyl, vii) C₁-C₆alkoxy optionally substituted with from 1 to 3 substituents independently selected from: A) hydroxyl, B) C₁-C₆alkoxy, C) H₂N—, D) C₁-C₆aminoalkyl-, and E) di(C₁-C₆alkyl)amino-, viii) C₁-C₉heterocyclyl, ix) di(C₁-C₆alkyl)amino-optionally substituted with from 1 to 3 substituents independently selected from: A) hydroxyl, B) C₁-C₆alkoxy, C) H₂N—, D) C₁-C₆aminoalkyl-, and E) di(C₁-C₆alkyl)amino-; c) C₂-C₆alkenyl; d) C₂-C₆alkynyl; e) C₁-C₉heterocyclyl-optionally substituted by C₁-C₆alkyl; f) perfluoro(C₁-C₆)alkyl; g) C₁-C₉heteroaryl optionally substituted with from 1 to 3 substituents independently selected from: i) C₁-C₆alkyl optionally substituted with a substituent selected from: A) hydroxyl, B) H₂N—, C) C₁-C₆aminoalkyl-, D) di(C₁-C₆alkyl)amino-, and E) C₁-C₉heterocyclyl-optionally substituted by C₁-C₆alkyl, ii) halo, iii) hydroxyl, iv) C₁-C₆alkoxy, v) H₂N—, vi) C₁-C₆aminoalkyl-, vii) di(C₁-C₆alkyl)amino-, viii) O₂N—, ix) H₂NSO₂—, x) HO₂C—, xi) (C₁-C₆alkoxy)carbonyl, xii) (C₁-C₆alkoxy)carbonyl-NH—, xiii) Q-Z-, wherein Z is A) —O—, B) —N(CH₃)—, C) —NH—, D) —C(O)N(CH₃)—, E) —C(O)NH—, F) —N(CH₃)C(O)—, G) —NHC(O)—, H) —NHSO₂—, I) —N(CH₃)SO₂— J) —SO₂NH—, K) —SO₂N(CH₃)—, L) —NHC(O)NH—, M) —S—, N) —S(O)—, O) S(O)₂, or P) is absent, and Q is selected from: A) C₆-C₁₄aryl, B) C₁-C₉heteroaryl, C) C₁-C₉heterocyclyl-optionally substituted with from 1 to 3 substituents independently selected from: 1) C₁-C₆alkyl, 2) C₁-C₆hydroxylalkyl-, 3) di(C₁-C₆alkyl)amino-, and 4) perfluoro(C₁-C₆)alkyl; D) C₃-C₈cycloalkyl, E) C₁-C₆alkyl, F) C₂-C₆alkenyl, G) C₂-C₆alkynyl, H) (C₁-C₆alkyl)amino-C₁-C₆alkylene-, I) di(C₁-C₆alkyl)amino-C₁-C₆alkylene-, J) (C₆-C₁₄aryl)alkyl, K) (C₁-C₉heteroaryl)alkyl, or L) heterocyclyl(C₁-C₆alkyl), xiv) HC(O)—, xv) (C₁-C₆alkyl)C(O)—, xvi) (C₃-C₈cycloalkyl)C(O)—, xvii) (C₁-C₉heterocyclyl)C(O)— optionally substituted with A) C₁-C₆alkyl, B) C₁-C₆hydroxylalkyl-, C) di(C₁-C₆alkyl)amino-, or D) perfluoro(C₁-C₆)alkyl; h) C₆-C₁₄aryl optionally substituted with from 1 to 3 substituents independently selected from: i) C₁-C₆alkyl optionally substituted with a substituent selected from: A) hydroxyl, B) H₂N—, C) C₁-C₆aminoalkyl-, D) di(C₁-C₆alkyl)amino-, and E) C₁-C₉heterocyclyl-optionally substituted by C₁-C₆alkyl, ii) halo, iii) hydroxyl, iv) C₁-C₆alkoxy, v) H₂N—, vi) C₁-C₆aminoalkyl-, vii) di(C₁-C₆alkyl)amino-, viii) O₂N—, ix) H₂NSO₂—, x) HO₂C—, xi) (C₁-C₆alkoxy)carbonyl, xii) (C₁-C₆alkoxy)carbonyl-NH—, xiii) Q-Z-, wherein Z is A) —O—, B) —N(CH₃)—, C) —NH—, D) —C(O)N(CH₃)—, E) —C(O)NH—, F) —N(CH₃)C(O)—, G) —NHC(O)—, H) —NHSO₂—, I) —N(CH₃)SO₂— J) —SO₂NH—, K) —SO₂N(CH₃)—, L) —NHC(O)NH—, M) —S—, N) —S(O)—, O) S(O)₂, or P) is absent, and Q is selected from: A) C₆-C₁₄aryl, B) C₁-C₉heteroaryl, C) C₁-C₉heterocyclyl-optionally substituted with from 1 to 3 substituents independently selected from: 1) C₁-C₆alkyl, 2) C₁-C₆hydroxylalkyl-, 3) di(C₁-C₆alkyl)amino-, and 4) perfluoro(C₁-C₆)alkyl; D) C₃-C₈cycloalkyl, E) C₁-C₆alkyl, F) C₂-C₆alkenyl, G) C₂-C₆alkynyl, H) (C₁-C₆alkyl)amino-C₁-C₆alkylene-, I) di(C₁-C₆alkyl)amino-C₁-C₆alkylene-, J) (C₆-C₁₄aryl)alkyl, K) (C₁-C₉heteroaryl)alkyl, or L) heterocyclyl(C₁-C₆alkyl), xiv) HC(O)—, xv) (C₁-C₆alkyl)C(O)—, xvi) (C₃-C₈cycloalkyl)C(O)—, xvii) (C₁-C₉heterocyclyl)C(O)— optionally substituted with A) C₁-C₆alkyl, B) C₁-C₆hydroxylalkyl-, C) di(C₁-C₆alkyl)amino-, or D) perfluoro(C₁-C₆)alkyl; or i) C₃-C₈cycloalkyl;

R¹⁴ is independently C₁-C₆alkyl, C₁-C₆hydroxylalkyl-, or C₆-C₁₄aryl;

R⁵ is H; C₁-C₆alkyl optionally substituted with from 1 to 3 substituents independently selected from halo, H₂N—, C₁-C₆aminoalkyl-, di(C₁-C₆alkyl)amino-, (CH₃)₂N(CH₂)₂N(CH₃)—, —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆alkoxy, C₁-C₆alkyl, —C(O)OH, (C₁-C₆alkoxy)carbonyl, —C(O)(C₁-C₆alkyl), C₆-C₁₄aryl, C₁-C₉heteroaryl, C₃-C₈cycloalkyl, C₁-C₆haloalkyl-, C₁-C₆aminoalkyl-, —OC(O)(C₁-C₆alkyl), C₁-C₆carboxyamidoalkyl-, and —NO₂; C₆-C₁₄aryl optionally substituted with from 1 to 3 substituents independently selected from C₁-C₆alkoxy, C₁-C₆alkyl, (C₆-C₁₄aryl)alkyl-O—, C₃-C₈cycloalkyl, di(C₁-C₆alkyl)amino-C₁-C₆alkylene-, C₁-C₆ perfluoroalkyl-, halo, C₁-C₆haloalkyl-, hydroxyl, C₁-C₆hydroxylalkyl-, H₂N—, C₁-C₆aminoalkyl-, di(C₁-C₆alkyl)amino-, —COOH, —C(O)O—(C₁-C₆alkyl), —OC(O)(C₁-C₆alkyl), (C₁-C₆alkyl)carboxyamido, —C(O)NH₂, (C₁-C₆alkyl)amido-, —O—CH₂CH₂OCH₃, —O—CH₂CH₂OCH₂CH₃, —O—CH₂CH₂OCH₂CH₂OCH₃, —O—CH₂CH₂OCH₂CH₂OCH₂CH₃, and —NO₂; C₃-C₈cycloalkyl; halo; C₁-C₉heteroaryl optionally substituted with from 1 to 3 substituents independently selected from C₁-C₆alkoxy, C₁-C₆alkyl, C₃-C₈cycloalkyl, di(C₁-C₆alkyl)amino-C₁-C₆alkylene-, C₁-C₆ perfluoroalkyl-, halo, C₁-C₆haloalkyl-, hydroxyl, C₁-C₆hydroxylalkyl-, H₂N—, C₁-C₆aminoalkyl-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, (C₁-C₆alkyl)amido-, —O—CH₂CH₂OCH₃, —O—CH₂CH₂OCH₂CH₃, —O—CH₂CH₂OCH₂CH₂OCH₃, —O—CH₂CH₂OCH₂CH₂OCH₂CH₃, and —NO₂; C₁-C₉heterocyclyl-optionally substituted by C₁-C₆alkyl; C₁-C₆heterocyclylalkyl optionally substituted with from 1 to 3 C₁-C₆alkyl groups; C₁-C₆ perfluoroalkyl-; R¹⁵R¹⁶NC(O)—; CN; (C₁-C₆alkoxy)carbonyl; or CO₂H;

R¹⁵ and R¹⁶ are each independently H; C₁-C₆alkyl optionally substituted with from 1 to 3 substituents independently selected from hydroxyl, H₂N—, —NH(C₁-C₆alkyl), —N(C₁-C₆alkyl)(C₁-C₆alkyl), and C₁-C₉heteroaryl; C₁-C₉heteroaryl; C₆-C₁₄aryl optionally substituted with from 1 to 3 substituents independently selected from C₁-C₆alkyl, halo, and perfluoro(C₁-C₆)alkyl; C₃-C₈cycloalkyl;

or R¹⁵ and R¹⁶, when taken together with the nitrogen to which they are attached, form a 3- to 7-membered heterocycle, which heterocycle may optionally comprise 1 or 2 additional heteroatoms independently selected from —N(H)—, —N(C₁-C₆alkyl)-, —N(C₆-C₁₄aryl)-, —S—, —SO—, —S(O)₂, and —O—;

R⁶, R⁷, R⁸, and R⁹ are independently selected from:

a) H; b) C₁-C₆alkoxy optionally substituted by C₁-C₆alkoxy; c) C₁-C₆alkyl optionally substituted with from 1 to 3 substituents independently selected from: i) C₆-C₁₄aryl; ii) H₂N—, iii) C₁-C₆aminoalkyl-, iv) di(C₁-C₆alkyl)amino-, and v) C₁-C₉heterocyclyl optionally substituted by C₁-C₆alkyl; d) C₂-C₆alkenyl optionally substituted with from 1 to 3 substituents independently selected from: i) C₆-C₁₄aryl; ii) H₂N—, iii) C₁-C₆aminoalkyl-, iv) di(C₁-C₆alkyl)amino-, and v) C₁-C₉heterocyclyl optionally substituted by C₁-C₆alkyl; e) C₂-C₆alkynyl optionally substituted with from 1 to 3 substituents independently selected from: i) C₆-C₁₄aryl; ii) H₂N—, iii) C₁-C₆aminoalkyl-, iv) di(C₁-C₆alkyl)amino-, and v) C₁-C₉heterocyclyl optionally substituted by C₁-C₆alkyl; f) (C₁-C₆alkyl)amido-; g) C₁-C₆alkylcarboxy; h) (C₁-C₆alkyl)carboxyamido; i) (C₁-C₆alkyl)SO₂—; j) C₆-C₁₄aryl optionally substituted with from 1 to 3 substituents independently selected from: i) C₁-C₈acyl, ii) C₁-C₆alkyl, which is optionally substituted with from 1 to 3 substituents independently selected from: A) H₂N—, B) C₁-C₆aminoalkyl-, C) di(C₁-C₆alkyl)amino-, and D) C₁-C₉heterocyclyl-optionally substituted by C₁-C₆alkyl, iii) (C₁-C₆alkyl)amido-, iv) (C₁-C₆alkyl)carboxyl, v) (C₁-C₆alkyl)carboxyamido, vi) C₁-C₆alkoxy optionally substituted by C₁-C₆alkoxy or C₁-C₉heteroaryl, vii) (C₁-C₆alkoxy)carbonyl, viii) (C₆-C₁₄aryl)oxy, ix) C₃-C₈cycloalkyl, x) halo, xi) C₁-C₆haloalkyl-, xii) C₁-C₉heterocyclyl optionally substituted by C₁-C₆alkyl or C₁-C₆hydroxylalkyl-, xiii) hydroxyl, xiv) C₁-C₆hydroxylalkyl-, xv) C₁-C₆ perfluoroalkyl-, xvi) C₁-C₆ perfluoroalkyl-O—, xvii) R¹⁷R¹⁸N—, xviii) C₁-C₉heterocyclyl-optionally substituted by C₁-C₆alkyl, xix) CN, xx) —COOH, xxi) R¹⁷R¹⁸NC(O)—, xxii) C₁-C₉heterocyclyl-C(O)—, xxiii) R¹⁷C(O)NH—, xxiv) R¹⁷R¹⁸NS(O)₂—, xxv) C₁-C₉heterocyclyl-S(O)₂—, xxvi) R¹⁷R¹⁸NC(O)NH—, xxvii) C₁-C₉heterocyclyl-C(O)NH—, xxviii) R¹⁹OC(O)NH—, xxix) (C₁-C₆alkyl)S(O)₂NH—, xxx) R¹⁹S(O)₂—, xxxi) —C(═N—(OR¹⁷))—(NR¹⁷R¹⁸), and xxxii) —NO₂; k) (C₆-C₁₄aryl)alkyl-O—; I) (C₆-C₁₄aryl)oxy; m) halogen; n) C₁-C₉heteroaryl optionally substituted with from 1 to 3 substituents independently selected from: i) C₁-C₈acyl, ii) C₁-C₆alkyl, which is optionally substituted with from 1 to 3 substituents independently selected from: A) H₂N—, B) C₁-C₆aminoalkyl-, C) di(C₁-C₆alkyl)amino-, and D) C₁-C₉heterocyclyl-optionally substituted by C₁-C₆alkyl, iii) (C₁-C₆alkyl)amido-, iv) (C₁-C₆alkyl)carboxyl, v) (C₁-C₆alkyl)carboxyamido, vi) C₁-C₆alkoxy optionally substituted by C₁-C₆alkoxy or C₁-C₉heteroaryl, vii) (C₁-C₆alkoxy)carbonyl, viii) (C₆-C₁₄aryl)oxy, ix) C₃-C₈cycloalkyl, x) halo, xi) C₁-C₆haloalkyl-, xii) C₁-C₉heterocyclyl optionally substituted by C₁-C₆alkyl or C₁-C₆hydroxylalkyl-, xiii) hydroxyl, xiv) C₁-C₆hydroxylalkyl-, xv) C₁-C₆ perfluoroalkyl-, xvi) C₁-C₆ perfluoroalkyl-O—, xvii) R¹⁷R¹⁸N—, xviii) C₁-C₉heterocyclyl-optionally substituted by C₁-C₆alkyl, xix) CN, xx) —COOH, xxi) R¹⁷R¹⁸NC(O)—, xxii) C₁-C₉heterocyclyl-C(O)—, xxiii) R¹⁷C(O)NH—, xxiv) R¹⁷R¹⁸NS(O)₂—, xxv) C₁-C₉heterocyclyl-S(O)₂—, xxvi) R¹⁷R¹⁸NC(O)NH—, xxvii) C₁-C₉heterocyclyl-C(O)NH—, xxviii) R¹⁹OC(O)NH—, xxix) (C₁-C₆alkyl)S(O)₂NH—, xxx) R¹⁹S(O)₂—, xxxi) —C(═N—(OR¹⁷))—(NR¹⁷R¹⁸), and xxxii) —NO₂; o) hydroxyl; p) H₂N—; q) R¹⁷C(O)NH—; r) C₁-C₆alkylS(O)₂—O— s) C₁-C₉heterocyclyl optionally substituted with from 1 to 3 substituents independently selected from: i) C₁-C₆alkyl, which is optionally substituted with from 1 to 3 substituents independently selected from: A) H₂N—, B) C₁-C₆aminoalkyl-, and C) di(C₁-C₆alkyl)amino-, ii) R¹⁷R¹⁸NC(O)—, iii) hydroxyl, and iv) R¹⁷R¹⁸N—; t) C₁-C₆ perfluoroalkyl-; u) CN; v) (C₁-C₆alkoxy)carbonyl; w) CO₂H; and x) NO₂;

or R⁷ and R⁸ when taken together can be replaced by an alkylenedioxy group so that the alkylenedioxy group, when taken together with the two carbon atoms to which it is attached, forms a 5- to 7-membered heterocycle containing two oxygen atoms;

R¹⁷ and R¹³ are each independently H; C₁-C₆alkyl optionally substituted with from 1 to 3 substituents independently selected from C₁-C₆alkoxy, H₂N—, C₁-C₆aminoalkyl-, di(C₁-C₆alkyl)amino-, C₆-C₁₄aryl, C₁-C₉heterocyclyl-optionally substituted by C₁-C₆alkyl, and C₁-C₉heteroaryl; C₁-C₆alkoxy; C₁-C₉heteroaryl; hydroxyl; C₆-C₁₄aryl optionally substituted with from 1 to 3 substituents independently selected from C₁-C₆alkyl, halo, and perfluoro(C₁-C₆)alkyl; and C₃-C₈cycloalkyl;

or R¹⁷ and R¹⁸ when taken together with the nitrogen to which they are attached form a 3- to 7-membered heterocycle, which heterocycle may optionally comprise 1 or 2 additional heteroatoms independently selected from —N(H)—, —N(C₁-C₆alkyl)-, —N(C₆-C₁₄aryl)-, —S—, —SO—, —S(O)₂, or —O—; —N(H)—, —N(C₁-C₆alkyl)-, —N(C₁-C₆hydroxylalkyl)-, —N(C₁-C₆alkylene-di(C₁-C₆alkyl)amino)-, —N(C₆-C₁₄aryl)-, —S—, —SO—, —S(O)₂, and —O—;

R¹⁹ is C₁-C₆alkyl or C₆-C₁₄aryl;

R¹⁰ is C₁-C₆alkyl substituted with from 1 to 3 substituents independently selected from halogen, hydroxyl, C₁-C₆hydroxylalkyl-NH—, C₁-C₆hydroxylalkyl-N(CH₃)—, H₂N—, C₁-C₆aminoalkyl-, di(C₁-C₆alkyl)amino-, di(C₁-C₆alkyl)amino-(C₁-C₆alkylene)-NH—, di(C₁-C₆alkyl)amino-(C₁-C₆alkylene)-N(CH₃)—, —N(C₁-C₃alkyl)C(O)(C₁-C₆alkyl), —NHC(O)(C₁-C₆alkyl), —C(O)N(C₁-C₆alkyl)(C₁-C₆alkyl), —CN, C₁-C₆alkoxy, C₃-C₈cycloalkyl, C₁-C₆haloalkyl-, C₁-C₆aminoalkyl-, —OC(O)(C₁-C₆alkyl), —C(O)OH, (C₁-C₆alkoxy)carbonyl, —C(O)(C₁-C₆alkyl), —NHC(O)H, —C(O)NH₂, and —NO₂; C₂-C₁₀alkenyl; C₆-C₁₄aryl; (C₆-C₁₄aryl)alkyl; C₃-C₈cycloalkyl; C₁-C₉heteroaryl; (C₁-C₉heteroaryl)alkyl; C₁-C₆carboxyamidoalkyl-; or C₁-C₆heterocyclylalkyl group optionally substituted with from 1 to 3 substituents independently selected from halogen, H₂N—, C₁-C₆aminoalkyl-, di(C₁-C₆alkyl)amino-, —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆hydroxylalkyl-, C₁-C₆alkoxy, C₁-C₆alkyl, —C(O)OH, (C₁-C₆alkoxy)carbonyl, —C(O)(C₁-C₆alkyl), 4- to 7-membered monocyclic heterocycle, C₆-C₁₄aryl C₁-C₉heteroaryl, C₁-C₆heterocyclylalkyl, and C₃-C₈cycloalkyl;

or R¹⁰ is H, C₁-C₆alkyl, or C₁-C₈acyl provided that:

1) R² is not hydrogen, or 2) R³ is not hydroxyl, C₁-C₆alkoxy, or (C₁-C₆alkoxy)carbonyl, or 3) R⁵ is not H, C₁-C₆alkyl, or C₃-C₈cycloalkyl, or 4) any of R⁶, R⁷ R⁸ or R⁹ is: a) C₁-C₆alkoxy substituted by C₁-C₆alkoxy; b) C₁-C₆alkyl optionally substituted by C₆-C₁₄aryl; c) (C₁-C₆alkyl)SO₂—; d) C₆-C₁₄aryl optionally substituted with from 1 to 3 substituents independently selected from: i) C₁-C₈acyl, ii) C₁-C₆alkyl, which is optionally substituted with from 1 to 3 substituents independently selected from: A) H₂N—, C₁-C₆aminoalkyl-, B) di(C₁-C₆alkyl)amino-, and C) C₁-C₉heterocyclyl-optionally substituted by C₁-C₆alkyl, iii) (C₁-C₆alkyl)amido-, iv) (C₁-C₆alkyl)carboxyl, v) (C₁-C₆alkyl)carboxyamido, vi) C₁-C₆alkoxy optionally substituted by C₁-C₆alkoxy or C₁-C₉heteroaryl, vii) vii) (C₁-C₆alkoxy)carbonyl, viii) (C₆-C₁₄aryl)oxy, ix) C₃-C₈cycloalkyl, x) halo, xi) C₁-C₆haloalkyl-, xii)) C₁-C₉heterocyclyl optionally substituted by C₁-C₆alkyl or C₁-C₆hydroxylalkyl-, xiii) hydroxyl, xiv) C₁-C₆hydroxylalkyl-, xv) C₁-C₆ perfluoroalkyl-, xvi) C₁-C₆ perfluoroalkyl-O—, xvii) R¹⁷R¹¹N—, xviii) C₁-C₉heterocyclyl-optionally substituted by C₁-C₆alkyl, xix) CN, xx) —COOH, xxi) R¹⁷R¹⁸NC(O)—, xxii) C₁-C₉heterocyclyl-C(O)—, xxiii) R¹⁷C(O)NH—, xxiv) R¹⁷R¹⁸NS(O)₂—, xxv) C₁-C₉heterocyclyl-S(O)₂—, xxvi) R¹⁷R¹⁸NC(O)NH—, xxvii) C₁-C₉heterocyclyl-C(O)NH—, xxviii) R¹⁹OC(O)NH—, xxix (C₁-C₆alkyl)S(O)₂NH—, xxx) R¹⁹S(O)₂—, xxxi) —C(═N—(OR¹⁷))—(NR¹⁷R¹⁸), and xxxi) —NO₂; e) (C₆-C₁₄aryl)alkyl-O—; f) halo; C₁-C₉heteroaryl optionally substituted with from 1 to 3 substituents independently selected from: i) C₁-C₈acyl, ii) C₁-C₆alkyl, which is optionally substituted with from 1 to 3 substituents independently selected from: A) H₂N—, C₁-C₆aminoalkyl-, B) di(C₁-C₆alkyl)amino-, and C) C₁-C₉heterocyclyl-optionally substituted by C₁-C₆alkyl, iii) (C₁-C₆alkyl)amido-, iv) (C₁-C₆alkyl)carboxyl, v) (C₁-C₆alkyl)carboxyamido, vi) C₁-C₆alkoxy optionally substituted by C₁-C₆alkoxy or C₁-C₉heteroaryl, vii) vii) (C₁-C₆alkoxy)carbonyl, viii) (C₆-C₁₄aryl)oxy, ix) C₃-C₈cycloalkyl, x) halo, xi) C₁-C₆haloalkyl-, xii)) C₁-C₉heterocyclyl optionally substituted by C₁-C₆alkyl or C₁-C₆hydroxylalkyl-, xiii) hydroxyl, xiv) C₁-C₆hydroxylalkyl-, xv) C₁-C₆ perfluoroalkyl-, xvi) C₁-C₆ perfluoroalkyl-O—, xvii) R¹⁷R¹⁸N—, xviii) C₁-C₉heterocyclyl-optionally substituted by C₁-C₆alkyl, xix) CN, xx) —COOH, xxi) R¹⁷R¹⁸NC(O)—, xxii) C₁-C₉heterocyclyl-C(O)—, xxiii) R¹⁷C(O)NH—, xxiv) R¹⁷R¹⁸NS(O)₂—, xxv) C₁-C₉heterocyclyl-S(O)₂—, xxvi) R¹⁷R¹⁸NC(O)NH—, xxvii) C₁-C₉heterocyclyl-C(O)NH—, xxviii) R¹⁹OC(O)NH—, xxix (C₁-C₆alkyl)S(O)₂NH—, xxx) R¹⁹S(O)₂—, xxxi) —C(═N—(OR¹⁷))—(NR¹⁷R¹⁸), and xxxi) —NO₂; h) hydroxyl; i) C₁-C₉heterocyclyl optionally substituted with from 1 to 3 substituents independently selected from: i) C₁-C₆alkyl, which is optionally substituted with from 1 to 3 substituents independently selected from: A) H₂N—, B) C₁-C₆aminoalkyl-, and C) di(C₁-C₆alkyl)amino-, ii) R¹⁷R¹⁸NC(O)—, iii) hydroxyl, and iv) R¹⁷R¹⁸N—; j) C₁-C₆ perfluoroalkyl-; k) CN; I) (C₁-C₆alkoxy)carbonyl; m) CO₂H; and n) NO₂; or 5) any of R⁶, R⁸ or R⁹ is C₁-C₆alkoxy;

R¹¹ is H or C₁-C₆alkyl;

with the proviso (1) that R¹, R², R³, R⁴, R⁶, R⁷, R⁸, and R⁹ cannot simultaneously be H; and (2) that 4-hydroxy-6-methyl-2-[(1-methyl-1H-indol-3-yl)methylene]-(2H)-benzofuranone, 2-[(5-bromo-1H-indol-3-yl)methylene]-benzo[b]thiophen-3(2H)-one, (2Z)-5-chloro-2-[(2-phenyl-1H-indol-3-yl)methylene]-1-benzothiophen-3(2H)-one, and 2-[(7-ethyl-1H-indol-3-yl)methylene]-benzo[b]thiophen-3(2H)-one are excluded.

In one embodiment, A is oxygen.

In one embodiment, R¹ is H.

In one embodiment, R² is R¹²R¹³NC(O)NH—.

In one embodiment, R¹² is C₆-C₁₄aryl substituted with di(C₁-C₆alkyl)amino-C₂-C₆alkylene-N(C₁-C₆alkyl)C(O)—.

In one embodiment, R³ is H.

In one embodiment, R⁴ is H.

In one embodiment, R⁵ is C₁-C₉heteroaryl independently substituted with from 1 to 3 C₁-C₆alkyl substituents.

In one embodiment, R⁵ is 1,3,5-trimethyl-1H-pyrazol-4-yl.

In one embodiment, R⁶ is H.

In one embodiment, R⁷ is C₁-C₆alkoxy.

In one embodiment, R⁷ is CH₃O—.

In one embodiment, R⁸ is H.

In one embodiment, R⁹ is halogen.

In one embodiment, R¹⁰ is H.

In one embodiment, R¹¹ is H.

In one embodiment, R¹ is H or hydroxyl.

In one embodiment, R² is H or R¹²R¹³NC(O)NH—.

In one embodiment, R³ is H or hydroxyl.

In one embodiment, R⁵ is H, C₁-C₆alkyl, C₃-C₈cycloalkyl, C₁-C₉heteroaryl, optionally independently substituted with from 1 to 3 substituents as specified in Formula I, or R¹⁵R¹⁶NC(O)—.

In one embodiment, R⁶ is C₆-C₁₄aryl, C₁-C₉heterocyclyl-optionally substituted by C₁-C₆alkyl, C₁-C₉heteroaryl, each optionally independently substituted with from 1 to 3 substituents as specified in Formula I, or H.

In one embodiment, R⁷ is H or C₁-C₆alkoxy.

In one embodiment, R⁹ is H.

In one embodiment, R¹⁰ is H, C₁-C₆alkyl, or C₁-C₆heterocyclylalkyl group optionally substituted with from 1 to 3 substituents as specified in Formula I.

In one embodiment, R¹⁰ is C₁-C₆heterocyclylalkyl group optionally substituted with 1 C₁-C₆alkyl.

In one embodiment, R¹⁰ is (4-methylpiperazin-1-yl)ethyl.

In one embodiment, R¹⁰ is C₁-C₆alkyl.

In one embodiment, R¹⁰ is CH₃.

In one embodiment, R¹=R⁴=H.

In one embodiment, R⁶=R⁸=R⁹=H and R⁷ is C₁-C₆alkoxy.

In one embodiment, R⁶=R⁸=R⁹=H and R⁷ is CH₃O—.

In one embodiment, R⁷ is CH₃O— and R¹=R⁴=R⁶=R³=R⁹=R¹¹=H.

In one embodiment, R⁷ is CH₃O—, R¹=R⁴=R⁶=R³=R⁹=R¹¹=H, and R¹⁰ is (4-methylpiperazin-1-yl)ethyl.

In one embodiment, R¹=R³=hydroxyl.

In one embodiment, R²=R⁴=H.

In one embodiment, R⁵=R⁷=R⁸=R⁹=H.

In one embodiment, R⁶ is C₆-C₁₄aryl, C₁-C₉heterocyclyl-optionally substituted by C₁-C₆alkyl, C₁-C₉heteroaryl, each optionally independently substituted with from 1 to 3 substituents as specified in Formula I, and R¹⁰ is C₁-C₆alkyl.

In one embodiment, R⁶ is C₆-C₁₄aryl, C₁-C₉heterocyclyl-optionally substituted by C₁-C₆alkyl, C₁-C₉heteroaryl, each optionally independently substituted with from 1 to 3 substituents as specified in Formula I, and R¹⁰ is CH₃—.

In one embodiment, R⁶ is C₆-C₁₄aryl, C₁-C₉heterocyclyl-optionally substituted by C₁-C₆alkyl, C₁-C₉heteroaryl, each optionally independently substituted with from 1 to 3 substituents as specified in Formula I, R¹⁰ is CH₃—, R²=R⁴=R⁵=R⁷=R⁸=R⁹=R¹¹=H, and R¹=R³ hydroxyl.

Illustrative compounds of the present invention are set forth below:
2-(1H-indol-3-ylmethylene)-1-benzofuran-3(2H)-one;
2-[(2-phenyl-1H-indol-3-yl)methylene]-1-benzofuran-3(2H)-one;
2-[(1-methyl-2-phenyl-1H-indol-3-yl)methylene]-1-benzofuran-3(2H)-one;
2-[(1-methyl-1H-indol-3-yl)methylene]-1-benzofuran-3(2H)-one;
6-hydroxy-2-[(2-phenyl-1H-indol-3-yl)methylene]-1-benzofuran-3(2H)-one;
6-hydroxy-2-[(1-methyl-2-phenyl-1H-indol-3-yl)methylene]-1-benzofuran-3(2H)-one;
2-(1H-indol-3-ylmethylene)-7-methoxy-1-benzofuran-3(2H)-one;
7-methoxy-2-[(2-phenyl-1H-indol-3-yl)methylene]-1-benzofuran-3(2H)-one;
7-methoxy-2-[(1-methyl-2-phenyl-1H-indol-3-yl)methylene]-1-benzofuran-3(2H)-one;
7-methoxy-2-[(1-methyl-1H-indol-3-yl)methylene]-1-benzofuran-3(2H)-one;
4,6-dihydroxy-2-[(2-phenyl-1H-indol-3-yl)methylene]-1-benzofuran-3(2H)-one;
4,6-dihydroxy-2-[(1-methyl-2-phenyl-1H-indol-3-yl)methylene]-1-benzofuran-3(2H)-one;
(2Z)-2-{[2-(4-chlorophenyl)-1H-indol-3-yl]methylene}-1-benzofuran-3(2H)-one;
(2Z)-2-{[2-(2-naphthyl)-1H-indol-3-yl]methylene}-1-benzofuran-3(2H)-one;
(2Z)-2-{[2-(4-fluorophenyl)-1H-indol-3-yl]methylene}-1-benzofuran-3(2H)-one;
(2Z)-2-[(2-methyl-5-nitro-1H-indol-3-yl)methylene]-1-benzofuran-3(2H)-one;
(2Z)-2-[(2-methyl-1H-indol-3-yl)methylene]-1-benzofuran-3(2H)-one;
(2Z)-2-[(6-methyl-1H-indol-3-yl)methylene]-1-benzofuran-3(2H)-one;
(2Z)-2-[(7-methyl-1H-indol-3-yl)methylene]-1-benzofuran-3(2H)-one;
(2Z)-2-[(5-bromo-1H-indol-3-yl)methylene]-1-benzofuran-3(2H)-one;
(2Z)-2-[(1-benzyl-1H-indol-3-yl)methylene]-1-benzofuran-3(2H)-one;
(2Z)-2-{[2-(4-chlorophenyl)-1H-indol-3-yl]methylene}-6-hydroxy-1-benzofuran-3(2H)-one;
(2Z)-6-hydroxy-2-{[2-(2-naphthyl)-1H-indol-3-yl]methylene}-1-benzofuran-3(2H)-one;
(2Z)-2-{[2-(4-fluorophenyl)-1H-indol-3-yl]methylene}-6-hydroxy-1-benzofuran-3(2H)-one;
(2Z)-6-hydroxy-2-[(2-methyl-5-nitro-1H-indol-3-yl)methylene]-1-benzofuran-3(2H)-one;
(2Z)-6-hydroxy-2-[(6-methyl-1H-indol-3-yl)methylene]-1-benzofuran-3(2H)-one;
(2Z)-2-[(5-bromo-1H-indol-3-yl)methylene]-6-hydroxy-1-benzofuran-3(2H)-one;
(2Z)-2-[(1-benzyl-1H-indol-3-yl)methylene]-6-hydroxy-1-benzofuran-3(2H)-one;
(2Z)-2-{[5-(benzyloxy)-1H-indol-3-yl]methylene}-6-hydroxy-1-benzofuran-3(2H)-one;
(2Z)-2-{[2-(4-chlorophenyl)-1H-indol-3-yl]methylene}-7-methoxy-1-benzofuran-3(2H)-one;
(2Z)-7-methoxy-2-{[2-(2-naphthyl)-1H-indol-3-yl]methylene}-1-benzofuran-3(2H)-one;
(2Z)-2-{[2-(4-fluorophenyl)-1H-indol-3-yl]methylene}-7-methoxy-1-benzofuran-3(2H)-one;
(2Z)-7-methoxy-2-[(2-methyl-5-nitro-1H-indol-3-yl)methylene]-1-benzofuran-3(2H)-one;
(2Z)-7-methoxy-2-[(2-methyl-1H-indol-3-yl)methylene]-1-benzofuran-3(2H)-one;
(2Z)-7-methoxy-2-[(6-methyl-1H-indol-3-yl)methylene]-1-benzofuran-3(2H)-one;
(2Z)-2-[(5-bromo-1H-indol-3-yl)methylene]-7-methoxy-1-benzofuran-3(2H)-one;
(2Z)-2-[(1-benzyl-1H-indol-3-yl)methylene]-7-methoxy-1-benzofuran-3(2H)-one;
(2Z)-2-{[5-(benzyloxy)-1H-indol-3-yl]methylene}-7-methoxy-1-benzofuran-3(2H)-one;
(2Z)-2-{[2-(4-chlorophenyl)-1H-indol-3-yl]methylene}-4,6-dihydroxy-1-benzofuran-3(2H)-one;
(2Z)-4,6-dihydroxy-2-{[2-(2-naphthyl)-1H-indol-3-yl]methylene}-1-benzofuran-3(2H)-one;
(2Z)-2-{[2-(4-fluorophenyl)-1H-indol-3-yl]methylene}-4,6-dihydroxy-1-benzofuran-3(2H)-one;
(2Z)-4,6-dihydroxy-2-[(6-methyl-1H-indol-3-yl)methylene]-1-benzofuran-3(2H)-one;
(2Z)-4,6-dihydroxy-2-[(7-methyl-1H-indol-3-yl)methylene]-1-benzofuran-3(2H)-one;
(2Z)-2-[(5-bromo-1H-indol-3-yl)methylene]-4,6-dihydroxy-1-benzofuran-3(2H)-one;
(2Z)-2-[(1-benzyl-1H-indol-3-yl)methylene]-4,6-dihydroxy-1-benzofuran-3(2H)-one;
2-{[5-(benzyloxy)-1H-indol-3-yl]methylene}-4,6-dihydroxy-1-benzofuran-3(2H)-one;
(2Z)-6,7-dihydroxy-2-[(2-phenyl-1H-indol-3-yl)methylene]-1-benzofuran-3(2H)-one;
(2Z)-2-{[2-(4-chlorophenyl)-1H-indol-3-yl]methylene}-6,7-dihydroxy-1-benzofuran-3(2H)-one;
(2Z)-6,7-dihydroxy-2-{[2-(2-naphthyl)-1H-indol-3-yl]methylene}-1-benzofuran-3(2H)-one;
(2Z)-2-{[2-(4-fluorophenyl)-1H-indol-3-yl]methylene}-6,7-dihydroxy-1-benzofuran-3(2H)-one;
(2Z)-2-[(5-bromo-1H-indol-3-yl)methylene]-6,7-dihydroxy-1-benzofuran-3(2H)-one;
(2Z)-2-[(5-chloro-1H-indol-3-yl)methylene]-4,6-dihydroxy-1-benzofuran-3(2H)-one;
(2Z)-2-[(5-bromo-2-methyl-1H-indol-3-yl)methylene]-4,6-dihydroxy-1-benzofuran-3(2H)-one;
(2Z)-2-[(5-chloro-1H-indol-3-yl)methylene]-6,7-dihydroxy-1-benzofuran-3(2H)-one;
(2Z)-2-[(5-fluoro-1H-indol-3-yl)methylene]-6,7-dihydroxy-1-benzofuran-3(2H)-one;
(2Z)-6,7-dihydroxy-2-[(5-methyl-1H-indol-3-yl)methylene]-1-benzofuran-3(2H)-one;
(2Z)-2-[(5-bromo-2-methyl-1H-indol-3-yl)methylene]-6,7-dihydroxy-1-benzofuran-3(2H)-one;
(2Z)-7-hydroxy-2-[(2-phenyl-1H-indol-3-yl)methylene]-1-benzofuran-3(2H)-one;
(2Z)-2-{[2-(4-fluorophenyl)-1H-indol-3-yl]methylene}-7-hydroxy-1-benzofuran-3(2H)-one;
4,6-dihydroxy-2-[(5-methoxy-2-phenyl-1H-indol-3-yl)methylene]-1-benzofuran-3(2H)-one;
6,7-dihydroxy-2-[(5-methoxy-2-phenyl-1H-indol-3-yl)methylene]-1-benzofuran-3(2H)-one;
4,6-dihydroxy-2-[(2-pyridin-2-yl-1H-indol-3-yl)methylene]-1-benzofuran-3(2H)-one;
4-hydroxy-2-(1H-indol-3-ylmethylene)-1-benzofuran-3(2H)-one;
4-hydroxy-2-[(2-phenyl-1H-indol-3-yl)methylene]-1-benzofuran-3(2H)-one;
2-{[2-(4-chlorophenyl)-1H-indol-3-yl]methylene}-4-hydroxy-1-benzofuran-3(2H)-one;
2-[(5-bromo-1H-indol-3-yl)methylene]-4-hydroxy-1-benzofuran-3(2H)-one;
4-hydroxy-2-[(5-methoxy-2-phenyl-1H-indol-3-yl)methylene]-1-benzofuran-3(2H)-one;
2-[(2-bromo-1H-indol-3-yl)methylene]-4,6-dihydroxy-1-benzofuran-3(2H)-one;
2-[(2-bromo-1H-indol-3-yl)methylene]-6,7-dihydroxy-1-benzofuran-3(2H)-one;
4-hydroxy-2-[(5-methoxy-2-methyl-1H-indol-3-yl)methylene]-1-benzofuran-3(2H)-one;
2-{[1-(4-chlorobutyl)-5-methoxy-2-methyl-1H-indol-3-yl]methylene}-4,6-dihydroxy-1-benzofuran-3(2H)-one;
2-{[1-(3-chloropropyl)-5-methoxy-2-methyl-1H-indol-3-yl]methylene}-4,6-dihydroxy-1-benzofuran-3(2H)-one;
4,6-dihydroxy-2-[(5-methoxy-2-pyridin-3-yl-1H-indol-3-yl)methylene]-1-benzofuran-3(2H)-one;
2-{[1-(2-chloroethyl)-2-methyl-1H-indol-3-yl]methylene}-4,6-dihydroxy-1-benzofuran-3(2H)-one;
2-{[1-(3-chloropropyl)-2-methyl-1H-indol-3-yl]methylene}-4,6-dihydroxy-1-benzofuran-3(2H)-one;
2-{[1-(4-chlorobutyl)-2-methyl-1H-indol-3-yl]methylene}-4,6-dihydroxy-1-benzofuran-3(2H)-one;
4,6-dihydroxy-2-({5-methoxy-2-methyl-1-[3-(4-methylpiperazin-1-yl)propyl]-1H-indol-3-yl}methylene)-1-benzofuran-3(2H)-one;
4,6-dihydroxy-2-({5-methoxy-2-methyl-1-[4-(4-methylpiperazin-1-yl)butyl]-1H-indol-3-yl}methylene)-1-benzofuran-3(2H)-one;
4,6-dihydroxy-2-{[5-methoxy-2-methyl-1-(4-morpholin-4-ylbutyl)-1H-indol-3-yl]methylene}-1-benzofuran-3(2H)-one;
4,6-dihydroxy-2-[(1-{4-[4-(2-hydroxyethyl)piperazin-1-yl]butyl}-5-methoxy-2-methyl-1H-indol-3-yl)methylene]-1-benzofuran-3(2H)-one;
2-[(1-{4-[3-(dimethylamino)pyrrolidin-1-yl]butyl}-5-methoxy-2-methyl-1H-indol-3-yl)methylene]-4,6-dihydroxy-1-benzofuran-3(2H)-one;
4,6-dihydroxy-2-[(5-methoxy-2-methyl-1-{4-[4-(2-morpholin-4-ylethyl)piperazin-1-yl]butyl}-1H-indol-3-yl)methylene]-1-benzofuran-3(2H)-one;
2-({1-[4-(dimethylamino)butyl]-5-methoxy-2-methyl-1H-indol-3-yl}methylene)-4,6-dihydroxy-1-benzofuran-3(2H)-one;
4,6-dihydroxy-2-{[5-methoxy-2-methyl-1-(3-morpholin-4-ylpropyl)-1H-indol-3-yl]methylene}-1-benzofuran-3(2H)-one;
4,6-dihydroxy-2-[(1-{3-[4-(2-hydroxyethyl)piperazin-1-yl]propyl}-5-methoxy-2-methyl-1H-indol-3-yl)methylene]-1-benzofuran-3(2H)-one;
2-[(1-{3-[3-(dimethylamino)pyrrolidin-1-yl]propyl}-5-methoxy-2-methyl-1H-indol-3-yl)methylene]-4,6-dihydroxy-1-benzofuran-3(2H)-one;
4,6-dihydroxy-2-[(5-methoxy-2-methyl-1-{3-[4-(2-morpholin-4-ylethyl)piperazin-1-yl]propyl}-1H-indol-3-yl)methylene]-1-benzofuran-3(2H)-one;
2-({1-[3-(dimethylamino)propyl]-5-methoxy-2-methyl-1H-indol-3-yl}methylene)-4,6-dihydroxy-1-benzofuran-3(2H)-one;
4,6-dihydroxy-2-{[5-methoxy-2-methyl-1-(2-morpholin-4-ylethyl)-1H-indol-3-yl]methylene}-1-benzofuran-3(2H)-one;
4,6-dihydroxy-2-({5-methoxy-2-methyl-1-[2-(4-methylpiperazin-1-yl)ethyl]-1H-indol-3-yl}methylene)-1-benzofuran-3(2H)-one;
4,6-dihydroxy-2-[(1-{2-[4-(2-hydroxyethyl)piperazin-1-yl]ethyl}-5-methoxy-2-methyl-1H-indol-3-yl)methylene]-1-benzofuran-3(2H)-one;
2-({1-[2-(dimethylamino)ethyl]-5-methoxy-2-methyl-1H-indol-3-yl}methylene)-4,6-dihydroxy-1-benzofuran-3(2H)-one;
4,6-dihydroxy-2-[(1-{2-[4-(2-hydroxyethyl)piperazin-1-yl]ethyl}-5-methoxy-1H-indol-3-yl)methylene]-1-benzofuran-3(2H)-one;
4,6-dihydroxy-2-{[5-methoxy-1-(3-morpholin-4-ylpropyl)-1H-indol-3-yl]methylene}-1-benzofuran-3(2H)-one;
4,6-dihydroxy-2-[(1-{3-[4-(2-hydroxyethyl)piperazin-1-yl]propyl}-5-methoxy-1H-indol-3-yl)methylene]-1-benzofuran-3(2H)-one;
2-({1-[3-(dimethylamino)propyl]-5-methoxy-1H-indol-3-yl}methylene)-4,6-dihydroxy-1-benzofuran-3(2H)-one;
4,6-dihydroxy-2-{[5-methoxy-1-(4-morpholin-4-ylbutyl)-1H-indol-3-yl]methylene}-1-benzofuran-3(2H)-one;
2-({1-[4-(dimethylamino)butyl]-5-methoxy-1H-indol-3-yl}methylene)-4,6-dihydroxy-1-benzofuran-3(2H)-one;
4,6-dihydroxy-2-[(5-methoxy-1H-indol-3-yl)methyl]-1-benzofuran-3(2H)-one;
4,6-dihydroxy-2-[(5-methoxy-2-phenyl-1H-indol-3-yl)methyl]-1-benzofuran-3(2H)-one;
4,6-dihydroxy-2-[(5-methoxy-2-methyl-1H-indol-3-yl)methyl]-1-benzofuran-3(2H)-one;
7-hydroxy-2-[(5-methoxy-2-methyl-1H-indol-3-yl)methylene]-1-benzofuran-3(2H)-one;
4-hydroxy-2-[(5-methoxy-1,2-dimethyl-1H-indol-3-yl)methylene]-1-benzofuran-3(2H)-one;
2-{[1-(4-chlorobutyl)-5-methoxy-2-methyl-1H-indol-3-yl]methylene}-6,7-dihydroxy-1-benzofuran-3(2H)-one;
2-{[1-(4-chlorobutyl)-5-methoxy-2-methyl-1H-indol-3-yl]methylene}-4-hydroxy-1-benzofuran-3(2H)-one;
2-{[1-(3-chloropropyl)-5-methoxy-2-methyl-1H-indol-3-yl]methylene}-6,7-dihydroxy-1-benzofuran-3(2H)-one;
2-{[1-(3-chloropropyl)-5-methoxy-2-methyl-1H-indol-3-yl]methylene}-4-hydroxy-1-benzofuran-3(2H)-one;
4-hydroxy-2-({5-methoxy-2-methyl-1-[2-(4-methylpiperazin-1-yl)ethyl]-1H-indol-3-yl}methylene)-1-benzofuran-3(2H)-one;
4-hydroxy-2-{[5-methoxy-2-methyl-1-(2-morpholin-4-ylethyl)-1H-indol-3-yl]methylene}-1-benzofuran-3(2H)-one;
4-hydroxy-2-[(1-{2-[4-(2-hydroxyethyl)piperazin-1-yl]ethyl}-5-methoxy-2-methyl-1H-indol-3-yl)methylene]-1-benzofuran-3(2H)-one;
6,7-dihydroxy-2-({5-methoxy-2-methyl-1-[4-(4-methylpiperazin-1-yl)butyl]-1H-indol-3-yl}methylene)-1-benzofuran-3(2H)-one;
6,7-dihydroxy-2-{[5-methoxy-2-methyl-1-(4-morpholin-4-ylbutyl)-1H-indol-3-yl]methylene}-1-benzofuran-3(2H)-one;
6,7-dihydroxy-2-[(1-{4-[4-(2-hydroxyethyl)piperazin-1-yl]butyl}-5-methoxy-2-methyl-1H-indol-3-yl)methylene]-1-benzofuran-3(2H)-one;
4-hydroxy-2-({5-methoxy-2-methyl-1-[4-(4-methylpiperazin-1-yl)butyl]-1H-indol-3-yl}methylene)-1-benzofuran-3(2H)-one;
4-hydroxy-2-{[5-methoxy-2-methyl-1-(4-morpholin-4-ylbutyl)-1H-indol-3-yl]methylene}-1-benzofuran-3(2H)-one;
4-hydroxy-2-[(1-{4-[4-(2-hydroxyethyl)piperazin-1-yl]butyl}-5-methoxy-2-methyl-1H-indol-3-yl)methylene]-1-benzofuran-3(2H)-one;
2-[(6-bromo-1H-indol-3-yl)methylene]-4,6-dihydroxy-1-benzofuran-3(2H)-one;
6,7-dihydroxy-2-({5-methoxy-2-methyl-1-[4-(4-methylpiperazin-1-yl)butyl]-1H-indol-3-yl}methylene)-1-benzofuran-3(2H)-one;
6,7-dihydroxy-2-{[5-methoxy-2-methyl-1-(4-morpholin-4-ylbutyl)-1H-indol-3-yl]methylene}-1-benzofuran-3(2H)-one;
6,7-dihydroxy-2-[(1-{4-[4-(2-hydroxyethyl)piperazin-1-yl]butyl}-5-methoxy-2-methyl-1H-indol-3-yl)methylene]-1-benzofuran-3(2H)-one;
4,6-dihydroxy-2-{[1-(2-morpholin-4-ylethyl)-2-phenyl-1H-indol-3-yl]methylene}-1-benzofuran-3(2H)-one;
2-({1-[2-(dimethylamino)ethyl]-2-phenyl-1H-indol-3-yl}methylene)-4,6-dihydroxy-1-benzofuran-3(2H)-one;
2-[(1-benzyl-2-phenyl-1H-indol-3-yl)methylene]-4,6-dihydroxy-1-benzofuran-3(2H)-one;
4,6-dihydroxy-2-[(1-isobutyl-2-phenyl-1H-indol-3-yl)methylene]-1-benzofuran-3(2H)-one;
4,6-dihydroxy-2-{[1-(2-methoxyethyl)-2-phenyl-1H-indol-3-yl]methylene}-1-benzofuran-3(2H)-one;
2-{[1-(cyclopropylmethyl)-2-phenyl-1H-indol-3-yl]methylene}-4,6-dihydroxy-1-benzofuran-3(2H)-one;
4,6-dihydroxy-2-{[2-phenyl-1-(pyridin-3-ylmethyl)-1H-indol-3-yl]methylene}-1-benzofuran-3(2H)-one;
4,6-dihydroxy-2-{[2-phenyl-1-(pyridin-4-ylmethyl)-1H-indol-3-yl]methylene}-1-benzofuran-3(2H)-one;
4-{3-[(4,6-dihydroxy-3-oxo-1-benzofuran-2(3H)-ylidene)methyl]-2-phenyl-1H-indol-1-yl}butanenitrile;
2-({1-[3-(dimethylamino)propyl]-2-phenyl-1H-indol-3-yl}methylene)-4,6-dihydroxy-1-benzofuran-3(2H)-one;
4,6-dihydroxy-2-{[2-phenyl-1-(2-pyrrolidin-1-ylethyl)-1H-indol-3-yl]methylene}-1-benzofuran-3(2H)-one;
4,6-dihydroxy-2-{[2-phenyl-1-(2-piperidin-4-ylethyl)-1H-indol-3-yl]methylene}-1-benzofuran-3(2H)-one;
4,6-dihydroxy-2-({1-[2-(4-methylpiperazin-1-yl)ethyl]-2-phenyl-1H-indol-3-yl}methylene)-1-benzofuran-3(2H)-one;
4,6-dihydroxy-2-{[2-phenyl-1-(2-piperazin-1-ylethyl)-1H-indol-3-yl]methylene}-1-benzofuran-3(2H)-one;
2-{3-[(4,6-dihydroxy-3-oxo-1-benzofuran-2(3H)-ylidene)methyl]-2-phenyl-1H-indol-1-yl}acetamide;
4,6-dihydroxy-2-[(2-methyl-5-nitro-1H-indol-3-yl)methylene]-1-benzofuran-3(2H)-one;
4,6-dihydroxy-2-[(5-hydroxy-1H-indol-3-yl)methylene]-1-benzofuran-3(2H)-one;
3-[(4,6-dihydroxy-3-oxo-1-benzofuran-2(3H)-ylidene)methyl]-1H-indole-5-carboxylic acid;
methyl 3-[(4,6-dihydroxy-3-oxo-1-benzofuran-2(3H)-ylidene)methyl]-1H-indole-5-carboxylate;
3-[(4,6-dihydroxy-3-oxo-1-benzofuran-2(3H)-ylidene)methyl]-1H-indole-6-carbonitrile;
2-(5H-[1,3]dioxolo[4,5-f]indol-7-ylmethylene)-4,6-dihydroxy-1-benzofuran-3(2H)-one;
4,6-dihydroxy-2-{[6-(methylsulfonyl)-1H-indol-3-yl]methylene}-1-benzofuran-3(2H)-one;
4,6-dihydroxy-2-[(5-methyl-1H-indol-3-yl)methylene]-1-benzofuran-3(2H)-one;
2-[(4-chloro-1H-indol-3-yl)methylene]-4,6-dihydroxy-1-benzofuran-3(2H)-one;
2-[(6-chloro-1H-indol-3-yl)methylene]-4,6-dihydroxy-1-benzofuran-3(2H)-one;
2-[(7-chloro-1H-indol-3-yl)methylene]-4,6-dihydroxy-1-benzofuran-3(2H)-one;
2-[(4-bromo-1H-indol-3-yl)methylene]-4,6-dihydroxy-1-benzofuran-3(2H)-one;
2-[(5-fluoro-1H-indol-3-yl)methylene]-4,6-dihydroxy-1-benzofuran-3(2H)-one;
2-[(6-fluoro-1H-indol-3-yl)methylene]-4,6-dihydroxy-1-benzofuran-3(2H)-one;
4,6-dihydroxy-2-[(5-iodo-1H-indol-3-yl)methylene]-1-benzofuran-3(2H)-one;
4,6-dihydroxy-2-[(5-nitro-1H-indol-3-yl)methylene]-1-benzofuran-3(2H)-one;
4,6-dihydroxy-2-[(6-nitro-1H-indol-3-yl)methylene]-1-benzofuran-3(2H)-one;
4,6-dihydroxy-2-[(7-nitro-1H-indol-3-yl)methylene]-1-benzofuran-3(2H)-one;
2-[(5,6-dimethoxy-1H-indol-3-yl)methylene]-4,6-dihydroxy-1-benzofuran-3(2H)-one;
3-[(4,6-dihydroxy-3-oxo-1-benzofuran-2(3H)-ylidene)methyl]-1H-indole-5-carbonitrile;
N-{3-[(4,6-dihydroxy-3-oxo-1-benzofuran-2(3H)-ylidene)methyl]-1H-indol-5-yl}-2-furamide;
4,6-dihydroxy-2-[(5-methoxy-2,6-dimethyl-1H-indol-3-yl)methylene]-1-benzofuran-3(2H)-one;
4,6-dihydroxy-2-[(5-methoxy-1,2,6-trimethyl-1H-indol-3-yl)methylene]-1-benzofuran-3(2H)-one;
4,6-dihydroxy-2-[(6-methoxy-1H-indol-3-yl)methylene]-1-benzofuran-3(2H)-one;
2-[(7-ethyl-1H-indol-3-yl)methylene]-4,6-dihydroxy-1-benzofuran-3(2H)-one;
3-[(4,6-dihydroxy-3-oxo-1-benzofuran-2(3H)-ylidene)methyl]-1-methyl-1H-indole-4-carbonitrile;
4,6-dihydroxy-2-[(1-methyl-2-pyridin-3-yl-1H-indol-3-yl)methylene]-1-benzofuran-3(2H)-one;
4,6-dihydroxy-2-[(1-methyl-2-pyridin-4-yl-1H-indol-3-yl)methylene]-1-benzofuran-3(2H)-one;
2-{[2-(3,5-dimethylisoxazol-4-yl)-1-methyl-1H-indol-3-yl]methylene}-4,6-dihydroxy-1-benzofuran-3(2H)-one;
4,6-dihydroxy-2-{[2-(3-hydroxyphenyl)-1-methyl-1H-indol-3-yl]methylene}-1-benzofuran-3(2H)-one;
4,6-dihydroxy-2-{[2-(4-hydroxyphenyl)-1-methyl-1H-indol-3-yl]methylene}-1-benzofuran-3(2H)-one;
4,6-dihydroxy-2-{[1-methyl-2-(3-thienyl)-1H-indol-3-yl]methylene}-1-benzofuran-3(2H)-one;
4,6-dihydroxy-2-{[2-(4-methoxyphenyl)-1-methyl-1H-indol-3-yl]methylene}-1-benzofuran-3(2H)-one;
4,6-dihydroxy-2-{[2-(3-methoxyphenyl)-1-methyl-1H-indol-3-yl]methylene}-1-benzofuran-3(2H)-one;
2-{[2-(3-fluorophenyl)-1-methyl-1H-indol-3-yl]methylene}-4,6-dihydroxy-1-benzofuran-3(2H)-one;
2-({2-[4-(dimethylamino)phenyl]-1-methyl-1H-indol-3-yl}methylene)-4,6-dihydroxy-1-benzofuran-3(2H)-one;
2-{[2-(3-chloro-4-fluorophenyl)-1-methyl-1H-indol-3-yl]methylene}-4,6-dihydroxy-1-benzofuran-3(2H)-one;
2-{[2-(4-fluorophenyl)-1-methyl-1H-indol-3-yl]methylene}-4,6-dihydroxy-1-benzofuran-3(2H)-one;
2-{[2-(4-chlorophenyl)-1-methyl-1H-indol-3-yl]methylene}-4,6-dihydroxy-1-benzofuran-3(2H)-one;
4,6-dihydroxy-2-[(2-pyridin-3-yl-1H-indol-3-yl)methylene]-1-benzofuran-3(2H)-one;
4,6-dihydroxy-2-[(2-pyridin-4-yl-1H-indol-3-yl)methylene]-1-benzofuran-3(2H)-one;
2-{[2-(3,5-dimethylisoxazol-4-yl)-1H-indol-3-yl]methylene}-4,6-dihydroxy-1-benzofuran-3(2H)-one;
4,6-dihydroxy-2-{[2-(3-hydroxyphenyl)-1H-indol-3-yl]methylene}-1-benzofuran-3(2H)-one;
4,6-dihydroxy-2-{[2-(4-hydroxyphenyl)-1H-indol-3-yl]methylene}-1-benzofuran-3(2H)-one;
4,6-dihydroxy-2-{[2-(3-thienyl)-1H-indol-3-yl]methylene}-1-benzofuran-3(2H)-one;
4,6-dihydroxy-2-{[2-(4-methoxyphenyl)-1H-indol-3-yl]methylene}-1-benzofuran-3(2H)-one;
4,6-dihydroxy-2-{[2-(3-methoxyphenyl)-1H-indol-3-yl]methylene}-1-benzofuran-3(2H)-one;
2-{[2-(3-fluorophenyl)-1H-indol-3-yl]methylene}-4,6-dihydroxy-1-benzofuran-3(2H)-one;
2-({2-[4-(dimethylamino)phenyl]-1H-indol-3-yl}methylene)-4,6-dihydroxy-1-benzofuran-3(2H)-one;
2-{[2-(3-chloro-4-fluorophenyl)-1H-indol-3-yl]methylene}-4,6-dihydroxy-1-benzofuran-3(2H)-one;
2-[(1-ethyl-2-phenyl-1H-indol-3-yl)methylene]-4,6-dihydroxy-1-benzofuran-3(2H)-one;
4,6-dihydroxy-2-[(2-phenyl-1-propyl-1H-indol-3-yl)methylene]-1-benzofuran-3(2H)-one;
2-[(5-chloro-2-methyl-1H-indol-3-yl)methylene]-4,6-dihydroxy-1-benzofuran-3(2H)-one;
2-[(7-bromo-2-methyl-1H-indol-3-yl)methylene]-4,6-dihydroxy-1-benzofuran-3(2H)-one;
2-[(5-fluoro-2-methyl-1H-indol-3-yl)methylene]-4,6-dihydroxy-1-benzofuran-3(2H)-one;
4,6-dihydroxy-2-[(5-methoxy-4-methyl-1H-indol-3-yl)methylene]-1-benzofuran-3(2H)-one;
3-[(4,6-dihydroxy-3-oxo-1-benzofuran-2(3H)-ylidene)methyl]-1H-indole-4-carbonitrile;
4,6-dihydroxy-2-{[6-(trifluoromethyl)-1H-indol-3-yl]methylene}-1-benzofuran-3(2H)-one;
methyl 3-[(4,6-dihydroxy-3-oxo-1-benzofuran-2(3H)-ylidene)methyl]-1H-indole-4-carboxylate;
4,6-dihydroxy-2-[(1-methyl-2-pyridin-2-yl-1H-indol-3-yl)methylene]-1-benzofuran-3(2H)-one;
5-{3-[(4,6-dihydroxy-3-oxo-1-benzofuran-2(3H)-ylidene)methyl]-2-phenyl-1H-indol-1-yl}pentanenitrile;
6-{3-[(4,6-dihydroxy-3-oxo-1-benzofuran-2(3H)-ylidene)methyl]-2-phenyl-1H-indol-1-yl}hexanenitrile;
4-{3-[(4,6-dihydroxy-3-oxo-1-benzofuran-2(3H)-ylidene)methyl]-2-pyridin-3-yl-1H-indol-1-yl}butanenitrile;
2-[(5-fluoro-1-methyl-1H-indol-3-yl)methylene]-4,6-dihydroxy-1-benzofuran-3(2H)-one;
4,6-dihydroxy-2-[(1-methyl-5-nitro-1H-indol-3-yl)methylene]-1-benzofuran-3(2H)-one;
4,6-dihydroxy-2-[(1-methyl-7-nitro-1H-indol-3-yl)methylene]-1-benzofuran-3(2H)-one;
4-{4-bromo-3-[(4,6-dihydroxy-3-oxo-1-benzofuran-2(3H)-ylidene)methyl]-1H-indol-1-yl}butanenitrile;
4-{3-[(4,6-dihydroxy-3-oxo-1-benzofuran-2(3H)-ylidene)methyl]-5-fluoro-1H-indol-1-yl}butanenitrile;
4-{3-[(4,6-dihydroxy-3-oxo-1-benzofuran-2(3H)-ylidene)methyl]-7-ethyl-1H-indol-1-yl}butanenitrile;
2-[(5-chloro-1,2-dimethyl-1H-indol-3-yl)methylene]-4,6-dihydroxy-1-benzofuran-3(2H)-one;
2-[(7-bromo-1,2-dimethyl-1H-indol-3-yl)methylene]-4,6-dihydroxy-1-benzofuran-3(2H)-one;
2-[(5-fluoro-1,2-dimethyl-1H-indol-3-yl)methylene]-4,6-dihydroxy-1-benzofuran-3(2H)-one;
4,6-dihydroxy-2-[(5-methoxy-1,4-dimethyl-1H-indol-3-yl)methylene]-1-benzofuran-3(2H)-one;
4,6-dihydroxy-2-{[1-methyl-6-(trifluoromethyl)-1H-indol-3-yl]methylene}-1-benzofuran-3(2H)-one;
4-{5-chloro-3-[(4,6-dihydroxy-3-oxo-1-benzofuran-2(3H)-ylidene)methyl]-2-methyl-1H-indol-1-yl}butanenitrile;
4-{3-[(4,6-dihydroxy-3-oxo-1-benzofuran-2(3H)-ylidene)methyl]-5-methoxy-4-methyl-1H-indol-1-yl}butanenitrile;
4-{3-[(4,6-dihydroxy-3-oxo-1-benzofuran-2(3H)-ylidene)methyl]-2-methyl-1H-indol-1-yl}butanenitrile;
4-{3-[(4,6-dihydroxy-3-oxo-1-benzofuran-2(3H)-ylidene)methyl]-1H-indol-1-yl}butanenitrile;
2-{[7-(benzyloxy)-1H-indol-3-yl]methylene}-4,6-dihydroxy-1-benzofuran-3(2H)-one;
2-{[4-(benzyloxy)-1H-indol-3-yl]methylene}-4,6-dihydroxy-1-benzofuran-3(2H)-one;
2-[(7-bromo-1H-indol-3-yl)methylene]-4,6-dihydroxy-1-benzofuran-3(2H)-one;
methyl 3-[(4,6-dihydroxy-3-oxo-1-benzofuran-2(3H)-ylidene)methyl]-1H-indole-7-carboxylate;
4,6-dihydroxy-2-[(7-hydroxy-1H-indol-3-yl)methylene]-1-benzofuran-3(2H)-one;
2-[(5-bromo-1-methyl-1H-indol-3-yl)methylene]-4,6-dihydroxy-1-benzofuran-3(2H)-one;
2-[(7-bromo-1-methyl-1H-indol-3-yl)methylene]-4,6-dihydroxy-1-benzofuran-3(2H)-one;
methyl 3-[(4,6-dihydroxy-3-oxo-1-benzofuran-2(3H)-ylidene)methyl]-1-methyl-1H-indole-7-carboxylate;
4,6-dihydroxy-2-[(7-methoxy-1-methyl-1H-indol-3-yl)methylene]-1-benzofuran-3(2H)-one;
2-[(4-chloro-1-methyl-1H-indol-3-yl)methylene]-4,6-dihydroxy-1-benzofuran-3(2H)-one;
2-[(4-bromo-1-methyl-1H-indol-3-yl)methylene]-4,6-dihydroxy-1-benzofuran-3(2H)-one;
4,6-dihydroxy-2-[(4-hydroxy-1-methyl-1H-indol-3-yl)methylene]-1-benzofuran-3(2H)-one;
4,6-dihydroxy-2-[(4-methoxy-1-methyl-1H-indol-3-yl)methylene]-1-benzofuran-3(2H)-one;
2-{[4-(benzyloxy)-1-methyl-1H-indol-3-yl]methylene}-4,6-dihydroxy-1-benzofuran-3(2H)-one;
4-{3-[(4,6-dihydroxy-3-oxo-1-benzofuran-2(3H)-ylidene)methyl]-4-hydroxy-1H-indol-1-yl}butanenitrile;
4,6-dihydroxy-2-[(2-{4-[2-(2-methoxyethoxy)ethoxy]phenyl}-1-methyl-1H-indol-3-;
2-({2-[4-(benzyloxy)phenyl]-1-methyl-1H-indol-3-yl}methylene)-4,6-dihydroxy-1-benzofuran-3(2H)-one;
4,6-dihydroxy-2-{[2-(4-isopropoxyphenyl)-1-methyl-1H-indol-3-yl]methylene}-1-benzofuran-3(2H)-one;
3-[(4,6-dihydroxy-3-oxo-1-benzofuran-2(3H)-ylidene)methyl]-1-(2-morpholin-4-ylethyl)-1H-indole-4-carbonitrile;
3-[(4,6-dihydroxy-3-oxo-1-benzofuran-2(3H)-ylidene)methyl]-1-(pyridin-4-ylmethyl)-1H-indole-4-carbonitrile;
1-(3-cyanopropyl)-3-[(4,6-dihydroxy-3-oxo-1-benzofuran-2(3H)-ylidene)methyl]-1H-indole-4-carbonitrile;
3-[(4,6-dihydroxy-3-oxo-1-benzofuran-2(3H)-ylidene)methyl]-1-[2-(dimethylamino)ethyl]-1H-indole-4-carbonitrile;
3-[(4,6-dihydroxy-3-oxo-1-benzofuran-2(3H)-ylidene)methyl]-1-(2-methoxyethyl)-1H-indole-4-carbonitrile;
methyl 3-[(4,6-dihydroxy-3-oxo-1-benzofuran-2(3H)-ylidene)methyl]-1-methyl-1H-indole-4-carboxylate;
4,6-dihydroxy-2-[(1-methyl-4-phenyl-1H-indol-3-yl)methylene]-1-benzofuran-3(2H)-one;
4,6-dihydroxy-2-[(4-phenyl-1H-indol-3-yl)methylene]-1-benzofuran-3(2H)-one;
(2Z)-6,7-dihydroxy-2-[(2-phenyl-1H-indol-3-yl)methylene]-1-benzofuran-3(2H)-one;
(2Z)-2-(1H-indol-3-ylmethylene)-1-benzothiophen-3(2H)-one;
(2Z)-2-[(2-methyl-1H-indol-3-yl)methylene]-1-benzothiophen-3(2H)-one;
(2Z)-2-[(2-phenyl-1H-indol-3-yl)methylene]-1-benzothiophen-3(2H)-one;
(2Z)-2-[(1-methyl-2-phenyl-1H-indol-3-yl)methylene]-1-benzothiophen-3(2H)-one;
(2Z)-2-{[2-(2-naphthyl)-1H-indol-3-yl]methylene}-1-benzothiophen-3(2H)-one;
(2Z)-2-{[2-(4-fluorophenyl)-1H-indol-3-yl]methylene}-1-benzothiophen-3(2H)-one;
(2Z)-2-[(6-methyl-1H-indol-3-yl)methylene]-1-benzothiophen-3(2H)-one;
(2Z)-2-[(7-methyl-1H-indol-3-yl)methylene]-1-benzothiophen-3(2H)-one;
(2Z)-5-chloro-2-(1H-indol-3-ylmethylene)-1-benzothiophen-3(2H)-one;
(2Z)-5-chloro-2-{[2-(4-chlorophenyl)-1H-indol-3-yl]methylene}-1-benzothiophen-3(2H)-one;
(2Z)-5-chloro-2-{[2-(2-naphthyl)-1H-indol-3-yl]methylene}-1-benzothiophen-3(2H)-one;
(2Z)-5-chloro-2-{[2-(4-fluorophenyl)-1H-indol-3-yl]methylene}-1-benzothiophen-3(2H)-one;
(2Z)-2-[(1-benzyl-1H-indol-3-yl)methylene]-5-chloro-1-benzothiophen-3(2H)-one;
(2Z)-2-{[5-(benzyloxy)-1H-indol-3-yl]methylene}-5-chloro-1-benzothiophen-3(2H)-one;
(2Z)-2-(1H-indol-3-ylmethylene)-5-methyl-1-benzothiophen-3(2H)-one;
(2Z)-5-methyl-2-[(2-phenyl-1H-indol-3-yl)methylene]-1-benzothiophen-3(2H)-one;
(2Z)-2-{[2-(4-chlorophenyl)-1H-indol-3-yl]methylene}-5-methyl-1-benzothiophen-3(2H)-one;
(2Z)-5-methyl-2-[(6-methyl-1H-indol-3-yl)methylene]-1-benzothiophen-3(2H)-one;
(2Z)-5-methyl-2-[(7-methyl-1H-indol-3-yl)methylene]-1-benzothiophen-3(2H)-one;
(2Z)-2-[(5-bromo-1H-indol-3-yl)methylene]-5-methyl-1-benzothiophen-3(2H)-one;
(2Z)-2-{[5-(benzyloxy)-1H-indol-3-yl]methylene}-5-methyl-1-benzothiophen-3(2H)-one;
5-methyl-2-{[2-(2-naphthyl)-1H-indol-3-yl]methylene}-1-benzothiophen-3(2H)-one;
2-{[2-(4-fluorophenyl)-1H-indol-3-yl]methylene}-5-methyl-1-benzothiophen-3(2H)-one;
5-methyl-2-[(2-methyl-5-nitro-1H-indol-3-yl)methylene]-1-benzothiophen-3(2H)-one;
5-methyl-2-[(1-methyl-1H-indol-3-yl)methylene]-1-benzothiophen-3(2H)-one;
(2Z)-2-({4-[4′-(aminomethyl)biphenyl-4-yl]-1-methyl-1H-indol-3-yl}methylene)-4,6-dihydroxy-1-benzofuran-3(2H)-one;
(2Z)-2-[(4-{4′-[(dimethylamino)methyl]biphenyl-4-yl}-1-methyl-1H-indol-3-yl)methylene]-4,6-dihydroxy-1-benzofuran-3(2H)-one;
(2Z)-6-hydroxy-2-({5-methoxy-2-methyl-1-[2-(4-methylpiperazin-1-yl)ethyl]-1H-indol-3-yl}methylene)-1-benzofuran-3(2H)-one;
(2Z)-2-({2-cyclopropyl-5-methoxy-1-[2-(4-methylpiperazin-1-yl)ethyl]-1H-indol-3-yl}methylene)-4,6-dihydroxy-1-benzofuran-3(2H)-one;
(2Z)-4-hydroxy-2-[(5-methoxy-2-phenyl-1H-indol-3-yl)methylene]-1-benzofuran-3(2H)-one;
(2Z)-2-({1-[3-(dimethylamino)propyl]-5-methoxy-2-methyl-1H-indol-3-yl}methylene)-4,6-dihydroxy-1-benzofuran-3(2H)-one;
3-[(Z)-(4,6-dihydroxy-3-oxo-1-benzofuran-2(3H)-ylidene)methyl]-1-[2-(3-hydroxypyrrolidin-1-yl)ethyl]-5-methoxy-N,N-dimethyl-1H-indole-2-carboxamide;
(2Z)-2-({1-[3-(dimethylamino)propyl]-5-methoxy-2-methyl-1H-indol-3-yl}methylene)-6-hydroxy-1-benzofuran-3(2H)-one;
(2Z)-4,6-dihydroxy-2-({5-methoxy-1-[2-(4-methylpiperazin-1-yl)ethyl]-1H-indol-3-yl}methylene)-1-benzofuran-3(2H)-one;
(2Z)-4,6-dihydroxy-2-({5-methoxy-2-methyl-1-[2-(4-methylpiperazin-1-yl)ethyl]-1H-indol-3-yl}methylene)-1-benzofuran-3(2H)-one;
(2Z)-6-hydroxy-2-[(5-methoxy-2-phenyl-1H-indol-3-yl)methylene]-1-benzofuran-3(2H)-one;
3-[(Z)-(4,6-dihydroxy-3-oxo-1-benzofuran-2(3H)-ylidene)methyl]-1-[2-(4-hydroxypiperidin-1-yl)ethyl]-5-methoxy-N,N-dimethyl-1H-indole-2-carboxamide;
(2Z)-4,6-dihydroxy-2-({1-[2-(1H-imidazol-1-yl)ethyl]-5-methoxy-1H-indol-3-yl}methylene)-1-benzofuran-3(2H)-one;
(2Z)-2-{[2-cyclopropyl-1-(2-hydroxyethyl)-5-methoxy-1H-indol-3-yl]methylene}-4,6-dihydroxy-1-benzofuran-3(2H)-one;
3-[(Z)-(4,6-dihydroxy-3-oxo-1-benzofuran-2(3H)-ylidene)methyl]-1-[2-(1H-imidazol-1-yl)ethyl]-5-methoxy-N,N-dimethyl-1H-indole-2-carboxamide;
(2Z)-2-({2-cyclopropyl-1-[2-(4-hydroxypiperidin-1-yl)ethyl]-5-methoxy-1H-indol-3-yl}methylene)-4,6-dihydroxy-1-benzofuran-3(2H)-one;
3-[(Z)-(4,6-dihydroxy-3-oxo-1-benzofuran-2(3H)-ylidene)methyl]-5-methoxy-N,N-dimethyl-1-[2-(4-methylpiperazin-1-yl)ethyl]-1H-indole-2-carboxamide;
(2Z)-4,6-dihydroxy-2-{[1-(2-hydroxyethyl)-5-methoxy-1H-indol-3-yl]methylene}-1-benzofuran-3(2H)-one;
2-{3-[(Z)-(4,6-dihydroxy-3-oxo-1-benzofuran-2(3H)-ylidene)methyl]-5-methoxy-1H-indol-1-yl}-N,N-dimethylacetamide;
(2Z)-4,6-dihydroxy-2-({1-[2-(4-hydroxypiperidin-1-yl)ethyl]-5-methoxy-1H-indol-3-yl}methylene)-1-benzofuran-3(2H)-one;
(2Z)-4,6-dihydroxy-2-({1-[2-(4-hydroxypiperidin-1-yl)ethyl]-5-methoxy-2-methyl-1H-indol-3-yl}methylene)-1-benzofuran-3(2H)-one;
(2Z)-4,6-dihydroxy-2-{[1-(2-hydroxyethyl)-5-methoxy-2-methyl-1H-indol-3-yl]methylene}-1-benzofuran-3(2H)-one;
2-{3-[(Z)-(4,6-dihydroxy-3-oxo-1-benzofuran-2(3H)-ylidene)methyl]-5-methoxy-2-methyl-1H-indol-1-yl}-N,N-dimethylacetamide;
(2Z)-2-({1-[3-(dimethylamino)propyl]-5-methoxy-1H-indol-3-yl}methylene)-4,6-dihydroxy-1-benzofuran-3(2H)-one;
(2Z)-2-({1-[3-(dimethylamino)propyl]-5-methoxy-1H-indol-3-yl}methylene)-6-hydroxy-1-benzofuran-3(2H)-one;
3-[(Z)-(4,6-dihydroxy-3-oxo-1-benzofuran-2(3H)-ylidene)methyl]-1-[2-(dimethylamino)-2-oxoethyl]-5-methoxy-N,N-dimethyl-1H-indole-2-carboxamide;
(2Z)-4,6-dihydroxy-2-({1-[2-(3-hydroxypyrrolidin-1-yl)ethyl]-5-methoxy-1H-indol-3-yl}methylene)-1-benzofuran-3(2H)-one;
(2Z)-4,6-dihydroxy-2-({1-[2-(3-hydroxypyrrolidin-1-yl)ethyl]-5-methoxy-2-methyl-1H-indol-3-yl}methylene)-1-benzofuran-3(2H)-one;
(2Z)-4,6-dihydroxy-2-({1-[2-(1H-imidazol-1-yl)ethyl]-5-methoxy-2-methyl-1H-indol-3-yl}methylene)-1-benzofuran-3(2H)-one;
2-{3-[(Z)-(4,6-dihydroxy-3-oxo-1-benzofuran-2(3H)-ylidene)methyl]-5-methoxy-2-(1-methyl-1H-pyrazol-4-yl)-1H-indol-1-yl}-N,N-dimethylacetamide;
(2Z)-4,6-dihydroxy-2-{[5-methoxy-2-(1-methyl-1H-pyrazol-4-yl)-1H-indol-3-yl]methylene}-1-benzofuran-3(2H)-one;
(2Z)-4,6-dihydroxy-2-({5-methoxy-2-[(4-methylpiperazin-1-yl)carbonyl]-1H-indol-3-yl}methylene)-1-benzofuran-3(2H)-one;
(2Z)-4,6-dihydroxy-2-{[1-(2-hydroxyethyl)-5-methoxy-2-(trifluoromethyl)-1H-indol-3-yl]methylene}-1-benzofuran-3(2H)-one;
2-{3-[(Z)-(4,6-dihydroxy-3-oxo-1-benzofuran-2(3H)-ylidene)methyl]-5-methoxy-2-(trifluoromethyl)-1H-indol-1-yl}-N,N-dimethylacetamide;
(2Z)-2-({2-cyclopropyl-1-[2-(1H-imidazol-1-yl)ethyl]-5-methoxy-1H-indol-3-yl}methylene)-4,6-dihydroxy-1-benzofuran-3(2H)-one;
3-[(Z)-(6-hydroxy-3-oxo-1-benzofuran-2(3H)-ylidene)methyl]-5-methoxy-N,N-dimethyl-1-[2-(4-methylpiperazin-1-yl)ethyl]-1H-indole-2-carboxamide;
(2Z)-4,6-dihydroxy-2-({5-methoxy-1-[2-(4-methylpiperazin-1-yl)ethyl]-2-(trifluoromethyl)-1H-indol-3-yl}methylene)-1-benzofuran-3(2H)-one;
Z)-4,6-dihydroxy-2-({5-methoxy-1-[2-(1H-pyrazol-1-yl)ethyl]-2-(trifluoromethyl)-1H-indol-3-yl}methylene)-1-benzofuran-3(2H)-one;
(2Z)-6-hydroxy-2-({5-methoxy-2-methyl-1-[2-(4-methylpiperazin-1-yl)ethyl]-1H-indol-3-yl}methylene)-4-methyl-1-benzofuran-3(2H)-one;
3-[(Z)-(4,6-dihydroxy-3-oxo-1-benzofuran-2(3H)-ylidene)methyl]-5-methoxy-1H-indole-2-carboxylic acid;
(2Z)-2-({2-cyclopropyl-5-methoxy-1-[2-(1H-pyrazol-1-yl)ethyl]-1H-indol-3-yl}methylene)-4,6-dihydroxy-1-benzofuran-3(2H)-one;
(2Z)-4,6-dihydroxy-2-({5-methoxy-1-[2-(4-methylpiperazin-1-yl)ethyl]-2-(1-methyl-1H-pyrazol-4-yl)-1H-indol-3-yl}methylene)-1-benzofuran-3(2H)-one;
(2Z)-2-({2-cyclopropyl-1-[2-(3-hydroxypyrrolidin-1-yl)ethyl]-5-methoxy-1H-indol-3-yl}methylene)-4,6-dihydroxy-1-benzofuran-3(2H)-one;
3-[(Z)-(4,6-dihydroxy-3-oxo-1-benzofuran-2(3H)-ylidene)methyl]-1-[3-(dimethylamino)propyl]-5-methoxy-N,N-dimethyl-1H-indole-2-carboxamide;
(2Z)-2-({2-(3,5-dimethylisoxazol-4-yl)-5-methoxy-1-[2-(4-methylpiperazin-1-yl)ethyl]-1H-indol-3-yl}methylene)-4,6-dihydroxy-1-benzofuran-3(2H)-one;
1-[3-(dimethylamino)propyl]-3-[(Z)-(6-hydroxy-3-oxo-1-benzofuran-2(3H)-ylidene)methyl]-5-methoxy-N,N-dimethyl-1H-indole-2-carboxamide;
(2Z)-4,6-dihydroxy-2-({5-methoxy-2-[(4-methylpiperazin-1-yl)methyl]-1H-indol-3-yl}methylene)-1-benzofuran-3(2H)-one;
(2Z)-6-hydroxy-2-({5-methoxy-2-methyl-1-[2-(4-methylpiperazin-1-yl)ethyl]-1H-indol-3-yl}methylene)-7-methyl-1-benzofuran-3(2H)-one;
3-[(Z)-(4,6-dihydroxy-3-oxo-1-benzofuran-2(3H)-ylidene)methyl]-1-(2-hydroxyethyl)-5-methoxy-N,N-dimethyl-1H-indole-2-carboxamide;
(2Z)-5-bromo-6-hydroxy-2-({5-methoxy-2-methyl-1-[2-(4-methylpiperazin-1-yl)ethyl]-1H-indol-3-yl}methylene)-1-benzofuran-3(2H)-one;
(2Z)-5-bromo-2-({1-[3-(dimethylamino)propyl]-5-methoxy-2-methyl-1H-indol-3-yl}methylene)-6-hydroxy-1-benzofuran-3(2H)-one;
(2Z)-5-fluoro-6-hydroxy-2-({5-methoxy-2-methyl-1-[2-(4-methylpiperazin-1-yl)ethyl]-1H-indol-3-yl}methylene)-1-benzofuran-3(2H)-one;
(2Z)-2-({1-[3-(dimethylamino)propyl]-5-methoxy-2-methyl-1H-indol-3-yl}methylene)-5-fluoro-6-hydroxy-1-benzofuran-3(2H)-one;
(2Z)-2-({2-[(dimethylamino)methyl]-5-methoxy-1H-indol-3-yl}methylene)-4,6-dihydroxy-1-benzofuran-3(2H)-one;
(2Z)-4,6-dihydroxy-2-[(5-methoxy-2-pyrimidin-5-yl-1H-indol-3-yl)methylene]-1-benzofuran-3(2H)-one;
(2Z)-2-({2-cyclopropyl-5-methoxy-1-[2-(1H-pyrazol-1-yl)ethyl]-1H-indol-3-yl}methylene)-6-hydroxy-1-benzofuran-3(2H)-one;
(2Z)-6-hydroxy-2-{[5-methoxy-2-methyl-1-(3-pyrrolidin-1-ylpropyl)-1H-indol-3-yl]methylene}-1-benzofuran-3(2H)-one;
(2Z)-6-hydroxy-2-{[5-methoxy-2-methyl-1-(3-piperidin-1-ylpropyl)-1H-indol-3-yl]methylene}-1-benzofuran-3(2H)-one;
(2Z)-2-{[1-(3-azepan-1-ylpropyl)-5-methoxy-2-methyl-1H-indol-3-yl]methylene}-6-hydroxy-1-benzofuran-3(2H)-one;
(2Z)-4-fluoro-6-hydroxy-2-({5-methoxy-2-methyl-1-[2-(4-methylpiperazin-1-yl)ethyl]-1H-indol-3-yl}methylene)-1-benzofuran-3(2H)-one;
(2Z)-2-{[2-(3,5-dimethylisoxazol-4-yl)-5-methoxy-1H-indol-3-yl]methylene}-4,6-dihydroxy-1-benzofuran-3(2H)-one;
(2Z)-7-chloro-2-({1-[3-(dimethylamino)propyl]-5-methoxy-2-methyl-1H-indol-3-yl}methylene)-6-hydroxy-1-benzofuran-3(2H)-one;
(2Z)-2-({2-cyclopropyl-5-methoxy-1-[2-(4-methylpiperazin-1-yl)ethyl]-1H-indol-3-yl}methylene)-6-hydroxy-1-benzofuran-3(2H)-one;
(2Z)-2-{[4-(4-fluorophenyl)-1H-indol-3-yl]methylene}-4,6-dihydroxy-1-benzofuran-3(2H)-one;
(2Z)-2-{[4-(4-fluorophenyl)-1-methyl-1H-indol-3-yl]methylene}-4,6-dihydroxy-1-benzofuran-3(2H)-one;
(2Z)-2-{[4-(3-fluorophenyl)-1-methyl-1H-indol-3-yl]methylene}-4,6-dihydroxy-1-benzofuran-3(2H)-one;
(2Z)-2-{[4-(2-fluorophenyl)-1-methyl-1H-indol-3-yl]methylene}-4,6-dihydroxy-1-benzofuran-3(2H)-one;
3-{3-[(Z)-(4,6-dihydroxy-3-oxo-1-benzofuran-2(3H)-ylidene)methyl]-1-methyl-1H-indol-4-yl}benzonitrile;
4-{3-[(Z)-(4,6-dihydroxy-3-oxo-1-benzofuran-2(3H)-ylidene)methyl]-1-methyl-1H-indol-4-yl}benzonitrile;
(2Z)-4,6-dihydroxy-2-{[4-(6-methoxypyridin-3-yl)-1-methyl-1H-indol-3-yl]methylene}-1-benzofuran-3(2H)-one;
(2Z)-2-{[4-(4-aminophenyl)-1-methyl-1H-indol-3-yl]methylene}-4,6-dihydroxy-1-benzofuran-3(2H)-one;
(2Z)-4,6-dihydroxy-2-{[4-(4-methoxyphenyl)-1H-indol-3-yl]methylene}-1-benzofuran-3(2H)-one;
(2Z)-2-{[4-(4-acetylphenyl)-1-methyl-1H-indol-3-yl]methylene}-4,6-dihydroxy-1-benzofuran-3(2H)-one;
(2Z)-4,6-dihydroxy-2-[(1-methyl-4-pyridin-4-yl-1H-indol-3-yl)methylene]-1-benzofuran-3(2H)-one;
(2Z)-4,6-dihydroxy-2-{[1-methyl-4-(3-thienyl)-1H-indol-3-yl]methylene}-1-benzofuran-3(2H)-one;
(2Z)-4,6-dihydroxy-2-[(1-methyl-4-pyridin-3-yl-1H-indol-3-yl)methylene]-1-benzofuran-3(2H)-one;
(2Z)-4,6-dihydroxy-2-{[4-(4-methoxyphenyl)-1-methyl-1H-indol-3-yl]methylene}-1-benzofuran-3(2H)-one;
4-{3-[(Z)-(4,6-dihydroxy-3-oxo-1-benzofuran-2(3H)-ylidene)methyl]-1-methyl-1H-indol-4-yl}benzamide;
(2Z)-2-{[4-(3-furyl)-1-methyl-1H-indol-3-yl]methylene}-4,6-dihydroxy-1-benzofuran-3(2H)-one;
(2Z)-4,6-dihydroxy-2-{[4-(4-hydroxyphenyl)-1-methyl-1H-indol-3-yl]methylene}-1-benzofuran-3(2H)-one;
(2Z)-2-{[4-(6-aminopyridin-3-yl)-1-methyl-1H-indol-3-yl]methylene}-4,6-dihydroxy-1-benzofuran-3(2H)-one;
(2Z)-4,6-dihydroxy-2-{[4-(4-isopropoxyphenyl)-1-methyl-1H-indol-3-yl]methylene}-1-benzofuran-3(2H)-one;
ethyl 4-{3-[(Z)-(4,6-dihydroxy-3-oxo-1-benzofuran-2(3H)-ylidene)methyl]-1-methyl-1H-indol-4-yl}benzoate;
N-(4-{3-[(Z)-(4,6-dihydroxy-3-oxo-1-benzofuran-2(3H)-ylidene)methyl]-1-methyl-1H-indol-4-yl}phenyl)acetamide;
(2Z)-2-({4-[4-(dimethylamino)phenyl]-1-methyl-1H-indol-3-yl}methylene)-4,6-dihydroxy-1-benzofuran-3(2H)-one;
(2Z)-4,6-dihydroxy-2-{[1-methyl-4-(6-morpholin-4-ylpyridin-3-yl)-1H-indol-3-yl]methylene}-1-benzofuran-3(2H)-one;
4-{3-[(Z)-(4,6-dihydroxy-3-oxo-1-benzofuran-2(3H)-ylidene)methyl]-1-methyl-1H-indol-4-yl}-N-[3-(dimethylamino)propyl]benzamide;
N-(3-{3-[(Z)-(4,6-dihydroxy-3-oxo-1-benzofuran-2(3H)-ylidene)methyl]-1-methyl-1H-indol-4-yl}phenyl)acetamide;
(2Z)-4,6-dihydroxy-2-({1-methyl-4-[4-(methylamino)phenyl]-1H-indol-3-yl}methylene)-1-benzofuran-3(2H)-one;
(2Z)-4,6-dihydroxy-2-{[4-(3-hydroxyphenyl)-1-methyl-1H-indol-3-yl]methylene}-1-benzofuran-3(2H)-one;
methyl (4-{3-[(Z)-(4,6-dihydroxy-3-oxo-1-benzofuran-2(3H)-ylidene)methyl]-1-methyl-1H-indol-4-yl}phenyl)carbamate;
4-{3-[(Z)-(4,6-dihydroxy-3-oxo-1-benzofuran-2(3H)-ylidene)methyl]-1-methyl-1H-indol-4-yl}-N-methylbenzamide;
1-(4-{3-[(Z)-(4,6-dihydroxy-3-oxo-1-benzofuran-2(3H)-ylidene)methyl]-1-methyl-1H-indol-4-yl}phenyl)-3-methylurea;
3-(4-{3-[(Z)-(4,6-dihydroxy-3-oxo-1-benzofuran-2(3H)-ylidene)methyl]-1-methyl-1H-indol-4-yl}phenyl)-1,1-dimethylurea;
4-{3-[(Z)-(4,6-dihydroxy-3-oxo-1-benzofuran-2(3H)-ylidene)methyl]-1-methyl-1H-indol-4-yl}-N-isopropylbenzamide;
(2Z)-4,6-dihydroxy-2-({1-methyl-4-[4-(pyrrolidin-1-ylcarbonyl)phenyl]-1H-indol-3-yl}methylene)-1-benzofuran-3(2H)-one;
5-{3-[(Z)-(4,6-dihydroxy-3-oxo-1-benzofuran-2(3H)-ylidene)methyl]-1-methyl-1H-indol-4-yl}pyridine-2-carbonitrile;
(2Z)-2-({4-[3-(dimethylamino)phenyl]-1-methyl-1H-indol-3-yl}methylene)-4,6-dihydroxy-1-benzofuran-3(2H)-one;
4-{3-[(Z)-(4,6-dihydroxy-3-oxo-1-benzofuran-2(3H)-ylidene)methyl]-1-methyl-1H-indol-4-yl}-N-(2-furylmethyl)benzamide;
(2Z)-4-hydroxy-2-[(1-methyl-4-phenyl-1H-indol-3-yl)methylene]-1-benzofuran-3(2H)-one;
1-cyclopropyl-3-(4-{3-[(Z)-(4,6-dihydroxy-3-oxo-1-benzofuran-2(3H)-ylidene)methyl]-1-methyl-1H-indol-4-yl}phenyl)urea;
(2Z)-4,6-dihydroxy-2-[(1-methyl-4-{6-[(2-morpholin-4-ylethyl)amino]pyridin-3-yl}-1H-indol-3-yl)methylene]-1-benzofuran-3(2H)-one;
(2Z)-4,6-dihydroxy-2-({1-methyl-4-[1-(2-morpholin-4-ylethyl)-1H-pyrazol-4-yl]-1H-indol-3-yl}methylene)-1-benzofuran-3(2H)-one;
N-(4-{3-[(Z)-(4,6-dihydroxy-3-oxo-1-benzofuran-2(3H)-ylidene)methyl]-1-methyl-1H-indol-4-yl}phenyl)morpholine-4-carboxamide;
methyl [2-({1-[3-(dimethylamino)propyl]-5-methoxy-1H-indol-3-yl}methylene)-3-oxo-2,3-dihydro-1-benzofuran-6-yl]carbamate;
1-[2-({1-[3-(dimethylamino)propyl]-5-methoxy-1H-indol-3-yl}methylene)-3-oxo-2,3-dihydro-1-benzofuran-6-yl]-3-methylurea;
N-[2-({1-[3-(dimethylamino)propyl]-5-methoxy-1H-indol-3-yl}methylene)-3-oxo-2,3-dihydro-1-benzofuran-6-yl]acetamide;
(2Z)-2-[(4-bromo-1-methyl-1H-indol-3-yl)methylene]-4,6-dimethoxy-1-benzofuran-3(2H)-one;
(2Z)-4,6-dimethoxy-2-[(1-methyl-2-phenyl-1H-indol-3-yl)methylene]-1-benzofuran-3(2H)-one;
(2Z)-2-({2-cyclopentyl-5-methoxy-1-[2-(4-methylpiperazin-1-yl)ethyl]-1H-indol-3-yl}methylene)-4,6-dihydroxy-1-benzofuran-3(2H)-one;
Z)-2-({2-cyclopentyl-5-methoxy-1-[2-(4-methylpiperazin-1-yl)ethyl]-1H-indol-3-yl}methylene)-6-hydroxy-1-benzofuran-3(2H)-one;
(2Z)-6-hydroxy-2-({5-methoxy-1-[2-(4-methylpiperazin-1-yl)ethyl]-2-(morpholin-4-ylcarbonyl)-1H-indol-3-yl}methylene)-1-benzofuran-3(2H)-one;
(2Z)-6-bromo-2-({1-[3-(dimethylamino)propyl]-5-methoxy-1H-indol-3-yl}methylene)-1-benzofuran-3(2H)-one;
(2Z)-6-hydroxy-2-[1-(5-methoxy-1H-indol-3-yl)ethylidene]-1-benzofuran-3(2H)-one;
tert-butyl [2-({1-[3-(dimethylamino)propyl]-5-methoxy-1H-indol-3-yl}methylene)-3-oxo-2,3-dihydro-1-benzofuran-6-yl]carbamate;
6-amino-2-({1-[3-(dimethylamino)propyl]-5-methoxy-1H-indol-3-yl}methylene)-1-benzofuran-3(2H)-one;
(2Z)-2-{[4-(2-aminophenyl)-1-methyl-1H-indol-3-yl]methylene}-4,6-dihydroxy-1-benzofuran-3(2H)-one;
(2Z)-4,6-dihydroxy-2-{[1-methyl-4-(4-nitrophenyl)-1H-indol-3-yl]methylene}-1-benzofuran-3(2H)-one;
(2Z)-4-hydroxy-6-methoxy-2-[(1-methyl-2-phenyl-1H-indol-3-yl)methylene]-1-benzofuran-3(2H)-one;
N-[2-({1-[3-(dimethylamino)propyl]-5-methoxy-1H-indol-3-yl}methylene)-3-oxo-2,3-dihydro-1-benzofuran-6-yl]methanesulfonamide;
(2Z)-7-bromo-4-methoxy-2-[(1-methyl-4-phenyl-1H-indol-3-yl)methylene]-1-benzofuran-3(2H)-one;
(2Z)-2-({1-[3-(dimethylamino)propyl]-5-methoxy-1H-indol-3-yl}methylene)-6-(hydroxymethyl)-1-benzofuran-3(2H)-one;
4-{3-[(Z)-(4,6-dihydroxy-3-oxo-1-benzofuran-2(3H)-ylidene)methyl]-1-[2-(4-methylpiperazin-1-yl)ethyl]-1H-indol-4-yl}benzamide;
N-{4-[(2Z)-2-({1-[3-(dimethylamino)propyl]-5-methoxy-1H-indol-3-yl}methylene)-3-oxo-2,3-dihydro-1-benzofuran-6-yl]phenyl}acetamide;
(2Z)-6-(2-aminopyrimidin-5-yl)-2-({1-[3-(dimethylamino)propyl]-5-methoxy-1H-indol-3-yl}methylene)-1-benzofuran-3(2H)-one;
(2Z)-7-bromo-4-hydroxy-2-[(1-methyl-4-phenyl-1H-indol-3-yl)methylene]-1-benzofuran-3(2H)-one;
(2Z)-4-bromo-6-hydroxy-2-({5-methoxy-2-methyl-1-[2-(4-methylpiperazin-1-yl)ethyl]-1H-indol-3-yl}methylene)-1-benzofuran-3(2H)-one;
(2Z)-4,6-dihydroxy-2-({5-methoxy-1-[2-(4-methylpiperazin-1-yl)ethyl]-2-(pyrrolidin-1-ylcarbonyl)-1H-indol-3-yl}methylene)-1-benzofuran-3(2H)-one;
(2Z)-6-hydroxy-4-methoxy-2-[(1-methyl-2-phenyl-1H-indol-3-yl)methylene]-1-benzofuran-3(2H)-one;
6-methoxy-2-[(5-methoxy-1H-indol-3-yl)methylene]-1-benzothiophen-3(2H)-one;
2-[(5-methoxy-1H-indol-3-yl)methylene]-6-(methylthio)-1-benzofuran-3(2H)-one;
2-[(5-methoxy-1H-indol-3-yl)methylene]-6-(methylsulfinyl)-1-benzofuran-3(2H)-one;
2-[(5-methoxy-1H-indol-3-yl)methylene]-6-(methylsulfonyl)-1-benzofuran-3(2H)-one;
3-[(Z)-(4,6-dihydroxy-3-oxo-1-benzofuran-2(3H)-ylidene)methyl]-1-methyl-2-phenyl-1H-indole-4-carbonitrile;
(2Z)-2-({4-[4-(dimethylamino)phenyl]-1-[2-(4-methylpiperazin-1-yl)ethyl]-1H-indol-3-yl}methylene)-4,6-dihydroxy-1-benzofuran-3(2H)-one;
(2Z)-4,6-dihydroxy-2-{[1-methyl-4-(4-methylpiperazin-1-yl)-1H-indol-3-yl]methylene}-1-benzofuran-3(2H)-one;
(2Z)-4-hydroxy-2-[(1-methyl-2-phenyl-1H-indol-3-yl)methylene]-1-benzofuran-3(2H)-one;
(2Z)-6-hydroxy-2-[(1-methyl-4-phenyl-1H-indol-3-yl)methylene]-1-benzofuran-3(2H)-one;
2-{2-cyclopropyl-3-[(Z)-(4,6-dihydroxy-3-oxo-1-benzofuran-2(3H)-ylidene)methyl]-5-methoxy-1H-indol-1-yl}-N,N-dimethylacetamide;
(2Z)-2-({2-cyclobutyl-5-methoxy-1-[2-(4-methylpiperazin-1-yl)ethyl]-1H-indol-3-yl}methylene)-4,6-dihydroxy-1-benzofuran-3(2H)-one;
(2Z)-2-({2-cyclohexyl-5-methoxy-1-[2-(4-methylpiperazin-1-yl)ethyl]-1H-indol-3-yl}methylene)-4,6-dihydroxy-1-benzofuran-3(2H)-one;
(2Z)-5-chloro-2-({1-[3-(dimethylamino)propyl]-5-methoxy-2-methyl-1H-indol-3-yl}methylene)-6-hydroxy-1-benzofuran-3(2H)-one;
(2Z)-2-({1-[3-(dimethylamino)propyl]-5-methoxy-2-methyl-1H-indol-3-yl}methylene)-7-fluoro-6-hydroxy-1-benzofuran-3(2H)-one;
(2Z)-2-({1-[3-(dimethylamino)propyl]-5-methoxy-2-methyl-1H-indol-3-yl}methylene)-6-hydroxy-7-methyl-1-benzofuran-3(2H)-one;
(2Z)-7-chloro-6-hydroxy-2-({5-methoxy-2-methyl-1-[2-(4-methylpiperazin-1-yl)ethyl]-1H-indol-3-yl}methylene)-1-benzofuran-3(2H)-one;
(2Z)-6-hydroxy-2-{[5-methoxy-2-methyl-1-(2-pyrrolidin-1-ylethyl)-1H-indol-3-yl]methylene}-1-benzofuran-3(2H)-one;
(2Z)-6-hydroxy-2-({5-methoxy-2-methyl-1-[2-(2-methylpyrrolidin-1-yl)ethyl]-1H-indol-3-yl}methylene)-1-benzofuran-3(2H)-one;
(2Z)-6-hydroxy-2-{[5-methoxy-2-methyl-1-(2-piperidin-1-ylethyl)-1H-indol-3-yl]methylene}-1-benzofuran-3(2H)-one;
(2Z)-6-hydroxy-2-({5-methoxy-2-methyl-1-[2-(4-methylpiperidin-1-yl)ethyl]-1H-indol-3-yl}methylene)-1-benzofuran-3(2H)-one;
(2Z)-5-chloro-6-hydroxy-2-({5-methoxy-2-methyl-1-[2-(4-methylpiperazin-1-yl)ethyl]-1H-indol-3-yl}methylene)-1-benzofuran-3(2H)-one;
(2Z)-7-fluoro-6-hydroxy-2-({5-methoxy-2-methyl-1-[2-(4-methylpiperazin-1-yl)ethyl]-1H-indol-3-yl}methylene)-1-benzofuran-3(2H)-one;
(2Z)-6-hydroxy-2-({5-methoxy-2-methyl-1-[2-(4-methylpiperazin-1-yl)ethyl]-1H-indol-3-yl}methylene)-5-methyl-1-benzofuran-3(2H)-one;
(2Z)-2-({1-[3-(dimethylamino)propyl]-5-methoxy-2-methyl-1H-indol-3-yl}methylene)-6-hydroxy-5-methyl-1-benzofuran-3(2H)-one;
(2Z)-6-hydroxy-2-({5-methoxy-2-methyl-1-[3-(4-methylpiperidin-1-yl)propyl]-1H-indol-3-yl}methylene)-1-benzofuran-3(2H)-one;
(2Z)-6-hydroxy-2-({5-methoxy-2-methyl-1-[3-(2-methylpyrrolidin-1-yl)propyl]-1H-indol-3-yl}methylene)-1-benzofuran-3(2H)-one;
(2Z)-2-({2-cyclopropyl-1-[3-(dimethylamino)propyl]-5-methoxy-1H-indol-3-yl}methylene)-6-hydroxy-1-benzofuran-3(2H)-one;
(2Z)-4,6-dihydroxy-2-({5-methoxy-1-[2-(4-methylpiperazin-1-yl)ethyl]-2-pyrimidin-5-yl-1H-indol-3-yl}methylene)-1-benzofuran-3(2H)-one;
(2Z)-2-({2-cyclopropyl-5-methoxy-1-[2-(4-methylpiperazin-1-yl)ethyl]-1H-indol-3-yl}methylene)-6-hydroxy-4-methyl-1-benzofuran-3(2H)-one;
(2Z)-2-({1-[3-(dimethylamino)propyl]-5-methoxy-2-methyl-1H-indol-3-yl}methylene)-4-fluoro-6-hydroxy-1-benzofuran-3(2H)-one;
(2Z)-4-chloro-2-({1-[3-(dimethylamino)propyl]-5-methoxy-2-methyl-1H-indol-3-yl}methylene)-6-hydroxy-1-benzofuran-3(2H)-one;
(2Z)-4-chloro-6-hydroxy-2-({5-methoxy-2-methyl-1-[2-(4-methylpiperazin-1-yl)ethyl]-1H-indol-3-yl}methylene)-1-benzofuran-3(2H)-one;
(2Z)-2-({1-[3-(dimethylamino)propyl]-5-methoxy-2-methyl-1H-indol-3-yl}methylene)-6-hydroxy-4-methyl-1-benzofuran-3(2H)-one;
(2Z)-2-({2-(3,5-dimethylisoxazol-4-yl)-5-methoxy-1-[2-(4-methylpiperazin-1-yl)ethyl]-1H-indol-3-yl}methylene)-6-hydroxy-1-benzofuran-3(2H)-one;
(2Z)-2-{[1-(2-azepan-1-ylethyl)-5-methoxy-2-methyl-1H-indol-3-yl]methylene}-6-hydroxy-1-benzofuran-3(2H)-one;
4-{3-[(4,6-dihydroxy-3-oxo-1-benzofuran-2(3H)-ylidene)methyl]-7-ethyl-5-methoxy-1H-indol-1-yl}butanenitrile;
(2Z)-6-hydroxy-4-methoxy-2-[(5-methoxy-1H-indol-3-yl)methylene]-1-benzofuran-3(2H)-one;
(2Z)-2-({7-ethyl-5-methoxy-1-[2-(4-methylpiperazin-1-yl)ethyl]-1H-indol-3-yl}methylene)-6-hydroxy-1-benzofuran-3(2H)-one;
4-{7-ethyl-3-[(Z)-(6-hydroxy-3-oxo-1-benzofuran-2(3H)-ylidene)methyl]-5-methoxy-1H-indol-1-yl}butanenitrile;
4-{7-ethyl-3-[(Z)-(4-hydroxy-3-oxo-1-benzofuran-2(3H)-ylidene)methyl]-5-methoxy-1H-indol-1-yl}butanenitrile;
(2Z)-2-[(1,4-dimethyl-2-phenyl-1H-indol-3-yl)methylene]-4,6-dihydroxy-1-benzofuran-3(2H)-one;
(2Z)-2-[(1,4-dimethyl-2-pyridin-2-yl-1H-indol-3-yl)methylene]-4,6-dihydroxy-1-benzofuran-3(2H)-one;
(2Z)-4,6-dihydroxy-2-{[1-(2-hydroxyethyl)-4-phenyl-1H-indol-3-yl]methylene}-1-benzofuran-3(2H)-one;
(2Z)-6-hydroxy-2-[(5-methoxy-7-pyridin-4-yl-1H-indol-3-yl)methylene]-1-benzofuran-3(2H)-one;
(2Z)-6-hydroxy-2-[(5-methoxy-7-pyridin-3-yl-1H-indol-3-yl)methylene]-1-benzofuran-3(2H)-one;
(2Z)-2-[(5-bromo-1H-indol-3-yl)methylene]-7-methoxy-1-benzofuran-3(2H)-one;
7-hydroxy-2-[(5-methoxy-1H-indol-3-yl)methylene]-1-benzofuran-3(2H)-one;
7-methoxy-2-[(5-methoxy-1H-indol-3-yl)methylene]-1-benzofuran-3(2H)-one;
1-{(2Z)-2-[(5-methoxy-1H-indol-3-yl)methylene]-3-oxo-2,3-dihydro-1-benzofuran-5-yl}-3-pyridin-3-ylurea;
1-{(2Z)-2-[(5-methoxy-1H-indol-3-yl)methylene]-3-oxo-2,3-dihydro-1-benzofuran-5-yl}-3-phenylurea;
1-isopropyl-3-{(2Z)-2-[(5-methoxy-1H-indol-3-yl)methylene]-3-oxo-2,3-dihydro-1-benzofuran-5-yl}urea;
1-butyl-3-{(2Z)-2-[(5-methoxy-1H-indol-3-yl)methylene]-3-oxo-2,3-dihydro-1-benzofuran-5-yl}urea;
1-cyclohexyl-3-{(2Z)-2-[(5-methoxy-1H-indol-3-yl)methylene]-3-oxo-2,3-dihydro-1-benzofuran-5-yl}urea;
1-ethyl-3-{(2Z)-2-[(5-methoxy-1H-indol-3-yl)methylene]-3-oxo-2,3-dihydro-1-benzofuran-5-yl}urea;
methyl ({(2Z)-2-[(5-methoxy-1H-indol-3-yl)methylene]-3-oxo-2,3-dihydro-1-benzofuran-5-yl}carbamoyl)carbamate;
1-{(2Z)-2-[(5-methoxy-1H-indol-3-yl)methylene]-3-oxo-2,3-dihydro-1-benzofuran-5-yl}-3-(4-methoxyphenyl)urea;
1-[4-(dimethylamino)phenyl]-3-{(2Z)-2-[(5-methoxy-1H-indol-3-yl)methylene]-3-oxo-2,3-dihydro-1-benzofuran-5-yl}urea;
4-{3-[(Z)-(4,6-dihydroxy-3-oxo-1-benzofuran-2(3H)-ylidene)methyl]-2-ethyl-5-methoxy-1H-indol-1-yl}butanenitrile;
2-[(5-methoxy-1H-indol-3-yl)methylene]-3-oxo-2,3-dihydro-1-benzofuran-6-yl trifluoromethanesulfonate;
2-[(5-methoxy-1H-indol-3-yl)methylene]-3-oxo-2,3-dihydro-1-benzofuran-6-carboxamide;
(2Z)-4,6-dihydroxy-2-[(1-methyl-4-morpholin-4-yl-1H-indol-3-yl)methylene]-1-benzofuran-3(2H)-one;
(2Z)-4,6-dihydroxy-2-[(4-methoxy-1-methyl-2-phenyl-1H-indol-3-yl)methylene]-1-benzofuran-3(2H)-one;
(2Z)-6-hydroxy-2-[(5-methoxy-7-pyrimidin-5-yl-1H-indol-3-yl)methylene]-1-benzofuran-3(2H)-one;
(2Z)-4,6-dihydroxy-2-[(1-methyl-4-nitro-2-phenyl-1H-indol-3-yl)methylene]-1-benzofuran-3(2H)-one;
4-{3-[(Z)-(6-hydroxy-3-oxo-1-benzofuran-2(3H)-ylidene)methyl]-5-methoxy-7-pyridin-4-yl-1H-indol-1-yl}butanenitrile;
4-{3-[(Z)-(6-hydroxy-3-oxo-1-benzofuran-2(3H)-ylidene)methyl]-5-methoxy-7-pyrimidin-5-yl-1H-indol-1-yl}butanenitrile;
4-{3-[(Z)-(6-hydroxy-3-oxo-1-benzofuran-2(3H)-ylidene)methyl]-5-methoxy-7-pyridin-3-yl-1H-indol-1-yl}butanenitrile;
4-{3-[(Z)-(4,6-dihydroxy-3-oxo-1-benzofuran-2(3H)-ylidene)methyl]-5-methoxy-7-pyrimidin-5-yl-1H-indol-1-yl}butanenitrile;
(2Z)-2-[(5-methoxy-1H-indol-3-yl)methylene]-3-oxo-2,3-dihydro-1-benzofuran-6-carbonitrile;
(2Z)-2-[(5-methoxy-1H-indol-3-yl)methylene]-6-(2H-tetrazol-5-yl)-1-benzofuran-3(2H)-one;
4-{3-[(Z)-(4,6-dihydroxy-3-oxo-1-benzofuran-2(3H)-ylidene)methyl]-5-methoxy-1Hindol-1-yl}butanenitrile;
4-{3-[(Z)-(6-hydroxy-3-oxo-1-benzofuran-2(3H)-ylidene)methyl]-5-methoxy-1Hindol-1-yl}butanenitrile;
(2Z)-2-({7-ethyl-5-methoxy-1-[2-(4-methylpiperazin-1-yl)ethyl]-1H-indol-3-yl}methylene)-4,6-dihydroxy-1-benzofuran-3(2H)-one;
4-{3-[(Z)-(5-hydroxy-3-oxo-1-benzofuran-2(3H)-ylidene)methyl]-5-methoxy-1Hindol-1-yl}butanenitrile;
(2Z)-2-({1-[3-(dimethylamino)propyl]-5-methoxy-1H-indol-3-yl}methylene)-5-hydroxy-1-benzofuran-3(2H)-one;
(2Z)-5-bromo-2-({1-[3-(dimethylamino)propyl]-5-methoxy-1H-indol-3-yl}methylene)-1-benzofuran-3(2H)-one;
2Z)-5-hydroxy-2-({5-methoxy-2-methyl-1-[2-(4-methylpiperazin-1-yl)ethyl]-1H-indol-3-yl}methylene)-1-benzofuran-3(2H)-one;
1-[(2Z)-2-({1-[3-(dimethylamino)propyl]-5-methoxy-1H-indol-3-yl}methylene)-3-oxo-2,3-dihydro-1-benzofuran-5-yl]-3-methylurea;
(2Z)-5-bromo-2-[(5-methoxy-1H-indol-3-yl)methylene]-1-benzofuran-3(2H)-one;
30 (2Z)-2-[(5-methoxy-1H-indol-3-yl)methylene]-3-oxo-2,3-dihydro-1-benzofuran-5-carbonitrile;
(2Z)-2-[(5-methoxy-1H-indol-3-yl)methylene]-5-(1H-tetrazol-5-yl)-1-benzofuran-3(2H)-one;
N-[(2Z)-2-({1-[3-(dimethylamino)propyl]-5-methoxy-1H-indol-3-yl}methylene)-3-oxo-2,3-dihydro-1-benzofuran-5-yl]acetamide;
(2Z)-5-bromo-2-[(1-methyl-4-phenyl-1H-indol-3-yl)methylene]-1-benzofuran-3(2H)-one;
1-methyl-3-{(2Z)-2-[(1-methyl-4-phenyl-1H-indol-3-yl)methylene]-3-oxo-2,3-dihydro-1-benzofuran-5-yl}urea;
(2Z)-5,6-dihydroxy-2-[(5-methoxy-1H-indol-3-yl)methylene]-1-benzofuran-3(2H)-one;
(2Z)-5-hydroxy-2-[(1-methyl-4-phenyl-1H-indol-3-yl)methylene]-1-benzofuran-3(2H)-one;
(2Z)-4,6-dihydroxy-2-[(1-methyl-4-phenyl-1H-indol-3-yl)methylene]-1-benzofuran-3(2H)-one;
tert-butyl {(2Z)-2-[(1-methyl-4-phenyl-1H-indol-3-yl)methylene]-3-oxo-2,3-dihydro-1-benzofuran-5-yl}carbamate;
1-[(2Z)-2-({2-cyclohexyl-5-methoxy-1-[2-(4-methylpiperazin-1-yl)ethyl]-1H-indol-3-yl}methylene)-3-oxo-2,3-dihydro-1-benzofuran-5-yl]-3-methylurea;
(2Z)-5-amino-2-[(1-methyl-4-phenyl-1H-indol-3-yl)methylene]-1-benzofuran-3(2H)-one;
1-{(2Z)-2-[(1-methyl-4-phenyl-1H-indol-3-yl)methylene]-3-oxo-2,3-dihydro-1-benzofuran-5-yl}-3-pyridin-3-ylurea;
1-{(2Z)-2-[(5-methoxy-1H-indol-3-yl)methylene]-3-oxo-2,3-dihydro-1-benzofuran-5-yl}-3-methylurea;
1-[(2Z)-2-({1-[3-(dimethylamino)propyl]-5-methoxy-1H-indol-3-yl}methylene)-3-oxo-2,3-dihydro-1-benzofuran-5-yl]urea;
methyl [(2Z)-2-({1-[3-(dimethylamino)propyl]-5-methoxy-1H-indol-3-yl}methylene)-3-oxo-2,3-dihydro-1-benzofuran-5-yl]carbamate;
(2Z)-2-({1-[3-(dimethylamino)propyl]-5-methoxy-1H-indol-3-yl}methylene)-Nmethyl-3-oxo-2,3-dihydro-1-benzofuran-5-carboxamide;
1-[(2Z)-2-({1-[3-(dimethylamino)propyl]-5-methoxy-1H-indol-3-yl}methylene)-3-oxo-2,3-dihydro-1-benzofuran-5-yl]-3-(4-methoxyphenyl)urea;
(2Z)-5-(hydroxymethyl)-2-({5-methoxy-1-[2-(4-methylpiperazin-1-yl)ethyl]-1Hindol-3-yl}methylene)-1-benzofuran-3(2H)-one;
1-ethyl-3-{(2Z)-2-[(1-methyl-4-phenyl-1H-indol-3-yl)methylidene]-3-oxo-2,3-dihydro-1-benzofuran-5-yl}urea;
(2E)-4,6-dihydroxy-2-[(1-methyl-4-phenyl-1H-indol-3-yl)methylene]-1-benzofuran-3(2H)-one-(2Z)-4,6-dihydroxy-2-[(1-methyl-4-phenyl-1H-indol-3-yl)methylene]-1-benzofuran-3(2H)-one (1:1);
(2Z)-2-({5-methoxy-1-[2-(4-methylpiperazin-1-yl)ethyl]-1H-indol-3-yl}methylene)-3-oxo-2,3-dihydro-1-benzofuran-5-yl methylcarbamate;
1-[(2Z)-2-({1-[3-(dimethylamino)propyl]-5-methoxy-1H-indol-3-yl}methylene)-3-oxo-2,3-dihydro-1-benzofuran-5-yl]-3-ethylurea;
1-[(2Z)-2-({5-methoxy-1-[2-(4-methylpiperazin-1-yl)ethyl]-1H-indol-3-yl}methylene)-3-oxo-2,3-dihydro-1-benzofuran-5-yl]-3-prop-2-yn-1-ylurea;
1-(2-aminoethyl)-3-[(2Z)-2-({5-methoxy-1-[2-(4-methylpiperazin-1-yl)ethyl]-1Hindol-3-yl}methylene)-3-oxo-2,3-dihydro-1-benzofuran-5-yl]urea;
1-allyl-3-[(2Z)-2-({5-methoxy-1-[2-(4-methylpiperazin-1-yl)ethyl]-1H-indol-3-yl}methylene)-3-oxo-2,3-dihydro-1-benzofuran-5-yl]urea;
1-[(2Z)-2-({5-methoxy-1-[2-(4-methylpiperazin-1-yl)ethyl]-1H-indol-3-yl}methylene)-3-oxo-2,3-dihydro-1-benzofuran-5-yl]urea;
1-azetidin-3-yl-3-[(2Z)-2-({5-methoxy-1-[2-(4-methylpiperazin-1-yl)ethyl]-1Hindol-3-yl}methylene)-3-oxo-2,3-dihydro-1-benzofuran-5-yl]urea;
1-[(2Z)-2-({5-methoxy-1-[2-(4-methylpiperazin-1-yl)ethyl]-1H-indol-3-yl}methylene)-3-oxo-2,3-dihydro-1-benzofuran-5-yl]-3-methylurea;
1-[(2Z)-2-({1-[3-(dimethylamino)propyl]-5-methoxy-1H-indol-3-yl}methylene)-3-oxo-2,3-dihydro-1-benzofuran-5-yl]-3-(2-hydroxyethyl)urea;
1-[(2Z)-2-({5-methoxy-2-methyl-1-[2-(4-methylpiperazin-1-yl)ethyl]-1H-indol-3-yl}methylene)-3-oxo-2,3-dihydro-1-benzofuran-5-yl]-3-methylurea;
(2E)-4,6-dihydroxy-2-[(1-methyl-4-phenyl-1H-indol-3-yl)methylene]-1-benzofuran-3(2H)-one;
(2Z)-2-({5-methoxy-2-methyl-1-[2-(4-methylpiperazin-1-yl)ethyl]-1H-indol-3-yl}methylene)-3-oxo-2,3-dihydro-1-benzofuran-6-yl methyl(phenyl)carbamate;
1-[(2Z)-2-({2-cyclopropyl-5-methoxy-1-[2-(4-methylpiperazin-1-yl)ethyl]-1Hindol-3-yl}methylene)-3-oxo-2,3-dihydro-1-benzofuran-5-yl]-3-methylurea;
1-cyclopropyl-3-[(2Z)-2-({1-[3-(dimethylamino)propyl]-5-methoxy-1H-indol-3-yl}methylene)-3-oxo-2,3-dihydro-1-benzofuran-5-yl]urea;
1-[(2Z)-2-({2-cyclopentyl-5-methoxy-1-[2-(4-methylpiperazin-1-yl)ethyl]-1Hindol-3-yl}methylene)-3-oxo-2,3-dihydro-1-benzofuran-5-yl]-3-methylurea;
1-[(2Z)-2-({2-cyclobutyl-5-methoxy-1-[2-(4-methylpiperazin-1-yl)ethyl]-1H-indol-3-yl}methylene)-3-oxo-2,3-dihydro-1-benzofuran-5-yl]-3-methylurea;
1-[(2Z)-2-({5-methoxy-1-[2-(4-methylpiperazin-1-yl)ethyl]-1H-indol-3-yl}methylene)-3-oxo-2,3-dihydro-1-benzofuran-5-yl]-3-[2-(methylamino)ethyl]urea;
1-[(2Z)-2-({5-methoxy-1-[2-(4-methylpiperazin-1-yl)ethyl]-1H-indol-3-yl}methylene)-3-oxo-2,3-dihydro-1-benzofuran-5-yl]-3-[3-(methylamino)propyl]urea;
1-(4-aminobutyl)-3-[(2Z)-2-({5-methoxy-1-[2-(4-methylpiperazin-1-yl)ethyl]-1Hindol-3-yl}methylene)-3-oxo-2,3-dihydro-1-benzofuran-5-yl]urea;
1-(3-aminopropyl)-3-[(2Z)-2-({5-methoxy-1-[2-(4-methylpiperazin-1-yl)ethyl]-1Hindol-3-yl}methylene)-3-oxo-2,3-dihydro-1-benzofuran-5-yl]urea;
1-[(2Z)-2-({5-methoxy-7-[(1E)-3-morpholin-4-ylprop-1-en-1-yl]-1H-indol-3-yl}methylene)-3-oxo-2,3-dihydro-1-benzofuran-5-yl]-3-methylurea;
1-[(2Z)-2-({1-[3-(dimethylamino)propyl]-5-methoxy-1H-indol-3-yl}methylene)-3-oxo-2,3-dihydro-1-benzofuran-5-yl]-3-(3-hydroxypropyl)urea;
1-[3-(dimethylamino)propyl]-3-[(2Z)-2-({5-methoxy-1-[2-(4-methylpiperazin-1-yl)ethyl]-1H-indol-3-yl}methylene)-3-oxo-2,3-dihydro-1-benzofuran-5-yl]urea;
1-[(2Z)-2-{[5-methoxy-7-(morpholin-4-ylmethyl)-1H-indol-3-yl]methylene}-3-oxo-2,3-dihydro-1-benzofuran-5-yl]-3-methylurea;
1-[(2Z)-2-({5-methoxy-7-[(4-methylpiperazin-1-yl)methyl]-1H-indol-3-yl}methylene)-3-oxo-2,3-dihydro-1-benzofuran-5-yl]-3-methylurea;
1-[(2Z)-2-({5-methoxy-1-methyl-7-[3-(4-methylpiperazin-1-yl)propyl]-1H-indol-3-yl}methylene)-3-oxo-2,3-dihydro-1-benzofuran-5-yl]-3-methylurea;
1-[(2Z)-2-({7-[(1E)-3-(dimethylamino)prop-1-en-1-yl]-5-methoxy-1H-indol-3-yl}methylene)-3-oxo-2,3-dihydro-1-benzofuran-5-yl]-3-methylurea;
1-[2-(dimethylamino)ethyl]-3-[(2Z)-2-({5-methoxy-1-[2-(4-methylpiperazin-1-yl)ethyl]-1H-indol-3-yl}methylene)-3-oxo-2,3-dihydro-1-benzofuran-5-yl]urea;
1-[(2Z)-2-({5-methoxy-1-[2-(4-methylpiperazin-1-yl)ethyl]-2-phenyl-1H-indol-3-yl}methylene)-3-oxo-2,3-dihydro-1-benzofuran-5-yl]-3-methylurea;
1-(2-aminoethyl)-3-[(2Z)-2-({5-methoxy-1-[2-(4-methylpiperazin-1-yl)ethyl]-2-phenyl-1H-indol-3-yl}methylene)-3-oxo-2,3-dihydro-1-benzofuran-5-yl]urea;
1-(2-aminoethyl)-3-[(2Z)-2-({2-cyclopentyl-5-methoxy-1-[2-(4-methylpiperazin-1-yl)ethyl]-1H-indol-3-yl}methylene)-3-oxo-2,3-dihydro-1-benzofuran-5-yl]urea;
1-[(2Z)-2-{[5-methoxy-1-(3-piperidin-1-ylpropyl)-1H-indol-3-yl]methylene}-3-oxo-2,3-dihydro-1-benzofuran-5-yl]-3-methylurea;
1-[(2Z)-2-{[1-(3-cyanopropyl)-5-methoxy-1H-indol-3-yl]methylene}-3-oxo-2,3-dihydro-1-benzofuran-5-yl]-3-methylurea;
1-[(2Z)-2-{[5-methoxy-1-(3-morpholin-4-ylpropyl)-1H-indol-3-yl]methylene}-3-oxo-2,3-dihydro-1-benzofuran-5-yl]-3-methylurea;
1-[(2Z)-2-{[2-(3,5-dimethylisoxazol-4-yl)-5-methoxy-1H-indol-3-yl]methylene}-3-oxo-2,3-dihydro-1-benzofuran-5-yl]-3-methylurea;
1-[(2Z)-2-({5-methoxy-7-[3-(4-methylpiperazin-1-yl)propyl]-1H-indol-3-yl}methylene)-3-oxo-2,3-dihydro-1-benzofuran-5-yl]-3-methylurea;
1-[(2Z)-2-({2-cyclohexyl-5-methoxy-1-[2-(4-methylpiperazin-1-yl)ethyl]-1H-indol-3-yl}methylene)-3-oxo-2,3-dihydro-1-benzofuran-5-yl]-3-[2-(methylamino)ethyl]urea;
1-[(2Z)-2-{[5-methoxy-2-methyl-1-(3-morpholin-4-ylpropyl)-1H-indol-3-yl]methylene}-3-oxo-2,3-dihydro-1-benzofuran-5-yl]-3-methylurea;
1-[(2Z)-2-({5-methoxy-2-methyl-1-[3-(4-methylpiperazin-1-yl)propyl]-1H-indol-3-yl}methylene)-3-oxo-2,3-dihydro-1-benzofuran-5-yl]-3-methylurea;
1-[(2Z)-2-({1-[3-(4-hydroxypiperidin-1-yl)propyl]-5-methoxy-2-methyl-1H-indol-3-yl}methylene)-3-oxo-2,3-dihydro-1-benzofuran-5-yl]-3-methylurea;
1-[(2Z)-2-{[5-methoxy-2-methyl-1-(3-piperidin-1-ylpropyl)-1H-indol-3-yl]methylene}-3-oxo-2,3-dihydro-1-benzofuran-5-yl]-3-methylurea;
1-[(2Z)-2-({1-[3-(3-hydroxypyrrolidin-1-yl)propyl]-5-methoxy-2-methyl-1H-indol-3-yl}methylene)-3-oxo-2,3-dihydro-1-benzofuran-5-yl]-3-methylurea;
1-[(2Z)-2-({5-methoxy-1-[2-(4-methylpiperazin-1-yl)ethyl]-2-pyridin-4-yl-1Hindol-3-yl}methylene)-3-oxo-2,3-dihydro-1-benzofuran-5-yl]-3-methylurea;
1-[(2Z)-2-{[2-(3-isopropyl-1,2,4-oxadiazol-5-yl)-5-methoxy-1H-indol-3-yl]methylene}-3-oxo-2,3-dihydro-1-benzofuran-5-yl]-3-methylurea;
1-[(2Z)-2-{[2-(3-cyclopropyl-1,2,4-oxadiazol-5-yl)-5-methoxy-1H-indol-3-yl]methylene}-3-oxo-2,3-dihydro-1-benzofuran-5-yl]-3-methylurea;
1-[(2Z)-2-{[5-methoxy-2-(3-propyl-1,2,4-oxadiazol-5-yl)-1H-indol-3-yl]methylene}-3-oxo-2,3-dihydro-1-benzofuran-5-yl]-3-methylurea;
1-[(2Z)-2-({5-methoxy-7-[(1E)-3-(4-methylpiperazin-1-yl)prop-1-en-1-yl]-1Hindol-3-yl}methylene)-3-oxo-2,3-dihydro-1-benzofuran-5-yl]-3-methylurea;
1-{(2Z)-2-[(7-cyano-5-methoxy-1H-indol-3-yl)methylene]-3-oxo-2,3-dihydro-1-benzofuran-5-yl}-3-methylurea;
1-[(2Z)-2-({5-methoxy-1-[2-(4-methylpiperazin-1-yl)ethyl]-2-pyridin-3-yl-1Hindol-3-yl}methylene)-3-oxo-2,3-dihydro-1-benzofuran-5-yl]-3-methylurea;
1-[(2Z)-2-{[5-methoxy-2-methyl-1-(3-pyrrolidin-1-ylpropyl)-1H-indol-3-yl]methylidene}-3-oxo-2,3-dihydro-1-benzofuran-5-yl]-3-methylurea;
1-[(2Z)-2-({1-[3-(1H-imidazol-1-yl)propyl]-5-methoxy-2-methyl-1H-indol-3-yl}methylidene)-3-oxo-2,3-dihydro-1-benzofuran-5-yl]-3-methylurea;
1-{(2Z)-2-[(1-{3-[4-(2-hydroxyethyl)piperazin-1-yl]propyl}-5-methoxy-2-methyl-1H-indol-3-yl)methylidene]-3-oxo-2,3-dihydro-1-benzofuran-5-yl}-3-methylurea;
5-methoxy-N,N-dimethyl-3-[(Z)-{5-[(methylcarbamoyl)amino]-3-oxo-1-benzofuran-2(3H)-ylidene}methyl]-1H-indole-2-carboxamide;
1-[(2Z)-2-({5-methoxy-2-[(4-methylpiperazin-1-yl)carbonyl]-1H-indol-3-yl}methylidene)-3-oxo-2,3-dihydro-1-benzofuran-5-yl]-3-methylurea;
N-[2-(dimethylamino)ethyl]-5-methoxy-N-methyl-3-[(Z)-{5-[(methylcarbamoyl)amino]-3-oxo-1-benzofuran-2(3H)-ylidene}methyl]-1H-indole-2-carboxamide;
5-methoxy-N-methyl-3-[(Z)-{5-[(methylcarbamoyl)amino]-3-oxo-1-benzofuran-2(3H)-ylidene}methyl]-1H-indole-2-carboxamide;
1-{(2Z)-2-[(2-cyano-5-methoxy-1H-indol-3-yl)methylidene]-3-oxo-2,3-dihydro-1-benzofuran-5-yl}-3-methylurea;
N-[2-(dimethylamino)ethyl]-5-methoxy-3-[(Z)-{5-[(methylcarbamoyl)amino]-3-oxo-1-benzofuran-2(3H)-ylidene}methyl]-1H-indole-2-carboxamide;
1-[(2Z)-2-{[5-methoxy-2-(1,2,3,6-tetrahydropyridin-4-yl)-1H-indol-3-yl]methylidene}-3-oxo-2,3-dihydro-1-benzofuran-5-yl]-3-methylurea;
1-[(2Z)-2-({5-methoxy-1-[2-(4-methylpiperazin-1-yl)ethyl]-2-(1,2,3,6-tetrahydropyridin-4-yl)-1H-indol-3-yl}methylidene)-3-oxo-2,3-dihydro-1-benzofuran-5-yl]-3-methylurea;
1-[(2Z)-2-{[5-methoxy-2-methyl-1-(2-morpholin-4-ylethyl)-1H-indol-3-yl]methylidene}-3-oxo-2,3-dihydro-1-benzofuran-5-yl]-3-methylurea;
1-[(2Z)-2-{[5-methoxy-2-methyl-1-(2-piperidin-1-ylethyl)-1H-indol-3-yl]methylidene}-3-oxo-2,3-dihydro-1-benzofuran-5-yl]-3-methylurea;
1-[(2Z)-2-({1-[2-(dimethylamino)ethyl]-5-methoxy-2-methyl-1H-indol-3-yl}methylidene)-3-oxo-2,3-dihydro-1-benzofuran-5-yl]-3-methylurea;
1-[(2Z)-2-{[5-methoxy-2-methyl-1-(2-pyrrolidin-1-ylethyl)-1H-indol-3-yl]methylidene}-3-oxo-2,3-dihydro-1-benzofuran-5-yl]-3-methylurea;
1-[(2Z)-2-({2-[(dimethylamino)methyl]-5-methoxy-1H-indol-3-yl}methylidene)-3-oxo-2,3-dihydro-1-benzofuran-5-yl]-3-methylurea;
1-[(2Z)-2-{[2-([2-(dimethylamino)ethyl](methyl)aminomethyl)-5-methoxy-1Hindol-3-yl]methylidene}-3-oxo-2,3-dihydro-1-benzofuran-5-yl]-3-methylurea;
1-[(2Z)-2-({5-methoxy-2-[3-(1-methylethyl)-1,2,4-oxadiazol-5-yl]-1-[2-(4-methylpiperazin-1-yl)ethyl]-1H-indol-3-yl}methylidene)-3-oxo-2,3-dihydro-1-benzofuran-5-yl]-3-methylurea;
1-[(2Z)-2-({5-methoxy-2-[(4-methylpiperazin-1-yl)methyl]-1H-indol-3-yl}methylidene)-3-oxo-2,3-dihydro-1-benzofuran-5-yl]-3-methylurea;
1-[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]-3-methylurea;
1-[(2Z)-2-({5-methoxy-2-methyl-1-[2-(4-methylpiperazin-1-yl)ethyl]-1H-indol-3-yl}methylidene)-3-oxo-2,3-dihydro-1-benzofuran-5-yl]-3-pyridin-3-ylurea;
1-[(2Z)-2-({5-methoxy-2-methyl-1-[2-(4-methylpiperazin-1-yl)ethyl]-1H-indol-3-yl}methylene)-3-oxo-2,3-dihydro-1-benzofuran-5-yl]-3-{4-[(4-methylpiperazin-1-yl)carbonyl]phenyl}urea;
1-[(2Z)-2-{[1-(2-hydroxyethyl)-5-methoxy-2-methyl-1H-indol-3-yl]methylene}-3-oxo-2,3-dihydro-1-benzofuran-5-yl]-3-{4-[(4-methylpiperazin-1-yl)carbonyl]phenyl}urea;
1-{(2Z)-2-[(1-ethyl-5-methoxy-2-methyl-1H-indol-3-yl)methylene]-3-oxo-2,3-dihydro-1-benzofuran-5-yl}-3-{4-[(4-methylpiperazin-1-yl)carbonyl]phenyl}urea;
1-[(2Z)-2-({5-methoxy-2-[3-(1-methylethyl)-1,2,4-oxadiazol-5-yl]-1-[2-(4-methylpiperazin-1-yl)ethyl]-1H-indol-3-yl}methylidene)-3-oxo-2,3-dihydro-1-benzofuran-5-yl]-3-methylurea;
N-(2-hydroxy-1,1-dimethylethyl)-5-methoxy-3-[(Z)-{5-[(methylcarbamoyl)amino]-3-oxo-1-benzofuran-2(3H)-ylidene}methyl]-1H-indole-2-carboxamide;
1-[(2Z)-2-({2-(3,5-dimethylisoxazol-4-yl)-5-methoxy-1-[2-(4-methylpiperazin-1-yl)ethyl]-1H-indol-3-yl}methylidene)-3-oxo-2,3-dihydro-1-benzofuran-5-yl]-3-methylurea;
1-{(2Z)-2-[(2-{4-[(dimethylamino)methyl]phenyl}-5-methoxy-1H-indol-3-yl)methylidene]-3-oxo-2,3-dihydro-1-benzofuran-5-yl}-3-methylurea;
1-[(2Z)-2-{[2-(3,5-dimethyl-1H-pyrazol-4-yl)-5-methoxy-1H-indol-3-yl]methylidene}-3-oxo-2,3-dihydro-1-benzofuran-5-yl]-3-methylurea;
1-[(2Z)-2-({2-(3-cyclopropyl-1,2,4-oxadiazol-5-yl)-5-methoxy-1-[2-(4-methylpiperazin-1-yl)ethyl]-1H-indol-3-yl}methylidene)-3-oxo-2,3-dihydro-1-benzofuran-5-yl]-3-methylurea;
1-[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]-3-methylurea;
1-[(2Z)-2-({2-cyano-5-methoxy-1-[2-(4-methylpiperazin-1-yl)ethyl]-1H-indol-3-yl}methylidene)-3-oxo-2,3-dihydro-1-benzofuran-5-yl]-3-methylurea;
1-{(2Z)-2-[(2-{3-[(dimethylamino)methyl]phenyl}-5-methoxy-1H-indol-3-yl)methylidene]-3-oxo-2,3-dihydro-1-benzofuran-5-yl}-3-methylurea;
1-{(2Z)-2-[(2-{4-[(dimethylamino)methyl]phenyl}-5-methoxy-1-[2-(4-methylpiperazin-1-yl)ethyl]-1H-indol-3-yl)methylidene]-3-oxo-2,3-dihydro-1-benzofuran-5-yl}-3-methylurea;
1-{(2Z)-2-[(2-tert-butyl-5-methoxy-1H-indol-3-yl)methylidene]-3-oxo-2,3-dihydro-1-benzofuran-5-yl}-3-methylurea;
1-[(2Z)-2-({5-methoxy-2-methyl-1-[2-(4-methylpiperazin-1-yl)ethyl]-1H-indol-3-yl}methylidene)-3-oxo-2,3-dihydro-1-benzofuran-5-yl]-3-pyridin-3-ylurea;
1-[4-(dimethylamino)phenyl]-3-[(2Z)-2-({5-methoxy-2-methyl-1-[2-(4-methylpiperazin-1-yl)ethyl]-1H-indol-3-yl}methylidene)-3-oxo-2,3-dihydro-1-benzofuran-5-yl]urea;
1-[(2Z)-2-{[1-(3-cyanopropyl)-5-methoxy-2-(1,3,5-trimethyl-1H-pyrazol-4-yl)-1H-indol-3-yl]methylene}-3-oxo-2,3-dihydro-1-benzofuran-5-yl]-3-methylurea;
1-[(2Z)-2-{[5-methoxy-2-(1,3,5-trimethyl-1H-pyrazol-4-yl)-1H-indol-3-yl]methylene}-3-oxo-2,3-dihydro-1-benzofuran-5-yl]-3-pyridin-3-ylurea;
1-[(2Z)-2-({5-methoxy-2-(4-methylpiperazin-1-yl)-1-[2-(4-methylpiperazin-1-yl)ethyl]-1H-indol-3-yl}methylene)-3-oxo-2,3-dihydro-1-benzofuran-5-yl]-3-methylurea;
1-[(2Z)-2-{[2-(2,6-dimethoxyphenyl)-5-methoxy-1H-indol-3-yl]methylene}-3-oxo-2,3-dihydro-1-benzofuran-5-yl]-3-methylurea;
1-[(2Z)-2-({2-cyclopentyl-5-methoxy-1-[2-(4-methylpiperazin-1-yl)ethyl]-1H-indol-3-yl}methylene)-3-oxo-2,3-dihydro-1-benzofuran-5-yl]-3-pyridin-3-ylurea;
N-[2-(dimethylamino)ethyl]-4-[({(2Z)-2-[(1-ethyl-5-methoxy-2-methyl-1H-indol-3-yl)methylene]-3-oxo-2,3-dihydro-1-benzofuran-5-yl}carbamoyl)amino]-N-methylbenzamide;
1-[(2Z)-2-({5-methoxy-2-methyl-1-[2-(4-methylpiperazin-1-yl)ethyl]-1H-indol-3-yl}methylene)-6-methyl-3-oxo-2,3-dihydro-1-benzofuran-5-yl]-3-methylurea;
1-{(2Z)-2-[(2-cyclohexyl-5-methoxy-1H-indol-3-yl)methylene]-3-oxo-2,3-dihydro-1-benzofuran-5-yl}-3-methylurea;
1-{(2Z)-2-[(1-ethyl-5-methoxy-1H-indol-3-yl)methylene]-3-oxo-2,3-dihydro-1-benzofuran-5-yl}-3-{4-[(4-methylpiperazin-1-yl)carbonyl]phenyl}urea;
1-{(2Z)-2-[(5-methoxy-1H-indol-3-yl)methylene]-3-oxo-2,3-dihydro-1-benzofuran-5-yl}-3-{4-[(4-methylpiperazin-1-yl)carbonyl]phenyl}urea;
1-{(2Z)-2-[(2-cyclohexyl-5-methoxy-1H-indol-3-yl)methylene]-3-oxo-2,3-dihydro-1-benzofuran-5-yl}-3-{4-[(4-methylpiperazin-1-yl)carbonyl]phenyl}urea;
1-{(2Z)-2-[(2-cyclohexyl-1-ethyl-5-methoxy-1H-indol-3-yl)methylene]-3-oxo-2,3-dihydro-1-benzofuran-5-yl}-3-{4-[(4-methylpiperazin-1-yl)carbonyl]phenyl}urea;
1-[(2Z)-2-({5-methoxy-1-[2-(4-methylpiperazin-1-yl)ethyl]-2-(1,3,5-trimethyl-1H-pyrazol-4-yl)-1H-indol-3-yl}methylene)-3-oxo-2,3-dihydro-1-benzofuran-5-yl]-3-methylurea;
1-[(2Z)-2-({5-methoxy-2-methyl-1-[2-(4-methylpiperazin-1-yl)ethyl]-1H-indol-3-yl}methylene)-3-oxo-2,3-dihydro-1-benzofuran-5-yl]-3-methylthiourea;
1-[(2Z)-2-{[1-{2-[(2-hydroxyethyl)amino]ethyl}-5-methoxy-2-(1,3,5-trimethyl-1H-pyrazol-4-yl)-1H-indol-3-yl]methylene}-3-oxo-2,3-dihydro-1-benzofuran-5-yl]-3-methylurea;
N-[2-(dimethylamino)ethyl]-4-({[(2Z)-2-({5-methoxy-2-methyl-1-[2-(4-methylpiperazin-1-yl)ethyl]-1H-indol-3-yl}methylene)-3-oxo-2,3-dihydro-1-benzofuran-5-yl]carbamoyl}amino)-N-methylbenzamide;
1-(4-{[4-(dimethylamino)piperidin-1-yl]carbonyl}phenyl)-3-{(2Z)-2-[(1-ethyl-5-methoxy-2-methyl-1H-indol-3-yl)methylene]-3-oxo-2,3-dihydro-1-benzofuran-5-yl}urea;
1-{(2Z)-2-[(1-ethyl-5-methoxy-2-methyl-1H-indol-3-yl)methylene]-3-oxo-2,3-dihydro-1-benzofuran-5-yl}-3-{4-[(1-methylpiperidin-4-yl)carbonyl]phenyl}urea;
1-(4-{[2-(dimethylamino)ethyl](methyl)amino}phenyl)-3-{(2Z)-2-[(1-ethyl-5-methoxy-2-methyl-1H-indol-3-yl)methylene]-3-oxo-2,3-dihydro-1-benzofuran-5-yl}urea;
1-(4-{[3-(dimethylamino)propyl](methyl)amino}phenyl)-3-{(2Z)-2-[(1-ethyl-5-methoxy-2-methyl-1H-indol-3-yl)methylene]-3-oxo-2,3-dihydro-1-benzofuran-5-yl}urea;
1-{4-[3-(dimethylamino)propoxy]phenyl}-3-{(2Z)-2-[(1-ethyl-5-methoxy-2-methyl-1H-indol-3-yl)methylene]-3-oxo-2,3-dihydro-1-benzofuran-5-yl}urea;
N-[2-(dimethylamino)ethyl]-4-[({(2Z)-2-[(1-ethyl-5-methoxy-1H-indol-3-yl)methylene]-3-oxo-2,3-dihydro-1-benzofuran-5-yl}carbamoyl)amino]-N-methylbenzamide;
1-[(2Z)-2-{[5-methoxy-1-(2-piperazin-1-ylethyl)-2-(1,3,5-trimethyl-1H-pyrazol-4-yl)-1H-indol-3-yl]methylene}-3-oxo-2,3-dihydro-1-benzofuran-5-yl]-3-methylurea;
N-[3-(dimethylamino)propyl]-4-[({(2Z)-2-[(1-ethyl-5-methoxy-2-methyl-1H-indol-3-yl)methylene]-3-oxo-2,3-dihydro-1-benzofuran-5-yl}carbamoyl)amino]-N-methylbenzamide;
1-{(2Z)-2-[(5-methoxy-1,2-dimethyl-1H-indol-3-yl)methylidene]-3-oxo-2,3-dihydro-1-benzofuran-5-yl}-3-{4-[(4-methylpiperazin-1-yl)carbonyl]phenyl}urea;
4-[({(2Z)-2-[(1-ethyl-5-methoxy-2-methyl-1H-indol-3-yl)methylene]-3-oxo-2,3-dihydro-1-benzofuran-5-yl}carbamoyl)amino]-N-[2-(methylamino)ethyl]benzamide;
4-[({(2Z)-2-[(2-cyclohexyl-5-methoxy-1H-indol-3-yl)methylene]-3-oxo-2,3-dihydro-1-benzofuran-5-yl}carbamoyl)amino]-N-[2-(methylamino)ethyl]benzamide;
4-[({(2Z)-2-[(5-methoxy-1,2-dimethyl-1H-indol-3-yl)methylene]-3-oxo-2,3-dihydro-1-benzofuran-5-yl}carbamoyl)amino]-N-[2-(methylamino)ethyl]benzamide;
1-[(2Z)-2-({5-methoxy-2-methyl-1-[2-(4-methylpiperazin-1-yl)ethyl]-1H-indol-3-yl}methylidene)-7-methyl-3-oxo-2,3-dihydro-1-benzofuran-5-yl]-3-methylurea;
1-{4-[4-(dimethylamino)butyl]phenyl}-3-{(2Z)-2-[(1-ethyl-5-methoxy-2-methyl-1H-indol-3-yl)methylidene]-3-oxo-2,3-dihydro-1-benzofuran-5-yl}urea;
1-[(2Z)-2-{[1-(2-{[2-(dimethylamino)ethyl]amino}ethyl)-5-methoxy-2-(1,3,5-trimethyl-1H-pyrazol-4-yl)-1H-indol-3-yl]methylidene}-3-oxo-2,3-dihydro-1-benzofuran-5-yl]-3-methylurea;
1-[(2Z)-2-{[1-(2-{[2-(dimethylamino)ethyl](methyl)amino}ethyl)-5-methoxy-2-(1,3,5-trimethyl-1H-pyrazol-4-yl)-1H-indol-3-yl]methylidene}-3-oxo-2,3-dihydro-1-benzofuran-5-yl]-3-methylurea;
N-[3-(dimethylamino)propyl]-4-[({(2Z)-2-[(1-ethyl-5-methoxy-2-methyl-1H-indol-3-yl)methylidene]-3-oxo-2,3-dihydro-1-benzofuran-5-yl}carbamoyl)amino]benzamide;
1-{4-[2-(dimethylamino)ethoxy]phenyl}-3-{(2Z)-2-[(1-ethyl-5-methoxy-2-methyl-1H-indol-3-yl)methylidene]-3-oxo-2,3-dihydro-1-benzofuran-5-yl}urea;
1-{4-[4-(dimethylamino)butoxy]phenyl}-3-{(2Z)-2-[(1-ethyl-5-methoxy-2-methyl-1H-indol-3-yl)methylidene]-3-oxo-2,3-dihydro-1-benzofuran-5-yl}urea;
4-({[(2Z)-2-({5-methoxy-2-methyl-1-[2-(4-methylpiperazin-1-yl)ethyl]-1H-indol-3-yl}methylidene)-3-oxo-2,3-dihydro-1-benzofuran-5-yl]carbamoyl}amino)-N-[2-(methylamino)ethyl]benzamide;
1-{4-[2-(dimethylamino)ethyl]phenyl}-3-{(2Z)-2-[(1-ethyl-5-methoxy-2-methyl-1H-indol-3-yl)methylidene]-3-oxo-2,3-dihydro-1-benzofuran-5-yl}urea;
1-{4-[3-(dimethylamino)propyl]phenyl}-3-{(2Z)-2-[(1-ethyl-5-methoxy-2-methyl-1H-indol-3-yl)methylidene]-3-oxo-2,3-dihydro-1-benzofuran-5-yl}urea;
1-[(2Z)-2-({5-methoxy-1-[2-(methylamino)ethyl]-2-(1,3,5-trimethyl-1H-pyrazol-4-yl)-1H-indol-3-yl}methylidene)-3-oxo-2,3-dihydro-1-benzofuran-5-yl]-3-methylurea;
1-[(2Z)-2-({1-[2-(dimethylamino)ethyl]-5-methoxy-2-(1,3,5-trimethyl-1H-pyrazol-4-yl)-1H-indol-3-yl}methylidene)-3-oxo-2,3-dihydro-1-benzofuran-5-yl]-3-methylurea;
1-{4-[4-(dimethylamino)butoxy]phenyl}-3-[(2Z)-2-({5-methoxy-2-methyl-1-[2-(4-methylpiperazin-1-yl)ethyl]-1H-indol-3-yl}methylidene)-3-oxo-2,3-dihydro-1-benzofuran-5-yl]urea;
1-(4-{[4-(dimethylamino)butyl](methyl)amino}phenyl)-3-{(2Z)-2-[(1-ethyl-5-methoxy-2-methyl-1H-indol-3-yl)methylidene]-3-oxo-2,3-dihydro-1-benzofuran-5-yl}urea;
N-{4-[({(2Z)-2-[(1-ethyl-5-methoxy-2-methyl-1H-indol-3-yl)methylidene]-3-oxo-2,3-dihydro-1-benzofuran-5-yl}carbamoyl)amino]phenyl}-N3,N3-dimethyl-b-alaninamide;
1-{(2Z)-2-[(1-ethyl-5-methoxy-2-methyl-1H-indol-3-yl)methylidene]-3-oxo-2,3-dihydro-1-benzofuran-5-yl}-3-{4-[(4-methylpiperazin-1-yl)sulfonyl]phenyl}urea;
1-(4-{[2-(dimethylamino)ethyl]amino}phenyl)-3-{(2Z)-2-[(1-ethyl-5-methoxy-2-methyl-1H-indol-3-yl)methylidene]-3-oxo-2,3-dihydro-1-benzofuran-5-yl}urea;
N-[2-(dimethylamino)ethyl]-4-[({(2Z)-2-[(1-ethyl-5-methoxy-2-methyl-1H-indol-3-yl)methylidene]-3-oxo-2,3-dihydro-1-benzofuran-5-yl}carbamoyl)amino]-N-methylbenzenesulfonamide;
1-[(2Z)-2-{[5-(2-methoxyethoxy)-2-(1,3,5-trimethyl-1H-pyrazol-4-yl)-1H-indol-3-yl]methylidene}-3-oxo-2,3-dihydro-1-benzofuran-5-yl]-3-methylurea;
1-[4-(dimethylamino)phenyl]-3-{(2Z)-2-[(1-ethyl-5-methoxy-2-methyl-1H-indol-3-yl)methylidene]-3-oxo-2,3-dihydro-1-benzofuran-5-yl}urea;
N-[2-(dimethylamino)ethyl]-4-({[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]carbamoyl}amino)-N-methylbenzamide;
1-{(2Z)-2-[(1-ethyl-5-methoxy-2-methyl-1H-indol-3-yl)methylidene]-3-oxo-2,3-dihydro-1-benzofuran-5-yl}-3-{4-[3-(methylamino)propoxy]phenyl}urea;
1-(4-{[2-(dimethylamino)ethyl]amino}phenyl)-3-[(2Z)-2-({5-methoxy-2-methyl-1-[2-(4-methylpiperazin-1-yl)ethyl]-1H-indol-3-yl}methylidene)-3-oxo-2,3-dihydro-1-benzofuran-5-yl]urea;
N-[4-({[(2Z)-2-({5-methoxy-2-methyl-1-[2-(4-methylpiperazin-1-yl)ethyl]-1H-indol-3-yl}methylidene)-3-oxo-2,3-dihydro-1-benzofuran-5-yl]carbamoyl}amino)phenyl]-N3,N3-dimethyl-b-alaninamide;
1-{(2Z)-2-[(1-ethyl-5-methoxy-2-methyl-1H-indol-3-yl)methylidene]-3-oxo-2,3-dihydro-1-benzofuran-5-yl}-3-[4-(morpholin-4-ylcarbonyl)phenyl]urea;
1-[(2Z)-2-({5-methoxy-2-methyl-1-[2-(4-methylpiperazin-1-yl)ethyl]-1H-indol-3-yl}methylidene)-3-oxo-2,3-dihydro-1-benzofuran-5-yl]-3-[4-(morpholin-4-ylcarbonyl)phenyl]urea;
4-[({(2Z)-2-[(2-cyclohexyl-5-methoxy-1H-indol-3-yl)methylidene]-3-oxo-2,3-dihydro-1-benzofuran-5-yl}carbamoyl)amino]-N-[2-(dimethylamino)ethyl]-N-methylbenzamide;
1-[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]-3-{4-[(4-methylpiperazin-1-yl)carbonyl]phenyl}urea;
1-[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]-3-[4-(morpholin-4-ylcarbonyl)phenyl]urea;
N-[4-({[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]carbamoyl}amino)phenyl]-N3,N3-dimethyl-b-alaninamide;
1-{(2Z)-2-[(2-bromo-5-methoxy-1H-indol-3-yl)methylidene]-3-oxo-2,3-dihydro-1-benzofuran-5-yl}-3-methylurea;
1-[(2Z)-2-{[7-fluoro-5-methoxy-2-(1,3,5-trimethyl-1H-pyrazol-4-yl)-1H-indol-3-yl]methylidene}-3-oxo-2,3-dihydro-1-benzofuran-5-yl]-3-methylurea;
N-{4-[({(2Z)-2-[(1-ethyl-5-methoxy-2-methyl-1H-indol-3-yl)methylidene]-3-oxo-2,3-dihydro-1-benzofuran-5-yl}carbamoyl)amino]phenyl}-N,N3,N3-trimethyl-b-alaninamide;
N-[4-({[(2Z)-2-({5-methoxy-2-methyl-1-[2-(4-methylpiperazin-1-yl)ethyl]-1H-indol-3-yl}methylidene)-3-oxo-2,3-dihydro-1-benzofuran-5-yl]carbamoyl}amino)phenyl]-N,N3,N3-trimethyl-b-alaninamide;
N-(4-{[(2-{[5-methoxy-2-(1,3,5-trimethyl-1H-pyrazol-4-yl)-1H-indol-3-yl]methylidene}-3-oxo-2,3-dihydro-1-benzofuran-5-yl)carbamoyl]amino}phenyl)-N,N3,N3-trimethyl-b-alaninamide;
1-(4-{[3-(dimethylamino)pyrrolidin-1-yl]carbonyl}phenyl)-3-[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]urea;
4-({[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]carbamoyl}amino)-N-methyl-N-(1-methylpyrrolidin-3-yl)benzamide;
1-{4-[(4-ethylpiperazin-1-yl)carbonyl]phenyl}-3-[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]urea;
1-[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]-3-(4-{[4-(1-methylethyl)piperazin-1-yl]carbonyl}phenyl)urea;
4-({[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]carbamoyl}amino)-N-methyl-N-(2-pyrrolidin-1-ylethyl)benzamide;
1-(4-{[4-(dimethylamino)piperidin-1-yl]carbonyl}phenyl)-3-[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]urea;
1-{4-[(3,4-dimethylpiperazin-1-yl)carbonyl]phenyl}-3-[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]urea;
4-({[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]carbamoyl}amino)-N,N-dimethylbenzamide;
1-{(2Z)-2-[(2-{1-[2-(dimethylamino)ethyl]-3,5-dimethyl-1H-pyrazol-4-yl}-5-methoxy-1H-indol-3-yl)methylidene]-3-oxo-2,3-dihydro-1-benzofuran-5-yl}-3-methylurea;
1-[(2Z)-2-({5-methoxy-2-[1-(2-methylpropyl)-1H-pyrazol-4-yl]-1H-indol-3-yl}methylidene)-3-oxo-2,3-dihydro-1-benzofuran-5-yl]-3-methylurea;
N-[2-(dimethylamino)ethyl]-3-({[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]carbamoyl}amino)-N-methylbenzamide;
N-[3-(dimethylamino)propyl]-3-({[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]carbamoyl}amino)-N-methylbenzamide;
1-[(2Z)-2-({5-methoxy-2-[1-(2-methoxyethyl)-3,5-dimethyl-1H-pyrazol-4-yl]-1H-indol-3-yl}methylidene)-3-oxo-2,3-dihydro-1-benzofuran-5-yl]-3-methylurea;
N-[2-(dimethylamino)ethyl]-4-({[(2Z)-2-{[7-fluoro-5-methoxy-2-(1,3,5-trimethyl-1H-pyrazol-4-yl)-1H-indol-3-yl]methylidene}-3-oxo-2,3-dihydro-1-benzofuran-5-yl]carbamoyl}amino)-N-methylbenzamide;
N-[2-(dimethylamino)ethyl]-4-({[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]carbamoyl}amino)benzamide;
1-[(2Z)-2-{[6-fluoro-5,7-dimethoxy-2-(1,3,5-trimethyl-1H-pyrazol-4-yl)-1H-indol-3-yl]methylidene}-3-oxo-2,3-dihydro-1-benzofuran-5-yl]-3-methylurea;
1-[(2Z)-2-{[6,7-difluoro-5-methoxy-2-(1,3,5-trimethyl-1H-pyrazol-4-yl)-1H-indol-3-yl]methylidene}-3-oxo-2,3-dihydro-1-benzofuran-5-yl]-3-methylurea;
1-[(2Z)-2-{[2-(3,5-dimethyl-1H-pyrazol-4-yl)-7-fluoro-5-methoxy-1H-indol-3-yl]methylidene}-3-oxo-2,3-dihydro-1-benzofuran-5-yl]-3-methylurea;
1-(2-aminoethyl)-3-[(2Z)-2-{[7-fluoro-5-methoxy-2-(1,3,5-trimethyl-1H-pyrazol-4-yl)-1H-indol-3-yl]methylidene}-3-oxo-2,3-dihydro-1-benzofuran-5-yl]urea;
1-[2-(dimethylamino)ethyl]-3-[(2Z)-2-{[7-fluoro-5-methoxy-2-(1,3,5-trimethyl-1H-pyrazol-4-yl)-1H-indol-3-yl]methylidene}-3-oxo-2,3-dihydro-1-benzofuran-5-yl]urea;
1-[(2Z)-2-({7-fluoro-5-methoxy-2-[1-(2-methylpropyl)-1H-pyrazol-4-yl]-1H-indol-3-yl}methylidene)-3-oxo-2,3-dihydro-1-benzofuran-5-yl]-3-methylurea;
1-[(2Z)-2-{[7-fluoro-5-methoxy-2-(1-methyl-1H-pyrazol-4-yl)-1H-indol-3-yl]methylidene}-3-oxo-2,3-dihydro-1-benzofuran-5-yl]-3-methylurea;
1-{4-[(dimethylamino)methyl]phenyl}-3-{(2Z)-2-[(1-ethyl-5-methoxy-2-methyl-1H-indol-3-yl)methylidene]-3-oxo-2,3-dihydro-1-benzofuran-5-yl}urea;
1-{4-[(1,1-dioxidothiomorpholin-4-yl)carbonyl]phenyl}-3-[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]urea;
1-[(2Z)-2-{[7-fluoro-5-methoxy-2-(1,3,5-trimethyl-1H-pyrazol-4-yl)-1H-indol-3-yl]methylidene}-3-oxo-2,3-dihydro-1-benzofuran-5-yl]-3-(4-{[4-(2-hydroxyethyl)piperazin-1-yl]carbonyl}phenyl)urea;
1-[(2Z)-2-{[2-(1,3-dimethyl-1H-pyrazol-4-yl)-5-methoxy-1H-indol-3-yl]methylidene}-3-oxo-2,3-dihydro-1-benzofuran-5-yl]-3-methylurea;
1-[(2Z)-2-{[2-(1,5-dimethyl-1H-pyrazol-4-yl)-5-methoxy-1H-indol-3-yl]methylidene}-3-oxo-2,3-dihydro-1-benzofuran-5-yl]-3-methylurea;
1-[(2Z)-2-({5-methoxy-2-[1-methyl-4-(trifluoromethyl)-1H-pyrazol-3-yl]-1H-indol-3-yl}methylidene)-3-oxo-2,3-dihydro-1-benzofuran-5-yl]-3-methylurea;
1-[(2Z)-2-{[5-methoxy-7-(trifluoromethyl)-2-(1,3,5-trimethyl-1H-pyrazol-4-yl)-1H-indol-3-yl]methylidene}-3-oxo-2,3-dihydro-1-benzofuran-5-yl]-3-methylurea;
1-[(2Z)-2-{[5-methoxy-7-methyl-2-(1,3,5-trimethyl-1H-pyrazol-4-yl)-1H-indol-3-yl]methylidene}-3-oxo-2,3-dihydro-1-benzofuran-5-yl]-3-methylurea;
1-[(2Z)-2-{[7-fluoro-5-methoxy-2-(1,3,5-trimethyl-1H-pyrazol-4-yl)-1H-indol-3-yl]methylidene}-3-oxo-2,3-dihydro-1-benzofuran-5-yl]-3-(2-pyrrolidin-1-ylethyl)urea;
1-[(2Z)-2-{[7-fluoro-5-methoxy-2-(1,3,5-trimethyl-1H-pyrazol-4-yl)-1H-indol-3-yl]methylidene}-3-oxo-2,3-dihydro-1-benzofuran-5-yl]-3-[2-(methylamino)ethyl]urea;
1-(4-{[4-(dimethylamino)piperidin-1-yl]carbonyl}phenyl)-3-[(2Z)-2-{[7-fluoro-5-methoxy-2-(1,3,5-trimethyl-1H-pyrazol-4-yl)-1H-indol-3-yl]methylidene}-3-oxo-2,3-dihydro-1-benzofuran-5-yl]urea;
1-{4-[(3,4-dimethylpiperazin-1-yl)carbonyl]phenyl}-3-[(2Z)-2-{[7-fluoro-5-methoxy-2-(1,3,5-trimethyl-1H-pyrazol-4-yl)-1H-indol-3-yl]methylidene}-3-oxo-2,3-dihydro-1-benzofuran-5-yl]urea;
1-[(2Z)-2-{[7-fluoro-5-methoxy-2-(1,3,5-trimethyl-1H-pyrazol-4-yl)-1H-indol-3-yl]methylidene}-3-oxo-2,3-dihydro-1-benzofuran-5-yl]-3-[4-(morpholin-4-ylcarbonyl)phenyl]urea;
1-(4-{[3-(dimethylamino)pyrrolidin-1-yl]carbonyl}phenyl)-3-[(2Z)-2-{[7-fluoro-5-methoxy-2-(1,3,5-trimethyl-1H-pyrazol-4-yl)-1H-indol-3-yl]methylidene}-3-oxo-2,3-dihydro-1-benzofuran-5-yl]urea; and
N-[2-(dimethylamino)ethyl]-4-({[(2Z)-2-{[7-fluoro-5-methoxy-2-(1,3,5-trimethyl-1H-pyrazol-4-yl)-1H-indol-3-yl]methylidene}-3-oxo-2,3-dihydro-1-benzofuran-5-yl]carbamoyl}amino)benzamide;

In other aspects, the invention provides pharmaceutical compositions comprising compounds or pharmaceutically acceptable salts of the compounds of the present Formula I and a pharmaceutically acceptable carrier.

In other aspects, the invention provides that the pharmaceutically acceptable carrier suitable for oral administration and the composition comprises an oral dosage form.

In other aspects, the invention provides a composition comprising a compound of Formula I; a second compound selected from the group consisting of a topoisomerase I inhibitor, a MEK1/2 inhibitor, a HSP90 inhibitor, procarbazine, dacarbazine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epirubicin, 5-fluorouracil, docetaxel, paclitaxel, leucovorin, levamisole, irinotecan, estramustine, etoposide, nitrogen mustards, BCNU, carmustine, lomustine, vinblastine, vincristine, vinorelbine, cisplatin, carboplatin, oxaliplatin, imatinib mesylate, Avastin (bevacizumab), hexamethylmelamine, topotecan, tyrosine kinase inhibitors, tyrphostins, herbimycin A, genistein, erbstatin, hydroxyzine, glatiramer acetate, interferon beta-1a, interferon beta-1b, natalizumab, and lavendustin A; and a pharmaceutically acceptable carrier.

In other aspects, the second compound is Avastin.

In other aspects, the invention provides a method of treating a PI3K-related disorder, comprising administering to a mammal in need thereof a compound of Formula I in an amount effective to treat a PI3K-related disorder.

In other aspects, the PI3K-related disorder is selected from restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, and cancer.

In other aspects, the PI3K-related disorder is cancer.

In other aspects, the cancer is selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer.

In other aspects, the invention provides a method of treating an mTOR-related disorder, comprising administering to a mammal in need thereof a compound of Formula I in an amount effective to treat an mTOR-related disorder.

In other aspects, the mTOR-related disorder is selected from restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, and cancer.

In other aspects, the mTOR-related disorder is cancer.

In other aspects, the cancer is selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer.

In other aspects, the invention provides a method of treating advanced renal cell carcinoma, comprising administering to a mammal in need thereof a compound of Formula I in an amount effective to treat advanced renal cell carcinoma.

In other aspects, the invention provides a method of treating acute lymphoblastic leukemia, comprising administering to a mammal in need thereof a compound of Formula I in an amount effective to treat acute lymphoblastic leukemia.

In other aspects, the invention provides a method of treating acute malignant melanoma, comprising administering to a mammal in need thereof a compound of Formula I in an amount effective to treat malignant melanoma.

In other aspects, the invention provides a method of treating soft-tissue or bone sarcoma, comprising administering to a mammal in need thereof a compound of Formula I in an amount effective to treat soft-tissue or bone sarcoma.

In other aspects, the invention provides a method of treating a cancer selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer comprising administering to a mammal in need thereof a composition comprising a compound of Formula I; a second compound selected from the group consisting of a topoisomerase I inhibitor, a MEK1/2 inhibitor, a HSP90 inhibitor, procarbazine, dacarbazine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epirubicin, 5-fluorouracil, docetaxel, paclitaxel, leucovorin, levamisole, irinotecan, estramustine, etoposide, nitrogen mustards, BCNU, carmustine, lomustine, vinblastine, vincristine, vinorelbine, cisplatin, carboplatin, oxaliplatin, imatinib mesylate, Avastin (bevacizumab), hexamethylmelamine, topotecan, tyrosine kinase inhibitors, tyrphostins, herbimycin A, genistein, erbstatin, hydroxyzine, glatiramer acetate, interferon beta-1a, interferon beta-1b, natalizumab, and lavendustin A; and a pharmaceutically acceptable carrier. in an amount effective to treat the cancer.

In other aspects, the invention provides a method of inhibiting mTOR in a subject, comprising administering to a subject in need thereof a compound of Formula I in an amount effective to inhibit mTOR.

In other aspects, the invention provides a method of inhibiting PI3K in a subject, comprising administering to a subject in need thereof a compound of Formula I in an amount effective to inhibit PI3K.

In other aspects, the invention provides a method of inhibiting mTOR and PI3K together in a subject, comprising administering to a subject in need thereof a compound of Formula I in an amount effective to inhibit mTOR and PI3K.

In other aspects, the invention provides a method of synthesizing a compound of Formula I′, comprising:

a) condensing a compound of the formula CXI with a compound of formula CXII:

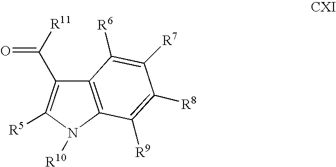

under acidic conditions, and A and R¹-R¹¹ are as defined above in formula I

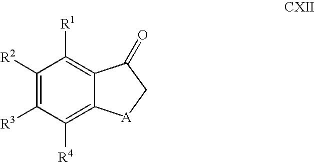

thereby producing a compound of formula I′:

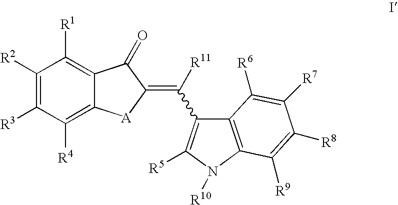

b) optionally reducing the compound of formula I′ and thereby producing a compound of formula I″:

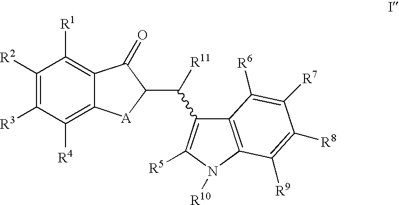

or a pharmaceutically acceptable salt thereof.

In other aspects, the invention provides the method further comprising:

a) acylation with R¹¹C(O)X, wherein X is halogen, or Vilsmeier-Haack formylation, of a compound of formula CIX:

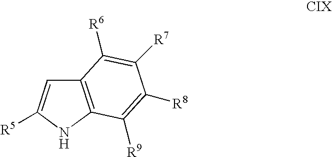

thereby producing a compound of formula CX:

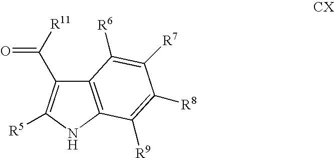

b) optionally alkylating the compound of formula CX with R¹⁰Cl, thereby producing a compound of Formula CXI.

Representative “pharmaceutically acceptable salts” include but are not limited to, e.g., water-soluble and water-insoluble salts, such as the acetate, aluminum, amsonate (4,4-diaminostilbene-2,2-disulfonate), benzathine (N,N′-dibenzylethylenediamine), benzenesulfonate, benzoate, bicarbonate, bismuth, bisulfate, bitartrate, borate, bromide, butyrate, calcium, calcium edetate, camsylate (camphorsulfonate), carbonate, chloride, choline, citrate, clavulariate, diethanolamine, dihydrochloride, diphosphate, edetate, edisylate (camphorsulfonate), esylate (ethanesulfonate), ethylenediamine, fumarate, gluceptate (glucoheptonate), gluconate, glucuronate, glutamate, hexafluorophosphate, hexylresorcinate, hydrabamine (N,N′-bis(dehydroabietyl)ethylenediamine), hydrobromide, hydrochloride, hydroxynaphthoate, 1-hydroxy-2-naphthoate, 3-hydroxy-2-naphthoate, iodide, isothionate (2-hydroxyethanesulfonate), lactate, lactobionate, laurate, lauryl sulfate, lithium, magnesium, malate, maleate, mandelate, meglumine (1-deoxy-1-(methylamino)-D-glucitol), mesylate, methyl bromide, methylnitrate, methylsulfate, mucate, napsylate, nitrate, N-methylglucamine ammonium salt, oleate, oxalate, palmitate, pamoate (4,4′-methylenebis-3-hydroxy-2-naphthoate, or embonate), pantothenate, phosphate, picrate, polygalacturonate, potassium, propionate, p-toluenesulfonate, salicylate, sodium, stearate, subacetate, succinate, sulfate, sulfosaliculate, suramate, tannate, tartrate, teoclate (8-chloro-3,7-dihydro-1,3-dimethyl-1H-purine-2,6-dione), triethiodide, tromethamine (2-amino-2-(hydroxymethyl)-1,3-propanediol), valerate, and zinc salts.

Some compounds within the present invention possess one or more chiral centers, and the present invention includes each separate enantiomer of such compounds as well as mixtures of the enantiomers. Where multiple chiral centers exist in compounds of the present invention, the invention includes each combination as well as mixtures thereof. All chiral, diastereomeric, and racemic forms of a structure are intended, unless the specific stereochemistry or isomeric form is specifically indicated. It is well known in the art how to prepare optically active forms, such as by resolution of racemic forms or by synthesis from optically active starting materials.

In some embodiments, the compounds within the present invention possess double bonds connecting the pyrrolo-pyridine moiety to the benzofuran or benzothiophene nucleolus. These double bonds can exist as geometric isomers, and the invention includes both E and Z isomers of such double bonds. All such stable isomers are contemplated in the present invention.

An “effective amount” when used in connection a compound of the present invention of this invention is an amount effective for inhibiting mTOR or PI3K in a subject.

### DEFINITIONS

The following definitions are used in connection with the compounds of the present invention unless the context indicates otherwise. In general, the number of carbon atoms present in a given group is designated “Cₓ-C_{y}” where x and y are the lower and upper limits, respectively. For example, a group designated as “C₁-C₆” contains from 1 to 6 carbon atoms. The carbon number as used in the definitions herein refers to carbon backbone and carbon branching, but does not include carbon atoms of the substituents, such as alkoxy substitutions and the like. Unless indicated otherwise, the nomenclature of substituents that are not explicitly defined herein are arrived at by naming from left to right the terminal portion of the functionality followed by the adjacent functionality toward the point of attachment. For example, the substituent “arylalkyloxycabonyl” refers to the group (C₆-C₁₄aryl)-(C₁-C₆alkyl)-O—C(O)—. It is understood that the definitions below are not intended to include impermissible substitution patterns (e.g., methyl substituted with 5 fluoro groups). Such impermissible substitution patterns are well known to the skilled artisan.

Acyl” refers to a group having a straight, branched, or cyclic configuration or a combination thereof, attached to the parent structure through a carbonyl functionality. Such groups may be saturated or unsaturated, aliphatic or aromatic, and carbocyclic or heterocyclic. Examples of a C₁-C₈acyl group include acetyl-, benzoyl-, nicotinoyl, propionyl-, isobutyryl-, oxalyl-, and the like. Lower-acyl refers to acyl groups containing one to four carbons. An acyl group can be unsubstituted or substituted with one or more of the following groups: halogen, —NH₂, C₁-C₆aminoalkyl-, di(C₁-C₆alkyl)amino-, —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, or C₃-C₈cycloalkyl.

“Alkenyl” refer to a straight or branched chain unsaturated hydrocarbon containing at least one double bond. Examples of a C₂-C₁₀alkenyl group include, but are not limited to, ethylene, propylene, 1-butylene, 2-butylene, isobutylene, sec-butylene, 1-pentene, 2-pentene, isopentene, 1-hexene, 2-hexene, 3-hexene, isohexene, 1-heptene, 2-heptene, 3-heptene, 1-octene, 2-octene, 3-octene, 4-octene, 1-nonene, 2-nonene, 3-nonene, 4-nonene, 1-decene, 2-decene, 3-decene, 4-decene and 5-decene. A C₂-C₁₀alkenyl group can be unsubstituted or substituted with one or more of the following groups: halogen, —NH₂, (C₁-C₆alkyl)NH—, di(C₁-C₆alkyl)amino-, —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆alkoxy, C₁-C₆alkyl, —C(O)OH, (C₁-C₆alkoxy)carbonyl, —C(O)(C₁-C₆alkyl), C₆-C₁₄aryl C₁-C₉heteroaryl, and C₃-C₈cycloalkyl.

“Alkoxy” refers to the group R—O— where R is an alkyl group, as defined below. Exemplary C₁-C₆alkoxy groups include but are not limited to methoxy, ethoxy, n-propoxy, 1-propoxy, n-butoxy and t-butoxy. An alkoxy group can be unsubstituted or substituted with one or more of the following groups: halogen, hydroxyl, C₁-C₆alkoxy, —NH₂, (C₁-C₆alkyl)NH—, di(C₁-C₆alkyl)amino-, —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, C₁-C₆alkoxy, —C(O)OH, (C₁-C₆alkoxy)carbonyl, —C(O)(C₁-C₆alkyl), C₆-C₁₄aryl, C₁-C₉heteroaryl, C₃-C₈cycloalkyl, C₁-C₆haloalkyl-, C₁-C₆aminoalkyl-, —OC(O)(C₁-C₆alkyl), C₁-C₆carboxyamidoalkyl-, or —NO₂;

“(Alkoxy)carbonyl” refers to the group alkyl-O—C(O)—. Exemplary (C₁-C₆alkoxy)carbonyl groups include but are not limited to methoxy, ethoxy, n-propoxy, 1-propoxy, n-butoxy and t-butoxy. An (alkoxy)carbonyl group can be unsubstituted or substituted with one or more of the following groups: halogen, hydroxyl, —NH₂, (C₁-C₆alkyl)NH—, di(C₁-C₆alkyl)amino-, —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, C₁-C₆alkoxy, —C(O)OH, (C₁-C₆alkoxy)carbonyl, —C(O)(C₁-C₆alkyl), C₆-C₁₄aryl, C₁-C₉heteroaryl, C₃-C₈cycloalkyl, C₁-C₆haloalkyl-, C₁-C₆aminoalkyl-, —OC(O)(C₁-C₆alkyl), C₁-C₆carboxyamidoalkyl-, or —NO₂.

“Alkyl” refers to a hydrocarbon chain that may be a straight chain or branched chain, containing the indicated number of carbon atoms, for example, a C₁-C₁₀alkyl group may have from 1 to 10 (inclusive) carbon atoms in it. In the absence of any numerical designation, “alkyl” is a chain (straight or branched) having 1 to 6 (inclusive) carbon atoms in it. Examples of C₁-C₆ alkyl groups include, but are not limited to, methyl, ethyl, propyl, butyl, pentyl, hexyl, isopropyl, isobutyl, sec-butyl, tert-butyl, isopentyl, neopentyl, and isohexyl. An alkyl group can be unsubstituted or substituted with one or more of the following groups: halogen, —NH₂, (C₁-C₆alkyl)NH—, di(C₁-C₆alkyl)amino-, —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆alkoxy, C₁-C₆alkyl, —C(O)OH, (C₁-C₆alkoxy)carbonyl, —C(O)(C₁-C₆alkyl), C₆-C₁₄aryl, C₁-C₉heteroaryl, C₃-C₈cycloalkyl, C₁-C₆haloalkyl-, C₁-C₆aminoalkyl-, —OC(O)(C₁-C₆alkyl), C₁-C₆carboxyamidoalkyl-, or —NO₂.

“(Alkyl)amido-” refers to a —C(O)NH— group in which the nitrogen atom of said group is attached to a alkyl group, as defined above. Representative examples of a (C₁-C₆alkyl)amido group include, but are not limited to, —C(O)NHCH₃, —C(O)NHCH₂CH₃, —C(O)NHCH₂CH₂CH₃, —C(O)NHCH₂CH₂CH₂CH₃, —C(O)NHCH₂CH₂CH₂CH₂CH₃, —C(O)NHCH(CH₃)₂, —C(O)NHCH₂CH(CH₃)₂, —C(O)NHCH(CH₃)CH₂CH₃, —C(O)NH—C(CH₃)₃ and —C(O)NHCH₂C(CH₃)₃.

“(Alkyl)amino-” refers to an —NH group, the nitrogen atom of said group being attached to a alkyl group, as defined above. Representative examples of an (C₁-C₆alkyl)amino group include, but are not limited to —NHCH₃, —NHCH₂CH₃, —NHCH₂CH₂CH₃, —NHCH₂CH₂CH₂CH₃, —NHCH(CH₃)₂, —NHCH₂CH(CH₃)₂, —NHCH(CH₃)CH₂CH₃, and —NH—C(CH₃)₃. An (alkyl)amino group can be unsubstituted or substituted with one or more of the following groups: halogen, —NH₂, (C₁-C₆alkyl)NH—, di(C₁-C₆alkyl)amino-, —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆alkoxy, C₁-C₆alkyl, —C(O)OH, (C₁-C₆alkoxy)carbonyl, —C(O)(C₁-C₆alkyl), C₆-C₁₄aryl, C₁-C₉heteroaryl, C₃-C₈cycloalkyl, C₁-C₆haloalkyl-, C₁-C₆aminoalkyl-, —OC(O)(C₁-C₆alkyl), C₁-C₆carboxyamidoalkyl-, or —NO₂.

“Alkylcarboxy” refers to an alkyl group, defined above, attached to the parent structure through the oxygen atom of a carboxyl (C(O)—O—) functionality. Examples of (C₁-C₆alkyl)carboxyl include acetoxy, ethylcarboxy, propylcarboxy, and isopentylcarboxy.

“(Alkyl)carboxyamido-” refers to a —NHC(O)— group in which the carbonyl carbon atom of said group is attached to a alkyl group, as defined above. Representative examples of a (C₁-C₆alkyl)carboxyamido group include, but are not limited to, —NHC(O)CH₃, —NHC(O)CH₂CH₃, —NHC(O)CH₂CH₂CH₃, —NHC(O)CH₂CH₂CH₂CH₃, —NHC(O)CH₂CH₂CH₂CH₂CH₃, —NHC(O)CH(CH₃)₂, —NHC(O)CH₂CH(CH₃)₂, —NHC(O)CH(CH₃)CH₂CH₃, —NHC(O)—C(CH₃)₃ and —NHC(O)CH₂C(CH₃)₃.

“Alkylene”, “alkenylene”, and “alkynylene” refers to alkyl, alkenyl, and alkynyl groups, as defined above, having two points of attachment within a chemical structure. Examples of C₁-C₆alkylene include ethylene (—CH₂CH₂—), propylene (—CH₂CH₂CH₂—), and dimethylpropylene (—CH₂C(CH₃)₂CH₂—). Likewise, examples of C₂-C₆alkenylene include ethenylene (—CH═CH— and propenylene (—CH═CH—CH₂—). Examples of C₂-C₆alkynylene include ethynylene (—C≡C—) and propynylene (—C≡C—CH₂—).

“Alkylthio” refers to the group R—S— where R is an alkyl group, as defined above, attached to the parent structure through a sulfur atom. Examples of C₁-C₆alkylthio include methylthio, ethylthio, n-propylthio, i-propylthio, n-butylthio, i-butylthio, s-butylthio, t-butylthio, n-pentylthio, and n-hexylthio.

“Alkynyl” refers to a straight or branched chain unsaturated hydrocarbon containing at least one triple bond. Examples of a C₂-C₁₀alkynyl group include, but are not limited to, acetylene, propyne, 1-butyne, 2-butyne, isobutyne, sec-butyne, 1-pentyne, 2-pentyne, isopentyne, 1-hexyne, 2-hexyne, 3-hexyne, isohexyne, 1-heptyne, 2-heptyne, 3-heptyne, 1-octyne, 2-octyne, 3-octyne, 4-octyne, 1-nonyne, 2-nonyne, 3-nonyne, 4-nonyne, 1-decyne, 2-decyne, 3-decyne, 4-decyne and 5-decyne. An alkynyl group can be unsubstituted or substituted with one or more of the following groups: halogen, —NH₂, (C₁-C₆alkyl)NH—, di(C₁-C₆alkyl)amino-, —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆alkoxy, C₁-C₆alkyl, —C(O)OH, (C₁-C₆alkoxy)carbonyl, —C(O)(C₁-C₆alkyl), C₆-C₁₄aryl C₁-C₉heteroaryl, and C₃-C₈cycloalkyl.

“Amido(aryl)-” refers to an aryl group, as defined below, wherein one of the aryl group's hydrogen atoms has been replaced with one or more —C(O)NH₂ groups. Representative examples of an amido(C₆-C₁₄aryl)-group include 2-C(O)NH₂-phenyl, 3-C(O)NH₂-phenyl, 4-C(O)NH₂-phenyl, 1-C(O)NH₂-naphthyl, and 2-C(O)NH₂-naphthyl.

“Aminoalkyl-” refers to an alkyl group, as defined above, wherein one or more of the alkyl group's hydrogen atoms has been replaced with —NH₂. Representative examples of an C₁-C₆aminoalkyl-group include, but are not limited to —CH₂NH₂, —CH₂CH₂NH₂, —CH₂CH₂CH₂NH₂, —CH₂CH₂CH₂CH₂NH₂, —CH₂CH(NH₂)CH₃, —CH₂CH(NH₂)CH₂CH₃, —CH(NH₂)CH₂CH₃, —C(CH₃)₂(CH₂NH₂), —CH₂CH₂CH₂CH₂CH₂NH₂, and —CH₂CH₂CH(NH₂)CH₂CH₃. An aminoalkyl-group can be unsubstituted or substituted with one or two of the following groups: C₁-C₆alkoxy, C₆-C₁₄aryl, C₁-C₉heteroaryl, C₃-C₈cycloalkyl, and C₁-C₆alkyl.

Aryl refers to an aromatic hydrocarbon group. Examples of an C₆-C₁₄aryl group include, but are not limited to, phenyl, 1-naphthyl, 2-naphthyl, 3-biphen-1-yl, anthryl, tetrahydronaphthyl, fluorenyl, indanyl, biphenylenyl, and acenaphthenyl. An aryl group can be unsubstituted or substituted with one or more of the following groups: C₁-C₆alkyl, halo, haloalkyl-, hydroxyl, hydroxyl(C₁-C₆alkyl)-, —NH₂, aminoalkyl-, di(C₁-C₆alkyl)amino-, —COOH, —C(O)O—(C₁-C₆alkyl), —OC(O)(C₁-C₆alkyl), N-alkylamido-, —C(O)NH₂, (C₁-C₆alkyl)amido-, or —NO₂.

“(Aryl)alkyl” refers to an alkyl group, as defined above, wherein one or more of the alkyl group's hydrogen atoms has been replaced with an aryl group as defined above. (C₆-C₁₄Aryl)alkyl moieties include benzyl, 1-phenylethyl, 2-phenylethyl, 3-phenylpropyl, 2-phenylpropyl, 1-naphthylmethyl, 2-naphthylmethyl, and the like. An (aryl)alkyl group can be unsubstituted or substituted with one or more of the following groups: halogen, —NH₂, hydroxyl, (C₁-C₆alkyl)NH—, di(C₁-C₆alkyl)amino-, —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆alkoxy, C₁-C₆alkyl, —C(O)OH, (C₁-C₆alkoxy)carbonyl, —C(O)(C₁-C₆alkyl), C₆-C₁₄aryl, C₁-C₉heteroaryl, C₃-C₈cycloalkyl, C₁-C₆haloalkyl-, C₁-C₆aminoalkyl-, —OC(O)(C₁-C₆alkyl), C₁-C₆carboxyamidoalkyl-, or —NO₂.

“(Aryl)amino” refers to a radical of formula (aryl)-NH—, wherein aryl is as defined above. Examples of (C₆-C₁₄aryl)amino radicals include, but are not limited to, phenylamino (anilido), 1-naphthlamino, 2-naphthlamino, and the like. An (C₆-C₁₄aryl)amino group can be unsubstituted or substituted with one or more of the following groups: halogen, —NH₂, (C₁-C₆alkyl)NH—, di(C₁-C₆alkyl)amino-, —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆alkoxy, C₁-C₆alkyl, —C(O)OH, (C₁-C₆alkoxy)carbonyl, —C(O)(C₁-C₆alkyl), C₆-C₁₄aryl C₁-C₉heteroaryl, or C₃-C₈cycloalkyl.

“(Aryl)oxy” refers to the group Ar—O— where Ar is an aryl group, as defined above. Exemplary (C₆-C₁₄aryl)oxy groups include but are not limited to phenyloxy, α-naphthyloxy, and β-naphthyloxy. An (aryl)oxy group can be unsubstituted or substituted with one or more of the following groups: C₁-C₆alkyl, halo, C₁-C₆haloalkyl-, hydroxyl, C₁-C₆hydroxylalkyl-, —NH₂, C₁-C₆aminoalkyl-, -dialkylamino-, —COOH, —C(O)O—(C₁-C₆alkyl), —OC(O)(C₁-C₆alkyl), N-alkylamido-, —C(O)NH₂, (C₁-C₆alkyl)amido-, or —NO₂.

“Cycloalkyl” refers to a monocyclic, non-aromatic, saturated hydrocarbon ring. Representative examples of a C₃-C₈cycloalkyl include, but are not limited to, cyclopropyl, cyclobutyl, cyclopentyl, cyclohexyl, cycloheptyl, and cyclooctyl. A cycloalkyl can be unsubstituted or independently substituted with one or more of the following groups: halogen, —NH₂, (C₁-C₆alkyl)NH—, di(C₁-C₆alkyl)amino-, —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆alkoxy, C₁-C₆alkyl, —C(O)OH, (C₁-C₆alkoxy)carbonyl, —C(O)(C₁-C₆alkyl), C₆-C₁₄aryl, C₁-C₉heteroaryl, or C₃-C₈cycloalkyl, C₁-C₆haloalkyl-, C₁-C₆aminoalkyl-, —OC(O)(C₁-C₆alkyl), C₁-C₆carboxyamidoalkyl-, or —NO₂. Additionally, each of any two hydrogen atoms on the same carbon atom of the carbocyclic ring can be replaced by an oxygen atom to form an oxo (═O) substituent or the two hydrogen atoms can be replaced by an alkylenedioxy group so that the alkylenedioxy group, when taken together with the carbon atom to which it is attached, form a 5- to 7-membered heterocycle containing two oxygen atoms.

“Bicyclic cycloalkyl” refers to a bicyclic, non-aromatic, saturated hydrocarbon ring system. Representative examples of a C₆-C₁₀bicyclic cycloalkyl include, but are not limited to, cis-1-decalinyl, trans 2-decalinyl, cis-4-perhydroindanyl, and trans-7-perhydroindanyl. A bicyclic cycloalkyl can be unsubstituted or independently substituted with one or more of the following groups: halogen, —NH₂, (C₁-C₆alkyl)NH—, di(C₁-C₆alkyl)amino-, —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, (C₁-C₆alkoxy)carbonyl, —C(O)(C₁-C₆alkyl), C₆-C₁₄aryl, C₁-C₉heteroaryl, or C₃-C₈cycloalkyl, haloalkyl-, aminoalkyl-, —OC(O)(C₁-C₆alkyl), carboxyamidoalkyl-, or —NO₂. Additionally, each of any two hydrogen atoms on the same carbon atom of the bicyclic cycloalkyl rings can be replaced by an oxygen atom to form an oxo (═O) substituent or the two hydrogen atoms can be replaced by an alkylenedioxy group so that the alkylenedioxy group, when taken together with the carbon atom to which it is attached, form a 5- to 7-membered heterocycle containing two oxygen atoms.

“Carboxyamidoalkyl-” refers to a primary carboxyamide (CONH₂), a secondary carboxyamide (CONHR′) or a tertiary carboxyamide (CONR′R″), where R′ and R″ are the same or different substituent groups selected from C₁-C₆alkyl, C₂-C₆alkenyl, C₂-C₆alkynyl, C₆-C₁₄aryl, C₁-C₉heteroaryl, or C₃-C₈cycloalkyl, attached to the parent compound by an C₁-C₆alkylene group as defined above. Exemplary C₁-C₆carboxyamidoalkyl-groups include but are not limited to NH₂C(O)—CH₂—, CH₃NHC(O)—CH₂CH₂—, (CH₃)₂NC(O)—CH₂CH₂CH₂—, CH₂═CHCH₂NHC(O)—CH₂CH₂CH₂CH₂—, HCCCH₂NHC(O)—CH₂CH₂CH₂CH₂CH₂—, C₆H₅NHC(O)—CH₂CH₂CH₂CH₂CH₂CH₂—, 3-pyridylNHC(O)—CH₂CH(CH₃)CH₂CH₂—, and cyclopropyl-CH₂NHC(O)—CH₂CH₂C(CH₃)₂CH₂—.

“Cycloalkenyl” refers to non-aromatic carbocyclic rings with one or more carbon-to-carbon double bonds within the ring system, for example C₃-C₁₀cycloalkenyl. The “cycloalkenyl” may be a single ring or may be multi-ring. Multi-ring structures may be bridged or fused ring structures. A cycloalkenyl can be unsubstituted or independently substituted with one or more of the following groups: halogen, —NH₂, (C₁-C₆alkyl)NH—, di(C₁-C₆alkyl)amino-, —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆alkoxy, C₁-C₆alkyl, —C(O)OH, (C₁-C₆alkoxy)carbonyl, —C(O)(C₁-C₆alkyl), C₆-C₁₄aryl, C₁-C₉heteroaryl, or C₃-C₈cycloalkyl, C₁-C₆haloalkyl-, C₁-C₆aminoalkyl-, —OC(O)(C₁-C₆alkyl), C₁-C₆carboxyamidoalkyl-, or —NO₂ Additionally, each of any two hydrogen atoms on the same carbon atom of the C₃-C₁₀cycloalkenyl rings may be replaced by an oxygen atom to form an oxo (═O) substituent or the two hydrogen atoms may be replaced by an alkylenedioxy group so that the alkylenedioxy group, when taken together with the carbon atom to which it is attached, form a 5- to 7-membered heterocycle containing two oxygen atoms. Examples of C₃-C₁₀cycloalkenyls include, but are not limited to, cyclopropenyl, cyclobutenyl, cyclopentenyl, cyclohexenyl, 4,4a-octalin-3-yl, and cyclooctenyl.

“Di(alkyl)amino-” refers to a nitrogen atom attached to two alkyl groups, as defined above. Each alkyl group can be independently selected. Representative examples of an di(C₁-C₆alkyl)amino-group include, but are not limited to, —N(CH₃)₂, —N(CH₂CH₃)(CH₃), —N(CH₂CH₃)₂, —N(CH₂CH₂CH₃)₂, —N(CH₂CH₂CH₂CH₃)₂, —N(CH(CH₃)₂)₂, —N(CH(CH₃)₂)(CH₃), —N(CH₂CH(CH₃)₂)₂, —NH(CH(CH₃)CH₂CH₃)₂, —N(C(CH₃)₃)₂, —N(C(CH₃)₃)(CH₃), and —N(CH₃)(CH₂CH₃). The two alkyl groups on the nitrogen atom, when taken together with the nitrogen to which they are attached, can form a 3- to 7-membered nitrogen containing heterocycle wherein up to two of the carbon atoms of the heterocycle can be replaced with —N(R)—, —O—, or —S(O)ₚ—. R is hydrogen, C₁-C₆alkyl, C₃-C₈cycloalkyl, C₆-C₁₄aryl, C₁-C₉heteroaryl, C₁-C₆aminoalkyl-, or arylamino. Variable p is 0, 1, or 2.

“Halo” or “halogen” is —F, —Cl, —Br, or —I.

“Haloalkyl-” refers to a alkyl group, as defined above, wherein one or more of the hydrogen atoms has been replaced with —F, —Cl, —Br, or —I. Each substitution can be independently selected. Representative examples of an C₁-C₆haloalkyl-group include, but are not limited to, —CH₂F, —CCl₃, —CF₃, CH₂CF₃, —CH₂Cl, —CH₂CH₂Br, —CH₂CH₂I, —CH₂CH₂CH₂F, —CH₂CH₂CH₂Cl, —CH₂CH₂CH₂CH₂Br, —CH₂CH₂ CH₂CH₂I, —CH₂CH₂CH₂CH₂CH₂Br, —CH₂CH₂CH₂CH₂CH₂I, —CH₂CH(Br)CH₃, —CH₂CH(Cl)CH₂CH₃, —CH(F)CH₂CH₃ and —C(CH₃)₂(CH₂Cl).

“Heteroaryl” refers to 5-10-membered mono and bicyclic aromatic groups containing at least one heteroatom selected from oxygen, sulfur, and nitrogen. Examples of monocyclic C₁-C₉heteroaryl radicals include, but are not limited to, oxazinyl, thiazinyl, diazinyl, triazinyl, thiadiazolyl, tetrazinyl, imidazolyl, tetrazolyl, isoxazolyl, furanyl, furazanyl, oxazolyl, thiazolyl, thiophenyl, pyrazolyl, triazolyl, pyrimidinyl, N-pyridyl, 2-pyridyl, 3-pyridyl and 4-pyridyl. Examples of bicyclic heteroaryl radicals include but are not limited to, benzimidazolyl, indolyl, isoquinolinyl, benzofuranyl, benzothiophenyl, indazolyl, quinolinyl, quinazolinyl, purinyl, benzisoxazolyl, benzoxazolyl, benzthiazolyl, benzodiazolyl, benzotriazolyl, isoindolyl, and indazolyl. The contemplated heteroaryl rings or ring systems have a minimum of 5 members. Therefore, for example, C₁heteroaryl radicals would include but are not limited to tetrazolyl, C₂heteroaryl radicals include but are not limited to triazolyl, thiadiazolyl, and tetrazinyl, C₉heteroaryl radicals include but are not limited to quinolinyl and isoquinolinyl. A heteroaryl group can be unsubstituted or substituted with one or more of the following groups: C₁-C₆alkyl, halo, C₁-C₆haloalkyl-, hydroxyl, C₁-C₆hydroxylalkyl-, —NH₂, C₁-C₆aminoalkyl-, di(C₁-C₆alkyl)amino-, —COOH, —C(O)O—(C₁-C₆alkyl), OC(O)(C₁-C₆alkyl), N-alkylamido-, —C(O)NH₂, (C₁-C₆alkyl)amido-, or —NO₂.

“(C₁-C₉Heteroaryl)alkyl” refers to an alkyl group, as defined above, wherein one or more of the alkyl group's hydrogen atoms has been replaced with an heteroaryl group as defined above. Examples of (C₁-C₉heteroaryl)alkyl moieties include 2-pyridylmethyl, 2-thiophenylethyl, 3-pyridylpropyl, 2-quinolinylmethyl, 2-indolylmethyl, and the like. An (C₁-C₉heteroaryl)alkyl group can be unsubstituted or substituted with one or more of the following groups: halogen, —NH₂, hydroxyl, (C₁-C₆alkyl)NH—, di(C₁-C₆alkyl)amino-, —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆)(C₁-C₆alkyl)), —CN, hydroxyl, C₁-C₆alkoxy, C₁-C₆alkyl, —C(O)OH, (C₁-C₆alkoxy)carbonyl, —C(O)(C₁-C₆alkyl), C₆-C₁₄aryl, C₁-C₉heteroaryl, C₃-C₈cycloalkyl, C₁-C₆haloalkyl-, C₁-C₆aminoalkyl-, —OC(O)(C₁-C₆alkyl), C₁-C₆carboxyamidoalkyl-, or —NO₂.

“(Heteroaryl)oxy” refers to the group Het-O— where Het is a heteroaryl group, as defined above. Exemplary (C₁-C₉heteroaryl)oxy groups include but are not limited to pyridin-2-yloxy, pyridin-3-yloxy, pyrimidin-4-yloxy, and oxazol-5-yloxy. A (heteroaryl)oxy group can be unsubstituted or substituted with one or more of the following groups: C₁-C₆alkyl, halo, C₁-C₆haloalkyl-, hydroxyl, C₁-C₆hydroxylalkyl-, —NH₂, C₁-C₆aminoalkyl-, di(C₁-C₆alkyl)amino-, —COOH, —C(O)O—(C₁-C₆alkyl), —OC(O)(C₁-C₆alkyl), N-alkylamido-, —C(O)NH₂, (C₁-C₆alkyl)amido-, or —NO₂.

The term “heteroatom” refers to a sulfur, nitrogen, or oxygen atom.

“Heterocycle” or “heterocyclyl” refers to 3-10-membered monocyclic, fused bicyclic, and bridged bicyclic groups containing at least one heteroatom selected from oxygen, sulfur, and nitrogen. A heterocycle may be saturated or partially saturated. Exemplary C₁-C₉heterocyclyl groups include but are not limited to aziridine, oxirane, oxirene, thiirane, pyrroline, pyrrolidine, dihydrofuran, tetrahydrofuran, dihydrothiophene, tetrahydrothiophene, dithiolane, piperidine, 1,2,3,6-tetrahydropyridine-1-yl, tetrahydropyran, pyran, thiane, thiine, piperazine, oxazine, 5,6-dihydro-4H-1,3-oxazin-2-yl, 2,5-diazabicyclo[2.2.1]heptane, 2,5-diazabicyclo[2.2.2]octane, 3,6-diazabicyclo[3.1.1]heptane, 3,8-diazabicyclo[3.2.1]octane, 6-oxa-3,8-diazabicyclo[3.2.1]octane, 7-oxa-2,5-diazabicyclo[2.2.2]octane, 2,7-dioxa-5-azabicyclo[2.2.2]octane, 2-oxa-5-azabicyclo[2.2.1]heptane, 2-oxa-5-azabicyclo[2.2.2]octane, 3,6-dioxa-8-azabicyclo[3.2.1]octane, 3-oxa-6-azabicyclo[3.1.1]heptane, 3-oxa-8-azabicyclo[3.2.1]octane, 5,7-dioxa-2-azabicyclo[2.2.2]octane, 6,8-dioxa-3-azabicyclo[3.2.1]octane, 6-oxa-3-azabicyclo[3.1.1]heptane, 8-oxa-3-azabicyclo[3.2.1]octane, 8-oxa-3-azabicyclo[3.2.1]octan-3-yl, 2-methyl-2,5-diazabicyclo[2.2.1]heptane-5-yl, 1,3,3-trimethyl-6-azabicyclo[3.2.1]oct-6-yl, 4-methyl-3,4-dihydro-2H-1,4-benzoxazin-7-yl, thiazine, dithiane, and dioxane. The contemplated heterocycle rings or ring systems have a minimum of 3 members. Therefore, for example, C₁heterocyclyl radicals would include but are not limited to oxaziranyl, diaziridinyl, and diazirinyl, C₂heterocyclyl radicals include but are not limited to aziridinyl, oxiranyl, and diazetidinyl, C₉heterocyclyl radicals include but are not limited to azecanyl, tetrahydroquinolinyl, and perhydroisoquinolinyl.

“Heterocyclyl(alkyl)” refers to an alkyl group, as defined above, wherein one or more of the alkyl group's hydrogen atoms has been replaced with a heterocycle group as defined above. Heterocyclyl(C₁-C₆alkyl) moieties include 2-pyridylmethyl, 1-piperazinylethyl, 4-morpholinylpropyl, 6-piperazinylhexyl, and the like. A heterocyclyl(alkyl) group can be unsubstituted or substituted with one or more of the following groups: halogen, —NH₂, (C₁-C₆alkyl)NH—, di(C₁-C₆alkyl)amino-, —N(C₁-C₃alkyl) C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, (C₁-C₆alkoxy)carbonyl, —C(O)(C₁-C₆alkyl), 4- to 7-membered monocyclic heterocycle, C₆-C₁₄aryl, C₁-C₉heteroaryl, or C₃-C₈cycloalkyl.

“Hydroxylalkyl-” refers to a alkyl group, as defined above, wherein one or more of the C₁-C₆alkyl group's hydrogen atoms has been replaced with hydroxyl groups. Examples of C₁-C₆hydroxylalkyl-moieties include, for example, —CH₂OH, —CH₂CH₂OH, —CH₂CH₂CH₂OH, —CH₂CH(OH)CH₂OH, —CH₂CH(OH)CH₃, —CH(CH₃)CH₂OH, and higher homologs.

“Hydroxylalkenyl-” refers to an alkenyl group, defined above, and substituted on one or more sp³ carbon atoms with a hydroxyl group. Examples of C₃-C₆hydroxylalkenyl-moieties include chemical groups such as —CH═CHCH₂OH, —CH(CH═CH₂)OH, —CH₂CH═CHCH₂OH, —CH(CH₂CH═CH₂)OH, —CH═CHCH₂CH₂OH, —CH(CH═CHCH₃)OH, —CH═CHCH(CH₃)OH, —CH₂CH(CH═CH₂)OH, and higher homologs.

“Nitrogen-containing heteroaryl” refers to 5-10-membered mono and bicyclic aromatic groups containing at least one nitrogen atom and optionally additional heteroatoms selected from oxygen and sulfur. Examples of nitrogen-containing monocyclic C₁-C₉heteroaryl radicals include, but are not limited to, oxazinyl, thiazinyl, diazinyl, triazinyl, tetrazinyl, imidazolyl, tetrazolyl, isoxazolyl, furazanyl, oxazolyl, thiazolyl, pyrazolyl, triazolyl, pyrimidinyl, N-pyridyl, 2-pyridyl, 3-pyridyl and 4-pyridyl. Examples of nitrogen-containing bicyclic C₁-C₉heteroaryl radicals include but are not limited to, benzimidazolyl, indolyl, isoquinolinyl, indazolyl, quinolinyl, quinazolinyl, purinyl, benzisoxazolyl, benzoxazolyl, benzthiazolyl, benzodiazolyl, benzotriazolyl, isoindolyl and indazolyl. A nitrogen-containing C₁-C₉heteroaryl group can be unsubstituted or substituted with one or more of the following groups: C₁-C₆alkyl, halo, C₁-C₆haloalkyl-, hydroxyl, C₁-C₆hydroxylalkyl-, —NH₂, C₁-C₆aminoalkyl-, di(C₁-C₆alkyl)amino-, —COOH, —C(O)O—(C₁-C₆alkyl), —OC(O)(C₁-C₆alkyl), N-alkylamido-, —C(O)NH₂, (C₁-C₆alkyl)amido-, or —NO₂.

“Perfluoroalkyl-” refers to alkyl group, defined above, having two or more fluorine atoms. Examples of a C₁-C₆ perfluoroalkyl-group include CF₃, CH₂CF₃, CF₂CF₃, and CH(CF₃)₂.

The term “optionally substituted” as used herein means that at least one hydrogen atom of the optionally substituted group has been substituted with halogen, —NH₂, (C₁-C₆alkyl)NH—, di(C₁-C₆alkyl)amino-, —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆alkoxy, C₁-C₆alkyl, —C(O)OH, (C₁-C₆alkoxy)carbonyl, —C(O)(C₁-C₆alkyl), C₆-C₁₄aryl C₁-C₉heteroaryl, or C₃-C₈cycloalkyl.

A “subject” is a mammal, e.g., a human, mouse, rat, guinea pig, dog, cat, horse, cow, pig, or non-human primate, such as a monkey, chimpanzee, baboon or gorilla.

The compounds of the present invention exhibit an mTOR inhibitory activity and, therefore, can be utilized to inhibit abnormal cell growth in which mTOR plays a role. Thus, the compounds of the present invention are effective in the treatment of disorders with which abnormal cell growth actions of mTOR are associated, such as restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, cancer, etc. In particular, the compounds of the present invention possess excellent cancer cell growth inhibiting effects and are effective in treating cancers, preferably all types of solid cancers and malignant lymphomas, and especially, leukemia, skin cancer, bladder cancer, breast cancer, uterus cancer, ovary cancer, prostate cancer, lung cancer, colon cancer, pancreas cancer, renal cancer, gastric cancer, brain tumor, advanced renal cell carcinoma, acute lymphoblastic leukemia, malignant melanoma, soft-tissue or bone sarcoma, etc.

The compounds of the present invention exhibit a PI3 kinase inhibitory activity and, therefore, can be utilized in order to inhibit abnormal cell growth in which PI3 kinases play a role. Thus, the compounds of the present invention are effective in the treatment of disorders with which abnormal cell growth actions of PI3 kinases are associated, such as restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, cancer, etc. In particular, the compounds of the present invention possess excellent cancer cell growth inhibiting effects and are effective in treating cancers, preferably all types of solid cancers and malignant lymphomas, and especially, leukemia, skin cancer, bladder cancer, breast cancer, uterus cancer, ovary cancer, prostate cancer, lung cancer, colon cancer, pancreas cancer, renal cancer, gastric cancer, brain tumor, advanced renal cell carcinoma, acute lymphoblastic leukemia, malignant melanoma, soft-tissue or bone sarcoma, etc.

For therapeutic use, the pharmacologically active compounds of Formula I will normally be administered as a pharmaceutical composition comprising as the (or an) essential active ingredient at least one such compound in association with a solid or liquid pharmaceutically acceptable carrier and, optionally, with pharmaceutically acceptable adjutants and excipients employing standard and conventional techniques.

The pharmaceutical compositions of this invention include suitable dosage forms for oral, parenteral (including subcutaneous, intramuscular, intradermal and intravenous) bronchial or nasal administration. Thus, if a solid carrier is used, the preparation may be made into tablets, placed in a hard gelatin capsule in powder or pellet form, or in the form of a troche or lozenge. The solid carrier may contain conventional excipients such as binding agents, fillers, lubricants used to make tablets, disintegrants, wetting agents and the like. The tablet may, if desired, be film coated by conventional techniques. If a liquid carrier is employed, the preparation may be in the form of a syrup, emulsion, soft gelatin capsule, sterile vehicle for injection, an aqueous or non-aqueous liquid suspension, or may be a dry product for reconstitution with water or other suitable vehicle before use. Liquid preparations may contain conventional additives such as suspending agents, emulsifying agents, wetting agents, non-aqueous vehicle (including edible oils), preservatives, as well as flavoring and/or coloring agents. For parenteral administration, a vehicle normally will comprise sterile water, at least in large part, although saline solutions, glucose solutions and like may be utilized. Injectable suspensions also may be used, in which case conventional suspending agents may be employed. Conventional preservatives, buffering agents and the like also may be added to the parenteral dosage forms. Particularly useful is the administration of a compound of Formula I directly in parenteral formulations. The pharmaceutical compositions are prepared by conventional techniques appropriate to the desired preparation containing appropriate amounts of the active ingredient, that is, the compound of Formula I according to the invention. See, for example, Remington: The Science and Practice of Pharmacy, 20th Edition. Baltimore, Md.: Lippincott Williams & Wilkins, 2000.

The dosage of the compounds of Formula I to achieve a therapeutic effect will depend not only on such factors as the age, weight and sex of the patient and mode of administration, but also on the degree of potassium channel activating activity desired and the potency of the particular compound being utilized for the particular disorder of disease concerned. It is also contemplated that the treatment and dosage of the particular compound may be administered in unit dosage form and that one skilled in the art would adjust the unit dosage form accordingly to reflect the relative level of activity. The decision as to the particular dosage to be employed (and the number of times to be administered per day is within the discretion of the physician, and may be varied by titration of the dosage to the particular circumstances of this invention to produce the desired therapeutic effect.

A suitable dose of a compound of Formula I or pharmaceutical composition thereof for a mammal, including man, suffering from, or likely to suffer from any condition as described herein is an amount of active ingredient from about 0.01 .mg/kg to 10 mg/kg body weight. For parenteral administration, the dose may be in the range of 0.1 .mg/kg to 1 mg/kg body weight for intravenous administration. For oral administration, the dose may be in the range about 0.1 .mg/kg to 5 mg/kg body weight. The active ingredient will preferably be administered in equal doses from one to four times a day. However, usually a small dosage is administered, and the dosage is gradually increased until the optimal dosage for the host under treatment is determined.

However, it will be understood that the amount of the compound actually administered will be determined by a physician, in the light of the relevant circumstances including the condition to be treated, the choice of compound of be administered, the chosen route of administration, the age, weight, and response of the individual patient, and the severity of the patient's symptoms.

The amount of the compound of the present invention or a pharmaceutically acceptable salt thereof that is effective for inhibiting mTOR or PI3K in a subject. In addition, in vitro or in vivo assays can optionally be employed to help identify optimal dosage ranges. The precise dose to be employed can also depend on the route of administration, the condition, the seriousness of the condition being treated, as well as various physical factors related to the individual being treated, and can be decided according to the judgment of a health-care practitioner. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The number and frequency of dosages corresponding to a completed course of therapy will be determined according to the judgment of a health-care practitioner. The effective dosage amounts described herein refer to total amounts administered; that is, if more than one compound of the present invention or a pharmaceutically acceptable salt thereof is administered, the effective dosage amounts correspond to the total amount administered.

In one embodiment, the compound of the present invention or a pharmaceutically acceptable salt thereof is administered concurrently with another therapeutic agent.

In one embodiment, a composition comprising an effective amount of a compound of the present invention or a pharmaceutically acceptable salt thereof and an effective amount of another therapeutic agent within the same composition can be administered.

Effective amounts of the other therapeutic agents are well known to those skilled in the art. However, it is well within the skilled artisan's purview to determine the other therapeutic agent's optimal effective amount range. The compound of the present invention or a pharmaceutically acceptable salt thereof and the other therapeutic agent can act additively or, in one embodiment, synergistically. In one embodiment, of the invention, where another therapeutic agent is administered to an animal, the effective amount of the compound of the present invention or a pharmaceutically acceptable salt thereof is less than its effective amount would be where the other therapeutic agent is not administered. In this case, without being bound by theory, it is believed that the compound of the present invention or a pharmaceutically acceptable salt thereof and the other therapeutic agent act synergistically.

Procedures used to synthesize the compounds of the present invention are described in Schemes 1-61 and are illustrated in the examples. Reasonable variations of the described procedures, which would be evident to one skilled in the art, are intended to be within the scope of the present invention:

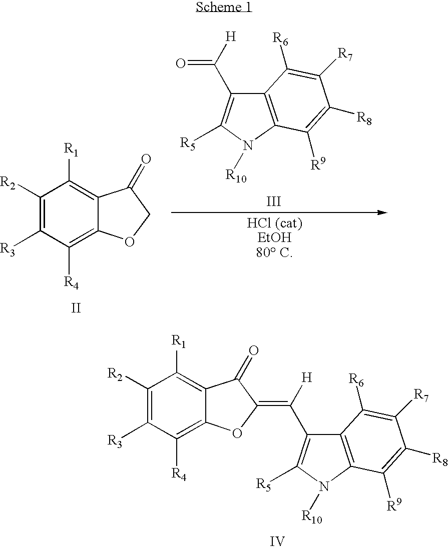

Benzofuranone molecules IV may be prepared according to Scheme 1 by reacting benzofuranone compounds II with heteroaryl aldehydes III in alcohols such as EtOH with catalytic amounts of an acid such as HCl, AcOH, or TFA at 80° C. Benzofuranone compounds II and heteroaryl aldehydes III can be purchased commercially or prepared synthetically via standard organic chemistry protocols.

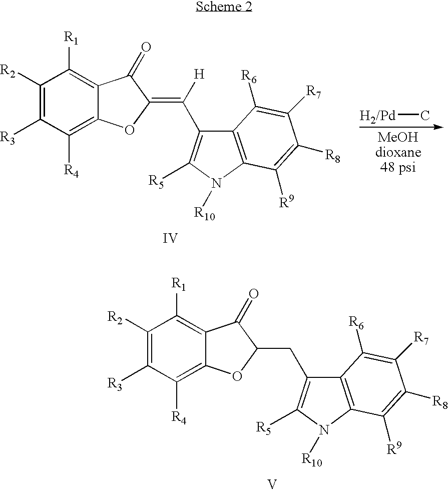

2-Methylbenzofuranone molecules V may be prepared according to Scheme 2 by reduction of 2-methylenebenzofuranones IV with Pd—C in MeOH/dioxane under 48 psi atmosphere of hydrogen.

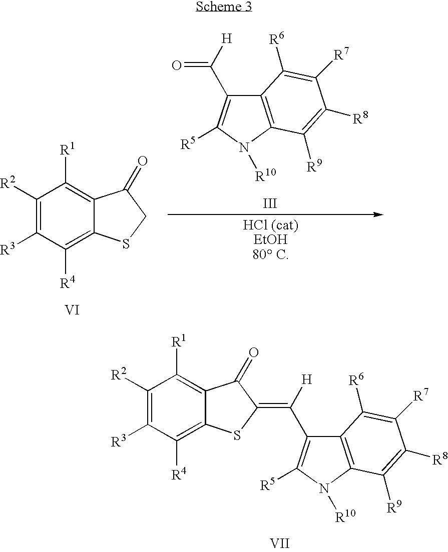

Benzothiophenone molecules VII may be prepared according to Scheme 3 by reacting benzothiophenone VI with the heteroaryl aldehydes III in a hydrocarbon solvent such as benzene with catalytic amounts of as base such as piperidine at 80° C. Benzothiophenone VI and heteroaryl aldehydes III can be purchased commercially or prepared synthetically via standard organic chemistry protocols.

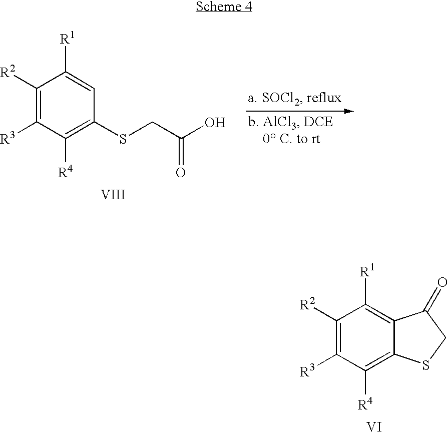

Benzothiophenone compounds VI as described in Scheme 4 can be obtained from the corresponding acids VIII using known literature procedures. To the acid (15.6 mmol) is added SOCl₂ (10 mL). After heating the resulting suspension to 85° C. for 1 hour, the reaction is concentrated in vacuo and placed under vacuum for 30 minutes. To the reaction is added methylene chloride (30 mL) and cooled on an ice-salt bath for 15 minutes. AlCl₃ (2.5 g) is added in portions over 20 minutes. The reaction is stirred with cooling for 15 minutes and then allowed to stir for 45 minutes at room temperature. The reaction is quenched with ice water, extracted with methylene chloride, and concentrated in vacuo to afford the desired compound without further purification.

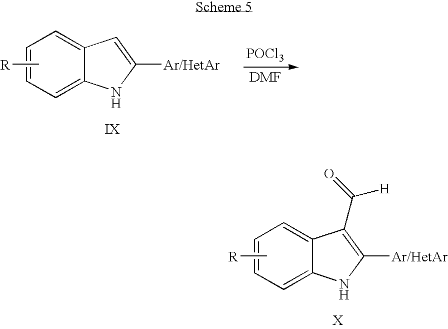

Several 3-Indole carboxaldehyde compounds as described in scheme 1 can be obtained commercially, while others can be synthesized using various synthetic methods outlined below. 3-Indole carboxaldehyde compounds as described by Scheme 5 can be obtained from the corresponding indole via reaction with POCl₃ under standard literature conditions.

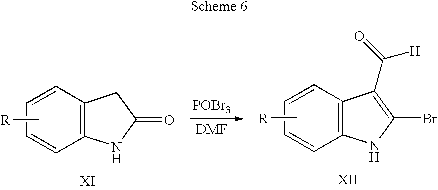

3-Indole carboxaldehyde compounds as described by Scheme 6 can be obtained from the corresponding oxindole via reaction with POBr₃ in DMF using literature procedures described in Arch. Pharmazie, 1972, 305, 523.

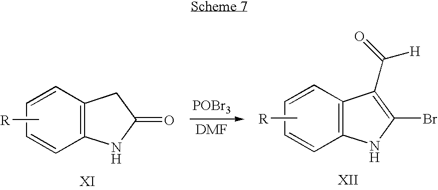

3-Indole carboxaldehyde compounds as described by Scheme 7 can be obtained from the corresponding indole via reaction with DMF/POCl₃ under standard literature conditions and then subsequent alkylation using alkyl halides and NaH in DMF under standard literature conditions.

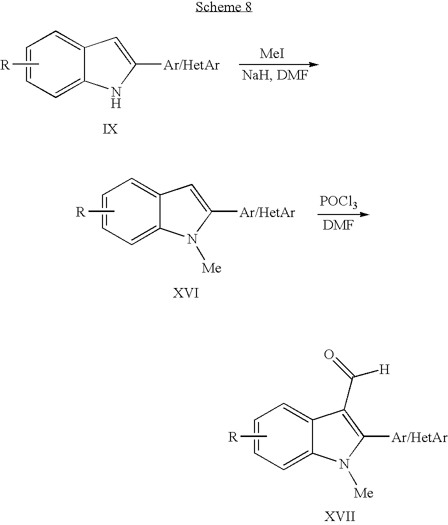

3-Indole carboxaldehyde compounds as described by Scheme 8 can be obtained from the corresponding indole via methylation using MeI and NaH in DMF under standard literature conditions and then subsequent reaction with POCl₃ under standard literature conditions.

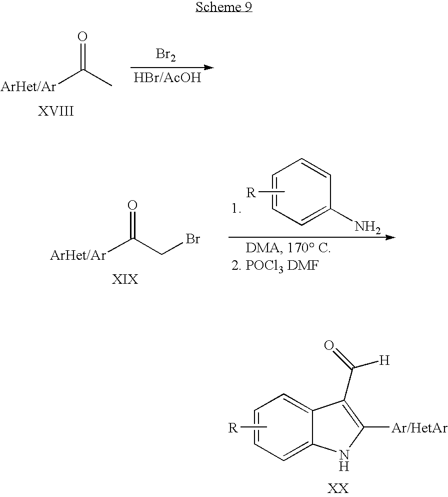

3-Indole carboxaldehyde compounds as described by Scheme 9 can be obtained from brominating the corresponding aryl or heteroaryl acetyl using procedure described in Austr. J. Chem. 1989, 42, 1735 then reacting the resulting the a-bromo ketone with anisidine as described in Bioorg. Med. Chem. 2002, 10, 3941 to afford the desired indole. The 3-indole carboxaldehyde derivative was then obtained via method 1.

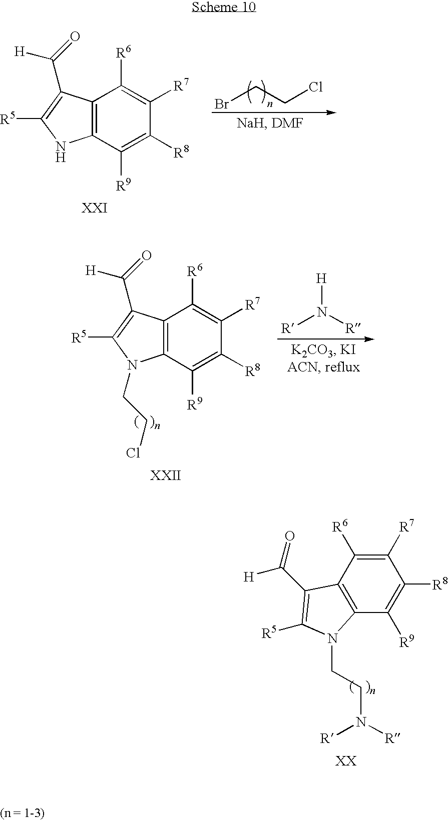

3-Indolecarboxaldehydes as described by Scheme 10 can be obtained by alkylation of the 3-indolecarboxaldehydes XXI using the corresponding ω-bromochloroalkanes and a base like NaH in a polar solvent like DMF under standard literature conditions. The resulting alkyl chloride XXII was then reacted with the desired secondary amine using potassium carbonate and potassium iodide in ACN at 80° C. under standard literature conditions.

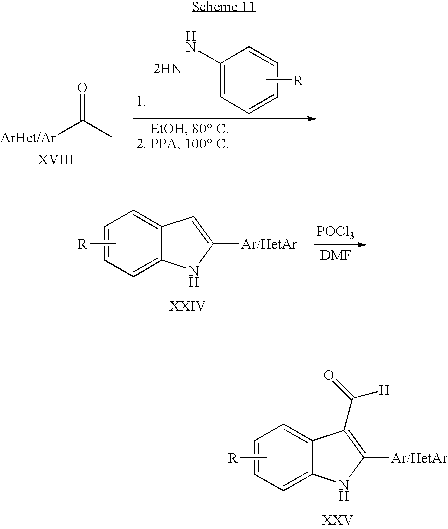

3-Indole carboxaldehyde compounds as described by Scheme 11 can be obtained from the corresponding ketone and hydrazine under standard Fischer-indole synthesis literature conditions.

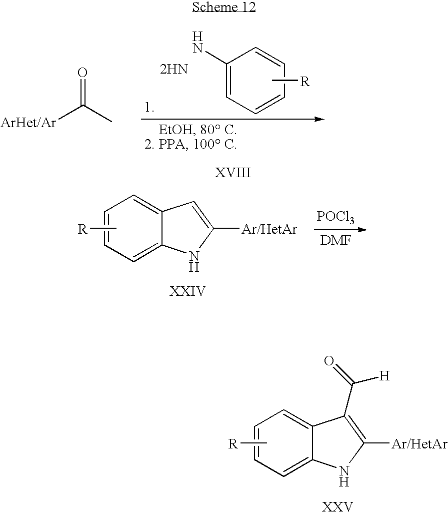

3-Indole carboxaldehyde compounds as described by Scheme 12 can be obtained from the corresponding indole via reaction with DMF/POCl₃ under standard literature conditions and then subsequent methylation using 2 equivalents of MeI and NaH in DMF under standard literature conditions.

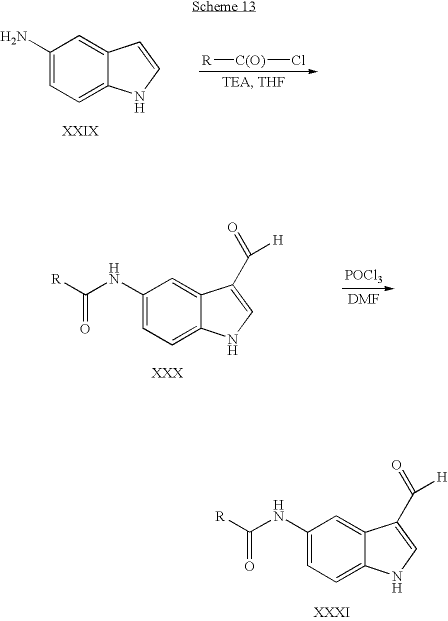

3-Indole carboxaldehyde compounds as described by Scheme 13 can be obtained from the corresponding indole via acylation with acid chlorides in THF in the presence of TEA under standard literature conditions and then subsequent reaction with DMF/POCl₃ under standard literature conditions.

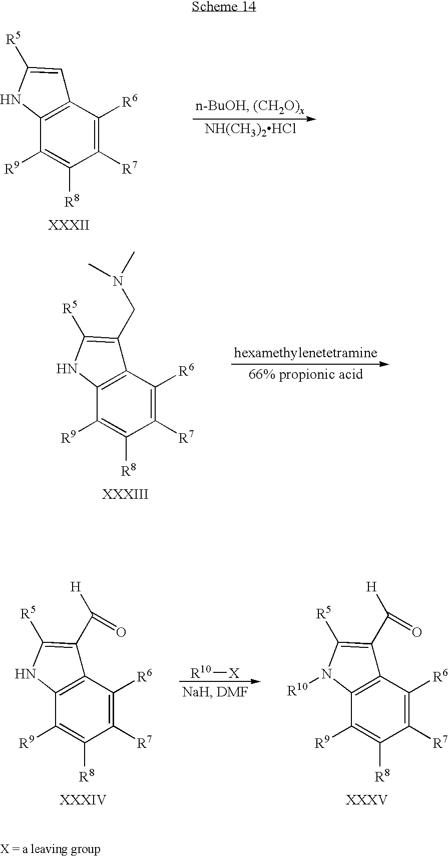

3-Indole carboxaldehyde compounds XXXV as described in Scheme 14 can be obtained by first generating gramine from indole XXXII, paraformaldehyde, and dimethylamine, by Mannich reaction followed by hydrolysis using literature procedures described in JACS 1955, 77, 457. This was followed by alkylation using R¹⁰—X and a base like NaH in an aprotic solvent like DMF under standard literature conditions.

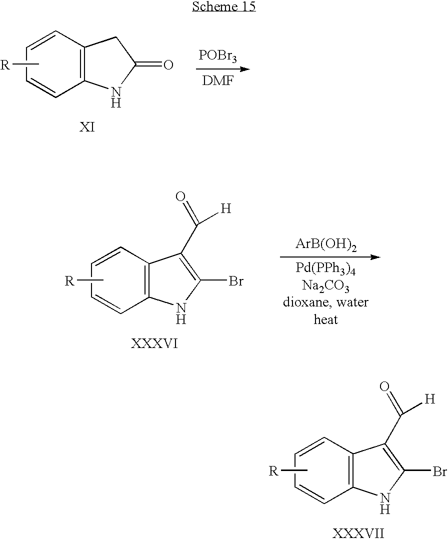

3-Indole carboxaldehyde compounds as described by Scheme 15 can be obtained from the corresponding oxindole via reaction with POBr₃ in DMF using literature procedures described in Arch. Pharmazie, 1972, 305, 523. The bromo derivative can be further subjected to a Suzuki coupling reaction with variety of boronic acids.

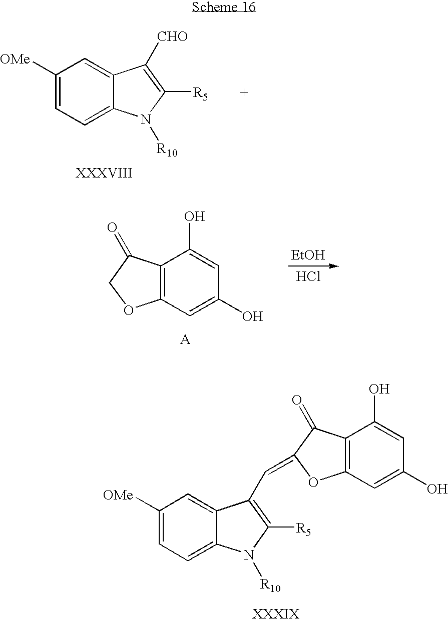

Condensation between 4,6-dihydroxy-benzofuran-3-one (A) and 5-methoxy-indole-3-carbaldehydes XXXVIII is shown in Scheme 16.

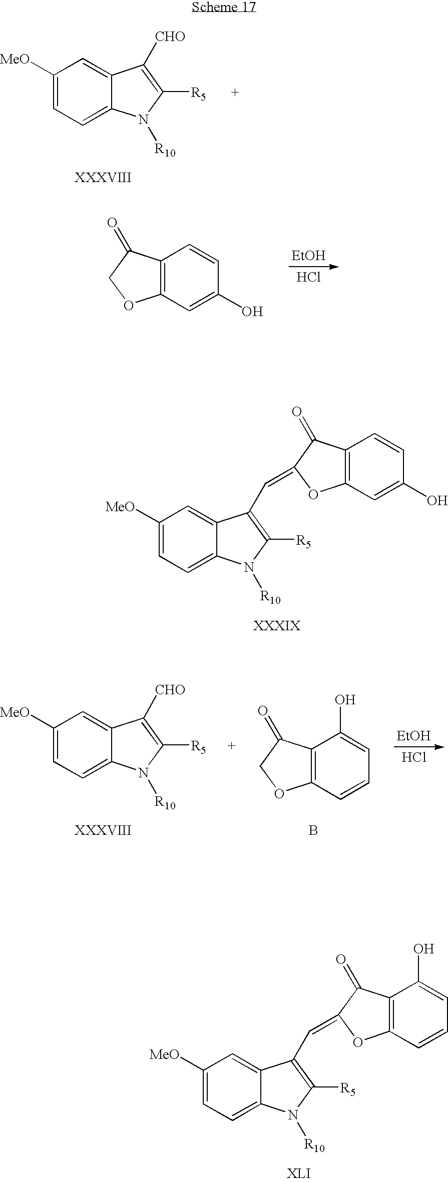

Condensation between mono-hydroxy-benzofuran-3-ones and 5-methoxy-indole-3-carbaldehydes, 6-mono-hydroxy derivatives and 4-mono-hydroxy derivatives is shown in Scheme 17.

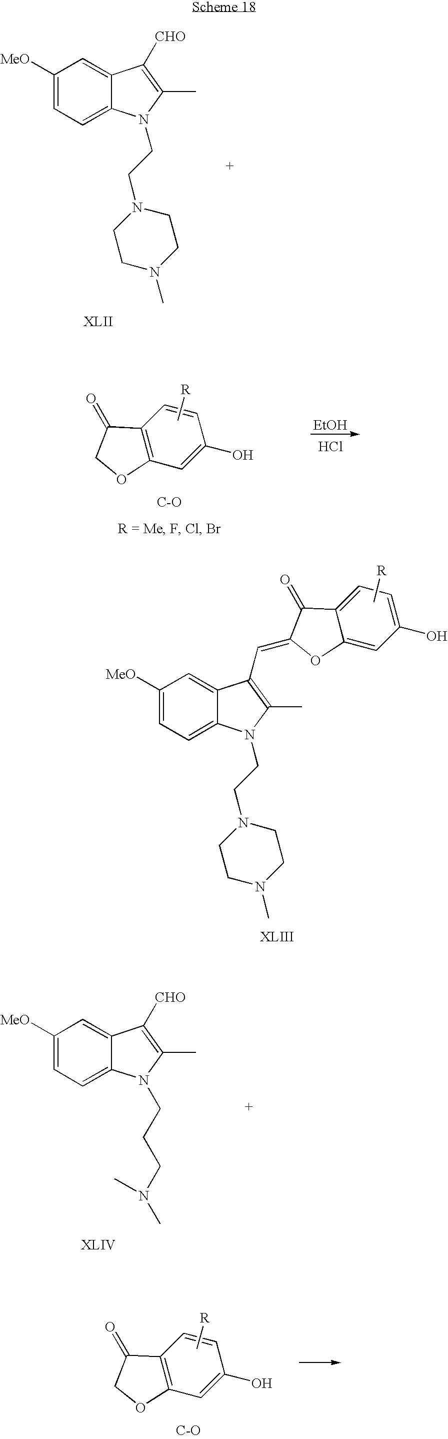

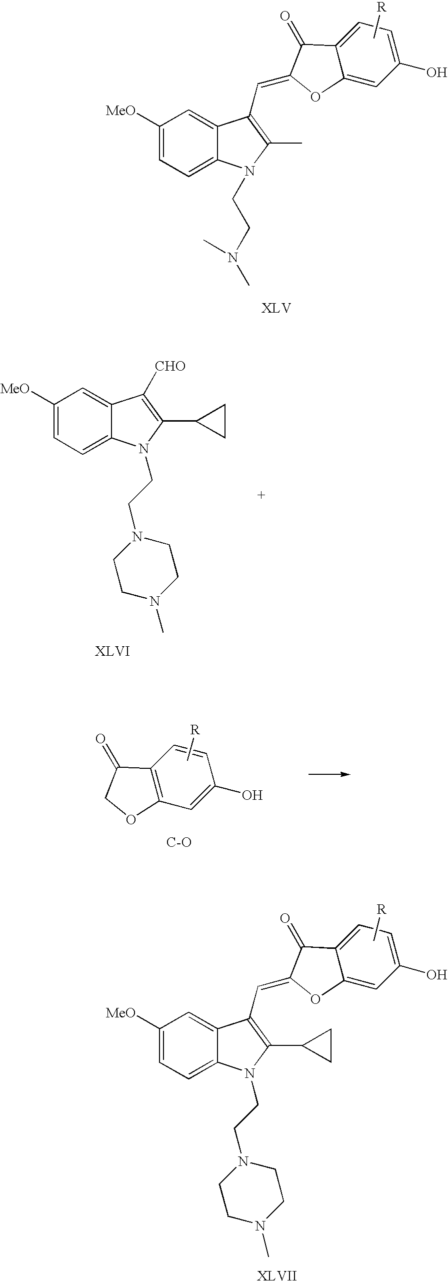

Condensation between substituted 6-hydroxy-benzofuranones and 5-methoxy-indole-3-carbaldehydes C-O is shown in Scheme 18.

Benzofuranone Compounds C-O

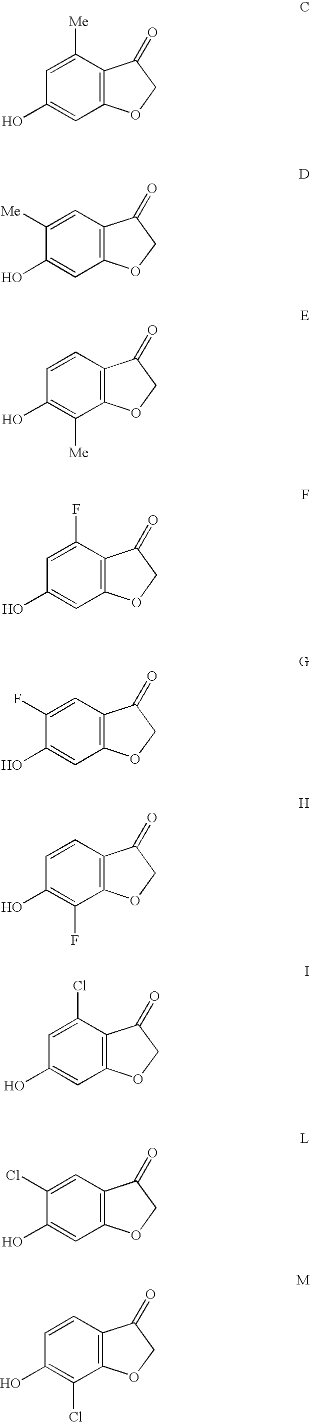

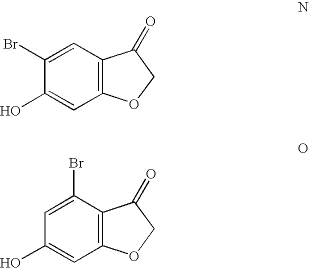

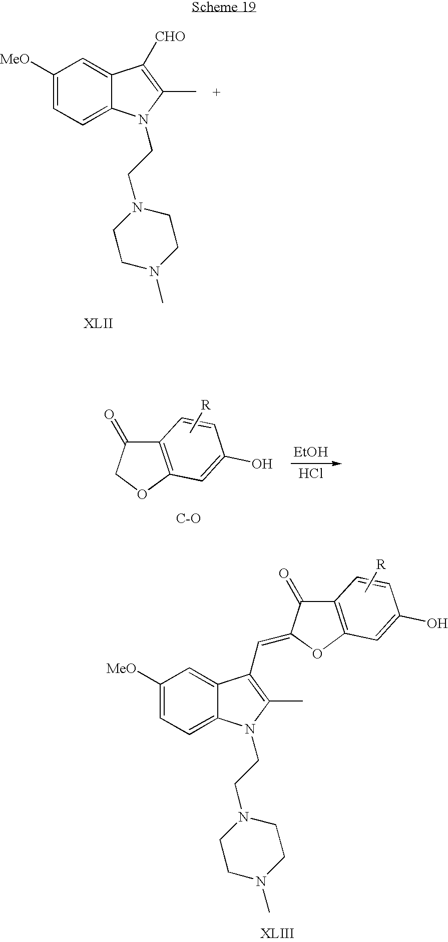

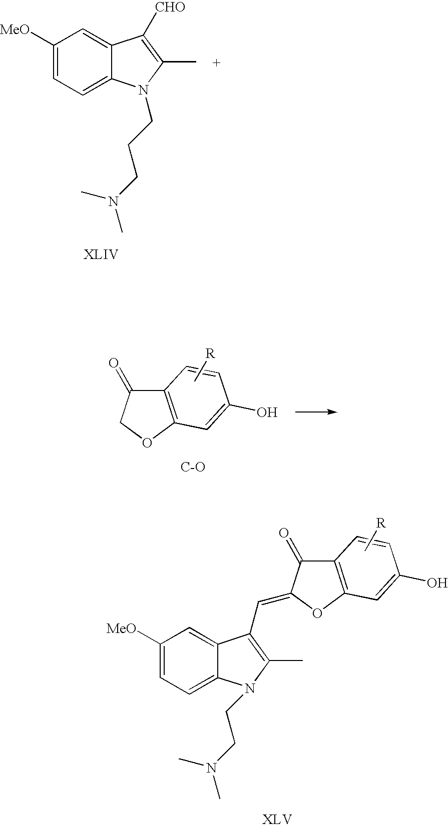

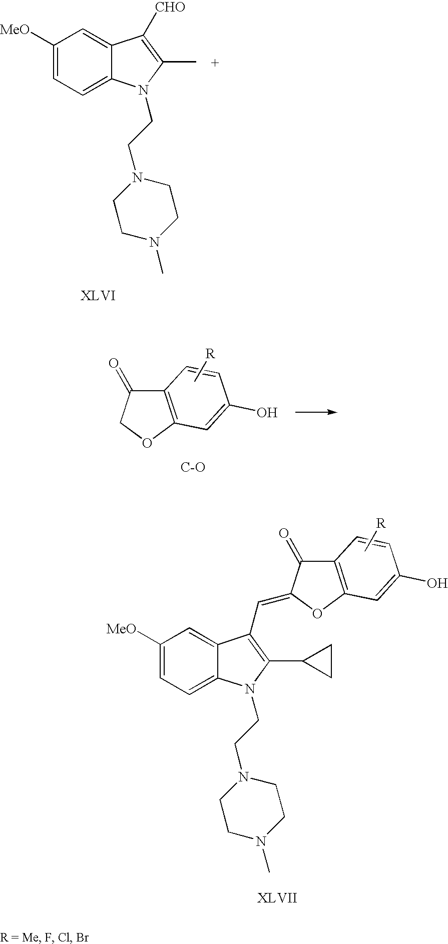

Preparation of (2Z)-2-[(4-aryl-1-methyl-1H-indol-3-yl)methylene]-4,6-dihydroxy-1-benzofuran-3(2H)-one compounds (LI) is shown in Scheme 19.

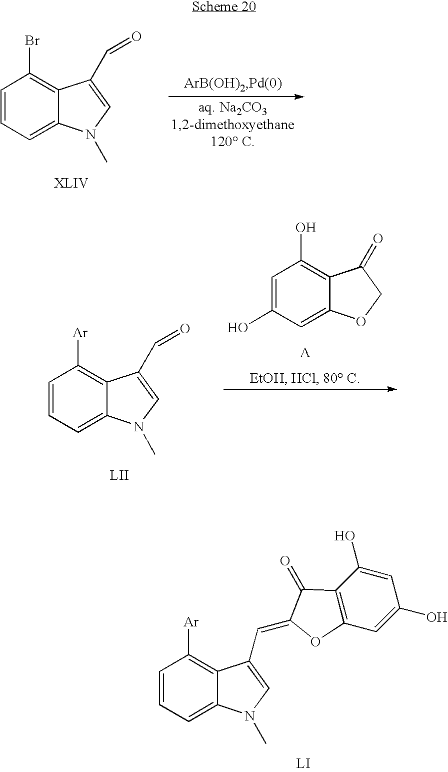

An alternative preparation of (2Z)-2[(4-aryl-1-methyl-1H-indol-3-yl)methylene]-4,6-dihydroxy-1-benzofuran-3(2H)-one (LI) is shown in Scheme 20.

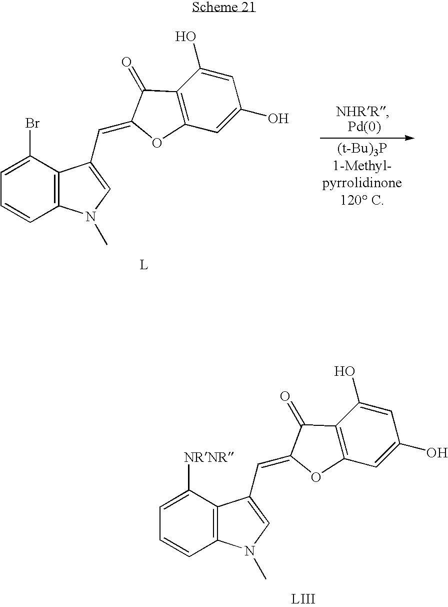

The preparation of (2Z)-2[(4-amino-1-methyl-1H-indol-3-yl)methylene]-4,6-dihydroxy-1-benzofuran-3(2H)-one (LII) is shown in Scheme 21.

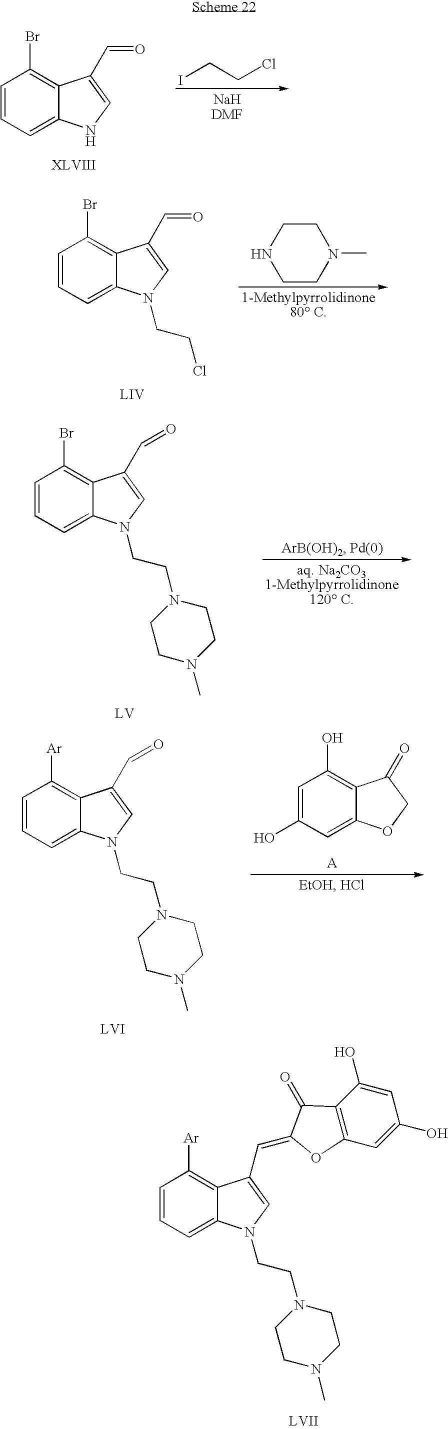

The preparation of (2Z)-2-({4-aryl-1-[2-(4-methylpiperazin-1-yl)ethyl]-1H-indol-3-yl}methylene)-4,6-dihydroxy-1-benzofuran-3(2H)-one compounds (LVII) is shown in Scheme 22.

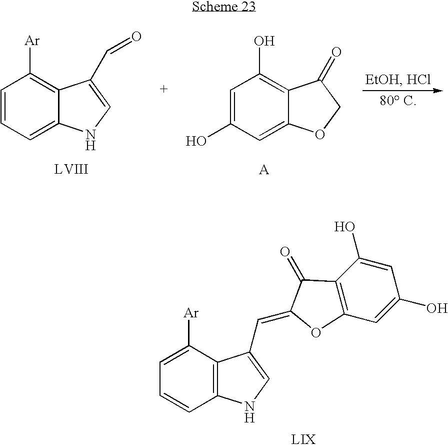

The preparation of (2Z)-2[(4-aryl-1H-indol-3-yl)methylene]-4,6-dihydroxy-1-benzofuran-3(2H)-one (LIX) is shown in Scheme 23.

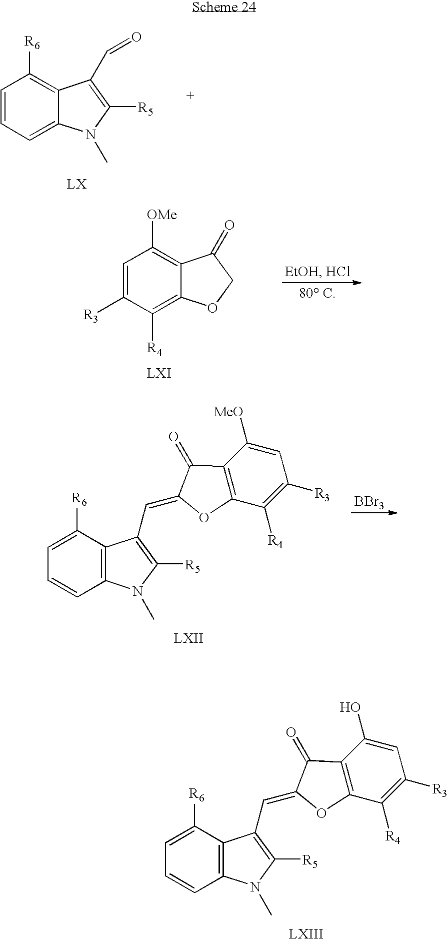

The preparation of (2Z)-2(-1H-indol-3-yl)methylene-4-methoxy-1-benzofuran-3(2H)-one (LXII) and its demethylation to (2Z)-2(-1H-indol-3-yl)methylene-4-hydroxy-1-benzofuran-3(2H)-one (LXIII) are shown in Scheme 24.

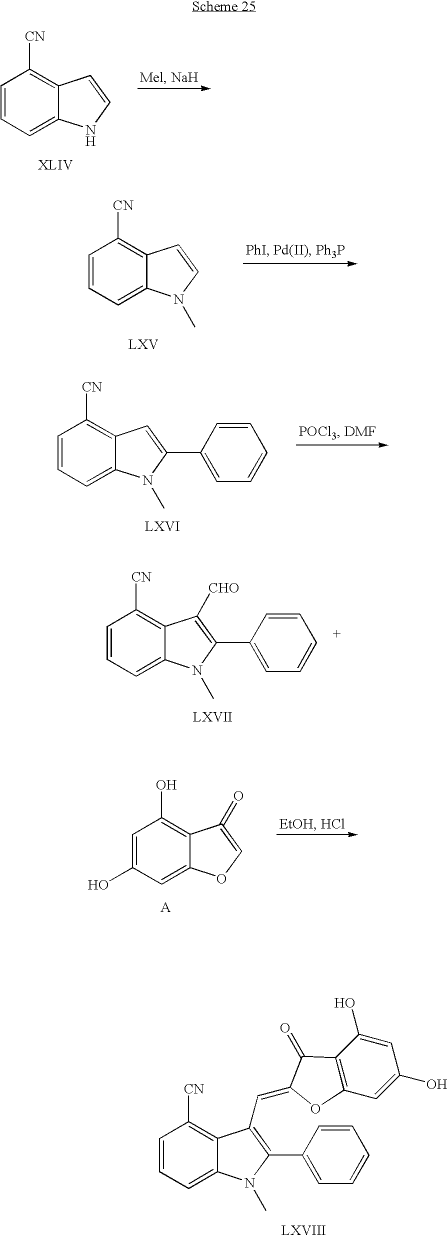

The preparation of 3-[(Z)-(4,6-dihydroxy-3-oxo-1-benzofuran-2(3H)-ylidene)methyl]-1-methyl-2-phenyl-1H-indole-4-carbonitrile (LXVIII) is shown in Scheme 25.

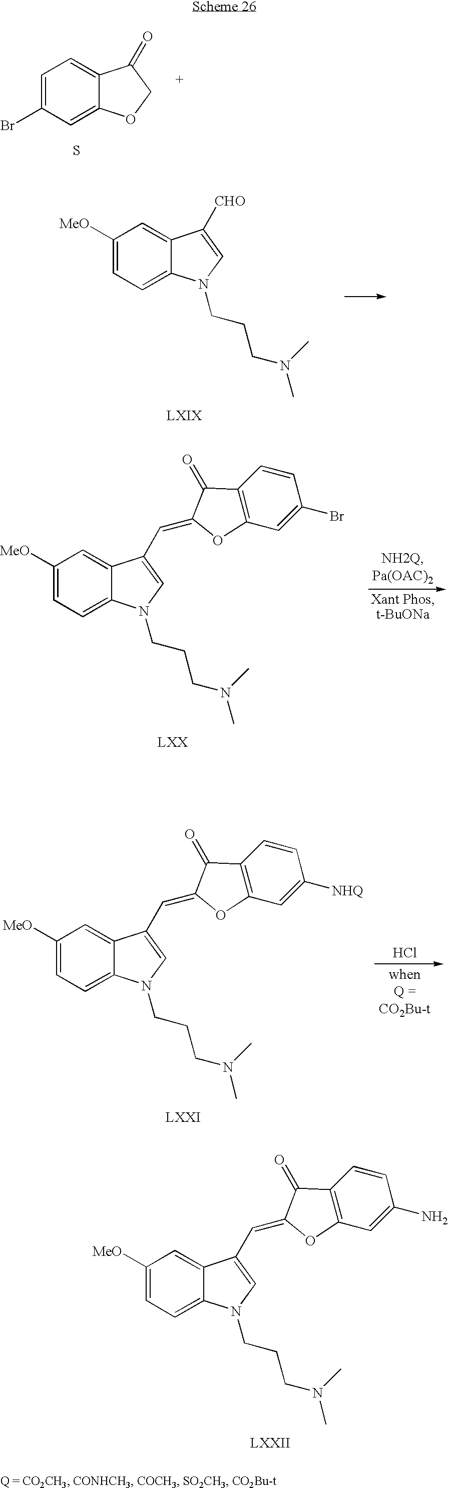

The preparation of 6-substituted (2Z)-2-({1-[3-(dimethylamino)propyl]-5-methoxy-1H-indol-3-yl}methylene)-1-benzofuran-3(2H)-one (22).

### Scheme 27

The preparation of (2Z)-2-({1-[3-(dimethylamino)propyl]-5-methoxy-1H-indol-3-yl}methylene)-6-(hydroxymethyl)-1-benzofuran-3(2H)-one (LXXIII) is shown in Scheme 27.

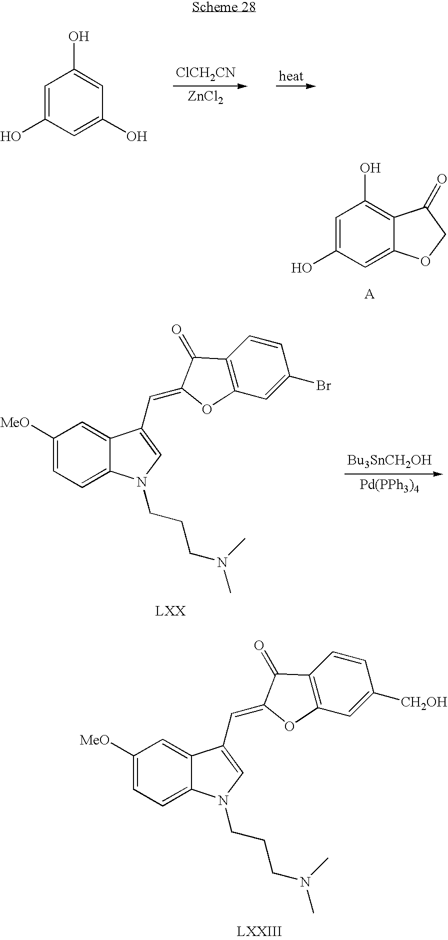

Preparation of 4,6-dihydroxybenzofuranone (Compound A) from phloroglucinol by thermal cyclization of the intermediate phenoxyacetonitrile, as shown in Scheme 28.

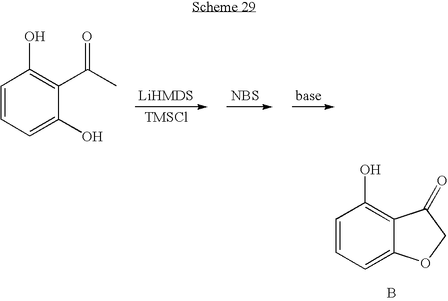

Preparation of 4-hydroxybenzofuranone (Compound B) from 1-(2,6-dihydroxyphenyl)ethanone by bromination of the enol ether followed by base-induced cyclization, as shown in Scheme 29.

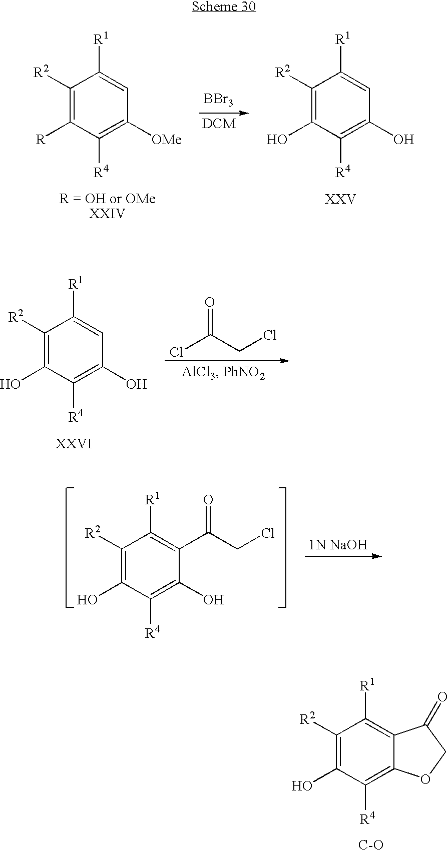

Preparation of monosubstituted 6-hydroxy benzofuranones (Compounds C-O) from anisole compounds LXXIV as shown in Scheme 30.

| Benzofuranone | R¹ | R² | R⁴ |
|---|---|---|---|
| C | Me | H | H |
| D | H | Me | H |
| E | H | H | Me |
| F | F | H | H |
| G | H | F | H |
| H | H | H | F |
| I | CI | H | H |
| L | H | Cl | H |
| M | H | H | Cl |
| N | H | Br | H |
| O | Br | H | H |

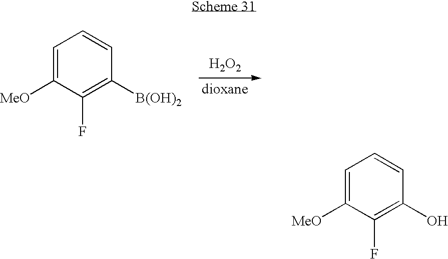

Preparation of 2-fluoro-3-methoxy-phenol as shown in Scheme 31.

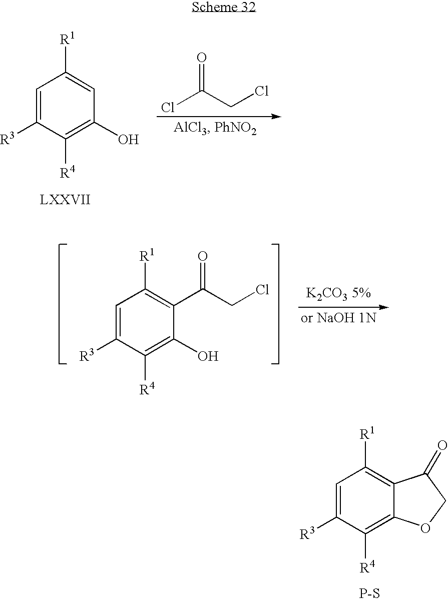

Preparation of other commercially non-available benzofuranone compounds (Compounds P-S) as shown in Scheme 32.

| Benzofuranone | R¹ | R³ | R⁴ |
|---|---|---|---|
| P | OMe | OMe | H |
| Q | H | H | Br |
| R | OMe | OH | H |
| S | H | Br | H |

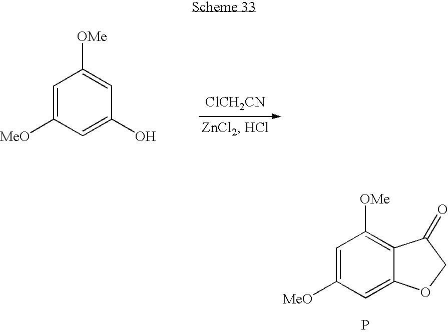

Preparation of 4,6-dimethoxybenzofuran-3(2H)-one (Compound P) as shown above in Scheme 33 by a one-step alkylation-cyclization process.

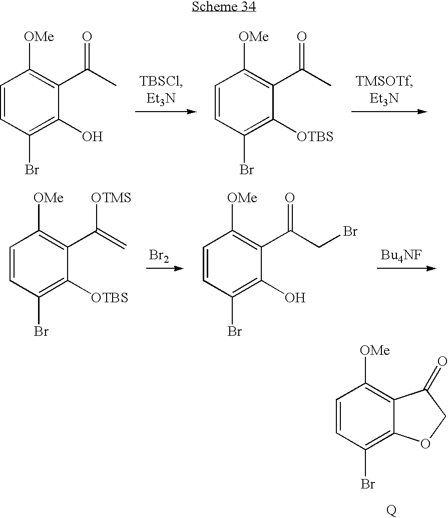

Preparation of 7-bromo-4-methoxybenzofuran-3(2H)-one (Compound Q) from 1-(3-bromo-2-hydroxy-6-methoxyphenyl)ethanone by bromination of the enol ether followed by fluoride-induced cyclization, as shown in Scheme 34.

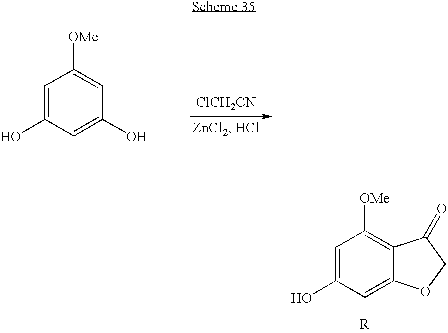

Preparation of 6-hydroxy-4-methoxybenzofuran-3(2H)-one (Compound R) as shown above in Scheme 35 by a one-step alkylation-cyclization process.

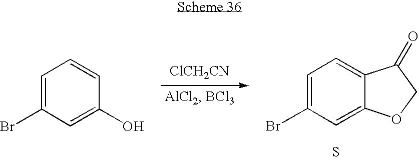

Preparation of 6-bromobenzofuran-3(2H)-one (Compound S) as shown above in Scheme 36 by another one-step alkylation-cyclization process.

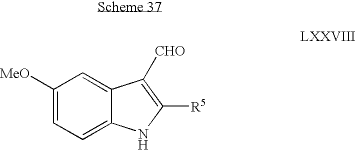

R⁵ substituent

| | |
|---|---|
| H | Me |
| a | b |
| 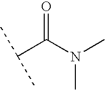 | 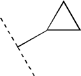 |
| c | d |
| CF₃ | 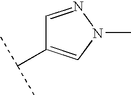 |
| e | f |
| 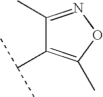 | 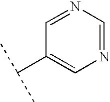 |
| g | h |
| 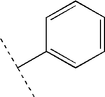 | 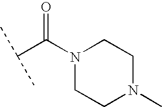 |
| i | j |
| 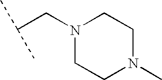 | 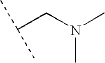 |
| k | l |
| 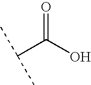 | 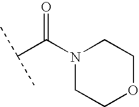 |
| m | n |
| 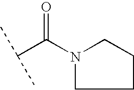 | 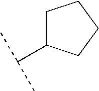 |
| o | p |
| 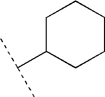 | 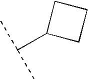 |
| q | r |

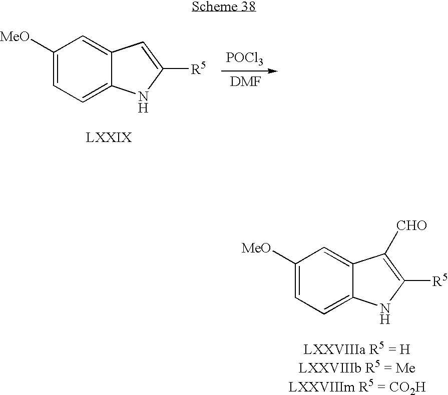

The preparation of 5-methoxy-indole-3-carbaldehyde (LXXVIIIa), 5-methoxy-2-methyl-indole-3-carbaldehyde (LXXVIIIb), and 3-formyl-5-methoxy-indole-2-carboxylic acid (LXXVIIIm) is shown in Scheme 38.

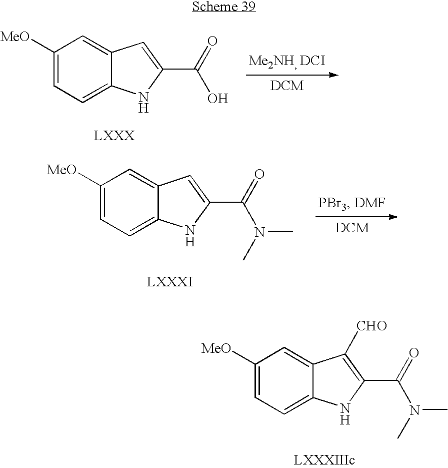

The preparation of 3-formyl-5-methoxy-indole-2-carboxylic acid dimethylamide (LXXVIIIc) is shown in Scheme 39.

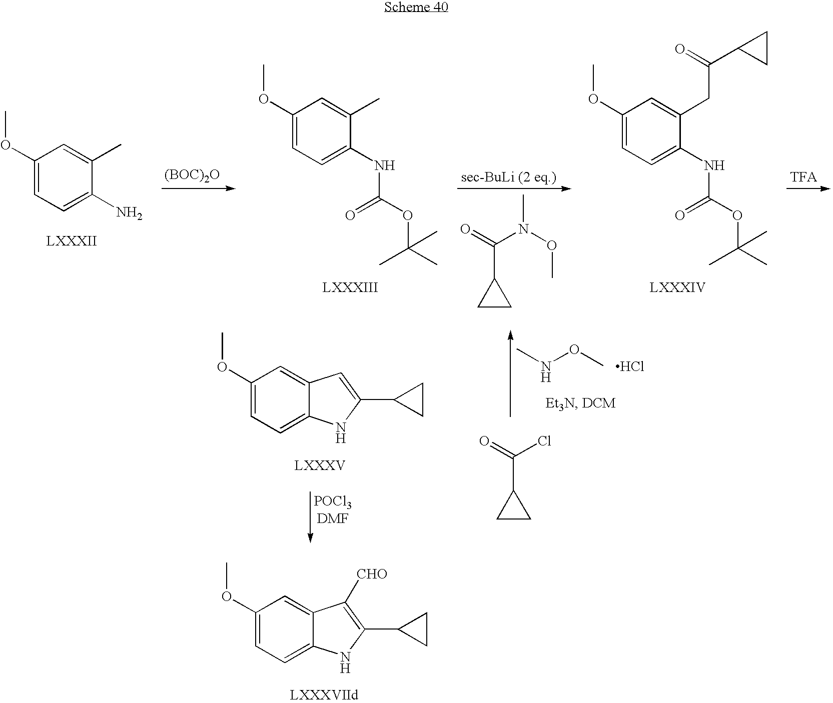

The preparation of 5-methoxy-2-cyclopropyl-indole-3-carbaldehyde (LXXVIIId) is shown in Scheme 40.

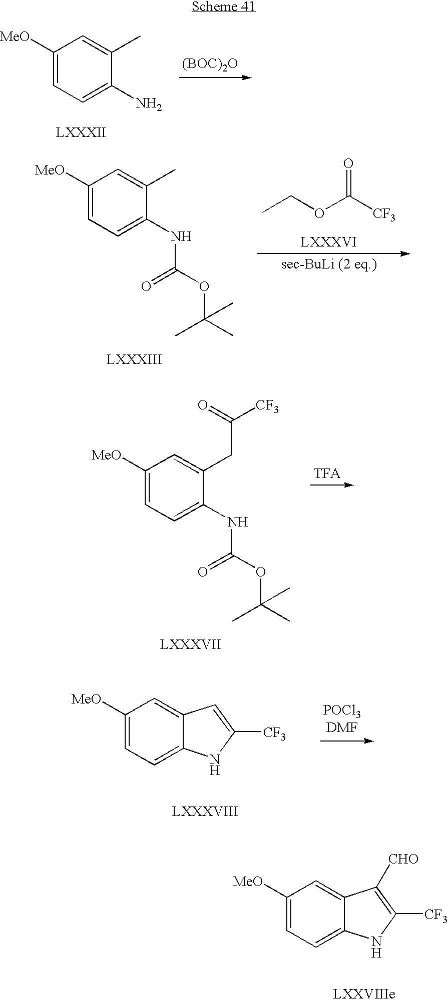

The preparation of 5-methoxy-2-trifluoromethyl-indole-3-carbaldehyde (LXXVIIIe) is shown in Scheme 41.

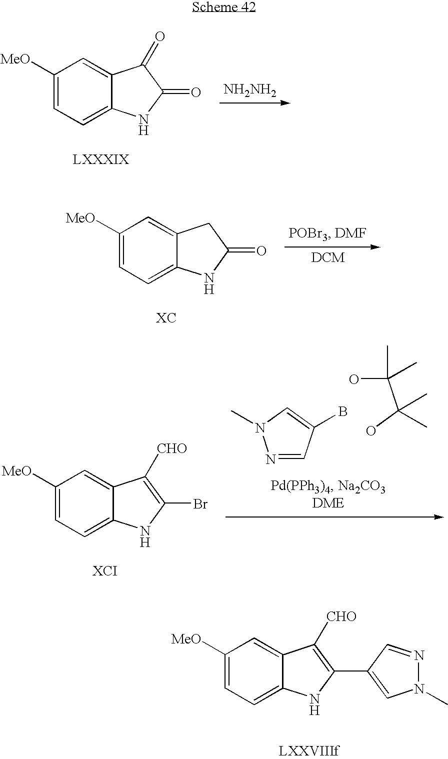

The preparation of 5-methoxy-2-(1-methyl-1H-pyrazol-4-yl)-indole-3-carbaldehyde (LXXVIIIf) is shown in Scheme 42.

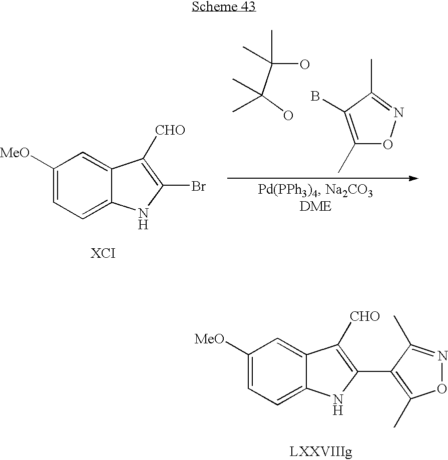

The preparation of 2-(3,5-Dimethyl-isoxazol-4-yl)-5-methoxy-indole-3-carbaldehyde (LXXVIIIIg) is shown in Scheme 43.

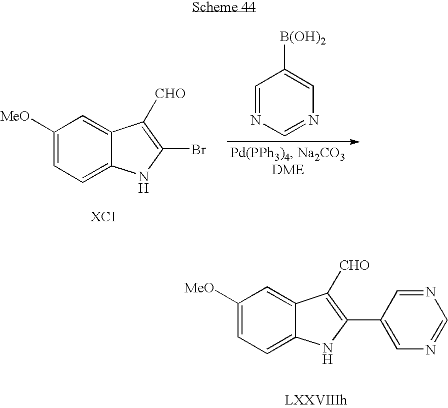

The preparation of 5-methoxy-2-pyrimidin-5-yl-indole-3-carbaldehyde (LXXVIIIh) is shown in Scheme 44.

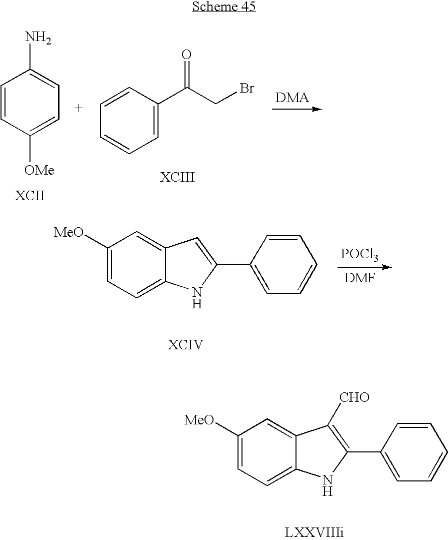

The preparation of 5-methoxy-2-phenyl-indole-3-carbaldehyde (LXXVIIIi) is shown in Scheme 45.

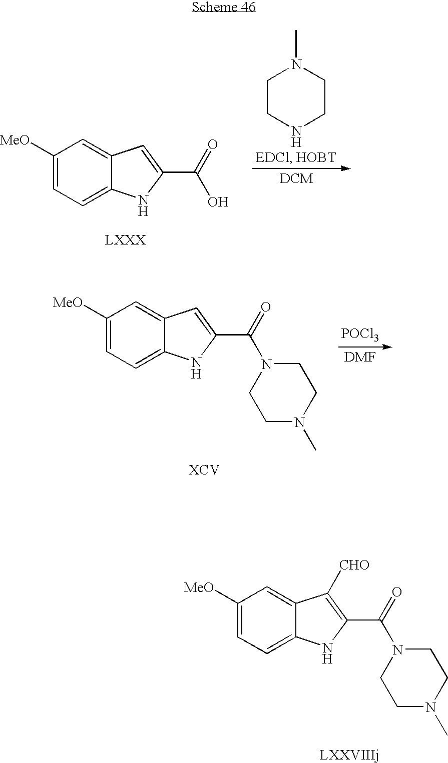

The preparation of 5-methoxy-2-(4-methyl-piperazine-1-carbonyl)-indole-3-carbaldehyde (LXXVIIIj) is shown in Scheme 46.

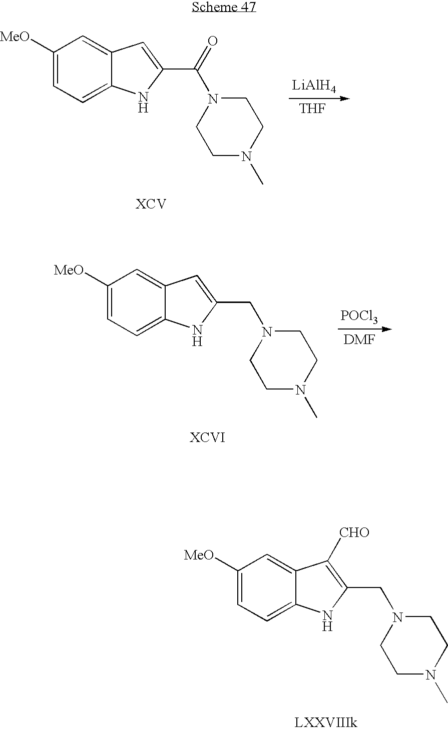

The preparation of 5-methoxy-2-(4-methyl-piperazin-1-ylmethyl)-indole-3-carbaldehyde (LXXVIIIk) is shown in Scheme 47.

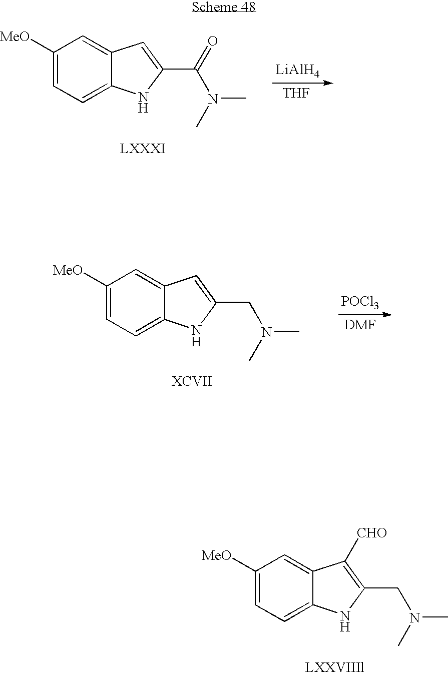

The preparation of 2-dimethylaminomethyl-5-methoxy-indole-3-carbaldehyde (LXXVIIII) is shown in Scheme 48.

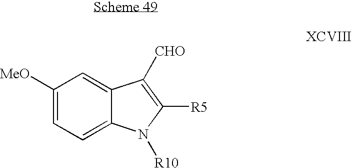

The synthesis of N-substituted 5-methoxy-indole-3-carbaldehydes (XCVIIIx-y) is summarized in Scheme 49.

R⁵ substituent

| | | |
|---|---|---|
| H | Me | 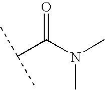 |
| a | b | c |
| 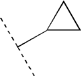 | CF₃ | 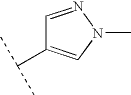 |
| d | e | f |
| 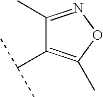 | 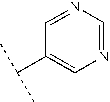 | 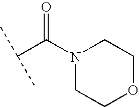 |
| g | h | n |
| 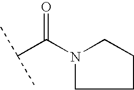 | 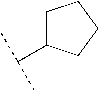 | 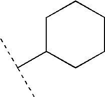 |
| o | p | q |

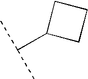

r

R¹⁰ substituent

| 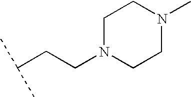 | 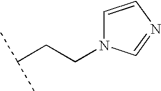 |
|---|---|
| 1 | 2 |
| 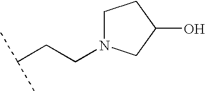 | 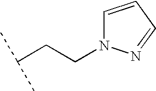 |
| 3 | 4 |
| 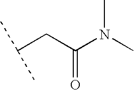 | 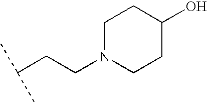 |
| 5 | 6 |
| 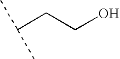 | 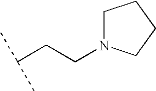 |
| 7 | 8 |
| 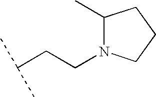 | 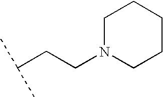 |
| 9 | 10 |
| 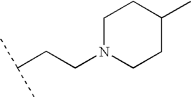 | 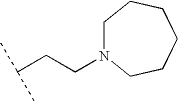 |
| 11 | 12 |
| 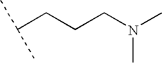 | 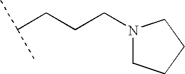 |
| 13 | 14 |
| 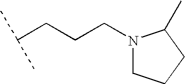 | 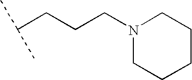 |
| 15 | 16 |
| 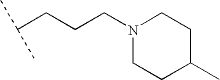 | 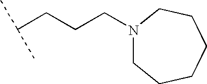 |
| 17 | 18 |

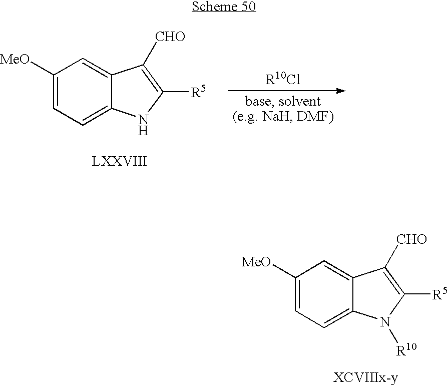

One route for the preparation of XCVIIIx-y is shown in Scheme 50.

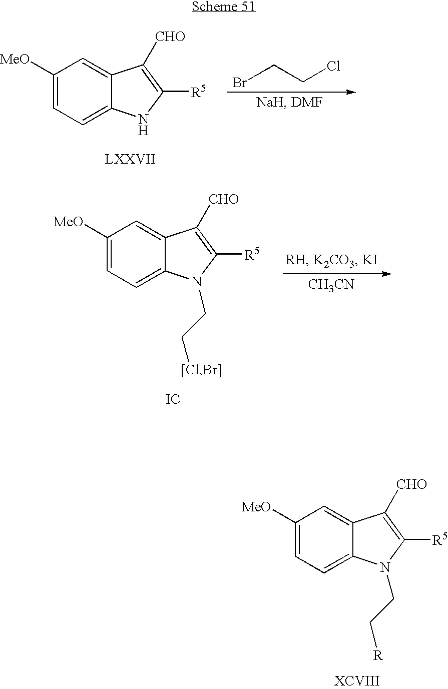

A dialkylation process was used to make the XCVIII compounds containing a heterocyclyl(ethylene) substituent as R¹⁰, as shown in Scheme 51.

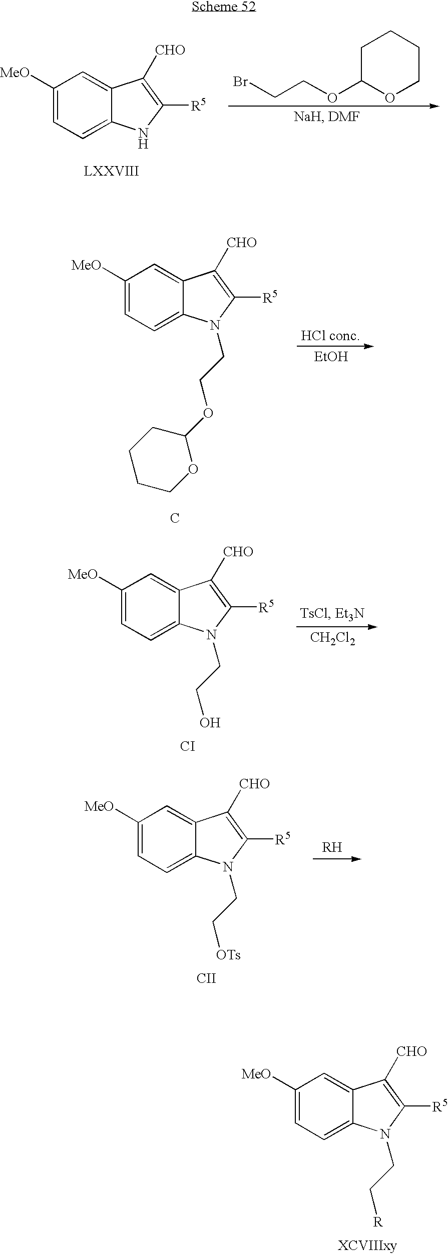

A dialkylation process was also used to make the XCVIII compounds containing a heterocyclyl(ethylene) substituent as R¹⁰ via a protected 2-bromoethanol reagent, as shown in Scheme 52.

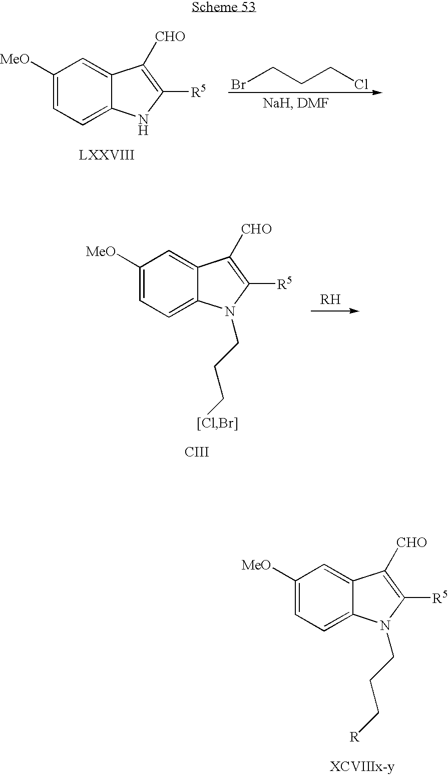

A dialkylation process was used to make the XCVIII compounds containing a heterocyclyl(propylene) substituent as R¹⁰, as shown in Scheme 53.

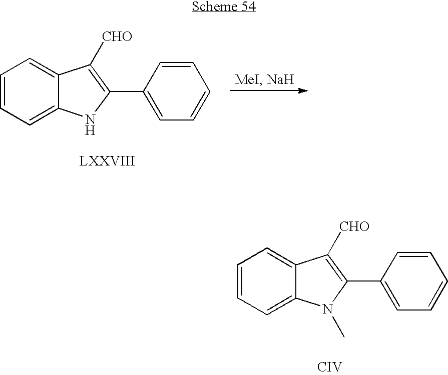

The preparation of 1-methyl-2-phenyl-1H-indole-3-carbaldehyde (CIV) is shown in Scheme 54.

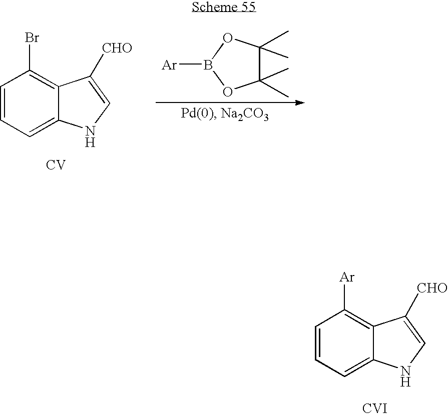

The preparation of 4-aryl-1H-indole-3-carbaldehyde (CVI) by Suzuki coupling is shown in Scheme 55.

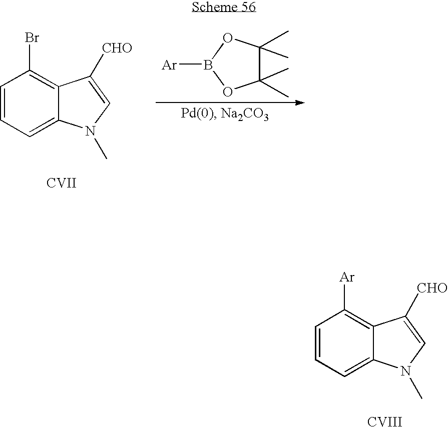

The preparation of 4-aryl-1-methyl-1H-indole-3-carbaldehyde (CVIII) by Suzuki coupling on the alkylated intermediate CVII is shown in Scheme 55.

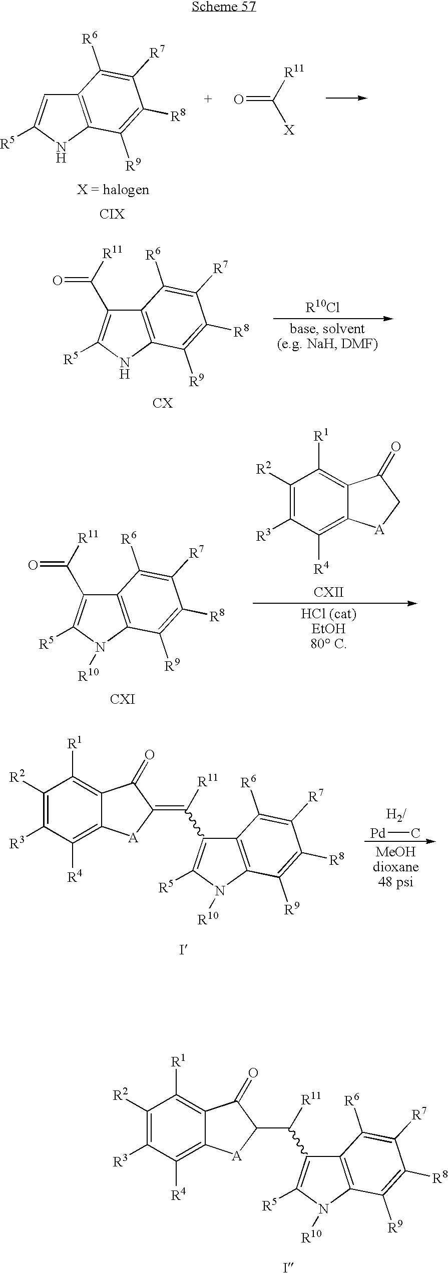

A synthesis of the 1H-indol-3-yl)methylene compounds of Formula I′ (compounds of Formula I with [symbol] a second carbon-to-carbon bond) and of the reduced indol-3-yl)methyl compounds I″ (compounds of Formula I with [symbol] absent) is shown in Scheme 57. Acylation with R¹¹C(O)X, wherein X is halogen, or Vilsmeier-Haack formylation, of a compound of formula CIX thereby producing a compound of formula CX and optionally alkylating the compound of formula CX with R¹⁰Cl, thereby producing a compound of Formula CXI.

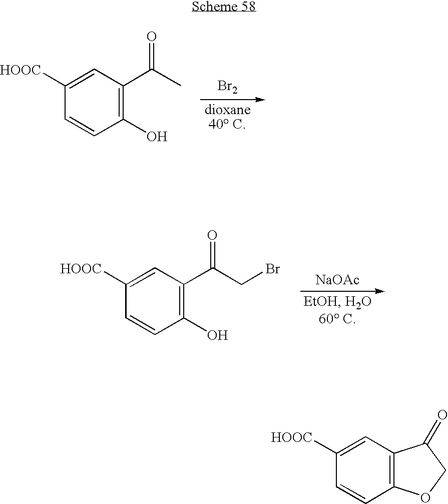

Preparation of 3-oxo-2,3-dihydrobenzofuran-5-carboxylic acid is shown above in Scheme 58 by a two-step bromination-cyclization process.

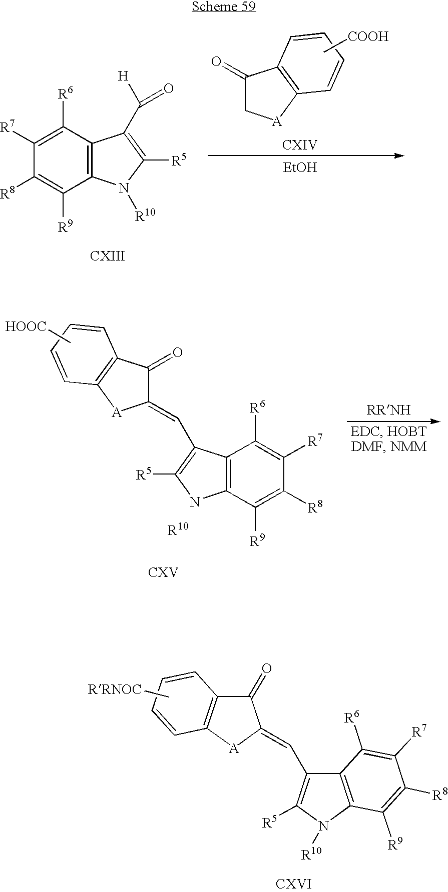

Condensation of 3-oxo-2,3-dihydrobenzofuran carboxylic acids CXIV with 1H-indole-3-carbaldehydes CXIII as shown above in Scheme 59.

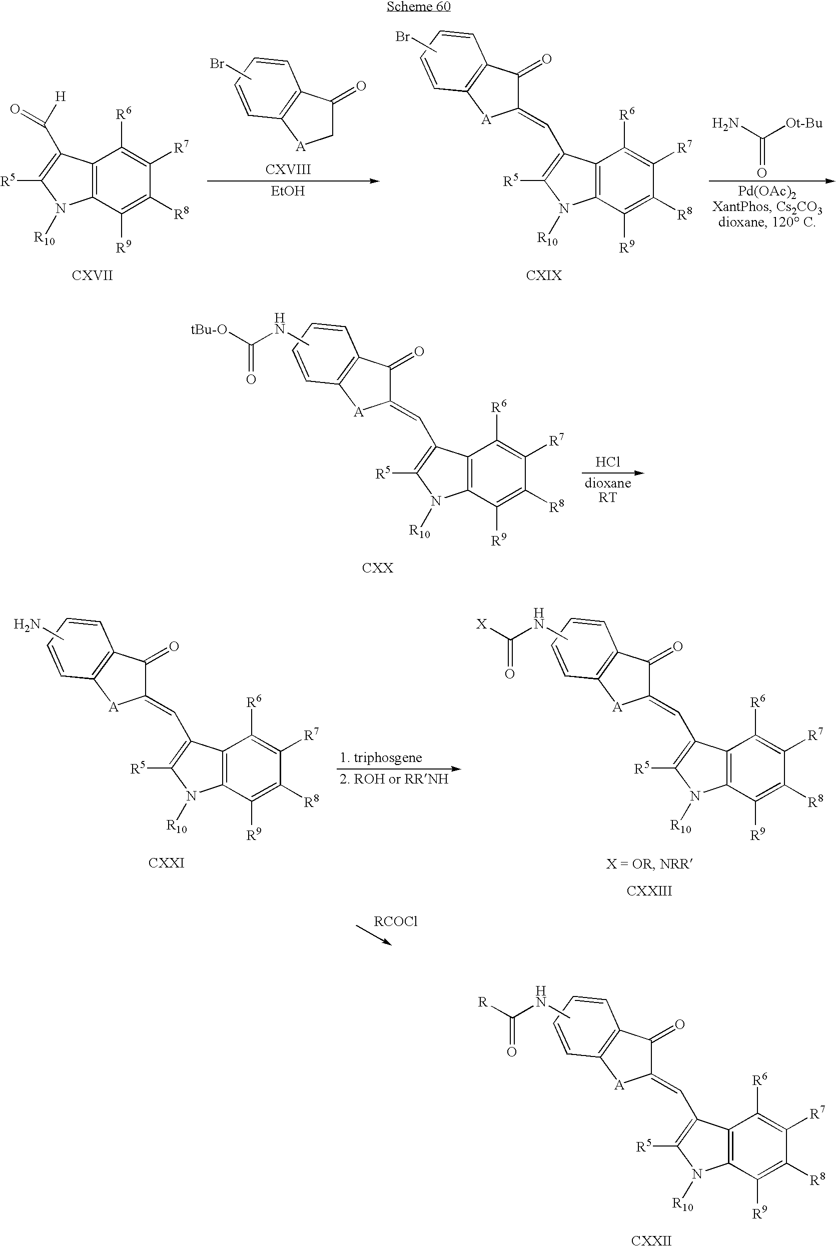

Condensation of bromo-3-oxo-2,3-dihydrobenzofuran CXVIII with 1H-indole-3-carbaldehydes CXVII as shown above in Scheme 60.

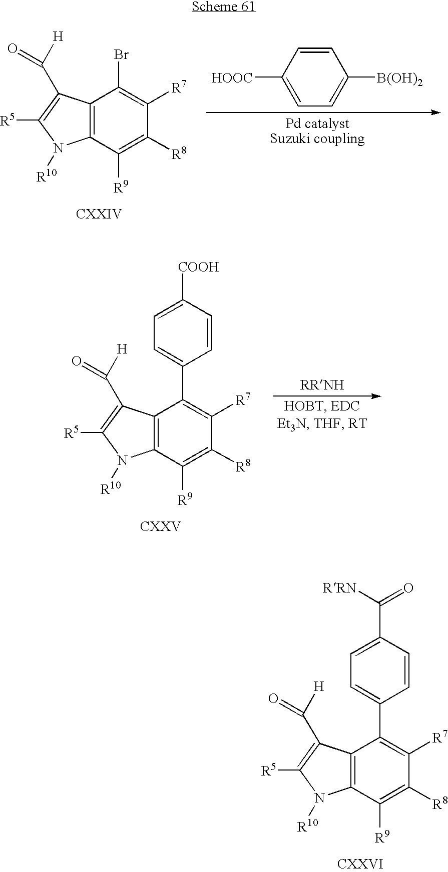

Preparation of 4-(3-formyl-1H-indol-4-yl)benzamide intermediates (CXXVI) as shown above in Scheme 61 by Suzuki coupling on the 4-bromo-3-formyl-1H-indol-4-yl)benzamide CXXV.

One of skill in the art will recognize that Schemes 1-61 can be adapted to produce the other compounds of Formula I and pharmaceutically acceptable salts of compounds of Formula I according to the present invention.

### EXAMPLES

The following abbreviations are used herein and have the indicated definitions: ACN is acetonitrile, AcOH is acetic acid, and ATP is adenosine triphosphate. Biotage Initiator™ 60 is a 60-position sample microwave synthesizer. Initiator™ is a registered trademark of Biotage AB, Uppsala, Sweden. BOC is t-butoxycarbonyl. Celite™ is flux-calcined diatomaceous earth. Celite™ is a registered trademark of World Minerals Inc. CHAPS is (3-[(3-cholamidopropyl)dimethylammonio]-1-propanesulfonic acid. The ISCO Companion™ is a personal flash chromatography system. Companion® is a registered trademark of Teledyne Isco Inc. (USA). DEAD is diethyl azodicarboxylate, DIAD is diisopropylazodicarboxylate, DMAP is dimethyl aminopyridine, DME is 1,2-dimethoxyethane, DMF is N,N-dimethylformamide, DMF-DMA is dimethylformamide dimethyl acetal, and DMSO is dimethylsulfoxide. DPBS is Dulbecco's Phosphate Buffered Saline Formulation. EDCI is 3′-dimethylaminopropyl)carbodiimide or water-soluble carbodiimide, EDTA is ethylenediaminetetraacetic acid, ESI stands for Electrospray Ionization, EtOAc is ethyl acetate, and EtOH is ethanol. HBTU is O-benzotriazole-N,N,N′,N′-tetramethyl-uronium-hexafluoro-phosphate, HEPES is 4-(2-hydroxyethyl)-1-piperazineethanesulfonic acid, GMF is glass microfiber, HOBT is N-hydroxybenzotriazole, Hunig's Base is diisopropylethylamine, HPLC is high-pressure liquid chromatography, LPS is lipopolysaccharide. MeCN is acetonitrile, MeOH is methanol, MS is mass spectrometry, and NEt₃ is triethylamine. Ni(Ra) is Raney™ nickel, a sponge-metal catalyst produced when a block of nickel-aluminum alloy is treated with concentrated sodium hydroxide. Raney™ is a registered trademark of W. R. Grace and Company. NMP is N-methylpyrrolidone, NMR is nuclear magnetic resonance, PBS is phosphate-buffered saline (pH 7.4), RPMI 1640 is a buffer (Sigma-Aldrich Corp., St. Louis, Mo., USA), SDS is dodecyl sulfate (sodium salt), SRB is Sulforhodamine B, TCA is trichloroacetic acid, TFA is trifluoroacetic acid, THF is tetrahydrofuran, THP is tetrahydro-2H-pyran-2-yl. TLC is thin-layer chromatography and TRIS is tris(hydroxymethyl)aminomethane.

### Synthetic Methods

The following methods outline the synthesis of the Examples of the present invention.

### I. Synthesis of Benzofuranone Intermediates

### Preparation of 4,6-dihydroxybenzofuranone (Compound A)

To a solution of phloroglucinol (2 g, 16 mmol, 1 eq.) in ethyl ether (20 mL), ClCH₂CN (10 mL), ZnCl₂ (0.2 g, 1.6 mmol, 0.1 eq.) and 10% HCl/Et₂O (15 mL) were added. The mixture was stirred at room temperature overnight. The yellow precipitate (imine hydrochloride) was filtered off and washed three times with ethyl ether. Then, it was dissolved in 25 mL of water and heated at 100° C. overnight. The red solid was filtered off, washed three times with water and dried to give pure 4,6-dihydroxy-benzofuran-3-one. Yield: 70%. MS (m/z): 167.2 (MH⁺).

### Preparation of 4-hydroxybenzofuranone (Compound B)

LiHMDS (1M solution in THF, 3.1 mL, 3.1 mmol, 3.6 eq.) was slowly added to a solution of 2′,6′-dihydroxyacetophenone (131 mg, 0.86 mmol, 1 eq.) in anhydrous THF (4.5 mL) under argon atmosphere at −78° C. After 30 minutes, TMSCl (0.65 mL, 5.16 mmol, 6 eq.) was added and the resulting mixture was stirred for 4 hours. Then NBS (171 mg, 0.95 mmol, 1.1 eq.) was slowly added and the solution was stirred for 1 hour at −78° C. and for 10 minutes at rt. 1M NaOH (2 mL) was added and the resulting solution was stirred until complete disappearance of the starting material. The reaction was quenched by adding 1M HCl until pH 4. The aqueous layer was extracted with EtOAc and the collected organic extracts were washed with brine, dried on anhydrous Na₂SO₄ and evaporated under reduced pressure. The oily crude mixture was purified by silica gel column chromatography (eluent: EtOAc/petroleum ether 15:85). The title compound was obtained as a pale yellow solid. Yield: 46%. MS (m/z): 151.5 (MH⁺).

### Preparation of monosubstituted 6-hydroxy benzofuranones (Compounds C-O)

### Preparation of 2-fluoro-3-methoxy-phenol

Hydrogen peroxide (35% in water, 5 mL) was added to a solution of 2-fluoro-3-methoxyphenylboronic acid (500 mg, 2.94 mmol) in dioxane (5 mL). The reaction mixture was stirred at 100° C. for 2.5 hours and then allowed to cool to rt. water was added and the aqueous layer was extracted with methylene chloride. The combined organic layers were dried on Na₂SO₄ and evaporated affording the title compound as dark oil. Yield: 71%. MS (m/z): 143.1 (MH⁺).

General Procedure for the Demethylation with BBr₃

To a solution of the methoxy-derivative (8.7 mmol) in methylene chloride (40 mL), cooled to −78° C., BBr₃ (1 M in methylene chloride, 4 eq. for each methoxy group) was added in drops. The reaction was stirred overnight allowing to the cooling bath to expire. The mixture was cooled again to −78° C. and quenched by addition of water in drops. The aqueous layer was extracted with EtOAc. The combined organic layers were dried on Na₂SO₄ and evaporated. The residue was triturated with EtOAc to give crude resorcinol that was used for the following reaction without further purification. This procedure was used to obtain the following compounds:
2-Fluoro-benzene-1,3-diol

Yield: 93%. MS (m/z): 129.1 (MH⁺).
5-Fluoro-benzene-1,3-diol

Yield: 97%. MS (m/z): 129.2 (MH⁺).
5-Chloro-benzene-1,3-diol

Yield: 87%. MS (m/z): 145.4 (MH⁺).

General Procedure for the Preparation of 6-hydroxybenzofuranones

Chloroacetyl chloride (0.33 mL, 4.15 mmol, 1.2 eq.) was added to a suspension of AlCl₃ (2.3 g, 17.3 mmol, 5 eq.) in nitrobenzene (6 mL), cooled to 0° C. The selected resorcinol (3.46 mmol, 1 eq.) was dissolved in nitrobenzene (6 mL) and added at 0° C. to the reaction mixture. The reaction was stirred at room temperature overnight, then poured into ice and extracted with EtOAc. The organic layer was extracted with 1 N NaOH; the separated aqueous layer was acidified with HCl and extracted with EtOAc. The combined organic layers were dried on Na₂SO₄ and evaporated. The crude mixture was triturated with Acute or methylene chloride to give pure benzofuranone compounds. This procedure was used to obtain the following compounds:

### 6-Hydroxy-4-methyl-benzofuran-3-one (C)

Yield: 17%. MS (m/z): 165.1 (MH⁺).

### 6-Hydroxy-5-methyl-benzofuran-3-one (D)

Yield: 69%. MS (m/z): 165.1 (MH⁺).

### 6-Hydroxy-7-methyl-benzofuran-3-one (E)

Yield: 22%. MS (m/z): 165.2 (MH⁺).

### 4-Fluoro-6-hydroxy-benzofuran-3-one (F)

Yield: 27%. MS (m/z): 169.1 (MH⁺)

### 5-Fluoro-6-hydroxy-benzofuran-3-one (G)

Yield: 28%. MS (m/z): 169.1 (MH⁺).

### 7-Fluoro-6-hydroxy-benzofuran-3-one (H)

Yield: 29%. MS (m/z): 169.2 (MH⁺).

### 4-Chloro-6-hydroxy-benzofuran-3-one (I)

Yield: 9%. MS (m/z): 185.1 (MH⁺).

### 5-Chloro-6-hydroxy-benzofuran-3-one (L)

Yield: 38%. MS (m/z): 185.1 (MH⁺).

### 7-Chloro-6-hydroxy-benzofuran-3-one (M)

Yield: 30%. MS (m/z): 185.3 (MH⁺).

### 5-Bromo-6-hydroxy-benzofuran-3-one (N)

Yield: 51%. MS (m/z): 228.9 (MH⁺).

### 4-Bromo-6-hydroxy-benzofuran-3-one (O)

Yield: 20%. MS (m/z): 229.0 (MH⁺).

### Preparation of 4,6-dimethoxybenzofuran-3(2H)-one (Compound P)

To a mixture of 3,5-dimethoxyphenol (47.1 g, 306 mmol), 2-chloroacetonitrile (23.07 g, 306 mmol) and zinc chloride (22.90 g, 168 mmol) in ether (450 mL) was bubbled thru Hydrochloric acid gas over 2 hours. An oil separates, this mixture was allowed to stir overnight. The ether was decanted from the now solidified oil, the solid rinsed with fresh ether, and the ether decanted. To the solid was added 400 mL of water and the mixture boiled for 1 hour, cooled to RT, filtered, washed with water. The solid was mixed with 50 grams of sodium acetate and 400 mL ethanol and the mixture heated at reflux for 5 hours and cooled. The solid was collected and washed with ethanol. The solid was washed with dichloromethane. The washes were evaporated and the solid isolated with ethyl acetate to give 4,6-dimethoxybenzofuran-3(2H)-one (7.85 g, 40.4 mmol, 13.23% yield).

### Preparation of 7-bromo-4-methoxybenzofuran-3(2H)-one (Compound Q)

To a solution of 1-(3-bromo-2-hydroxy-6-methoxyphenyl)ethanone (6.49 g, 26.5 mmol) in triethylamine (17 mL) and dichloromethane (120 mL) was added TBSCI (4.29 g, 28.5 mmol). This solution was stirred overnight. Reaction mixture was evaporated in-vacuo and treated with 150 mL water, stirred 1 hour, extracted with ether (3×75 mL). The combined ether extracts were combined, washed with 2N hydrochloric acid, water, dried over sodium sulfate, filtered, evaporated and the resulting semi-solid 1-[3-bromo-2-(tert-butyldimethylsilyloxy)-6-methoxyphenyl]ethanone (9.35 g, 26.0 mmol, 98% yield), used as is in the next step.

To a solution of 1-(3-bromo-2-(tert-butyldimethylsilyloxy)-6-methoxyphenyl)ethanone (9.35 g, 26.0 mmol) in TEA (17 mL) and dichloromethane (120 mL) was added TMSOTf (5.64 mL, 31.2 mmol), cooled with an ice bath. This solution was stirred overnight and allowed to warm to RT. Chloroform was added, 120 mL, and the mixture extracted with brine (2×150 mL). The organic layer was dried over sodium sulfate, filtered and evaporated to give a dark brown semi-solid, placed under high-vacuum to remove volatiles, 1-[3-bromo-2-(tert-butyldimethylsilyloxy)-6-methoxyphenyl]vinyloxytrimethylsilane (12.18 g, 26.0 mmol, 100% yield), assumed to be 92% pure, used as is for the next step.

To a solution of 1-[3-bromo-2-(tert-butyldimethylsilyloxy)-6-methoxyphenyl]vinyloxytrimethylsilane (12.18 g, 26.0 mmol) in carbon tetrachloride (120 mL), (some dark oil does not dissolve) cooled in an ice-bath, was added bromine (1.512 mL, 29.3 mmol) in 25 mL carbon tetrachloride in drops over 15 minutes. This was stirred at ice bath temp for 30 minutes then the ice bath was removed and the reaction allowed to warm to room temperature. Reaction mixture was treated with 200 mL water, layers separated. Aqueous extracted with concentrated hydrochloric acid (2×50 mL). Combined organic layers washed with aqueous Na₂S₂O₃, dried over sodium sulfate, filtered thru a little Magnesol™, evaporated to give an orange oil, 11.38 g, 2-bromo-1-[3-bromo-2-(tert-butyldimethylsilyloxy)-6-methoxyphenyl]ethanone, used as is in the next step.

To a solution of 2-bromo-1-[3-bromo-2-(tert-butyldimethylsilyloxy)-6-methoxyphenyl]ethanone (11.38 g, 26.0 mmol) in tetrahydrofuran (100 mL), cooled in an ice-bath, was added tetrabutylammonium fluoride (29 ml, 29.0 mmol) (1M in tetrahydrofuran). This was stirred at ice bath temp for 10 minutes then the ice bath was removed and the reaction allowed to warm to room temperature, stirred for 30 minutes. Reaction mixture was quenched with 30 mL saturated ammonium chloride solution. The tetrahydrofuran was removed in-vacuo; water and ether were added. The aqueous layer was extracted with ether (2×25 mL). Combined ether layers washed with water, brine, dried over sodium sulfate, filtered and evaporated to give a yellow residue, purified by chromatography using a hexane-ethyl acetate gradient the product peak was collected, evaporated and the solid isolated with 1:1 hexanes-ethyl acetate, washed with fresh solvent and dried to give a pale yellow solid, 7-bromo-4-methoxybenzofuran-3(2H)-one (587 mg, 9.30% yield).

### Preparation of 6-hydroxy-4-methoxybenzofuran-3(2H)-one (Compound R)

A mixture of 5-methoxybenzene-1,3-diol (10.05 g, 71.7 mmol), 2-chloroacetonitrile (5.41 g, 71.7 mmol), zinc chloride (5.38 g, 39.4 mmol) and ether (100 ml) was stirred in a 500 mL 3N Morton flask. Dry hydrogen chloride gas was bubbled through, solids dissolved and were replaced by a dark oil. After an hour of bubbling hydrochloric acid gas thru the mixture the oil became a salmon-colored solid. Hydrochloric acid gas is bubbled through for an additional hour. The mixture was stirred overnight. The mixture was filtered, and the flask rinsed with ether and this ether was used as a wash. Any solids remaining in the flask are left there. The solids were transferred back to the flask and treated with 100 mL of 2N hydrochloric acid and the mixture stirred and brought to reflux. All solids dissolved after heating for a while some solid precipitates. Heated for 2 hours and cooled, the salmon colored solid collected and washed well with water and dried, 9.73 g. A one gram portion of this was purified by chromatography using a hexane-ethyl acetate gradient; the product peak was collected, evaporated to give a yellow solid, 180 mg, MS (m/z) 181.2 (MH⁺), used as is for the next step.

### Preparation of 6-bromo-1-benzofuran-3(2H)-one (Compound S)

To a stirred solution of boron trichloride in methylene chloride (1.0 M, 6 mL, 6.0 mmol) at 0° C. was added a mixture of 3-bromophenol (870 mg, 5 mmol) in 2 mL of methylene chloride followed by chloroacetonitrile (0.38 mL, 6 mmol) and aluminum chloride (334 mg, 2.5 mmol). The mixture was stirred at room temperature for 20 hours. Then, ice and hydrochloric acid (2N, 4 mL, 8 mmol) were added and the mixture was stirred for 30 minutes. The mixture was extracted with methylene chloride (×3) and the organic layer was washed with saturated sodium chloride solution, dried over magnesium sulfate, and concentrated. The residue was purified by chromatography over silica, eluting with hexanes to 5% ethyl acetate in hexanes. The desired 1-(4-bromo-2-hydroxyphenyl)-2-chloroethanone was obtained as a mixture with the starting material 3-bromophenol, and was used without further purification. MS (m/z): 246.9 (MH−).

The crude product in the previous step was dissolved in 20 mL of acetonitrile and 3 mL of triethylamine was added. The mixture was stirred at room temperature for 40 minutes, and concentrated. The residue was purified by chromatography over silica, eluting with hexanes to 2% ethyl acetate in hexanes. The desired 6-bromo-1-benzofuran-3(2H)-one was obtained as a yellow solid (350 mg). MS (m/z): 213.0 (MH⁺).

### II. Synthesis of Indole-3-carbaldehyde Intermediates

### Preparation of 5-methoxy-2-phenyl-1H-indole-3-carbaldehyde

POCl₃ (2.05 mL, 22 mmol, 1.1 eq) was added to DMF (7.74 mL, 5 eq) at 0° C. Let stir 30 minutes. The Vilsmeier-Haack reagent was added to a stirring solution of 2-phenyl-5-methoxyindole (4.47 g, 20 mmol, 1 eq) in DMF (15 mL) at 5° C. Stirred in ice water bath 30 minutes, then let reaction warm to ambient temperature. The reaction was poured onto ice and basified to pH 10 with 5N aqueous NaOH solution. The reaction was heated to boiling then allowed to cool and acidified to pH 4 with 2N aqueous HCl solution. The resulting precipitate was filtered to isolate title compound as a solid dried in vacuo.

### Preparation of 5-Methoxy-2-methyl-1-(2-(4-methylpiperazin-1-yl)ethyl)-1H-indole-3-carbaldehyde

Step 1)

### 5-methoxy-2-methyl-1H-indole-3-carbaldehyde

POCl₃ (2.05 mL, 22 mmol, 1.1 eq) was added to DMF (7.74 mL, 5 eq) at 0° C. Let stir 30 minutes. The Vilsmeier-Haack reagent was added to a stirring solution of 2-methyl-5-methoxyindole (3.22 g, 20 mmol, 1 eq) in DMF (15 mL) at 5° C. Stirred on ice water bath 30 minutes, then let reaction warm to ambient temperature. The reaction was poured onto ice and Basified to pH 10 with 5N aqueous NaOH solution. The mixture was heated to boiling and the allowed to cool. The mixture was acidified to pH 4 with 2N aqueous HCl solution and the resulting precipitate formed filtered to isolate the title compound as a solid.

Step 2

### 1-(2-chloroethyl)-5-methoxy-2-methyl-1H-indole-3-carbaldehyde

To 5-methoxy-2-methyl-1H-indole-3-carbaldehyde (1.0 g, 5.7 mmol) in DMF (100 mL) cooled to 0 C was added NaH (0.46 g of 60% dispersion in mineral oil, 11.4 mmol, 2 eq.). The resulting suspension was stirred for 15 minutes followed by addition of 1-bromo-2-chloro-ethane (2.4 mL, 29 mmol, 5 eq.). The ice was removed and the mixture stirred overnight at room temperature. The reaction was quenched with the addition of water (50 mL), extracted with EtOAc (100 mL), washed with water (50 mL) and brine (50 mL) and dried (Na2SO4) and concentrated in vacuo. Silica gel chromatography (5:5 Hex:EtOAc) afforded 0.28 g of the title compound as a white solid.

Step 3

### 5-Methoxy-2-methyl-1-(2-(4-methylpiperazin-1-yl)ethyl)-1H-indole-3-carbaldehyde

To 1-(2-chloroethyl)-5-methoxy-2-methyl-1H-indole-3-carbaldehyde (60 mg, 0.24 mmol) in acetonitrile (5 mL) was added K₂CO₃ (165 mg, 1.2 mmol, 5 eq.), KI (99 mg, 0.6 mmol, 2.5 eq.), and N-Methyl piperazine (86 μL, 0.95 mmol, 4 eq.). The resulting suspension was heated to 90 C and stirred for 48 hrs. To the reaction mixture was added water (10 mL) and EtOAc (10 mL). The layers were separate and the aqueous layer washed with EtOAc (20 mL). Combination of the organic layers followed by drying (Na₂SO₄) and concentration in vacuo afforded the crude product used directly in the next reaction.

### Preparation of 4-bromo-1-methyl-H-indole-3-carbaldehyde)

A mixture of 3 g (13.38 mmol) of 4-bromo-3-formylindole, and 482.9 mg (20.1 mmol) of sodium hydride was stirred in N,N-dimethylformamide (30 mL) at 0° C. until no more gas evolved. Then 1.25 mL (20.1 mmol) of methyl iodide was added into the mixture, and let it warm up to room temperature overnight. To the mixture was added a solution of ethyl acetate and ether (1:1). The organic layer was washed five times with brine, dried over sodium sulfate, and evaporated to give a pink solid 2.8 g (88% yield). MS (m/z) 238.1 (MH⁺).

### Preparation of 4-(4-isopropoxy-phenyl)-1-methyl-1H-indole-3-carboxylaldehyde

A mixture of 300 mg (1.26 mmol) of 4-bromo-1-methyl-H-indole-3-carbaldehyde, 340.2 mg (1.89 mmol) of isopropoxyphenylboronic acid, 145.6 mg (0.126 mmol) of tetrakis(triphenylphosphine)palladium(0), and saturated aqueous sodium carbonate (1 mL), was placed in a microwave vial. To the mixture was added 3 mL of 1,2-dimethoxyethane. The sealed tube was heated by microwave for twenty minutes at 120° C. After cooling, the mixture was filtered through Celite™ and washed with ethyl acetate. After the solvent was evaporated, the residue was purified by column chromatography (70% ethyl acetate in hexane) to give 283 mg of 4-(4-isopropoxy-phenyl)-1-methyl-1H-indole-3-carboxylaldehyde as a light brown solid (77% yield). MS (m/z) 294.4 (MH⁺).

### Preparation of 4-bromo-1-(2-chloroethyl)-1H-indole-3-carbaldehyde

A mixture of 5 g (22.23 mmol) of 4-bromo-3-formylindole (Frontier), and 1.6 g (66.69 mmol) of sodium hydride was stirred in N,N-dimethylformamide (60 mL) at 0° C. until no more gas evolved. Then, 4.1 mL (44.46 mmol) of 1-chloro-2-iodoethane was added into the mixture, and let it warm up to room temperature overnight. To the mixture was added a solution of ethyl acetate. The organic layer was washed five times with brine, dried over sodium sulfate and evaporated to give a off white solid. The solid was purified by column chromatography to give 2.4 g of 4-bromo-1-(2-chloroethyl)-1H-indole-3-carbaldehyde (38% yield). MS (m/z) 287.55 (MH⁺).

### Preparation of 4-bromo-1-[2-(4-methylpiperizin-1-yl)ethyl]-1H-indole-3-carbaldehyde

A mixture of 2 g (7.0 mmol) of 4-bromo-1-(2-chloroethyl)-1H-indole-3-carbaldehyde, 3.1 mL (28 mmol) of 1-methylpiperazin, 2.1 g (14.0 mmol) of sodium iodide and 2.39 g (7.0 mmol) of tetrabutylammonium iodide was stirred in 20 mL of 1-methylpyrrolidinone at 80° C. for two hours. After cooling the mixture to room temperature, 30 mL of water was added and made basic with saturated potassium carbonate. The solution was extracted three times with methylene chloride, dried over sodium sulfate, and evaporated. The product was purified by column chromatography (20% methanol:methylene chloride) to give 1.6 g of 4-bromo-1-[2-(4-methylpiperizin-1-yl)ethyl]-1H-indole-3-carbaldehyde as a yellow oil (67% yield). MS (m/z) 351.25 (MH⁺).

### Preparation of 3-formyl-1-methyl-2-phenyl-1H-indole-4-carbonitrile

Step 1

4-Cyanoindole (5.0 g, 35.2 mmol) was dissolved in 70 mL DMF and cooled to 0° C. 60% sodium hydride (2.1 g, 52.8 mmol) was added in portions and let react for 30 minutes. Iodomethane (4.4 mL, 70.4 mmol) was added and let warm to room temperature. The reaction was then quenched with cold water and extracted with ethyl acetate 3 times. The organics were washed with brine, dried over magnesium sulfate, and concentrated in vacuo. The residue was filtered and dried to afford 1-methyl-1H-indole-4-carbonitrile (5.2 g, 33.3 mmol, 95% yield).

Step 2

In a 25 mL round bottom flask was combined 1-methyl-1H-indole-4-carbonitrile (0.41 g, 2.6 mmol), triphenylphosphine (14 mg, 0.052 mmol), palladium II acetate (30 mg, 0.13 mmol), cesium acetate (1.04 g, 5.2 mmol), iodobenzene (0.35 mL, 3.12 mmol) in 1.5 mL N,N-dimethylacetamide. The reaction mixture was heated to 125° C. for 24 hours. The black mixture was diluted with dichloromethane, filtered through Celite, concentrated and purified on a 40 g ISCO silica column using 20% ethyl acetate:hexane gradient. Combined desired fractions, concentrated in vacuo to afford 0.21 g (0.90 mmol, 35% yield) of 1-methyl-2-phenyl-1H-indole-4-carbonitrile. MS (m/z) 233.4 (MH⁺).

Step 3

In an oven-dried 3 neck round bottom flask equipped with N₂ and thermocouple was charged DMF (0.31 mL, 3.96 mmol) and was cooled to 0° C. POCl₃ (0.092 mL, 0.99 mmol) was added by drops, while keeping the temperature before 5° C. 1-Methyl-2-phenyl-1H-indole-4-carbonitrile (0.21 g, 0.9 mmol) was dissolved in 3 mL DMF and added by drops to the reaction mixture. This was heated to 35 C for 2 hours. The reaction was cooled to room temp, then quenched with ice. Solids formed which were filtered and dried in vacuo to afford 0.153 g (0.588 mmol, 66% yield) of 3-formyl-1-methyl-2-phenyl-1H-indole-4-carbonitrile. MS (ESI): MS (m/z) 261.3 (MH⁺).

### Synthesis of 5-methoxy-indole-3-carbaldehydes

### Preparation of 5-methoxy-indole-3-carbaldehyde, 5-methoxy-2-methyl-indole-3-carbaldehyde, and 3-formyl-5-methoxy-indole-2-carboxylic acid

POCl₃ (1.6 mL, 17 mmol, 1.1 eq.) was added to DMF (6 mL) at 0° C. and the solution was stirred for 30 minutes. This mixture was added to a stirring solution of the selected 5-methoxy-indole (15.5 mmol, 1 eq.) in DMF (11.5 mL) at 0° C. The resulting mixture was stirred at 0° C. for 30 minutes, then allowed to warm to room temperature. The reaction was poured into ice, basified to pH 10 with 5 N NaOH, warmed to room temperature, heated at reflux for 5 minutes and allowed to cool to rt. Finally, it was acidified to pH 4 with 2 N HCl and the resulting precipitate was filtered and washed with water until pH 7. The solid product was dried under vacuum.

### 5-Methoxy-indole-3-carbaldehyde

Yield: 85%. MS (m/z): 176.2 (MH⁺).

### 5-Methoxy-2-methyl-indole-3-carbaldehyde

Yield: 94%. MS (m/z): 190.2 (MH⁺).

### 3-Formyl-5-methoxy-indole-2-carboxylic acid

Yield: 98%. MS (m/z): 220.3 (MH⁺).

### Preparation of 3-formyl-5-methoxy-indole-2-carboxylic acid dimethylamide

CDI (0.55 g, 3.4 mmol, 1.3 eq.) was added to a solution of 5-methoxy-indole-2-carboxylic acid (0.5 g, 2.6 mmol, 1.0 eq.) in methylene chloride (10 mL) at 0° C. The reaction mixture was stirred for 30 minutes, and then dimethylamine (3 mL of 28% solution in THF, 10 eq.) was added. The reaction mixture was stirred at room temperature in a sealed tube overnight, and then water was added. The aqueous layer was separated and extracted with methylene chloride. The combined organic layers were washed with saturated NaHCO₃ and brine, dried on Na₂SO₄, and evaporated to give 5-methoxy-indole-2-carboxylic acid dimethylamide. Yield: 75%. MS (m/z): 219.3 (MH⁺).

Phosphorus tribromide (155 mg, 0.57 mmol, 2.5 eq.) was added by drops to a solution of dry DMF (39 mg, 0.68 mmol, 3 eq.) in dry methylene chloride (1 mL) at 0° C. The mixture was stirred at 0° C. for 1 hour and a pale yellow suspension formed. A solution of 5-methoxy-indole-2-carboxylic acid dimethylamide (50 mg, 0.23 mmol) in dry methylene chloride (1 mL) was added and the resulting mixture was heated at reflux for 3 hours. The reaction mixture was poured into ice and neutralized with NaHCO₃. The aqueous layer was separated and extracted with methylene chloride. The combined organic layers were dried on Na₂SO₄. Evaporation of the solvent afforded the crude product that was purified by silica gel column chromatography (eluent: CHCl₃/MeOH 98:2). Yield: 44%. MS (m/z): 247.3 (MH⁺).

### Preparation of 5-methoxy-2-cyclopropyl-indole-3-carbaldehyde

A solution of 4-methoxy-2-methylaniline (10 g, 72.9 mmol, 1 eq.) and tert-butyl dicarbonate (18.3 g, 84.8 mmol, 1.2 eq.) in THF (90 mL) was heated at reflux for 2 hours. After cooling, the reaction mixture was evaporated under reduced pressure and the residue was dissolved in EtOAc. The organic layer was washed with a saturated NH₄Cl and brine, dried on Na₂SO₄, and evaporated to give crude N-(tert-butoxycarbonyl)-4-methoxy-2-methylaniline that was used without further purification. Yield: quant. MS (m/z): 238.9 (MH⁺).

Et₃N (3.3 mL) was added to a solution of MeNH(OMe)-HCl (1.2 g, 12.4 mmol, 1 eq.) in methylene chloride (35 mL). The solution was stirred at room temperature for 30 minutes, then the reaction was cooled to 0° C. and cyclopropanecarbonyl chloride (1 g, 12.4 mmol, 1 eq.) was added. After 5 hours, the reaction mixture was diluted with methylene chloride, washed with 1 N HCl and saturated NaHCO₃. The organic layer was dried on Na₂SO₄ and evaporated to give crude N-methoxy-N-methylcyclopropanecarboxamide, which was utilized in the next step without further purification. Yield: 94%.

A solution of N-(tert-Butoxycarbonyl)-4-methoxy-2-methylaniline (2.7 g, 11.6 mmol) in THF (34 mL) was cooled to −78° C. under N₂ and sec-BuLi (1.3 M in cyclohexane, 17.9 mL, 23.2 mmol) was added slowly keeping the temperature below −40° C. After 15 minutes, a solution of N-methoxy-N-methylcyclopropanecarboxamide (1.5 g, 11.6 mmol) in THF (34 mL), was added by drops. The reaction mixture was stirred for 1 hour, then the cooling bath was removed and the mixture was stirred for additional 1 hour. The reaction was poured into a mixture of Et₂O and 1 N HCl. The organic layer was separated, washed with water, dried on Na₂SO₄, and evaporated under reduced pressure to give crude t-butyl-2-(2-cyclopropyl-2-oxoethyl)-4-methoxyphenyl carbamate. The desired compound was purified by flash chromatography. Yield: 61%. MS (m/z): 306.3 (MH⁺).

A solution of t-butyl-2-(cyclopropyl-2-oxopropyl)-4-methoxyphenylcarbamate (1.5 g, 4.9 mmol) and trifluoroacetic acid (5 mL) in methylene chloride (25 mL) was stirred for 4 hours. Water was added and the organic layer separated, dried on Na₂SO₄ and evaporated to give 5-methoxy-2-cyclopropyl-indole. Yield: 69%.

For the formylation step, the same procedure described for 5-methoxy-indole-3-carbaldehyde and 5-methoxy-2-methyl-indole-3-carbaldehyde was used. Yield: 95%. MS (m/z): 216.2 (MH⁺).

### Preparation of 5-methoxy-2-trifluoromethyl-indole-3-carbaldehyde

A solution of N-(tert-butoxycarbonyl)-4-methoxy-2-methylaniline (2.6 g, 11 mmol) in THF (34 mL) was cooled to −78° C. and sec-BuLi (1.4 M in cyclohexane, 17.1 mL, 24 mmol, 2.2 eq.) was slowly added, keeping the temperature below −40° C. After 15 minutes, a solution of ethyl trifluoroacetate (1.56 mL, 13.1 mmol, 1.2 eq) in THF (34 mL) was by drops added. The cooling bath was removed and the mixture was stirred for 3 hours. The reaction was poured into a mixture of Et₂O and 1 N HCl. The organic layer was separated, washed with water, dried on Na₂SO₄, and evaporated under reduced pressure to give crude tert-butyl 2-(3,3,3-trifluoro-2-oxopropyl)-4-methoxyphenylcarbamate that was used in the following step without further purification. Yield: 92%.

A solution of tert-butyl 2-(3,3,3-trifluoro-2-oxopropyl)-4-methoxyphenylcarbamate (1.34 g, 4.9 mmol) and trifluoroacetic acid (5 mL) in methylene chloride (25 mL) was stirred for 24 hours. Water was added and the organic layer was separated, dried on Na₂SO₄, and evaporated to give 2-trifluoromethyl-5-methoxy-indole. Yield: 70%.

For the formylation step, the classical Vilsmeier-Haack procedure with POCl₃ was used performing the reaction at 50° C. A mixture of indole-3-carboxaldehyde and indole-4-carboxaldehyde formed. The title compound was isolated by trituration with Et₂O. Both the isomers were characterized:

### 2-(Trifluoromethyl)-5-methoxy-indole-3-carbaldehyde

MS (m/z): 244.3 (MH⁺).

### 2-(Trifluoromethyl)-5-methoxy-indole-4-carbaldehyde

MS (m/z): 244.3 (MH⁺).

### Preparation of 5-methoxy-2-(1-methyl-1H-pyrazol-4-yl)-indole-3-carbaldehyde

5-Methoxy isatin (0.2 g, 1.1 mmol, 1 eq.) was dissolved in hydrazine hydrate (1.2 mL, 38 mmol, 34 eq.) and heated at reflux for 15 minutes. The reaction mixture was poured into cold water and extracted with EtOAc. The combined organic extracts were dried on Na₂SO₄. The solvent was evaporated to afford crude 5-methoxy-1,3-dihydro-indol-2-one that was purified by silica gel column chromatography (eluent: hexane/EtOAc from 10:0 to 6:4). Yield: 27%. MS (m/z): 164.2 (MH⁺).

Phosphorous oxybromide (0.35 mL, 3.1 mmol, 2.5 eq.) was added drop wise to a solution of DMF (0.3 mL, 3.7 mmol, 3 eq.) in dry methylene chloride at 0° C. The mixture was stirred at 0° C. for 30 minutes, then a solution of 5-methoxy-1,3-dihydro-indol-2-one (0.2 g, 1.2 mmol, 1 eq.) in dry methylene chloride (2 mL) was added and the mixture was heated at reflux for 3 hours. The solution was neutralized with solid NaHCO₃ and extracted with methylene chloride. The organic layer was dried on Na₂SO₄ and evaporated under reduced pressure. The crude mixture was purified by silica gel column chromatography (eluent: hexane/EtOAc 6:4 to 4:6) to give pure 2-bromo-5-methoxy-indole-3-carbaldehyde. Yield: 45%. MS (m/z): 254.1 (MH⁺).

A stirred solution of 2-bromo-5-methoxy-indole-3-carbaldehyde (2.0 g, 7.9 mmol, 1 eq.) in DME (2 mL) was deoxygenated by bubbling argon for 10 minutes at rt. Pd(PPh₃)₄ (0.9 g, 0.8 mmol, 0.1 eq.) was added followed by a solution of 1-methyl-4-(4,4,5,5-tetramethyl-[1,3,2]dioxaborolan-2-yl)-1H-pyrazole (2.4 g, 11.63 mmol, 1.48 eq.) in ethanol (2.5 mL). 2M Na₂CO₃ (33 mL, 8.5 eq.) was also deoxygenated with argon and added. The resulting mixture was heated at 78° C. for 18 hours. The reaction mixture was cooled to room temperature, quenched with water and extracted with methylene chloride. Organic layer was dried on anhydrous Na₂SO₄ and evaporated under reduced pressure to give the crude product 1f. Yield: 89%. MS (m/z): 256.1 (MH⁺).

### 2-(3,5-Dimethyl-isoxazol-4-yl)-5-methoxy-indole-3-carbaldehyde

The compound was obtained with the same Suzuki coupling described with 1f, (3,5-dimethyl-4-(4,4,5,5-tetramethyl-[1,3,2]dioxaborolan-2-yl)-isoxazole was used as boronic reagent). The crude product was purified by silica gel column chromatography (eluent: EtOAc/hexane 1:1). Yield: 57%. MS (m/z): 271.3 (MH⁺).

### Preparation of 5-methoxy-2-pyrimidin-5-yl-indole-3-carbaldehyde

To a stirred solution of Pd(PPh₃)₄ (0.818 g, 0.7 mmol, 0.1 eq.) in propanol (5 mL), deoxygenated 2M Na₂CO₃ (4.2 mL, 8.5 mmol, 1.2 eq.) was added and the resulting mixture was stirred for 10 minutes at room temperature under argon atmosphere. 2-Bromo-5-methoxy-indole-3-carbaldehyde (1.80 g, 7.08 mmol, 1 eq.) and 5-pyrimidinyl boronic acid (1.05 g, 8.5 mmol, 1.2 eq.) in 1-propanol (20 mL) were added and the reaction mixture was stirred for 10 minutes. The temperature was slowly raised to 80° C. and the reaction was stirred overnight. The reaction mass was cooled to room temperature, quenched with water and extracted with EtOAc. The organic layer was washed with 5% NaHCO₃ solution, brine, and dried on anhydrous Na₂SO₄. Evaporation of the solvent afforded a crude mixture that was purified by silica gel column chromatography (eluent: CHCl₃/MeOH 100:0 to 95:5). Yield: 50%. MS (m/z): 254.1 (MH⁺).

### Preparation of 5-methoxy-2-phenyl-indole-3-carbaldehyde

A solution of p-anisidine (3 g, 24 mmol, 1 eq.) and 2-bromoacetophenone (4.8 g, 24 mmol, 1 eq.) in DMA (5 mL) was heated at 170° C. with microwave irradiation for 1 hour. The reaction mixture was diluted with methylene chloride and washed with 2 N HCl. The organic layer was dried on Na₂SO₄ and evaporated. The crude mixture was filtered on a pad of silica gel (methylene chloride as eluent) and the obtained product was triturated with Et₂O. 5-Methoxy-2-phenylindole was obtained as a white solid. Yield: 40%. MS (m/z): 224.3 (MH⁺). For the formylation step, the same procedure described for 5-methoxy-indole-3-carbaldehyde and 5-methoxy-2-methyl-indole-3-carbaldehyde was used.

### Preparation of 5-methoxy-2-(4-methyl-piperazine-1-carbonyl)-indole-3-carbaldehyde

To a stirred solution of 5-methoxy-indole-2-carboxylic acid (0.3 g, 1.56 mmol, 1.0 eq.) in methylene chloride (10 mL) at 0° C., EDCI (0.36 g, 1.88 mmol, 1.2 eq.) and HOBT (0.23 g, 1.72 mmol, 1.1 eq.) were added. The mixture was stirred for 30 minutes, then N-methyl-piperazine (0.18 g, 1.88 mmol, 1.2 eq.) was added. The reaction was stirred at room temperature overnight, water was added, and organic layer was separated. The organic layer was washed with saturated NaHCO₃ and brine, dried on Na₂SO₄, and evaporated to give (5-methoxy-indol-2-yl)-(4-methyl-piperazin-1-yl)-methanone. Yield: 70%. MS (m/z): 274.4 (MH⁺). Classical Vilsmeier-Haack conditions were used on (5-methoxy-indol-2-yl)-(4-methyl-piperazin-1-yl)-methanone. Yield: 63%. MS (m/z): 302.2 (MH⁺).

### Preparation of 5-methoxy-2-(4-methyl-piperazin-1-ylmethyl)-indole-3-carbaldehyde

To a suspension of LiAlH₄ (0.15 g, 3.7 mmol, 3.7 eq.) in THF (10 mL), (5-methoxy-indol-2-yl)-(4-methyl-piperazin-1-yl)-methanone (0.50 g, 1.0 mmol) was added at 5° C. The resulting mixture was stirred for 3 hours, then it was quenched with saturated ammonium chloride solution and filtered. The filtrate was extracted with EtOAc. The organic layer was dried on Na₂SO₄ and evaporated. The crude product was purified by silica gel column chromatography (eluent: CHCl₃/MeOH 98:2). Yield: 85%. MS (m/z): 260.1 (MH⁺).

A solution of POCl₃ (1.18 g, 7.7 mmol, 5 eq.) in DMF (0.56 g, 7.7 mmol, 5 eq.) was stirred for 30 minutes at 0° C. 5-methoxy-2-(4-methyl-piperazin-1-ylmethyl)-indole (0.40 g, 1.5 mmol, 1 eq.) was added at 0° C. and the resulting mixture was stirred for 6 hours at room temperature. The reaction was quenched with ice, basified with NaOH to pH 9, and extracted with methylene chloride. The organic layer was dried on Na₂SO₄ and evaporated to give crude 5-methoxy-2-(4-methyl-piperazin-1-ylmethyl)-indole-3-carbaldehyde 1k. Yield: 95%. MS (m/z): 288.2 (MH⁺).

### Preparation of 2-dimethylaminomethyl-5-methoxy-indole-3-carbaldehyde

To a suspension of LiAlH₄ (1.03 g, 27.4 mmol, 10 eq.) in THF (20 mL), 5-methoxy-indole-2-carboxylic acid dimethylamide (0.60 g, 2.7 mmol, 1 eq.) was added at room temperature. The resulting mixture was stirred for 1 hour, then it was quenched with saturated ammonium chloride solution and filtered. The filtrate was extracted with EtOAc. The organic layer was dried on Na₂SO₄ and evaporated to give (5-methoxy-indol-2-ylmethyl)-dimethyl-amine. Yield: 90%. MS (m/z): 205.2 (MH⁺).

A solution of POCl₃ (0.93 g, 5.9 mmol, 5.9 eq.) in DMF (0.28 g, 4.9 mmol, 4.9 eq.) was stirred for 30 minutes at 0° C. To this solution, (5-methoxy-indol-2-ylmethyl)-dimethyl-amine (0.20 g, 1.0 mmol, 1 eq.) was added at 0° C. and the resulting mixture was stirred at room temperature overnight. The reaction was quenched with ice, basified with NaOH to pH 9, and extracted with methylene chloride. The organic layer was dried on Na₂SO₄ and evaporated to give the crude product that was purified by silica gel column chromatography (eluent: CHCl₃/MeOH 98:2). Yield: 95%. MS (m/z): 233.1 (MH⁺).

### Preparation of 5-methoxy-2-(morpholine-4-carbonyl)-indole-3-carbaldehyde

5-Methoxy-2-(morpholine-1-carbonyl)-indole-3-carbaldehyde is synthesized analogously to 1j, using morpholine instead of 1-methylpiperazine. Yield: 76%. MS (m/z): 289.1 (MH⁺).

### Preparation of 5-methoxy-2-(pyrrolidine-1-carbonyl)-indole-3-carbaldehyde

5-Methoxy-2-(pyrrolidine-1-carbonyl)-indole-3-carbaldehyde is synthesized analogously to 1j, using pyrrolidine instead of 1-methylpiperazine. Yield: 74%. MS (m/z): 273.1 (MH⁺).

### Preparation of 2-cyclopentyl-5-methoxy-indole-3-carbaldehyde

2-Cyclopentyl-5-methoxy-indole-3-carbaldehyde is synthesized analogously to 1d, using of cyclopentanecarbonyl chloride instead of cyclopropanecarbonyl chloride. Yield: 87%. MS (m/z): 244.3 (MH⁺).

### Preparation of 2-cyclohexyl-5-methoxy-indole-3-carbaldehyde

2-Cyclohexyl-5-methoxy-indole-3-carbaldehyde is synthesized analogously to 1d, using of cyclohexanecarbonyl chloride instead of cyclopropanecarbonyl chloride. Yield: 93%. MS (m/z): 258.3 (MH⁺).

### Preparation of 2-cyclobutyl-5-methoxy-indole-3-carbaldehyde

2-Cyclobutyl-5-methoxy-indole-3-carbaldehyde is synthesized analogously to 1d, using of cyclobutanecarbonyl chloride instead of cyclopropanecarbonyl chloride. Yield: 67%. MS (m/z): 230.3 (MH⁺).

### Synthesis of N-substituted 5-methoxy-indole-3-carbaldehydes

### For the preparation of 5-methoxy-indole-3-carbaldehydes, four general routes (A-D) were used

General procedure for the alkylation with 1-(2-chloro-ethyl)-imidazole (compounds with y=2)

To a solution of the selected 5-methoxy-indole-3-carbaldehyde 1× (5.7 mmol, 1 eq.) in acetonitrile (20 mL), K₂CO₃ (3.9 g, 28.5 mmol, 5 eq.), KI (2.3 g, 14 mmol, 2.5 eq.) and 1-(2-chloro-ethyl)-imidazole (3.0 g, 22.8 mmol, 4 eq.) were added. The resulting suspension was stirred at 90° C. for 24 hours, and then water was added. The aqueous layer was separated and extracted with EtOAc. The combined organic layers were dried on Na₂SO₄ and evaporated. The crude products were further purified as described below. According to this procedure, the following compounds were obtained.

### 1-(2-Imidazol-1-yl-ethyl)-5-methoxy-indole-3-carbaldehyde

Purified by silica gel column chromatography (eluent: CHCl₃/MeOH 95:5). Yield: 40%. MS (m/z): 270.3 (MH⁺).

### 3-Formyl-1-(2-imidazol-1-yl-ethyl)-5-methoxy-1H-indole-2-carboxylic acid dimethylamide

Purified by silica gel column chromatography (eluent: CHCl₃/MeOH 97:3). Yield: 72%. MS (m/z): 341.2 (MH⁺).

### General Procedure for the alkylation with 2-chloro-N,N-dimethyl-acetamide (compounds with y=5)

60% NaH in mineral oil (2.0 g, 50 mmol, 2.2 eq.) was pre-washed with hexane and suspended in dry DMF (4 mL) under nitrogen. The suspension was cooled with an ice bath and a solution of the selected 5-methoxy-indole-3-carbaldehyde 1× (22 mmol, 1 eq.) in dry DMF (8 mL) was added by drops over 15 minutes. The cooling bath was removed and the mixture was stirred for 30 minutes. The reaction mixture was cooled again and a solution of 2-chloro-N,N-dimethyl-acetamide (5.9 g, 44 mmol, 2 eq.) in dry DMF (8 mL) was added by drops over 10 minutes. The reaction mixture was stirred according to the conditions indicated below. The solvent was evaporated and the residue was partitioned between EtOAc and water. The combined organic layers were washed with water and brine and dried on Na₂SO₄. Evaporation of the solvent afforded a crude mixture that was purified by silica gel column chromatography. According to this procedure, the following compounds were obtained.

### 2-(3-Formyl-5-methoxy-indol-1-yl)-N,N-dimethyl-acetamide

Reaction conditions: room temperature for 18 hours. Purified by silica gel column chromatography (eluent: gradient from CHCl₃ to CHCl₃/MeOH 95:5). Yield: 44%. MS (m/z): 261.1 (MH⁺).

### 2-(3-Formyl-5-methoxy-2-methyl-indol-1-yl)-N,N-dimethyl-acetamide

Reaction conditions: room temperature for 18 hours. Purified by silica gel column chromatography (eluent: gradient from CHCl₃ to CHCl₃/MeOH 95:5). Yield: 82%. MS (m/z): 275.1 (MH⁺).

### 1-Dimethylcarbamoylmethyl-3-formyl-5-methoxy-indole-2-carboxylic acid dimethylamide

Reaction conditions: MW heating (250 W, 20 minutes, 80° C.). Purified by silica gel column chromatography (eluent: gradient from CHCl₃/MeOH 10:0 to 9:1). Yield: 59%. MS (m/z): 332.4 (MH⁺).

### 2-(2-Cyclopropyl-3-formyl-5-methoxy-indole-1-yl)-N,N-dimethyl-acetamide

Reaction conditions: 60° C. for 48 hours. Purified by silica gel column chromatography (eluent: gradient from petroleum ether/EtOAc 1:1 to EtOAc). Yield: 24%. MS (m/z): 301.2 (MH⁺).

### 2-(2-Trifluoromethyl-3-formyl-5-methoxy-indole-1-yl-N,N-dimethyl-acetamide

Reaction conditions: 60° C. for 48 hours. Purified by silica gel column chromatography (eluent: gradient from petroleum ether/EtOAc 5:5 to 0:10). Yield: 58%. MS (m/z): 329.3 (MH⁺).

### 2-[3-Formyl-5-methoxy-2-(1-methyl-1H-pyrazol-4-yl)-indol-1-yl]-N,N-dimethyl-acetamide

Reaction conditions: room temperature for 24 hours. Purified by silica gel column chromatography (eluent: CHCl₃). Yield: 60%. MS (m/z): 341.1 (MH⁺).

### General procedure for the alkylation with 1-bromo-2-chloroethane

NaH (60% dispersion in mineral oil, 1.2 g, 29.2 mmol, 2 eq.) was added to a solution of the selected 5-methoxy-indole-3-carbaldehyde 1× (14.6 mmol, 1 eq.) in DMF (250 mL), cooled to 0° C. The resulting suspension was stirred for 15 minutes, and then 1-bromo-2-chloro-ethane (6.1 mL, 73 mmol, 5 eq.) was added. The ice was removed and the mixture was stirred under the condition indicated below. The reaction was quenched with the addition of water and extracted with EtOAc. The organic layer was washed with water and brine, dried on Na₂SO₄, and evaporated to give a crude mixture that was purified by silica gel column chromatography. According to this procedure, the following compounds were obtained.

### 1-(2-Chloro-ethyl)-5-methoxy-indole-3-carbaldehyde

Reaction conditions: room temperature for 12 hours. Purified by silica gel column chromatography (eluent: CHCl₃). Yield*: 56%. MS (m/z): 238.3 (MH⁺).

### 1-(2-Chloro-ethyl)-5-methoxy-2-methyl-indole-3-carbaldehyde

Reaction conditions: 90° C. for 4 days, fresh 1-bromo-2-chloro-ethane (2.5 eq.) added every 12 hours. Purified by silica gel column chromatography (eluent: gradient from hexane:EtOAc 7:3 to hexane/EtOAc 1:1). Yield*: 61%. MS (m/z): 252.2 (MH⁺).

### 1-(2-Chloro-ethyl)-3-formyl-5-methoxy-indole-2-carboxylic acid dimethyl amide

Reaction conditions: room temperature for 48 hours. Purified by silica gel column chromatography (eluent: MeOH/CHCl₃ 0.75:99.25). Yield*: 60%. MS (m/z): 309.1 (MH⁺).

### 1-(2-Chloro-ethyl)-2-cyclopropyl-5-methoxy-indole-3-carbaldehyde

Reaction conditions: 90° C. for 4 days, fresh 1-bromo-2-chloro-ethane (2.5 eq.) added every 12 hours. Purified by silica gel column chromatography (eluent: methylene chloride/MeOH 98:2). Yield*: 13%. MS (m/z): 278.2 (MH⁺).

### 1-(2-Chloro-ethyl)-5-methoxy-2-(morpholine-4-carbonyl)-indole-3-carbaldehyde

Reaction conditions: room temperature for 12 hours. Purified by silica gel column chromatography (eluent: MeOH/CHCl₃ 1:99). Yield*: 70%. MS (m/z): 351.2 (MH⁺).

### 1-(2-Chloro-ethyl)-5-methoxy-2-(pyrrolidine-4-carbonyl)-indole-3-carbaldehyde

Reaction conditions: room temperature for 12 hours. Purified by silica gel column chromatography (eluent: MeOH/CHCl₃ 1:99). Yield*: 70%. MS (m/z): 335.2 (MH⁺). *Yields were calculated assuming the product as only chloro derivative (the bromo derivative is usually <30%).

### General Procedure for the Nucleophilic Displacement

To a solution of the selected 1-(2-chloro-ethyl)-5-methoxy-indole-3-carbaldehyde 3× (8.6 mmol, 1 eq.) in acetonitrile (180 mL), K₂CO₃ (5.94 g, 43.0 mmol, 5 eq.), KI (3.57 g, 21.5 mmol, 2.5 eq.) and the nucleophile (34.4 mmol, 4 eq.) were added. The resulting suspension was stirred at 90° C. for 48 hours, then water and EtOAc were added. The layers were separated and the aqueous layer was extracted with EtOAc. Combination of the organic layers, followed by drying on Na₂SO₄ and evaporation, afforded the crude product. According to this procedure, the following compounds were obtained.

### 5-Methoxy-1-[2-(4-methyl-piperazin-1-yl)-ethyl]-indole-3-carbaldehyde

Nucleophile: N-methyl-piperazine. Purified by silica gel column chromatography (eluent: CHCl₃/MeOH 98:2). Yield: 51%. MS (m/z): 302.4 (MH⁺).

### 1-[2-(3-Hydroxy-pyrrolidin-1-yl)-ethyl]-5-methoxy-indole-3-carbaldehyde

Nucleophile: pyrrolidin-3-ol. Purified by silica gel column chromatography (eluent: CHCl₃/MeOH 95:5). Yield: 66%. MS (m/z): 289.2 (MH⁺).

### 1-[2-(4-Hydroxy-piperidin-1-yl)-ethyl]-5-methoxy-indole-3-carbaldehyde

Nucleophile: piperidin-4-ol. Purified by silica gel column chromatography (eluent: CHCl₃/MeOH 95:5). Yield: 55%. MS (m/z): 303.4 (MH⁺).

### 5-Methoxy-2-methyl-1-[2-(4-methyl-piperazin-1-yl)-ethyl]-indole-3-carbaldehyde

Nucleophile: N-methyl-piperazine. Purified by silica gel column chromatography (eluent: CH₂Cl₂/MeOH 98:2+0.5% NH₃ aqueous.). Yield: 40%. MS (m/z): 316.2 (MH⁺).

### 1-[2-(4-Hydroxy-piperidin-1-yl)-ethyl]-5-methoxy-2-methyl-indole-3-carbaldehyde

Nucleophile: piperidin-4-ol. Purified by silica gel column chromatography (eluent: gradient from CHCl₃ to CHCl₃/MeOH 95:5). Yield: 47%. MS (m/z): 317.2 (MH⁺).

### 5-Methoxy-2-methyl-1-(2-pyrrolidin-1-yl-ethyl)-indole-3-carbaldehyde

Nucleophile: pyrrolidine. Purified by silica gel column chromatography (eluent: CHCl₃/MeOH 98:2). Yield: 35%. MS (m/z): 287.1 (MH⁺).

### 5-Methoxy-2-methyl-1-(2-piperidin-1-yl-ethyl)-indole-3-carbaldehyde

Nucleophile: piperidine. Purified by silica gel column chromatography (eluent: CHCl₃/MeOH 98:2). Yield: 50%. MS (m/z): 301.1 (MH⁺).

### 3-Formyl-5-methoxy-1-[2-(4-methyl-piperazin-1-yl)-ethyl]-indole-2-carboxylic acid dimethylamide

Nucleophile: N-methyl-piperazine. Purified by silica gel column chromatography (eluent: CHCl₃/MeOH 95:5). Yield: 62%. MS (m/z): 373.2 (MH⁺).

### 3-Formyl-1-[2-(3-hydroxy-pyrrolidin-1-yl)-ethyl]-5-methoxy-indole-2-carboxylic acid dimethylamide

Nucleophile: pyrrolidin-3-ol. Purified by silica gel column chromatography (eluent: CHCl₃/MeOH 95:5). Yield: 86%. MS (m/z): 360.1 (MH⁺).

### 3-Formyl-1-[2-(4-hydroxy-piperidin-1-yl)-ethyl]-5-methoxy-indole-2-carboxylic acid dimethylamide

Nucleophile: piperidin-4-ol. Purified by silica gel column chromatography (eluent: CHCl₃/MeOH 95:5). Yield: 69%. MS (m/z): 374.2 (MH⁺).

### 2-Cyclopropyl-5-methoxy-1-(2-(4-methylpiperazin-1-yl)ethyl)-indole-3-carbaldehyde

Nucleophile: N-methyl-piperazine. Purified by silica gel column chromatography (eluent: methylene chloride/MeOH 9:1). Yield: 28%. MS (m/z): 342.5 (MH⁺).

### 5-Methoxy-1-[2-(4-methyl-piperazin-1-yl)-ethyl]-2-(morpholine-4-carbonyl)-indole-3-carbaldehyde

Nucleophile: N-methyl piperazine. Purified by silica gel column chromatography (eluent: CHCl₃/MeOH 94:6). Yield: 45%. MS (m/z): 415.3 (MH⁺).

### 5-Methoxy-1-[2-(4-methyl-piperazin-1-yl)-ethyl]-2-(pyrrolidine-4-carbonyl)-indole-3-carbaldehyde

Nucleophile: N-methyl piperazine. Purified by silica gel column chromatography (eluent: CHCl₃/MeOH 94:6). Yield: 67%. MS (m/z): 399.4 (MH⁺).

### General procedure for the alkylation with 2-(2-bromo-ethoxy)-tetrahydro-pyran

NaH (1.76 g of 60% dispersion in mineral oil, 44 mmol, 2 eq.) was pre-washed with hexane and suspended in dry DMF (4 mL) under nitrogen. The suspension was cooled with an ice bath and a solution of the selected 5-methoxy-indole-3-carbaldehyde 1× (22 mmol, 1 eq.) in dry DMF (8 mL) was added by drops over 15 minutes. The cooling bath was removed and the mixture was stirred for 30 minutes. The reaction mixture was cooled again and a solution of 2-(2-bromo-ethoxy)-tetrahydro-pyran (6.0 g, 28.6 mmol, 1.3 eq.) in dry DMF (8 mL) was added by drops over 10 minutes. The reaction mixture was stirred according to the conditions indicated below. Then, the solvent was evaporated and the residue was partitioned between EtOAc and water. The combined organic layers were washed with water and brine and dried on Na₂SO₄. Evaporation of the solvent afforded a crude mixture that was purified by silica gel column chromatography. According to this procedure, the following compounds were obtained.

### 5-Methoxy-2-methyl-1-[2-(tetrahydro-pyran-2-yloxy)-ethyl]-indole-3-carbaldehyde

Reaction conditions: room temperature for 18 hours. Purified by silica gel column chromatography (eluent: gradient from CHCl₃ to CHCl₃/MeOH 95:5). Yield: 39%. MS (m/z): 318.2 (MH⁺).

### 2-Cyclopropyl-5-methoxy-1-[2-(tetrahydro-pyran-2-yloxy)-ethyl]-indole-3-carbaldehyde

Reaction conditions: 60° C. for 48 hours. The crude product was used without further purification. Yield: 76%. MS (m/z): 344.1 (MH⁺).

### 2-(Trifluoromethyl)-5-methoxy-1-(2-(tetrahydro-2H-pyran-2-loxy)ethyl)-indole-3-carbaldehyde

Reaction conditions: 60° C. for 48 hours. The crude product was used without further purification. MS (m/z): 372.2 (MH⁺).

### 5-Methoxy-2-(1-methyl-1H-pyrazol-4-yl)-1-[2-(tetrahydro-pyran-2-yloxy)-ethyl]-indole-3-carbaldehyde

Reaction conditions: room temperature for 2 days. Purified by silica gel column chromatography (eluent: CHCl₃/MeOH 98:2). Yield: 49%. MS (m/z): 384.2 (MH⁺).

### 2-(3,5-Dimethyl-isoxazol-4-yl)-5-methoxy-1-[2-(tetrahydro-pyran-2-yloxy)-ethyl]-indole-3-carbaldehyde

Reaction conditions: room temperature for 2 days. Purified by silica gel column chromatography (eluent: CHCl₃/MeOH 98:2). Yield: 51%. MS (m/z): 399.2 (MH⁺).

### 5-Methoxy-2-pyrimidin-5-yl-1-[2-(tetrahydro-pyran-2-yloxy)-ethyl]-indole-3-carbaldehyde

Reaction conditions: room temperature for 18 hours. The crude product was directly used for the following reaction. Yield: 87%. MS (m/z): 382.3 (MH⁺).

### 2-Cyclopentyl-5-methoxy-1-[2-(tetrahydro-pyran-2-yloxy)-ethyl]-indole-3-carbaldehyde

Reaction conditions: 60° C. for 48 hours. The crude product was used without further purification. Yield: 76%. MS (m/z): 372.4 (MH⁺).

### 2-Cyclohexyl-5-methoxy-1-[2-(tetrahydro-pyran-2-yloxy)-ethyl]-indole-3-carbaldehyde

Reaction conditions: room temperature for 18 hours. The crude product was directly used for the following reaction. Yield: 87%. MS (m/z): 386.5 (MH⁺).

### 2-Cyclobutyl-5-methoxy-1-[2-(tetrahydro-pyran-2-yloxy)-ethyl]-indole-3-carbaldehyde

Reaction conditions: room temperature for 18 hours. The crude product was directly used for the following reaction. Yield: 87%. MS (m/z): 358.0 (MH⁺).

### General Procedure for the Cleavage of the THP Group

To a solution of the selected 4× (1.5 mmol) in EtOH (10 mL), conc. HCl (0.5 mL) was added. The resulting suspension was stirred for 2 hours, and then water and EtOAc were added. The layers were separated and the aqueous layer was extracted with EtOAc. Combination of the organic layers, followed by drying on Na₂SO₄ and evaporation, afforded the crude product that was further purified as described below. According to this procedure, the following compounds were obtained.

### 1-(2-Hydroxy-ethyl)-5-methoxy-2-methyl-indole-3-carbaldehyde

Purified by trituration with Et₂O. Yield: 85%. MS (m/z): 234.2 (MH⁺).

### 2-Cyclopropyl-1-(2-hydroxy-ethyl)-5-methoxy-indole-3-carbaldehyde

Purified by triturated with Et₂O and silica gel column chromatography (eluent: hexane/EtOAc 1:1). Yield: 45%. MS (m/z): 260.1 (MH⁺).

### 2-(Trifluoromethyl)-1-(2-hydroxyethyl)-5-methoxy-indole-3-carbaldehyde

Purified by silica gel column chromatography (eluent: petroleum ether/EtOAc 8:2). Yield: 37%. MS (m/z): 288.1 (MH⁺).

### 1-(2-Hydroxy-ethyl)-5-methoxy-2-(1-methyl-1H-pyrazol-4-yl)-indole-3-carbaldehyde

Purified by trituration with Et₂O. Yield: 75%. MS (m/z): 300.2 (MH⁺).

### 2-(3,5-Dimethyl-isoxazol-4-yl)-1-(2-hydroxy-ethyl)-5-methoxy-indole-3-carbaldehyde

Purified by trituration with Et₂O. Yield: 85%. MS (m/z): 315.3 (MH⁺).

### 1-(2-Hydroxy-ethyl)-5-methoxy-2-pyrimidin-5-yl-indole-3-carbaldehyde

The crude product was used without further purification. Yield: 89%. MS (m/z): 298.2 (MH⁺).

### 2-Cyclopentyl-1-(2-hydroxy-ethyl)-5-methoxy-indole-3-carbaldehyde

Purified by silica gel column chromatography (eluent: petroleum ether/EtOAc 7:3). Yield (two steps from 1p): 48%. MS (m/z): 288.3 (MH⁺).

### 2-Cyclohexyl-1-(2-hydroxy-ethyl)-5-methoxy-indole-3-carbaldehyde

Purified by silica gel column chromatography (eluent: petroleum ether/EtOAc 7:3). Yield (two steps from 1q): 54%. MS (m/z): 302.4 (MH⁺).

### 2-Cyclobutyl-1-(2-hydroxy-ethyl)-5-methoxy-indole-3-carbaldehyde

Purified by silica gel column chromatography (eluent: petroleum ether/EtOAc 7:3). Yield (two steps from 1r): 42%. MS (m/z): 274.3 (MH⁺).

### General Procedure for the Preparation of the Intermediate Tosyl Esters

To a solution of the selected ester (1.12 mmol, 1 eq.) in dry methylene chloride (10 mL), Et₃N (0.24 mL, 1.7 mmol, 1.5 eq.) and DMAP (catalytic amount) were added at 0° C. After 10 minutes, TsCl (229 mg, 1.2 mmol, 1.07 eq.) was slowly added. The solution was stirred at room temperature overnight, and then the reaction mixture was diluted with methylene chloride and washed with water. The organic layer was dried on Na₂SO₄ and evaporated to give the crude product that was purified as indicated below. According to this procedure, the following compounds were obtained.

### Toluene-4-sulfonic acid 2-(3-formyl-5-methoxy-2-methyl-indol-1-yl)-ethyl ester

Purified by trituration with Et₂O. Yield: 85%. MS (m/z): 388.2 (MH⁺).

### Toluene-4-sulfonic acid 2-(2-cyclopropyl-3-formyl-5-methoxy-indol-1-yl)-ethyl ester

Purified by trituration with Et₂O. Yield: 66%. MS (m/z): 414.3 (MH⁺).

### Toluene-4-sulfonic acid 2-(3-formyl-5-methoxy-2-trifluoromethyl-indol-1-yl)-ethyl ester

The crude product was used without further purification. Yield: 92%. MS (m/z): 442.5 (MH⁺).

Toluene-4-sulfonic acid 2-[3-formyl-5-methoxy-2-(1-methyl-1H-pyrazol-4-yl)-indol-1-yl]-ethyl ester

Purified by silica gel column chromatography (eluent: CHCl₃/CH₃OH 98:2). Yield: 57%. MS (m/z): 454.2 (MH⁺).

### Toluene-4-sulfonic acid 2-[2-(3,5-dimethyl-isoxazol-4-yl)-3-formyl-5-methoxy-indol-1-yl]-ethyl ester

Purified by silica gel column chromatography (eluent: EtOAc/hexane 1:4). Yield: 53%. MS (m/z): 469.3 (MH⁺).

### Toluene-4-sulfonic acid 2-(3-formyl-5-methoxy-2-pyrimidin-5-yl-indol-1-yl)-ethyl ester

Purified by silica gel column chromatography (eluent: MeOH/CHCl₃ 0.5:99.5). Yield: 74%. MS (m/z): 452.2 (MH⁺).

### Toluene-4-sulfonic acid 2-(2-cyclopentyl-3-formyl-5-methoxy-indol-1-yl)-ethyl ester

The crude product was used without further purification. MS (m/z): 442.5 (MH⁺).

### Toluene-4-sulfonic acid 2-(2-cyclohexyl-3-formyl-5-methoxy-indol-1-yl)-ethyl ester

The crude product was used without further purification. MS (m/z): 456.1 (MH⁺).

### Toluene-4-sulfonic acid 2-(2-cyclobutyl-3-formyl-5-methoxy-indol-1-yl)-ethyl ester

The crude product was used without further purification. MS (m/z): 428.4 (MH⁺).

### General Procedures (A-D) for the Nucleophilic Displacement of the Tosylate Compounds

Procedure A

To a solution of the tosylate (0.74 mmol, 1 eq.) in acetonitrile (15 mL), K₂CO₃ (510 mg, 3.7 mmol, 5 eq.), KI (307 mg, 1.85 mmol, 2.5 eq.) and the selected nucleophile (2.96 mmol, 4 eq.) were added. The resulting suspension was stirred at 90° C. for 48 hours, and then water and EtOAc were added. The layers were separated and the aqueous layer was extracted with EtOAc. Combination of the organic layers, followed by drying on Na₂SO₄ and evaporation, afforded the crude product that was purified as described below. According to this procedure, the following compounds were obtained.

### 2-Cyclopropyl-1-(2-(3-hydroxypyrrolidin-1-yl)ethyl)-5-methoxy-indole-3-carbaldehyde

Nucleophile: pyrrolidin-3-ol. Purified by silica gel column chromatography (eluent: methylene chloride/MeOH 9:1). Yield: 54%. MS (m/z): 329.1 (MH⁺).

### 2-cyclopropyl-1-[2-(4-hydroxy-piperidin-1-yl)-ethyl]-5-methoxy-indole-3-carbaldehyde

Nucleophile: piperidin-4-ol. Purified by silica gel column chromatography (eluent: methylene chloride/MeOH 9:1). Yield: 46%. MS (m/z): 343.5 (MH⁺).

### 2-Trifluoromethyl-5-methoxy-1-(2-(4-methylpiperazin-1-yl)ethyl)-indole-3-carbaldehyde

Nucleophile: N-methyl-piperazine. Purified by silica gel column chromatography (eluent: petroleum ether/EtOAc 2:8, then methylene chloride/MeOH 9:1). Yield: 32%. MS (m/z): 370.2 (MH⁺).

Procedure B

Tosylate (2.06 mmol, 1 eq.) was dissolved in DMF (8 mL) and the selected nucleophile (8.26 mmol, 4 eq.) was added. The resulting solution was heated at 100° C. by microwave irradiation for 20 minutes. DMF was evaporated and the residue was purified as described below. According to this procedure, the following compounds were obtained.

### 1-(2-Imidazol-1-yl-ethyl)-5-methoxy-2-methyl-indole-3-carbaldehyde

Nucleophile: imidazole. Purified by silica gel column chromatography (eluent: CHCl₃/MeOH 98:2). Yield: 70%. MS (m/z): 284.1 (MH⁺).

### 1-[2-(3-Hydroxyl-pyrrolidin-1-yl)-ethyl]-5-methoxy-2-methyl-indole-3-carbaldehyde

Nucleophile: pyrrolidin-3-ol. Purified by silica gel column chromatography (eluent: CHCl₃/MeOH 98:2). Yield: 62%. MS (m/z): 303.2 (MH⁺).

### 5-Methoxy-2-methyl-1-[2-(2-methyl-pyrrolidin-1-yl)-ethyl]-indole-3-carbaldehyde

Nucleophile: 2-methylpyrrolidine. Purified by silica gel column chromatography (eluent: CHCl₃/MeOH 98:2). Yield: 52%. MS (m/z): 301.3 (MH⁺).

### 5-Methoxy-2-methyl-1-[2-(4-methyl-piperidin-1-yl)-ethyl]-indole-3-carbaldehyde

Nucleophile: 4-methyl piperidine. Purified by silica gel column chromatography (eluent: CHCl₃/MeOH 98:2). Yield: 52%. MS (m/z): 315.2 (MH⁺).

### 1-(2-Azepan-1-yl-ethyl)-5-methoxy-2-methyl-indole-3-carbaldehyde

Nucleophile: azepane. Purified by silica gel column chromatography (eluent: CHCl₃/MeOH 98:2). Yield: 58%. MS (m/z): 315.2 (MH⁺).

### 5-Methoxy-1-[2-(4-methyl-piperazin-1-yl)-ethyl]-2-(1-methyl-1H-pyrazol-4-yl)-indole-3-carbaldehyde

Nucleophile: N-methyl-piperazine. Purified by silica gel column chromatography (eluent: CHCl₃/MeOH 99:1 to 97:3). Yield: 40%. MS (m/z): 382.4 (MH⁺).

### 2-(3,5-Dimethyl-isoxazol-4-yl)-5-methoxy-1-[2-(4-methyl-piperazin-1-yl)-ethyl]-indole-3-carbaldehyde

Nucleophile: N-methyl-piperazine. Purified by silica gel column chromatography (eluent: CHCl₃/MeOH 98:2). Yield: 49%. MS (m/z): 397.2 (MH⁺).

### 5-Methoxy-1-[2-(4-methyl-piperazin-1-yl)-ethyl]-2-pyrimidin-5-yl-indole-3-carbaldehyde

Nucleophile: N-methyl-piperazine. Purified by silica gel column chromatography (eluent: CHCl₃/MeOH 98:2). Yield: 63%. MS (m/z): 380.3 (MH⁺).

Procedure C

NaH (60% dispersion in mineral oil, 1.2 g, 0.56 mmol, 1.1 eq.) was added to a solution of the selected nucleophile (0.51 mmol, 1 eq.) in DMF (10 mL) cooled to 0° C. The resulting suspension was stirred for 45 minutes, and then tosylate 6× (0.87 mmol, 1.7 eq.) was added. The ice bath was removed and the mixture was heated at 50° C. overnight. After cooling to room temperature, the reaction was partitioned between water and EtOAc. The organic layer was washed with water and brine, dried on Na₂SO₄ and evaporated under reduced pressure. The crude mixture was purified as described below. According to this procedure, the following compounds were obtained.

### 2-Cyclopropyl-1-(2-imidazol-1-yl-ethyl)-5-methoxy-indole-3-carbaldehyde

Nucleophile: imidazole. Purified by silica gel column chromatography (eluent: petroleum ether/EtOAc 4:6, then methylene chloride/MeOH 95:5). Yield: 34%. MS (m/z): 310.4 (MH⁺). ¹H NMR (300 MHz, CDCl₃): 10.38 (s, 1H); 7.90 (bs, 1H); 7.22-7.13 (m, 2H); 7.03-6.92 (m, 2H); 6.46 (s, 1H); 4.64 (t, 2H); 4.39 (t, 2H); 3.91 (s, 3H); 1.89-1.53 (bs, 1H); 1.11-1.03 (m, 2H); 0.77-0.70 (m, 2H).

### 2-Cyclopropyl-5-methoxy-1-(2-pyrazol-1-yl-ethyl)-indole-3-carbaldehyde

Nucleophile: pyrazole. Purified by silica gel column chromatography (eluent: petroleum ether/EtOAc 3:7). Yield: 74%. MS (m/z): 310.3 (MH⁺).

### 5-Methoxy-1-(2-pyrazol-1-yl-ethyl)-2-trifluoromethyl-indole-3-carbaldehyde

Nucleophile: pyrazole. Purified by silica gel column chromatography (eluent: petroleum ether/EtOAc 3:7). Yield: 19%. MS (m/z): 338.3 (MH⁺).

### 2-Cyclopentyl-5-1-[2-(4-methylpiperazin-1-yl)ethyl]-indole-3-carbaldehyde

Nucleophile: N-methyl piperazine. Purified by silica gel column chromatography (eluent: petroleum ether/EtOAc 2:8). Yield: 38%. MS (m/z): 370.3 (MH⁺).

### 2-Cyclohexyl-5-1-[2-(4-methylpiperazin-1-yl)ethyl]-indole-3-carbaldehyde

Nucleophile: N-methyl piperazine. Purified by silica gel column chromatography (eluent: dichloromethane/MeOH 20:1). Yield: 86%. MS (m/z): 384.3 (MH⁺).

### 2-Cyclobutyl-5-1-[2-(4-methylpiperazin-1-yl)ethyl]-indole-3-carbaldehyde

Nucleophile: N-methyl piperazine. Purified by silica gel column chromatography (eluent: dichloromethane/MeOH 95:5). Yield: 78%. MS (m/z): 356.3 (MH⁺).

### General procedure for the alkylation with 1-bromo-3-chloro-propane

To a solution of the selected 5-methoxy-indole-3-carbaldehyde 1× (24.6 mmol) in DMF (90 mL), cooled to 0° C., NaH (60% dispersion in mineral oil, 1.97 g, 49.3 mmol, 2 eq.) was added. The resulting suspension was stirred for 15 minutes, and then 1-bromo-3-chloro-propane (12.2 mL, 123.1 mmol, 5 eq.) was added. The ice was removed and the reaction mixture was allowed to stir overnight at room temperature. The reaction was quenched with the addition of water and extracted with EtOAc. The organic layer was washed with brine, dried on Na₂SO₄ and evaporated to give a crude mixture that was further purified as described below. According to this procedure, the following compounds were obtained.

### 1-(3-Chloro-propyl)-5-methoxy-indole-3-carbaldehyde

Purified by silica gel column chromatography (eluent: gradient from hexane/EtOAc 7:3 to hexane/EtOAc 1:1). Yield 86%. MS (m/z): 252.1 (MH⁺).

### 1-(3-Chloro-propyl)-5-methoxy-2-methyl-indole-3-carbaldehyde

Purified by silica gel column chromatography (eluent: CHCl₃/MeOH 99.8:0.2). Yield*: 78%. MS (m/z): 266.1 (MH⁺).

### 1-(3-Chloro-propyl)-3-formyl-5-methoxy-indole-2-carboxylic acid dimethyl amide

Purified by silica gel column chromatography (eluent: CHCl₃/MeOH 99:1). Yield*: 53%. MS (m/z): 323.2 (MH⁺).

### 1-(3-Chloro-propyl)-2-cyclopropyl-5-methoxy-indole-3-carbaldehyde

Purified by silica gel column chromatography (eluent: petroleum ether/EtOAc 7:3). Yield*: 57%. MS (m/z): 292.3 (MH⁺). *Yields were calculated assuming the product as only chloro derivative.

### General Procedures (A, B) for the Nucleophilic Displacement (Preparation of Carbaldehyde Compounds

Procedure A

To a solution of 7× (21.24 mmol, 1 eq.) in acetonitrile (350 mL), K₂CO₃ (14.66 g, 106.2 mmol, 5 eq.), KI (8.82 g, 53.1 mmol, 2.5 eq.) and dimethylamine (2M in THF, 42.5 mL, 84.96 mmol, 4 eq.) were added. The resulting suspension was heated to 90° C. for 24 hours. The reaction mixture was allowed to cool to room temperature and filtered. The recovered solid was washed with EtOAc. To the filtrate water was added, the layers were separated and the aqueous layer was extracted with EtOAc. Combination of the organic layers, followed by drying on Na₂SO₄ and evaporation, afforded a crude mixture that was further purified as described below. According to this procedure, the following compounds were obtained.

### 1-(3-Dimethylamino-propyl)-5-methoxy-indole-3-carbaldehyde

Purified by silica gel column chromatography (eluent: CH₂Cl₂/MeOH 98:2+0.5% NH₃ aqueous.). Yield: 71%. MS (m/z): 261.1 (MH⁺).

### 1-(3-Dimethylamino-propyl)-5-methoxy-2-methyl-indole-3-carbaldehyde

Purified by silica gel column chromatography (eluent: CHCl₃/MeOH 95:5). Yield: 83%. MS (m/z): 275.4 (MH⁺).

### 1-(3-Dimethylamino-propyl)-3-formyl-5-methoxy-indole-2-carboxylic acid dimethylamide

Purified by silica gel column chromatography (eluent: CHCl₃/MeOH 96:4). Yield: 73%. MS (m/z): 332.2 (MH⁺).

### 2-Cyclopropyl-1-(3-dimethylamino-propyl)-5-methoxy-indole-3-carbaldehyde

Purified by silica gel column chromatography (eluent: CH₂Cl₂/MeOH 99:1+0.5% NH₃ aqueous). Yield: 80%. MS (m/z): 301.1 (MH⁺).

Procedure B

1-(3-Chloro-propyl)-5-methoxy-2-methyl-indole-3-carbaldehyde (7b, 0.50 g, 1.879 mmol, 1 eq.) and the selected nucleophile (16.91 mmol, 9 eq.) were heated at 80° C. by microwave irradiation for 15 minutes. Excess nucleophile was evaporated and the crude mixture was further purified as indicated below. According to this procedure, the following compounds were obtained.

### 5-Methoxy-2-methyl-1-(3-pyrrolidin-1-yl-propyl)-indole-3-carbaldehyde

Nucleophile: pyrrolidine. Purified by silica gel column chromatography (eluent: CHCl₃/MeOH 97:3). Yield: 33%. MS (m/z): 301.3 (MH⁺).

### 5-Methoxy-2-methyl-1-[3-(2-methyl-pyrrolidin-1-yl)-propyl]-indole-3-carbaldehyde

Nucleophile: 2-methylpyrrolidine. Purified by silica gel column chromatography (eluent: CHCl₃/MeOH 98:2). Yield: 71%. MS (m/z): 315.3 (MH⁺).

### 5-Methoxy-2-methyl-1-(3-piperidin-1-yl-propyl)-indole-3-carbaldehyde

Nucleophile: piperidine. Purified by silica gel column chromatography (eluent: CHCl₃/MeOH 96:4). Yield: 70%. MS (m/z): 315.2 (MH⁺).

### 5-Methoxy-2-methyl-1-[3-(4-methyl-piperidin-1-yl)-propyl]-indole-3-carbaldehyde

Nucleophile: 4-methyl piperidine. Purified by silica gel column chromatography (eluent: CHCl₃/MeOH 96:4). Yield: 89%. MS (m/z): 329.1 (MH⁺).

### 1-(3-Azepan-1-yl-propyl)-5-methoxy-2-methyl-indole-3-carbaldehyde

Nucleophile: azepane. Purified by silica gel column chromatography (eluent: CHCl₃/MeOH 96:4). Yield: 64%. MS (m/z): 329.1 (MH⁺).

### Synthesis of other indole-3-carbaldehydes

### Preparation of 1-methyl-2-phenyl-1H-indole-3-carbaldehyde

To a solution of 2-phenyl-1H-indole-3-carbaldehyde (7.41 g, 33.5 mmol) in DMF (50 ml) cooled to 0° C. was added in portions, sodium hydride (2.68 g, 67.0 mmol). After stirring for 30 minutes, iodomethane (4.18 ml, 67.0 mmol) was added and the reaction stirred for 30 minutes, then allowed to warm to room temperature and stirred overnight. Water (150 mL) was added and the resulting solid was filtered, washed well with water and air dried to give a light green solid 1-methyl-2-phenyl-1H-indole-3-carbaldehyde (5.30 g, 22.53 mmol, 67.3% yield), MS (m/z) 236.3 (MH⁺).

### Preparation of 4-(4-methoxyphenyl)-1H-indole-3-carbaldehyde

To a mixture of 4-bromo-1H-indole-3-carbaldehyde (112 mg, 0.5 mmol), Tetrakis(triphenylphosphine)palladium(0) (57.8 mg, 0.050 mmol), 2-(4-methoxyphenyl)-4,4,5,5-tetramethyl-1,3,2-dioxaborolane (129 mg, 0.550 mmol) and dimethoxyethane (3.0 mL) in a 2-5 mL microwave tube was added 0.75 mL of 2M sodium carbonate (1.5 mmol). This was capped and heated in the microwave for 1 hour at 110° C. Work-up by quenching into 20 mL water, mixture extracted with ethyl acetate (2×10 mL) the ethyl acetate layer evaporated to give a gum which was dissolved dichloromethane passed through a short pad of silica-gel, the product was removed from the silica gel eluting with 1:1 hexane/ethyl acetate then evaporated to give 4-(4-methoxyphenyl)-1H-indole-3-carbaldehyde (130 mg, 0.517 mmol, 103% yield). Used as is for the next step.

### Preparation of 4-(4-methoxyphenyl)-1-methyl-1H-indole-3-carbaldehyde

To a mixture of 4-bromo-1-methyl-1H-indole-3-carbaldehyde (238 mg, 1.0 mmol), Tetrakis(triphenylphosphine)palladium(0) (116 mg, 0.100 mmol), 2-(4-methoxyphenyl)-4,4,5,5-tetramethyl-1,3,2-dioxaborolane (258 mg, 1.100 mmol) and dimethoxyethane (3.0 mL) in a 2-5 mL microwave tube was added 1.125 mL of 2M sodium carbonate (1.5 mmol). This was capped and heated in the microwave for 1 hour at 120° C. Work-up by quenching into 20 mL water, mixture extracted with ethyl acetate (2×10 mL) the ethyl acetate layer evaporated to give a gum which was dissolved dichloromethane, loaded onto 2 grams of silica gel and purified by chromatography on the ISCO Companion™ using a hexane/ethyl acetate gradient on a 40 gram column, combined cuts containing product were then evaporated to give a gum. After standing overnight, the gum showed some crystals. This was treated with 6:1 hexanes/ethyl acetate, the off white solid was collected on a sintered glass funnel, washed with fresh solvent and air dried to give 4-(4-methoxyphenyl)-1-methyl-1H-indole-3-carbaldehyde (132 mg, 0.498 mmol, 49.8% yield), MS (m/z): 266.1 (MH⁺).

### III. Condensation of indole-3-carbaldehydes and benzofuranone compounds or benzothiophenone compounds

### 2-(1H-indol-3-ylmethylene)-1-benzofuran-3(2H)-one (Example 1)

To benzofuranone (15.6 mmol, 0.9 eq) and 3-indole aldehyde (17.3 mmol, 1 eq) in EtOH (2 mL) was added a catalytic amount of HCl (12 N). The resulting mixture was stirred for 120 minutes at 80° C. and allowed to cool to room temperature. The solution was concentrated in a Speed-Vac and the resulting residue purified via preparative HPLC conditions to afford the title compound. LCMS RT=2.40 MS=260.1.

### 4,6-dihydroxy-2-[(5-methoxy-2-phenyl-1H-indol-3-yl)methylene]-1-benzofuran-3(2H)-one (Example 70)

To the 4,6-dihydroxy-benzofuran-3-one (125 mgs, 0.75 mmol, 1 eq) and desired 5-methoxy-2-phenyl-1H-indole-3-carbaldehyde (188 mgs, 0.75 mmol, 1 eq) in EtOH (3 mL) was added a catalytic amount of HCl (12 N). The resulting mixture was stirred for 180 minutes at 80° C. and allowed to cool to room temperature. The suspension was filtered. The red solid was dried in a Speed-Vac and purified via preparative HPLC to afford the title compound. LCMS RT=2.19 MS=398.1.

### 4,6-dihydroxy-2-({5-methoxy-2-methyl-1-[2-(4-methylpiperazin-1-yl)ethyl]-1H-indol-3-yl}methylene)-1-benzofuran-3(2H)-one (Example 108)

To 1-(2-chloroethyl)-5-methoxy-2-methyl-1H-indole-3-carbaldehyde (crude product taken directly from previous reaction) in EtOH (3 mL) was added the desired 4,6-dihydroxy-benzofuran-3-one (70 mgs) and HCl (12N, 8 drops). The reaction mixture was heated to 90 C and stirred for 2.5 hrs—LCMS indicated no remaining benzofuranone and product formation. The reaction was allowed to cool. Concentration of the solution in a Speed-Vac and purification via preparative HPLC afforded the title compound. LCMS RT=1.89 MS=464.2.

Using the procedure of Example 1, 70, and 108 Examples 2-69, 71-107, 109-116, and 120-269 were also prepared. In some cases the reaction suspension was filtered and the solid recrystallized if necessary in EtOH. Otherwise the reaction was concentrated via Speed-Vac and purified via preparative HPLC to afford the desired compounds. Compound and analytical data are show in Table I below.

TABLE I
Compounds Prepared According the Procedure of Example 1.

| | | Time | | | |
|---|---|---|---|---|---|
| Example | Name | (min) | Mass | Ion | LCMS Conditions |
| 1 | 2-(1H-indol-3-ylmethylene)-1- | 2.45 | 260.1 | M − H | std method w/ |
| | benzofuran-3(2H)-one | | | | NH₄OAc |
| 2 | 2-[(2-phenyl-1H-indol-3- | 2.77 | 338.1 | M + H | std method w/ |
| | yl)methylene]-1-benzofuran-3(2H)- | | | | NH₄OAc |
| | one | | | | |
| 3 | 2-[(1-methyl-2-phenyl-1H-indol-3- | 2.95 | 352.1 | M + H | std method w/ |
| | yl)methylene]-1-benzofuran-3(2H)- | | | | NH₄OAc |
| | one | | | | |
| 4 | 2-[(1-methyl-1H-indol-3- | 2.66 | 276.1 | M + H | std method w/ |
| | yl)methylene]-1-benzofuran-3(2H)- | | | | NH₄OAc |
| | one | | | | |
| 5 | 6-hydroxy-2-(1H-indol-3- | 2.12 | 276.1 | M − H | std method w/ |
| | ylmethylene)-1-benzofuran-3(2H)- | | | | NH₄OAc |
| | one | | | | |
| 6 | 6-hydroxy-2-[(2-phenyl-1H-indol-3- | 2.41 | 352.1 | M − H | std method w/ |
| | yl)methylene]-1-benzofuran-3(2H)- | | | | NH₄OAc |
| | one | | | | |
| 7 | 6-hydroxy-2-[(1-methyl-2-phenyl- | 2.57 | 366.1 | M − H | std method w/ |
| | 1H-indol-3-yl)methylene]-1- | | | | NH₄OAc |
| | benzofuran-3(2H)-one | | | | |
| 8 | 6-hydroxy-2-[(1-methyl-1H-indol-3- | 2.48 | 290.1 | M − H | std method w/ |
| | yl)methylene]-1-benzofuran-3(2H)- | | | | NH₄OAc |
| | one | | | | |
| 9 | 2-(1H-indol-3-ylmethylene)-7- | 2.47 | 290.1 | M − H | std method w/ |
| | methoxy-1-benzofuran-3(2H)-one | | | | NH₄OAc |
| 10 | 7-methoxy-2-[(2-phenyl-1H-indol-3- | 2.77 | 368.1 | M + H | std method w/ |
| | yl)methylene]-1-benzofuran-3(2H)- | | | | NH₄OAc |
| | one | | | | |
| 11 | 7-methoxy-2-[(1-methyl-2-phenyl- | 2.95 | 382.1 | M + H | std method w/ |
| | 1H-indol-3-yl)methylene]-1- | | | | NH₄OAc |
| | benzofuran-3(2H)-one | | | | |
| 12 | 7-methoxy-2-[(1-methyl-1H-indol-3- | 2.66 | 306.1 | M + H | std method w/ |
| | yl)methylene]-1-benzofuran-3(2H)- | | | | formic |
| | one | | | | |
| 13 | 4,6-dihydroxy-2-(1H-indol-3- | 2.17 | 292.1 | M − H | std method w/ |
| | ylmethylene)-1-benzofuran-3(2H)- | | | | NH₄OAc |
| | one | | | | |
| 14 | 4,6-dihydroxy-2-[(2-phenyl-1H- | 2.28 | 368.1 | M − H | std method w/ |
| | indol-3-yl)methylene]-1- | | | | NH₄OAc |
| | benzofuran-3(2H)-one | | | | |
| 15 | 4,6-dihydroxy-2-[(1-methyl-2- | 2.45 | 384.1 | M + H | std method w/ |
| | phenyl-1H-indol-3-yl)methylene]-1- | | | | NH₄OAc |
| | benzofuran-3(2H)-one | | | | |
| 16 | (2Z)-4,6-dihydroxy-2-[(1-methyl- | 2.13 | 306.1 | M − H | std method w/ |
| | 1H-indol-3-yl)methylene]-1- | | | | NH₄OAc |
| | benzofuran-3(2H)-one | | | | |
| 17 | (2Z)-2-{[2-(4-chlorophenyl)-1H- | 2.81 | 370.1 | M − H | std method w/ |
| | indol-3-yl]methylene}-1- | | | | NH₄OAC |
| | benzofuran-3(2H)-one | | | | |
| 18 | (2Z)-2-{[2-(2-naphthyl)-1H-indol-3- | 2.88 | 388.1 | M + H | std method w/ |
| | yl]methylene}-1-benzofuran-3(2H)- | | | | NH₄OAC |
| | one | | | | |
| 19 | (2Z)-2-{[2-(4-fluorophenyl)-1H- | 2.7 | 354.1 | M − H | std method w/ |
| | indol-3-yl]methylene}-1- | | | | NH₄OAC |
| | benzofuran-3(2H)-one | | | | |
| 20 | (2Z)-2-[(2-methyl-5-nitro-1H-indol- | 2.6 | 319.1 | M − H | std method w/ |
| | 3-yl)methylene]-1-benzofuran- | | | | NH₄OAC |
| | 3(2H)-one | | | | |
| 21 | (2Z)-2-[(2-methyl-1H-indol-3- | 2.48 | 274.1 | M − H | std method w/ |
| | yl)methylene]-1-benzofuran-3(2H)- | | | | NH₄OAC |
| | one | | | | |
| 22 | (2Z)-2-[(6-methyl-1H-indol-3- | 2.5 | 274.1 | M − H | std method w/ |
| | yl)methylene]-1-benzofuran-3(2H)- | | | | NH₄OAC |
| | one | | | | |
| 23 | (2Z)-2-[(7-methyl-1H-indol-3- | 2.5 | 274.1 | M − H | std method w/ |
| | yl)methylene]-1-benzofuran-3(2H)- | | | | NH₄OAC |
| | one | | | | |
| 24 | (2Z)-2-[(5-bromo-1H-indol-3- | 2.62 | 338 | M − H | std method w/ |
| | yl)methylene]-1-benzofuran-3(2H)- | | | | NH₄OAC |
| | one | | | | |
| 25 | (2Z)-2-[(1-benzyl-1H-indol-3- | 2.81 | 352.1 | M + H | std method w/ |
| | yl)methylene]-1-benzofuran-3(2H)- | | | | NH₄OAC |
| | one | | | | |
| 26 | (2Z)-2-{[2-(4-chlorophenyl)-1H- | 2.46 | 386.1 | M − H | std method w/ |
| | indol-3-yl]methylene}-6-hydroxy-1- | | | | NH₄OAC |
| | benzofuran-3(2H)-one | | | | |
| 27 | (2Z)-6-hydroxy-2-{[2-(2-naphthyl)- | 2.53 | 402.1 | M − H | std method w/ |
| | 1H-indol-3-yl]methylene}-1- | | | | NH₄OAC |
| | benzofuran-3(2H)-one | | | | |
| 28 | (2Z)-2-{[2-(4-fluorophenyl)-1H- | 2.37 | 370.1 | M − H | std method w/ |
| | indol-3-yl]methylene}-6-hydroxy-1- | | | | NH₄OAC |
| | benzofuran-3(2H)-one | | | | |
| 29 | (2Z)-6-hydroxy-2-[(2-methyl-5-nitro- | 2.26 | 335.1 | M − H | std method w/ |
| | 1H-indol-3-yl)methylene]-1- | | | | NH₄OAC |
| | benzofuran-3(2H)-one | | | | |
| 30 | (2Z)-6-hydroxy-2-[(2-methyl-1H- | 2.11 | 290.1 | M − H | std method w/ |
| | indol-3-yl)methylene]-1- | | | | NH₄OAC |
| | benzofuran-3(2H)-one | | | | |
| 31 | (2Z)-6-hydroxy-2-[(6-methyl-1H- | 2.2 | 290.1 | M − H | std method w/ |
| | indol-3-yl)methylene]-1- | | | | NH₄OAC |
| | benzofuran-3(2H)-one | | | | |
| 32 | (2Z)-2-[(5-bromo-1H-indol-3- | 2.29 | 354 | M − H | std method w/ |
| | yl)methylene]-6-hydroxy-1- | | | | NH₄OAC |
| | benzofuran-3(2H)-one | | | | |
| 33 | (2Z)-2-[(1-benzyl-1H-indol-3- | 2.5 | 366.1 | M − H | std method w/ |
| | yl)methylene]-6-hydroxy-1- | | | | NH₄OAC |
| | benzofuran-3(2H)-one | | | | |
| 34 | (2Z)-2-{[5-(benzyloxy)-1H-indol-3- | 2.41 | 382.1 | M − H | std method w/ |
| | yl]methylene}-6-hydroxy-1- | | | | NH₄OAC |
| | benzofuran-3(2H)-one | | | | |
| 35 | (2Z)-2-{[2-(4-chlorophenyl)-1H- | 2.81 | 400.1 | M − H | std method w/ |
| | indol-3-yl]methylene}-7-methoxy-1- | | | | NH₄OAC |
| | benzofuran-3(2H)-one | | | | |
| 36 | (2Z)-7-methoxy-2-{[2-(2-naphthyl)- | 2.87 | 418.1 | M + H | std method w/ |
| | 1H-indol-3-yl]methylene}-1- | | | | NH₄OAC |
| | benzofuran-3(2H)-one | | | | |
| 37 | (2Z)-2-{[2-(4-fluorophenyl)-1H- | 2.7 | 384.1 | M − H | std method w/ |
| | indol-3-yl]methylene}-7-methoxy-1- | | | | NH₄OAC |
| | benzofuran-3(2H)-one | | | | |
| 38 | (2Z)-7-methoxy-2-[(2-methyl-5- | 2.57 | 349.1 | M − H | std method w/ |
| | nitro-1H-indol-3-yl)methylene]-1- | | | | NH₄OAC |
| | benzofuran-3(2H)-one | | | | |
| 39 | (2Z)-7-methoxy-2-[(2-methyl-1H- | 2.47 | 304.1 | M − H | std method w/ |
| | indol-3-yl)methylene]-1- | | | | NH₄OAC |
| | benzofuran-3(2H)-one | | | | |
| 40 | (2Z)-7-methoxy-2-[(6-methyl-1H- | 2.53 | 306.1 | M + H | std method w/ |
| | indol-3-yl)methylene]-1- | | | | NH₄OAC |
| | benzofuran-3(2H)-one | | | | |
| 41 | (2Z)-2-[(5-bromo-1H-indol-3- | 2.63 | 368 | M − H | std method w/ |
| | yl)methylene]-7-methoxy-1- | | | | NH₄OAC |
| | benzofuran-3(2H)-one | | | | |
| 42 | (2Z)-2-[(1-benzyl-1H-indol-3- | 2.85 | 382.6 | M + H | std method w/ |
| | yl)methylene]-7-methoxy-1- | | | | formic |
| | benzofuran-3(2H)-one | | | | |
| 43 | (2Z)-2-{[5-(benzyloxy)-1H-indol-3- | 2.7 | 398.1 | M + H | std method w/ |
| | yl]methylene}-7-methoxy-1- | | | | NH₄OAC |
| | benzofuran-3(2H)-one | | | | |
| 44 | (2Z)-2-{[2-(4-chlorophenyl)-1H- | 2.33 | 402.1 | M − H | std method w/ |
| | indol-3-yl]methylene}-4,6- | | | | NH₄OAC |
| | dihydroxy-1-benzofuran-3(2H)-one | | | | |
| 45 | (2Z)-4,6-dihydroxy-2-{[2-(2- | 2.43 | 418.1 | M − H | std method w/ |
| | naphthyl)-1H-indol-3-yl]methylene}- | | | | NH₄OAC |
| | 1-benzofuran-3(2H)-one | | | | |
| 46 | (2Z)-2-{[2-(4-fluorophenyl)-1H- | 2.25 | 386.1 | M − H | std method w/ |
| | indol-3-yl]methylene}-4,6- | | | | NH₄OAC |
| | dihydroxy-1-benzofuran-3(2H)-one | | | | |
| 47 | (2Z)-4,6-dihydroxy-2-[(2-methyl- | 1.98 | 306.1 | M − H | std method w/ |
| | 1H-indol-3-yl)methylene]-1- | | | | NH₄OAC |
| | benzofuran-3(2H)-one | | | | |
| 48 | (2Z)-4,6-dihydroxy-2-[(6-methyl- | 2.06 | 306.1 | M − H | std method w/ |
| | 1H-indol-3-yl)methylene]-1- | | | | NH₄OAC |
| | benzofuran-3(2H)-one | | | | |
| 49 | (2Z)-4,6-dihydroxy-2-[(7-methyl- | 2.05 | 306.1 | M − H | std method w/ |
| | 1H-indol-3-yl)methylene]-1- | | | | NH₄OAC |
| | benzofuran-3(2H)-one | | | | |
| 50 | (2Z)-2-[(5-bromo-1H-indol-3- | 2.15 | 370 | M − H | std method w/ |
| | yl)methylene]-4,6-dihydroxy-1- | | | | NH₄OAC |
| | benzofuran-3(2H)-one | | | | |
| 51 | (2Z)-2-[(1-benzyl-1H-indol-3- | 2.37 | 382.1 | M − H | std method w/ |
| | yl)methylene]-4,6-dihydroxy-1- | | | | NH₄OAC |
| | benzofuran-3(2H)-one | | | | |
| 52 | 2-{[5-(benzyloxy)-1H-indol-3- | 2.28 | 398.1 | M − H | std method w/ |
| | yl]methylene}-4,6-dihydroxy-1- | | | | NH₄OAC |
| | benzofuran-3(2H)-one | | | | |
| 53 | (2Z)-6,7-dihydroxy-2-[(2-phenyl- | 2.16 | 368.1 | M − H | std method |
| | 1H-indol-3-yl)methylene]-1- | | | | w/NH₄OAC |
| | benzofuran-3(2H)-one | | | | |
| 54 | (2Z)-2-{[2-(4-chlorophenyl)-1H- | 2.28 | 402.1 | M − H | std method |
| | indol-3-yl]methylene}-6,7- | | | | w/NH₄OAC |
| | dihydroxy-1-benzofuran-3(2H)-one | | | | |
| 55 | (2Z)-6,7-dihydroxy-2-{[2-(2- | 2.37 | 420.1 | M + H | std method w/ |
| | naphthyl)-1H-indol-3-yl]methylene}- | | | | formic |
| | 1-benzofuran-3(2H)-one | | | | |
| 56 | (2Z)-2-{[2-(4-fluorophenyl)-1H- | 2.21 | 386.1 | M − H | std method w/ |
| | indol-3-yl]methylene}-6,7- | | | | formic |
| | dihydroxy-1-benzofuran-3(2H)-one | | | | |
| 57 | (2Z)-6,7-dihydroxy-2-[(1-methyl- | 2.14 | 308.1 | M + H | std method w/ |
| | 1H-indol-3-yl)methylene]-1- | | | | formic |
| | benzofuran-3(2H)-one | | | | |
| 58 | (2Z)-2-[(5-bromo-1H-indol-3- | 2.13 | 370 | M − H | std method |
| | yl)methylene]-6,7-dihydroxy-1- | | | | w/NH₄OAC |
| | benzofuran-3(2H)-one | | | | |
| 59 | (2Z)-4,6-dihydroxy-2-[(5-methoxy- | 1.94 | 322.1 | M − H | std method |
| | 1H-indol-3-yl)methylene]-1- | | | | w/formic |
| | benzofuran-3(2H)-one | | | | |
| 60 | (2Z)-2-[(5-chloro-1H-indol-3- | 2.17 | 326 | M − H | std method w/ |
| | yl)methylene]-4,6-dihydroxy-1- | | | | formic |
| | benzofuran-3(2H)-one | | | | |
| 61 | (2Z)-2-[(5-bromo-2-methyl-1H- | 2.24 | 384 | M − H | std method w/ |
| | indol-3-yl)methylene]-4,6- | | | | formic |
| | dihydroxy-1-benzofuran-3(2H)-one | | | | |
| 62 | (2Z)-6,7-dihydroxy-2-[(5-methoxy- | 1.99 | 324.1 | M + H | std method w/ |
| | 1H-indol-3-yl)methylene]-1- | | | | formic |
| | benzofuran-3(2H)-one | | | | |
| 63 | (2Z)-2-[(5-chloro-1H-indol-3- | 2.15 | 328 | M + H | std method w/ |
| | yl)methylene]-6,7-dihydroxy-1- | | | | formic |
| | benzofuran-3(2H)-one | | | | |
| 64 | (2Z)-2-[(5-fluoro-1H-indol-3- | 2.05 | 312.1 | M + H | std method w/ |
| | yl)methylene]-6,7-dihydroxy-1- | | | | formic |
| | benzofuran-3(2H)-one | | | | |
| 65 | (2Z)-6,7-dihydroxy-2-[(5-methyl- | 2.09 | 308.1 | M + H | std method w/ |
| | 1H-indol-3-yl)methylene]-1- | | | | formic |
| | benzofuran-3(2H)-one | | | | |
| 66 | (2Z)-2-[(5-bromo-2-methyl-1H- | 2.13 | 386 | M + H | std method w/ |
| | indol-3-yl)methylene]-6,7- | | | | formic |
| | dihydroxy-1-benzofuran-3(2H)-one | | | | |
| 67 | (2Z)-7-hydroxy-2-[(2-phenyl-1H- | 2.35 | 352.1 | M − H | std method w/ |
| | indol-3-yl)methylene]-1- | | | | formic |
| | benzofuran-3(2H)-one | | | | |
| 68 | (2Z)-2-{[2-(4-fluorophenyl)-1H- | 2.36 | 370.1 | M − H | std method w/ |
| | indol-3-yl]methylene}-7-hydroxy-1- | | | | formic |
| | benzofuran-3(2H)-one | | | | |
| 69 | 6,7-dihydroxy-2-[(5-methoxy-2- | 1.93 | 336.1 | M − H | std method w/ |
| | methyl-1H-indol-3-yl)methylene]-1- | | | | NH₄OAC |
| | benzofuran-3(2H)-one | | | | |
| 70 | 4,6-dihydroxy-2-[(5-methoxy-2- | 2.19 | 398.1 | M − H | std method w/ |
| | phenyl-1H-indol-3-yl)methylene]-1- | | | | NH₄OAC |
| | benzofuran-3(2H)-one | | | | |
| 71 | 6,7-dihydroxy-2-[(5-methoxy-2- | 2.17 | 398.1 | M − H | std method w/ |
| | phenyl-1H-indol-3-yl)methylene]-1- | | | | NH₄OAC |
| | benzofuran-3(2H)-one | | | | |
| 72 | 4,6-dihydroxy-2-[(2-pyridin-2-yl-1H- | 2.08 | 371.1 | M + H | std method |
| | indol-3-yl)methylene]-1- | | | | w/formic |
| | benzofuran-3(2H)-one | | | | |
| 73 | 4,6-dihydroxy-2-[(5-methoxy-2- | 2.01 | 338.1 | M + H | std method w/ |
| | methyl-1H-indol-3-yl)methylene]-1- | | | | formic |
| | benzofuran-3(2H)-one | | | | |
| 74 | 4-hydroxy-2-(1H-indol-3- | 2.12 | 278.1 | M + H | std method w/ |
| | ylmethylene)-1-benzofuran-3(2H)- | | | | formic |
| | one | | | | |
| 75 | 4-hydroxy-2-[(2-phenyl-1H-indol-3- | 2.39 | 354.1 | M + H | std method w/ |
| | yl)methylene]-1-benzofuran-3(2H)- | | | | formic |
| | one | | | | |
| 76 | 2-{[2-(4-chlorophenyl)-1H-indol-3- | 2.46 | 388.1 | M + H | std method w/ |
| | yl]methylene}-4-hydroxy-1- | | | | formic |
| | benzofuran-3(2H)-one | | | | |
| 77 | 2-[(5-bromo-1H-indol-3- | 2.32 | 354 | M − H | std method w/ |
| | yl)methylene]-4-hydroxy-1- | | | | formic |
| | benzofuran-3(2H)-one | | | | |
| 78 | 4-hydroxy-2-[(5-methoxy-1H-indol- | 2.13 | 308.1 | M + H | std method w/ |
| | 3-yl)methylene]-1-benzofuran- | | | | formic |
| | 3(2H)-one | | | | |
| 79 | 4-hydroxy-2-[(5-methoxy-2-phenyl- | | | | |
| | 1H-indol-3-yl)methylene]-1- | | | | |
| | benzofuran-3(2H)-one | | | | |
| 80 | 2-[(2-bromo-1H-indol-3- | 2.1 | 372 | M + H | std method |
| | yl)methylene]-4,6-dihydroxy-1- | | | | w/formic |
| | benzofuran-3(2H)-one | | | | |
| 81 | 2-[(2-bromo-1H-indol-3- | 2.03 | 370 | M − H | std method |
| | yl)methylene]-6,7-dihydroxy-1- | | | | w/formic |
| | benzofuran-3(2H)-one | | | | |
| 84 | 4,6-dihydroxy-2-[(5-methoxy-1- | 2.23 | 338.1 | M + H | std method |
| | methyl-1H-indol-3-yl)methylene]-1- | | | | w/formic |
| | benzofuran-3(2H)-one | | | | |
| 85 | 6,7-dihydroxy-2-[(5-methoxy-1- | 2.19 | 338.1 | M + H | std method |
| | methyl-1H-indol-3-yl)methylene]-1- | | | | w/formic |
| | benzofuran-3(2H)-one | | | | |
| 86 | 4-hydroxy-2-[(5-methoxy-2-methyl- | 2.27 | 322.1 | M + H | std method |
| | 1H-indol-3-yl)methylene]-1- | | | | w/formic |
| | benzofuran-3(2H)-one | | | | |
| 87 | 2-{[1-(4-chlorobutyl)-5-methoxy-2- | 2.28 | 426.1 | M − H | std method |
| | methyl-1H-indol-3-yl]methylene}- | | | | w/formic |
| | 4,6-dihydroxy-1-benzofuran-3(2H)- | | | | |
| | one | | | | |
| 88 | 2-{[1-(3-chloropropyl)-5-methoxy-2- | 2.25 | 414.1 | M + H | std method |
| | methyl-1H-indol-3-yl]methylene}- | | | | w/formic |
| | 4,6-dihydroxy-1-benzofuran-3(2H)- | | | | |
| | one | | | | |
| 89 | 4,6-dihydroxy-2-[(5-methoxy-1,2- | 2.09 | 352.1 | M + H | std method |
| | dimethyl-1H-indol-3-yl)methylene]- | | | | w/formic |
| | 1-benzofuran-3(2H)-one | | | | |
| 90 | 6,7-dihydroxy-2-[(5-methoxy-1,2- | 2.06 | 352.1 | M + H | std method |
| | dimethyl-1H-indol-3-yl)methylene]- | | | | w/formic |
| | 1-benzofuran-3(2H)-one | | | | |
| 91 | 4,6-dihydroxy-2-[(5-methoxy-2- | 2.03 | 399.1 | M − H | std method |
| | pyridin-3-yl-1H-indol-3- | | | | w/formic |
| | yl)methylene]-1-benzofuran-3(2H)- | | | | |
| | one | | | | |
| 92 | 2-{[1-(2-chloroethyl)-2-methyl-1H- | 2.15 | 368.1 | M − H | std method |
| | indol-3-yl]methylene}-4,6- | | | | w/formic |
| | dihydroxy-1-benzofuran-3(2H)-one | | | | |
| 93 | 2-{[1-(3-chloropropyl)-2-methyl-1H- | 2.26 | 382.1 | M − H | std method |
| | indol-3-yl]methylene}-4,6- | | | | w/formic |
| | dihydroxy-1-benzofuran-3(2H)-one | | | | |
| 94 | 2-{[1-(4-chlorobutyl)-2-methyl-1H- | 2.29 | 398.1 | M + H | std method |
| | indol-3-yl]methylene}-4,6- | | | | w/formic |
| | dihydroxy-1-benzofuran-3(2H)-one | | | | |
| 95 | 4,6-dihydroxy-2-({5-methoxy-2- | 1.96 | 478.2 | M + H | std method |
| | methyl-1-[3-(4-methylpiperazin-1- | | | | w/formic |
| | yl)propyl]-1H-indol-3-yl}methylene)- | | | | |
| | 1-benzofuran-3(2H)-one | | | | |
| 96 | 4,6-dihydroxy-2-({5-methoxy-2- | 2 | 492.2 | M + H | std method |
| | methyl-1-[4-(4-methylpiperazin-1- | | | | w/formic |
| | yl)butyl]-1H-indol-3-yl}methylene)- | | | | |
| | 1-benzofuran-3(2H)-one | | | | |
| 97 | 4,6-dihydroxy-2-{[5-methoxy-2- | 1.89 | 479.2 | M + H | std method |
| | methyl-1-(4-morpholin-4-ylbutyl)- | | | | w/formic |
| | 1H-indol-3-yl]methylene}-1- | | | | |
| | benzofuran-3(2H)-one | | | | |
| 98 | 4,6-dihydroxy-2-[(1-{4-[4-(2- | 1.92 | 522.3 | M + H | std method |
| | hydroxyethyl)piperazin-1-yl]butyl}- | | | | w/formic |
| | 5-methoxy-2-methyl-1H-indol-3- | | | | |
| | yl)methylene]-1-benzofuran-3(2H)- | | | | |
| | one | | | | |
| 99 | 2-[(1-{4-[3- | 1.98 | 506.3 | M + H | std method |
| | (dimethylamino)pyrrolidin-1- | | | | w/formic |
| | yl]butyl}-5-methoxy-2-methyl-1H- | | | | |
| | indol-3-yl)methylene]-4,6- | | | | |
| | dihydroxy-1-benzofuran-3(2H)-one | | | | |
| 100 | 4,6-dihydroxy-2-[(5-methoxy-2- | 1.89 | 591.3 | M + H | std method |
| | methyl-1-{4-[4-(2-morpholin-4- | | | | w/formic |
| | ylethyl)piperazin-1-yl]butyl}-1H- | | | | |
| | indol-3-yl)methylene]-1- | | | | |
| | benzofuran-3(2H)-one | | | | |
| 101 | 2-({1-[4-(dimethylamino)butyl]-5- | 1.9 | 437.2 | M + H | std method |
| | methoxy-2-methyl-1H-indol-3- | | | | w/formic |
| | yl}methylene)-4,6-dihydroxy-1- | | | | |
| | benzofuran-3(2H)-one | | | | |
| 102 | 4,6-dihydroxy-2-{[5-methoxy-2- | 1.88 | 465.2 | M + H | std method |
| | methyl-1-(3-morpholin-4-ylpropyl)- | | | | w/formic |
| | 1H-indol-3-yl]methylene}-1- | | | | |
| | benzofuran-3(2H)-one | | | | |
| 103 | 4,6-dihydroxy-2-[(1-{3-[4-(2- | 1.9 | 508.2 | M + H | std method |
| | hydroxyethyl)piperazin-1-yl]propyl}- | | | | w/formic |
| | 5-methoxy-2-methyl-1H-indol-3- | | | | |
| | yl)methylene]-1-benzofuran-3(2H)- | | | | |
| | one | | | | |
| 104 | 2-[(1-{3-[3- | 1.96 | 492.2 | M + H | std method |
| | (dimethylamino)pyrrolidin-1- | | | | w/formic |
| | yl]propyl}-5-methoxy-2-methyl-1H- | | | | |
| | indol-3-yl)methylene]-4,6- | | | | |
| | dihydroxy-1-benzofuran-3(2H)-one | | | | |
| 105 | 4,6-dihydroxy-2-[(5-methoxy-2- | 1.86 | 577.3 | M + H | std method |
| | methyl-1-{3-[4-(2-morpholin-4- | | | | w/formic |
| | ylethyl)piperazin-1-yl]propyl}-1H- | | | | |
| | indol-3-yl)methylene]-1- | | | | |
| | benzofuran-3(2H)-one | | | | |
| 106 | 2-({1-[3-(dimethylamino)propyl]-5- | 1.86 | 423.2 | M + H | std method |
| | methoxy-2-methyl-1H-indol-3- | | | | w/formic |
| | yl}methylene)-4,6-dihydroxy-1- | | | | |
| | benzofuran-3(2H)-one | | | | |
| 107 | 4,6-dihydroxy-2-{[5-methoxy-2- | 1.96 | 451.2 | M + H | std method |
| | methyl-1-(2-morpholin-4-ylethyl)- | | | | w/formic |
| | 1H-indol-3-yl]methylene}-1- | | | | |
| | benzofuran-3(2H)-one | | | | |
| 108 | 4,6-dihydroxy-2-({5-methoxy-2- | 1.89 | 464.2 | M + H | std method |
| | methyl-1-[2-(4-methylpiperazin-1- | | | | w/formic |
| | yl)ethyl]-1H-indol-3-yl}methylene)- | | | | |
| | 1-benzofuran-3(2H)-one | | | | |
| 109 | 4,6-dihydroxy-2-[(1-{2-[4-(2- | 1.86 | 494.2 | M + H | std method |
| | hydroxyethyl)piperazin-1-yl]ethyl}- | | | | w/formic |
| | 5-methoxy-2-methyl-1H-indol-3- | | | | |
| | yl)methylene]-1-benzofuran-3(2H)- | | | | |
| | one | | | | |
| 110 | 2-({1-[2-(dimethylamino)ethyl]-5- | 1.82 | 409.2 | M + H | std method |
| | methoxy-2-methyl-1H-indol-3- | | | | w/formic |
| | yl}methylene)-4,6-dihydroxy-1- | | | | |
| | benzofuran-3(2H)-one | | | | |
| 111 | 4,6-dihydroxy-2-[(1-{2-[4-(2- | 1.82 | 480.2 | M + H | std method |
| | hydroxyethyl)piperazin-1-yl]ethyl}- | | | | w/formic |
| | 5-methoxy-1H-indol-3- | | | | |
| | yl)methylene]-1-benzofuran-3(2H)- | | | | |
| | one | | | | |
| 112 | 4,6-dihydroxy-2-{[5-methoxy-1-(3- | 1.83 | 451.2 | M + H | std method |
| | morpholin-4-ylpropyl)-1H-indol-3- | | | | w/formic |
| | yl]methylene}-1-benzofuran-3(2H)- | | | | |
| | one | | | | |
| 113 | 4,6-dihydroxy-2-[(1-{3-[4-(2- | 1.85 | 494.2 | M + H | std method |
| | hydroxyethyl)piperazin-1-yl]propyl}- | | | | w/formic |
| | 5-methoxy-1H-indol-3- | | | | |
| | yl)methylene]-1-benzofuran-3(2H)- | | | | |
| | one | | | | |
| 114 | 2-({1-[3-(dimethylamino)propyl]-5- | 1.8 | 409.2 | M + H | std method |
| | methoxy-1H-indol-3-yl}methylene)- | | | | w/formic |
| | 4,6-dihydroxy-1-benzofuran-3(2H)- | | | | |
| | one | | | | |
| 115 | 4,6-dihydroxy-2-{[5-methoxy-1-(4- | 1.85 | 465.2 | M + H | std method |
| | morpholin-4-ylbutyl)-1H-indol-3- | | | | w/formic |
| | yl]methylene}-1-benzofuran-3(2H)- | | | | |
| | one | | | | |
| 116 | 2-({1-[4-(dimethylamino)butyl]-5- | 1.84 | 423.2 | M + H | std method |
| | methoxy-1H-indol-3-yl}methylene)- | | | | w/formic |
| | 4,6-dihydroxy-1-benzofuran-3(2H)- | | | | |
| | one | | | | |
| 120 | 7-hydroxy-2-[(5-methoxy-2-methyl- | 1.97 | 322.1 | M + H | std method |
| | 1H-indol-3-yl)methylene]-1- | | | | w/formic |
| | benzofuran-3(2H)-one | | | | |
| 121 | 4-hydroxy-2-[(5-methoxy-1,2- | 2.29 | 336.1 | M + H | std method |
| | dimethyl-1H-indol-3-yl)methylene]- | | | | w/formic |
| | 1-benzofuran-3(2H)-one | | | | |
| 122 | 2-{[1-(4-chlorobutyl)-5-methoxy-2- | 2.25 | 428.1 | M + H | std method |
| | methyl-1H-indol-3-yl]methylene}- | | | | w/formic |
| | 6,7-dihydroxyl-1-benzofuran-3(2H)- | | | | |
| | one | | | | |
| 123 | 2-{[1-(4-chlorobutyl)-5-methoxy-2- | 2.44 | 412.1 | M + H | std method |
| | methyl-1H-indol-3-yl]methylene}-4- | | | | w/formic |
| | hydroxy-1-benzofuran-3(2H)-one | | | | |
| 124 | 2-{[1-(3-chloropropyl)-5-methoxy-2- | 2.21 | 414.1 | M + H | std method |
| | methyl-1H-indol-3-yl]methylene}- | | | | w/formic |
| | 6,7-dihydroxy-1-benzofuran-3(2H)- | | | | |
| | one | | | | |
| 125 | 2-{[1-(3-chloropropyl)-5-methoxy-2- | 2.41 | 398.1 | M + H | std method |
| | methyl-1H-indol-3-yl]methylene}-4- | | | | w/formic |
| | hydroxy-1-benzofuran-3(2H)-one | | | | |
| 129 | 4-hydroxy-2-({5-methoxy-2-methyl- | 1.92 | 478.2 | M + H | std method |
| | 1-[2-(4-methylpiperazin-1-yl)ethyl]- | | | | w/formic |
| | 1H-indol-3-yl}methylene)-1- | | | | |
| | benzofuran-3(2H)-one | | | | |
| 130 | 4-hydroxy-2-{[5-methoxy-2-methyl- | 1.85 | 465.2 | M + H | std method |
| | 1-(2-morpholin-4-ylethyl)-1H-indol- | | | | w/formic |
| | 3-yl]methylene}-1-benzofuran- | | | | |
| | 3(2H)-one | | | | |
| 131 | 4-hydroxy-2-[(1-{2-[4-(2- | 1.86 | 508.2 | M + H | std method |
| | hydroxyethyl)piperazin-1-yl]ethyl}- | | | | w/formic |
| | 5-methoxy-2-methyl-1H-indol-3- | | | | |
| | yl)methylene]-1-benzofuran-3(2H)- | | | | |
| | one | | | | |
| 132 | 6,7-dihydroxy-2-({5-methoxy-2- | 2.11 | 462.2 | M + H | std method |
| | methyl-1-[4-(4-methylpiperazin-1- | | | | w/formic |
| | yl)butyl]-1H-indol-3-yl}methylene)- | | | | |
| | 1-benzofuran-3(2H)-one | | | | |
| 133 | 6,7-dihydroxy-2-{[5-methoxy-2- | 2 | 449.2 | M + H | std method |
| | methyl-1-(4-morpholin-4-ylbutyl)- | | | | w/formic |
| | 1H-indol-3-yl]methylene}-1- | | | | |
| | benzofuran-3(2H)-one | | | | |
| 134 | 6,7-dihydroxy-2-[(1-{4-[4-(2- | 2.02 | 492.2 | M + H | std method |
| | hydroxyethyl)piperazin-1-yl]butyl}- | | | | w/formic |
| | 5-methoxy-2-methyl-1H-indol-3- | | | | |
| | yl)methylene]-1-benzofuran-3(2H)- | | | | |
| | one | | | | |
| 135 | 4-hydroxy-2-({5-methoxy-2-methyl- | 2.17 | 476.2 | M + H | std method |
| | 1-[4-(4-methylpiperazin-1-yl)butyl]- | | | | w/formic |
| | 1H-indol-3-yl}methylene)-1- | | | | |
| | benzofuran-3(2H)-one | | | | |
| 136 | 4-hydroxy-2-{[5-methoxy-2-methyl- | 2.02 | 463.2 | M + H | std method |
| | 1-(4-morpholin-4-ylbutyl)-1H-indol- | | | | w/formic |
| | 3-yl]methylene}-1-benzofuran- | | | | |
| | 3(2H)-one | | | | |
| 137 | 4-hydroxy-2-[(1-{4-[4-(2- | 2.06 | 506.3 | M + H | std method |
| | hydroxyethyl)piperazin-1-yl]butyl}- | | | | w/formic |
| | 5-methoxy-2-methyl-1H-indol-3- | | | | |
| | yl)methylene]-1-benzofuran-3(2H)- | | | | |
| | one | | | | |
| 143 | 2-[(6-bromo-1H-indol-3- | 2.12 | 370 | M − H | std method |
| | yl)methylene]-4,6-dihydroxy-1- | | | | w/formic |
| | benzofuran-3(2H)-one | | | | |
| 144 | 6,7-dihydroxy-2-({5-methoxy-2- | 1.96 | 492.2 | M + H | std method |
| | methyl-1-[4-(4-methylpiperazin-1- | | | | w/formic |
| | yl)butyl]-1H-indol-3-yl}methylene)- | | | | |
| | 1-benzofuran-3(2H)-one | | | | |
| 145 | 6,7-dihydroxy-2-{[5-methoxy-2- | | | | |
| | methyl-1-(4-morpholin-4-ylbutyl)- | | | | |
| | 1H-indol-3-yl]methylene}-1- | | | | |
| | benzofuran-3(2H)-one | | | | |
| 146 | 6,7-dihydroxy-2-[(1-{4-[4-(2- | 1.88 | 522.3 | M + H | std method |
| | hydroxyethyl)piperazin-1-yl]butyl}- | | | | w/formic |
| | 5-methoxy-2-methyl-1H-indol-3- | | | | |
| | yl)methylene]-1-benzofuran-3(2H)- | | | | |
| | one | | | | |
| 147 | 4,6-dihydroxy-2-{[1-(2-morpholin-4- | 2.09 | 483.2 | M + H | std method |
| | ylethyl)-2-phenyl-1H-indol-3- | | | | w/formic |
| | yl]methylene}-1-benzofuran-3(2H)- | | | | |
| | one | | | | |
| 148 | 2-({1-[2-(dimethylamino)ethyl]-2- | 1.89 | 441.2 | M + H | std method |
| | phenyl-1H-indol-3-yl}methylene)- | | | | w/formic |
| | 4,6-dihydroxy-1-benzofuran-3(2H)- | | | | |
| | one | | | | |
| 149 | 2-[(1-benzyl-2-phenyl-1H-indol-3- | 2.43 | 460.1 | M + H | std method |
| | yl)methylene]-4,6-dihydroxy-1- | | | | w/formic |
| | benzofuran-3(2H)-one | | | | |
| 150 | 4,6-dihydroxy-2-[(1-isobutyl-2- | 2.46 | 426.2 | M + H | std method |
| | phenyl-1H-indol-3-yl)methylene]-1- | | | | w/formic |
| | benzofuran-3(2H)-one | | | | |
| 151 | 4,6-dihydroxy-2-{[1-(2- | 2.25 | 428.1 | M + H | std method |
| | methoxyethyl)-2-phenyl-1H-indol-3- | | | | w/formic |
| | yl]methylene}-1-benzofuran-3(2H)- | | | | |
| | one | | | | |
| 152 | 2-{[1-(cyclopropylmethyl)-2-phenyl- | 2.4 | 424.1 | M + H | std method |
| | 1H-indol-3-yl]methylene}-4,6- | | | | w/formic |
| | dihydroxy-1-benzofuran-3(2H)-one | | | | |
| 153 | 4,6-dihydroxy-2-{[2-phenyl-1- | 2.2 | 461.1 | M + H | std method |
| | (pyridin-3-ylmethyl)-1H-indol-3- | | | | w/formic |
| | yl]methylene}-1-benzofuran-3(2H)- | | | | |
| | one | | | | |
| 154 | 4,6-dihydroxy-2-{[2-phenyl-1- | 2.19 | 461.1 | M + H | std method |
| | (pyridin-4-ylmethyl)-1H-indol-3- | | | | w/formic |
| | yl]methylene}-1-benzofuran-3(2H)- | | | | |
| | one | | | | |
| 155 | 4-{3-[(4,6-dihydroxy-3-oxo-1- | 2.29 | 437.1 | M + H | std method |
| | benzofuran-2(3H)-ylidene)methyl]- | | | | w/formic |
| | 2-phenyl-1H-indol-1-yl}butanenitrile | | | | |
| 156 | 2-({1-[3-(dimethylamino)propyl]-2- | 1.95 | 455.2 | M + H | std method |
| | phenyl-1H-indol-3-yl}methylene)- | | | | w/formic |
| | 4,6-dihydroxy-1-benzofuran-3(2H)- | | | | |
| | one | | | | |
| 157 | 4,6-dihydroxy-2-{[2-phenyl-1-(2- | 1.94 | 467.2 | M + H | std method |
| | pyrrolidin-1-ylethyl)-1H-indol-3- | | | | w/formic |
| | yl]methylene}-1-benzofuran-3(2H)- | | | | |
| | one | | | | |
| 158 | 4,6-dihydroxy-2-{[2-phenyl-1-(2- | 1.95 | 481.2 | M + H | std method |
| | piperidin-4-ylethyl)-1H-indol-3- | | | | w/formic |
| | yl]methylene}-1-benzofuran-3(2H)- | | | | |
| | one | | | | |
| 159 | 4,6-dihydroxy-2-({1-[2-(4- | 2.01 | 496.2 | M + H | std method |
| | methylpiperazin-1-yl)ethyl]-2- | | | | w/formic |
| | phenyl-1H-indol-3-yl}methylene)-1- | | | | |
| | benzofuran-3(2H)-one | | | | |
| 160 | 4,6-dihydroxy-2-{[2-phenyl-1-(2- | 1.95 | 482.2 | M + H | std method |
| | piperazin-1-ylethyl)-1H-indol-3- | | | | w/formic |
| | yl]methylene}-1-benzofuran-3(2H)- | | | | |
| | one | | | | |
| 161 | 2-{3-[(4,6-dihydroxy-3-oxo-1- | | | | |
| | benzofuran-2(3H)-ylidene)methyl]- | | | | |
| | 2-phenyl-1H-indol-1-yl}acetamide | | | | |
| 162 | 4,6-dihydroxy-2-[(2-methyl-5-nitro- | 2.2 | 353.1 | M + H | std method |
| | 1H-indol-3-yl)methylene]-1- | | | | w/formic@280 nm |
| | benzofuran-3(2H)-one | | | | |
| 164 | 4,6-dihydroxy-2-[(5-hydroxy-1H- | 1.85 | 310.1 | M + H | std method |
| | indol-3-yl)methylene]-1- | | | | w/formic |
| | benzofuran-3(2H)-one | | | | |
| 165 | 3-[(4,6-dihydroxy-3-oxo-1- | 1.91 | 338.1 | M + H | std method |
| | benzofuran-2(3H)-ylidene)methyl]- | | | | w/formic |
| | 1H-indole-5-carboxylic acid | | | | |
| 166 | methyl 3-[(4,6-dihydroxy-3-oxo-1- | 2.1 | 352.1 | M + H | std method |
| | benzofuran-2(3H)-ylidene)methyl]- | | | | w/formic |
| | 1H-indole-5-carboxylate | | | | |
| 167 | 3-[(4,6-dihydroxy-3-oxo-1- | 2.11 | 317.1 | M − H | std method |
| | benzofuran-2(3H)-ylidene)methyl]- | | | | w/formic |
| | 1H-indole-6-carbonitrile | | | | |
| 168 | 2-(5H-[1,3]dioxolo[4,5-f]indol-7- | 2.07 | 338.1 | M + H | std method |
| | ylmethylene)-4,6-dihydroxy-1- | | | | w/formic@300 nm |
| | benzofuran-3(2H)-one | | | | |
| 169 | 4,6-dihydroxy-2-{[6- | 1.93 | 372 | M + H | std method |
| | (methylsulfonyl)-1H-indol-3- | | | | w/formic |
| | yl]methylene}-1-benzofuran-3(2H)- | | | | |
| | one | | | | |
| 170 | 4,6-dihydroxy-2-[(5-methyl-1H- | 2.17 | 308.1 | M + H | std method |
| | indol-3-yl)methylene]-1- | | | | w/formic |
| | benzofuran-3(2H)-one | | | | |
| 171 | 2-[(4-chloro-1H-indol-3- | 2.21 | 328 | M + H | std method |
| | yl)methylene]-4,6-dihydroxy-1- | | | | w/formic |
| | benzofuran-3(2H)-one | | | | |
| 172 | 2-[(6-chloro-1H-indol-3- | 2.24 | 328 | M + H | std method |
| | yl)methylene]-4,6-dihydroxy-1- | | | | w/formic |
| | benzofuran-3(2H)-one | | | | |
| 173 | 2-[(7-chloro-1H-indol-3- | 2.24 | 326 | M − H | std method |
| | yl)methylene]-4,6-dihydroxy-1- | | | | w/NH₄OAc |
| | benzofuran-3(2H)-one | | | | |
| 174 | 2-[(4-bromo-1H-indol-3- | 2.23 | 372 | M + H | std method |
| | yl)methylene]-4,6-dihydroxy-1- | | | | w/formic |
| | benzofuran-3(2H)-one | | | | |
| 175 | 2-[(5-fluoro-1H-indol-3- | 2.14 | 312.1 | M + H | std method |
| | yl)methylene]-4,6-dihydroxy-1- | | | | w/formic |
| | benzofuran-3(2H)-one | | | | |
| 176 | 2-[(6-fluoro-1H-indol-3- | 2.13 | 310.1 | M − H | std method |
| | yl)methylene]-4,6-dihydroxy-1- | | | | w/formic |
| | benzofuran-3(2H)-one | | | | |
| 177 | 4,6-dihydroxy-2-[(5-iodo-1H-indol- | 2.3 | 420 | M + H | std method |
| | 3-yl)methylene]-1-benzofuran- | | | | w/formic |
| | 3(2H)-one | | | | |
| 178 | 4,6-dihydroxy-2-[(5-nitro-1H-indol- | 2.15 | 337.1 | M − H | std method |
| | 3-yl)methylene]-1-benzofuran- | | | | w/formic |
| | 3(2H)-one | | | | |
| 179 | 4,6-dihydroxy-2-[(6-nitro-1H-indol- | 2.18 | 337.1 | M − H | std method |
| | 3-yl)methylene]-1-benzofuran- | | | | w/formic |
| | 3(2H)-one | | | | |
| 180 | 4,6-dihydroxy-2-[(7-nitro-1H-indol- | 2.26 | 337.1 | M − H | std method |
| | 3-yl)methylene]-1-benzofuran- | | | | w/formic |
| | 3(2H)-one | | | | |
| 181 | 2-[(5,6-dimethoxy-1H-indol-3- | | | | |
| | yl)methylene]-4,6-dihydroxy-1- | | | | |
| | benzofuran-3(2H)-one | | | | |
| 182 | 3-[(4,6-dihydroxy-3-oxo-1- | 2.09 | 317.1 | M − H | std method |
| | benzofuran-2(3H)-ylidene)methyl]- | | | | w/formic |
| | 1H-indole-5-carbonitrile | | | | |
| 183 | N-{3-[(4,6-dihydroxy-3-oxo-1- | 2.03 | 403.1 | M + H | std method |
| | benzofuran-2(3H)-ylidene)methyl]- | | | | w/formic |
| | 1H-indol-5-yl}-2-furamide | | | | |
| 184 | 4,6-dihydroxy-2-[(5-methoxy-2,6- | 2.22 | 352.1 | M + H | std method |
| | dimethyl-1H-indol-3-yl)methylene]- | | | | w/formic |
| | 1-benzofuran-3(2H)-one | | | | |
| 185 | 4,6-dihydroxy-2-[(5-methoxy-1,2,6- | 2.38 | 366.1 | M + H | std method |
| | trimethyl-1H-indol-3-yl)methylene]- | | | | w/formic |
| | 1-benzofuran-3(2H)-one | | | | |
| 186 | 4,6-dihydroxy-2-[(6-methoxy-1H- | 2.07 | 324.1 | M + H | std method |
| | indol-3-yl)methylene]-1- | | | | w/formic |
| | benzofuran-3(2H)-one | | | | |
| 187 | 2-[(7-ethyl-1H-indol-3- | 2.25 | 322.1 | M + H | std method |
| | yl)methylene]-4,6-dihydroxy-1- | | | | w/formic |
| | benzofuran-3(2H)-one | | | | |
| 188 | 3-[(4,6-dihydroxy-3-oxo-1- | 2.26 | 333.1 | M + H | std method |
| | benzofuran-2(3H)-ylidene)methyl]- | | | | w/formic |
| | 1-methyl-1H-indole-4-carbonitrile | | | | |
| 189 | 4,6-dihydroxy-2-[(1-methyl-2- | 2.16 | 385.1 | M + H | std method |
| | pyridin-3-yl-1H-indol-3- | | | | w/formic |
| | yl)methylene]-1-benzofuran-3(2H)- | | | | |
| | one | | | | |
| 190 | 4,6-dihydroxy-2-[(1-methyl-2- | 2.06 | 385.1 | M + H | std method |
| | pyridin-4-y1-1H-indol-3- | | | | w/formic |
| | yl)methylene]-1-benzofuran-3(2H)- | | | | |
| | one | | | | |
| 191 | 2-{[2-(3,5-dimethylisoxazol-4-yl)-1- | 2.27 | 403.1 | M + H | std method |
| | methyl-1H-indol-3-yl]methylene}- | | | | w/formic |
| | 4,6-dihydroxy-1-benzofuran-3(2H)- | | | | |
| | one | | | | |
| 192 | 4,6-dihydroxy-2-{[2-(3- | 2.29 | 400.1 | M + H | std method |
| | hydroxyphenyl)-1-methyl-1H-indol- | | | | w/formic |
| | 3-yl]methylene}-1-benzofuran- | | | | |
| | 3(2H)-one | | | | |
| 193 | 4,6-dihydroxy-2-{[2-(4- | 2.28 | 400.1 | M + H | std method |
| | hydroxyphenyl)-1-methyl-1H-indol- | | | | w/formic |
| | 3-yl]methylene}-1-benzofuran- | | | | |
| | 3(2H)-one | | | | |
| 194 | 4,6-dihydroxy-2-{[1-methyl-2-(3- | 2.42 | 390.1 | M + H | std method |
| | thienyl)-1H-indol-3-yl]methylene}-1- | | | | w/formic |
| | benzofuran-3(2H)-one | | | | |
| 195 | 4,6-dihydroxy-2-{[2-(4- | 2.44 | 414.1 | M + H | std method |
| | methoxyphenyl)-1-methyl-1H-indol- | | | | w/formic |
| | 3-yl]methylene}-1-benzofuran- | | | | |
| | 3(2H)-one | | | | |
| 196 | 4,6-dihydroxy-2-{[2-(3- | 2.47 | 414.1 | M + H | std method |
| | methoxyphenyl)-1-methyl-1H-indol- | | | | w/formic |
| | 3-yl]methylene}-1-benzofuran- | | | | |
| | 3(2H)-one | | | | |
| 197 | 2-{[2-(3-fluorophenyl)-1-methyl-1H- | 2.46 | 402.1 | M + H | std method |
| | indol-3-yl]methylene}-4,6- | | | | w/formic |
| | dihydroxy-1-benzofuran-3(2H)-one | | | | |
| 198 | 2-({2-[4-(dimethylamino)phenyl]-1- | 2.64 | 427.2 | M + H | std method |
| | methyl-1H-indol-3-yl}methylene)- | | | | w/formic |
| | 4,6-dihydroxy-1-benzofuran-3(2H)- | | | | |
| | one | | | | |
| 199 | 2-{[2-(3-chloro-4-fluorophenyl)-1- | 2.53 | 436.1 | M + H | std method |
| | methyl-1H-indol-3-yl]methylene}- | | | | w/formic |
| | 4,6-dihydroxy-1-benzofuran-3(2H)- | | | | |
| | one | | | | |
| 200 | 2-{[2-(4-fluorophenyl)-1-methyl-1H- | 2.46 | 402.1 | M + H | std method |
| | indol-3-yl]methylene}-4,6- | | | | w/formic |
| | dihydroxy-1-benzofuran-3(2H)-one | | | | |
| 201 | 2-{[2-(4-chlorophenyl)-1-methyl-1H- | 2.54 | 418.1 | M + H | std method |
| | indol-3-yl]methylene}-4,6- | | | | w/formic |
| | dihydroxy-1-benzofuran-3(2H)-one | | | | |
| 202 | 4,6-dihydroxy-2-[(2-pyridin-3-yl-1H- | | | | |
| | indol-3-yl)methylene]-1- | | | | |
| | benzofuran-3(2H)-one | | | | |
| 203 | 4,6-dihydroxy-2-[(2-pyridin-4-yl-1H- | 1.88 | 371.1 | M + H | std method |
| | indol-3-yl)methylene]-1- | | | | w/formic |
| | benzofuran-3(2H)-one | | | | |
| 204 | 2-{[2-(3,5-dimethylisoxazol-4-yl)- | | | | |
| | 1H-indol-3-yl]methylene}-4,6- | | | | |
| | dihydroxy-1-benzofuran-3(2H)-one | | | | |
| 205 | 4,6-dihydroxy-2-{[2-(3- | 2.18 | 386.1 | M + H | std method |
| | hydroxyphenyl)-1H-indol-3- | | | | w/formic |
| | yl]methylene}-1-benzofuran-3(2H)- | | | | |
| | one | | | | |
| 206 | 4,6-dihydroxy-2-{[2-(4- | 2.16 | 386.1 | M + H | std method |
| | hydroxyphenyl)-1H-indol-3- | | | | w/formic |
| | yl]methylene}-1-benzofuran-3(2H)- | | | | |
| | one | | | | |
| 207 | 4,6-dihydroxy-2-{[2-(3-thienyl)-1H- | 2.3 | 374.1 | M − H | std method |
| | indol-3-yl]methylene}-1- | | | | w/formic |
| | benzofuran-3(2H)-one | | | | |
| 208 | 4,6-dihydroxy-2-{[2-(4- | 2.33 | 400.1 | M + H | std method |
| | methoxyphenyl)-1H-indol-3- | | | | w/formic |
| | yl]methylene}-1-benzofuran-3(2H)- | | | | |
| | one | | | | |
| 209 | 4,6-dihydroxy-2-{[2-(3- | 2.34 | 400.1 | M + H | std method |
| | methoxyphenyl)-1H-indol-3- | | | | w/formic |
| | yl]methylene}-1-benzofuran-3(2H)- | | | | |
| | one | | | | |
| 210 | 2-{[2-(3-fluorophenyl)-1H-indol-3- | 2.35 | 386.1 | M − H | std method |
| | yl]methylene}-4,6-dihydroxy-1- | | | | w/formic |
| | benzofuran-3(2H)-one | | | | |
| 211 | 2-({2-[4-(dimethylamino)phenyl]- | 2.32 | 413.1 | M + H | std method |
| | 1H-indol-3-yl}methylene)-4,6- | | | | w/formic |
| | dihydroxy-1-benzofuran-3(2H)-one | | | | |
| 212 | 2-{[2-(3-chloro-4-fluorophenyl)-1H- | 2.43 | 422.1 | M + H | std method |
| | indol-3-yl]methylene}-4,6- | | | | w/formic |
| | dihydroxy-1-benzofuran-3(2H)-one | | | | |
| 213 | 2-[(1-ethyl-2-phenyl-1H-indol-3- | 2.52 | 398.1 | M + H | std method |
| | yl)methylene]-4,6-dihydroxy-1- | | | | w/formic |
| | benzofuran-3(2H)-one | | | | |
| 214 | 4,6-dihydroxy-2-[(2-phenyl-1- | 2.59 | 412.1 | M + H | std method |
| | propyl-1H-indol-3-yl)methylene]-1- | | | | w/formic |
| | benzofuran-3(2H)-one | | | | |
| 215 | 2-[(5-chloro-2-methyl-1H-indol-3- | 2.22 | 342 | M + H | std method |
| | yl)methylene]-4,6-dihydroxy-1- | | | | w/formic |
| | benzofuran-3(2H)-one | | | | |
| 216 | N-{3-[(4,6-dihydroxy-3-oxo-1- | 1.86 | 365.1 | M + H | std method |
| | benzofuran-2(3H)-ylidene)methyl]- | | | | w/formic |
| | 2-methyl-1H-indol-5-yl}acetamide | | | | |
| 217 | 2-[(7-bromo-2-methyl-1H-indol-3- | 2.26 | 386 | M + H | std method |
| | yl)methylene]-4,6-dihydroxy-1- | | | | w/formic |
| | benzofuran-3(2H)-one | | | | |
| 218 | 2-[(5-fluoro-2-methyl-1H-indol-3- | 2.12 | 326.1 | M + H | std method |
| | yl)methylene]-4,6-dihydroxy-1- | | | | w/formic |
| | benzofuran-3(2H)-one | | | | |
| 219 | 4,6-dihydroxy-2-[(5-methoxy-4- | 2.1 | 338.1 | M + H | std method |
| | methyl-1H-indol-3-yl)methylene]-1- | | | | w/formic |
| | benzofuran-3(2H)-one | | | | |
| 220 | 3-[(4,6-dihydroxy-3-oxo-1- | 2.1 | 317.1 | M − H | std method |
| | benzofuran-2(3H)-ylidene)methyl]- | | | | w/formic |
| | 1H-indole-4-carbonitrile | | | | |
| 221 | 4,6-dihydroxy-2-{[6- | 2.25 | 360.1 | M − H | std method |
| | (trifluoromethyl)-1H-indol-3- | | | | w/formic |
| | yl]methylene}-1-benzofuran-3(2H)- | | | | |
| | one | | | | |
| 222 | methyl 3-[(4,6-dihydroxy-3-oxo-1- | 2.06 | 352.1 | M + H | std method |
| | benzofuran-2(3H)-ylidene)methyl]- | | | | w/formic |
| | 1H-indole-4-carboxylate | | | | |
| 223 | 4,6-dihydroxy-2-[(1-methyl-2- | 2.25 | 385.1 | M + H | std method |
| | pyridin-2-yl-1H-indol-3- | | | | w/formic |
| | yl)methylene]-1-benzofuran-3(2H)- | | | | |
| | one | | | | |
| 224 | 5-{3-[(4,6-dihydroxy-3-oxo-1- | 2.47 | 451.2 | M + H | std method |
| | benzofuran-2(3H)-ylidene)methyl]- | | | | w/formic |
| | 2-phenyl-1H-indol-1- | | | | |
| | yl}pentanenitrile | | | | |
| 225 | 6-{3-[(4,6-dihydroxy-3-oxo-1- | 2.54 | 465.2 | M + H | std method |
| | benzofuran-2(3H)-ylidene)methyl]- | | | | w/formic |
| | 2-phenyl-1H-indol-1- | | | | |
| | yl}hexanenitrile | | | | |
| 226 | 4-{3-[(4,6-dihydroxy-3-oxo-1- | 2.13 | 438.1 | M + H | std method |
| | benzofuran-2(3H)-ylidene)methyl]- | | | | w/formic |
| | 2-pyridin-3-yl-1H-indol-1- | | | | |
| | yl}butanenitrile | | | | |
| 227 | 2-[(5-fluoro-1-methyl-1H-indol-3- | 2.29 | 326.1 | M + H | std method |
| | yl)methylene]-4,6-dihydroxy-1- | | | | w/formic |
| | benzofuran-3(2H)-one | | | | |
| 228 | 4,6-dihydroxy-2-[(1-methyl-5-nitro- | 2.28 | 353.1 | M + H | std method |
| | 1H-indol-3-yl)methylene]-1- | | | | w/formic |
| | benzofuran-3(2H)-one | | | | |
| 229 | 4,6-dihydroxy-2-[(1-methyl-7-nitro- | 2.33 | 351.1 | M − H | std method |
| | 1H-indol-3-yl)methylene]-1- | | | | w/formic |
| | benzofuran-3(2H)-one | | | | |
| 230 | 4-{4-bromo-3-[(4,6-dihydroxy-3- | 2.34 | 437 | M + H | std method |
| | oxo-1-benzofuran-2(3H)- | | | | w/formic |
| | ylidene)methyl]-1H-indol-1- | | | | |
| | yl}butanenitrile | | | | |
| 231 | 4-{3-[(4,6-dihydroxy-3-oxo-1- | 2.24 | 379.1 | M + H | std method |
| | benzofuran-2(3H)-ylidene)methyl]- | | | | w/formic |
| | 5-fluoro-1H-indol-1-yl}butanenitrile | | | | |
| 232 | 4-{3-[(4,6-dihydroxy-3-oxo-1- | 2.36 | 389.1 | M + H | std method |
| | benzofuran-2(3H)-ylidene)methyl]- | | | | w/formic |
| | 7-ethyl-1H-indol-1-yl}butanenitrile | | | | |
| 233 | 2-[(5-chloro-1,2-dimethyl-1H-indol- | 2.4 | 356.1 | M + H | std method |
| | 3-yl)methylene]-4,6-dihydroxy-1- | | | | w/formic |
| | benzofuran-3(2H)-one | | | | |
| 234 | 2-[(7-bromo-1,2-dimethyl-1H-indol- | 2.48 | 400 | M + H | std method |
| | 3-yl)methylene]-4,6-dihydroxy-1- | | | | w/formic |
| | benzofuran-3(2H)-one | | | | |
| 235 | 2-[(5-fluoro-1,2-dimethyl-1H-indol- | 2.29 | 340.1 | M + H | std method |
| | 3-yl)methylene]-4,6-dihydroxy-1- | | | | w/formic@300 nm |
| | benzofuran-3(2H)-one | | | | |
| 236 | 4,6-dihydroxy-2-[(5-methoxy-1,4- | 2.29 | 352.1 | M + H | std method |
| | dimethyl-1H-indol-3-yl)methylene]- | | | | w/formic |
| | 1-benzofuran-3(2H)-one | | | | |
| 237 | 4,6-dihydroxy-2-{[1-methyl-6- | 2.41 | 374.1 | M − H | std method |
| | (trifluoromethyl)-1H-indol-3- | | | | w/formic |
| | yl]methylene}-1-benzofuran-3(2H)- | | | | |
| | one | | | | |
| 238 | 4-{5-chloro-3-[(4,6-dihydroxy-3- | 2.33 | 409.1 | M + H | std method |
| | oxo-1-benzofuran-2(3H)- | | | | w/formic |
| | ylidene)methyl]-2-methyl-1H-indol- | | | | |
| | 1-yl}butanenitrile | | | | |
| 239 | 4-{3-[(4,6-dihydroxy-3-oxo-1- | 2.24 | 405.1 | M + H | std method |
| | benzofuran-2(3H)-ylidene)methyl]- | | | | w/formic |
| | 5-methoxy-4-methyl-1H-indol-1- | | | | |
| | yl}butanenitrile | | | | |
| 240 | 4-{3-[(4,6-dihydroxy-3-oxo-1- | 2.2 | 375.1 | M + H | std method |
| | benzofuran-2(3H)-ylidene)methyl]- | | | | w/formic |
| | 2-methyl-1H-indol-1-yl}butanenitrile | | | | |
| 241 | 4-{3-[(4,6-dihydroxy-3-oxo-1- | 2.31 | 361.1 | M + H | std method |
| | benzofuran-2(3H)-ylidene)methyl]- | | | | w/formic |
| | 1H-indol-1-yl}butanenitrile | | | | |
| 242 | 2-{[7-(benzyloxy)-1H-indol-3- | 2.41 | 400.1 | M + H | std method |
| | yl]methylene}-4,6-dihydroxy-1- | | | | w/formic |
| | benzofuran-3(2H)-one | | | | |
| 243 | 2-{[4-(benzyloxy)-1H-indol-3- | 2.38 | 400.1 | M + H | std method |
| | yl]methylene}-4,6-dihydroxy-1- | | | | w/formic |
| | benzofuran-3(2H)-one | | | | |
| 244 | 2-[(7-bromo-1H-indol-3- | 2.25 | 372 | M + H | std method |
| | yl)methylene]-4,6-dihydroxy-1- | | | | w/formic |
| | benzofuran-3(2H)-one | | | | |
| 245 | methyl 3-[(4,6-dihydroxy-3-oxo-1- | 2.22 | 352.1 | M + H | std method |
| | benzofuran-2(3H)-ylidene)methyl]- | | | | w/formic |
| | 1H-indole-7-carboxylate | | | | |
| 246 | 4,6-dihydroxy-2-[(7-hydroxy-1H- | 1.9 | 310.1 | M + H | std method |
| | indol-3-yl)methylene]-1- | | | | w/formic |
| | benzofuran-3(2H)-one | | | | |
| 247 | 2-[(1,2-dimethyl-1H-indol-3- | 2.23 | 322.1 | M + H | std method |
| | yl)methylene]-4,6-dihydroxy-1- | | | | w/formic |
| | benzofuran-3(2H)-one | | | | |
| 248 | 2-[(5-bromo-1-methyl-1H-indol-3- | 2.37 | 386 | M + H | std method |
| | yl)methylene]-4,6-dihydroxy-1- | | | | w/formic |
| | benzofuran-3(2H)-one | | | | |
| 249 | 2-[(7-bromo-1-methyl-1H-indol-3- | 2.44 | 386 | M + H | std method |
| | yl)methylene]-4,6-dihydroxy-1- | | | | w/formic |
| | benzofuran-3(2H)-one | | | | |
| 250 | methyl 3-[(4,6-dihydroxy-3-oxo-1- | 2.25 | 366.1 | M + H | std method |
| | benzofuran-2(3H)-ylidene)methyl]- | | | | w/formic |
| | 1-methyl-1H-indole-7-carboxylate | | | | |
| 251 | 4,6-dihydroxy-2-[(7-methoxy-1- | 2.31 | 338.1 | M + H | std method |
| | methyl-1H-indol-3-yl)methylene]-1- | | | | w/formic |
| | benzofuran-3(2H)-one | | | | |
| 252 | 2-[(4-chloro-1-methyl-1H-indol-3- | 2.32 | 342 | M + H | std method |
| | yl)methylene]-4,6-dihydroxy-1- | | | | w/formic |
| | benzofuran-3(2H)-one | | | | |
| 253 | 2-[(4-bromo-1-methyl-1H-indol-3- | 2.34 | 386 | M + H | std method |
| | yl)methylene]-4,6-dihydroxy-1- | | | | w/formic |
| | benzofuran-3(2H)-one | | | | |
| 254 | 4,6-dihydroxy-2-[(4-hydroxy-1- | 2.07 | 324.1 | M + H | std method |
| | methyl-1H-indol-3-yl)methylene]-1- | | | | w/formic |
| | benzofuran-3(2H)-one | | | | |
| 255 | 4,6-dihydroxy-2-[(4-methoxy-1- | 2.25 | 338.1 | M + H | std method |
| | methyl-1H-indol-3-yl)methylene]-1- | | | | w/formic |
| | benzofuran-3(2H)-one | | | | |
| 256 | 2-{[4-(benzyloxy)-1-methyl-1H- | 2.51 | 414.1 | M + H | std method |
| | indol-3-yl]methylene}-4,6- | | | | w/formic |
| | dihydroxy-1-benzofuran-3(2H)-one | | | | |
| 257 | 4-{3-[(4,6-dihydroxy-3-oxo-1- | 2.03 | 377.1 | M + H | std method |
| | benzofuran-2(3H)-ylidene)methyl]- | | | | w/formic |
| | 4-hydroxy-1H-indol-1- | | | | |
| | yl}butanenitrile | | | | |
| 259 | 4,6-dihydroxy-2-[(2-{4-[2-(2- | 2.42 | 502.2 | M + H | std method |
| | methoxyethoxy)ethoxy]phenyl}-1- | | | | w/formic |
| | methyl-1H-indol-3-yl)methylene]-1- | | | | |
| | benzofuran-3(2H)-one | | | | |
| 260 | 2-({2-[4-(benzyloxy)phenyl]-1- | 2.64 | 490.2 | M + H | std method |
| | methyl-1H-indol-3-yl}methylene)- | | | | w/formic |
| | 4,6-dihydroxy-1-benzofuran-3(2H)- | | | | |
| | one | | | | |
| 261 | 4,6-dihydroxy-2-{[2-(4- | 2.57 | 442.2 | M + H | std method |
| | isopropoxyphenyl)-1-methyl-1H- | | | | w/formic |
| | indol-3-yl]methylene}-1- | | | | |
| | benzofuran-3(2H)-one | | | | |
| 262 | 3-[(4,6-dihydroxy-3-oxo-1- | 1.8 | 432.1 | M + H | std method |
| | benzofuran-2(3H)-ylidene)methyl]- | | | | w/formic |
| | 1-(2-morpholin-4-ylethyl)-1H- | | | | |
| | indole-4-carbonitrile | | | | |
| 263 | 3-[(4,6-dihydroxy-3-oxo-1- | | | | |
| | benzofuran-2(3H)-ylidene)methyl]- | | | | |
| | 1-(pyridin-4-ylmethyl)-1H-indole-4- | | | | |
| | carbonitrile | | | | |
| 264 | 1-(3-cyanopropyl)-3-[(4,6- | 2.18 | 386.1 | M + H | std method |
| | dihydroxy-3-oxo-1-benzofuran- | | | | w/formic |
| | 2(3H)-ylidene)methyl]-1H-indole-4- | | | | |
| | carbonitrile | | | | |
| 265 | 3-[(4,6-dihydroxy-3-oxo-1- | 1.75 | 390.1 | M + H | std method |
| | benzofuran-2(3H)-ylidene)methyl]- | | | | w/formic |
| | 1-[2-(dimethylamino)ethyl]-1H- | | | | |
| | indole-4-carbonitrile | | | | |
| 266 | 3-[(4,6-dihydroxy-3-oxo-1- | 2.28 | 377.1 | M + H | std method |
| | benzofuran-2(3H)-ylidene)methyl]- | | | | w/formic |
| | 1-(2-methoxyethyl)-1H-indole-4- | | | | |
| | carbonitrile | | | | |
| 267 | methyl 3-[(4,6-dihydroxy-3-oxo-1- | 2.34 | 366.1 | M + H | std method |
| | benzofuran-2(3H)-ylidene)methyl]- | | | | w/formic |
| | 1-methyl-1H-indole-4-carboxylate | | | | |
| 268 | 4,6-dihydroxy-2-[(1-methyl-4- | 2.4 | 384.1 | M + H | std method |
| | phenyl-1H-indol-3-yl)methylene]-1- | | | | w/formic |
| | benzofuran-3(2H)-one | | | | |
| 269 | 4,6-dihydroxy-2-[(4-phenyl-1H- | 2.25 | 370.1 | M + H | std method |
| | indol-3-yl)methylene]-1- | | | | w/formic |
| | benzofuran-3(2H)-one | | | | |

### 4,6-dihydroxy-2-[(5-methoxy-1H-indol-3-yl)methyl]-1-benzofuran-3(2H)-one (Example 117)

4,6-dihydroxy-2-((5-methoxy-1H-indol-3-yl)methylene)benzofuran-3(2H)-one (0.09 mmol) synthesized as in Example 1 in 10 mL MeOH and 2 mL dioxane was hydrogenated under 48 psi H₂ atmosphere for 24 hrs. The reaction was filtered and concentrated in a Speed-Vac. The resulting residue purified via preparative HPLC conditions to afford the title compound. LCMS RT=1.75 MS=324.1.

Using the procedure of Example 117, Examples 118 and 119 were also prepared. Compound and analytical data are show in Table II below.

TABLE II
Compounds Prepared According the Procedure of Example 117.

| | | | | | LCMS |
|---|---|---|---|---|---|
| Example | Name | Time (min) | Mass | Ion | Conditions |
| 117 | 4,6-dihydroxy-2-[(5-methoxy-1H- | 1.75 | 324.1 | M − H | std method |
| | indol-3-yl)methyl]-1-benzofuran- | | | | w/formic |
| | 3(2H)-one | | | | |
| 118 | 4,6-dihydroxy-2-[(5-methoxy-2- | 2.05 | 400.1 | M − H | std method |
| | phenyl-1H-indol-3-yl)methyl]-1- | | | | w/formic |
| | benzofuran-3(2H)-one | | | | |
| 119 | 4,6-dihydroxy-2-[(5-methoxy-2- | 1.8 | 338.1 | M − H | std method |
| | methyl-1H-indol-3-yl)methyl]-1- | | | | w/NH₄OAc |
| | benzofuran-3(2H)-one | | | | @280 nm |

### 2-[(2-methyl-1H-indol-3-yl)methylene]-1-benzothiophen-3(2H)-one (Example 272)

To the benzo[b]thiophen-3(2H)-one (0.4 mmol, 1 eq) and 2-methyl-1H-indole-3-carbaldehyde (0.4 mmol, 1 eq) in benzene (2 mL) was added a catalytic amount of piperidine (3 drops). The resulting mixture was stirred for 120 minutes at 90° C. and allowed to cool to room temperature. The solution was concentrated in a Speed-Vac and the resulting residue purified via preparative HPLC conditions to afford the title compound. LCMS RT=2.55 MS=290.

Using the procedure of Example 272, Examples 271 and 273-299 were also prepared. Compound and analytical data are show in Table III below.

TABLE III
Compounds Prepared According the Procedure of Example 272.

| | | Time | | | LCMS |
|---|---|---|---|---|---|
| Example | Name | (min) | Mass | Ion | Conditions |
| 271 | (2Z)-2-(1H-indol-3-ylmethylene)-1- | | | | |
| | benzothiophen-3(2H)-one | | | | |
| 272 | (2Z)-2-[(2-methyl-1H-indol-3- | 2.55 | 290.1 | M − H | std method |
| | yl)methylene]-1-benzothiophen- | | | | w/NH₄OAC |
| | 3(2H)-one | | | | |
| 273 | (2Z)-2-(1H-indol-3-ylmethylene)-1- | 2.46 | 276.1 | M − H | std method |
| | benzothiophen-3(2H)-one | | | | w/NH₄OAC |
| 274 | (2Z)-2-[(2-phenyl-1H-indol-3- | 2.72 | 352.1 | M − H | std method |
| | yl)methylene]-1-benzothiophen- | | | | w/NH₄OAC |
| | 3(2H)-one | | | | |
| 275 | (2Z)-2-[(1-methyl-2-phenyl-1H- | 2.86 | 368.1 | M + H | std method |
| | indol-3-yl)methylene]-1- | | | | w/NH₄OAC |
| | benzothiophen-3(2H)-one | | | | |
| 276 | (2Z)-2-{[2-(2-naphthyl)-1H-indol-3- | 2.85 | 402.1 | M − H | std method |
| | yl]methylene}-1-benzothiophen- | | | | w/NH₄OAC |
| | 3(2H)-one | | | | |
| 277 | (2Z)-2-{[2-(4-fluorophenyl)-1H- | 2.72 | 370.1 | M − H | std method |
| | indol-3-yl]methylene}-1- | | | | w/NH₄OAC |
| | benzothiophen-3(2H)-one | | | | |
| 278 | (2Z)-2-[(1-methyl-1H-indol-3- | 2.64 | 292.1 | M + H | std method |
| | yl)methylene]-1-benzothiophen- | | | | w/NH₄OAC @ |
| | 3(2H)-one | | | | 230 nm |
| 279 | (2Z)-2-[(6-methyl-1H-indol-3- | 2.56 | 290.1 | M − H | std method |
| | yl)methylene]-1-benzothiophen- | | | | w/NH₄OAC @ |
| | 3(2H)-one | | | | 230 nm |
| 280 | (2Z)-2-[(7-methyl-1H-indol-3- | 2.57 | 290.1 | M − H | std method |
| | yl)methylene]-1-benzothiophen- | | | | w/NH₄OAC |
| | 3(2H)-one | | | | |
| 281 | (2Z)-5-chloro-2-(1H-indol-3- | 2.67 | 310 | M − H | std method w/ |
| | ylmethylene)-1-benzothiophen- | | | | formic |
| | 3(2H)-one | | | | |
| 282 | (2Z)-2-[(1-benzyl-1H-indol-3- | 2.82 | 368.1 | M + H | std method |
| | yl)methylene]-1-benzothiophen- | | | | w/NH₄OAC |
| | 3(2H)-one | | | | |
| 283 | (2Z)-5-chloro-2-[(2-phenyl-1H- | 2.89 | 386 | M − H | std method |
| | indol-3-yl)methylene]-1- | | | | w/NH₄OAC |
| | benzothiophen-3(2H)-one | | | | |
| 284 | (2Z)-5-chloro-2-{[2-(4- | 2.92 | 420 | M − H | std method w/ |
| | chlorophenyl)-1H-indol-3- | | | | formic @230 nm |
| | yl]methylene}-1-benzothiophen- | | | | |
| | 3(2H)-one | | | | |
| 285 | (2Z)-5-chloro-2-{[2-(2-naphthyl)- | 3.02 | 436.1 | M − H | std method |
| | 1H-indol-3-yl]methylene}-1- | | | | w/NH₄OAC |
| | benzothiophen-3(2H)-one | | | | |
| 286 | (2Z)-5-chloro-2-{[2-(4- | 2.84 | 404 | M − H | std method w/ |
| | fluorophenyl)-1H-indol-3- | | | | formic |
| | yl]methylene}-1-benzothiophen- | | | | |
| | 3(2H)-one | | | | |
| 287 | (2Z)-2-[(1-benzyl-1H-indol-3- | 2.92 | 402.1 | M + H | std method w/ |
| | yl)methylene]-5-chloro-1- | | | | formic |
| | benzothiophen-3(2H)-one | | | | |
| 288 | (2Z)-2-{[5-(benzyloxy)-1H-indol-3- | 2.87 | 416.1 | M − H | std method w/ |
| | yl]methylene}-5-chloro-1- | | | | formic |
| | benzothiophen-3(2H)-one | | | | |
| 289 | (2Z)-2-(1H-indol-3-ylmethylene)-5- | 2.77 | 292.1 | M + H | std method w/ |
| | methyl-1-benzothiophen-3(2H)-one | | | | formic |
| 290 | (2Z)-5-methyl-2-[(2-phenyl-1H- | 2.78 | 368.1 | M + H | std method w/ |
| | indol-3-yl)methylene]-1- | | | | formic |
| | benzothiophen-3(2H)-one | | | | |
| 291 | (2Z)-2-{[2-(4-chlorophenyl)-1H- | 2.85 | 400.1 | M − H | std method w/ |
| | indol-3-yl]methylene}-5-methyl-1- | | | | formic |
| | benzothiophen-3(2H)-one | | | | |
| 292 | (2Z)-5-methyl-2-[(6-methyl-1H- | 2.65 | 306.1 | M + H | std method w/ |
| | indol-3-yl)methylene]-1- | | | | formic |
| | benzothiophen-3(2H)-one | | | | |
| 293 | (2Z)-5-methyl-2-[(7-methyl-1H- | 2.65 | 306.1 | M + H | std method w/ |
| | indol-3-yl)methylene]-1- | | | | formic |
| | benzothiophen-3(2H)-one | | | | |
| 294 | (2Z)-2-[(5-bromo-1H-indol-3- | 2.75 | 368 | M − H | std method w/ |
| | yl)methylene]-5-methyl-1- | | | | formic |
| | benzothiophen-3(2H)-one | | | | |
| 295 | (2Z)-2-{[5-(benzyloxy)-1H-indol-3- | 2.76 | 398.1 | M + H | std method w/ |
| | yl]methylene}-5-methyl-1- | | | | formic |
| | benzothiophen-3(2H)-one | | | | |
| 296 | 5-methyl-2-{[2-(2-naphthyl)-1H- | 2.9 | 418.1 | M + H | std method w/ |
| | indol-3-yl]methylene}-1- | | | | formic |
| | benzothiophen-3(2H)-one | | | | |
| 297 | 2-{[2-(4-fluorophenyl)-1H-indol-3- | 2.82 | 384.1 | M − H | std method |
| | yl]methylene}-5-methyl-1- | | | | w/NH₄OAC |
| | benzothiophen-3(2H)-one | | | | |
| 298 | 5-methyl-2-[(2-methyl-5-nitro-1H- | 2.65 | 349.1 | M − H | std method w/ |
| | indol-3-yl)methylene]-1- | | | | formic |
| | benzothiophen-3(2H)-one | | | | |
| 299 | 5-methyl-2-[(1-methyl-1H-indol-3- | 2.76 | 306.1 | M + H | std method |
| | yl)methylene]-1-benzothiophen- | | | | w/NH₄OAC |
| | 3(2H)-one | | | | |

Preparative Reverse-Phase HPLC (RP-HPLC)

Compounds were in dissolved in 2 mL of 1:1 DMSO:MeCN, filtered through a 0.45 μm GMF, and purified on a Gilson HPLC, using a Phenomenex LUNA C₁₈ column: 60 mm×21.2 mm I.D., 5 um particle size: with ACN/H₂O (containing 0.2% TFA) gradient elution (95:5 H₂O:MeCN to 10:90 H₂O:MeCN; 8 minutes run

LCMS Conditions: standard method w/formic

HPLC Conditions: Instrument—Agilent 1100, Column: Thermo Aquasil C18, 50×2.1 mm, 5 um, Mobile Phase A: 0.1% Formic Acid in water, B: 0.1% Formic Acid in CAN, Flow Rate: 0.800 mL/min, Column Temperature: 40° C., Injection Volume: 5 mL, UV: monitor 215, 230, 254, 280, and 300 nm, Purity is reported at 254 nm unless otherwise noted.

Gradient Table:

| Time (min) | % B |
|---|---|
| 0 | 0 |
| 2.5 | 100 |
| 4.0 | 100 |
| 4.1 | 0 |
| 5.5 | 0 |

MS Conditions: Instrument: Agilent MSD; Ionization Mode: API-ES; Gas Temperature: 350° C.; Drying Gas: 11.0 L/min.; Nebulizer Pressure: 55 psig; Polarity: 50% positive, 50% negative; VCap: 3000V (positive), 2500V (negative); Fragmentor: 80 (positive), 120 (negative); Mass Range: 100-1000 m/z; Threshold: 150; Step size: 0.15; Gain: 1; Peak width: 0.15 min.

LCMS Conditions: standard method w/NH₄OAC

HPLC Conditions: Instrument—Agilent 1100, Column: Thermo Aquasil C18, 50×2.1 mm, 5 um, Mobile Phase A: 0.1% Ammonium Acetate in water, B: 0.1% Ammonium Acetate in CAN, Flow Rate: 0.800 mL/min, Column Temperature: 40° C., Injection Volume: 5 mL, UV: monitor 215, 230, 254, 280, and 300 nm. Purity is reported at 254 nm unless otherwise noted.

Gradient Table:

| Time (min) | % B |
|---|---|
| 0 | 0 |
| 2.5 | 100 |
| 4.0 | 100 |
| 4.1 | 0 |
| 5.5 | 0 |

MS Conditions: Instrument: Agilent MSD; Ionization Mode: API-ES; Gas Temperature: 350° C.; Drying Gas: 11.0 L/min.; Nebulizer Pressure: 55 psig; Polarity: 50% positive, 50% negative; VCap: 3000V (positive), 2500V (negative); Fragmentor: 80 (positive), 120 (negative); Mass Range: 100-1000 m/z; Threshold: 150; Step size: 0.15; Gain: 1; Peak width: 0.15 min.

### Condensation between 4,6-dihydroxy-benzofuran-3-one (Compound A) and 5-methoxy-indole-3-carbaldehydes

To a solution of the selected 5-methoxy-indole-3-carbaldehyde compounds (4 mmol, 1 eq.) and 4,6-dihydroxy-benzofuran-3-one A (664 mg, 4 mmol, 1 eq.) in EtOH (16 mL), a catalytic amount of 12 N HCl was added. The resulting mixture was stirred at 85° C. until disappearance of the starting materials and then allowed to cool to room temperature. The formed solid was recovered by filtration, washed with ethyl ether, and dried under vacuum. In some cases, further purification was necessary, as indicated in Table IV. According to this procedure, the following compounds were obtained:

TABLE IV

| | Reaction time | | | |
|---|---|---|---|---|
| Example # | (hours) | | Yield (%) | Purification |
| 302 | 12 | | 15 | Preparative |
| | | | | HPLC |
| 305 | 3 | | 31 | Filtration |
| 306 | 8 | | 42 | Trituration with |
| | | | | methylene chloride |
| | | | | and MeOH |
| 308 | 12 | | 10 | Preparative |
| | | | | HPLC |
| 309 | See Example | | See Example | See Example 108 |
| | 108 | | 108 | |
| 311 | 3 | | 52 | Trituration with |
| | | | | CHCl₃/Et₂OH 9:1 |
| 312 | 12 | | 35 | Filtration |
| 313 | 6 | | 74 | Filtration |
| 314 | 4 | | 20 | Filtration |
| 315 | 12 | | 71 | Filtration |
| 316 | 12 | | 42 | Preparative |
| | | | | HPLC |
| 317 | 12 | | 17 | Preparative |
| | | | | HPLC |
| 318 | 12 | | 48 | Filtration |
| 319 | 3 | | 75 | Filtration |
| 320 | 3 | | 56 | Filtration |
| 321 | 12 | | 20 | Preparative |
| | | | | HPLC |
| 322 | 2 | | 76 | Filtration |
| 323 | See Example | | See Example | See Example 114 |
| | 114 | | 114 | |
| 325 | 2 | | 8 | Trituration with |
| | | | | methylene chloride, |
| | | | | MeOH and hexane |
| 326 | 3 | | 30 | Filtration |
| 327 | 2 | | 75 | Filtration |
| 328 | 2 | | 82 | Filtration |
| 329 | 4 (rt) | | 49 | Double trituration |
| | | | | with methylene |
| | | | | chloride, MeOH and |
| | | | | hexane |
| 330 | 4 | | 34 | Double trituration |
| | | | | with methylene |
| | | | | chloride, MeOH and |
| | | | | hexane |
| 331 | 2 | | 34 | Trituration with |
| | | | | methylene chloride, |
| | | | | MeOH and hexane |
| 332 | 48 | | 60 | Filtration |
| 333 | 72 | | 63 | Filtration |
| 334 | 24 | | 37 | Filtration |
| 336 | 12 | | 26 | Preparative |
| | | | | HPLC |
| 337 | 48 | | 8 | Preparative |
| | | | | HPLC |
| 339 | 3 | | 25 | Trituration with |
| | | | | EtOAc |
| 340 | 6 | | 14 | Preparative |
| | | | | HPLC |
| 341 | 2 (rt) | | 41 | Filtration |
| 342 | 27 | | 65 | Filtration |
| 343 | 3 | | 38 | Trituration with |
| | | | | CHCl₃ |
| 344 | 2 | | 28 | Trituration with |
| | | | | EtOH, CHCl₃ and |
| | | | | hexane |
| 346 | 3 | | 58 | Trituration with |
| | | | | CH₃CN and MeOH |
| 348 | 30 | min | 7 | * |
| 353 | 2 | | 29 | Filtration |
| 354 | 4 | | 14 | Preparative HPLC |
| 360 | 12 | | 14 | Preparative HPLC |
| 410 | | | 26 | Preparative |
| | | | | HPLC |
| 430 | | | 55 | Trituration with Et₂O |
| | | | | and MeOH |
| 444 | 6 | | 38 | Filtration |
| 446 | | | 20 | Preparative |
| | | | | HPLC |
| 447 | | | 28 | Filtration |
| 463 | 2 | | 16 | Trituration with |
| | | | | EtOH, MeOH and |
| | | | | CH₃CN |

*After cooling the reaction mixture to room temperature, excess hexane was added and the mixture was stirred for 30 minutes. The formed solid was removed by filtration and the solvents were evaporated. The residue was purified by preparative HPLC.

### Example 300 (2Z)-2-({4-[4′-(Aminomethyl)biphenyl-4-yl]-1-methyl-1H-indol-3-yl}methylene)-4,6-dihydroxy-1-benzofuran-3(2H)-one

### Example 301 (2Z)-2-[(4-{4′-[(Dimethylamino)methyl]biphenyl-4-yl}-1-methyl-1H-indol-3-yl)methylene]-4,6-dihydroxy-1-benzofuran-3(2H)-one

### Example 308 (2Z)-4,6-Dihydroxy-2-({5-methoxy-1-[2-(4-methylpiperazin-1-yl)ethyl]-1H-indol-3-yl}methylene)-1-benzofuran-3(2H)-one

MS (m/z): 450.1 (MH⁺).

### Example 309 (2Z)-4,6-Dihydroxy-2-({5-methoxy-2-methyl-1-[2-(4-methylpiperazin-1-yl)ethyl]-1H-indol-3-yl}methylene)-1-benzofuran-3(2H)-one

MS (m/z): See Example 108

### Example 316 3-[(Z)-(4,6-Dihydroxy-3-oxo-1-benzofuran-2(3H)-yl idene)methyl]-5-methoxy-N,N-dimethyl-1-[2-(4-methylpiperazin-1-yl)ethyl]-1H-indole-2-carboxamide

MS (m/z): 521.2 (MH⁺).

### Example 302 (2Z)-2-({2-Cyclopropyl-5-methoxy-1-[2-(4-methylpiperazin-1-yl)ethyl]-1H-indol-3-yl}methylene)-4,6-dihydroxy-1-benzofuran-3(2H)-one

MS (m/z): 490.4 (MH⁺).

### Example 336 (2Z)-4,6-Dihydroxy-2-({5-methoxy-1-[2-(4-methylpiperazin-1-yl)ethyl]-2-(trifluoromethyl)-1H-indol-3-yl}methylene)-1-benzofuran-3(2H)-one

MS (m/z): 518.2 (MH⁺).

### Example 341 (2Z)-4,6-Dihydroxy-2-({5-methoxy-1-[2-(4-methylpiperazin-1-yl)ethyl]-2-(1-methyl-1H-pyrazol-4-yl)-1H-indol-3-yl}methylene)-1-benzofuran-3(2H)-one

MS (m/z): 530.1 (MH⁺).

### Example 344 (2Z)-2-({2-(3,5-Dimethylisoxazol-4-yl)-5-methoxy-1-[2-(4-methylpiperazin-1-yl)ethyl]-1H-indol-3-yl}methylene)-4,6-dihydroxy-1-benzofuran-3(2H)-one

MS (m/z): 545.1 (MH⁺).

### Example 463 (2Z)-4,6-Dihydroxy-2-({5-methoxy-1-[2-(4-methylpiperazin-1-yl)ethyl]-2-pyrimidin-5-yl-1H-indol-3-yl}methylene)-1-benzofuran-3(2H)-one

MS (m/z): 528.3 (MH⁺).

### Example 312 (2Z)-4,6-Dihydroxy-2-({1-[2-(1H-imidazol-1-yl)ethyl]-5-methoxy-1H-indol-3-yl}methylene)-1-benzofuran-3(2H)-one

MS (m/z): 418.3 (MH⁺).

### Example 328 (2Z)-4,6-Dihydroxy-2-({1-[2-(1H-imidazol-1-yl)ethyl]-5-methoxy-2-methyl-1H-indol-3-yl}methylene)-1-benzofuran-3(2H)-one

MS (m/z): 432.1 (MH⁺).

### Example 314 3-[(Z)-(4,6-Dihydroxy-3-oxo-1-benzofuran-2(3H)-ylidene)methyl]-1-[2-(1H-imidazol-1-yl)ethyl]-5-methoxy-N,N-dimethyl-1H-indole-2-carboxamide

MS (m/z): 489.3 (MH⁺).

### Example 334 (2Z)-2-({2-Cyclopropyl-1-[2-(1H-imidazol-1-yl)ethyl]-5-methoxy-1H-indol-3-yl}methylene)-4,6-dihydroxy-1-benzofuran-3(2H)-one

MS (m/z): 458.1 (MH⁺).

### Example 326 (2Z)-4,6-Dihydroxy-2-({1-[2-(3-hydroxypyrrolidin-1-yl)ethyl]-5-methoxy-1H-indol-3-yl}methylene)-1-benzofuran-3(2H)-one

MS (m/z): 437.3 (MH⁺).

### Example 327 (2Z)-4,6-Dihydroxy-2-({1-[2-(3-hydroxypyrrolidin-1-yl)ethyl]-5-methoxy-2-methyl-1H-indol-3-yl}methylene)-1-benzofuran-3(2H)-one

MS (m/z): 451.2 (MH⁺).

### Example 306 3-[(Z)-(4,6-Dihydroxy-3-oxo-1-benzofuran-2(3H)-yl idene)methyl]-1-[2-(3-hydroxypyrrolidin-1-yl)ethyl]-5-methoxy-N,N-dimethyl-1H-indole-2-carboxamide

MS (m/z): 508.2 (MH⁺).

### Example 342 (2Z)-2-({2-Cyclopropyl-1-[2-(3-hydroxypyrrolidin-1-yl)ethyl]-5-methoxy-1H-indol-3-yl}methylene)-4,6-dihydroxy-1-benzofuran-3(2H)-one

MS (m/z): 477.2 (MH⁺).

### Example 340 (2Z)-2-({2-Cyclopropyl-5-methoxy-1-[2-(1H-pyrazol-1-yl)ethyl]-1H-indol-3-yl}methylene)-4,6-dihydroxy-1-benzofuran-3(2H)-one

MS (m/z): 458.1 (MH⁺).

### Example 337 (2Z)-4,6-Dihydroxy-2-({5-methoxy-1-[2-(1H-pyrazol-1-yl)ethyl]-2-(trifluoromethyl)-1H-indol-3-yl}methylene)-1-benzofuran-3(2H)-one

MS (m/z): 486.0 (MH⁺).

### Example 318 2-{3-[(Z)-(4,6-Dihydroxy-3-oxo-1-benzofuran-2(3H)-yl idene)methyl]-5-methoxy-1H-indol-1-yl}-N,N-dimethylacetamide

MS (m/z): 409.4 (MH⁺).

### Example 322 2-{3-[(Z)-(4,6-Dihydroxy-3-oxo-1-benzofuran-2(3H)-yl idene)methyl]-5-methoxy-2-methyl-1H-indol-1-yl}-N,N-dimethylacetamide

MS (m/z): 423.2 (MH⁺).

### Example 325 3-[(Z)-(4,6-Dihydroxy-3-oxo-1-benzofuran-2(3H)-yl idene)methyl]-1-[2-(dimethylamino)-2-oxoethyl]-5-methoxy-N,N-dimethyl-1H-indole-2-carboxamide

MS (m/z): 480.1 (MH⁺).

### Example 444 2-{2-Cyclopropyl-3-[(Z)-(4,6-dihydroxy-3-oxo-1-benzofuran-2(3H)-yl idene)methyl]-5-methoxy-1H-indol-1-yl}-N,N-dimethylacetamide

MS (m/z): 449.2 (MH⁺).

### Example 333 2-{3-[(Z)-(4,6-Dihydroxy-3-oxo-1-benzofuran-2(3H)-yl idene)methyl]-5-methoxy-2-(trifluoromethyl)-1H-indol-1-yl}-N,N-dimethylacetamide

MS (m/z): 477.0 (MH⁺).

### Example 329 2-{3-[(Z)-(4,6-Dihydroxy-3-oxo-1-benzofuran-2(3H)-yl idene)methyl]-5-methoxy-2-(1-methyl-1H-pyrazol-4-yl)-1H-indol-1-yl}-N,N-dimethylacetamide

MS (m/z): 489.2 (MH⁺).

### Example 319 (2Z)-4,6-Dihydroxy-2-({1-[2-(4-hydroxypiperidin-1-yl)ethyl]-5-methoxy-1H-indol-3-yl}methylene)-1-benzofuran-3(2H)-one

MS (m/z): 451.2 (MH⁺).

### Example 320 (2Z)-4,6-Dihydroxy-2-({1-[2-(4-hydroxypiperidin-1-yl)ethyl]-5-methoxy-2-methyl-1H-indol-3-yl}methylene)-1-benzofuran-3(2H)-one

MS (m/z): 465.3 (MH⁺).

### Example 311 3-[(Z)-(4,6-Dihydroxy-3-oxo-1-benzofuran-2(3H)-yl idene)methyl]-1-[2-(4-hydroxypiperidin-1-yl)ethyl]-5-methoxy-N,N-dimethyl-1H-indole-2-carboxamide

MS (m/z): 522.4 (MH⁺).

### Example 315 (2Z)-2-({2-Cyclopropyl-1-[2-(4-hydroxypiperidin-1-yl)ethyl]-5-methoxy-1H-indol-3-yl}methylene)-4,6-dihydroxy-1-benzofuran-3(2H)-one

MS (m/z): 491.5 (MH⁺).

### Example 317 (2Z)-4,6-Dihydroxy-2-{[1-(2-hydroxyethyl)-5-methoxy-1H-indol-3-yl]methylene}-1-benzofuran-3(2H)-one

MS (m/z): 368.1 (MH⁺).

### Example 321 (2Z)-4,6-Dihydroxy-2-{[1-(2-hydroxyethyl)-5-methoxy-2-methyl-1H-indol-3-yl]methylene}-1-benzofuran-3(2H)-one

MS (m/z): 382.2 (MH⁺).

### Example 348 3-[(Z)-(4,6-Dihydroxy-3-oxo-1-benzofuran-2(3H)-yl idene)methyl]-1-(2-hydroxyethyl)-5-methoxy-N,N-dimethyl-1H-indole-2-carboxamide

MS (m/z): 439.4 (MH⁺).

### Example 313 (2Z)-2-{[2-Cyclopropyl-1-(2-hydroxyethyl)-5-methoxy-1H-indol-3-yl]methylene}-4,6-dihydroxy-1-benzofuran-3(2H)-one

MS (m/z): 408.4 (MH⁺).

### Example 332 (2Z)-4,6-Dihydroxy-2-{[1-(2-hydroxyethyl)-5-methoxy-2-(trifluoromethyl)-1H-indol-3-yl]methylene}-1-benzofuran-3(2H)-one

MS (m/z): 436.0 (MH⁺).

### Example 323 (2Z)-2-({1-[3-(Dimethylamino)propyl]-5-methoxy-1H-indol-3-yl}methylene)-4,6-dihydroxy-1-benzofuran-3(2H)-one

MS (m/z): See Example 114

### Example 305 (2Z)-2-({1-[3-(Dimethylamino)propyl]-5-methoxy-2-methyl-1H-indol-3-yl}methylene)-4,6-dihydroxy-1-benzofuran-3(2H)-one

MS (m/z): 423.3 (MH⁺).

### Example 343 3-[(Z)-(4,6-Dihydroxy-3-oxo-1-benzofuran-2(3H)-yl idene)methyl]-1-[3-(dimethylamino)propyl]-5-methoxy-N,N-dimethyl-1H-indole-2-carboxamide

MS (m/z): 480.1 (MH⁺).

### Example 330 (2Z)-4,6-Dihydroxy-2-{[5-methoxy-2-(1-methyl-1H-pyrazol-4-yl)-1H-indol-3-yl]methylene}-1-benzofuran-3(2H)-one

MS (m/z): 404.1 (MH⁺).

### Example 360 (2Z)-2-{[2-(3,5-Dimethyl isoxazol-4-yl)-5-methoxy-1H-indol-3-yl]methylene}-4,6-dihydroxy-1-benzofuran-3(2H)-one

MS (m/z): 419.1 (MH⁺).

### Example 354 (2Z)-4,6-Dihydroxy-2-[(5-methoxy-2-pyrimidin-5-yl-1H-indol-3-yl)methylene]-1-benzofuran-3(2H)-one

MS (m/z): 402.1 (MH⁺).

### Example 331 (2Z)-4,6-Dihydroxy-2-({5-methoxy-2-[(4-methylpiperazin-1-yl)carbonyl]-1H-indol-3-yl}methylene)-1-benzofuran-3(2H)-one

MS (m/z): 450.1 (MH⁺).

### Example 346 (2Z)-4,6-Dihydroxy-2-({5-methoxy-2-[(4-methylpiperazin-1-yl)methyl]-1H-indol-3-yl}methylene)-1-benzofuran-3(2H)-one

MS (m/z): 436.1 (MH⁺).

### Example 353 (2Z)-2-({2-[(Dimethylamino)methyl]-5-methoxy-1H-indol-3-yl}methylene)-4,6-dihydroxy-1-benzofuran-3(2H)-one

MS (m/z): 381.1 (MH⁺).

### Example 339 3-[(Z)-(4,6-Dihydroxy-3-oxo-1-benzofuran-2(3H)-yl idene)methyl]-5-methoxy-1H-indole-2-carboxylic acid

MS (m/z): 368.1 (MH⁺).

### Example 384 (2Z)-6-Methoxy-2-[(5-methoxy-1H-indol-3-yl)methylene]-1-benzofuran-3(2H)-one

MS (m/z): 322.2 (MH⁺)

### Example 410 (2Z)-2-({2-cyclopentyl-5-methoxy-1-[2-(4-methylpiperazin-1-yl)ethyl]-1H-indol-3-yl}methylene)-4,6-dihydroxy-1-benzofuran-3(2H)-one

MS (m/z): 518.3 (MH⁺).

### Example 414 (2Z)-6-Hydroxy-2-[(5-methoxy-1H-indol-3-yl)methylene]-1-benzofuran-3(2H)-one

MS (m/z): 308.2 (MH⁺)

### Example 426 N-{4-[(2Z)-2-({1-[3-(Dimethylamino)propyl]-5-methoxy-1H-indol-3-yl}methylene)-3-oxo-2,3-dihydro-1-benzofuran-6-yl]phenyl}acetamide

MS (m/z): 510.4 (MH⁺)

### Example 427 (2Z)-6-(2-Aminopyrimidin-5-yl)-2-({1-[3-(dimethylamino)propyl]-5-methoxy-1H-indol-3-yl}methylene)-1-benzofuran-3(2H)-one

MS (m/z): 470.4 (MH⁺)

### Example 430 (2Z)-4,6-Dihydroxy-2-({5-methoxy-1-[2-(4-methylpiperazin-1-yl)ethyl]-2-(pyrrolidin-1-ylcarbonyl)-1H-indol-3-yl}methylene)-1-benzofuran-3(2H)-one

MS (m/z): 547.2 (MH⁺).

### Example 433 6-Methoxy-2-[(5-methoxy-1H-indol-3-yl)methylene]-1-benzothiophen-3(2H)-one

MS (m/z): 338.2 (MH⁺)

### Example 434 2-[(5-Methoxy-1H-indol-3-yl)methylene]-6-(methylthio)-1-benzofuran-3(2H)-one

MS (m/z): 338.2 (MH⁺)

### Example 436 2-[(5-Methoxy-1H-indol-3-yl)methylene]-6-(methylsulfonyl)-1-benzofuran-3(2H)-one

MS (m/z): 370.2 (MH⁺)

### Example 446 (2Z)-2-({2-Cyclobutyl-5-methoxy-1-[2-(4-methylpiperazin-1-yl)ethyl]-1H-indol-3-yl}methylene)-4,6-dihydroxy-1-benzofuran-3(2H)-one

MS (m/z): 504.3 (MH⁺).

### Example 447 (2Z)-2-({2-Cyclohexyl-5-methoxy-1-[2-(4-methylpiperazin-1-yl)ethyl]-1H-indol-3-yl}methylene)-4,6-dihydroxy-1-benzofuran-3(2H)-one

MS (m/z): 532.3 (MH⁺).

### Condensation between mono-hydroxy-benzofuran-3-ones and 5-methoxy-indole-3-carbaldehydes 6-mono-hydroxy derivatives

Following the previously described conditions for the condensation, the following 6-mono-hydroxy derivatives were obtained (commercially available 6-hydroxy-benzofuran-3-one was used).

TABLE V

| | Reaction | | | |
|---|---|---|---|---|
| | time | | | |
| Example # | (hours) | | Yield % | Purification |
| 303 | 12 | | 59 | Filtration |
| 307 | 3 | | 75 | Filtration |
| 310 | 1 | | 78 | Filtration |
| 324 | 24 | | 76 | Filtration |
| 335 | 36 | | 65 | Filtration |
| 345 | 5 | | 56 | Filtration |
| 355 | 6 | | 19 | Trituration with |
| | | | | CH₃CN |
| 356 | 15 | min | 31 | Filtration |
| 357 | 15 | min | 35 | Filtration |
| 358 | 15 | min | 46 | Filtration |
| 362 | 6 | | 76 | Filtration |
| 411 | | | 60 | Filtration |
| 412 | | | 42 | Trituration with |
| | | | | methylene chloride |
| 452 | 1 | | 60 | Filtration |
| 453 | 1 | | 50 | Filtration |
| 454 | 1 | | 32 | Trituration with |
| | | | | MeOH, methylene |
| | | | | chloride and hexane |
| 455 | 1 | | 58 | Filtration |
| 460 | 15 | min | 29 | Filtration |
| 461 | 15 | min | 50 | Filtration |
| 462 | 8 | | 40 | Filtration |
| 469 | 45 | min | 62 | Filtration |
| 470 | 1 | | 46 | Filtration |

### Example 310 (2Z)-6-Hydroxy-2-[(5-methoxy-2-phenyl-1H-indol-3-yl)methylene]-1-benzofuran-3(2H)-one

MS (m/z): 383.4 (MH⁺).

### Example 303 (2Z)-6-Hydroxy-2-({5-methoxy-2-methyl-1-[2-(4-methylpiperazin-1-yl)ethyl]-1H-indol-3-yl}methylene)-1-benzofuran-3(2H)-one

MS (m/z): 448.2 (MH⁺).

### Example 335 3-[(Z)-(6-Hydroxy-3-oxo-1-benzofuran-2(3H)-yl idene)methyl]-5-methoxy-N,N-dimethyl-1-[2-(4-methylpiperazin-1-yl)ethyl]-1H-indole-2-carboxamide

MS (m/z): 505.2 (MH⁺).

### Example 362 (2Z)-2-({2-Cyclopropyl-5-methoxy-1-[2-(4-methylpiperazin-1-yl)ethyl]-1H-indol-3-yl}methylene)-6-hydroxy-1-benzofuran-3(2H)-one

MS (m/z): 474.2 (MH⁺).

### Example 469 (2Z)-2-({2-(3,5-Dimethylisoxazol-4-yl)-5-methoxy-1-[2-(4-methylpiperazin-1-yl)ethyl]-1H-indol-3-yl}methylene)-6-hydroxy-1-benzofuran-3(2H)-one

MS (m/z): 529.2 (MH⁺).

### Example 355 (2Z)-2-({2-Cyclopropyl-5-methoxy-1-[2-(1H-pyrazol-1-yl)ethyl]-1H-indol-3-yl}methylene)-6-hydroxy-1-benzofuran-3(2H)-one

MS (m/z): 442.2 (MH⁺).

### Example 452 (2Z)-6-Hydroxy-2-{[5-methoxy-2-methyl-1-(2-pyrrolidin-1-ylethyl)-1H-indol-3-yl]methylene}-1-benzofuran-3(2H)-one

MS (m/z): 419.2 (MH⁺).

### Example 453 (2Z)-6-Hydroxy-2-({5-methoxy-2-methyl-1-[2-(2-methylpyrrolidin-1-yl)ethyl]-1H-indol-3-yl}methylene)-1-benzofuran-3(2H)-one

MS (m/z): 433.3 (MH⁺).

### Example 454 (2Z)-6-Hydroxy-2-{[5-methoxy-2-methyl-1-(2-piperidin-1-ylethyl)-1H-indol-3-yl]methylene}-1-benzofuran-3(2H)-one

MS (m/z): 433.3 (MH⁺).

### Example 455 (2Z)-6-Hydroxy-2-({5-methoxy-2-methyl-1-[2-(4-methylpiperidin-1-yl)ethyl]-1H-indol-3-yl}methylene)-1-benzofuran-3(2H)-one

MS (m/z): 447.3 (MH⁺).

### Example 470 (2Z)-2-{[1-(2-Azepan-1-ylethyl)-5-methoxy-2-methyl-1H-indol-3-yl]methylene}-6-hydroxy-1-benzofuran-3(2H)-one

MS (m/z): 447.2 (MH⁺).

### Example 324 (2Z)-2-({1-[3-(Dimethylamino)propyl]-5-methoxy-1H-indol-3-yl}methylene)-6-hydroxy-1-benzofuran-3(2H)-one

MS (m/z): 393.2 (MH⁺).

### Example 307 (2Z)-2-({1-[3-(Dimethylamino)propyl]-5-methoxy-2-methyl-1H-indol-3-yl}methylene)-6-hydroxy-1-benzofuran-3(2H)-one

MS (m/z): 407.0 (MH⁺).

### Example 345 1-[3-(Dimethylamino)propyl]-3-[(Z)-(6-hydroxy-3-oxo-1-benzofuran-2(3H)-yl idene)methyl]-5-methoxy-N,N-dimethyl-1H-indole-2-carboxamide

MS (m/z): 464.1 (MH⁺).

### Example 462 (2Z)-2-({2-Cyclopropyl-1-[3-(dimethylamino)propyl]-5-methoxy-1H-indol-3-yl}methylene)-6-hydroxy-1-benzofuran-3(2H)-one

MS (m/z): 433.1 (MH⁺).

### Example 356 (2Z)-6-Hydroxy-2-{[5-methoxy-2-methyl-1-(3-pyrrolidin-1-yl propyl)-1H-indol-3-yl]methylene}-1-benzofuran-3(2H)-one

MS (m/z): 433.2 (MH⁺).

### Example 461 (2Z)-6-Hydroxy-2-({5-methoxy-2-methyl-1-[3-(2-methylpyrrolidin-1-yl)propyl]-1H-indol-3-yl}methylene)-1-benzofuran-3(2H)-one

MS (m/z): 447.4 (MH⁺).

### Example 357 (2Z)-6-Hydroxy-2-{[5-methoxy-2-methyl-1-(3-piperidin-1-yl propyl)-1H-indol-3-yl]methylene}-1-benzofuran-3(2H)-one

MS (m/z): 447.2 (MH⁺).

### Example 460 (2Z)-6-Hydroxy-2-({5-methoxy-2-methyl-1-[3-(4-methylpiperidin-1-yl)propyl]-1H-indol-3-yl}methylene)-1-benzofuran-3(2H)-one

MS (m/z): 461.2 (MH⁺).

### Example 358 (2Z)-2-{[1-(3-Azepan-1-yl propyl)-5-methoxy-2-methyl-1H-indol-3-yl]methylene}-6-hydroxy-1-benzofuran-3(2H)-one

MS (m/z): 461.2 (MH⁺).

### Example 411 (2Z)-2-({2-Cyclopentyl-5-methoxy-1-[2-(4-methylpiperazin-1-yl)ethyl]-1H-indol-3-yl}methylene)-6-hydroxy-1-benzofuran-3(2H)-one

MS (m/z): 502.3 (MH⁺).

### Example 412 (2Z)-6-Hydroxy-2-({5-methoxy-1-[2-(4-methylpiperazin-1-yl)ethyl]-2-(morpholin-4-ylcarbonyl)-1H-indol-3-yl}methylene)-1-benzofuran-3(2H)-one

MS (m/z): 547.3 (MH⁺).

Following the previously described conditions for the condensation, the following 4-mono-hydroxy derivatives were was obtained (4-hydroxy-benzofuran-3-one, Compound B, was used).

### Example 304 (2Z)-4-Hydroxy-2-[(5-methoxy-2-phenyl-1H-indol-3-yl)methylene]-1-benzofuran-3(2H)-one

Reaction time 12 hours, 9% yield, purified by Preparative HPLC, MS (m/z): 384.2 (MH⁺).

### Condensation between substituted 6-hydroxy-benzofuranones and 5-methoxy-indole-3-carbaldehydes

Following the usual conditions for the condensation, the monosubstituted 6-hydroxy derivatives shown in Table VI were obtained, using monosubstituted benzofuranone compounds C-O:

TABLE VI

| | Reaction time | | |
|---|---|---|---|
| Example # | (hours) | Yield (%) | Purification |
| 338 | 7 | 81 | Filtration |
| 347 | 5 | 69 | Filtration |
| 349 | 4 | 67 | Filtration |
| 350 | 4 | 62 | Filtration |
| 351 | 4 | 61 | Filtration |
| 352 | 4 | 77 | Filtration |
| 359 | 12 | 72 | Filtration |
| 361 | 5 | 68 | Filtration |
| 429 | | 39 | Preparative |
| | | | HPLC |
| 448 | 6 | 85 | Filtration |
| 449 | 4 | 70 | Filtration |
| 450 | 12 | 57 | Filtration |
| 451 | 12 | 47 | Filtration |
| 456 | 12 | 78 | Filtration |
| 457 | 12 | 72 | Filtration |
| 458 | 12 | 83 | Filtration |
| 459 | 12 | 67 | Filtration |
| 464 | See Example | See Example | See Example |
| | 338 | 338 | 338 |
| 465 | 6 | 52 | Trituration with |
| | | | EtOH and |
| | | | CH₃CN |
| 466 | 6 | 64 | Filtration |
| 467 | 6 | 75 | Filtration |
| 468 | 5 | 53 | Trituration with |
| | | | methylene |
| | | | chloride |

### Example 338 (2Z)-6-Hydroxy-2-({5-methoxy-2-methyl-1-[2-(4-methylpiperazin-1-yl)ethyl]-1H-indol-3-yl}methylene)-4-methyl-1-benzofuran-3(2H)-one

MS (m/z): 462.2 (MH⁺).

### Example 458 (2Z)-6-Hydroxy-2-({5-methoxy-2-methyl-1-[2-(4-methylpiperazin-1-yl)ethyl]-1H-indol-3-yl}methylene)-5-methyl-1-benzofuran-3(2H)-one

MS (m/z): 462.3 (MH⁺).

### Example 347 (2Z)-6-Hydroxy-2-({5-methoxy-2-methyl-1-[2-(4-methylpiperazin-1-yl)ethyl]-1H-indol-3-yl}methylene)-7-methyl-1-benzofuran-3(2H)-one

MS (m/z): 462.2 (MH⁺).

### Example 359 (2Z)-4-Fluoro-6-hydroxy-2-({5-methoxy-2-methyl-1-[2-(4-methylpiperazin-1-yl)ethyl]-1H-indol-3-yl}methylene)-1-benzofuran-3(2H)-one

MS (m/z): 466.1 (MH⁺).

### Example 351 (2Z)-5-Fluoro-6-hydroxy-2-({5-methoxy-2-methyl-1-[2-(4-methylpiperazin-1-yl)ethyl]-1H-indol-3-yl}methylene)-1-benzofuran-3(2H)-one

MS (m/z): 466.1 (MH⁺).

### Example 457 (2Z)-7-Fluoro-6-hydroxy-2-({5-methoxy-2-methyl-1-[2-(4-methylpiperazin-1-yl)ethyl]-1H-indol-3-yl}methylene)-1-benzofuran-3(2H)-one

MS (m/z): 466.1 (MH⁺).

### Example 467 (2Z)-4-Chloro-6-hydroxy-2-({5-methoxy-2-methyl-1-[2-(4-methylpiperazin-1-yl)ethyl]-1H-indol-3-yl}methylene)-1-benzofuran-3(2H)-one

MS (m/z): 482.2 (MH⁺).

### Example 456 (2Z)-5-Chloro-6-hydroxy-2-({5-methoxy-2-methyl-1-[2-(4-methylpiperazin-1-yl)ethyl]-1H-indol-3-yl}methylene)-1-benzofuran-3(2H)-one

MS (m/z): 482.2 (MH⁺).

### Example 451 (2Z)-7-Chloro-6-hydroxy-2-({5-methoxy-2-methyl-1-[2-(4-methylpiperazin-1-yl)ethyl]-1H-indol-3-yl}methylene)-1-benzofuran-3(2H)-one

MS (m/z): 482.2 (MH⁺).

### Example 349 (2Z)-5-Bromo-6-hydroxy-2-({5-methoxy-2-methyl-1-[2-(4-methylpiperazin-1-yl)ethyl]-1H-indol-3-yl}methylene)-1-benzofuran-3(2H)-one

MS (m/z): 526.1 (MH⁺).

### Example 429 (2Z)-4-Bromo-6-hydroxy-2-({5-methoxy-2-methyl-1-[2-(4-methylpiperazin-1-yl)ethyl]-1H-indol-3-yl}methylene)-1-benzofuran-3(2H)-one

MS (m/z): 526.0 (MH⁺).

### Example 468 (2Z)-2-({1-[3-(Dimethylamino)propyl]-5-methoxy-2-methyl-1H-indol-3-yl}methylene)-6-hydroxy-4-methyl-1-benzofuran-3(2H)-one

MS (m/z): 421.2 (MH⁺).

### Example 459 (2Z)-2-({1-[3-(Dimethylamino)propyl]-5-methoxy-2-methyl-1H-indol-3-yl}methylene)-6-hydroxy-5-methyl-1-benzofuran-3(2H)-one

MS (m/z): 421.2 (MH⁺).

### Example 450 (2Z)-2-({1-[3-(Dimethylamino)propyl]-5-methoxy-2-methyl-1H-indol-3-yl}methylene)-6-hydroxy-7-methyl-1-benzofuran-3(2H)-one

MS (m/z): 421.3 (MH⁺).

### Example 465 (2Z)-2-({1-[3-(Dimethylamino)propyl]-5-methoxy-2-methyl-1H-indol-3-yl}methylene)-4-fluoro-6-hydroxy-1-benzofuran-3(2H)-one

MS (m/z): 425.2 (MH⁺).

### Example 352 (2Z)-2-({1-[3-(Dimethylamino)propyl]-5-methoxy-2-methyl-1H-indol-3-yl}methylene)-5-fluoro-6-hydroxy-1-benzofuran-3(2H)-one

MS (m/z): 425.2 (MH⁺).

### Example 449 (2Z)-2-({1-[3-(Dimethylamino)propyl]-5-methoxy-2-methyl-1H-indol-3-yl}methylene)-7-fluoro-6-hydroxy-1-benzofuran-3(2H)-one

MS (m/z): 425.2 (MH⁺).

### Example 466 (2Z)-4-Chloro-2-({1-[3-(dimethylamino)propyl]-5-methoxy-2-methyl-1H-indol-3-yl}methylene)-6-hydroxy-1-benzofuran-3(2H)-one

MS (m/z): 441.2 (MH⁺).

### Example 448 (2Z)-5-Chloro-2-({1-[3-(dimethylamino)propyl]-5-methoxy-2-methyl-1H-indol-3-yl}methylene)-6-hydroxy-1-benzofuran-3(2H)-one

MS (m/z): 441.2 (MH⁺).

### Example 361 (2Z)-7-Chloro-2-({1-[3-(dimethylamino)propyl]-5-methoxy-2-methyl-1H-indol-3-yl}methylene)-6-hydroxy-1-benzofuran-3(2H)-one

MS (m/z): 441.2 (MH⁺).

### Example 350 (2Z)-5-Bromo-2-({1-[3-(dimethylamino)propyl]-5-methoxy-2-methyl-1H-indol-3-yl}methylene)-6-hydroxy-1-benzofuran-3(2H)-one

MS (m/z): 485.1 (MH⁺).

### Example 464 (2Z)-2-({2-Cyclopropyl-5-methoxy-1-[2-(4-methylpiperazin-1-yl)ethyl]-1H-indol-3-yl}methylene)-6-hydroxy-4-methyl-1-benzofuran-3(2H)-one

MS (m/z): 488.3 (MH⁺).

### Example 364 The preparation of (2Z)-2-{[4-(4-Fluorophenyl)-1-methyl-1H-indol-3-yl]methylene}-4,6-dihydroxy-1-benzofuran-3(2H)-one

Step 1

### Preparation of 2-[(4-bromo-1-methyl-1H-indol-3-yl)methylene]-4,6-dihydroxy-1-benzofuran-3(2H)-one

A mixture of 2 g (12.04 mmol) of 4,6-dihydroxycoumaranone, 3.15 g (13.24 mmol) of 4-bromo-1-methyl-H-indole-3-carbaldehyde, 2.5 mL of conc. HCl, and 47.5 mL of absolute ethanol was stirred at 80° C. overnight. After cooling, the precipitate was filtered and washed with 10% methanol in methylene chloride. The solid was dried under house vacuum to give 3.8 g of yellow solid (82% yield). MS (m/z) 386.2 (MH⁺).

Step 2

A mixture of 120 mg (0.31 mmol) of 2-[(4-bromo-1-methyl-1H-indol-3-yl)methylene]-4,6-dihydroxy-1-benzofuran-3(2H)-one (WAC-575806), 86.5 mg (0.62 mmol) of 4-fluorophenyl boronic acid, 53.7 mg (0.047 mmol) of tetrakis(triphenylphosphine)palladium(0), and saturated aqueous sodium carbonate (1 mL), was placed in a microwave vial. To the mixture were added 3 mL of 1-methyl-2-pyrrolidinone and 1,2-dimethoxyethane (1:3). The sealed tube was heated by microwave for twenty minutes at 120° C. After cooling, the mixture was filtered through Celite™ and washed with 12% methanol in methylene chloride. After the solvent was evaporated, the residue was purified by column chromatography (10% methanol in ethyl acetate) to give 55 mg of a yellow solid (44% yield). MS (m/z) 402.2 (MH⁺).

The Following Final Compounds were Synthesized Using the Procedure for Example 364

### Example 363 (2Z)-2-{[4-(4-fluorophenyl)-1H-indol-3-yl]methylene}-4,6-dihydroxy-1-benzofuran-3(2H)-one

MS (m/z): 388.1 (MH⁺).

### Example 365 (2Z)-2-{[4-(3-Fluorophenyl)-1-methyl-1H-indol-3-yl]methylene}-4,6-dihydroxy-1-benzofuran-3(2H)-one

MS (m/z) 402.2 (MH⁺).

### Example 377 4-{3-[(Z)-(4,6-Dihydroxy-3-oxo-1-benzofuran-2(3H)-yl idene)methyl]-1-methyl-1H-indol-4-yl}benzamide

HRMS: calcd for C25H18N2O5⁺ H⁺, 427.12885; found (ESI-FTMS, [M⁺H]1⁺), 427.12893;

### Example 378 (2Z)-2-{[4-(3-Furyl)-1-methyl-1H-indol-3-yl]methylene}-4,6-dihydroxy-1-benzofuran-3(2H)-one

MS (m/z) 374.2 (MH⁺).

### Example 383 N-(4-{3-[(Z)-(4,6-Dihydroxy-3-oxo-1-benzofuran-2(3H)-yl idene)methyl]-1-methyl-1H-indol-4-yl}phenyl)acetamide

HRMS: calcd for C26H20N2O5+H⁺, 441.14450; found (ESI-FTMS, [M+H]⁺), 441.14452;

### Example 387 4-{3-[(Z)-(4,6-Dihydroxy-3-oxo-1-benzofuran-2(3H)-yl idene)methyl]-1-methyl-1H-indol-4-yl}-N-[3-(dimethylamino)propyl]benzamide

MS (m/z) 512.2 (MH⁺).

### Example 390 (2Z)-4,6-Dihydroxy-2-{[4-(3-hydroxyphenyl)-1-methyl-1H-indol-3-yl]methylene}-1-benzofuran-3(2H)-one

MS (m/z) 400.1 (MH⁺).

### Example 391 Methyl (4-{3-[(Z)-(4,6-Dihydroxy-3-oxo-1-benzofuran-2(3H)-yl idene)methyl]-1-methyl-1H-indol-4-yl}phenyl)carbamate

MS (m/z) 457.2 (MH⁺).

### Example 388 N-(3-{3-[(Z)-(4,6-Dihydroxy-3-oxo-1-benzofuran-2(3H)-yl idene)methyl]-1-methyl-1H-indol-4-yl}phenyl)acetamide

HRMS: calcd for C26H20N2O5+H⁺, 441.14450; found (ESI-FTMS, [M+H]⁺), 441.14472;

### Example 392 4-{3-[(Z)-(4,6-dihydroxy-3-oxo-1-benzofuran-2(3H)-yl idene)methyl]-1-methyl-1H-indol-4-yl}-N-methylbenzamide

HRMS: calcd for C₂₆H20N2O5+H⁺, 441.14450; found (ESI-FTMS, [M+H]⁺), 441.14533;

### Example 393 1-(4-{3-[(Z)-(4,6-Dihydroxy-3-oxo-1-benzofuran-2(3H)-yl idene)methyl]-1-methyl-1H-indol-4-yl}phenyl)-3-methylurea

HRMS: calcd for C26H21N3O5+H⁺, 456.15540; found (ESI, [M+H]+Obs′d), 456.1553;

### Example 394 3-(4-{3-[(Z)-(4,6-dihydroxy-3-oxo-1-benzofuran-2(3H)-yl idene)methyl]-1-methyl-1H-indol-4-yl}phenyl)-1,1-dimethylurea

HRMS: calcd for C27H23N3O5+H+, 470.17105; found (ESI, [M+H]+ Obs′d), 470.1708;

### Example 395 4-{3-[(Z)-(4,6-dihydroxy-3-oxo-1-benzofuran-2(3H)-yl idene)methyl]-1-methyl-1H-indol-4-yl}-N-isopropylbenzamide

HRMS: calcd for C28H24N2O5+H+, 469.17580; found (ESI-FTMS, [M+H]1+), 469.17648;

### Example 396 (2Z)-4,6-Dihydroxy-2-({1-methyl-4-[4-(pyrrolidin-1-ylcarbonyl)phenyl]-1H-indol-3-yl}methylene)-1-benzofuran-3(2H)-one

HRMS: calcd for C29H24N2O5+H+, 481.17580; found (ESI-FTMS, [M+H]1+), 481.17657;

### Example 399 4-{3-[(Z)-(4,6-dihydroxy-3-oxo-1-benzofuran-2(3H)-yl idene)methyl]-1-methyl-1H-indol-4-yl}-N-(2-furyl methyl)benzamide

HRMS: calcd for C30H22N2O6+H⁺, 507.15506; found (ESI, [M+H]⁺ Obs′d), 507.1548;

### Example 401 1-cyclopropyl-3-(4-{3-[(Z)-(4,6-dihydroxy-3-oxo-1-benzofuran-2(3H)-ylidene)methyl]-1-methyl-1H-indol-4-yl}phenyl)urea

MS (m/z) 482.3 (MH⁺).

### Example 404 N-(4-{3-[(Z)-(4,6-dihydroxy-3-oxo-1-benzofuran-2(3H)-yl idene)methyl]-1-methyl-1H-indol-4-yl}phenyl)morpholine-4-carboxamide

MS (m/z) 512.4 (MH⁺).

### Example 381 Preparation of (2Z)-4,6-dihydroxy-2-{[4-(4-isopropoxyphenyl)-1-methyl-1H-indol-3-yl]methylene}-1-benzofuran-3(2H)-one

A mixture of 100 mg (0.66 mmol) of 4,6-dihydroxycoumaranone) 158 mg (0.66 mmol) of 4-(4-isopropoxy-phenyl)-1-methyl-1H-indole-3-carboxylaldehyde, 0.25 mL of conc. HCl, and 4.75 mL of absolute ethanol was stirred at 80° C. overnight. After cooling, the reddish mixture was evaporated and purified by reverse phase HPLC to give 103.5 mg of (2Z)-4,6-dihydroxy-2-{[4-(4-isopropoxyphenyl)-1-methyl-1H-indol-3-yl]methylene}-1-benzofuran-3(2H)-one as a yellow solid (77% yield). MS (m/z) 442.2 (MH⁺).

The following final compounds were synthesized using the procedure for Example 381

### Example 363 (2Z)-2-{[4-(4-Fluorophenyl)-1H-indol-3-yl]methylene}-4,6-dihydroxy-1-benzofuran-3(2H)-one

MS (m/z) 386.2 (MH−).

### Example 366 (2Z)-2-{[4-(2-Fluorophenyl)-1-methyl-1H-indol-3-yl]methylene}-4,6-dihydroxy-1-benzofuran-3(2H)-one

MS (m/z) 402.2 (MH⁺).

### Example 367 3-{3-[(Z)-(4,6-Dihydroxy-3-oxo-1-benzofuran-2(3H)-yl idene)methyl]-1-methyl-1H-indol-4-yl}benzonitrile

MS (m/z) 407.1 (MH−).

### Example 368 4-{3-[(Z)-(4,6-Dihydroxy-3-oxo-1-benzofuran-2(3H)-yl idene)methyl]-1-methyl-1H-indol-4-yl}benzonitrile

MS (m/z) 409.3 (MH⁺).

### Example 369 (2Z)-4,6-Dihydroxy-2-{[4-(6-methoxypyridin-3-yl)-1-methyl-1H-indol-3-yl]methylene}-1-benzofuran-3(2H)-one

MS (m/z) 413.1 (MH−).

### Example 372 (2Z)-2-{[4-(4-Acetyl phenyl)-1-methyl-1H-indol-3-yl]methylene}-4,6-dihydroxy-1-benzofuran-3(2H)-one

MS (m/z) 426.4 (MH⁺).

### Example 370 (2Z)-2-{[4-(4-Aminophenyl)-1-methyl-1H-indol-3-yl]methylene}-4,6-dihydroxy-1-benzofuran-3(2H)-one

MS (m/z) 399.3 (MH⁺).

### Example 374 (2Z)-4,6-Dihydroxy-2-{[1-methyl-4-(3-thienyl)-1H-indol-3-yl]methylene}-1-benzofuran-3(2H)-one

MS (m/z) 388 (MH−.)

### Example 375 (2Z)-4,6-Dihydroxy-2-[(1-methyl-4-pyridin-3-yl-1H-indol-3-yl)methylene]-1-benzofuran-3(2H)-one

MS (m/z) 385.2 (MH⁺).

### Example 373 (2Z)-4,6-Dihydroxy-2-[(1-methyl-4-pyridin-4-yl-1H-indol-3-yl)methylene]-1-benzofuran-3(2H)-one

MS (m/z) 385.2 (MH⁺).

### Example 379 (2Z)-4,6-Dihydroxy-2-{[4-(4-hydroxyphenyl)-1-methyl-1H-indol-3-yl]methylene}-1-benzofuran-3(2H)-one

MS (m/z) 400.2 (MH⁺).

### Example 380 (2Z)-2-{[4-(6-Aminopyridin-3-yl)-1-methyl-1H-indol-3-yl]methylene}-4,6-dihydroxy-1-benzofuran-3(2H)-one

MS (m/z) 400.2 (MH⁺).

### Example 382 Ethyl 4-{3-[(Z)-(4,6-Dihydroxy-3-oxo-1-benzofuran-2(3H)-yl idene)methyl]-1-methyl-1H-indol-4-yl}benzoate

MS (m/z) 456.3 (MH⁺).

### Example 389 (2Z)-4,6-Dihydroxy-2-({1-methyl-4-[4-(methylamino)phenyl]-1H-indol-3-yl}methylene)-1-benzofuran-3(2H)-one

MS (m/z) 413.2 (MH⁺).

### Example 386 (2Z)-4,6-Dihydroxy-2-{[1-methyl-4-(6-morpholin-4-yl pyridin-3-yl)-1H-indol-3-yl]methylene}-1-benzofuran-3(2H)-one

MS (m/z) 470.2 (MH⁺).

### Example 385 (2Z)-2-({4-[4-(Dimethylamino)phenyl]-1-methyl-1H-indol-3-yl}methylene)-4,6-dihydroxy-1-benzofuran-3(2H)-one

MS (m/z) m/z 427.2 (MH⁺).

### Example 397 5-{3-[(Z)-(4,6-Dihydroxy-3-oxo-1-benzofuran-2(3H)-yl idene)methyl]-1-methyl-1H-indol-4-yl}pyridine-2-carbonitrile

MS (ESI) m/z 408.1 (MH−).

### Example 398 (2Z)-2-({4-[3-(Dimethylamino)phenyl]-1-methyl-1H-indol-3-yl}methylene)-4,6-dihydroxy-1-benzofuran-3(2H)-one

HRMS: calcd for C26H22N2O4+H⁺, 427.16523; found (ESI-FTMS, [M+H]⁺), 427.16507;

### Example 402 (2Z)-4,6-Dihydroxy-2-[(1-methyl-4-{6-[(2-morpholin-4-ylethyl)amino]pyridin-3-yl}-1H-indol-3-yl)methylene]-1-benzofuran-3(2H)-one

MS (ESI) m/z 513.3 (MH⁺).

### Example 403 (2Z)-4,6-Dihydroxy-2-({1-methyl-4-[1-(2-morpholin-4-ylethyl)-1H-pyrazol-4-yl]-1H-indol-3-yl}methylene)-1-benzofuran-3(2H)-one

MS (ESI) m/z 487.3 (MH⁺).

### Example 419 (2Z)-2-{[4-(2-Aminophenyl)-1-methyl-1H-indol-3-yl]methylene}-4,6-dihydroxy-1-benzofuran-3(2H)-one

MS (ESI) m/z 399.3 (MH⁺).

### Example 420 (2Z)-4,6-Dihydroxy-2-{[1-methyl-4-(4-nitrophenyl)-1H-indol-3-yl]methylene}-1-benzofuran-3(2H)-one

HRMS: calcd for C24H16N2O6+H⁺, 429.10811; found (ESI, [M+H]⁺ Obs′d), 429.1082;

### Example 376 Preparation of (2Z)-4,6-dihydroxy-2-{[4-(4-methoxyphenyl)-1-methyl-1H-indol-3-yl]methylene}benzofuran-3(2H)-one

To a mixture of 4-(4-methoxyphenyl)-1-methyl-1H-indole-3-carbaldehyde (132 mg, 0.498 mmol), 4,6-dihydroxybenzofuran-3(2H)-one (83 mg, 0.498 mmol) and 8 ml absolute ethanol was added one drop of concentrated hydrochloric acid. The reaction was heated to dissolve solids, solution turn a dark purple. This was heated at reflux for ½ hour then stirred 18 hours in an oil bath at 80° C. Reaction mixture was cooled and the solid collected on a sintered glass funnel washing with cold ethanol and air-dried. The dull yellow solid (2Z)-4,6-dihydroxy-2-{[4-(4-methoxyphenyl)-1-methyl-1H-indol-3-yl]methylene}benzofuran-3(2H)-one (145 mg, 0.351 mmol, 70.5% yield), mp 289-91 dec. MS (m/z): 412.2 (MH−).

### Example 439 Preparation of (2Z)-4,6-dihydroxy-2-{[1-methyl-4-(4-methylpiperazin-1-yl)-1H-indol-3-yl]methylene}-1-benzofuran-3(2H)-one

A mixture of 300 mg (0.78 mmol) of 2-[(4-bromo-1-methyl-1H-indol-3-yl)methylene]-4,6-dihydroxy-1-benzofuran-3(2H)-one, 0.4 mL (3.9 mmol) of 1-methylpiperazin, 107.3 mg (0.117 mmol) of tri(dibenzylidenacetone)dipalladium(0), tri-tert-butylphosphine 47.3 mg (0.234 mmol), and 150 mg (1.56 mmol) of sodium tert-butoxide, was placed in a microwave vial. To the mixture was added 4 mL of 1-methyl-2-pyrrolidinone. The sealed tube was heated by microwave for twenty minutes at 120° C. After cooling, the mixture was filtered through Celite and washed with 12% methanol in methylene chloride. After the solvent was evaporated, the residue was purified by column chromatography (1% Ammonium hydroxide: 14% methanol in methylene chloride) to give 80 mg of a yellow solid. The solid was further purified by reverse phase HPLC to give 24.5 mg of (2Z)-4,6-dihydroxy-2-{[1-methyl-4-(4-methylpiperazin-1-yl)-1H-indol-3-yl]methylene}-1-benzofuran-3(2H)-one as an orange yellow solid (8% yield). MS (m/z) 406.3 (MH⁺).

The next two steps for the following final compounds were prepared using the route for Example 381.

### Example 425 4-{3-[(Z)-(4,6-Dihydroxy-3-oxo-1-benzofuran-2(3H)-yl idene)methyl]-1-[2-(4-methylpiperazin-1-yl)ethyl]-1H-indol-4-yl}benzamide

HRMS: calcd for C₃₁H₃₀N₄O₅+H⁺, 539.22890; found (ESI, [M+H]⁺), 539.2287;

### Example 438 (2Z)-2-({4-[4-(Dimethylamino)phenyl]-1-[2-(4-methylpiperazin-1-yl)ethyl]-1H-indol-3-yl}methylene)-4,6-dihydroxy-1-benzofuran-3(2H)-one

MS (m/z) 539.4 (MH⁺).

### Example 371 Preparation of (2Z)-4,6-dihydroxy-2-{[4-(4-methoxyphenyl)-1H-indol-3-yl]methylene}benzofuran-3(2H)-one

To a mixture of 4-(4-methoxyphenyl)-1H-indole-3-carbaldehyde (130 mg, 0.497 mmol), 4,6-dihydroxybenzofuran-3(2H)-one (83 mg, 0.497 mmol) and 8 ml absolute ethanol was added one drop of concentrated hydrochloric acid. Reaction quickly turns dark purple. This was heated at reflux for ½ hour, stirred 18 hours in an oil bath at 80° C., then allowed to cool to room temperature. The solution was evaporated to dryness giving a very dark gum. When this was treated with CDCL₃ a solid formed which was filtered and washed with fresh CDCL₃. NMR of the chloroform filtrate showed no product. Solid does have product, but is not clean by NMR. The solid was mixed with 2:1 ethyl acetate/hexanes and passed through a short column of silica gel and eluted with the same solvent, the orange band was collected and evaporated. The gum was dissolved in a little acetonitrile. Water was added and the resulting orange solid was collect on a sintered glass funnel, washed with water and dried to give (2Z)-4,6-dihydroxy-2-{[4-(4-methoxyphenyl)-1H-indol-3-yl]methylene}benzofuran-3(2H)-one (56 mg, 0.140 mmol, 28.2% yield), MS (m/z) 400.2 (MH⁺).

### Example 409 Preparation of (2Z)-4,6-dimethoxy-2-[(1-methyl-2-phenyl-1H-indol-3-yl)methylene]benzofuran-3(2H)-one

To a mixture of 1-methyl-2-phenyl-1H-indole-3-carbaldehyde (471 mg, 2.002 mmol), 4,6-dimethoxybenzofuran-3(2H)-one (389 mg, 2.002 mmol) and ethanol (30 mL) was added 2 drops of concentrated hydrochloric acid. All solids dissolve to give a deep maroon solution, which slowly lightens and precipitates a solid, while heated by an oil bath at 80° C. Stirred overnight. Reaction mixture cooled and the solid collected washed with ethanol and air dried to give an orange brown solid, (2Z)-4,6-dimethoxy-2-[(1-methyl-2-phenyl-1H-indol-3-yl)methylene]benzofuran-3(2H)-one (699 mg, 1.699 mmol, 85% yield), mp 257-8. MS (m/z) 414.2 (MH⁺).

### Example 421 Preparation of (2Z)-4-hydroxy-6-methoxy-2-[(1-methyl-2-phenyl-1H-indol-3-yl)methylene]benzofuran-3(2H)-one

A mixture of (2Z)-4,6-dimethoxy-2-[(1-methyl-2-phenyl-1H-indol-3-yl)methylene]benzofuran-3(2H)-one (411 mg, 0.999 mmol) and dichloromethane (20 mL) was stirred and cooled in an ice bath, boron tribromide (1.199 mL, 1.199 mmol) was added. The mixture turns a deep purple. Stirred overnight. Reaction mixture cooled and the reaction quenched with ice and water the dark solid was dissolved in 15% methanol in chloroform and loaded onto silica gel and purified by chromatography on the ISCO Companion with a chloroform methanol gradient. The product peak (with correct MS) was collected, evaporated, triturated with 3:1 Hexanes/ethyl acetate, filtered, dried to give (2Z)-4-hydroxy-6-methoxy-2-[(1-methyl-2-phenyl-1H-indol-3-yl)methylene]benzofuran-3(2H)-one (184.2 mg, 0.463 mmol, 46.4% yield), mp 221-3. MS (m/z) 398.3 (MH⁺).

The following final compounds were prepared using route for Example 409.

### Example 408 (2Z)-2-[(4-Bromo-1-methyl-1H-indol-3-yl)methylene]-4,6-dimethoxybenzofuran-3(2H)-one

MS (m/z) 412.2 (MH⁺).

### Example 423 (2Z)-7-Bromo-4-methoxy-2-[(1-methyl-4-phenyl-1H-indol-3-yl)methylene]benzofuran-3(2H)-one

MS (m/z) 460.2 (MH⁺).

### Example 432 (2Z)-6-Hydroxy-4-methoxy-2-[(1-methyl-2-phenyl-1H-indol-3-yl)methylene]benzofuran-3(2H)-one

MS (m/z) 398.3 (MH⁺).

### Example 441 (2Z)-4-Hydroxy-2-[(1-methyl-1H-indol-3-yl)methylene]benzofuran-3(2H)-one

MS (m/z) 292.2 (MH⁺).

### Example 442 (2Z)-4-Hydroxy-2-[(1-methyl-2-phenyl-1H-indol-3-yl)methylene]benzofuran-3(2H)-one

MS (m/z) 368.2 (MH⁺).

### Example 443 (2Z)-6-Hydroxy-2-((1-methyl-4-phenyl-1H-indol-3-yl)methylene)benzofuran-3(2H)-one

MS (m/z) 368.2 (MH⁺).

The following final compounds were prepared using route for Example 421.

### Example 428 (2Z)-7-Bromo-4-hydroxy-2-[(1-methyl-4-phenyl-1H-indol-3-yl)methylene]benzofuran-3(2H)-one

MS (m/z) 446.2 (MH⁺).

### Example 437 Preparation of 3-[(Z)-(4,6-dihydroxy-3-oxo-1-benzofuran-2(3H)-yl idene)methyl]-1-methyl-2-phenyl-1H-indole-4-carbonitrile

A mixture of 3-formyl-1-methyl-2-phenyl-1H-indole-4-carbonitrile (128 mg, 0.49 mmol), 4,6-dihydroxy-benzofuran-3-one (82 mg, 0.49 mmol) and one drop of concentrated HCl was heated to 80° C. for 3 hours. The reaction was cooled and concentrated. The rust colored residue was stirred in acetone, filtered and dried in vacuo to afford 95 mg (0.23 mmol, 47%) of 3-[(Z)-(4,6-dihydroxy-3-oxo-1-benzofuran-2(3H)-ylidene)methyl]-1-methyl-2-phenyl-1H-indole-4-carbonitrile. mp: decomposes at 325° C., MS (m/z) 409.3 (MH⁺).

### Example 413 Preparation of (2Z)-6-bromo-2-({1-[3-(dimethylamino)propyl]-5-methoxy-1H-indol-3-yl}methylene)-1-benzofuran-3(2H)-one

A mixture of 1-[3-(dimethylamino)propyl]-5-methoxy-1H-indole-3-carbaldehyde (416 mg, 1.6 mmol), 6-bromo-1-benzofuran-3(2H)-one (1.53 g, 7.2 mmol), and ammonium chloride (1g) in 20 mL of ethanol was heated at reflux for 20 hours. The mixture was cooled to room temperature and the precipitates were collected by filtration. The solids thus obtained were washed with water, dried, then washed with ethyl acetate, and dried. The desired (2Z)-6-bromo-2-({1-[3-(dimethylamino)propyl]-5-methoxy-1H-indol-3-yl}methylene)-1-benzofuran-3(2H)-one was obtained as orange solids (506 mg). MS (m/z) 455.2 (MH⁺).

### Example 417 Preparation of tert-butyl (2Z)-[2-({1-[3-(dimethylamino)propyl]-5-methoxy-1H-indol-3-yl}methylene)-3-oxo-2,3-dihydro-1-benzofuran-6-yl]carbamate

A mixture of (2Z)-6-bromo-2-({1-[3-(dimethylamino)propyl]-5-methoxy-1H-indol-3-yl}methylene)-1-benzofuran-3(2H)-one (120 mg, 0.26 mmol), tert-butylcarbamate (800 mg, 6.8 mmol), t-BuONa (100 mg, 1.04 mmol), Pd(OAc)₂ (58 mg, 0.26 mmol), and Xant Phos (150 mg, 0.26 mmol) in 15 mL of 1,4-dioxane was stirred at room temperature for 14 hr. The resulting reaction mixture was diluted with ethyl acetate, washed with saturated NaHCO₃ aqueous solution and saturated NaCl aqueous solution, dried over MgSO₄, filtered, concentrated, and purified by chromatography over a 40 g silica column, eluting with 5% methanol in dichloromethane to provide 100.1 mg of tert-butyl (2Z)-[2-({1-[3-(dimethylamino)propyl]-5-methoxy-1H-indol-3-yl}methylene)-3-oxo-2,3-dihydro-1-benzofuran-6-yl]carbamate as a yellow solid. MS (m/z) 492.4 (MH⁺).

### Example 418 Preparation of (2Z)-6-amino-2-({1-[3-(dimethylamino)propyl]-5-methoxy-1H-indol-3-yl}methylene)-1-benzofuran-3(2H)-one

To a solution of tert-butyl (2Z)-[2-({1-[3-(dimethylamino)propyl]-5-methoxy-1H-indol-3-yl}methylene)-3-oxo-2,3-dihydro-1-benzofuran-6-yl]carbamate (50 mg, 0.10 mmol) in 10 mL of dichloromethane was added 4N HCl in 1,4-dioxane (300 μL, 1.2 mmol). The solution mixture was stirred at room temperature for 4 hours and filtered. The obtained solid was purified by chromatography over a 40 g silica column, eluting with 10% methanol in dichloromethane to provide 18 mg of (2Z)-6-amino-2-({1-[3-(dimethylamino)propyl]-5-methoxy-1H-indol-3-yl}methylene)-1-benzofuran-3(2H)-one as a yellow solid. MS (m/z) 392.3 (MH⁺).

The following final compounds were prepared using route for Example 417.

### Example 405 Methyl [2-({1-[3-(dimethylamino)propyl]-5-methoxy-1H-indol-3-yl}methylene)-3-oxo-2,3-dihydro-1-benzofuran-6-yl]carbamate

MS (m/z) 450.4 (MH⁺).

### Example 406 1-(2Z)-[2-({1-[3-(Dimethylamino)propyl]-5-methoxy-1H-indol-3-yl}methylene)-3-oxo-2,3-dihydro-1-benzofuran-6-yl]-3-methyl urea

MS (m/z) 449.3 (MH⁺).

### Example 407 N-(2Z)-[2-({1-[3-(Dimethylamino)propyl]-5-methoxy-1H-indol-3-yl}methylene)-3-oxo-2,3-dihydro-1-benzofuran-6-yl]acetamide

MS (m/z) 434.3 (MH⁺).

### Example 422 N-(2Z)-[2-({1-[3-(Dimethylamino)propyl]-5-methoxy-1H-indol-3-yl}methylene)-3-oxo-2,3-dihydro-1-benzofuran-6-yl]methanesulfonamide

MS (m/z) 470.3 (MH⁺).

### Example 424 Preparation of (2Z)-2-({1-[3-(dimethylamino)propyl]-5-methoxy-1H-indol-3-yl}methylene)-6-(hydroxymethyl)-1-benzofuran-3(2H)-one

A mixture of (2Z)-6-bromo-2-({1-[3-(dimethylamino)propyl]-5-methoxy-1H-indol-3-yl}methylene)-1-benzofuran-3(2H)-one (20 mg, 0.044 mmol), hydroxymethyltributyltin (141 mg, 0.44 mmol, prepared by using the procedure from Organic Syntheses, 1993, 71, 133), Pd (PPh₃)₄ (5 mg, 10 mol %) and 1.5 mL of 1,4-dioxane was heated in the microwave at 90° C. for 5 minutes. After cooling down, the reaction mixture was diluted with ethyl acetate, washed with H₂O and brine solution, dried over MgSO₄, filtered, concentrated, and purified by chromatography over silica gel eluting with 5% methanol in dichloromethane to give (2Z)-2-({1-[3-(dimethylamino)propyl]-5-methoxy-1H-indol-3-yl}methylene)-6-(hydroxymethyl)-1-benzofuran-3(2H)-one as an orange solid. Yield: 6 mg (35%). MS (m/z): 407.2 (MH⁺).

### Example 400 (2Z)-4-Hydroxy-2-[(1-methyl-4-phenyl-1H-indol-3-yl)methylene]-1-benzofuran-3(2H)-one

A mixture of 1-methyl-4-phenyl-1H-indole-3-carbaldehyde, 4,6-dihydroxycoumaranone, ethanol, and conc. HCl was heated. After heating, the precipitate was filtered and washed with ethanol to yield (2Z)-4-Hydroxy-2-[(1-methyl-4-phenyl-1H-indol-3-yl)methylene]-1-benzofuran-3(2H)-one, MS (m/z) 368.3 (MH⁺).

Procedure to make 1-methyl-3-(3-oxo-2,3-dihydro-1-benzofuran-5-yl)urea:

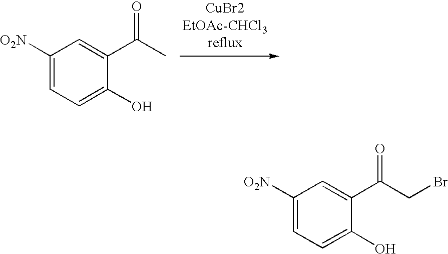

Reference: J. Org. Chem. 1964, 29, 3459

A solution of 2′-hydroxy-5′-nitroacetophenone (5.03 g, 28 mmol) in CHCl₃ (45 mL) was added to a stirred mixture of CuBr₂ (15.13 g, 68 mmol, ground in a mortar-pestle) in EtOAc (45 mL) near reflux. Resulting mixture stirred vigorously at reflux under N₂ (balloon) for 3 hours, then cooled to room temperature. Reaction mixture suction filtered through paper and filtrate concentrated to give a solid that was triturated with 15% EtOAc:Hexanes (2×100 mL) and filtered. The washings were collected and concentrated and the resulting residue washed with 10% EtOAc-Hexanes (3×25 mL) leaving another crop of solid. The 2 solids obtained were combined, dissolved in CHCl₃ and suction filtered through paper. The filtrate was concentrated to give 2-bromo-1-(2-hydroxy-5-nitrophenyl)ethanone as an off-white solid, 4.74 g, 65% yield.

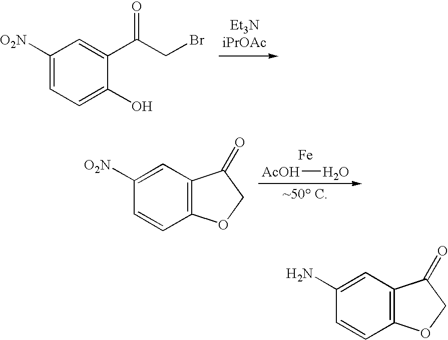

To a stirred solution of 2-bromo-1-(2-hydroxy-5-nitrophenyl)ethanone (4.74 g, 18 mmol) in isopropyl acetate (120 mL) was added triethylamine (2.53 mL, 19 mmol) at room temperature. Resulting mixture stirred for 90 minutes and then suction filtered through paper. The filtrate was concentrated and the crude product dissolved in EtOAc (60 mL) and used directly in the iron-mediated nitro reduction. A mixture of iron powder (5.02 g, 90 mmol, −325 mesh) in AcOH (25 mL) and H₂O (5 mL) stirred vigorously at 50° C. (oil bath) for 15 minutes. The flask was removed from the oil bath and additional H₂O (20 mL) added. To the warm, stirred mixture was added a solution of fresh 5-nitro-1-benzofuran-3(2H)-one in EtOAc in portions (˜2 mL portions) over a period of 20-25 minutes to maintain a slight exotherm. After addition was complete, the reaction mixture stirred for 5 minutes. H₂O (25 mL) was added, followed by EtOAc (150 mL). The mixture stirred vigorously for 10 seconds then EtOAc layer decanted off into aqueous Na₂CO₃ (46 g in 200 mL). Reaction mixture extracted further with EtOAc (6×50 mL) by stirring vigorously for 10 seconds then decanting into aqueous Na₂CO₃. Aqueous Na₂CO₃ layer extracted with EtOAc (100 mL). EtOAc extracts combined, washed with saturated aqueous NaCl (100 mL), dried over Na₂SO₄, decanted, and concentrated. Crude product immediately purified by SiO₂ chromatography using 20% EtOAc-CH₂Cl₂ to give the desired 5-amino-1-benzofuran-3(2H)-one, 2.25 g, 84% (2-steps) as a yellow solid.

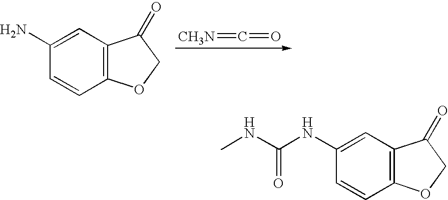

To a solution of 5-amino-1-benzofuran-3(2H)-one (450 mg, 3.0 mmol) in 50 mL of tetrahydrofuran was added methyl isocyanate (1M in toluene, 15 mL, 15 mmol). The mixture was stirred at room temperature for 3 days and filtered. The desired 1-methyl-3-(3-oxo-2,3-dihydro-1-benzofuran-5-yl)urea was obtained as a tan solid, 460 mg, 74% yield. MS: m/z 205.1 (MH−).

Preparation of methyl isocyanate: To a suspension of sodium azide (450 mg, 6.9 mmol) in 6.5 mL of toluene at 0° C. is added acetyl chloride (500 mg, 6.3 mmol). The mixture is refluxed with dry ice-acetone condenser cooling under nitrogen for 6 hrs, and cooled to room temperature. The supernatant is decanted, and used as 1.0 M methyl isocyanate solution in toluene.

### Suzuki-Coupling Procedure:

A mixture of the 3-formyl-2-bromoindole, boronic acid/ester (1-2 eq), Pd(OAc)₂ (3-5 mol %), PPh₃ (9-15 mol %) and K₃PO₄ (3 eq) in 1,2-dimethoxyethane and water was subjected to microwave conditions (155° C.). Reaction mixture cooled to room temperature, poured into water and extracted with EtOAc. EtOAc extracts combined, washed with saturated aqueous NaCl (25 mL), dried over Na₂SO₄, and concentrated. The mixture was purified by silica gel column chromatography.

### (Z)-1-(2-((5-Methoxy-2-(1,3,5-trimethyl-1H-pyrazol-4-yl)-1H-indol-3-yl)methylene)-3-oxo-2,3-dihydrobenzofuran-5-yl)-3-methylurea MS (m/z): 472.2 (MH⁺)

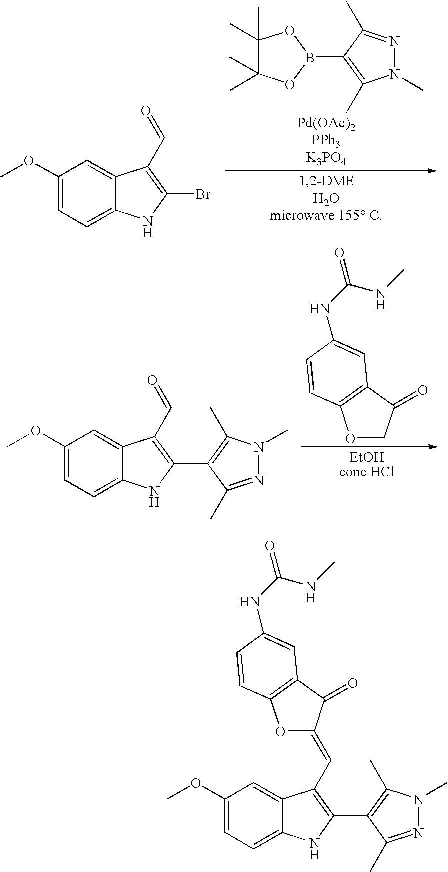

### Preparation of 5-methoxy-2-(1,3,5-trimethyl-1H-pyrazol-4-yl)-1H-indole-3-carbaldehyde

A mixture of 2-bromo-5-methoxy-1H-indole-3-carbaldehyde (132 mg, 0.52 mmol), 1,3,5-trimethyl-1-H-pyrazole-4-boronic acid pinacol ester (184 mg, 0.78 mmol), Pd(OAc)₂ (7 mg, 0.03 mmol), PPh₃ (24 mg, 0.09 mmol) and K₃PO₄ (331 mg, 1.56 mmol) in 1,2-dimethoxyethane (1.5 mL) and water (1.2 mL) was subjected to microwave conditions (155° C., 40 min). Reaction mixture cooled to room temperature, poured into water (25 mL) and extracted with EtOAc (2×50 mL). EtOAc extracts combined, washed with saturated aqueous NaCl (25 mL), dried over Na₂SO₄, and concentrated. The mixture purified by silica gel column chromatography (eluent: 80% EtOAc-hexanes). Yield >100%. MS (m/z): 284.2 (MH⁺).

### Preparation of (Z)-1-(2-((5-methoxy-2-(1,3,5-trimethyl-1H-pyrazol-4-yl)-1H-indol-3-yl)methylene)-3-oxo-2,3-dihydrobenzofuran-5-yl)-3-methylurea

Concentrated aqueous HCl (2 drops) was added to a stirred mixture of 5-methoxy-2-(1,3,5-trimethyl-1H-pyrazol-4-yl)-1H-indole-3-carbaldehyde (68 mg, 0.24 mmol) and 1-methyl-3-(3-oxo-2,3-dihydrobenzofuran-5-yl)urea (60 mg, 0.29 mmol) in EtOH (1.5 mL). Resulting mixture stirred at room temperature for 5 hours. EtOAc (2 mL) added and mixture filtered. Filtrate collected and concentrated. Crude product dissolved in EtOH (5 mL) and treated with saturated aqueous Na₂CO₃ (2 mL) and resulting mixture stirred at 75° C. for 15 minutes. The mixture cooled to room temperature, EtOAc (10 mL) added, organic layer collected and concentrated. Residue dissolved in EtOH (5 mL) and triturated with EtOAc (3 mL) then filtered. Filtrate concentrated and purified by preparative HPLC. Yield 35%. MS (m/z): 472.2 (MH⁺).

### (Z)-1-(2-((5-Methoxy-2-(1,3,5-trimethyl-1H-pyrazol-4-yl)-1H-indol-3-yl)methylene)-3-oxo-2,3-dihydrobenzofuran-5-yl)-3-(pyridin-3-yl)urea MS (m/z): 535.2 (MH⁺)

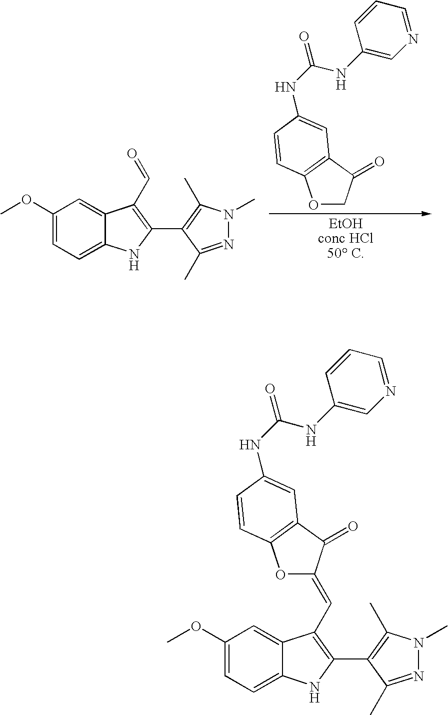

### (Z)-1-(2-((2-Bromo-5-methoxy-1H-indol-3-yl)methylene)-3-oxo-2,3-dihydrobenzofuran-5-yl)-3-methylurea MS (m/z): 442.0 (MH⁺)

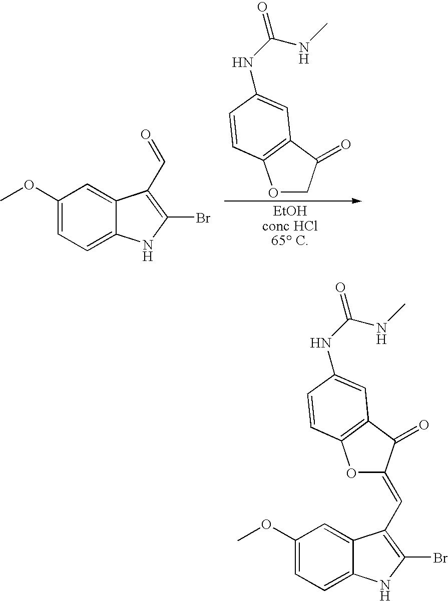

### Preparation of (Z)-1-(2-((7-fluoro-5-methoxy-2-(1,3,5-trimethyl-1H-pyrazol-4-yl)-1H-indol-3-yl)methylene)-3-oxo-2,3-dihydrobenzofuran-5-yl)-3-methylurea

### 3-Formyl-2-bromoindoles via Gassman oxindole-Vilsmeier-Haack reactions

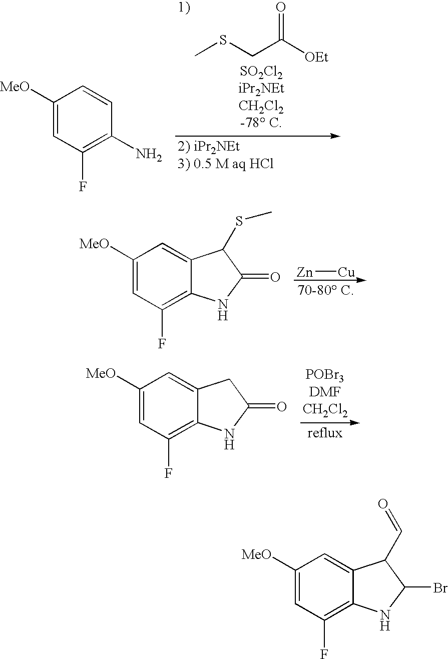

### Preparation of 7-fluoro-5-methoxy-3-(methylthio)indolin-2-one

Method similar to that referenced in J. Med. Chem. 2001, 44, 4339.

Sulfuryl chloride (3.05 mL, 38 mmol) added to a stirred solution of ethyl methylthioacetate (5.15 mL, 40 mmol) in CH₂Cl₂ (60 mL) at −78° C. over a period of 5 minutes. Resulting mixture stirred at −78° C. for 15 minutes then a solution of 2-fluoro-4-methoxyaniline (5.40 g, 38 mmol) and iPr₂NEt (6.62 mL, 38 mmol) in CH₂Cl₂ (60 mL) was added over a period of 45 minutes. Resulting mixture stirred at −78° C. for 30 minutes then iPr₂NEt (6.62 mL, 38 mmol) added over a period of 4 minutes. Cooling bath removed, mixture stirred overnight, and then solvent removed. Crude product dissolved in EtOAc (150 mL), 0.5 M aqueous HCl (150 mL) added, and the resulting mixture stirred overnight. Organic layer collected and aqueous layer extracted with EtOAc (2×150 mL). Organic layers combined, washed with water (50 mL), then saturated aqueous NaCl (2×50 mL), dried over Na₂SO₄ and concentrated. Resulting solid washed with 30% EtOAc-hexanes and then dried in vacuo. Yield 50%. MS (m/z): 226.1 (MH−).

### Preparation of 7-fluoro-5-methoxyindolin-2-one

A mixture of 7-fluoro-5-methoxy-3-(methylthio)indolin-2-one (4.35 g, 19 mmol) and zinc-copper couple (3.50 g) in AcOH (25 mL) and EtOAc (25 mL) was stirred at 70° C. for 2 hours, then overnight at 60° C. The mixture cooled to room temperature, diluted with EtOAc (100 mL) and suction filtered. Filtrate concentrated. Yield >100%. MS (m/z): 182.0 (MH⁺).

### Preparation of 2-bromo-7-fluoro-5-methoxy-1H-indole-3-carbaldehyde

A solution of POBr₃ (12.53 g, 44 mmol) in CH₂Cl₂ (50 mL) was added to a stirred solution of DMF (4.36 mL, 56 mmol) and CH₂Cl₂ (50 mL) over a period of 10 minutes. Resulting mixture stirred at reflux for 10 minutes and then a mixture of 7-fluoro-5-methoxyindolin-2-one (3.46 g, 19 mmol) in CH₂Cl₂ (30 mL) added over a period of 3 minutes. Resulting mixture stirred at reflux for 1 hour, cooled to room temperature and filtered. Filter cake rinsed with CH₂Cl₂ (2×50 mL) then the filter cake added to water (150 mL). The mixture swirled for 30 seconds, sat for 1 hour, and then extracted with EtOAc (4×100 mL). EtOAc extracts combined, washed with saturated aqueous NaCl (2×50 mL), dried over Na₂SO₄, and concentrated to give a solid. After sitting overnight, additional solid obtained from the aqueous layer by filtration and subsequent washing with water (3×25 mL). Solids combined and dried in vacuo. Yield 74%. MS (m/z): 271.9 (MH⁺).

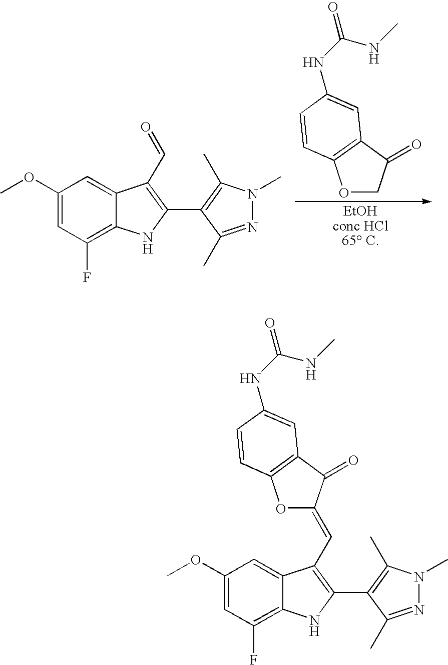

### Preparation of 7-fluoro-5-methoxy-2-(1,3,5-trimethyl-1H-pyrazol-4-yl)-1H-indole-3-carbaldehyde

A mixture of 2-bromo-7-fluoro-5-methoxy-1H-indole-3-carbaldehyde (269 mg, 0.99 mmol), 1,3,5-trimethyl-1-H-pyrazole-4-boronic acid pinacol ester (281 mg, 1.19 mmol), diacetoxy palladium (7 mg, 0.03 mmol), triphenylphosphine (24 mg, 0.09 mmol), and potassium phosphate (630 mg, 2.97 mmol) were treated with 1,2-dimethoxyethane (2.0 mL) and water (1.5 mL) then subjected to microwave conditions (155° C., 30 min). The mixture cooled to room temperature, diluted with water (25 mL) and extracted with EtOAc (3×50 mL). EtOAc extracts combined and washed with saturated aqueous NaCl (25 mL), dried over Na₂SO₄ and concentrated. Purified by silica gel chromatography (eluent: 80-100% EtOAc-hexanes gradient). Yield 75%. MS (m/z): 302.1 (MH⁺).

### Preparation of (Z)-1-(2-((7-fluoro-5-methoxy-2-(1,3,5-trimethyl-1H-pyrazol-4-yl)-1H-indol-3-yl)methylene)-3-oxo-2,3-dihydrobenzofuran-5-yl)-3-methylurea

Concentrated aqueous HCl (4 drops) was added to a stirred mixture of 7-fluoro-5-methoxy-2-(1,3,5-trimethyl-1H-pyrazol-4-yl)-1H-indole-3-carbaldehyde (91 mg, 0.30 mmol) and 1-methyl-3-(3-oxo-2,3-dihydrobenzofuran-5-yl)urea (74 mg, 0.36 mmol) in EtOH (1.5 mL). Resulting mixture stirred at 65° C. for 3 hours, and then overnight at 60° C. The mixture cooled to room temperature and treated with saturated aqueous Na₂CO₃ (3 mL) and then stirred at 70° C. for 35 minutes. The mixture cooled to room temperature, diluted with EtOH (10 mL) and then filtered. Solid washed with water (3×5 mL) and EtOH (3 mL) and then dried in vacuo. Yield: 47%. MS (m/z): 490.2 (MH⁺).

### (Z)-1-(2-((2-(3,5-Dimethyl-1H-pyrazol-4-yl)-5-methoxy-1H-indol-3-yl)methylene)-3-oxo-2,3-dihydrobenzofuran-5-yl)-3-methylurea MS (m/z): 458.2 (MH⁺)

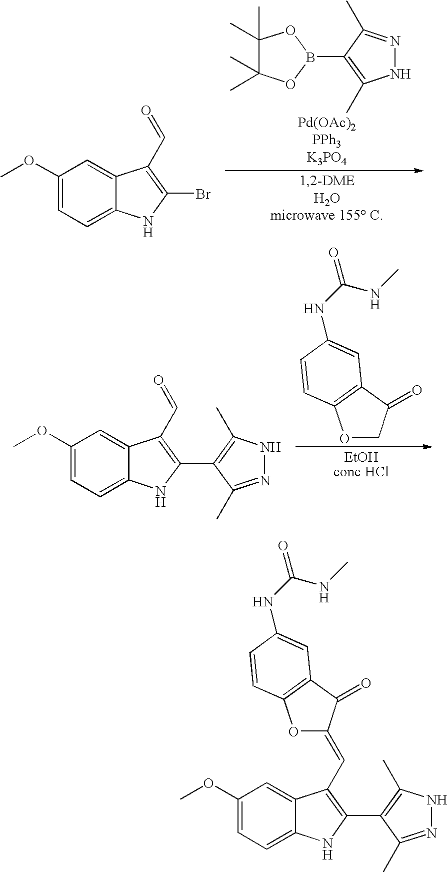

### Preparation of 2-(3,5-dimethyl-1H-pyrazol-4-yl)-5-methoxy-1H-indole-3-carbaldehyde

A mixture of 2-bromo-5-methoxy-1H-indole-3-carbaldehyde (129 mg, 0.51 mmol), 3,5-dimethylpyrazole-4-boronic acid pinacol ester (171 mg, 0.77 mmol), Pd(OAc)₂ (6 mg, 0.03 mmol), PPh₃ (31 mg, 0.12 mmol) and K₃PO₄ (325 mg, 1.53 mmol) in 1,2-dimethoxyethane (1.5 mL) and water (1 mL) was subjected to microwave conditions (155° C., 40 min). Additional Pd(OAc)₂ (6 mg, 0.03 mmol) and PPh₃ (30 mg, 0.12 mmol) added and reaction mixture re-subjected to microwave conditions (155° C., 50 min). Reaction mixture cooled to room temperature, poured into water (25 mL) and extracted with EtOAc (3×40 mL). EtOAc extracts combined, washed with saturated aqueous NaCl (25 mL), dried over Na₂SO₄, and concentrated. The mixture purified by silica gel column chromatography (eluent: 80-100% EtOAc-hexanes gradient). Yield 82%. MS (m/z): 270.2 (MH⁺).

### (Z)-1-(2-((2-(2,6-Dimethoxyphenyl)-5-methoxy-1H-indol-3-yl)methylene)-3-oxo-2,3-dihydrobenzofuran-5-yl)-3-methylurea MS (m/z): 500.2 (MH⁺)

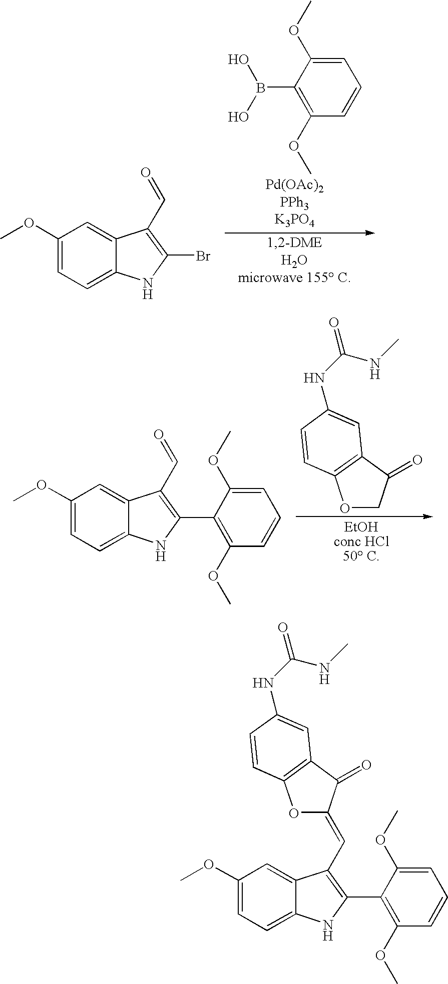

### Preparation of 2-(2,6-dimethoxyphenyl)-5-methoxy-1H-indole-3-carbaldehyde

A mixture of 2-bromo-5-methoxy-1H-indole-3-carbaldehyde (156 mg, 0.61 mmol), 2,6-dimethoxyphenyl boronic acid (133 mg, 0.73 mmol), Pd(OAc)₂ (4 mg, 0.02 mmol), PPh₃ (16 mg, 0.06 mmol), and K₃PO₄ (388 mg, 1.83 mmol) in 1,2-dimethoxyethane (1.5 mL) and water (1 mL) was subjected to microwave conditions (155° C., 30 min). Additional dimethoxyphenyl boronic acid (30 mg, 0.16 mmol) added and mixture re-subjected to microwave conditions (155° C., 15 min). Reaction mixture cooled to room temperature and diluted with EtOAc (5 mL). Organic layer collected, diluted with EtOAc (50 mL) and washed with saturated aqueous NaCl (25 mL), dried over Na₂SO₄ and concentrated. The mixture purified by silica gel column chromatography (eluent: 40-50% EtOAc-hexanes gradient). Yield 88%. MS (m/z): 312.1 (MH⁺).

### (Z)-1-(2-((2-(1-Isobutyl-1H-pyrazol-4-yl)-5-methoxy-1H-indol-3-yl)methylene)-3-oxo-2,3-dihydrobenzofuran-5-yl)-3-methylurea MS (m/z): 486.2 (MH⁺)

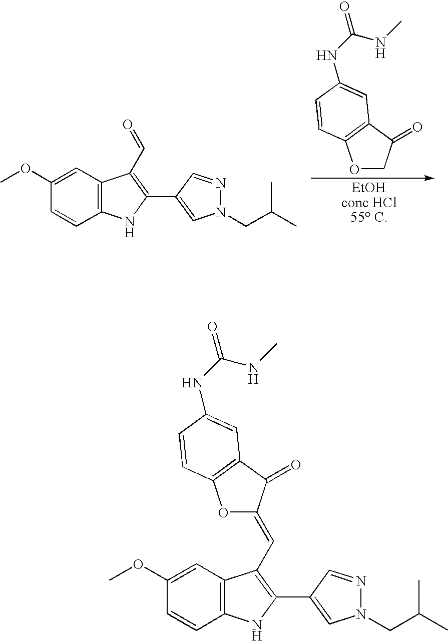

### Preparation of 2-(1-isobutyl-1H-pyrazol-4-yl)-5-methoxy-1H-indole-3-carbaldehyde

A mixture of 2-bromo-5-methoxy-1H-indole-3-carbaldehyde (206 mg, 0.81 mmol), 1-isobutyl-4-(4,4,5,5-tetramethyl-1,3,2-dioxaborolan-2-yl)-1H-pyrazole (243 mg, 0.97 mmol), diacetoxy palladium (7 mg, 0.03 mmol), triphenylphosphine (26 mg, 0.10 mmol), and potassium phosphate (516 mg, 2.43 mmol) were treated with DME (1.8 mL) and water (1.2 mL) then subjected to microwave conditions (155° C.) for 35 minutes. The mixture cooled to room temperature, poured into water (25 mL) and extracted with EtOAc (3×50 mL). EtOAc extracts combined and washed with saturated aqueous NaCl (25 mL), dried over Na₂SO₄ and concentrated. Purified by silica gel chromatography (eluent: 40-50% EtOAc-hexanes gradient). Yield 83%.

### (Z)-1-(2-((2-(1,3-Dimethyl-1H-pyrazol-4-yl)-5-methoxy-1H-indol-3-yl)methylene)-3-oxo-2,3-dihydrobenzofuran-5-yl)-3-methylurea MS (m/z): 458.2 (MH⁺)

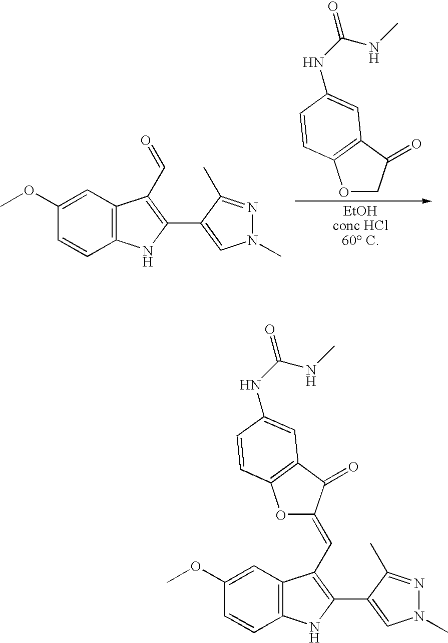

### Preparation of 2-(1,3-dimethyl-1H-pyrazol-4-yl)-5-methoxy-1H-indole-3-carbaldehyde

A mixture of 2-bromo-5-methoxy-1H-indole-3-carbaldehyde (315 mg, 1.24 mmol), 1,3-dimethyl-4-(4,4,5,5-tetramethyl-1,3,2-dioxaborolan-2-yl)-1H-pyrazole (303 mg, 1.36 mmol), diacetoxy palladium (9 mg, 0.04 mmol), triphenylphosphine (29 mg, 0.11 mmol), and potassium phosphate (789 mg, 3.72 mmol) in DME (2.5 mL) and water (1.5 mL) was subjected to microwave conditions (155° C., 30 min). Organic layer collected and concentrated. Residue purified by silica gel chromatography (eluent: 90% EtOAc-hexanes to 100% EtOAc gradient). Yield: 34%. MS (m/z): 270.1 (MH⁺).

### (Z)-1-(2-((2-(1,5-Dimethyl-1H-pyrazol-4-yl)-5-methoxy-1H-indol-3-yl)methylene)-3-oxo-2,3-dihydrobenzofuran-5-yl)-3-methylurea MS (m/z): 458.2 (MH⁺)

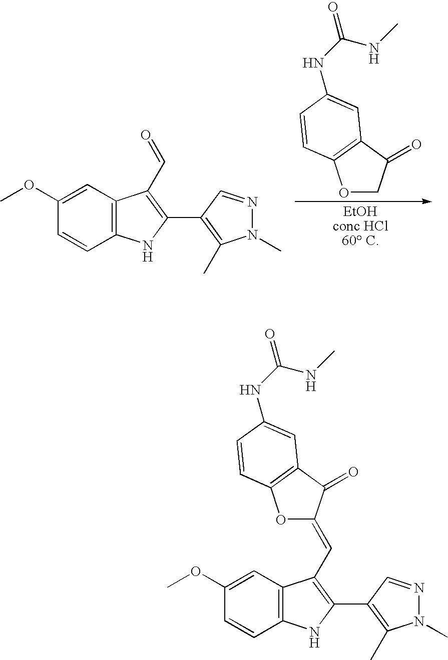

### Preparation of 2-(1,5-dimethyl-1H-pyrazol-4-yl)-5-methoxy-1H-indole-3-carbaldehyde

A mixture of 2-bromo-5-methoxy-1H-indole-3-carbaldehyde (315 mg, 1.24 mmol), 1,5-dimethyl-4-(4,4,5,5-tetramethyl-1,3,2-dioxaborolan-2-yl)-1H-pyrazole (303 mg, 1.36 mmol), diacetoxy palladium (9 mg, 0.04 mmol), triphenylphosphine (29 mg, 0.11 mmol), and potassium phosphate (789 mg, 3.72 mmol) in DME (2.5 mL) and water (1.5 mL) was subjected to microwave conditions (155° C., 30 min). Organic layer collected and concentrated. Residue purified by silica gel chromatography (eluent: 90% EtOAc-hexanes to 100% EtOAc gradient). Yield: 29%. MS (m/z): 270.1 (MH⁺).

### (Z)-1-(2-((5-Methoxy-2-(1-methyl-4-(trifluoromethyl)-1H-pyrazol-3-yl)-1H-indol-3-yl)methylene)-3-oxo-2,3-dihydrobenzofuran-5-yl)-3-methylurea MS (m/z): 512.2 (MH⁺)

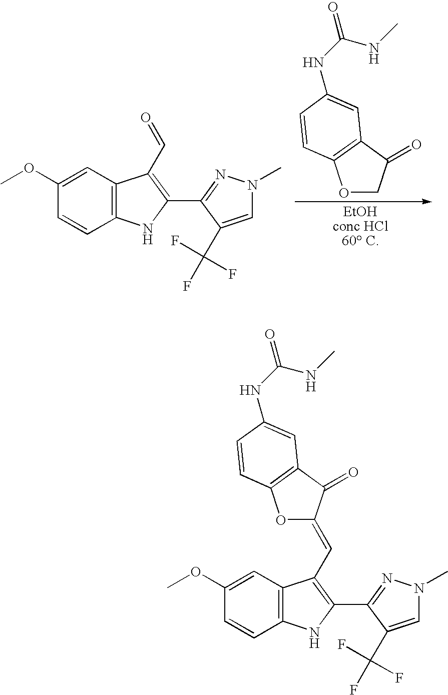

### Preparation of 5-methoxy-2-(1-methyl-4-(trifluoromethyl)-1H-pyrazol-3-yl)-1H-indole-3-carbaldehyde

A mixture of 2-bromo-5-methoxy-1H-indole-3-carbaldehyde (315 mg, 1.24 mmol), 1-methyl-3-(4,4,5,5-tetramethyl-1,3,2-dioxaborolan-2-yl)-4-(trifluoromethyl)-1H-pyrazole (376 mg, 1.36 mmol), diacetoxy palladium (9 mg, 0.040 mmol), triphenylphosphine (29 mg, 0.11 mmol), and potassium phosphate (789 mg, 3.72 mmol) in DME (2.5 mL) and water (1.5 mL) was subjected to microwave conditions (155° C., 30 min). EtOAc added (2 mL) to the cooled mixture and the organic layer collected and concentrated. Residue purified by silica gel chromatography (eluent: 30-35% EtOAc-hexanes gradient). Yield: 28%. MS (m/z): 324.1 (MH⁺).

### Preparation of (Z)-1-(2-((5-methoxy-2-(1-methyl-4-(trifluoromethyl)-1H-pyrazol-3-yl)-1H-indol-3-yl)methylene)-3-oxo-2,3-dihydrobenzofuran-5-yl)-3-methylurea

Concentrated aqueous HCl (6 drops) was added to a stirred mixture of 5-methoxy-2-(1-methyl-4-(trifluoromethyl)-1H-pyrazol-3-yl)-1H-indole-3-carbaldehyde (108 mg, 0.33 mmol) and 1-methyl-3-(3-oxo-2,3-dihydrobenzofuran-5-yl)urea (69 mg, 0.33 mmol) in EtOH (2 mL). Resulting mixture stirred at 60° C. for 5 hours, then cooled to room temperature. The reaction mixture diluted with EtOH (10 mL) and then saturated aqueous Na₂CO₃ added (2 mL). Resulting mixture stirred for 5 minutes then filtered and the filtrate concentrated. Residue purified by silica gel chromatography (eluent: 70-100% EtOAc-hexanes gradient). Yield: 22%. MS (m/z): 512.2 (MH⁺).

### (Z)-1-(2-((5-Methoxy-2-(1-(2-methoxyethyl)-3,5-dimethyl-1H-pyrazol-4-yl)-1H-indol-3-yl)methylene)-3-oxo-2,3-dihydrobenzofuran-5-yl)-3-methylurea MS (m/z): 516.2 (MH⁺)

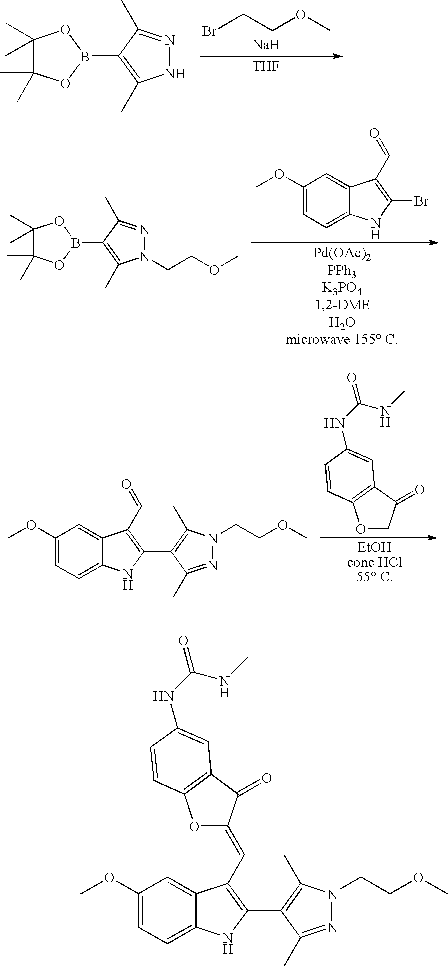

### Preparation of 1-(2-methoxyethyl)-3,5-dimethyl-4-(4,4,5,5-tetramethyl-1,3,2-dioxaborolan-2-yl)-1H-pyrazole

Sodium hydride (39 mg, 1.63 mmol) was added to a stirred solution of 3,5-dimethyl-4-(4,4,5,5-tetramethyl-1,3,2-dioxaborolan-2-yl)-1H-pyrazole (302 mg, 1.36 mmol) in THF (9.07 mL). Resulting mixture stirred for 5 minutes then 1-bromo-2-methoxyethane (153 μL, 1.63 mmol) added. Resulting mixture stirred for 30 minutes at room temperature then stirred overnight at 60° C. Additional NaH (˜50 mg) and 1-bromo-2-methoxyethane added (excess, 0.5 mL) and mixture heated to 68° C. for 3 hours. The reaction mixture cooled to room temperature, poured into H₂O (25 mL) and extracted with EtOAc (3×25 mL). EtOAc extracts combined, washed with saturated aqueous NaCl (25 mL), dried over Na₂SO₄, and concentrated. Crude product dissolved in hexanes (10 mL) and mixture sat for 15 minutes, filtered, and the filtrate collected and concentrated. Product used immediately.

### Preparation of 5-methoxy-2-(1-(2-methoxyethyl)-3,5-dimethyl-1H-pyrazol-4-yl)-1H-indole-3-carbaldehyde

A mixture of 2-bromo-5-methoxy-1H-indole-3-carbaldehyde (185 mg, 0.72 mmol), 1-(2-methoxyethyl)-3,5-dimethyl-4-(4,4,5,5-tetramethyl-1,3,2-dioxaborolan-2-yl)-1H-pyrazole (225 mg, 0.80 mmol), diacetoxy palladium (7 mg, 0.03 mmol), triphenylphosphine (23 mg, 0.09 mmol), and potassium phosphate (464 mg, 2.18 mmol) in DME (1.5 mL) and water (1 mL) was subjected to microwave conditions (155° C.) for 35 minutes. The mixture cooled to room temperature, poured into water (25 mL) and extracted with EtOAc (3×50 mL). EtOAc extracts combined, washed with saturated aqueous NaCl (25 mL), dried over Na₂SO₄, and concentrated. Product purified by silica gel chromatography (eluent: 85-100% EtOAc-hexanes gradient). Yield: 60%. MS (m/z): 328.2 (MH⁺).

### Preparation of (Z)-1-(2-((5-methoxy-2-(1-(2-methoxyethyl)-3,5-dimethyl-1H-pyrazol-4-yl)-1H-indol-3-yl)methylene)-3-oxo-2,3-dihydrobenzofuran-5-yl)-3-methylurea

Concentrated aqueous HCl (5 drops) was added to a stirred mixture of 5-methoxy-2-(1-(2-methoxyethyl)-3,5-dimethyl-1H-pyrazol-4-yl)-1H-indole-3-carbaldehyde (70 mg, 0.21 mmol) and 1-methyl-3-(3-oxo-2,3-dihydrobenzofuran-5-yl)urea (53 mg, 0.26 mmol) in EtOH (1.5 mL). Resulting mixture stirred overnight at 55° C. The mixture cooled to room temperature, diluted with additional EtOH (5 mL) then added to saturated aqueous Na₂CO₃ (5 mL) and then stirred at 65° C. for 20 minutes. Deep red organic layer was collected and concentrated. Residue dissolved in MeOH, filtered, and subjected to preparative HPLC. Yield: 35%. MS (m/z): 516.2 (MH⁺).

### (Z)-1-(2-((2-(1-(2-(Dimethylamino)ethyl)-3,5-dimethyl-1H-pyrazol-4-yl)-5-methoxy-1H-indol-3-yl)methylene)-3-oxo-2,3-dihydrobenzofuran-5-yl)-3-methyl urea MS (m/z): 529.3 (M H⁺)

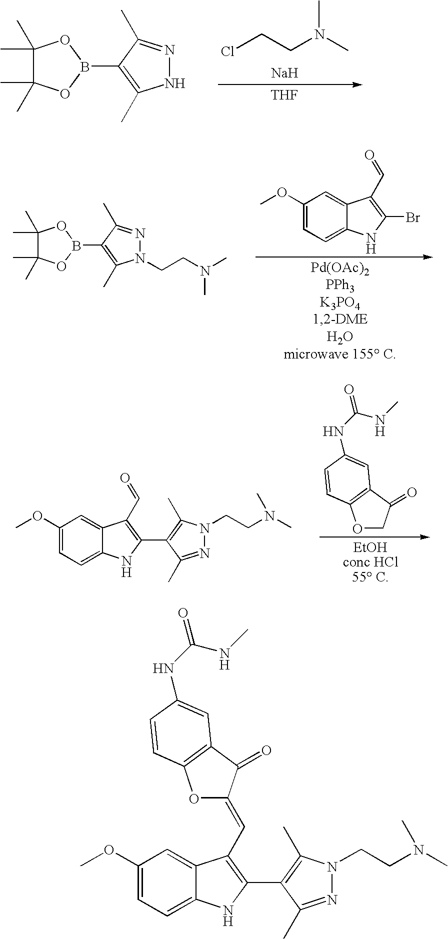

### Preparation of 2-(3,5-dimethyl-4-(4,4,5,5-tetramethyl-1,3,2-dioxaborolan-2-yl)-1H-pyrazol-1-yl)-N,N-dimethylethanamine

Sodium hydride (40 mg, 1.69 mmol) was added to a stirred solution of 3,5-dimethyl-4-(4,4,5,5-tetramethyl-1,3,2-dioxaborolan-2-yl)-1H-pyrazole (313 mg, 1.41 mmol) in THF (9.40 mL). Resulting mixture stirred for 5 minutes then 2-chloro-N,N-dimethylethanamine (182 mg, 1.69 mmol) added. (2-Chloro-N,N-dimethylethanamine was prepared from its corresponding HCl salt by partitioning between 20% Et₂O-Hex and 5 M aqueous NaOH, drying the organic layer over Na₂SO₄, removal of solvent, and then using the resulting residue directly). Resulting mixture stirred for 30 minutes at room temperature, then stirred overnight at 60° C. The mixture poured into 1:1 H₂O-saturated aqueous NaCl (25 mL) and extracted with EtOAc (2×50 mL). EtOAc layers combined, washed with saturated aqueous NaCl (25 mL), dried over Na₂SO₄, and concentrated to give an oil. The oil was triturated with hexanes (15 mL) and filtered. Filtrate collected and concentrated in vacuo. Product used immediately.

### Preparation of 2-(1-(2-(dimethylamino)ethyl)-3,5-dimethyl-1H-pyrazol-4-yl)-5-methoxy-1H-indole-3-carbaldehyde

A mixture of 2-bromo-5-methoxy-1H-indole-3-carbaldehyde (240 mg, 0.94 mmol), 2-(3,5-dimethyl-4-(4,4,5,5-tetramethyl-1,3,2-dioxaborolan-2-yl)-1H-pyrazol-1-yl)-N,N-dimethylethanamine (336 mg, 1.15 mmol), diacetoxy palladium (8 mg, 0.04 mmol), triphenylphosphine (30 mg, 0.11 mmol), and potassium phosphate (602 mg, 2.83 mmol) in DME (2.2 mL) and water (1.4 mL) was subjected to microwave conditions (155° C., 30 min). The mixture cooled to room temperature, poured into 1 M aqueous HCl (25 mL) and EtOAc (50 mL). Organic layer extracted with 1 M aqueous HCl (25 mL). Aqueous layers combined and extracted with EtOAc (2×25 mL), then basified to pH˜8-9 using saturated aqueous Na₂CO₃. Basified aqueous layer extracted with EtOAc (3×50 mL). EtOAc extracts of the basic aqueous layer were combined, washed with saturated aqueous NaCl (25 mL), dried over Na₂SO₄, and concentrated. Yield: 77%. MS (m/z): 341.4 (MH⁺).

### Preparation of (Z)-1-(2-((2-(1-(2-(dimethylamino)ethyl)-3,5-dimethyl-1H-pyrazol-4-yl)-5-methoxy-1H-indol-3-yl)methylene)-3-oxo-2,3-dihydrobenzofuran-5-yl)-3-methylurea

Concentrated aqueous HCl (6 drops) was added to a stirred mixture of 2-(1-(2-(dimethylamino)ethyl)-3,5-dimethyl-1H-pyrazol-4-yl)-5-methoxy-1H-indole-3-carbaldehyde (100 mg, 0.29 mmol) and 1-methyl-3-(3-oxo-2,3-dihydrobenzofuran-5-yl)urea (73 mg, 0.35 mmol) in EtOH (2 mL). Resulting mixture stirred at 55° C. for 3 hours, then at room temperature overnight. EtOAc (3 mL) added and mixture suction filtered through sintered glass. Filtrate collected and concentrated. Crude product purified by preparative HPLC. Yield: 52%. MS (m/z): 529.3 (MH⁺)

### (Z)-1-(2-((1-(3-Cyanopropyl)-5-methoxy-2-(1,3,5-trimethyl-1H-pyrazol-4-yl)-1H-indol-3-yl)methylene)-3-oxo-2,3-dihydrobenzofuran-5-yl)-3-methyl urea MS (m/z): 539.2 (M H⁺)

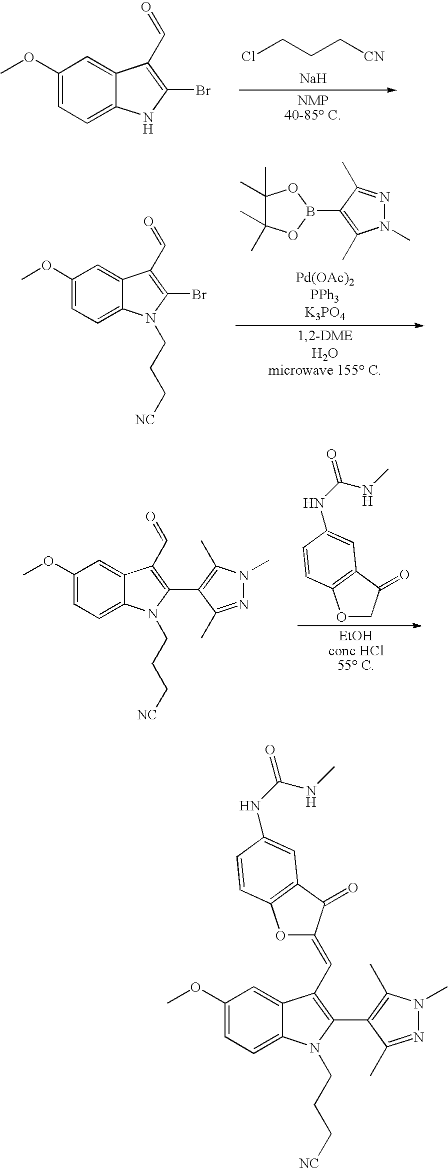

### Preparation of 4-(2-bromo-3-formyl-5-methoxy-1H-indol-1-yl)butanenitrile

A solution of 2-bromo-5-methoxy-1H-indole-3-carbaldehyde (100 mg, 0.39 mmol) in NMP (1.2 mL) added slowly to NaH (excess) at room temperature. Resulting mixture stirred for 25 minutes then 4-chlorobutyronitrile (46 μL, 0.51 mmol) added. Reaction mixture heated to 40° C. and stirred for 90 minutes, then stirred overnight at 85° C. The mixture cooled to room temperature, poured into saturated aqueous NaCl (25 mL) and extracted with EtOAc (2×50 mL). EtOAc extracts combined, washed with saturated aqueous NaCl (2×25 mL), dried over Na₂SO₄ and concentrated. The mixture purified by silica gel column chromatography (eluent: 20-35% EtOAc-hexanes gradient). Yield 88%. MS (m/z): 321.0 (MH⁺).

### Preparation of 4-(3-formyl-5-methoxy-2-(1,3,5-trimethyl-1H-pyrazol-4-yl)-1H-indol-1-yl)butanenitrile

A mixture of 4-(2-bromo-3-formyl-5-methoxy-1H-indol-1-yl)butanenitrile (102 mg, 0.32 mmol), 1,3,5-trimethyl-1-H-pyrazole-4-boronic acid pinacol ester (106 mg, 0.45 mmol), Pd(OAc)₂ (3 mg, 0.01 mmol), PPh₃ (10 mg, 0.04 mmol), K₃PO₄ (204 mg, 0.96 mmol) in 1,2-dimethoxyethane (1.5 mL) and water (1 mL) was subjected to microwave conditions (155° C., 40 min). Reaction mixture cooled to room temperature, poured into water (25 mL) and extracted with EtOAc (2×50 mL). EtOAc extracts combined, washed with saturated aqueous NaCl (25 mL), dried over Na₂SO₄, and concentrated. The mixture purified by silica gel column chromatography (eluent: 75-100% EtOAc-hexanes). Yield 68%. MS (m/z): 351.2 (MH⁺).

### Preparation of (Z)-1-(2-((1-(3-cyanopropyl)-5-methoxy-2-(1,3,5-trimethyl-1H-pyrazol-4-yl)-1H-indol-3-yl)methylene)-3-oxo-2,3-dihydrobenzofuran-5-yl)-3-methylurea

Concentrated aqueous HCl (3 drops) was added to a stirred mixture of 4-(3-formyl-5-methoxy-2-(1,3,5-trimethyl-1H-pyrazol-4-yl)-1H-indol-1-yl)butanenitrile (70 mg, 0.20 mmol) and 1-methyl-3-(3-oxo-2,3-dihydrobenzofuran-5-yl)urea (49 mg, 0.24 mmol) in EtOH (1.5 mL). Resulting mixture stirred overnight at 40° C. and then 55° C. for 5 hours. Reaction mixture stored at 5° C. for 1 week. EtOAc (2 mL) added and mixture filtered. Filtrate treated with K₂CO₃ (300 mg) and diluted with EtOH (5 mL) and water (0.5 mL). Resulting mixture stirred at 70° C. for 15 minutes then cooled to room temperature. Organic layer collected and concentrated. Residue purified by preparative HPLC. Yield 24%. MS (m/z): 539.2 (MH⁺).

### (Z)-1-(2-((5-Methoxy-2-(4-methylpiperazin-1-yl)-1-(2-(4-methylpiperazin-1-yl)ethyl)-1H-indol-3-yl)methylene)-3-oxo-2,3-dihydrobenzofuran-5-yl)-3-methyl urea MS (m/z): 588.3 (M H⁺)

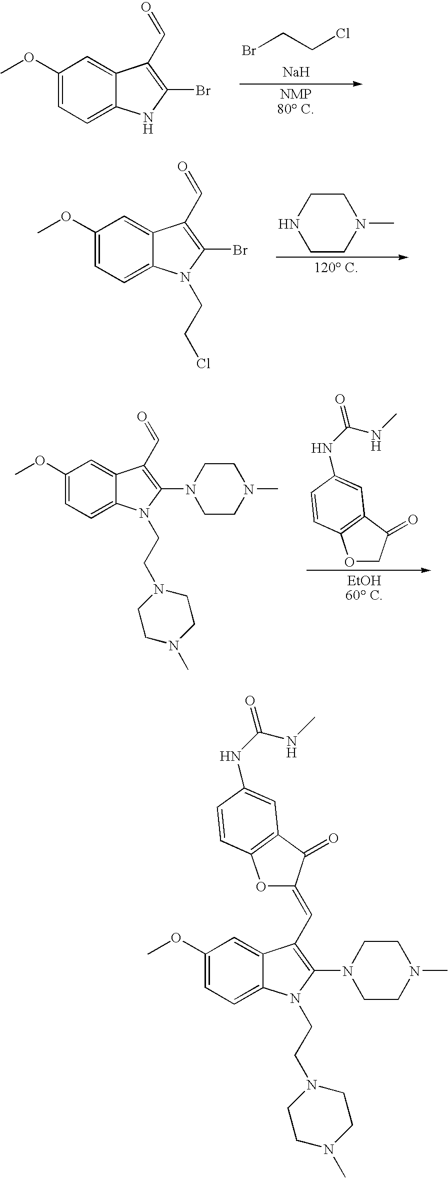

### Preparation of 2-bromo-1-(2-chloroethyl)-5-methoxy-1H-indole-3-carbaldehyde

A solution of 2-bromo-5-methoxy-1H-indole-3-carbaldehyde (300 mg, 1.18 mmol) in NMP (2 mL) was added to NaH (40 mg, 1.67 mmol) over a period of 1 minutes. Resulting mixture stirred at room temperature for 30 minutes, then at 80° C. for 10 minutes. 1-Bromo-2-chloroethane (490 μL, 5.9 mmol) was added and reaction mixture stirred at 80° C. for 5 hours. Reaction mixture cooled to room temperature, poured into saturated aqueous NaCl (25 mL) and extracted with EtOAc (100 mL). EtOAc layer washed with saturated aqueous NaCl (3×25 mL), dried over Na₂SO₄ and concentrated. Yield 100%. MS (m/z): 316.0 (MH⁺).

### Preparation of 5-methoxy-2-(4-methylpiperazin-1-yl)-1-(2-(4-methylpiperazin-1-yl)ethyl)-1H-indole-3-carbaldehyde

A solution of 2-bromo-1-(2-chloroethyl)-5-methoxy-1H-indole-3-carbaldehyde (365 mg, 1.15 mmol) in 1-methylpiperazine (3 mL) was heated to 105° C. for 2.5 hours, then 120° C. for 2 hours. Reaction mixture cooled to room temperature, poured into 1:1 saturated aqueous NaCl—H₂O (40 mL) and extracted with EtOAc (2×50 mL). EtOAc layers combined, dried over Na₂SO₄ and concentrated.

### Preparation of (Z)-1-(2-((5-methoxy-2-(4-methylpiperazin-1-yl)-1-(2-(4-methylpiperazin-1-yl)ethyl)-1H-indol-3-yl)methylene)-3-oxo-2,3-dihydrobenzofuran-5-yl)-3-methylurea

A mixture of 5-methoxy-2-(4-methylpiperazin-1-yl)-1-(2-(4-methylpiperazin-1-yl)ethyl)-1H-indole-3-carbaldehyde (95 mg, 0.24 mmol) and 1-methyl-3-(3-oxo-2,3-dihydrobenzofuran-5-yl)urea (52 mg, 0.25 mmol) in EtOH (1.5 mL) was stirred at 60° C. for 2 days. Reaction mixture cooled to room temperature and purified directly by silica gel column chromatography (eluent: 70:20:10 CH₃CN-Et₃N-MeOH). Yield 47%. MS (m/z): 588.3 (MH⁺).

### (Z)-1-(2-((5-Methoxy-1-(2-(4-methylpiperazin-1-yl)ethyl)-2-(1,3,5-trimethyl-1H-pyrazol-4-yl)-1H-indol-3-yl)methylene)-3-oxo-2,3-dihydrobenzofuran-5-yl)-3-methyl urea MS (m/z): 598.3 (M H⁺)

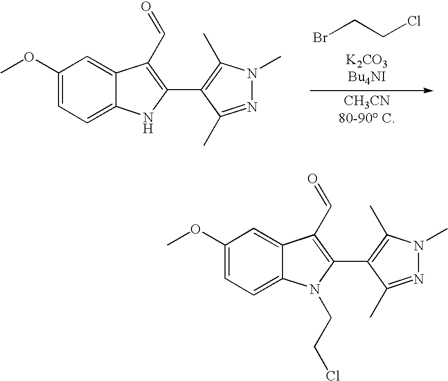

### Preparation of 1-(2-chloroethyl)-5-methoxy-2-(1,3,5-trimethyl-1H-pyrazol-4-yl)-1H-indole-3-carbaldehyde

A mixture of 5-methoxy-2-(1,3,5-trimethyl-1H-pyrazol-4-yl)-1H-indole-3-carbaldehyde (327 mg, 1.15 mmol), 1-bromo-2-chloroethane (765 μL, 9.23 mmol), K₂CO₃ (1.12 g, 8.1 mmol), and Bu₄NI (40 mg) in CH₃CN (5.8 mL) was stirred at 80° C. overnight. The mixture cooled to room temperature, poured into water (25 mL) and extracted with EtOAc (3×50 mL). EtOAc extracts combined, washed with saturated aqueous NaCl (25 mL), dried over Na₂SO₄, and concentrated. The mixture purified by silica gel column chromatography (eluent: 80-100% EtOAc-hexanes gradient). Yield 93%.

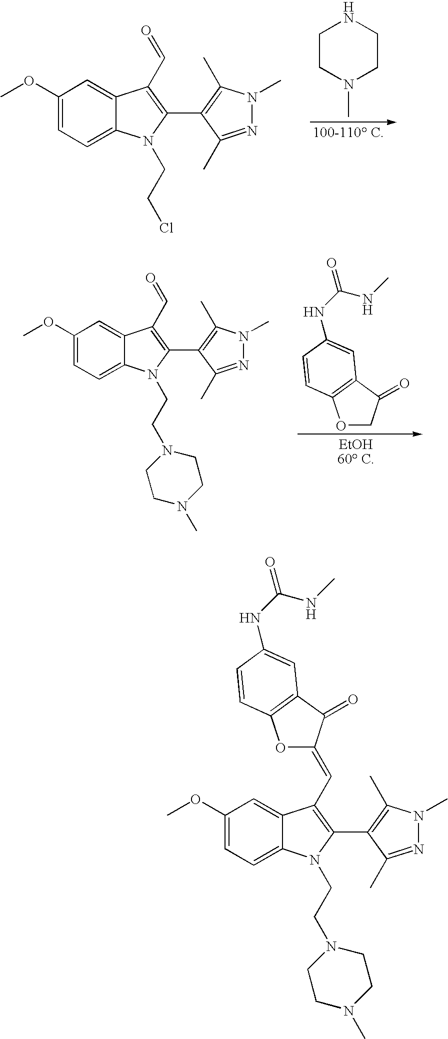

### Preparation of 5-methoxy-1-(2-(4-methylpiperazin-1-yl)ethyl)-2-(1,3,5-trimethyl-1H-pyrazol-4-yl)-1H-indole-3-carbaldehyde

A mixture of 1-(2-chloroethyl)-5-methoxy-2-(1,3,5-trimethyl-1H-pyrazol-4-yl)-1H-indole-3-carbaldehyde (90 mg) and 1-methylpiperazine (2.5 mL) was heated between 100-110° C. over a total period of 12 hours. Reaction mixture cooled to room temperature and concentrated in vacuo. Crude product partitioned between EtOAc and 0.5 M aqueous HCl. Aqueous layer extracted twice with EtOAc. Aqueous layer made basic (pH 9) using saturated aqueous Na₂CO₃, then extracted with EtOAc (3×). EtOAc extracts of basic aqueous layer combined, washed with saturated aqueous NaCl, dried over Na₂SO₄, and concentrated. Yield 40%. MS (m/z): 410.2 (MH⁺).

### Preparation of (Z)-1-(2-((5-methoxy-1-(2-(4-methylpiperazin-1-yl)ethyl)-2-(1,3,5-trimethyl-1H-pyrazol-4-yl)-1H-indol-3-yl)methylene)-3-oxo-2,3-dihydrobenzofuran-5-yl)-3-methylurea

Concentrated aqueous HCl (6 drops) was added to a stirred mixture of 5-methoxy-1-(2-(4-methylpiperazin-1-yl)ethyl)-2-(1,3,5-trimethyl-1H-pyrazol-4-yl)-1H-indole-3-carbaldehyde (48 mg, 0.12 mmol) and 1-methyl-3-(3-oxo-2,3-dihydrobenzofuran-5-yl)urea (29 mg, 0.14 mmol) in EtOH (1.0 mL). Resulting mixture stirred at 60° C. for 3 hours. The mixture cooled to room temperature, diluted with additional EtOH (5 mL) then added to saturated aqueous Na₂CO₃ (3 mL) then stirred at 65° C. for 25 minutes. The mixture cooled to room temperature, diluted with EtOH (10 mL), filtered, and filtrate collected and concentrated. Residue purified by preparative HPLC. Yield: 31%. MS (m/z): 598.3 (MH⁺).

### (Z)-1-(2-((1-(2-(2-Hydroxyethylamino)ethyl)-5-methoxy-2-(1,3,5-trimethyl-1H-pyrazol-4-yl)-1H-indol-3-yl)methylene)-3-oxo-2,3-dihydrobenzofuran-5-yl)-3-methyl urea MS (m/z): 559.3 (M H⁺)

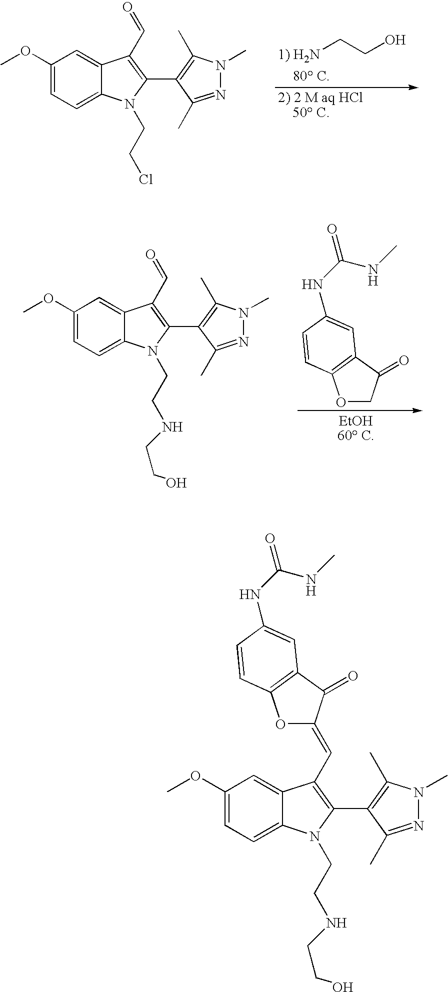

### Preparation of 1-(2-(2-hydroxyethylamino)ethyl)-5-methoxy-2-(1,3,5-trimethyl-1H-pyrazol-4-yl)-1H-indole-3-carbaldehyde

A mixture of 1-(2-chloroethyl)-5-methoxy-2-(1,3,5-trimethyl-1H-pyrazol-4-yl)-1H-indole-3-carbaldehyde (112 mg) and ethanolamine (2 mL) was heated to 80° C. overnight. Reaction mixture cooled to room temperature and 2 M aqueous HCl (30 mL) added and resulting mixture stirred at 50° C. for 90 minutes. The mixture cooled to room temperature and made basic (pH 8-9) using saturated aqueous Na₂CO₃ and extracted with EtOAc (3×40 mL). EtOAc extracts combined, washed with 1:1 saturated aqueous NaCl-water (2×15 mL), then saturated aqueous NaCl (25 mL), dried over Na₂SO₄ and concentrated. MS (m/z): 371.2 (MH⁺).

### Preparation of (Z)-1-(2-((1-(2-(2-hydroxyethylamino)ethyl)-5-methoxy-2-(1,3,5-trimethyl-1H-pyrazol-4-yl)-1H-indol-3-yl)methylene)-3-oxo-2,3-dihydrobenzofuran-5-yl)-3-methylurea

Concentrated aqueous HCl (5 drops) was added to a stirred mixture of 1-(2-(2-hydroxyethylamino)ethyl)-5-methoxy-2-(1,3,5-trimethyl-1H-pyrazol-4-yl)-1H-indole-3-carbaldehyde (80 mg, 0.22 mmol) and 1-methyl-3-(3-oxo-2,3-dihydrobenzofuran-5-yl)urea (54 mg, 0.26 mmol) in EtOH (1.0 mL). Resulting mixture stirred at 60° C. for 2 hours. The mixture cooled to room temperature, diluted with additional EtOH (5 mL), and then neutralized using saturated aqueous Na₂CO₃. The mixture filtered, and filtrate collected and concentrated. Residue purified by preparative HPLC. Yield: 9%. MS (m/z): 559.3 (MH⁺).

### (Z)-1-(2-((1-(2-((2-(Dimethylamino)ethyl)(methyl)amino)ethyl)-5-methoxy-2-(1,3,5-trimethyl-1H-pyrazol-4-yl)-1H-indol-3-yl)methylene)-3-oxo-2,3-dihydrobenzofuran-5-yl)-3-methyl urea MS (m/z): 600.3 (MH⁺)

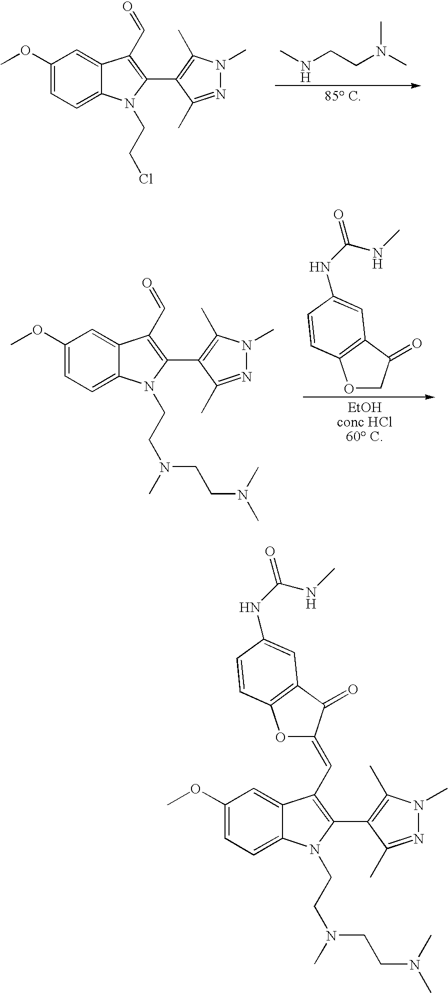

### Preparation of 1-(2-((2-(dimethylamino)ethyl)(methyl)amino)ethyl)-5-methoxy-2-(1,3,5-trimethyl-1H-pyrazol-4-yl)-1H-indole-3-carbaldehyde

A mixture of 1-(2-chloroethyl)-5-methoxy-2-(1,3,5-trimethyl-1H-pyrazol-4-yl)-1H-indole-3-carbaldehyde (112 mg, 0.32 mmol) and N,N,N′-trimethylethylenediamine (2 mL) was heated to 85° C. overnight. The mixture cooled to room temperature and treated with 1 M aqueous HCl (25 mL), diluted with water (10 mL), and extracted with EtOAc (2×40 mL). Aqueous layer made basic (pH 8-9) using saturated aqueous Na₂CO₃ and extracted with EtOAc (3×40 mL). EtOAc extracts of the basic aqueous layer combined, washed with saturated aqueous NaCl (25 mL), dried over Na₂SO₄, and concentrated. Yield: 61%. MS (m/z): 412.3 (MH⁺).

### Preparation of (Z)-1-(2-((1-(2-((2-(dimethylamino)ethyl)(methyl)amino)ethyl)-5-methoxy-2-(1,3,5-trimethyl-1H-pyrazol-4-yl)-1H-indol-3-yl)methylene)-3-oxo-2,3-dihydrobenzofuran-5-yl)-3-methylurea

Concentrated aqueous HCl (5 drops) was added to a stirred mixture of 1-(2-((2-(dimethylamino)ethyl)(methyl)amino)ethyl)-5-methoxy-2-(1,3,5-trimethyl-1H-pyrazol-4-yl)-1H-indole-3-carbaldehyde (78 mg, 0.19 mmol) and 1-methyl-3-(3-oxo-2,3-dihydrobenzofuran-5-yl)urea (47 mg, 0.23 mmol) in EtOH (1.2 mL). Resulting mixture stirred overnight at 50° C. Additional 1-methyl-3-(3-oxo-2,3-dihydrobenzofuran-5-yl)urea (25 mg, 0.12 mmol) added and mixture stirred at 60° C. for 4 hours. The mixture cooled to room temperature and made basic (pH˜9) using saturated aqueous Na₂CO₃. Resulting mixture stirred at 65° C. for 30 minutes, cooled to room temperature, diluted with EtOH (10 mL), poured into EtOAc (50 mL), and then filtered. Filtrate collected and concentrated. Residue purified by preparative HPLC. Yield: 13%. MS (m/z): 600.3 (MH⁺).

### (Z)-1-(2-((1-(2-(2-(Dimethylamino)ethylamino)ethyl)-5-methoxy-2-(1,3,5-trimethyl-1H-pyrazol-4-yl)-1H-indol-3-yl)methylene)-3-oxo-2,3-dihydrobenzofuran-5-yl)-3-methyl urea MS (m/z): 586.3 (MH⁺)

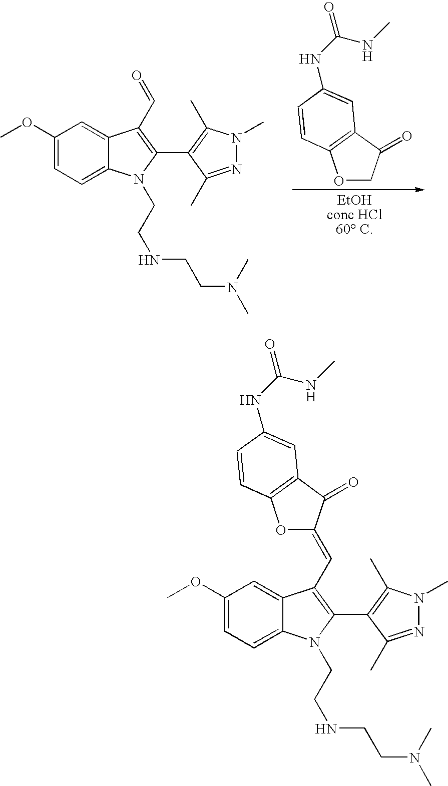

### Preparation of 1-(2-(2-(dimethylamino)ethylamino)ethyl)-5-methoxy-2-(1,3,5-trimethyl-1H-pyrazol-4-yl)-1H-indole-3-carbaldehyde

Method as described for the preparation of 1-(2-((2-(dimethylamino)ethyl)(methyl)amino)ethyl)-5-methoxy-2-(1,3,5-trimethyl-1H-pyrazol-4-yl)-1H-indole-3-carbaldehyde except using N,N-dimethylethylenediamine as the amine. Yield: 89%. MS (m/z): 398.3 (MH⁺).

### Preparation of (Z)-1-(2-((1-(2-(2-(dimethylamino)ethylamino)ethyl)-5-methoxy-2-(1,3,5-trimethyl-1H-pyrazol-4-yl)-1H-indol-3-yl)methylene)-3-oxo-2,3-dihydrobenzofuran-5-yl)-3-methylurea

Concentrated aqueous HCl (7 drops) was added to a stirred mixture of 1-(2-(2-(dimethylamino)ethylamino)ethyl)-5-methoxy-2-(1,3,5-trimethyl-1H-pyrazol-4-yl)-1H-indole-3-carbaldehyde (112 mg, 0.28 mmol) and 1-methyl-3-(3-oxo-2,3-dihydrobenzofuran-5-yl)urea (70 mg, 0.34 mmol) in EtOH (2 mL). Resulting mixture stirred overnight at 50° C. and then at 60° C. for 4 hours. The mixture cooled to room temperature and made basic (pH˜9) using saturated aqueous Na₂CO₃. Resulting mixture stirred at 65° C. for 30 minutes, cooled to room temperature, diluted with EtOH (10 mL), poured into EtOAc (50 mL), and then filtered. Filtrate collected and concentrated. Residue purified by preparative HPLC. Yield: 20%. MS (m/z): 586.3 (MH⁺).

### (Z)-1-(2-((5-Methoxy-1-(2-(piperazin-1-yl)ethyl)-2-(1,3,5-trimethyl-1H-pyrazol-4-yl)-1H-indol-3-yl)methylene)-3-oxo-2,3-dihydrobenzofuran-5-yl)-3-methyl urea MS (m/z): 584.3 (M H⁺)

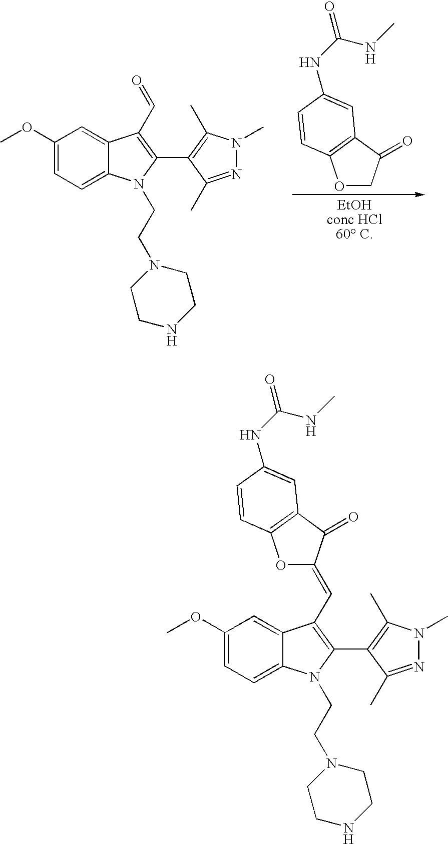

### Preparation of 5-methoxy-1-(2-(piperazin-1-yl)ethyl)-2-(1,3,5-trimethyl-1H-pyrazol-4-yl)-1H-indole-3-carbaldehyde

Method as described for the preparation of 1-(2-((2-(dimethylamino)ethyl)(methyl)amino)ethyl)-5-methoxy-2-(1,3,5-trimethyl-1H-pyrazol-4-yl)-1H-indole-3-carbaldehyde except using piperazine as the amine. Yield: 83%. MS (m/z): 396.3 (MH⁺).

### Preparation of (Z)-1-(2-((5-methoxy-1-(2-(piperazin-1-yl)ethyl)-2-(1,3,5-trimethyl-1H-pyrazol-4-yl)-1H-indol-3-yl)methylene)-3-oxo-2,3-dihydrobenzofuran-5-yl)-3-methylurea

Concentrated aqueous HCl (7 drops) was added to a stirred mixture of 5-methoxy-1-(2-(piperazin-1-yl)ethyl)-2-(1,3,5-trimethyl-1H-pyrazol-4-yl)-1H-indole-3-carbaldehyde (127 mg, 0.32 mmol) and 1-methyl-3-(3-oxo-2,3-dihydrobenzofuran-5-yl)urea (78 mg, 0.38 mmol) in EtOH (2.4 mL). Resulting mixture stirred overnight at 50° C. and then at 60° C. for 4 hours. The mixture cooled to room temperature and made basic (pH˜9) using saturated aqueous Na₂CO₃. Resulting mixture stirred at 65° C. for 30 minutes, cooled to room temperature, diluted with EtOH (10 mL), poured into EtOAc (50 mL), and then filtered. Filtrate collected and concentrated. Residue purified by preparative HPLC. Yield: 14%. MS (m/z): 584.3 (MH⁺).

### (Z)-1-(2-((5-Methoxy-1-(2-(methylamino)ethyl)-2-(1,3,5-trimethyl-1H-pyrazol-4-yl)-1H-indol-3-yl)methylene)-3-oxo-2,3-dihydrobenzofuran-5-yl)-3-methyl urea MS (m/z): 529.3 (M H⁺)

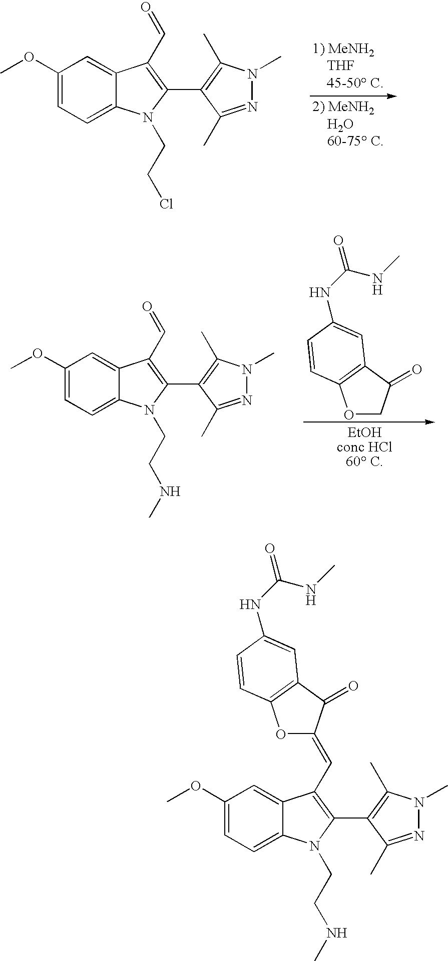

### Preparation of 5-methoxy-1-(2-(methylamino)ethyl)-2-(1,3,5-trimethyl-1H-pyrazol-4-yl)-1H-indole-3-carbaldehyde

A solution of 1-(2-chloroethyl)-5-methoxy-2-(1,3,5-trimethyl-1H-pyrazol-4-yl)-1H-indole-3-carbaldehyde (140 mg, 0.40 mmol) and methylamine (2.0 M in THF, 3 mL) was heated to 45° C. for 5 days, and then 50° C. for 5 days in a sealed tube. Solvent removed and residue treated with 40% aqueous methylamine (3 mL) and resulting mixture stirred in a sealed pressure tube at 60° C. for 3 days and 75° C. for 1 day. The mixture cooled to room temperature and treated with water (5 mL) and 6 M aqueous HCl until pH˜2 and stirred for 90 minutes. The mixture extracted with EtOAc (3×30 mL). Aqueous layer made basic (pH˜9) using saturated aqueous Na₂CO₃ and extracted with EtOAc (3×50 mL). EtOAc extracts of the basic aqueous layer combined, washed with saturated aqueous NaCl (25 mL), dried over Na₂SO₄, and concentrated. Yield: 85%. MS (m/z): 341.2 (MH⁺).

### Preparation of (Z)-1-(2-((5-methoxy-1-(2-(methylamino)ethyl)-2-(1,3,5-trimethyl-1H-pyrazol-4-yl)-1H-indol-3-yl)methylene)-3-oxo-2,3-dihydrobenzofuran-5-yl)-3-methylurea

Concentrated aqueous HCl (6 drops) was added to a stirred mixture of 5-methoxy-1-(2-(methylamino)ethyl)-2-(1,3,5-trimethyl-1H-pyrazol-4-yl)-1H-indole-3-carbaldehyde (104 mg, 0.31 mmol) and 1-methyl-3-(3-oxo-2,3-dihydrobenzofuran-5-yl)urea (68 mg, 0.33 mmol) in EtOH (1.6 mL). Resulting mixture stirred at 60° C. for 2 hours. Additional 1-methyl-3-(3-oxo-2,3-dihydrobenzofuran-5-yl)urea (35 mg, 0.17 mmol) added and mixture stirred at 60° C. for 90 minutes and then overnight at 40° C. The mixture cooled to room temperature, poured into water (50 mL), stirred for 30 minutes, and then filtered through Celite™. Filtrate made basic using saturated aqueous Na₂CO₃ and then concentrated. Resulting residue taken up in EtOH and then filtered. Filtrate concentrated and residue purified by preparative HPLC. Yield: 27%. MS (m/z): 529.3 (MH⁺).

### (Z)-1-(2-((1-(2-(Dimethylamino)ethyl)-5-methoxy-2-(1,3,5-trimethyl-1H-pyrazol-4-yl)-1H-indol-3-yl)methylene)-3-oxo-2,3-dihydrobenzofuran-5-yl)-3-methylurea MS (m/z): 543.3 (MH⁺)

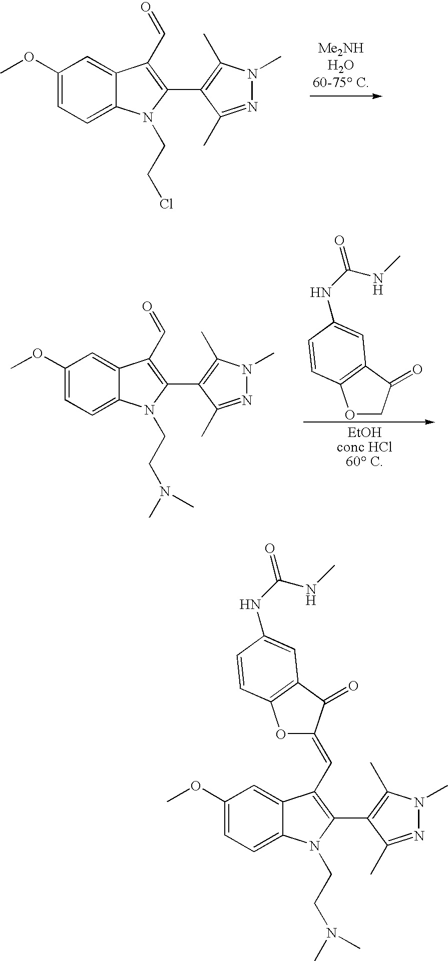

### Preparation of 1-(2-(dimethylamino)ethyl)-5-methoxy-2-(1,3,5-trimethyl-1H-pyrazol-4-yl)-1H-indole-3-carbaldehyde

A mixture of 1-(2-chloroethyl)-5-methoxy-2-(1,3,5-trimethyl-1H-pyrazol-4-yl)-1H-indole-3-carbaldehyde (176 mg, 0.51 mmol) and dimethylamine (40% in water, 2.5 mL) was stirred in a sealed pressure tube at 65° C. overnight, then 75° C. for 6 hours. The mixture cooled to room temperature and excess dimethylamine removed using a stream of N₂. The mixture acidified to pH˜2 with 3 M aqueous HCl and diluted with water (25 mL) and extracted with EtOAc (2×25 mL). Aqueous layer made basic (pH 8-9) using saturated aqueous Na₂CO₃ and extracted with EtOAc (3×40 mL). EtOAc extracts of the basic aqueous layer combined, washed with saturated aqueous NaCl (25 mL), dried over Na₂SO₄, and concentrated. Yield: 70%. MS (m/z): 355.2 (MH⁺).

### Preparation of (Z)-1-(2-((1-(2-(dimethylamino)ethyl)-5-methoxy-2-(1,3,5-trimethyl-1H-pyrazol-4-yl)-1H-indol-3-yl)methylene)-3-oxo-2,3-dihydrobenzofuran-5-yl)-3-methylurea

Concentrated aqueous HCl (6 drops) was added to a stirred mixture of 1-(2-(dimethylamino)ethyl)-5-methoxy-2-(1,3,5-trimethyl-1H-pyrazol-4-yl)-1H-indole-3-carbaldehyde (60 mg, 0.17 mmol) and 1-methyl-3-(3-oxo-2,3-dihydrobenzofuran-5-yl)urea (41 mg, 0.20 mmol) in EtOH (1.5 mL). Resulting mixture stirred at 60° C. for 4 hours. The mixture cooled to room temperature and neutralized using saturated aqueous Na₂CO₃. The mixture sat overnight at room temperature and then filtered. Filtrate concentrated and residue dissolved in MeOH and the mixture filtered. Filtrate concentrated and then purified by preparative HPLC. Yield: 32%. MS (m/z): 543.3 (MH⁺).

### (Z)-1-(2-((5-(2-Methoxyethoxy)-2-(1,3,5-trimethyl-1H-pyrazol-4-yl)-1H-indol-3-yl)methylene)-3-oxo-2,3-dihydrobenzofuran-5-yl)-3-methylurea MS (m/z): 516.2 (MH⁺)

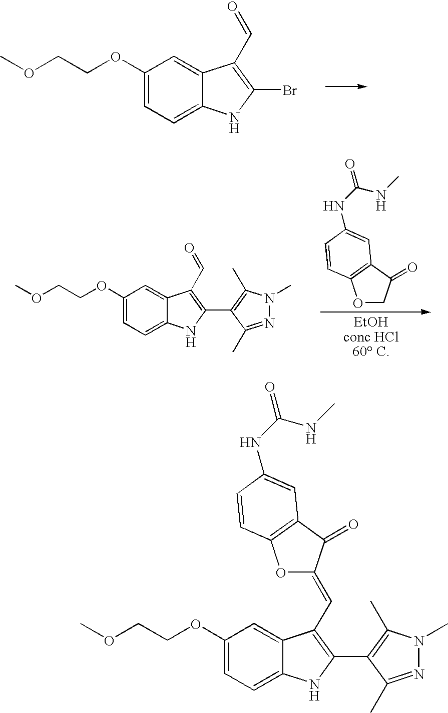

### Preparation of 2-bromo-5-(2-methoxyethoxy)-1H-indole-3-carbaldehyde

Prepared via Gasman oxindole-Vilsmeier-Haack reactions using 4-(2-methoxyethoxy)aniline. Purified by silica gel chromatography (eluent: 50% EtOAc-hexanes to 50% EtOAc-CH₂Cl₂ gradient). MS (m/z): 296.1 (MH−).

### Preparation of 5-(2-methoxyethoxy)-2-(1,3,5-trimethyl-1H-pyrazol-4-yl)-1H-indole-3-carbaldehyde

Preparation via the Suzuki coupling method using 2-bromo-5-(2-methoxyethoxy)-1H-indole-3-carbaldehyde. Purified by silica gel chromatography (eluent: 0-5% MeOH-EtOAc gradient). Yield 48%. MS (m/z): 328.2 (MH⁺).

### Preparation of (Z)-1-(2-((5-(2-methoxyethoxy)-2-(1,3,5-trimethyl-1H-pyrazol-4-yl)-1H-indol-3-yl)methylene)-3-oxo-2,3-dihydrobenzofuran-5-yl)-3-methylurea

Concentrated aqueous HCl (6 drops) was added to a stirred mixture of 5-(2-methoxyethoxy)-2-(1,3,5-trimethyl-1H-pyrazol-4-yl)-1H-indole-3-carbaldehyde (89 mg, 0.27 mmol) and 1-methyl-3-(3-oxo-2,3-dihydrobenzofuran-5-yl)urea (66 mg, 0.32 mmol) in EtOH (1.5 mL). Resulting mixture stirred at 60° C. for 4 hours. The mixture cooled to room temperature, diluted with EtOAc (3 mL) and filtered. Filtrate treated with saturated aqueous Na₂CO₃ (5 mL), stirred for 5 minutes, and then decanted into EtOH (50 mL). The mixture filtered and concentrated and residue purified by preparative HPLC. Yield: 35%. MS (m/z): 516.2 (MH⁺).

### (Z)-1-(2-((6-Fluoro-5,7-dimethoxy-2-(1,3,5-trimethyl-1H-pyrazol-4-yl)-1H-indol-3-yl)methylene)-3-oxo-2,3-dihydrobenzofuran-5-yl)-3-methylurea MS (m/z): 520.2 (MH⁺)

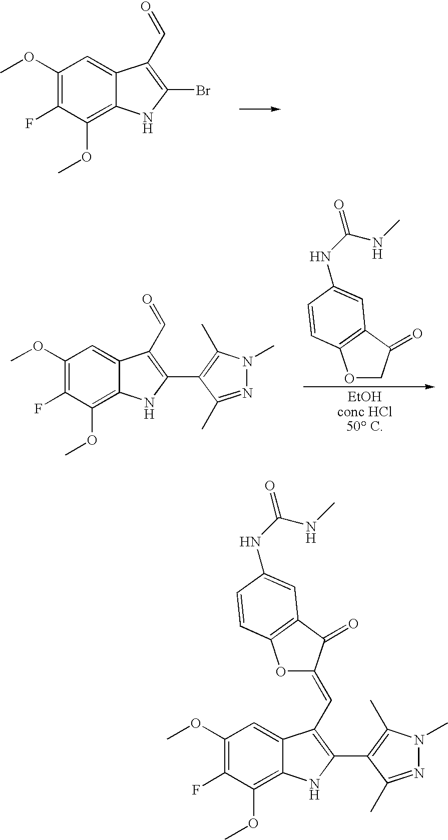

### Preparation of 2-bromo-6-fluoro-5,7-dimethoxy-1H-indole-3-carbaldehyde

Prepared via Gassman oxindole-Vilsmeier-Haack reactions using 3-fluoro-2,4-dimethoxyaniline. MS (m/z): 302.0 (MH⁺).

### Preparation of 6-fluoro-5,7-dimethoxy-2-(1,3,5-trimethyl-1H-pyrazol-4-yl)-1H-indole-3-carbaldehyde

Preparation via the Suzuki coupling method using 2-bromo-6-fluoro-5,7-dimethoxy-1H-indole-3-carbaldehyde. MS (m/z): 332.1 (MH⁺).

### Preparation of (Z)-1-(2-((6-fluoro-5,7-dimethoxy-2-(1,3,5-trimethyl-1H-pyrazol-4-yl)-1H-indol-3-yl)methylene)-3-oxo-2,3-dihydrobenzofuran-5-yl)-3-methylurea

Concentrated aqueous HCl (3 drops) was added to a stirred mixture of 6-fluoro-5,7-dimethoxy-2-(1,3,5-trimethyl-1H-pyrazol-4-yl)-1H-indole-3-carbaldehyde (116 mg, 0.35 mmol) and 1-methyl-3-(3-oxo-2,3-dihydrobenzofuran-5-yl)urea (87 mg, 0.42 mmol) in EtOH (1.5 mL). The mixture stirred at 50° C. for 2 hours. Additional concentrated aqueous HCl added (3 drops) and mixture stirred overnight at 50° C. The mixture cooled to room temperature, diluted with EtOAc (2 mL) and suction filtered through sintered glass. Filtrate treated with saturated aqueous Na₂CO₃ until pH˜8-9 and mixture heated to 60° C. for 10 minutes, then cooled to room temperature. EtOH added (5 mL) and the red solution was collected and concentrated. Residue purified by preparative HPLC. Yield: 21%. MS (m/z): 520.2 (MH⁺).

### (Z)-1-(2-((6,7-Difluoro-5-methoxy-2-(1,3,5-trimethyl-1H-pyrazol-4-yl)-1H-indol-3-yl)methylene)-3-oxo-2,3-dihydrobenzofuran-5-yl)-3-methylurea MS (m/z): 508.2 (MH⁺)

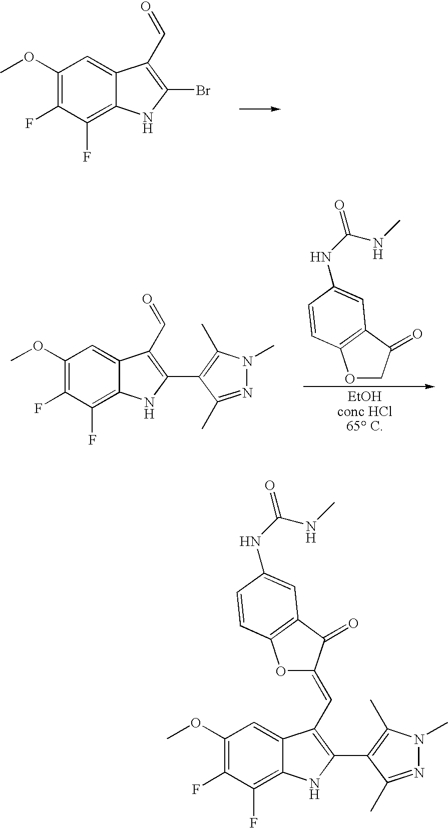

### Preparation of 2-bromo-6,7-difluoro-5-methoxy-1H-indole-3-carbaldehyde

Prepared via Gassman oxindole-Vilsmeier-Haack reactions using 2,3-difluoro-4-methoxyaniline. MS (m/z): 288.2 (MH−).

### Preparation of 6,7-difluoro-5-methoxy-2-(1,3,5-trimethyl-1H-pyrazol-4-yl)-1H-indole-3-carbaldehyde

Preparation via the Suzuki coupling method using 2-bromo-6,7-difluoro-5-methoxy-1H-indole-3-carbaldehyde. MS (m/z): 320.3 (MH⁺).

### Preparation of (Z)-1-(2-((6,7-difluoro-5-methoxy-2-(1,3,5-trimethyl-1H-pyrazol-4-yl)-1H-indol-3-yl)methylene)-3-oxo-2,3-dihydrobenzofuran-5-yl)-3-methylurea

Concentrated aqueous HCl (6 drops) was added to a stirred mixture of 6,7-difluoro-5-methoxy-2-(1,3,5-trimethyl-1H-pyrazol-4-yl)-1H-indole-3-carbaldehyde (89 mg, 0.28 mmol) and 1-methyl-3-(3-oxo-2,3-dihydrobenzofuran-5-yl)urea (52 mg, 0.25 mmol) in EtOH (2 mL). Resulting mixture stirred at 65° C. for 5 hours, and then sat overnight at room temperature. The mixture treated with EtOAc (2 mL) and filtered through sintered glass. Solid washed with 50% EtOH-EtOAc (3×2 mL) to give a yellow-orange solid that was collected and dried in vacuo. Yield: 51%. MS (m/z): 508.2 (MH⁺).

### (Z)-1-(2-((5-Methoxy-7-(trifluoromethyl)-2-(1,3,5-trimethyl-1H-pyrazol-4-yl)-1H-indol-3-yl)methylene)-3-oxo-2,3-dihydrobenzofuran-5-yl)-3-methyl urea MS (m/z): 540.2 (M H⁺)

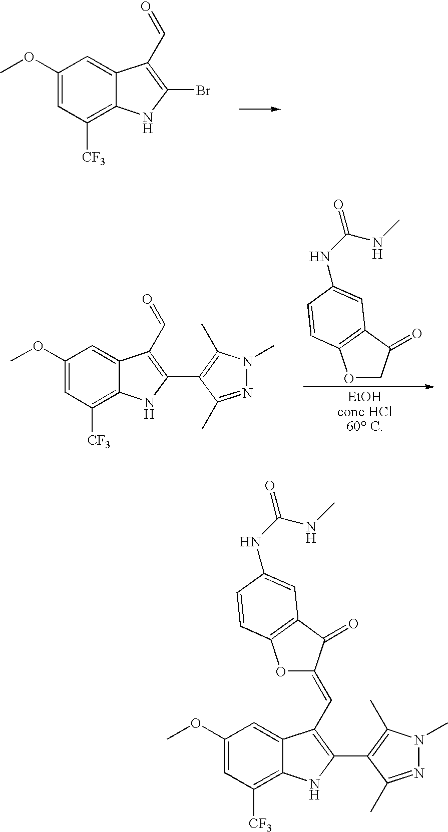

### Preparation of 2-bromo-5-methoxy-7-(trifluoromethyl)-1H-indole-3-carbaldehyde

Prepared via Gassman oxindole-Vilsmeier-Haack reactions using 4-methoxy-2-(trifluoromethyl)aniline. MS (m/z): 320.2 (MH−).

### Preparation of 5-methoxy-7-(trifluoromethyl)-2-(1,3,5-trimethyl-1H-pyrazol-4-yl)-1H-indole-3-carbaldehyde

Preparation via the Suzuki coupling method using 2-bromo-5-methoxy-7-(trifluoromethyl)-1H-indole-3-carbaldehyde. MS (m/z): 352.3 (MH⁺).

### Preparation of (Z)-1-(2-((5-methoxy-7-(trifluoromethyl)-2-(1,3,5-trimethyl-1H-pyrazol-4-yl)-1H-indol-3-yl)methylene)-3-oxo-2,3-dihydrobenzofuran-5-yl)-3-methylurea

Concentrated aqueous HCl (6 drops) was added to a stirred mixture of 5-methoxy-7-(trifluoromethyl)-2-(1,3,5-trimethyl-1H-pyrazol-4-yl)-1H-indole-3-carbaldehyde (96 mg, 0.27 mmol) and 1-methyl-3-(3-oxo-2,3-dihydrobenzofuran-5-yl)urea (56 mg, 0.27 mmol) in EtOH (2 mL). Resulting mixture stirred at 60° C. for 5 hours, and then 45° C. overnight. The mixture cooled to room temperature and EtOAc added (2 mL). The mixture suction filtered through sintered glass and resulting solid washed with 20% EtOH-EtOAc (3 mL). The tan solid dried in vacuo. Yield: 34%. MS (m/z): 540.2 (MH⁺).

### (Z)-1-(2-((5-methoxy-7-methyl-2-(1,3,5-trimethyl-1H-pyrazol-4-yl)-1H-indol-3-yl)methylene)-3-oxo-2,3-dihydrobenzofuran-5-yl)-3-methyl urea MS (m/z): 486.2 (M H⁺)

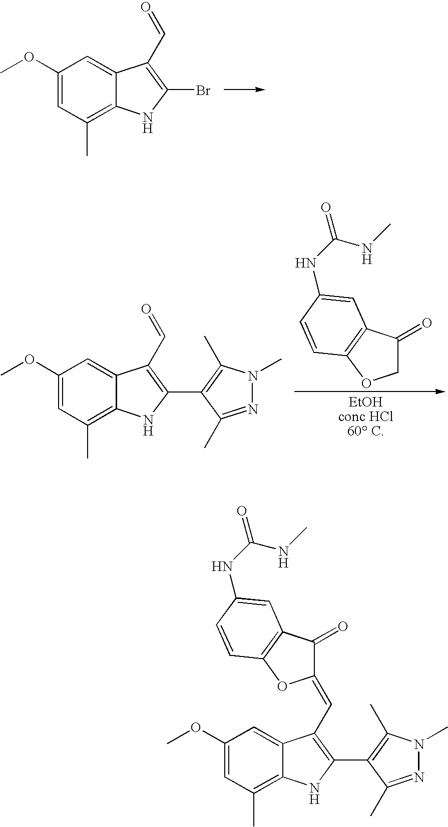

### Preparation of 2-bromo-5-methoxy-7-methyl-1H-indole-3-carbaldehyde

Prepared via Gassman oxindole-Vilsmeier-Haack reactions using 4-methoxy-2-methylaniline. MS (m/z): 268.2 (MH⁺).

### Preparation of 5-methoxy-7-methyl-2-(1,3,5-trimethyl-1H-pyrazol-4-yl)-1H-indole-3-carbaldehyde

Preparation via the Suzuki coupling method using 2-bromo-5-methoxy-7-methyl-1H-indole-3-carbaldehyde. MS (m/z): 298.3 (MH⁺).

### Preparation of (Z)-1-(2-((5-methoxy-7-methyl-2-(1,3,5-trimethyl-1H-pyrazol-4-yl)-1H-indol-3-yl)methylene)-3-oxo-2,3-dihydrobenzofuran-5-yl)-3-methylurea

Concentrated aqueous HCl (6 drops) was added to a stirred mixture of 5-methoxy-7-methyl-2-(1,3,5-trimethyl-1H-pyrazol-4-yl)-1H-indole-3-carbaldehyde (85 mg, 0.29 mmol) and 1-methyl-3-(3-oxo-2,3-dihydrobenzofuran-5-yl)urea (59 mg, 0.29 mmol) in EtOH (2 mL). The mixture stirred at 60° C. for 5 hours, and then 45° C. overnight. The mixture cooled to room temperature, diluted with EtOH (3 mL), and treated with saturated aqueous Na₂CO₃ (3 mL). Resulting mixture sonicated for 2-3 minutes, filtered, and filtrate concentrated. Residue treated with 25% EtOH-EtOAc and filtered. Filtrate sat overnight. An orange solid precipitated from the filtrate that was collected and dried in vacuo. Yield: 14%. MS (m/z): 486.2 (MH⁺).

### (Z)-1-(2-((2-(3,5-Dimethyl-1H-pyrazol-4-yl)-7-fluoro-5-methoxy-1H-indol-3-yl)methylene)-3-oxo-2,3-dihydrobenzofuran-5-yl)-3-methylurea MS (m/z): 476.2 (MH⁺)

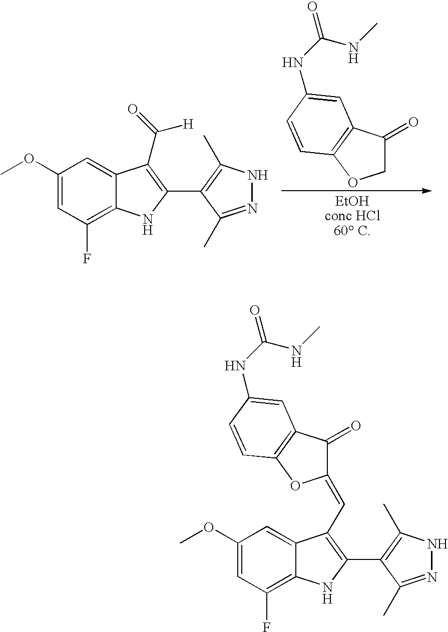

Preparation via the Suzuki coupling method using 2-bromo-7-fluoro-5-methoxy-1H-indole-3-carbaldehyde. MS (m/z): 288.3 (MH⁺).

### (Z)-1-(2-((7-Fluoro-2-(1-isobutyl-1H-pyrazol-4-yl)-5-methoxy-1H-indol-3-yl)methylene)-3-oxo-2,3-dihydrobenzofuran-5-yl)-3-methylurea MS (m/z): 504.2 (MH⁺)

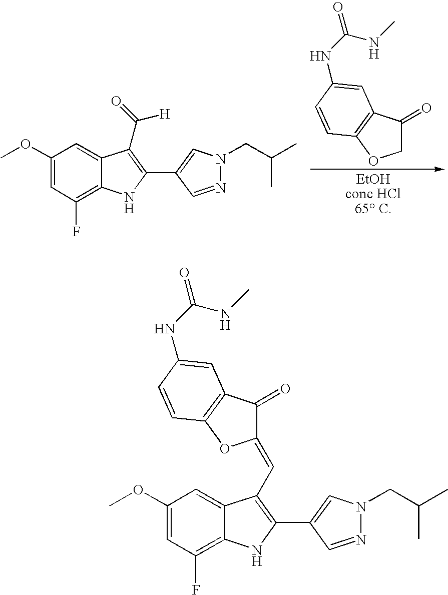

### Preparation of 7-fluoro-2-(1-isobutyl-1H-pyrazol-4-yl)-5-methoxy-1H-indole-3-carbaldehyde

Suzuki coupling method using 2-bromo-7-fluoro-5-methoxy-1H-indole-3-carbaldehyde. MS (m/z): 316.3 (MH⁺).

### Preparation of (Z)-1-(2-((7-fluoro-2-(1-isobutyl-1H-pyrazol-4-yl)-5-methoxy-1H-indol-3-yl)methylene)-3-oxo-2,3-dihydrobenzofuran-5-yl)-3-methylurea

Concentrated aqueous HCl (6 drops) was added to a stirred mixture of 7-fluoro-2-(1-isobutyl-1H-pyrazol-4-yl)-5-methoxy-1H-indole-3-carbaldehyde (80 mg, 0.25 mmol) and 1-methyl-3-(3-oxo-2,3-dihydrobenzofuran-5-yl)urea (47 mg, 0.23 mmol) in EtOH (2 mL). Resulting mixture stirred at 65° C. for 5 hours, and then 45° C. overnight. The mixture cooled to room temperature, diluted with EtOH (5 mL), and treated with saturated aqueous Na₂CO₃ (3 mL). Organic portion collected and concentrated. Resulting residue taken up in MeOH (5 mL) and filtered. Filtrate triturated with EtOAc until solid material was observed. The mixture let sit overnight. Mother liquor was collected from the solid and concentrated and resulting material purified by preparative HPLC. Yield: 46%. MS (m/z): 504.2 (MH⁺).

### (Z)-1-(2-((7-Fluoro-5-methoxy-2-(1-methyl-1H-pyrazol-4-yl)-1H-indol-3-yl)methylene)-3-oxo-2,3-dihydrobenzofuran-5-yl)-3-methylurea MS (m/z): 462.2 (MH⁺)

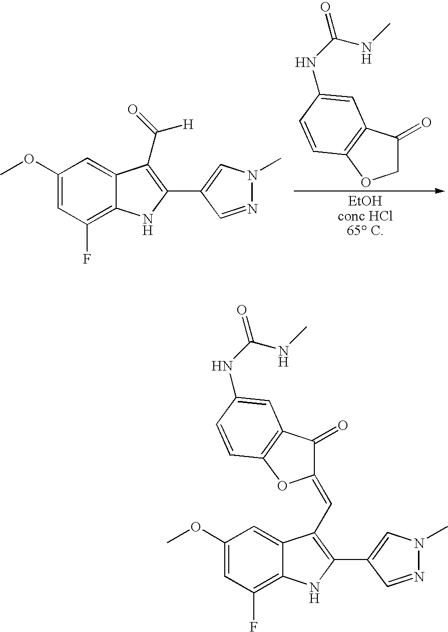

### Preparation of 7-fluoro-5-methoxy-2-(1-methyl-1H-pyrazol-4-yl)-1H-indole-3-carbaldehyde

Suzuki coupling method using 2-bromo-7-fluoro-5-methoxy-1H-indole-3-carbaldehyde. MS (m/z): 274.2 (MH⁺).

### Preparation of (Z)-1-(2-((7-fluoro-5-methoxy-2-(1-methyl-1H-pyrazol-4-yl)-1H-indol-3-yl)methylene)-3-oxo-2,3-dihydrobenzofuran-5-yl)-3-methylurea

Concentrated aqueous HCl (6 drops) was added to a stirred mixture of 7-fluoro-5-methoxy-2-(1-methyl-1H-pyrazol-4-yl)-1H-indole-3-carbaldehyde (100 mg, 0.37 mmol) and 1-methyl-3-(3-oxo-2,3-dihydrobenzofuran-5-yl)urea (68 mg, 0.33 mmol) in EtOH (2.5 mL). Resulting mixture stirred at 65° C. for 5 hours, and then 45° C. overnight. The mixture cooled to room temperature, diluted with EtOAc (2 mL) and filtered through sintered glass. Dark brown solid treated with DMSO (4 mL) and filtered through sintered glass. DMSO solution poured into water (20 mL) and resulting orange solid filtered. Orange solid washed with EtOH (10 mL) and filtered. Solid dried in vacuo. Yield: 32%. MS (m/z): 462.2 (MH⁺).

### N-[2-(Dimethylamino)ethyl]-4-({[(2Z)-2-{[7-fluoro-5-methoxy-2-(1,3,5-trimethyl-1H-pyrazol-4-yl)-1 indol-3-yl]methylidene}-3-oxo-2,3-dihydro-1-benzofuran-5-yl]carbamoyl}amino)-N-methylbenzamide MS (m/z): 680.2 (MH⁺)

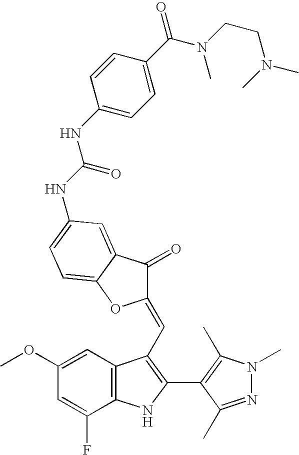

### Synthetic Scheme:

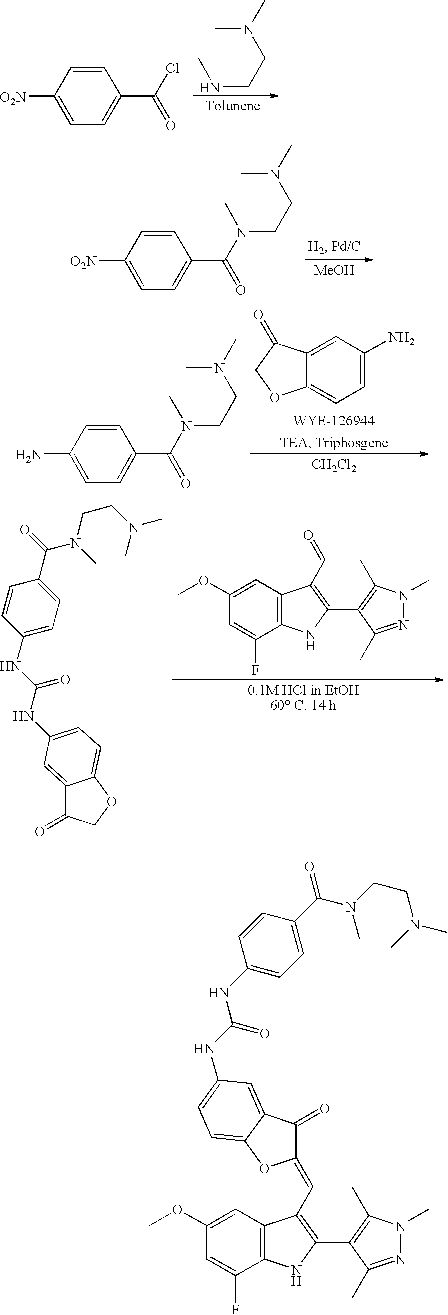

### N-(2-(Dimethylamino)ethyl)-N-methyl-4-nitrobenzamide

Into a solution of 4-nitrobenzoyl chloride (12 g, 64.7 mmol) in toluene (200 ml) was added in drops N1,N1,N2-trimethylethane-1,2-diamine (10.09 mL, 78 mmol). The reaction mixture was vigorously stirred at room temperature for 14 hours, then suction filtered. The solid was partitioned between ethyl acetate and saturated NaHCO₃ aqueous solution. The organic layer was washed with saturated NaCl aqueous solution, dried over MgSO₄, suction filtered, concentrated and dried further in vacuo to give N-(2-(dimethylamino)ethyl)-N-methyl-4-nitrobenzamide (9.2 g, 36.6 mmol, 56.6%) as a white solid. MS (m/z): 252.2 (MH⁺)

### 4-Amino-N-(2-(dimethylamino)ethyl)-N-methylbenzamide

Into an solution of N-(2-(dimethylamino)ethyl)-N-methyl-4-nitrobenzamide (4 g, 15.92 mmol) in methanol (50 ml) was added Pd/C 10% (1 g, 0.940 mmol). The reaction flask was sealed with a rubber septa and a 2 L balloon of hydrogen gas was inserted. The reaction mixture was stirred under the hydrogen balloon pressure at room temperature for 14 hours. The resulting reaction mixture was suction filtered through a Celite™ bed. The filtrate was concentrated and dried further in vacuo to give 3.5 g of the desired product 4-amino-N-(2-(dimethylamino)ethyl)-N-methylbenzamide (3.5 g, 15.82 mmol, 99%) as a colorless gel. MS (m/z): 222.2 (MH⁺)

### N-[2-(Dimethylamino)ethyl]-N-methyl-4-{[(3-oxo-2,3-dihydro-1-benzofuran-5-yl)carbamoyl]amino}benzamide TFA salt

Into as solution of 5-aminobenzofuran-3(2H)-one (1 g, 6.70 mmol) in dichloromethane (50 ml) was added triethylamine (0.890 mL, 6.70 mmol) followed by an addition of triphosgene (0.657 g, 2.213 mmol) in dichloromethane solution (10 ml). The mixture was stirred for 1 hour and 4-amino-N-(2-(dimethylamino)ethyl)-N-methylbenzamide (1.484 g, 6.70 mmol) in dichloromethane (20 ml) was added. The reaction mixture was stirred at room temperature for 14 hours, then diluted with methanol and suction filtered. The filtrate was concentrated, re-dissolved with DMSO (10 ml) and suction filtered. The DMSO filtrate was purified by HPLC to give the desired product N-[2-(dimethylamino)ethyl]-N-methyl-4-{[(3-oxo-2,3-dihydro-1-benzofuran-5-yl)carbamoyl]amino}benzamide TFA salt (1.28 g, 2.508 mmol, 37.4%) as a light yellow solid. MS (m/z): 397.2 (MH⁺)

### (Z)-N-(2-(Dimethylamino)ethyl)-4-(3-(2-((7-fluoro-5-methoxy-2-(1,3,5-trimethyl-1H-pyrazol-4-yl)-1H-indol-3-yl)methylene)-3-oxo-2,3-dihydrobenzofuran-5-yl)ureido)-N-methylbenzamide TFA salt

A mixture of N-(2-(dimethylamino)ethyl)-N-methyl-4-(3-(3-oxo-2,3-dihydrobenzofuran-5-yl)ureido)benzamide TFA salt (2.4 g, 4.70 mmol) and 7-fluoro-5-methoxy-2-(1,3,5-trimethyl-1H-pyrazol-4-yl)-1H-indole-3-carbaldehyde (1.417 g, 4.70 mmol) in 0.1M HCl solution in ethanol (100 ml) was stirred at 60° C. for 18 hours, then concentrated. The residue was purified by HPLC (0.1% TFA) to give N-[2-(dimethylamino)ethyl]-4-({[(2Z)-2-{[7-fluoro-5-methoxy-2-(1,3,5-trimethyl-1H-pyrazol-4-yl)-1H-indol-3-yl]methylidene}-3-oxo-2,3-dihydro-1-benzofuran-5-yl]carbamoyl}amino)-N-methylbenzamide TFA salt (1.58 g, 1.931 mmol, 41.1%) as an orange solid. MS (m/z): 680.2 (MH⁺)

The following compounds were synthesized using the procedure above.

### N-[2-(Dimethylamino)ethyl]-4-({[(2Z)-2-{[7-fluoro-5-methoxy-2-(1,3,5-trimethyl-1H-pyrazol-4-yl)-1H-indol-3-yl]methylidene}-3-oxo-2,3-dihydro-1-benzofuran-5-yl]carbamoyl}amino)benzamide MS (m/z): 666.4 (MH⁺)

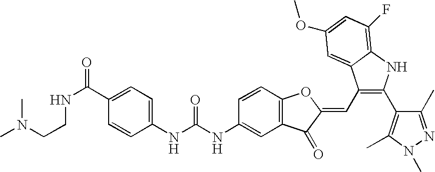

### 1-(4-{[3-(Dimethylamino)pyrrolidin-1-yl]carbonyl}phenyl)-3-[(2Z)-2-{[7-fluoro-5-methoxy-2-(1,3,5-trimethyl-1H-pyrazol-4-yl)-1H-indol-3-yl]methylidene}-3-oxo-2,3-dihydro-1-benzofuran-5-yl]urea MS (m/z): 692.4 (MH⁺)

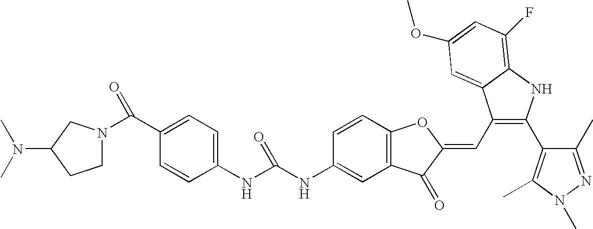

### 1-{4-[(3,4-Dimethylpiperazin-1-yl)carbonyl]phenyl}-3-[(2Z)-2-{[7-fluoro-5-methoxy-2-(1,3,5-trimethyl-1H-pyrazol-4-yl)-1H-indol-3-yl]methylidene}-3-oxo-2,3-dihydro-1-benzofuran-5-yl]urea MS (m/z): 692.3 (MH⁺)

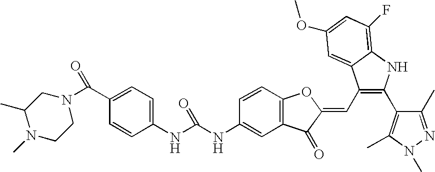

### 1-(4-{[4-(Dimethylamino)piperidin-1-yl]carbonyl}phenyl)-3-[(2Z)-2-{[7-fluoro-5-methoxy-2-(1,3,5-trimethyl-1H-pyrazol-4-yl)-1H-indol-3-yl]methylidene}-3-oxo-2,3-dihydro-1-benzofuran-5-yl]urea MS (m/z): 706.5 (MH⁺)

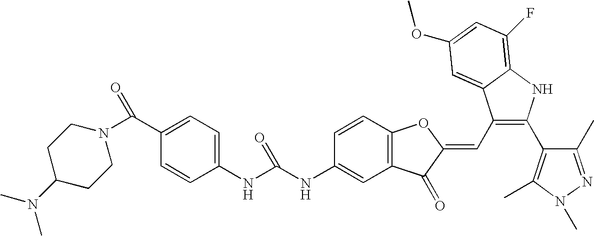

### 1-[(2Z)-2-{[7-Fluoro-5-methoxy-2-(1,3,5-trimethyl-1H-pyrazol-4-yl)-1H-indol-3-yl]methylidene}-3-oxo-2,3-dihydro-1-benzofuran-5-yl]-3-[4-(morpholin-4-ylcarbonyl)phenyl]urea MS (m/z): 665.4 (MH⁺)

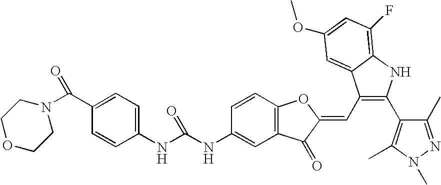

### (1-[(2Z)-2-{[7-Fluoro-5-methoxy-2-(1,3,5-trimethyl-1H-pyrazol-4-yl)-1H-indol-3-yl]methylidene}-3-oxo-2,3-dihydro-1-benzofuran-5-yl]-3-(4-{[4-(2-hydroxyethyl)piperazin-1-yl]carbonyl}phenyl)urea MS (m/z): 708.2 (MH⁺)

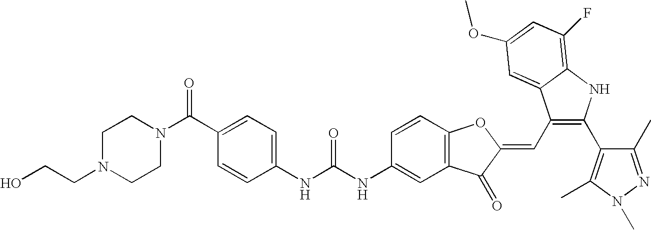

### N-[2-(Dimethylamino)ethyl]-4-({[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]carbamoyl}amino)-N-methylbenzamide

MS (m/z): 662.4 (MH⁺)

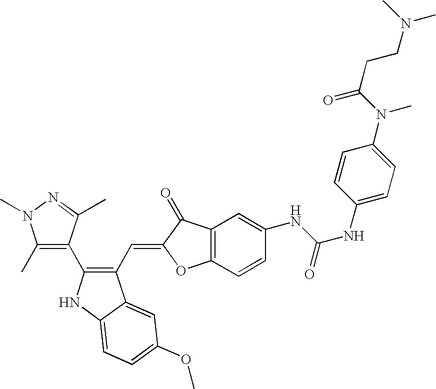

### Synthetic Scheme:

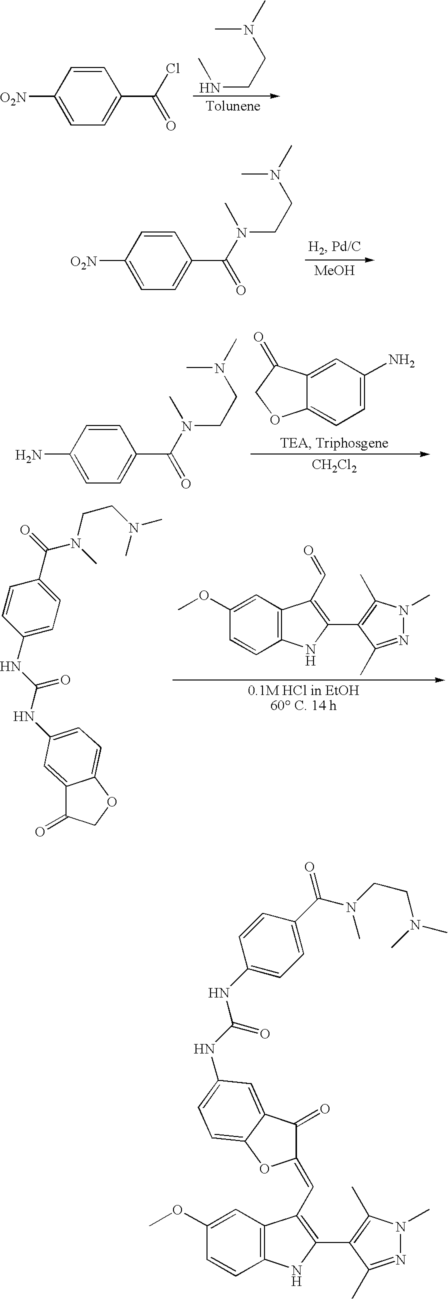

### N-[2-(Dimethylamino)ethyl]-4-({[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]carbamoyl}amino)benzamide MS (m/z): 648.3 (MH⁺)

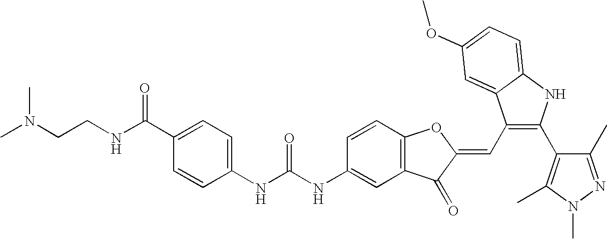

### 4-({[(2Z)-2-{[5-Methoxy-2-(1,3,5-trimethyl-1H-pyrazol-4-yl)-1H-indol-3-yl]methylidene}-3-oxo-2,3-dihydro-1-benzofuran-5-yl]carbamoyl}amino)-N,N-dimethyl benzamide MS (m/z): 605.3 (MH⁺)

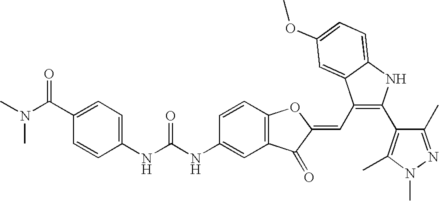

### 1-{4-[(3,4-Dimethylpiperazin-1-yl)carbonyl]phenyl}-3-[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]urea MS (m/z): 674.3 (MH⁺)

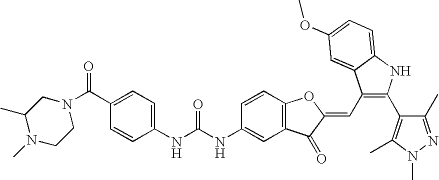

### 4-({[(2Z)-2-{[5-Methoxy-2-(1,3,5-trimethyl-1H-pyrazol-4-yl)-1H-indol-3-yl]methylidene}-3-oxo-2,3-dihydro-1-benzofuran-5-yl]carbamoyl}amino)-N-methyl-N-(2-pyrrolidin-1-ylethyl)benzamide MS (m/z): 688.3 (MH⁺)

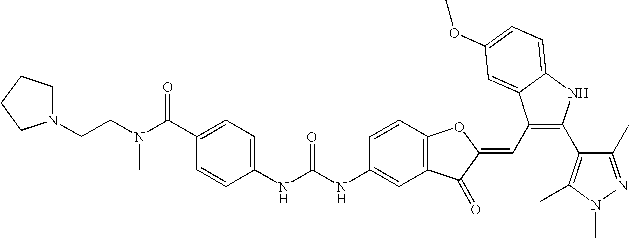

### 1-(4-{[4-(Dimethylamino)piperidin-1-yl]carbonyl}phenyl)-3-[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]urea MS (m/z): 688.5 (MH⁺)

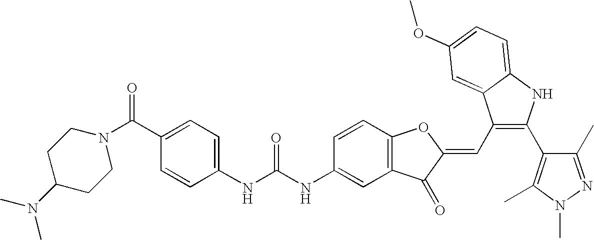

### 1-[(2Z)-2-{[5-Methoxy-2-(1,3,5-trimethyl-1H-pyrazol-4-yl)-1H-indol-3-yl]methylidene}-3-oxo-2,3-dihydro-1-benzofuran-5-yl]-3-(4-{[4-(1-methylethyl)piperazin-1-yl]carbonyl}phenyl)urea MS (m/z): 688.3 (MH⁺)

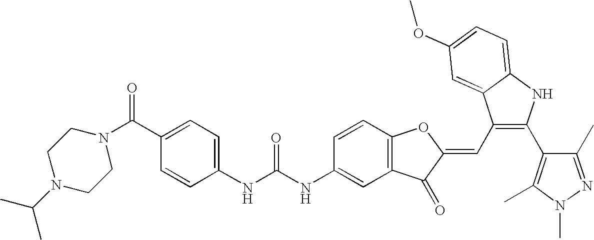

### 4-({[(2Z)-2-{[5-Methoxy-2-(1,3,5-trimethyl-1H-pyrazol-4-yl)-1H-indol-3-yl]methylidene}-3-oxo-2,3-dihydro-1-benzofuran-5-yl]carbamoyl}amino)-N-methyl-N-(1-methylpyrrolidin-3-yl)benzamide MS (m/z): 674.2 (MH⁺)

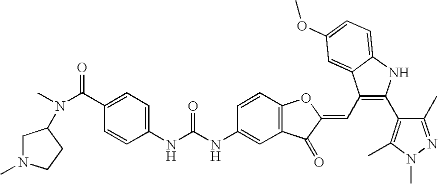

### 1-{4-[(4-Ethyl piperazin-1-yl)carbonyl]phenyl}-3-[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]urea MS (m/z): 674.2 (MH⁺)

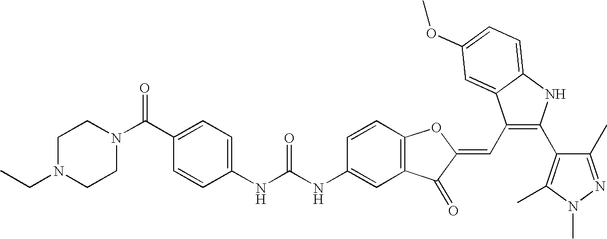

### 1-(4-{[3-(Dimethylamino)pyrrolidin-1-yl]carbonyl}phenyl)-3-[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]urea MS (m/z): 674.1 (MH⁺)

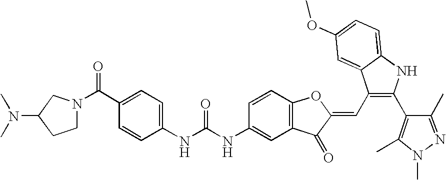

### 1-[(2Z)-2-{[5-Methoxy-2-(1,3,5-trimethyl-1H-pyrazol-4-yl)-1H-indol-3-yl]methylidene}-3-oxo-2,3-dihydro-1-benzofuran-5-yl]-3-[4-(morpholin-4-ylcarbonyl)phenyl]urea MS (m/z): 647.3 (MH⁺)

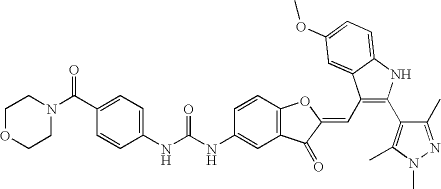

### 1-{4-[(1,1-Dioxidothiomorpholin-4-yl)carbonyl]phenyl}-3-[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]urea MS (m/z): 695.1 (MH⁺)

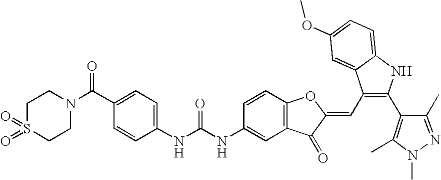

### 1-[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]-3-{4-[(4-methylpiperazin-1-yl)carbonyl]phenyl}urea MS (m/z): 660.1 (MH⁺).

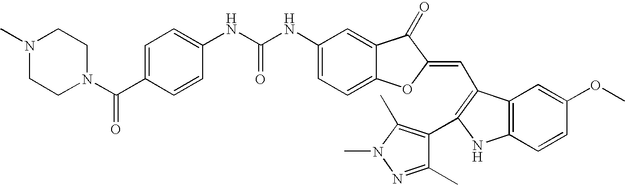

### 4-[({(2Z)-2-[(2-Cyclohexyl-5-methoxy-1H-indol-3-yl)methylidene]-3-oxo-2,3-dihydro-1-benzofuran-5-yl}carbamoyl)amino]-N-[2-(dimethylamino)ethyl]-N-methyl benzamide MS (m/z): 636.4 (MH⁺)

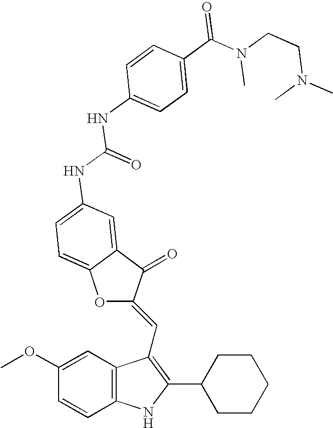

### Synthetic Scheme:

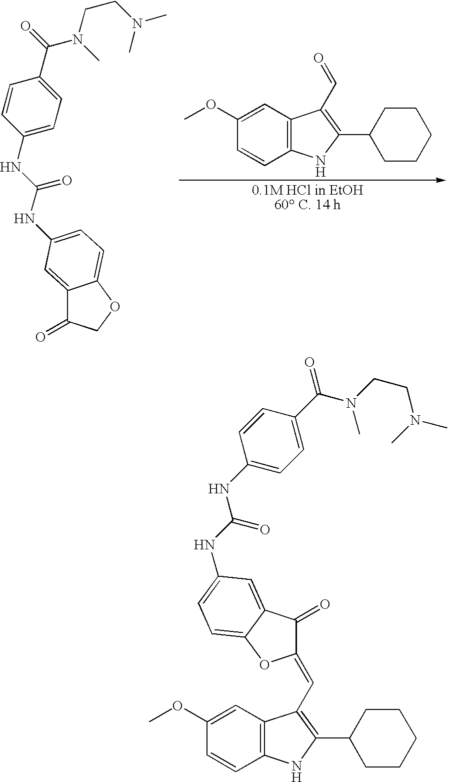

### 1-{(2Z)-2-[(2-Cyclohexyl-5-methoxy-1H-indol-3-yl)methylene]-3-oxo-2,3-dihydro-1-benzofuran-5-yl}-3-{4-[(4-methylpiperazin-1-yl)carbonyl]phenyl}urea MS (m/z): 634.3 (MH⁺)

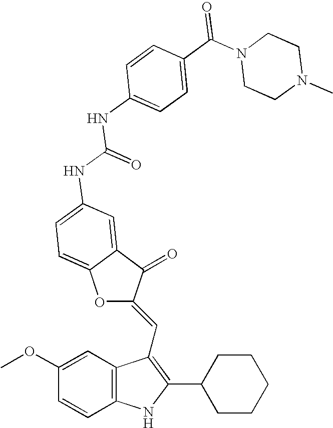

### 1-{(2Z)-2-[(2-Cyclohexyl-1-ethyl-5-methoxy-1H-indol-3-yl)methylene]-3-oxo-2,3-dihydro-1-benzofuran-5-yl}-3-{4-[(4-methylpiperazin-1-yl)carbonyl]phenyl}urea MS (m/z): 662.4 (M H⁺)

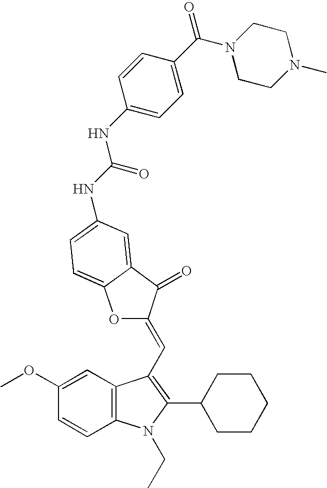

### Synthetic Scheme:

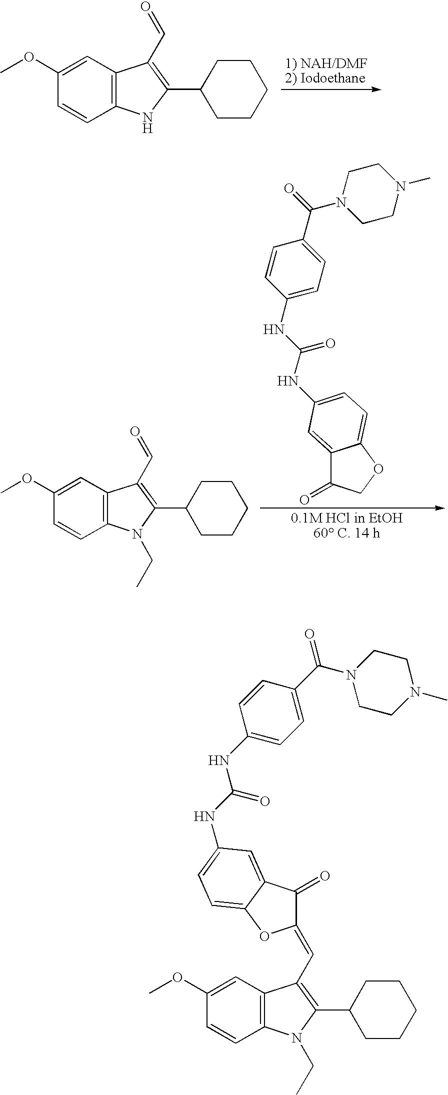

### Step 1: 2-Cyclohexyl-1-ethyl-5-methoxy-1H-indole-3-carbaldehyde

Into a solution of 2-cyclohexyl-5-methoxy-1H-indole-3-carbaldehyde (128.6 mg, 0.5 mmol) in DMF (10 mL) was added NaH (40 mg, 1.0 mmol). The mixture was stirred at room temperature for 30 minutes and iodoethane (389 mg, 2.5 mmol) was added. The reaction mixture was stirred at room temperature for 2 hours, then partitioned between water and ethyl acetate. The organic layer was washed with saturated NaCl aqueous solution, dried over MgSO₄, filtered, concentrated and chromatographed over a 40 g silica column (eluting with hexanes:ethyl acetate 1:1) to provide the desired product 2-cyclohexyl-1-ethyl-5-methoxy-1H-indole-3-carbaldehyde (107 mg, 0.35 mmol, 75%) as light yellow solid. MS (m/z): 286.2 (MH⁺)

### 1-{(2Z)-2-[(2-Cyclohexyl-5-methoxy-1H-indol-3-yl)methylene]-3-oxo-2,3-dihydro-1-benzofuran-5-yl}-3-methylurea MS (m/z): 446.2 (MH⁺)

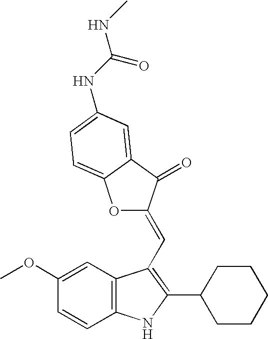

### Synthetic Scheme:

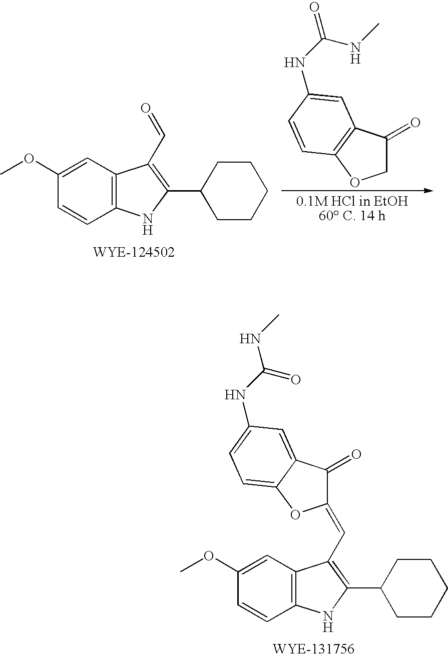

### 1-[4-(Dimethylamino)phenyl]-3-[(2Z)-2-({5-methoxy-2-methyl-1-[2-(4-methylpiperazin-1-yl)ethyl]-1H-indol-3-yl}methylidene)-3-oxo-2,3-dihydro-1-benzofuran-5-yl]urea MS (m/z): 609.4 (MH⁺)

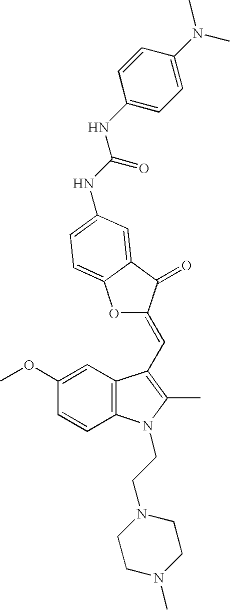

### Synthetic Scheme:

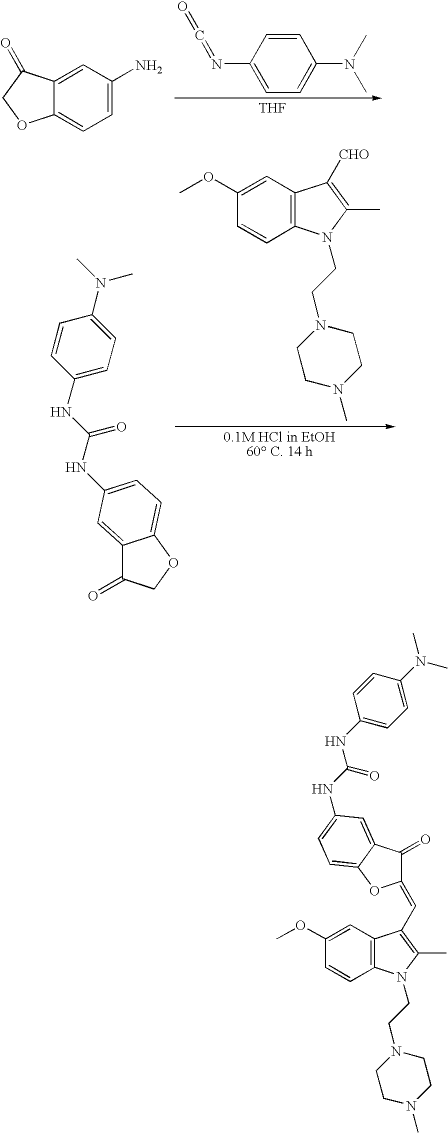

### 1-[4-(Dimethylamino)phenyl]-3-(3-oxo-2,3-dihydro-1-benzofuran-5-yl)urea

A mixture of 5-amino-1-benzofuran-3(2H)-one (280 mg, 1.88 mmol) and 4-(dimethylamino) phenyl isocyanate (304 mg, 1.88 mmol) and triethylamine (65 μL, 0.49 mmol) in THF (10 ml) was stirred at room temperature for 12 hours. The resulting reaction mixture was suction filtered and dried further in vacuo to provide 1-[4-(dimethylamino)phenyl]-3-(3-oxo-2,3-dihydro-1-benzofuran-5-yl)urea (357.5 mg, 61%) as a light yellow solid. MS (m/z): 312.2 (MH⁺)

### 1-[(2Z)-2-({5-Methoxy-2-methyl-1-[2-(4-methylpiperazin-1-yl)ethyl]-1H-indol-3-yl}methylidene)-3-oxo-2,3-dihydro-1-benzofuran-5-yl]-3-pyridin-3-yl urea MS (m/z): 567.3 (MH⁺)

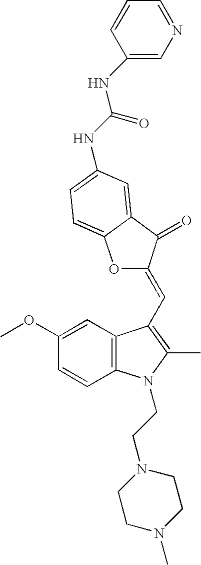

### 1-{(2Z)-2-[(2-{4-[(Dimethylamino)methyl]phenyl}-5-methoxy-1H-indol-3-yl)methylidene]-3-oxo-2,3-dihydro-1-benzofuran-5-yl}-3-methyl urea MS (m/z): 497.3 (MH⁺)

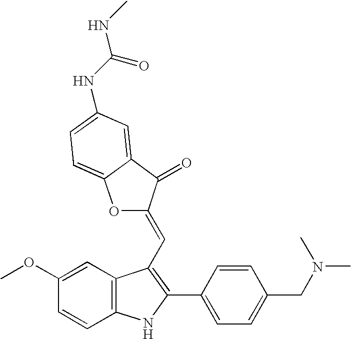

### 1-{(2Z)-2-[(2-{4-[(Dimethylamino)methyl]phenyl}-5-methoxy-1-[2-(4-methylpiperazin-1-yl)ethyl]-1H-indol-3-yl)methylidene]-3-oxo-2,3-dihydro-1-benzofuran-5-yl}-3-methyl urea MS (m/z): 312.2 (MH²⁺)

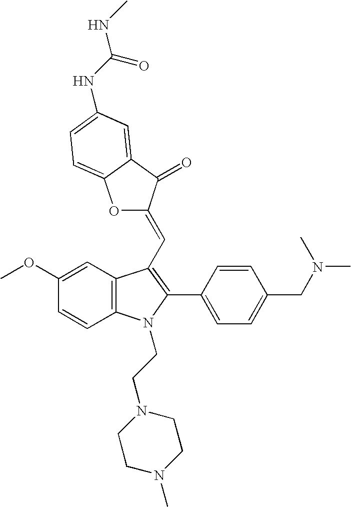

### 1-{(2Z)-2-[(2-{3-[(Dimethylamino)methyl]phenyl}-5-methoxy-1H-indol-3-yl)methylidene]-3-oxo-2,3-dihydro-1-benzofuran-5-yl}-3-methyl urea MS (m/z): 497.3 (MH⁺)

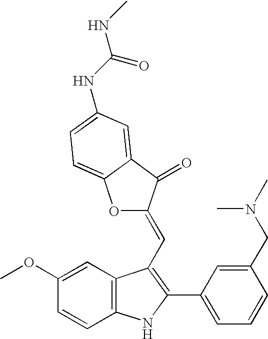

### 1-[(2Z)-2-({2-Cyclopentyl-5-methoxy-1-[2-(4-methylpiperazin-1-yl)ethyl]-1H-indol-3-yl}methylene)-3-oxo-2,3-dihydro-1-benzofuran-5-yl]-3-pyridin-3-yl urea MS (m/z): 311.2 (M2H⁺⁺)

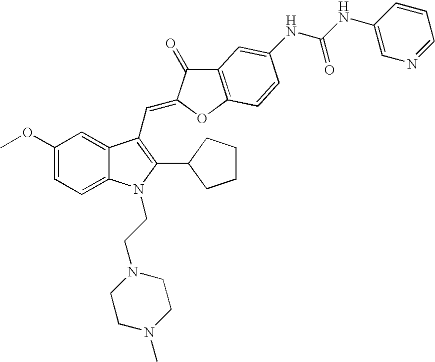

### 1-[(2Z)-2-({5-Methoxy-2-methyl-1-[2-(4-methylpiperazin-1-yl)ethyl]-1H-indol-3-yl}methylene)-3-oxo-2,3-dihydro-1-benzofuran-5-yl]-3-methylthiourea MS (m/z): 520.3 (MH⁺)

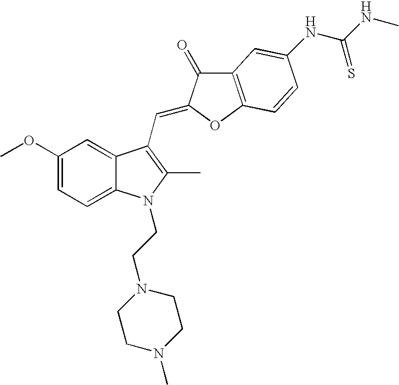

### 1-[(2Z)-2-{[7-Fluoro-5-methoxy-2-(1,3,5-trimethyl-1H-pyrazol-4-yl)-1H-indol-3-yl]methylidene}-3-oxo-2,3-dihydro-1-benzofuran-5-yl]-3-[2-(methylamino)ethyl]urea MS (m/z): 533.4 (MH⁺)

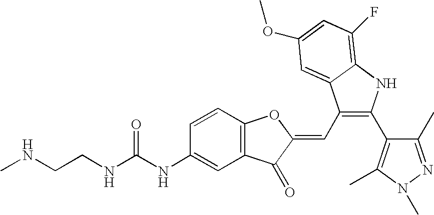

### Synthetic Scheme:

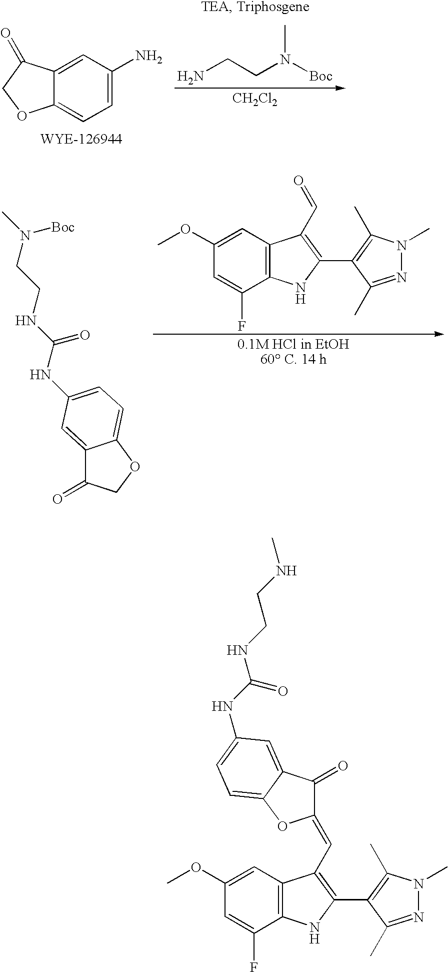

### tert-Butyl methyl(2-(3-(3-oxo-2,3-dihydrobenzofuran-5-yl)ureido)ethyl)carbamate

Into a solution of 5-aminobenzofuran-3(2H)-one (149 mg, 1 mmol) in THF (40 mL) was added triethylamine (139 μL, 1 mmol) followed by addition of triphosgene (98 mg, 0.330 mmol). The mixture was stirred at room temperature for 1 hour and tert-butyl 2-aminoethyl(methyl)carbamate (174 mg, 1.000 mmol) was added. The reaction mixture was stirred at room temperature for 12 hours, then concentrated. The residue was chromatograph over a 40 g of silica, eluting with ethyl acetate to provide tert-butyl methyl(2-(3-(3-oxo-2,3-dihydrobenzofuran-5-yl)ureido)ethyl)carbamate (148 mg, 0.424 mmol, 42.4%) as a beige solid. MS (m/z): 350.4 (MH⁺)

### 1-(2-Aminoethyl)-3-[(2Z)-2-{[7-fluoro-5-methoxy-2-(1,3,5-trimethyl-1H-pyrazol-4-yl)-1H-indol-3-yl]methylidene}-3-oxo-2,3-dihydro-1-benzofuran-5-yl]urea MS (m/z): 519.2 (MH⁺)

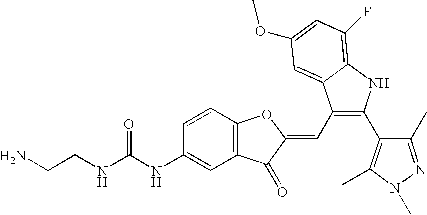

### 1-[2-(Dimethylamino)ethyl]-3-[(2Z)-2-{[7-fluoro-5-methoxy-2-(1,3,5-trimethyl-1H-pyrazol-4-yl)-1H-indol-3-yl]methylidene}-3-oxo-2,3-dihydro-1-benzofuran-5-yl]urea MS (m/z): 547.2 (MH⁺)

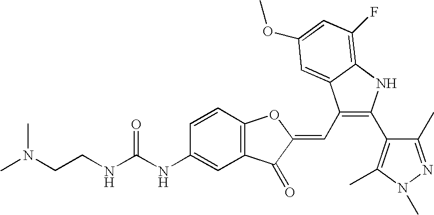

### 1-[(2Z)-2-{[7-Fluoro-5-methoxy-2-(1,3,5-trimethyl-1H-pyrazol-4-yl)-1H-indol-3-yl]methylidene}-3-oxo-2,3-dihydro-1-benzofuran-5-yl]-3-(2-pyrrolidin-1-ylethyl)urea MS (m/z): 573.6 (MH⁺)

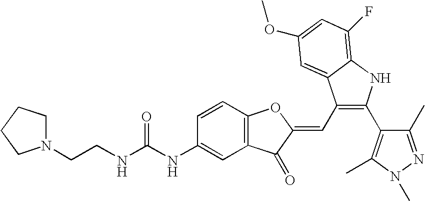

### N-[2-(Dimethylamino)ethyl]-3-({[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]carbamoyl}amino)-N-methylbenzamide MS (m/z): 662.3 (MH⁺)

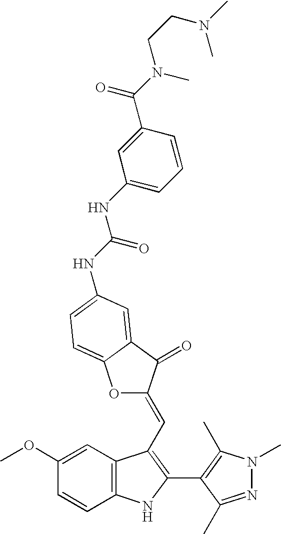

### N-[3-(Dimethylamino)propyl]-3-({[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]carbamoyl}amino)-N-methylbenzamide MS (m/z): 676.3 (MH⁺)

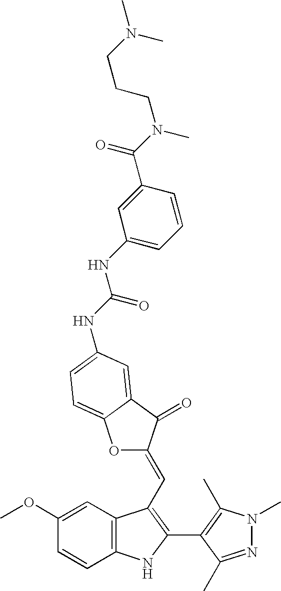

### N-[2-(Dimethylamino)ethyl]-4-({[(2Z)-2-({5-methoxy-2-methyl-1-[2-(4-methylpiperazin-1-yl)ethyl]-1H-indol-3-yl}methylene)-3-oxo-2,3-dihydro-1-benzofuran-5-yl]carbamoyl}amino)-N-methylbenzamide MS (m/z): 694.4 (MH⁺)

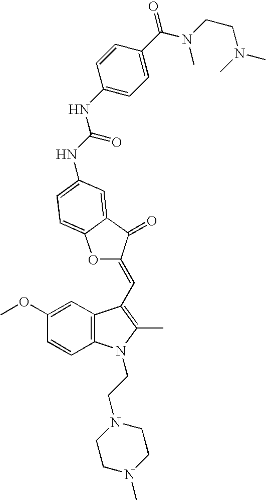

### 1-[(2Z)-2-({5-Methoxy-2-methyl-1-[2-(4-methylpiperazin-1-yl)ethyl]-1H-indol-3-yl}methylidene)-3-oxo-2,3-dihydro-1-benzofuran-5-yl]-3-[4-(morpholin-4-ylcarbonyl)phenyl]urea MS (m/z): 679.1 (MH⁺)

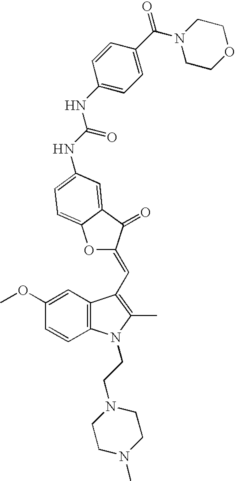

### 1-{(2Z)-2-[(1-Ethyl-5-methoxy-2-methyl-1H-indol-3-yl)methylidene]-3-oxo-2,3-dihydro-1-benzofuran-5-yl}-3-[4-(morpholin-4-ylcarbonyl)phenyl]urea MS (m/z): 581.1 (MH⁺)

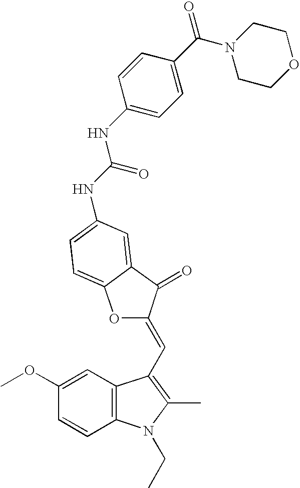

### 1-{(2Z)-2-[(5-Methoxy-1,2-dimethyl-1H-indol-3-yl)methylidene]-3-oxo-2,3-dihydro-1-benzofuran-5-yl}-3-{4-[(4-methylpiperazin-1-yl)carbonyl]phenyl}urea MS (m/z): 580.4 (MH⁺)

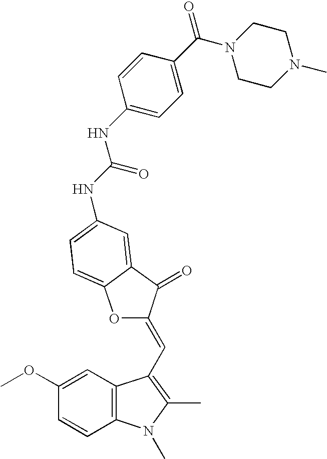

### N-[2-(Dimethylamino)ethyl]-4-[({(2Z)-2-[(1-ethyl-5-methoxy-1H-indol-3-yl)methylene]-3-oxo-2,3-dihydro-1-benzofuran-5-yl}carbamoyl)amino]-N-methyl benzamide MS (m/z): 582.3 (MH⁺)

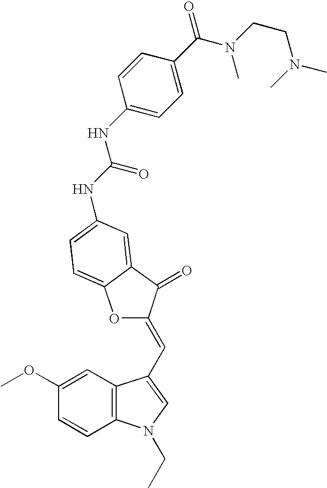

### 1-{(2Z)-2-[(1-Ethyl-5-methoxy-1H-indol-3-yl)methylene]-3-oxo-2,3-dihydro-1-benzofuran-5-yl}-3-{4-[(4-methylpiperazin-1-yl)carbonyl]phenyl}urea MS (m/z): 580.3 (MH⁺)

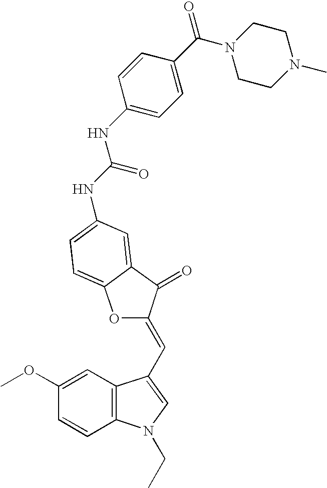

### 1-{(2Z)-2-[(5-Methoxy-1H-indol-3-yl)methylene]-3-oxo-2,3-dihydro-1-benzofuran-5-yl}-3-{4-[(4-methylpiperazin-1-yl)carbonyl]phenyl}urea MS (m/z): 552.3 (MH⁺)

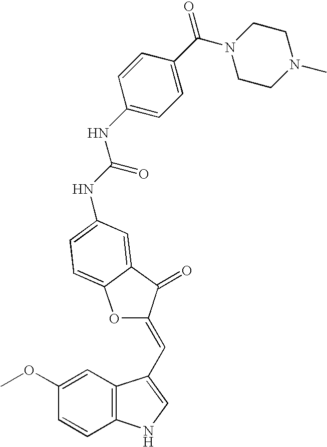

### N-[3-(Dimethylamino)propyl]-4-[({(2Z)-2-[(1-ethyl-5-methoxy-2-methyl-1H-indol-3-yl)methylidene]-3-oxo-2,3-dihydro-1-benzofuran-5-yl}carbamoyl)amino]benzamide MS (m/z): 596.2 (MH⁺)

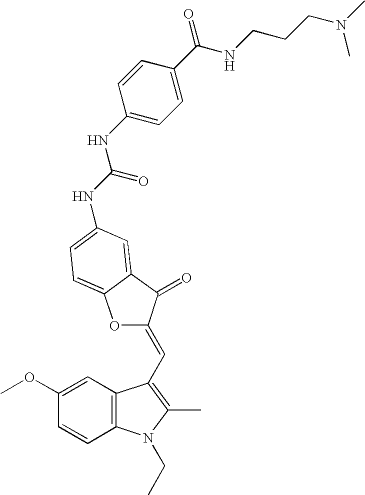

### N-[3-(Dimethylamino)propyl]-4-[({(2Z)-2-[(1-ethyl-5-methoxy-2-methyl-1H-indol-3-yl)methylene]-3-oxo-2,3-dihydro-1-benzofuran-5-yl}carbamoyl)amino]-N-methyl benzamide MS (m/z): 610.3 (MH⁺)

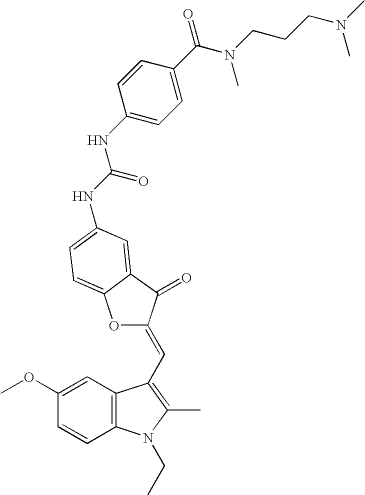

### N-[2-(Dimethylamino)ethyl]-4-[({(2Z)-2-[(1-ethyl-5-methoxy-2-methyl-1H-indol-3-yl)methylidene]-3-oxo-2,3-dihydro-1-benzofuran-5-yl}carbamoyl)amino]-N-methylbenzenesulfonamide: MS (m/z): 632.2 (MH⁺)

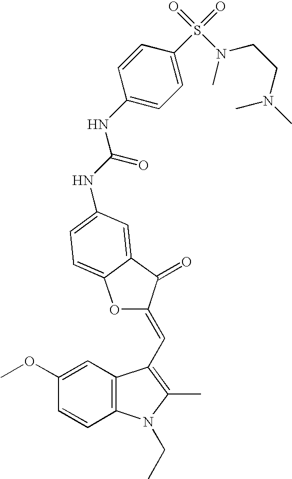

### Synthetic Scheme:

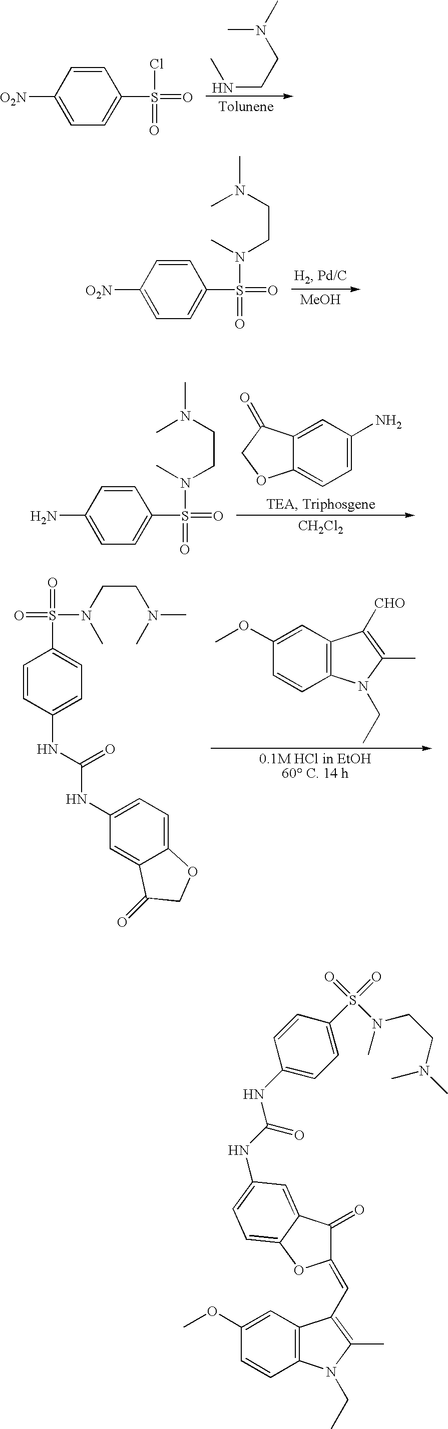

### 1-{(2Z)-2-[(1-Ethyl-5-methoxy-2-methyl-1H-indol-3-yl)methylidene]-3-oxo-2,3-dihydro-1-benzofuran-5-yl}-3-{4-[(4-methylpiperazin-1-yl)sulfonyl]phenyl}urea MS (m/z): 630.3 (MH⁺)

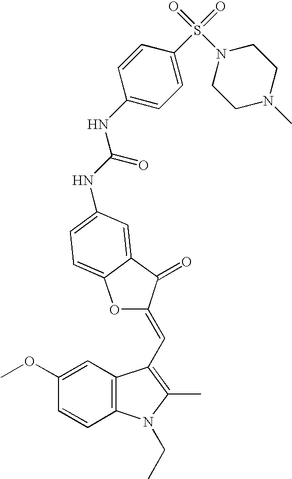

### 4-({[(2Z)-2-({5-Methoxy-2-methyl-1-[2-(4-methylpiperazin-1-yl)ethyl]-1H-indol-3-yl}methylidene)-3-oxo-2,3-dihydro-1-benzofuran-5-yl]carbamoyl}amino)-N-[2-(methylamino)ethyl]benzamide MS (m/z): 666.3 (MH⁺)

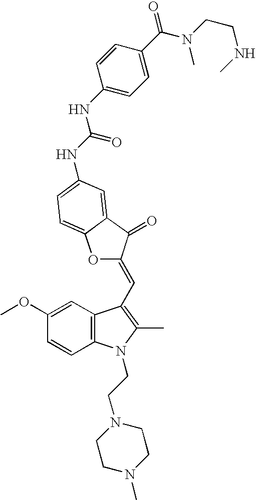

### Synthetic Scheme:

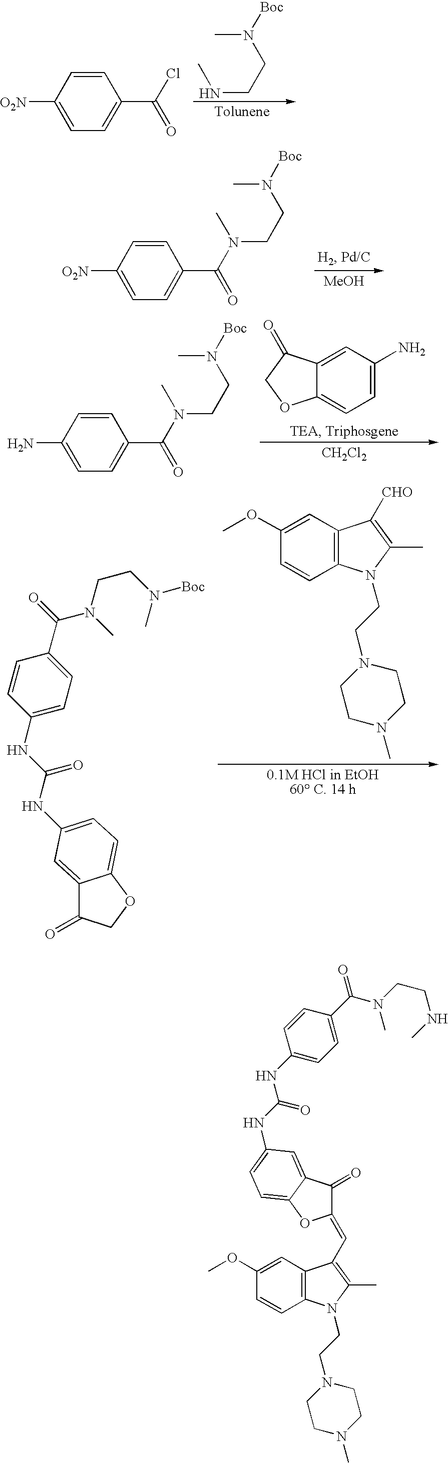

### 4-[({(2Z)-2-[(1-Ethyl-5-methoxy-2-methyl-1H-indol-3-yl)methylene]-3-oxo-2,3-dihydro-1-benzofuran-5-yl}carbamoyl)amino]-N-[2-(methylamino)ethyl]benzamide MS (m/z): 568.3 (MH⁺)

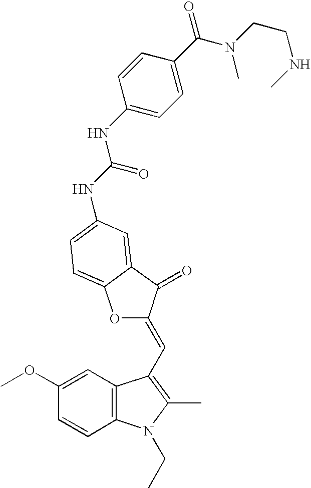

### 4-[({(2Z)-2-[(2-Cyclohexyl-5-methoxy-1H-indol-3-yl)methylene]-3-oxo-2,3-dihydro-1-benzofuran-5-yl}carbamoyl)amino]-N-[2-(methylamino)ethyl]benzamide: MS (m/z): 608.3 (MH⁺)

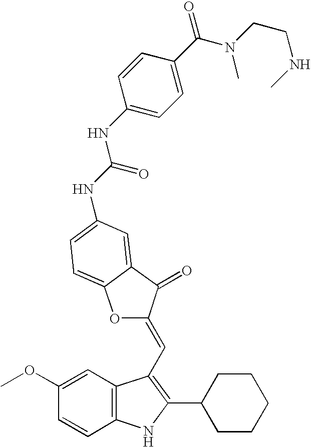

### 4-[({(2Z)-2-[(5-Methoxy-1,2-dimethyl-1H-indol-3-yl)methylene]-3-oxo-2,3-dihydro-1-benzofuran-5-yl}carbamoyl)amino]-N-[2-(methylamino)ethyl]benzamide MS (m/z): 554.3 (MH⁺)

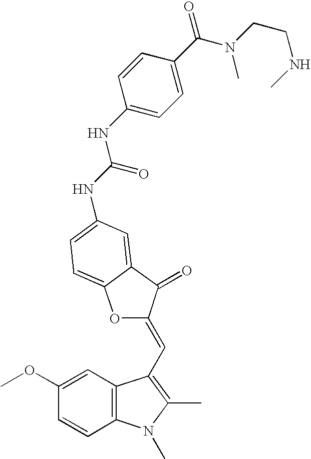

### N-[4-({[(2Z)-2-{[5-Methoxy-2-(1,3,5-trimethyl-1H-pyrazol-4-yl)-1H-indol-3-yl]methylidene}-3-oxo-2,3-dihydro-1-benzofuran-5-yl]carbamoyl}amino)phenyl]-N3,N3-dimethyl-beta-alaninamide MS (m/z): 648.1 (MH⁺)

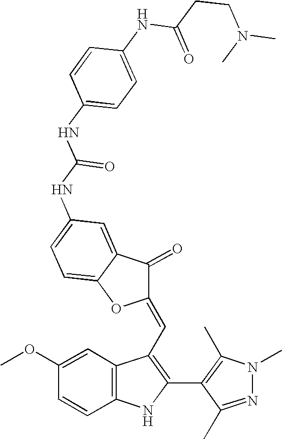

### Synthetic Scheme:

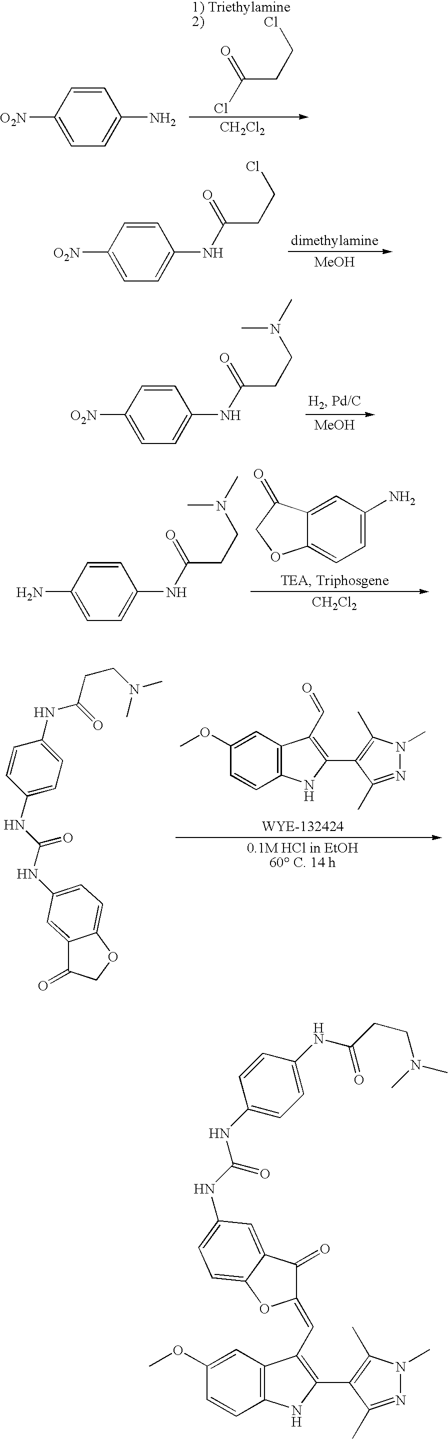

### 3-Chloro-(4-nitrophenyl)propanamide

Into a solution of 4-nitroaniline (1.38 g, 10 mmol) in dichloromethane (50 mL) was added triethylamine (1.01 g, 10 mmol), followed by an addition of chloropropionyl chloride (2.54 g, 20 mmol). The reaction mixture was stirred at room temperature for 4 hours. The resulting reaction mixture was partitioned between dichloromethane and saturated NaHCO₃ aqueous solution. The organic layer was washed with saturated NaCl aqueous solution, dried over MgSO₄, filtered, and concentrated. The residue was stirred with dichloromethane (20 mL) and suction filtered. The solid was dried further in vacuo to give 3-chloro-(4-nitrophenyl)propanamide (1.85 g, 8.09 mmol, 81%) as a yellow solid. Used directly in the next step without further purification.

### 3-(Dimethylamino)-N-(4-nitrophenyl)propanamide

Into a solution of 3-chloro-(4-nitrophenyl)propanamide (228.6 mg, 1.0 mmol) in methanol (20 ml) was added a 2M solution of dimethylamine in THF (5 mL, 10 mmol). The reaction mixture was stirred at room temperature for 14 hours. The resulting reaction mixture was concentrated and partitioned between ethyl acetate and saturated NaHCO₃ aqueous solution. The organic layer was washed with saturated NaCl aqueous solution, dried over MgSO₄, suction filtered, concentrated and dried further in vacuo to give 3-(dimethylamino)-N-(4-nitrophenyl)propanamide (237 mg, 1 mmol, 100%) as a light yellow solid. Used directly in the next step without further purification.

### N-(4-Aminophenyl)-3-(dimethylamino)propanamide

Into a solution of 3-(dimethylamino)-N-(4-nitrophenyl)propanamide (1 g, 4.21 mmol) in anhydrous methanol (40 mL) was added Pd/C (10%, 1 g). A balloon of hydrogen gas (˜2 L) was inserted into the reaction flask. The reaction mixture was stirred under the hydrogen balloon pressure at room temperature for 4 hours. The resulting reaction mixture was suction filtered through a Celite™ bed. The filtrate was concentrated, dried further in vacuo to give N-(4-aminophenyl)-3-(dimethylamino)propanamide (870 mg, 4.2 mmol, 99%) as a light purple solid. Used directly in the next step without further purification.

### 3-(Dimethylamino)-N-{4-[3-(3-oxo-2,3-dihydrobenzofuran-5-yl)ureido]phenyl}propanamide

Into a solution of 5-amino-1-benzofuran-3(2H)-one (149.2 mg, 1.0 mmol) in dichloromethane (30 mL) was added triethylamine (132.5 μL, 1.0 mmol) followed by addition of triphosgene (89 mg, 0.3 mmol). The mixture was stirred at room temperature for 1 hour and N-(4-aminophenyl)-3-(dimethylamino)propanamide (207 mg, 1.0 mmol) was added. The reaction was stirred at room temperature for 2 hours. The resulting reaction mixture was suction filtered. The solid was dried further in vacuo to give 3-(dimethylamino)-N-{4-[3-(3-oxo-2,3-dihydrobenzofuran-5-yl)ureido]phenyl}propanamide (320 mg). Used directly in the next step without further purification.

### N-[4-({[(2Z)-2-({5-Methoxy-2-methyl-1-[2-(4-methylpiperazin-1-yl)ethyl]-1H-indol-3-yl}methylidene)-3-oxo-2,3-dihydro-1-benzofuran-5-yl]carbamoyl}amino)phenyl]-N3, N3-dimethyl-beta-alaninamide MS (m/z): 608.3 (MH⁺)

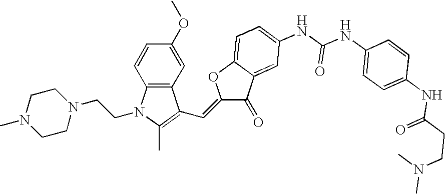

### N-{4-[({(2Z)-2-[(1-Ethyl-5-methoxy-2-methyl-1H-indol-3-yl)methylidene]-3-oxo-2,3-dihydro-1-benzofuran-5-yl}carbamoyl)amino]phenyl}-N3,N3-dimethyl-beta-alaninamide MS (m/z): 582.3 (MH⁺)

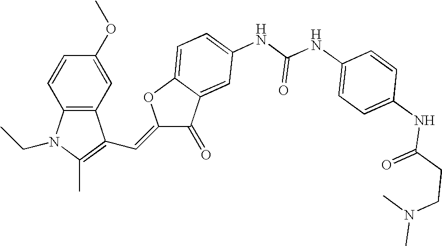

### N-{4-[({(2Z)-2-[(1-Ethyl-5-methoxy-2-methyl-1H-indol-3-yl)methylidene]-3-oxo-2,3-dihydro-1-benzofuran-5-yl}carbamoyl)amino]phenyl}-N,N3,N3-trimethyl-beta-alaninamide MS (m/z): 596.2 (MH⁺)

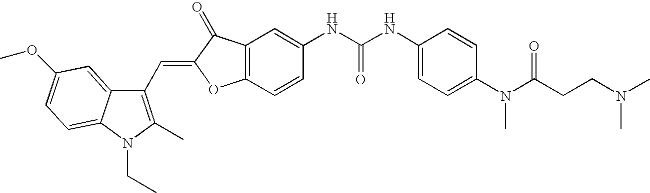

### N-[4-({[(2Z)-2-({5-Methoxy-2-methyl-1-[2-(4-methylpiperazin-1-yl)ethyl]-1H-indol-3-yl}methylidene)-3-oxo-2,3-dihydro-1-benzofuran-5-yl]carbamoyl}amino)phenyl]-N,N3, N3-trimethyl-beta-alaninamide MS (m/z): 347.7 [M+2H]

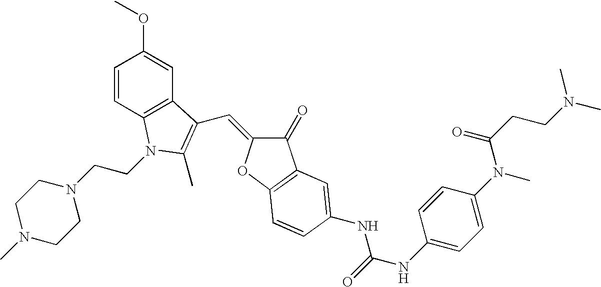

### N-(4-{[(2-{[5-Methoxy-2-(1,3,5-trimethyl-1H-pyrazol-4-yl)-1H-indol-3-yl]methylidene}-3-oxo-2,3-dihydro-1-benzofuran-5-yl)carbamoyl]amino}phenyl)-N,N3,N3-trimethyl-beta-alaninamide MS (m/z): 662.4 (MH⁺)

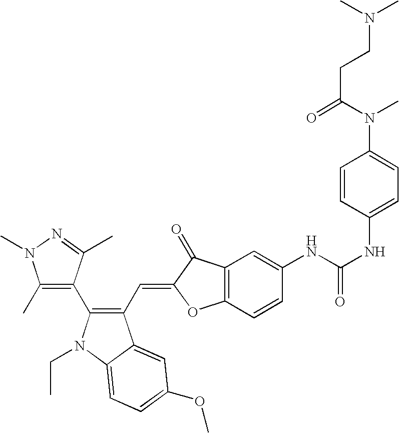

### 1-[(2Z)-2-({5-Methoxy-2-methyl-1-[2-(4-methylpiperazin-1-yl)ethyl]-1H-indol-3-yl}methylene)-6-methyl-3-oxo-2,3-dihydro-1-benzofuran-5-yl]-3-methylurea MS (m/z): 518.3 (MH⁺)

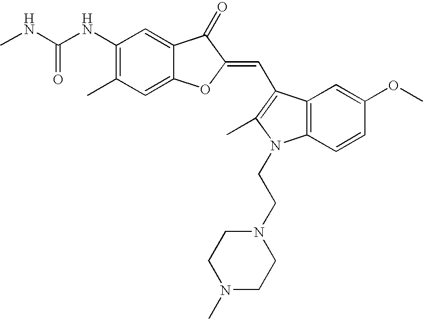

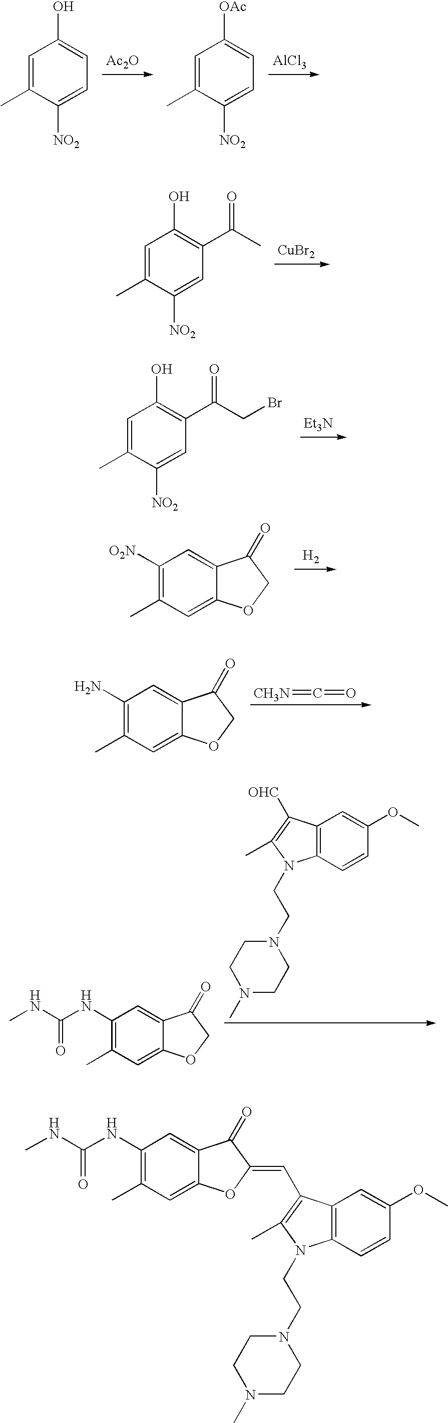

### Step A. 3-Methyl-4-nitrophenyl acetate

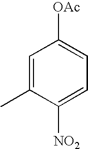

A mixture of 3-methyl-4-nitrophenol (7.7 g, 50 mmol), lithium perchlorate (500 mg), and magnesium sulfate (500 mg) in 50 mL of acetic anhydride was stirred at 80° C. for 30 minutes and concentrated. The residue was partitioned between ethyl acetate and water. The organic layer was dried over magnesium sulfate and filtered through a short pad of silica gel to give 3-methyl-4-nitrophenyl acetate as brown oil. Yield: 94%. MS (m/z): 195.1 (M).

### Step B. 1-(2-Hydroxy-4-methyl-5-nitrophenyl)ethanone

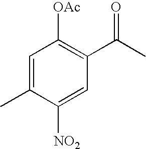

To a mixture of aluminum chloride (1.48 g, 11 mmol) in 12 mL of nitrobenzene was added 3-methyl-4-nitrophenyl acetate (2.15 g, 11 mmol) slowly. The mixture was stirred at 140° C. for 6 hours, and poured into a mixture of 100 g of ice and 60 mL of concentrated HCl. The product was extracted with ethyl acetate and the organic layer was washed with 10% NaOH solution. The alkali solution was neutralized with concentrated HCl, and the product was extracted with ethyl acetate. The organic layer is dried over magnesium sulfate and concentrated. The residue was chromatographed over silica gel, eluting with a gradient of hexanes to 10% ethyl acetate in hexanes to give 1-(2-hydroxy-4-methyl-5-nitrophenyl)ethanone as off-white needles. Yield: 12%. MS (m/z): 194.1 (MH−).

The remaining steps follow the procedure described earlier

### 1-[(2Z)-2-({5-methoxy-2-methyl-1-[2-(4-methylpiperazin-1-yl)ethyl]-1H-indol-3-yl}methylidene)-7-methyl-3-oxo-2,3-dihydro-1-benzofuran-5-yl]-3-methylurea MS (m/z): 518.3 (MH⁺)

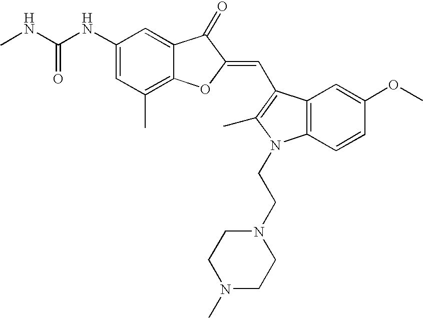

Prepared in the same manner as the previous example, starting from 2-methyl-4-nitrophenol.

### 1-{(2Z)-2-[(1-Ethyl-5-methoxy-2-methyl-1H-indol-3-yl)methylene]-3-oxo-2,3-dihydro-1-benzofuran-5-yl}-3-{4-[(1-methylpiperidin-4-yl)carbonyl]phenyl}urea MS (m/z): 593.3 (MH⁺)

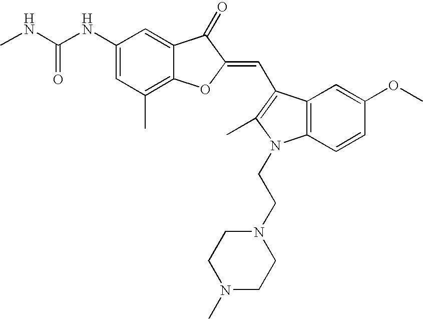

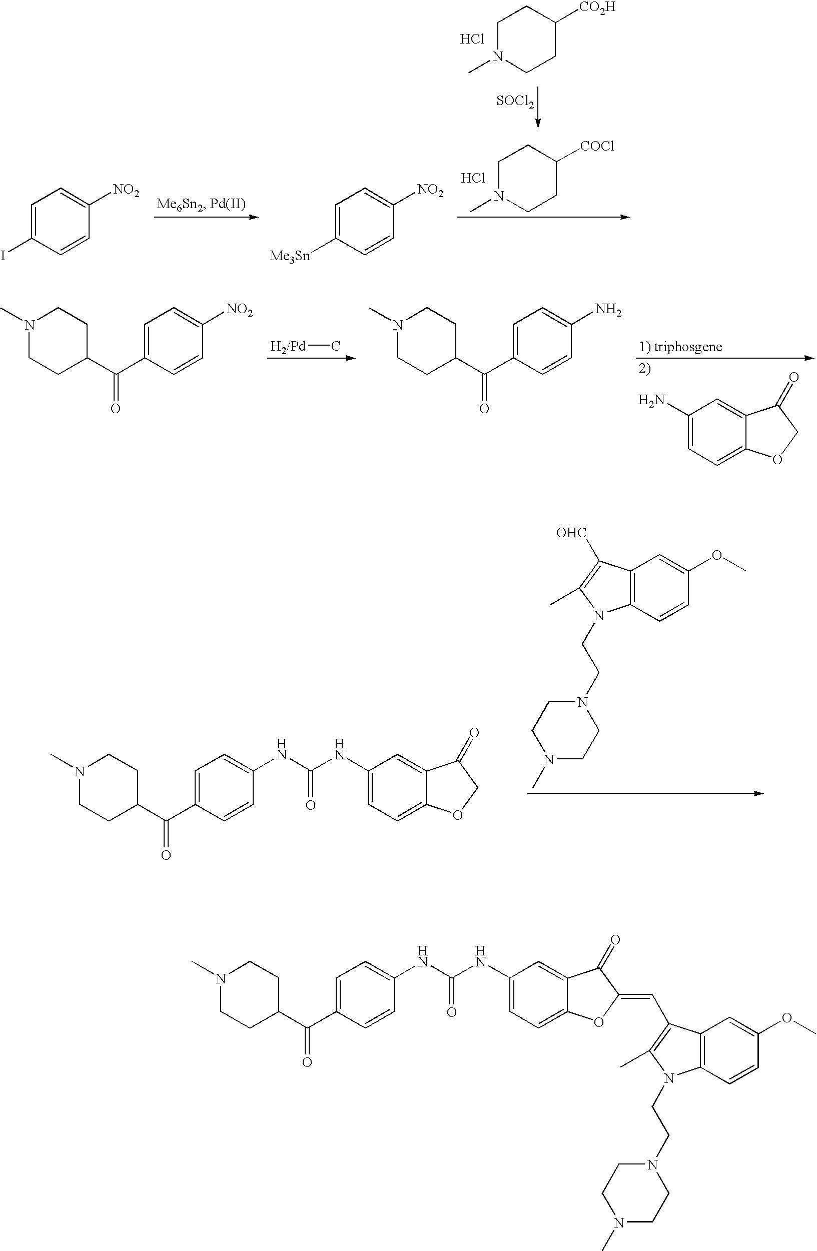

### Step A. (1-Methylpiperidin-4-yl)(4-nitrophenyl)methanone

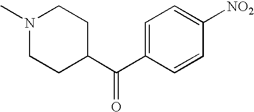

A mixture of 1-methylpiperidine carboxylic acid hydrochloride (1.8 g, 10 mmol) and 20 mL of thionyl chloride was stirred at reflux for 1 hour and concentrated. The crude product was used directly in the next step.

A mixture of 1-iodo-4-nitrobenzene (600 mg, 2.4 mmol), hexamethylditin (1.0 g, 3 mmol), and pi-allyl palladium dichloride dimmer (10 mg) in 10 mL of DMF was stirred at room temperature for 2 hours. 1-Methylpiperidine-4-carbonyl chloride hydrochloride (1.0 g, 5 mmol, from previous step) was added and the mixture was stirred at room temperature for 18 hours. The reaction mixture was partitioned between ethyl acetate and water. The organic layer was washed with water (×2) and brine (×2), dried over magnesium sulfate, and concentrated. The residue is chromatographed over silica gel, eluting with a gradient of ethyl acetate to 50% methanol in ethyl acetate to give (1-methylpiperidin-4-yl)(4-nitrophenyl)methanone as a yellow solid. Yield: 41%. MS (m/z): 249.1 (MH⁺).

The remaining steps follow the procedure described earlier.

### N-[2-(Dimethylamino)ethyl]-4-[({(2Z)-2-[(1-ethyl-5-methoxy-2-methyl-1H-indol-3-yl)methylene]-3-oxo-2,3-dihydro-1-benzofuran-5-yl}carbamoyl)amino]-N-methylbenzamide MS (m/z): 596.2 (MH⁺)

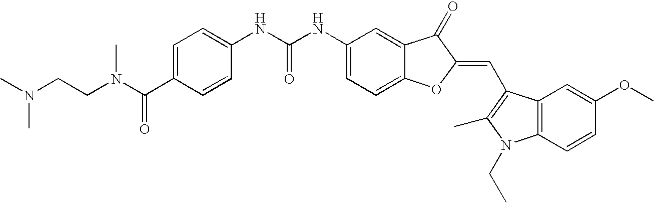

### 1-(4-{[4-(Dimethylamino)piperidin-1-yl]carbonyl}phenyl)-3-{(2Z)-2-[(1-ethyl-5-methoxy-2-methyl-1H-indol-3-yl)methylene]-3-oxo-2,3-dihydro-1-benzofuran-5-yl}urea MS (m/z): 622.3 (MH⁺)

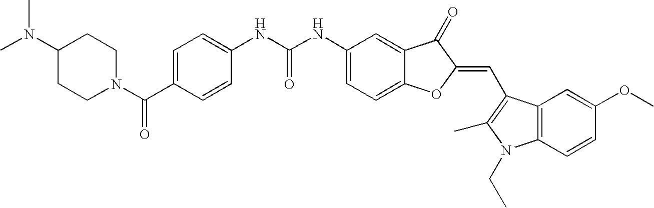

### 1-(4-{[3-(Dimethylamino)propyl](methyl)amino}phenyl)-3-{(2Z)-2-[(1-ethyl-5-methoxy-2-methyl-1H-indol-3-yl)methylene]-3-oxo-2,3-dihydro-1-benzofuran-5-yl}urea MS (m/z): 582.3 (MH⁺)

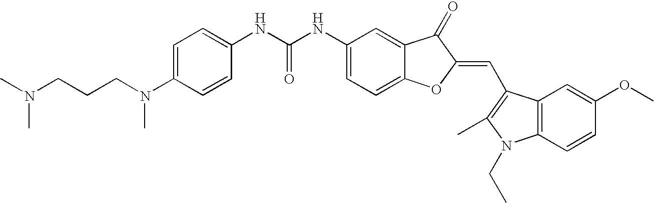

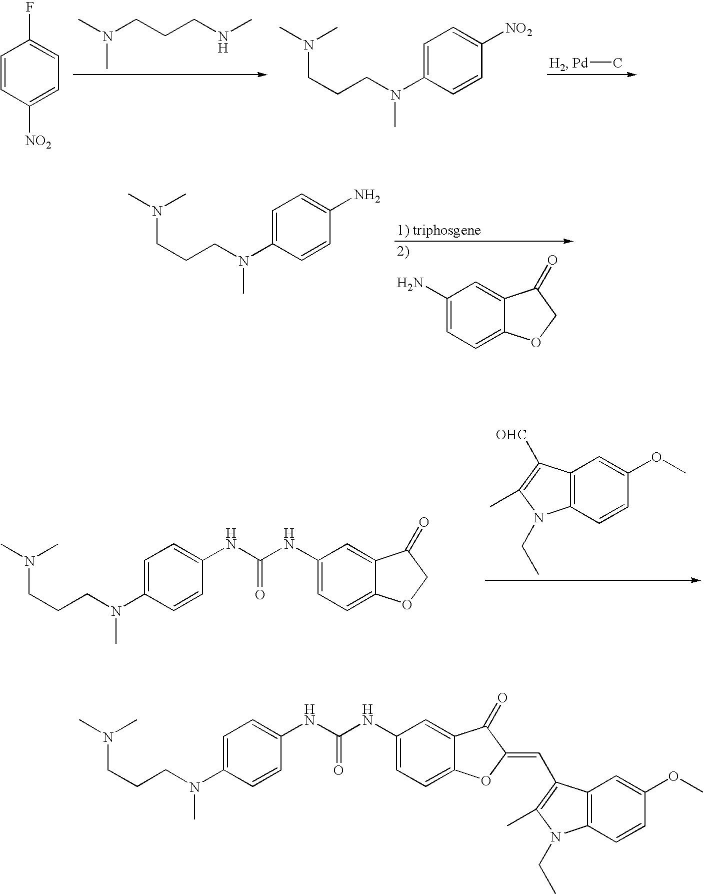

### Step A. NNN Trimethyl-N′-(4-nitrophenyl)propane-1,3-diamine

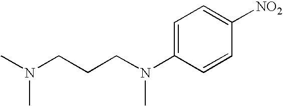

A mixture of 1-fluoro-4-nitrobenzene (705 mg, 5 mmol), N,N,N′-trimethyl-1,3-propanediamine (1 mL, excess) and 1.0 g of potassium carbonate in 50 mL of DMF was stirred at 60° C. for 2 h and concentrated. The residue was chromatographed over silica gel, eluting with a gradient of ethyl acetate to 50% methanol in ethyl acetate to N,N,N′-trimethyl-N′-(4-nitrophenyl)propane-1,3-diamine as a yellow oil. The product was used directly in the next step.

The remaining steps follow the procedure described earlier.

### 1-{4-[3-(Dimethylamino)propoxy]phenyl}-3-{(2Z)-2-[(1-ethyl-5-methoxy-2-methyl-1H-indol-3-yl)methylene]-3-oxo-2,3-dihydro-1-benzofuran-5-yl}urea MS (m/z): 569.3 (MH⁺)

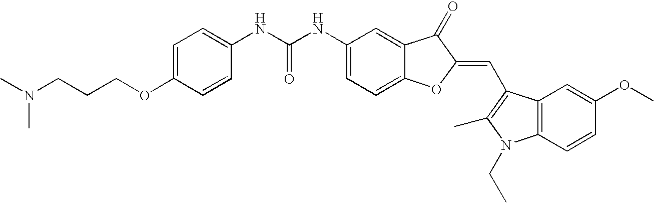

### 1-(4-{[2-(Dimethylamino)ethyl](methyl)amino}phenyl)-3-{(2Z)-2-[(1-ethyl-5-methoxy-2-methyl-1H-indol-3-yl)methylene]-3-oxo-2,3-dihydro-1-benzofuran-5-yl}urea MS (m/z): 568.3 (MH⁺)

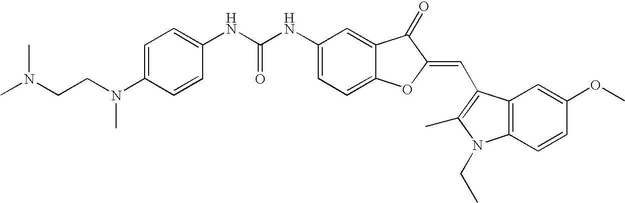

### 1-{4-[2-(dimethylamino)ethoxy]phenyl}-3-{(2Z)-2-[(1-ethyl-5-methoxy-2-methyl-1H-indol-3-yl)methylidene]-3-oxo-2,3-dihydro-1-benzofuran-5-yl}urea MS (m/z): 555.2 (MH⁺)

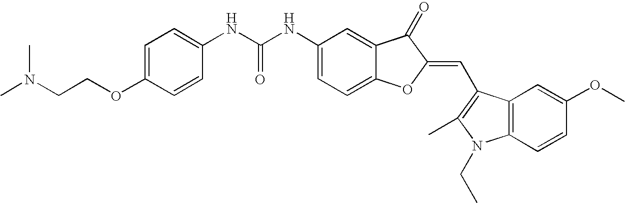

### 1-{4-[4-(Dimethylamino)butoxy]phenyl}-3-{(2Z)-2-[(1-ethyl-5-methoxy-2-methyl-1H-indol-3-yl)methylidene]-3-oxo-2,3-dihydro-1-benzofuran-5-yl}urea MS (m/z): 583.3 (MH⁺)

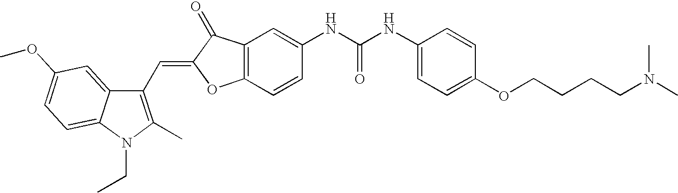

### 1-(4-{[4-(Dimethylamino)butyl](methyl)amino}phenyl)-3-{(2Z)-2-[(1-ethyl-5-methoxy-2-methyl-1H-indol-3-yl)methylidene]-3-oxo-2,3-dihydro-1-benzofuran-5-yl}urea MS (m/z): 596.3 (MH⁺)

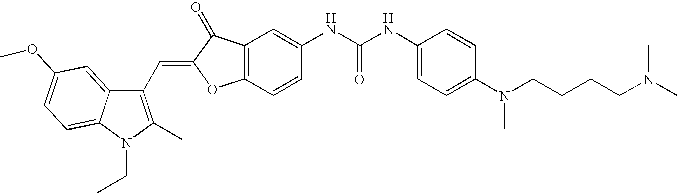

### 1-{4-[4-(Dimethylamino)butoxy]phenyl}-3-[(2Z)-2-({5-methoxy-2-methyl-1-[2-(4-methylpiperazin-1-yl)ethyl]-1H-indol-3-yl}methylidene)-3-oxo-2,3-dihydro-1-benzofuran-5-yl]urea MS (m/z): 341.2 (M2H⁺⁺)

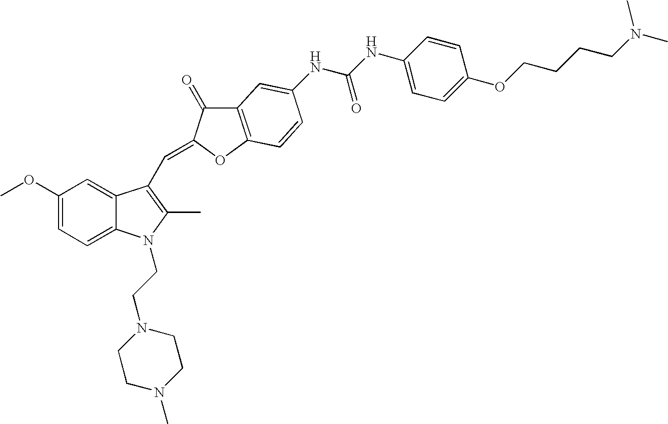

### 1-(4-{[2-(Dimethylamino)ethyl]amino}phenyl)-3-{(2Z)-2-[(1-ethyl-5-methoxy-2-methyl-1H-indol-3-yl)methylidene]-3-oxo-2,3-dihydro-1-benzofuran-5-yl}urea MS (m/z): 554.3 (MH⁺)

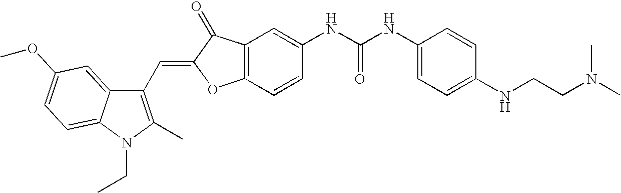

### 1-(4-{[2-(4-Dimethylamino)ethyl]amino}phenyl)-3-[(2Z)-2-({5-methoxy-2-methyl-1-[2-(4-methylpiperazin-1-yl)ethyl]-1H-indol-3-yl}methylidene)-3-oxo-2,3-dihydro-1-benzofuran-5-yl]urea MS (m/z): 652.4 (MH⁺)

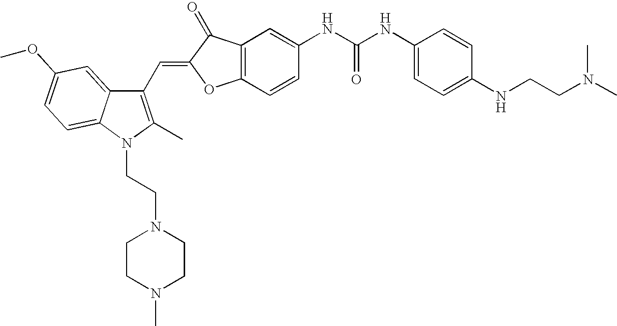

### 1-{(2Z)-2-[(1-Ethyl-5-methoxy-2-methyl-1H-indol-3-yl)methylidene]-3-oxo-2,3-dihydro-1-benzofuran-5-yl}-3-{4-[3-(methylamino)propoxy]phenyl}urea MS (m/z): 555.3 (MH⁺)

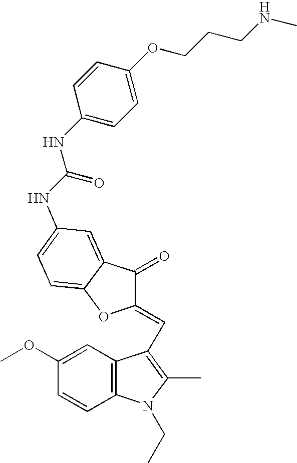

### 1-{4-[4-(Dimethylamino)butyl]phenyl}-3-{(2Z)-2-[(1-ethyl-5-methoxy-2-methyl-1H-indol-3-yl)methylidene]-3-oxo-2,3-dihydro-1-benzofuran-5-yl}urea MS (m/z): 567.3 (MH⁺)

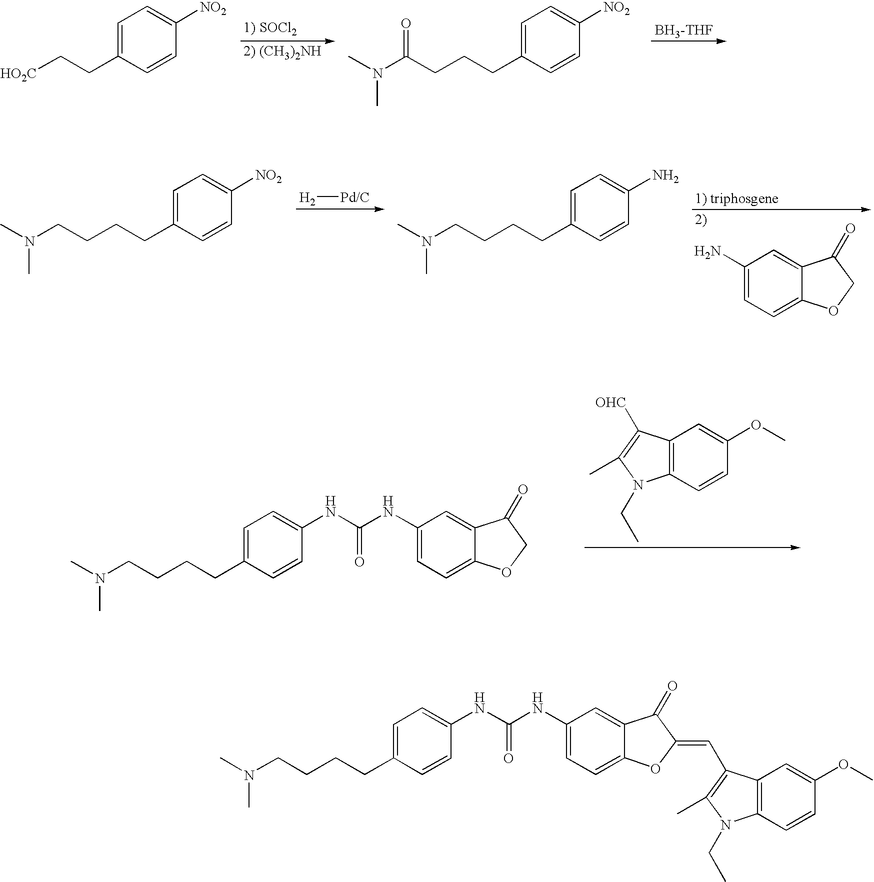

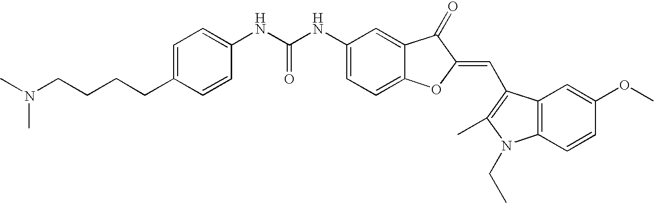

### Step A. N,N-Dimethyl-4-(4-nitrophenyl)butanamide

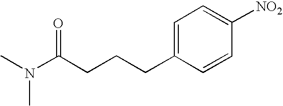

A mixture of 4-(p-nitrophenyl)butyric acid (1.05 g, 5.0 mmol) and 10 mL of thionyl chloride was stirred under reflux for 1 hour and concentrated. The residue was dissolved in 20 mL of THF and dimethyl amine (2 N in THF, 10 mL, 20 mmol) was added. The mixture was stirred at room temperature for 30 minutes, concentrated, and partitioned between ethyl acetate and water. The organic layer was washed with saturated sodium chloride solution, dried over magnesium sulfate, and filtered through a short pad of silica gel to give N,N-dimethyl-4-(4-nitrophenyl)butanamide as a light yellow solid. Yield: 77%.

### Step B. N,N-Dimethyl-4-(4-nitrophenyl)butan-1-amine

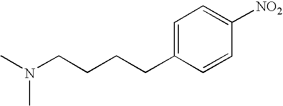

To 25 mL of borane-tetrahydrofuran complex (1.0 M in THF, 25 mmol) at room temperature was added N,N-dimethyl-4-(4-nitrophenyl)butanamide (910 g, 3.85 mmol). The mixture was stirred under reflux for 2 hours, and cooled to 0° C. HCl (2.0 N, 10 mL, 20 mmol) was added, and the mixture was concentrated. To this residue was added conc. HCl (10 mL), and the mixture was reflux for 1 hour and cooled to room temperature. The solution was made alkaline by adding sodium hydroxide, and the product was extracted with ethyl acetate. The organic layer was extracted with 1N HCl, and the aqueous layer was made alkaline by adding sodium hydroxide. The product was extracted with ethyl acetate. The organic layer was washed with 10% NaOH solution. The alkali solution was neutralized with concentrated HCl, and the product was extracted with ethyl acetate. The organic layer was washed with saturated sodium chloride solution, dried over magnesium sulfate, and concentrated to give N,N-dimethyl-4-(4-nitrophenyl)butan-1-amine as a yellow oil. Yield: 56%.

The remaining steps follow the procedure described earlier.

### 1-{4-[3-(Dimethylamino)propyl]phenyl}-3-{(2Z)-2-[(1-ethyl-5-methoxy-2-methyl-1H-indol-3-yl)methylidene]-3-oxo-2,3-dihydro-1-benzofuran-5-yl}urea MS (m/z): 553.2 (MH⁺)

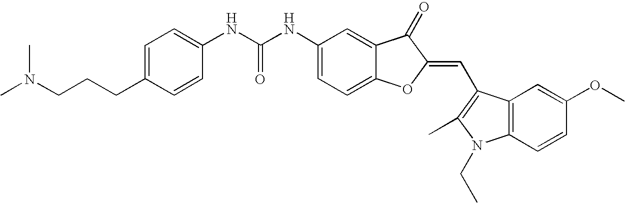

### 1-{4-[2-(Dimethylamino)ethyl]phenyl}-3-{(2Z)-2-[(1-ethyl-5-methoxy-2-methyl-1H-indol-3-yl)methylidene]-3-oxo-2,3-dihydro-1-benzofuran-5-yl}urea MS (m/z): 539.3 (MH⁺)

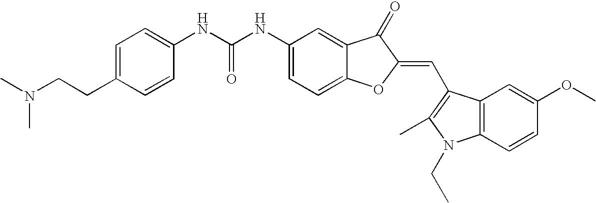

### 1-{4-[(Dimethylamino)methyl]phenyl}-3-{(2Z)-2-[(1-ethyl-5-methoxy-2-methyl-1H-indol-3-yl)methylidene]-3-oxo-2,3-dihydro-1-benzofuran-5-yl}urea MS (m/z): 525.2 (MH⁺)

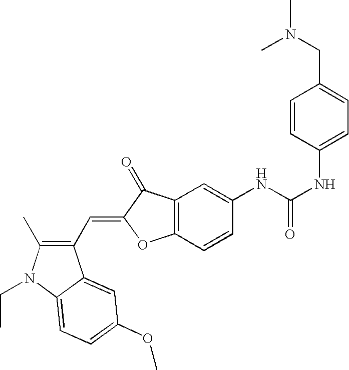

To a stirred solution of triphosgene (31.8 mg, 0.107 mmol) in anhydrous tetrahydrofuran (1 mL) was added 5-aminobenzofuran-3(2H)-one (26.6 mg, 0.179 mmol) at 25° C. The reaction mixture was stirred for 15 minutes and TEA (25 mL, 0.18 mmol, 1 eq) was added and the stirring was continued for an additional 1 hr. Then a mixture of 4-[(dimethylamino)methyl]aniline, HCl (100 mg, 0.536 mmol), TEA (25 mL, 0.18 mmol, 1 eq) in THF (1 mL) was added and stirred for another 2 hours. TEA (406 μL, 2.91 mmol) was added and the mixture was stirred over night. The solvents were removed in a N₂ stream and the crude mixture was purified by semi-prep-HPLC (NH3-method) to give the desired product as off-white solid. LC/MS didn't show M⁺ only M⁺-NMe2, but ¹H-NMR was consistent).

### 1-[4-(Dimethylamino)phenyl]-3-{(2Z)-2-[(1-ethyl-5-methoxy-2-methyl-1H-indol-3-yl)methylidene]-3-oxo-2,3-dihydro-1-benzofuran-5-yl}urea MS (m/z): 511.2 (MH⁺)

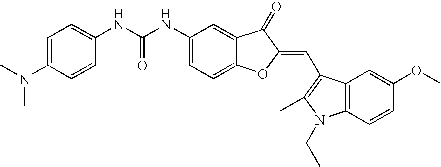

### (Z)-1-(2-((2-(3,5-dimethylisoxazol-4-yl)-5-methoxy-1-(2-(4-methylpiperazin-1-yl)ethyl)-1H-indol-3-yl)methylene)-3-oxo-2,3-dihydrobenzofuran-5-yl)-3-methylurea MS (m/z): 585.3 (MH⁺)

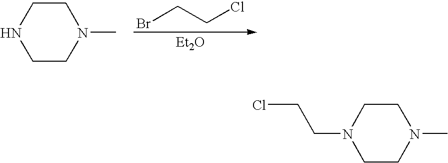

### Preparation of 1-(2-chloroethyl)-4-methylpiperazine

1-Methylpiperazine (22 mL, 200 mmol) added to stirred 1-bromo-2-chloroethane (17 mL, 200 mmol) in Et₂O (200 mL) at 0° C. over 5 minutes. Resulting mixture warmed to room temperature and stirred for 3 days. The mixture filtered and solvent removed from filtrate. Residue from filtrate dissolved in 1:1 THF-hexanes (150 mL) and resulting solution stirred at 45° C. for 2 days. The mixture filtered and filtrate concentrated at 45° C. Yield: 30%. Material used without purification.

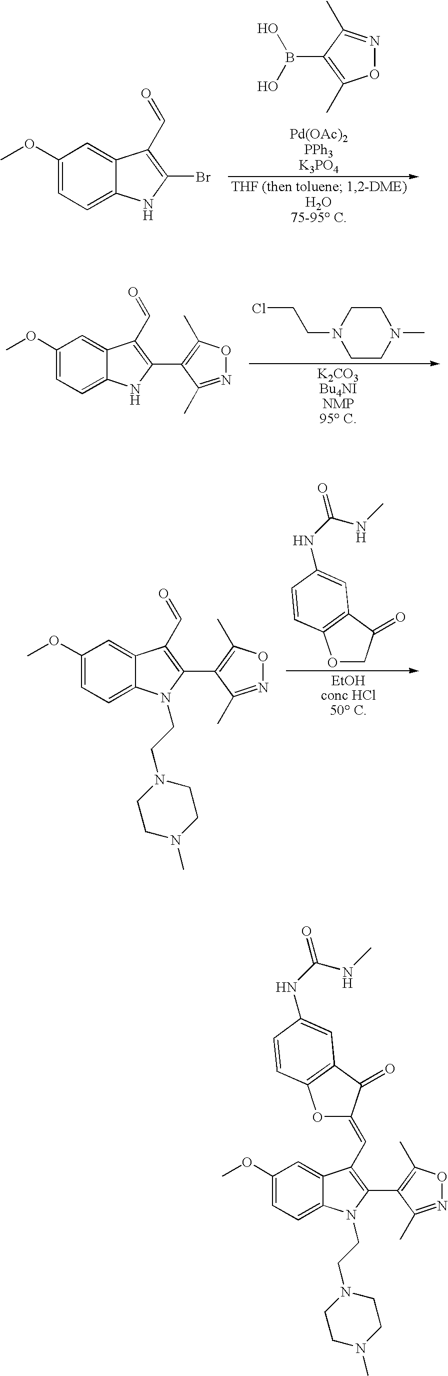

### Preparation of 2-(3,5-dimethylisoxazol-4-yl)-5-methoxy-1H-indole-3-carbaldehyde

A mixture of 2-bromo-5-methoxy-1H-indole-3-carbaldehyde (300 mg, 1.18 mmol), 3,5-dimethylisoxazole-4-boronic acid (333 mg, 2.36 mmol), Pd(OAc)₂ (13 mg, 0.06 mmol), PPh₃ (63 mg, 0.24 mmol), and K₃PO₄ (751 mg, 3.54 mmol) in THF (2.3 mL), and water (2 mL) was stirred under N₂ in a sealed vial at 75° C. overnight. THF was replaced by 1,2-dimethoxyethane (2 mL) and toluene (1 mL) and resulting mixture stirred at 95° C. for 5 hours, then cooled to room temperature. Water (3 mL) added to the mixture and then extracted with EtOAc (3×10 mL). Extracts combined, dried over Na₂SO₄ and concentrated. The mixture purified by silica gel column chromatography (eluent: 45% EtOAc-hexanes). Yield: 79%. MS (m/z): 269.1 (MH−).

### Preparation of 2-(3,5-dimethylisoxazol-4-yl)-5-methoxy-1-(2-(4-methylpiperazin-1-yl)ethyl)-1H-indole-3-carbaldehyde

A mixture of 2-(3,5-dimethylisoxazol-4-yl)-5-methoxy-1H-indole-3-carbaldehyde (102 mg, 0.38 mmol), 1-(2-chloroethyl)-4-methylpiperazine (124 mg, 0.76 mmol), K₂CO₃ (146 mg, 1.06 mmol), and a catalytic amount of Bu₄NI in NMP (0.8 mL) was stirred at 80° C. overnight, then 95° C. over an additional 24 hours. Reaction mixture cooled to room temperature, diluted with EtOAc and extracted using 0.5 M aqueous HCl. Aqueous layer was made basic using saturated aqueous Na₂CO₃ then extracted with EtOAc. Organic layer collected and concentrated. The mixture purified by silica gel column chromatography (eluent: 94:3:3 EtOAc-MeOH-Et₃N). Yield: 27%. MS (m/z): 397.2 (MH⁺).

### (Z)-N-(1-Hydroxy-2-methylpropan-2-yl)-5-methoxy-3-((5-(3-methylureido)-3-oxobenzofuran-2(3H)-ylidene)methyl)-1H-indole-2-carboxamide

condensation procedure MS (m/z): 479.2 (MH⁺).

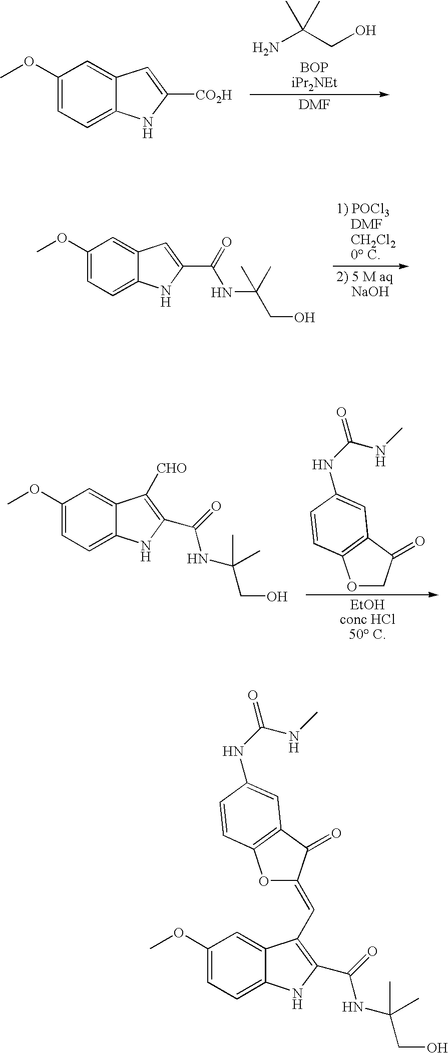

### Preparation of N-(1-hydroxy-2-methylpropan-2-yl)-5-methoxy-1H-indole-2-carboxamide

(Benzotriazol-1-yloxy)tris(dimethylamino)phosphonium hexafluorophosphate (1.97 g, 4.5 mmol) added to a stirred mixture of 5-methoxyindole-2-carboxylic acid (810 mg, 4.2 mmol) and iPr₂NEt (770 μL, 4.7 mmol) in DMF (10 mL) at room temperature. Resulting mixture stirred for 5 minutes then 2-amino-2-methyl-1-propanol (488 μL, 5.1 mmol) added. The mixture stirred overnight then poured into 0.5 M aqueous HCl (25 mL) and extracted with EtOAc (3×50 mL). EtOAc extracts combined, washed with saturated aqueous NaHCO₃ (2×50 mL), water (2×25 mL), and then aqueous NaCl (25 mL). EtOAc extract dried over Na₂SO₄ and concentrated. Resulting tan solid rinsed with EtOAc (2×10 mL) and dried in vacuo. Yield: 65%. MS (m/z): 263.2 (MH⁺).

### Preparation of 3-formyl-N-(1-hydroxy-2-methylpropan-2-yl)-5-methoxy-1H-indole-2-carboxamide

DMF (156 μL, 2.0 mmol) was added to a stirred solution of phosphorus oxychloride (190 μL, 2.0 mmol) in CH₂Cl₂ (0.5 mL) at 0° C. Resulting mixture stirred for 15 minutes then a mixture of N-(1-hydroxy-2-methylpropan-2-yl)-5-methoxy-1H-indole-2-carboxamide (134 mg, 0.51 mmol) in CH₂Cl₂ (2.5 mL) added and the resulting mixture stirred at room temperature for 1 hours. The mixture cooled to 0° C., then 5 M aqueous NaOH added (5 mL) and the mixture stirred for 15 minutes at room temperature. The mixture diluted with water (25 mL) and extracted with CH₂Cl₂ (3×40 mL). CH₂Cl₂ extracts combined, washed with saturated aqueous NaCl (25 mL), dried over Na₂SO₄, and concentrated. Crude mixture purified by prep HPLC. Yield: 22%. MS (m/z): 291.1 (MH⁺).

### (Z)-1-(2-((2-(3-Cyclopropyl-1,2,4-oxadiazol-5-yl)-5-methoxy-1-(2-(4-methylpiperazin-1-yl)ethyl)-1H-indol-3-yl)methylene)-3-oxo-2,3-dihydrobenzofuran-5-yl)-3-methyl urea MS (m/z): 598.3 (MH⁺)

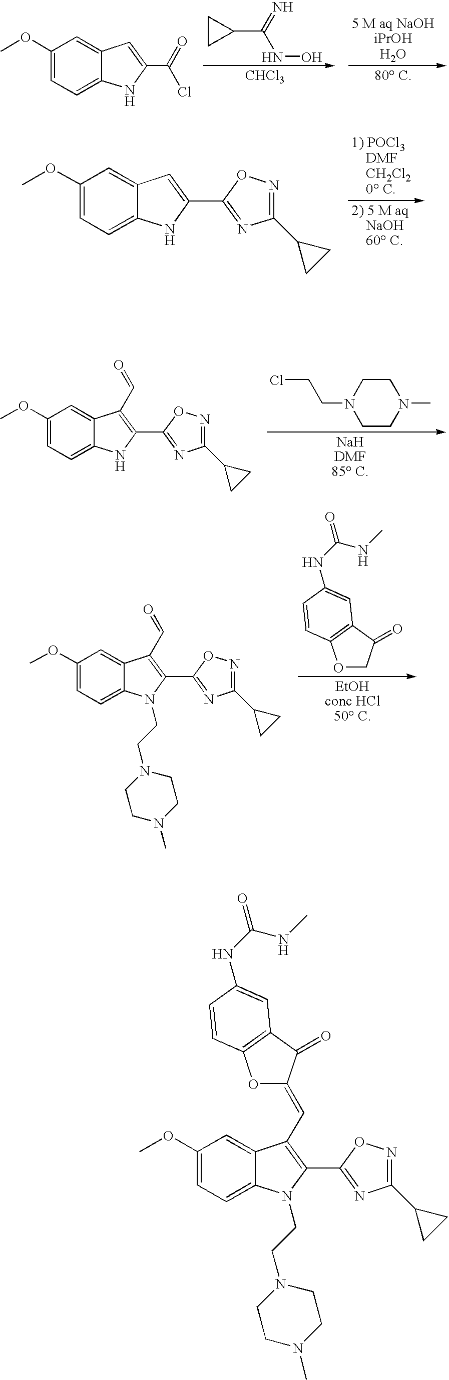

### Preparation of 3-cyclopropyl-5-(5-methoxy-1H-indol-2-yl)-1,2,4-oxadiazole

A mixture of 5-methoxy-1H-indole-2-carbonyl chloride (384 mg, 1.83 mmol) and N′-hydroxycyclopropanecarboximidamide (200 mg, 2.00 mmol) in chloroform (5 mL) was stirred at reflux for 30 minutes then cooled to room temperature and concentrated. The residue treated with isopropyl alcohol (10 mL), water (10 mL), and 5 M aqueous NaOH (5 mL) and the resulting mixture stirred at 80° C. for 45 minutes. Reaction mixture cooled to room temperature, poured into water (50 mL), and extracted with EtOAc (3×50 mL). EtOAc extracts combined, washed with saturated aqueous NaCl (25 mL), dried over Na₂SO₄, and concentrated. The mixture purified by silica gel column chromatography (eluent: 15% EtOAc-hexanes). Yield: 38%. MS (m/z): 256.1 (MH⁺).

### Preparation of 2-(3-cyclopropyl-1,2,4-oxadiazol-5-yl)-5-methoxy-1H-indole-3-carbaldehyde

DMF (241 μL, 3.10 mmol) added to stirred POCl₃ (289 μL, 3.10 mmol) at 0° C. and resulting mixture stirred for 2 minutes then diluted with CH₂Cl₂ (0.5 mL). Resulting mixture stirred for 15 minutes then a solution of 3-cyclopropyl-5-(5-methoxy-1H-indol-2-yl)-1,2,4-oxadiazole (159 mg, 0.62 mmol) in CH₂Cl₂ (2 mL) added and mixture stirred for 1 hours. Reaction mixture treated with water (1 mL), then slowly with 5 M aqueous NaOH (3 mL). Resulting mixture stirred at 60° C. for 5 minutes, cooled to room temperature, diluted with water (25 mL), and extracted with EtOAc (2×50 mL). EtOAc extracts combined, washed with saturated aqueous NaCl (25 mL), dried over Na₂SO₄, and concentrated. Yield: 89%. MS (m/z): 284.1 (MH⁺).

### Preparation of 2-(3-cyclopropyl-1,2,4-oxadiazol-5-yl)-5-methoxy-1-(2-(4-methylpiperazin-1-yl)ethyl)-1H-indole-3-carbaldehyde

A solution of 2-(3-cyclopropyl-1,2,4-oxadiazol-5-yl)-5-methoxy-1H-indole-3-carbaldehyde (32 mg, 0.11 mmol) in DMF (0.5 mL) was added slowly to NaH (excess) and resulting mixture stirred for 5 minutes. 1-(2-Chloroethyl)-4-methylpiperazine (23 mg, 0.14 mmol) was added and the resulting mixture stirred at 85° C. overnight. Additional 1-(2-chloroethyl)-4-methylpiperazine (50 mg, 0.28 mmol) added and mixture stirred for another 24 hours, at 85° C. Reaction mixture cooled to room temperature, diluted with EtOAc and extracted using 0.5 M aqueous HCl. Aqueous layer was made basic using saturated aqueous Na₂CO₃ then extracted with EtOAc. Organic layer collected and concentrated. Yield: 40%. MS (m/z): 410.2 (MH⁺).

### (Z)-1-(2-((2-(3-Isopropyl-1,2,4-oxadiazol-5-yl)-5-methoxy-1-(2-(4-methylpiperazin-1-yl)ethyl)-1H-indol-3-yl)methylene)-3-oxo-2,3-dihydrobenzofuran-5-yl)-3-methylurea MS (m/z): 600.3 (MH⁺)

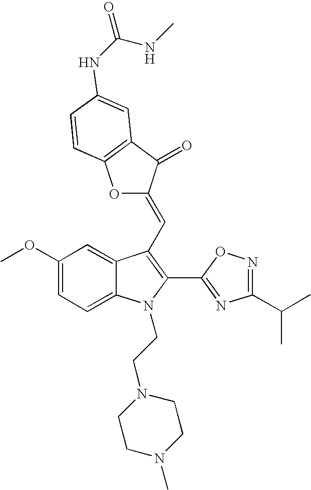

### (Z)-1-(2-((2-tert-Butyl-5-methoxy-1H-indol-3-yl)methylene)-3-oxo-2,3-dihydrobenzofuran-5-yl)-3-methylurea MS (m/z): 420.2 (MH⁺)

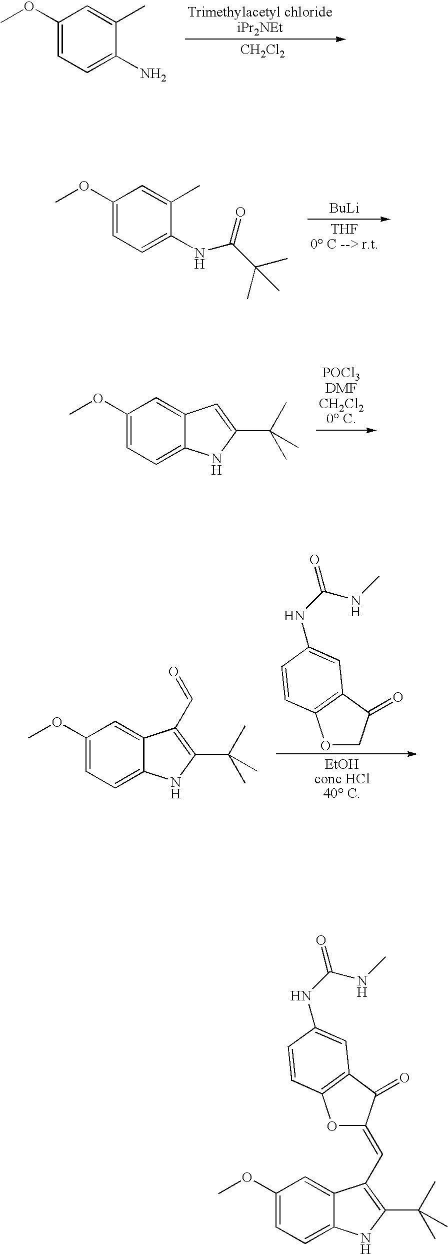

### Preparation of N-(4-methoxy-2-methylphenyl)pivalamide

Trimethylacetyl chloride (2.9 mL, 24 mmol) was added in drops to a stirred solution of 4-methoxy-2-methylaniline (3.1 g, 23 mmol) and iPr₂NEt (4.2 mL, 24 mmol) in CH₂Cl₂ (50 mL) over a period of 2-3 minutes. Resulting mixture stirred for 90 minutes. Solvent removed in vacuo and crude product partitioned between water (25 mL) and 1:1 EtOAc-hexanes (150 mL). Aqueous layer extracted with 1:1 EtOAc-hexanes (50 mL). Organic extracts combined, washed with water (25 mL), saturated aqueous NH₄Cl (25 mL), and saturated aqueous NaCl (25 mL), dried over Na₂SO₄ and concentrated. Yield: >100%. MS (m/z): 222.2 (MH⁺).

### Preparation of 2-tert-butyl-5-methoxy-1H-indole

A solution of BuLi in hexane (2.0 M, 26 mL, 52 mmol) was added slowly to a stirred solution of N-(4-methoxy-2-methylphenyl)pivalamide (˜23 mmol) in THF (100 mL) at 0° C. over a period of 10 minutes. Resulting mixture stirred overnight allowing to warm to room temperature. Reaction mixture slowly poured into stirred 1 M aqueous HCl at 0° C. (150 mL). The mixture extracted with EtOAc (3×100 mL). EtOAc extracts combined, washed with saturated aqueous NaCl, dried over Na₂SO₄, and concentrated. The mixture purified by silica gel column chromatography (eluent: 15% EtOAc-hexanes). Yield: 83%. MS (m/z): 204.2 (MH⁺).

### Preparation of 2-tert-butyl-5-methoxy-1H-indole-3-carbaldehyde

DMF (243 μL, 3.12 mmol) added to stirred POCl₃ (290 μL, 3.12 mmol) at 0° C. and resulting mixture diluted with CH₂Cl₂ (0.5 mL) and stirred for 20 minutes. A solution of 2-tert-butyl-5-methoxy-1H-indole (158 mg, 0.78 mmol) in CH₂Cl₂ (2.5 mL) added and mixture stirred for 45 minutes. Reaction mixture poured into saturated aqueous Na₂CO₃ (25 mL) and extracted with EtOAc (2×50 mL). EtOAc extracts combined, washed with saturated aqueous NaCl (25 mL), dried over Na₂SO₄, and concentrated. Yield: >100%. MS (m/z): 232.2 (MH⁺).

### (Z)-1-(2-((2-Cyano-5-methoxy-1-(2-(4-methylpiperazin-1-yl)ethyl)-1H-indol-3-yl)methylene)-3-oxo-2,3-dihydrobenzofuran-5-yl)-3-methylurea MS (m/z): 515.2 (MH⁺)

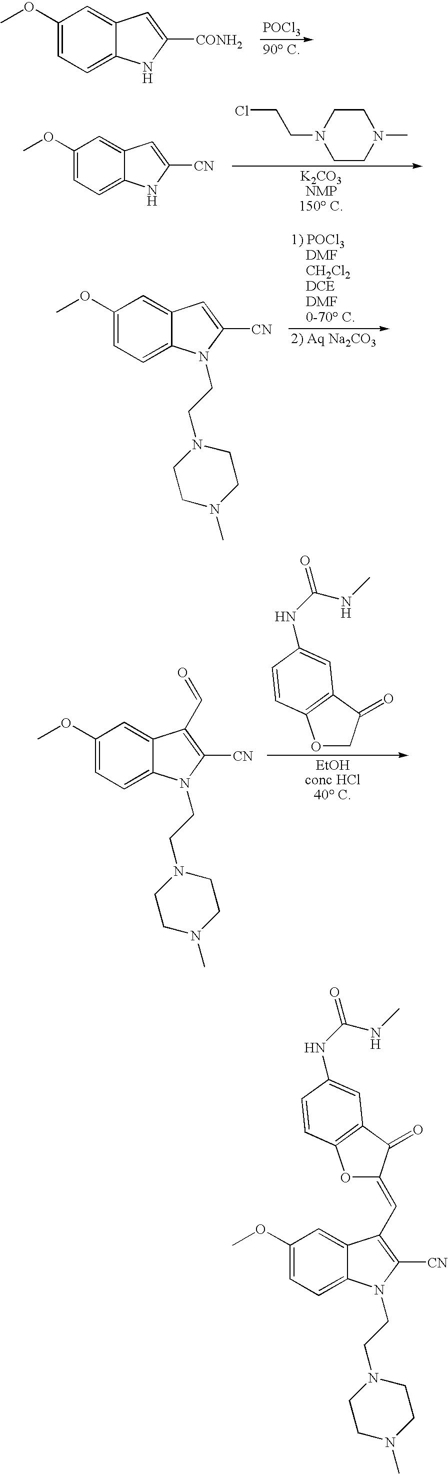

### Preparation of 5-methoxy-1H-indole-2-carbonitrile

Solid 5-methoxy-1H-indole-2-carboxamide (1.59 g, 8.4 mmol) was added to stirred POCl₃ (20 mL) at room temperature. Resulting mixture heated to 90° C., stirred for 45 minutes, and then cooled to room temperature. The mixture poured onto ice (˜100 mL) and let sit for 15 minutes. CH₂Cl₂ (150 mL) added and organic layer washed with 1:1 saturated aqueous Na₂CO₃—H₂O (50 mL), then saturated aqueous NaCl (50 mL), dried over Na₂SO₄, and concentrated. Residue was dried by azeotrope distillation using toluene (2×50 mL) and then dissolved in 60% EtOAc-hexanes and mixture filtered through a plug of SiO₂. Resulting filtrate washed with 1:1 saturated aqueous Na₂CO₃—H₂O until washings remained basic, then washed with saturated aqueous NaCl (50 mL), dried over Na₂SO₄ and concentrated. Yield: 81%. MS (m/z): 171.1 (MH−).

### Preparation of 5-methoxy-1-(2-(4-methylpiperazin-1-yl)ethyl)-1H-indole-2-carbonitrile

A mixture of 5-methoxy-1H-indole-2-carbonitrile (293 mg, 1.70 mmol), K₂CO₃ (1.75 g, 12.7 mmol), and 1-(2-chloroethyl)-4-methylpiperazine (1.41 g, 8.7 mmol) was heated to 150° C. NMP (0.7 mL) was added slowly and the resulting mixture stirred at 150° C. for 2.5 hours. Reaction mixture cooled to room temperature, water added (25 mL) and extracted with EtOAc (2×50 mL). EtOAc extracts combined, washed with water (10 mL), saturated aqueous NaCl (25 mL), dried over Na₂SO₄, and concentrated. The mixture purified by silica gel column chromatography (eluent: 96:2:2 EtOAc-MeOH-Et₃N). Yield: 26%. MS (m/z): 299.2 (MH⁺).

### Preparation of 3-formyl-5-methoxy-1-(2-(4-methylpiperazin-1-yl)ethyl)-1H-indole-2-carbonitrile

DMF (137 μL, 1.76 mmol) added to stirred POCl₃ (164 μL, 1.76 mmol) at 0° C. and resulting mixture diluted with CH₂Cl₂ (0.5 mL) and then stirred for 25 minutes. A solution of 5-methoxy-1-(2-(4-methylpiperazin-1-yl)ethyl)-1H-indole-2-carbonitrile (131 mg, 0.44 mmol) in CH₂Cl₂ (1.5 mL) added and mixture stirred at 50° C. for 3 h. 1,2-Dichloroethane (1 mL) added and mixture stirred at 70° C. for 2 hours. Additional DMF added (0.8 mL) and mixture stirred at 70° C. for 2.5 days. Additional POCl₃ added (0.5 mL) and mixture stirred at 70° C. for an additional 24 hours. Reaction mixture cooled to room temperature, poured slowly into saturated aqueous Na₂CO₃ (25 mL) and extracted with EtOAc (3×40 mL). EtOAc extracts combined, washed with saturated aqueous NaCl (25 mL), dried over Na₂SO₄, and concentrated. The mixture purified by silica gel column chromatography (eluent: 100% EtOAc to 95:2:3 EtOAc-MeOH-Et₃N gradient). Yield 47%. MS (m/z): 327.2 (MH⁺).

### Procedure to make 1-methyl-3-(3-oxo-2,3-dihydro-1-benzofuran-5-yl)urea

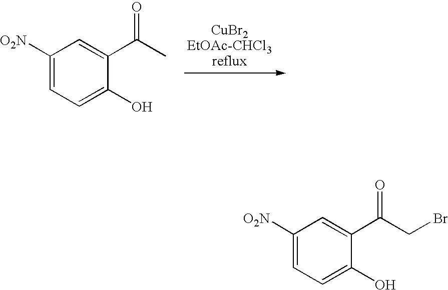

Reference: J. Org. Chem. 1964, 29, 3459

A solution of 2′-hydroxy-5′-nitroacetophenone (5.03 g, 28 mmol) in CHCl₃ (45 mL) was added to a stirred mixture of CuBr₂ (15.13 g, 68 mmol, ground in a mortar-pestle) in EtOAc (45 mL) near reflux. Resulting mixture stirred vigorously at reflux under N₂ (balloon) for 3 hours, then cooled to room temperature. Reaction mixture suction filtered through paper and filtrate concentrated to give a solid that was triturated with 15% EtOAc-Hexanes (2×100 mL) and filtered. The washings were collected and concentrated and the resulting residue washed with 10% EtOAc-Hexanes (3×25 mL) leaving another crop of solid. The 2 solids obtained were combined, dissolved in CHCl₃ and suction filtered through paper. The filtrate was concentrated to give 2-bromo-1-(2-hydroxy-5-nitrophenyl)ethanone as an off-white solid, 4.74 g, 65% yield.

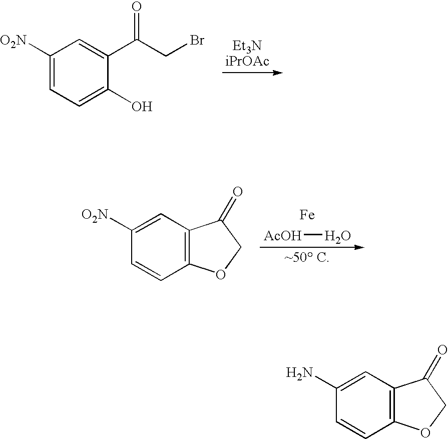

To a stirred solution of 2-bromo-1-(2-hydroxy-5-nitrophenyl)ethanone (4.74 g, 18 mmol) in isopropyl acetate (120 mL) was added triethylamine (2.53 mL, 19 mmol) at room temperature. Resulting mixture stirred for 90 minutes and then suction filtered through paper. The filtrate was concentrated and the crude product dissolved in EtOAc (60 mL) and used directly in the iron-mediated nitro reduction. A mixture of iron powder (5.02 g, 90 mmol, −325 mesh) in AcOH (25 mL) and H₂O (5 mL) stirred vigorously at 50° C. (oil bath) for 15 minutes. The flask was removed from the oil bath and additional H₂O (20 mL) added. To the warm, stirred mixture was added a solution of fresh 5-nitro-1-benzofuran-3(2H)-one in EtOAc in portions (˜2 mL portions) over a period of 20-25 minutes to maintain a slight exotherm. After addition was complete, the reaction mixture stirred for 5 minutes. H₂O (25 mL) was added, followed by EtOAc (150 mL). The mixture stirred vigorously for 10 seconds then EtOAc layer decanted off into aqueous Na₂CO₃ (46 g in 200 mL). Reaction mixture extracted further with EtOAc (6×50 mL) by stirring vigorously for 10 seconds then decanting into aqueous Na₂CO₃. Aqueous Na₂CO₃ layer extracted with EtOAc (100 mL). EtOAc extracts combined, washed with saturated aqueous NaCl (100 mL), dried over Na₂SO₄, decanted, and concentrated. Crude product immediately purified by silica gel chromatography using 20% EtOAc-CH₂Cl₂ to give the desired 5-amino-1-benzofuran-3(2H)-one, 2.25 g, 84% (2-steps) as a yellow solid.

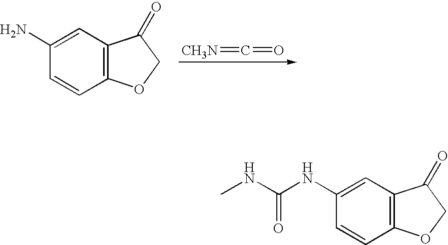

To a solution of 5-amino-1-benzofuran-3(2H)-one (450 mg, 3.0 mmol) in 50 mL of tetrahydrofuran was added methyl isocyanate (1M in toluene, 15 mL, 15 mmol). The mixture was stirred at room temperature for 3 days and filtered. The desired 1-methyl-3-(3-oxo-2,3-dihydro-1-benzofuran-5-yl)urea was obtained as a tan solid, 460 mg, 74% yield. MS: m/z 205.1 (MH−).

Preparation of methyl isocyanate: To a suspension of sodium azide (450 mg, 6.9 mmol) in 6.5 mL of toluene at 0° C. is added acetyl chloride (500 mg, 6.3 mmol). The mixture is heated at reflux with dry ice-acetone condenser cooling under nitrogen for 6 hrs, and cooled to room temperature. The supernatant is decanted, and used as 1.0 M methyl isocyanate solution in toluene.

### Suzuki-Coupling Procedure:

A mixture of the 3-formyl-2-bromoindole, boronic acid/ester (1-2 eq), Pd(OAc)₂ (3-5 mol %), PPh₃ (9-15 mol %) and K₃PO₄ (3 eq) in 1,2-dimethoxyethane and water was subjected to microwave conditions (155° C.). Reaction mixture cooled to room temperature, poured into water and extracted with EtOAc. EtOAc extracts combined, washed with saturated aqueous NaCl (25 mL), dried over Na₂SO₄, and concentrated. The mixture was purified by silica gel column chromatography.

### (Z)-1-(2-((5-Methoxy-2-(1,3,5-trimethyl-1H-pyrazol-4-yl)-1H-indol-3-yl)methylene)-3-oxo-2,3-dihydrobenzofuran-5-yl)-3-methylurea MS (m/z): 472.2 (MH⁺)

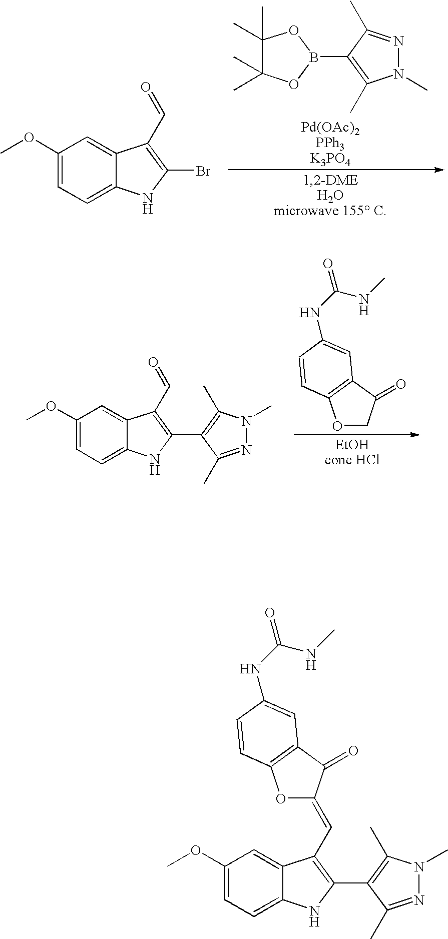

### Preparation of 5-methoxy-2-(1,3,5-trimethyl-1H-pyrazol-4-yl)-1H-indole-3-carbaldehyde

A mixture of 2-bromo-5-methoxy-1H-indole-3-carbaldehyde (132 mg, 0.52 mmol), 1,3,5-trimethyl-1-H-pyrazole-4-boronic acid pinacol ester (184 mg, 0.78 mmol), Pd(OAc)₂ (7 mg, 0.03 mmol), PPh₃ (24 mg, 0.09 mmol) and K₃PO₄ (331 mg, 1.56 mmol) in 1,2-dimethoxyethane (1.5 mL) and water (1.2 mL) was subjected to microwave conditions (155° C., 40 min). Reaction mixture cooled to room temperature, poured into water (25 mL) and extracted with EtOAc (2×50 mL). EtOAc extracts combined, washed with saturated aqueous NaCl (25 mL), dried over Na₂SO₄, and concentrated. The mixture purified by silica gel column chromatography (eluent: 80% EtOAc-hexanes). Yield >100%. MS (m/z): 284.2 (MH⁺).

### Preparation of (Z)-1-(2-((5-methoxy-2-(1,3,5-trimethyl-1H-pyrazol-4-yl)-1H-indol-3-yl)methylene)-3-oxo-2,3-dihydrobenzofuran-5-yl)-3-methylurea

Concentrated aqueous HCl (2 drops) was added to a stirred mixture of 5-methoxy-2-(1,3,5-trimethyl-1H-pyrazol-4-yl)-1H-indole-3-carbaldehyde (68 mg, 0.24 mmol) and 1-methyl-3-(3-oxo-2,3-dihydrobenzofuran-5-yl)urea (60 mg, 0.29 mmol) in EtOH (1.5 mL). Resulting mixture stirred at room temperature for 5 hours. EtOAc (2 mL) added and mixture filtered. Filtrate collected and concentrated. Crude product dissolved in EtOH (5 mL) and treated with saturated aqueous Na₂CO₃ (2 mL) and resulting mixture stirred at 75° C. for 15 minutes. The mixture cooled to room temperature, EtOAc (10 mL) added, organic layer collected and concentrated. Residue dissolved in EtOH (5 mL) and triturated with EtOAc (3 mL) then filtered. Filtrate concentrated and purified by preparative HPLC. Yield 35%. MS (m/z): 472.2 (MH⁺).

### (Z)-1-(2-((5-Methoxy-2-(1,3,5-trimethyl-1H-pyrazol-4-yl)-1H-indol-3-yl)methylene)-3-oxo-2,3-dihydrobenzofuran-5-yl)-3-(pyridin-3-yl)urea MS (m/z): 535.2 (MH⁺)

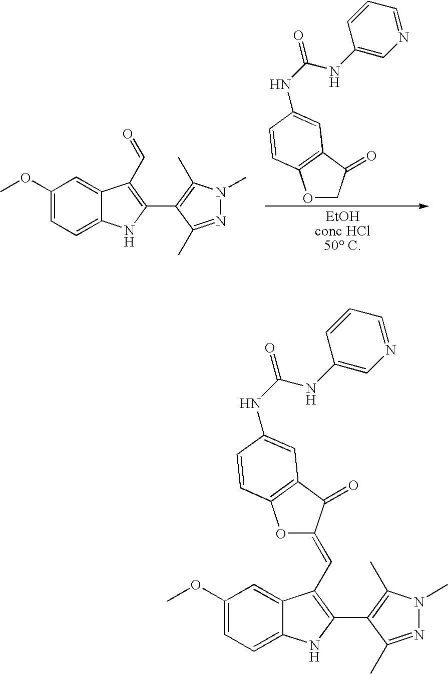

### (Z)-1-(2-((2-Bromo-5-methoxy-1H-indol-3-yl)methylene)-3-oxo-2,3-dihydrobenzofuran-5-yl)-3-methylurea MS (m/z): 442.0 (MH⁺)

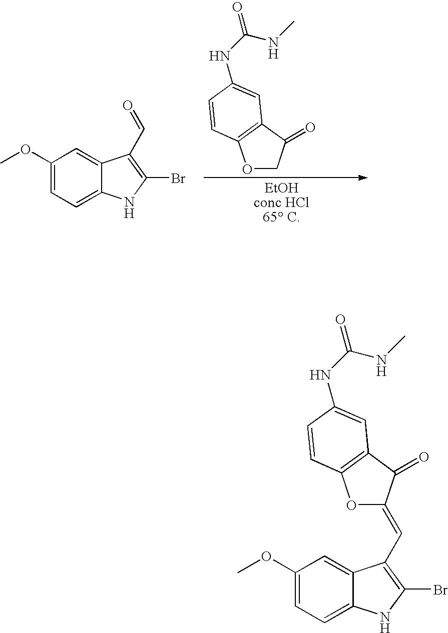

### Preparation of (Z)-1-(2-((7-fluoro-5-methoxy-2-(1,3,5-trimethyl-1H-pyrazol-4-yl)-1H-indol-3-yl)methylene)-3-oxo-2,3-dihydrobenzofuran-5-yl)-3-methyl urea

### 3-Formyl-2-bromoindoles via Gassman oxindole-Vilsmeier-Haack reactions

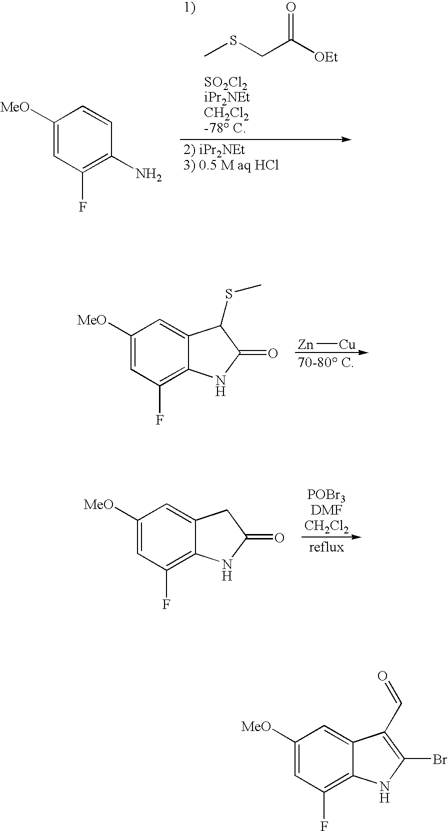

### Preparation of 7-fluoro-5-methoxy-3-(methylthio)indolin-2-one

Method similar to that referenced in J. Med. Chem. 2001, 44, 4339.

Sulfuryl chloride (3.05 mL, 38 mmol) added to a stirred solution of ethyl methylthioacetate (5.15 mL, 40 mmol) in CH₂Cl₂ (60 mL) at −78° C. over a period of 5 minutes. Resulting mixture stirred at −78° C. for 15 minutes then a solution of 2-fluoro-4-methoxyaniline (5.40 g, 38 mmol) and iPr₂NEt (6.62 mL, 38 mmol) in CH₂Cl₂ (60 mL) was added over a period of 45 minutes. Resulting mixture stirred at −78° C. for 30 minutes then iPr₂NEt (6.62 mL, 38 mmol) added over a period of 4 minutes. Cooling bath removed, mixture stirred overnight, and then solvent removed. Crude product dissolved in EtOAc (150 mL), 0.5 M aqueous HCl (150 mL) added, and the resulting mixture stirred overnight. Organic layer collected and aqueous layer extracted with EtOAc (2×150 mL). Organic layers combined, washed with water (50 mL), then saturated aqueous NaCl (2×50 mL), dried over Na₂SO₄ and concentrated. Resulting solid washed with 30% EtOAc-hexanes and then dried in vacuo. Yield 50%. MS (m/z): 226.1 (MH−).

### Preparation of 7-fluoro-5-methoxyindolin-2-one

A mixture of 7-fluoro-5-methoxy-3-(methylthio)indolin-2-one (4.35 g, 19 mmol) and zinc-copper couple (3.50 g) in AcOH (25 mL) and EtOAc (25 mL) was stirred at 70° C. for 2 hours, then overnight at 60° C. The mixture cooled to room temperature, diluted with EtOAc (100 mL) and suction filtered. Filtrate concentrated. Yield >100%. MS (m/z): 182.0 (MH⁺).

### Preparation of 2-bromo-7-fluoro-5-methoxy-1H-indole-3-carbaldehyde

A solution of POBr₃ (12.53 g, 44 mmol) in CH₂Cl₂ (50 mL) was added to a stirred solution of DMF (4.36 mL, 56 mmol) and CH₂Cl₂ (50 mL) over a period of 10 minutes. Resulting mixture stirred at reflux for 10 minutes and then a mixture of 7-fluoro-5-methoxyindolin-2-one (3.46 g, 19 mmol) in CH₂Cl₂ (30 mL) added over a period of 3 minutes. Resulting mixture stirred at reflux for 1 hour, cooled to room temperature and filtered. Filter cake rinsed with CH₂Cl₂ (2×50 mL) then the filter cake added to water (150 mL). The mixture swirled for 30 seconds, sat for 1 hour, and then extracted with EtOAc (4×100 mL). EtOAc extracts combined, washed with saturated aqueous NaCl (2×50 mL), dried over Na₂SO₄, and concentrated to give a solid. After sitting overnight, additional solid obtained from the aqueous layer by filtration and subsequent washing with water (3×25 mL). Solids combined and dried in vacuo. Yield 74%. MS (m/z): 271.9 (MH⁺).

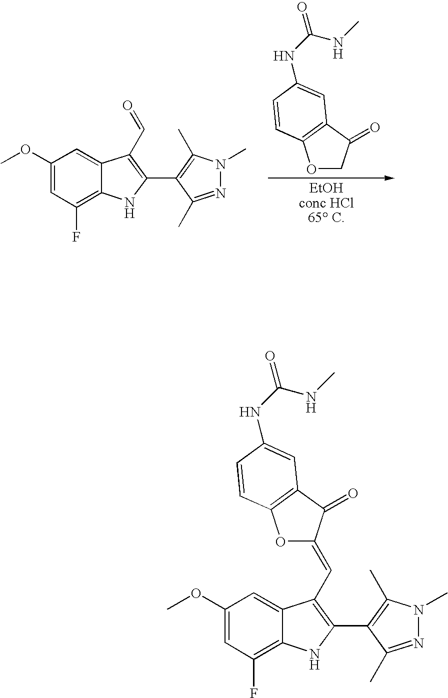

### Preparation of 7-fluoro-5-methoxy-2-(1,3,5-trimethyl-1H-pyrazol-4-yl)-1H-indole-3-carbaldehyde

A mixture of 2-bromo-7-fluoro-5-methoxy-1H-indole-3-carbaldehyde (269 mg, 0.99 mmol), 1,3,5-trimethyl-1-H-pyrazole-4-boronic acid pinacol ester (281 mg, 1.19 mmol), diacetoxy palladium (7 mg, 0.03 mmol), triphenylphosphine (24 mg, 0.09 mmol), and potassium phosphate (630 mg, 2.97 mmol) were treated with 1,2-dimethoxyethane (2.0 mL) and water (1.5 mL) then subjected to microwave conditions (155° C., 30 min). The mixture cooled to room temperature, diluted with water (25 mL) and extracted with EtOAc (3×50 mL). EtOAc extracts combined and washed with saturated aqueous NaCl (25 mL), dried over Na₂SO₄ and concentrated. Purified by silica gel chromatography (eluent: 80-100% EtOAc-hexanes gradient). Yield 75%. MS (m/z): 302.1 (MH⁺).

### Preparation of (Z)-1-(2-((7-fluoro-5-methoxy-2-(1,3,5-trimethyl-1H-pyrazol-4-yl)-1H-indol-3-yl)methylene)-3-oxo-2,3-dihydrobenzofuran-5-yl)-3-methyl urea

Concentrated aqueous HCl (4 drops) was added to a stirred mixture of 7-fluoro-5-methoxy-2-(1,3,5-trimethyl-1H-pyrazol-4-yl)-1H-indole-3-carbaldehyde (91 mg, 0.30 mmol) and 1-methyl-3-(3-oxo-2,3-dihydrobenzofuran-5-yl)urea (74 mg, 0.36 mmol) in EtOH (1.5 mL). Resulting mixture stirred at 65° C. for 3 hours, and then overnight at 60° C. The mixture cooled to room temperature and treated with saturated aqueous Na₂CO₃ (3 mL) and then stirred at 70° C. for 35 minutes. The mixture cooled to room temperature, diluted with EtOH (10 mL) and then filtered. Solid washed with water (3×5 mL) and EtOH (3 mL) and then dried in vacuo. Yield: 47%. MS (m/z): 490.2 (MH⁺).

### (Z)-1-(2-((2-(3,5-Dimethyl-1H-pyrazol-4-yl)-5-methoxy-1H-indol-3-yl)methylene)-3-oxo-2,3-dihydrobenzofuran-5-yl)-3-methylurea MS (m/z): 458.2 (MH⁺)

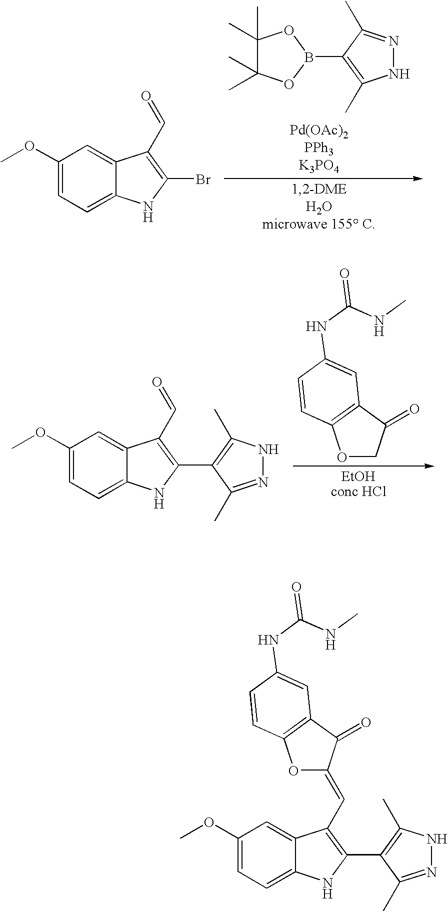

### Preparation of 2-(3,5-dimethyl-1H-pyrazol-4-yl)-5-methoxy-1H-indole-3-carbaldehyde

A mixture of 2-bromo-5-methoxy-1H-indole-3-carbaldehyde (129 mg, 0.51 mmol), 3,5-dimethylpyrazole-4-boronic acid pinacol ester (171 mg, 0.77 mmol), Pd(OAc)₂ (6 mg, 0.03 mmol), PPh₃ (31 mg, 0.12 mmol) and K₃PO₄ (325 mg, 1.53 mmol) in 1,2-dimethoxyethane (1.5 mL) and water (1 mL) was subjected to microwave conditions (155° C., 40 min). Additional Pd(OAc)₂ (6 mg, 0.03 mmol) and PPh₃ (30 mg, 0.12 mmol) added and reaction mixture re-subjected to microwave conditions (155° C., 50 min). Reaction mixture cooled to room temperature, poured into water (25 mL) and extracted with EtOAc (3×40 mL). EtOAc extracts combined, washed with saturated aqueous NaCl (25 mL), dried over Na₂SO₄, and concentrated. The mixture purified by silica gel column chromatography (eluent: 80-100% EtOAc-hexanes gradient). Yield 82%. MS (m/z): 270.2 (MH⁺).

### (Z)-1-(2-((2-(2,6-Dimethoxyphenyl)-5-methoxy-1H-indol-3-yl)methylene)-3-oxo-2,3-dihydrobenzofuran-5-yl)-3-methylurea MS (m/z): 500.2 (MH⁺)

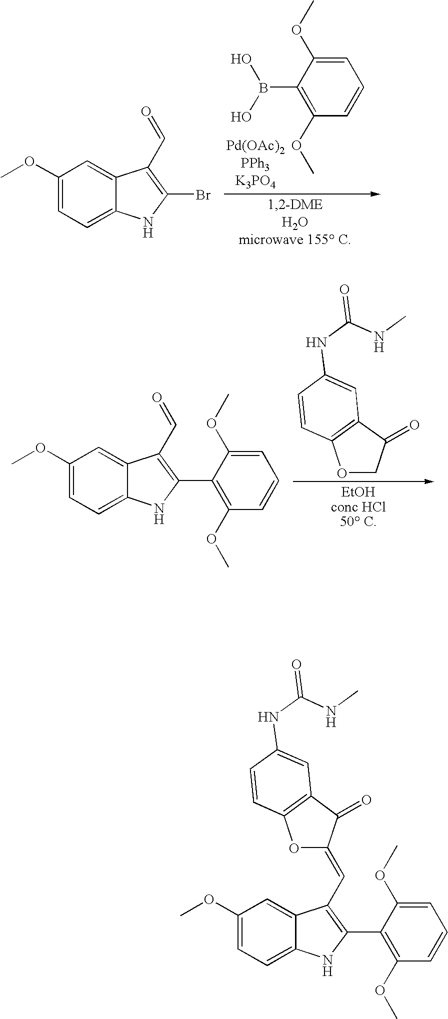

### Preparation of 2-(2,6-dimethoxyphenyl)-5-methoxy-1H-indole-3-carbaldehyde

A mixture of 2-bromo-5-methoxy-1H-indole-3-carbaldehyde (156 mg, 0.61 mmol), 2,6-dimethoxyphenyl boronic acid (133 mg, 0.73 mmol), Pd(OAc)₂ (4 mg, 0.02 mmol), PPh₃ (16 mg, 0.06 mmol), and K₃PO₄ (388 mg, 1.83 mmol) in 1,2-dimethoxyethane (1.5 mL) and water (1 mL) was subjected to microwave conditions (155° C., 30 min). Additional dimethoxyphenyl boronic acid (30 mg, 0.16 mmol) added and mixture re-subjected to microwave conditions (155° C., 15 min). Reaction mixture cooled to room temperature and diluted with EtOAc (5 mL). Organic layer collected, diluted with EtOAc (50 mL) and washed with saturated aqueous NaCl (25 mL), dried over Na₂SO₄ and concentrated. The mixture purified by silica gel column chromatography (eluent: 40-50% EtOAc-hexanes gradient). Yield 88%. MS (m/z): 312.1 (MH⁺).

### (Z)-1-(2-((2-(1-Isobutyl-1H-pyrazol-4-yl)-5-methoxy-1H-indol-3-yl)methylene)-3-oxo-2,3-dihydrobenzofuran-5-yl)-3-methylurea MS (m/z): 486.2 (MH⁺)

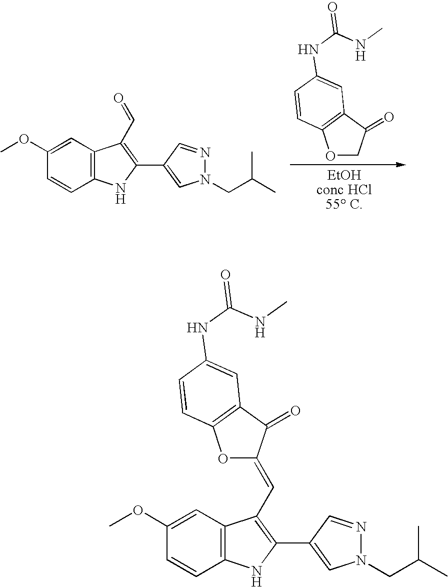

### Preparation of 2-(1-isobutyl-1H-pyrazol-4-yl)-5-methoxy-1H-indole-3-carbaldehyde

A mixture of 2-bromo-5-methoxy-1H-indole-3-carbaldehyde (206 mg, 0.81 mmol), 1-isobutyl-4-(4,4,5,5-tetramethyl-1,3,2-dioxaborolan-2-yl)-1H-pyrazole (243 mg, 0.97 mmol), diacetoxy palladium (7 mg, 0.03 mmol), triphenylphosphine (26 mg, 0.10 mmol), and potassium phosphate (516 mg, 2.43 mmol) were treated with DME (1.8 mL) and water (1.2 mL) then subjected to microwave conditions (155° C.) for 35 minutes. The mixture cooled to room temperature, poured into water (25 mL) and extracted with EtOAc (3×50 mL). EtOAc extracts combined and washed with saturated aqueous NaCl (25 mL), dried over Na₂SO₄ and concentrated. Purified by silica gel chromatography (eluent: 40-50% EtOAc-hexanes gradient). Yield 83%.

### (Z)-1-(2-((2-(1,3-Dimethyl-1H-pyrazol-4-yl)-5-methoxy-1H-indol-3-yl)methylene)-3-oxo-2,3-dihydrobenzofuran-5-yl)-3-methylurea MS (m/z): 458.2 (MH⁺)

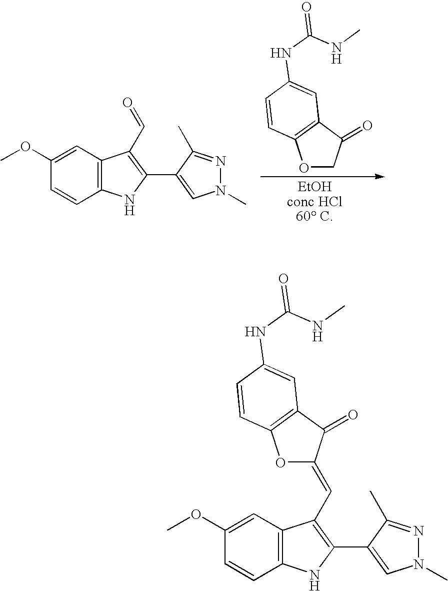

### Preparation of 2-(1,3-dimethyl-1H-pyrazol-4-yl)-5-methoxy-1H-indole-3-carbaldehyde

A mixture of 2-bromo-5-methoxy-1H-indole-3-carbaldehyde (315 mg, 1.24 mmol), 1,3-dimethyl-4-(4,4,5,5-tetramethyl-1,3,2-dioxaborolan-2-yl)-1H-pyrazole (303 mg, 1.36 mmol), diacetoxy palladium (9 mg, 0.04 mmol), triphenylphosphine (29 mg, 0.11 mmol), and potassium phosphate (789 mg, 3.72 mmol) in DME (2.5 mL) and water (1.5 mL) was subjected to microwave conditions (155° C., 30 min). Organic layer collected and concentrated. Residue purified by silica gel chromatography (eluent: 90% EtOAc-hexanes to 100% EtOAc gradient). Yield: 34%. MS (m/z): 270.1 (MH⁺).

### (Z)-1-(2-((2-(1,5-Dimethyl-1H-pyrazol-4-yl)-5-methoxy-1H-indol-3-yl)methylene)-3-oxo-2,3-dihydrobenzofuran-5-yl)-3-methylurea MS (m/z): 458.2 (MH⁺)

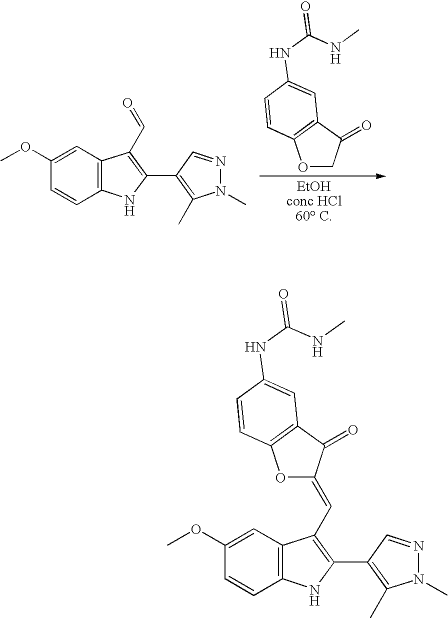

### Preparation of 2-(1,5-dimethyl-1H-pyrazol-4-yl)-5-methoxy-1H-indole-3-carbaldehyde

A mixture of 2-bromo-5-methoxy-1H-indole-3-carbaldehyde (315 mg, 1.24 mmol), 1,5-dimethyl-4-(4,4,5,5-tetramethyl-1,3,2-dioxaborolan-2-yl)-1H-pyrazole (303 mg, 1.36 mmol), diacetoxy palladium (9 mg, 0.04 mmol), triphenylphosphine (29 mg, 0.11 mmol), and potassium phosphate (789 mg, 3.72 mmol) in DME (2.5 mL) and water (1.5 mL) was subjected to microwave conditions (155° C., 30 min). Organic layer collected and concentrated. Residue purified by silica gel chromatography (eluent: 90% EtOAc-hexanes to 100% EtOAc gradient). Yield: 29%. MS (m/z): 270.1 (MH⁺).

### (Z)-1-(2-((5-Methoxy-2-(1-methyl-4-(trifluoromethyl)-1H-pyrazol-3-yl)-1H-indol-3-yl)methylene)-3-oxo-2,3-dihydrobenzofuran-5-yl)-3-methylurea MS (m/z): 512.2 (MH⁺)

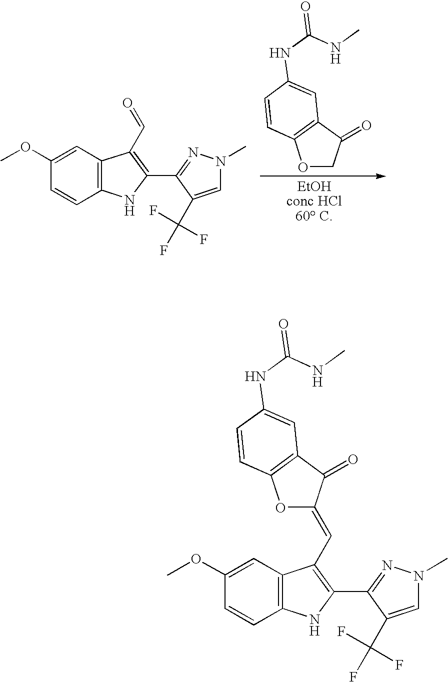

### Preparation of 5-methoxy-2-(1-methyl-4-(trifluoromethyl)-1H-pyrazol-3-yl)-1H-indole-3-carbaldehyde

A mixture of 2-bromo-5-methoxy-1H-indole-3-carbaldehyde (315 mg, 1.24 mmol), 1-methyl-3-(4,4,5,5-tetramethyl-1,3,2-dioxaborolan-2-yl)-4-(trifluoromethyl)-1H-pyrazole (376 mg, 1.36 mmol), diacetoxy palladium (9 mg, 0.040 mmol), triphenylphosphine (29 mg, 0.11 mmol), and potassium phosphate (789 mg, 3.72 mmol) in DME (2.5 mL) and water (1.5 mL) was subjected to microwave conditions (155° C., 30 min). EtOAc added (2 mL) to the cooled mixture and the organic layer collected and concentrated. Residue purified by silica gel chromatography (eluent: 30-35% EtOAc-hexanes gradient). Yield: 28%. MS (m/z): 324.1 (MH⁺).

### Preparation of (Z)-1-(2-((5-methoxy-2-(1-methyl-4-(trifluoromethyl)-1H-pyrazol-3-yl)-1H-indol-3-yl)methylene)-3-oxo-2,3-dihydrobenzofuran-5-yl)-3-methyl urea

Concentrated aqueous HCl (6 drops) was added to a stirred mixture of 5-methoxy-2-(1-methyl-4-(trifluoromethyl)-1H-pyrazol-3-yl)-1H-indole-3-carbaldehyde (108 mg, 0.33 mmol) and 1-methyl-3-(3-oxo-2,3-dihydrobenzofuran-5-yl)urea (69 mg, 0.33 mmol) in EtOH (2 mL). Resulting mixture stirred at 60° C. for 5 hours, then cooled to room temperature. The reaction mixture diluted with EtOH (10 mL) and then saturated aqueous Na₂CO₃ added (2 mL). Resulting mixture stirred for 5 minutes then filtered and the filtrate concentrated. Residue purified by silica gel chromatography (eluent: 70-100% EtOAc-hexanes gradient). Yield: 22%. MS (m/z): 512.2 (MH⁺).

### (Z)-1-(2-((5-Methoxy-2-(1-(2-methoxyethyl)-3,5-dimethyl-1H-pyrazol-4-yl)-1H-indol-3-yl)methylene)-3-oxo-2,3-dihydrobenzofuran-5-yl)-3-methylurea MS (m/z): 516.2 (MH⁺)

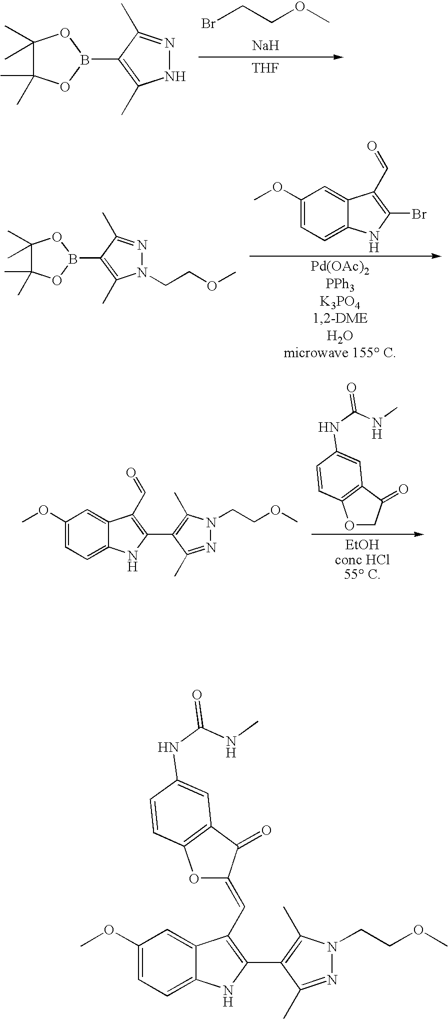

### Preparation of 1-(2-methoxyethyl)-3,5-dimethyl-4-(4,4,5,5-tetramethyl-1,3,2-dioxaborolan-2-yl)-1H-pyrazole

Sodium hydride (39 mg, 1.63 mmol) was added to a stirred solution of 3,5-dimethyl-4-(4,4,5,5-tetramethyl-1,3,2-dioxaborolan-2-yl)-1H-pyrazole (302 mg, 1.36 mmol) in THF (9.07 mL). Resulting mixture stirred for 5 minutes then 1-bromo-2-methoxyethane (153 μL, 1.63 mmol) added. Resulting mixture stirred for 30 minutes at room temperature then stirred overnight at 60° C. Additional NaH (˜50 mg) and 1-bromo-2-methoxyethane added (excess, 0.5 mL) and mixture heated to 68° C. for 3 hours. The reaction mixture cooled to room temperature, poured into H₂O (25 mL) and extracted with EtOAc (3×25 mL). EtOAc extracts combined, washed with saturated aqueous NaCl (25 mL), dried over Na₂SO₄, and concentrated. Crude product dissolved in hexanes (10 mL) and mixture sat for 15 minutes, filtered, and the filtrate collected and concentrated. Product used immediately.

### Preparation of 5-methoxy-2-(1-(2-methoxyethyl)-3,5-dimethyl-1H-pyrazol-4-yl)-1H-indole-3-carbaldehyde

A mixture of 2-bromo-5-methoxy-1H-indole-3-carbaldehyde (185 mg, 0.72 mmol), 1-(2-methoxyethyl)-3,5-dimethyl-4-(4,4,5,5-tetramethyl-1,3,2-dioxaborolan-2-yl)-1H-pyrazole (225 mg, 0.80 mmol), diacetoxy palladium (7 mg, 0.03 mmol), triphenylphosphine (23 mg, 0.09 mmol), and potassium phosphate (464 mg, 2.18 mmol) in DME (1.5 mL) and water (1 mL) was subjected to microwave conditions (155° C.) for 35 minutes. The mixture cooled to room temperature, poured into water (25 mL) and extracted with EtOAc (3×50 mL). EtOAc extracts combined, washed with saturated aqueous NaCl (25 mL), dried over Na₂SO₄, and concentrated. Product purified by silica gel chromatography (eluent: 85-100% EtOAc-hexanes gradient). Yield: 60%. MS (m/z): 328.2 (MH⁺).

### Preparation of (Z)-1-(2-((5-methoxy-2-(1-(2-methoxyethyl)-3,5-dimethyl-1H-pyrazol-4-yl)-1H-indol-3-yl)methylene)-3-oxo-2,3-dihydrobenzofuran-5-yl)-3-methylurea

Concentrated aqueous HCl (5 drops) was added to a stirred mixture of 5-methoxy-2-(1-(2-methoxyethyl)-3,5-dimethyl-1H-pyrazol-4-yl)-1H-indole-3-carbaldehyde (70 mg, 0.21 mmol) and 1-methyl-3-(3-oxo-2,3-dihydrobenzofuran-5-yl)urea (53 mg, 0.26 mmol) in EtOH (1.5 mL). Resulting mixture stirred overnight at 55° C. The mixture cooled to room temperature, diluted with additional EtOH (5 mL) then added to saturated aqueous Na₂CO₃ (5 mL) and then stirred at 65° C. for 20 minutes. Deep red organic layer was collected and concentrated. Residue dissolved in MeOH, filtered, and subjected to preparative HPLC. Yield: 35%. MS (m/z): 516.2 (MH⁺).

### (Z)-1-(2-((2-(1-(2-(Dimethylamino)ethyl)-3,5-dimethyl-1H-pyrazol-4-yl)-5-methoxy-1H-indol-3-yl)methylene)-3-oxo-2,3-dihydrobenzofuran-5-yl)-3-methyl urea MS (m/z): 529.3 (MH⁺)

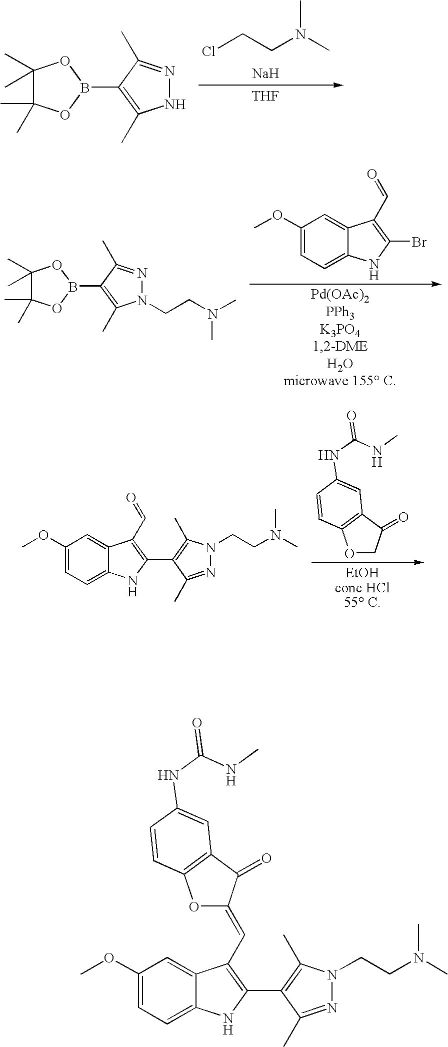

### Preparation of 2-(3,5-dimethyl-4-(4,4,5,5-tetramethyl-1,3,2-dioxaborolan-2-yl)-1H-pyrazol-1-yl)-N,N-dimethylethanamine

Sodium hydride (40 mg, 1.69 mmol) was added to a stirred solution of 3,5-dimethyl-4-(4,4,5,5-tetramethyl-1,3,2-dioxaborolan-2-yl)-1H-pyrazole (313 mg, 1.41 mmol) in THF (9.40 mL). Resulting mixture stirred for 5 minutes then 2-chloro-N,N-dimethylethanamine (182 mg, 1.69 mmol) added. (2-Chloro-N,N-dimethylethanamine was prepared from its corresponding HCl salt by partitioning between 20% Et₂O-Hex and 5 M aqueous NaOH, drying the organic layer over Na₂SO₄, removal of solvent, and then using the resulting residue directly). Resulting mixture stirred for 30 minutes at room temperature, then stirred overnight at 60° C. The mixture poured into 1:1 H₂O-saturated aqueous NaCl (25 mL) and extracted with EtOAc (2×50 mL). EtOAc layers combined, washed with saturated aqueous NaCl (25 mL), dried over Na₂SO₄, and concentrated to give an oil. The oil was triturated with hexanes (15 mL) and filtered. Filtrate collected and concentrated in vacuo. Product used immediately.

### Preparation of 2-(1-(2-(dimethylamino)ethyl)-3,5-dimethyl-1H-pyrazol-4-yl)-5-methoxy-1H-indole-3-carbaldehyde

A mixture of 2-bromo-5-methoxy-1H-indole-3-carbaldehyde (240 mg, 0.94 mmol), 2-(3,5-dimethyl-4-(4,4,5,5-tetramethyl-1,3,2-dioxaborolan-2-yl)-1H-pyrazol-1-yl)-N,N-dimethylethanamine (336 mg, 1.15 mmol), diacetoxy palladium (8 mg, 0.04 mmol), triphenylphosphine (30 mg, 0.11 mmol), and potassium phosphate (602 mg, 2.83 mmol) in DME (2.2 mL) and water (1.4 mL) was subjected to microwave conditions (155° C., 30 min). The mixture cooled to room temperature, poured into 1 M aqueous HCl (25 mL) and EtOAc (50 mL). Organic layer extracted with 1 M aqueous HCl (25 mL). Aqueous layers combined and extracted with EtOAc (2×25 mL), then basified to pH˜8-9 using saturated aqueous Na₂CO₃. Basified aqueous layer extracted with EtOAc (3×50 mL). EtOAc extracts of the basic aqueous layer were combined, washed with saturated aqueous NaCl (25 mL), dried over Na₂SO₄, and concentrated. Yield: 77%. MS (m/z): 341.4 (MH⁺).

### Preparation of (Z)-1-(2-((2-(1-(2-(dimethylamino)ethyl)-3,5-dimethyl-1H-pyrazol-4-yl)-5-methoxy-1H-indol-3-yl)methylene)-3-oxo-2,3-dihydrobenzofuran-5-yl)-3-methylurea

Concentrated aqueous HCl (6 drops) was added to a stirred mixture of 2-(1-(2-(dimethylamino)ethyl)-3,5-dimethyl-1H-pyrazol-4-yl)-5-methoxy-1H-indole-3-carbaldehyde (100 mg, 0.29 mmol) and 1-methyl-3-(3-oxo-2,3-dihydrobenzofuran-5-yl)urea (73 mg, 0.35 mmol) in EtOH (2 mL). Resulting mixture stirred at 55° C. for 3 hours, then at room temperature overnight. EtOAc (3 mL) added and mixture suction filtered through sintered glass. Filtrate collected and concentrated. Crude product purified by preparative HPLC. Yield: 52%. MS (m/z): 529.3 (MH⁺)

### (Z)-1-(2-((1-(3-Cyanopropyl)-5-methoxy-2-(1,3,5-trimethyl-1H-pyrazol-4-yl)-1H-indol-3-yl)methylene)-3-oxo-2,3-dihydrobenzofuran-5-yl)-3-methyl urea MS (m/z): 539.2 (M H⁺)

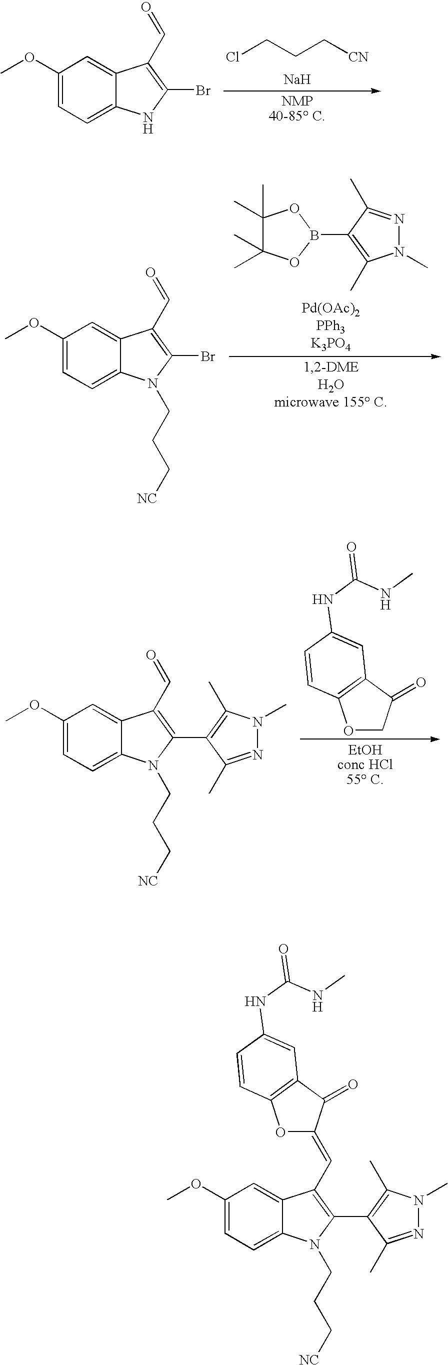

### Preparation of 4-(2-bromo-3-formyl-5-methoxy-1H-indol-1-yl)butanenitrile

A solution of 2-bromo-5-methoxy-1H-indole-3-carbaldehyde (100 mg, 0.39 mmol) in NMP (1.2 mL) added slowly to NaH (excess) at room temperature. Resulting mixture stirred for 25 minutes then 4-chlorobutyronitrile (46 μL, 0.51 mmol) added. Reaction mixture heated to 40° C. and stirred for 90 minutes, then stirred overnight at 85° C. The mixture cooled to room temperature, poured into saturated aqueous NaCl (25 mL) and extracted with EtOAc (2×50 mL). EtOAc extracts combined, washed with saturated aqueous NaCl (2×25 mL), dried over Na₂SO₄ and concentrated. The mixture purified by silica gel column chromatography (eluent: 20-35% EtOAc-hexanes gradient). Yield 88%. MS (m/z): 321.0 (MH⁺).

### Preparation of 4-(3-formyl-5-methoxy-2-(1,3,5-trimethyl-1H-pyrazol-4-yl)-1H-indol-1-yl)butanenitrile

A mixture of 4-(2-bromo-3-formyl-5-methoxy-1H-indol-1-yl)butanenitrile (102 mg, 0.32 mmol), 1,3,5-trimethyl-1-H-pyrazole-4-boronic acid pinacol ester (106 mg, 0.45 mmol), Pd(OAc)₂ (3 mg, 0.01 mmol), PPh₃ (10 mg, 0.04 mmol), K₃PO₄ (204 mg, 0.96 mmol) in 1,2-dimethoxyethane (1.5 mL) and water (1 mL) was subjected to microwave conditions (155° C., 40 min). Reaction mixture cooled to room temperature, poured into water (25 mL) and extracted with EtOAc (2×50 mL). EtOAc extracts combined, washed with saturated aqueous NaCl (25 mL), dried over Na₂SO₄, and concentrated. The mixture purified by silica gel column chromatography (eluent: 75-100% EtOAc-hexanes). Yield 68%. MS (m/z): 351.2 (MH⁺).

### Preparation of (Z)-1-(2-((1-(3-cyanopropyl)-5-methoxy-2-(1,3,5-trimethyl-1H-pyrazol-4-yl)-1H-indol-3-yl)methylene)-3-oxo-2,3-dihydrobenzofuran-5-yl)-3-methylurea

Concentrated aqueous HCl (3 drops) was added to a stirred mixture of 4-(3-formyl-5-methoxy-2-(1,3,5-trimethyl-1H-pyrazol-4-yl)-1H-indol-1-yl)butanenitrile (70 mg, 0.20 mmol) and 1-methyl-3-(3-oxo-2,3-dihydrobenzofuran-5-yl)urea (49 mg, 0.24 mmol) in EtOH (1.5 mL). Resulting mixture stirred overnight at 40° C. and then 55° C. for 5 hours. Reaction mixture stored at 5° C. for 1 week. EtOAc (2 mL) added and mixture filtered. Filtrate treated with K₂CO₃ (300 mg) and diluted with EtOH (5 mL) and water (0.5 mL). Resulting mixture stirred at 70° C. for 15 minutes then cooled to room temperature. Organic layer collected and concentrated. Residue purified by preparative HPLC. Yield 24%. MS (m/z): 539.2 (MH⁺).

### (Z)-1-(2-((5-Methoxy-2-(4-methylpiperazin-1-yl)-1-(2-(4-methylpiperazin-1-yl)ethyl)-1H-indol-3-yl)methylene)-3-oxo-2,3-dihydrobenzofuran-5-yl)-3-methyl urea MS (m/z): 588.3 (M H⁺)

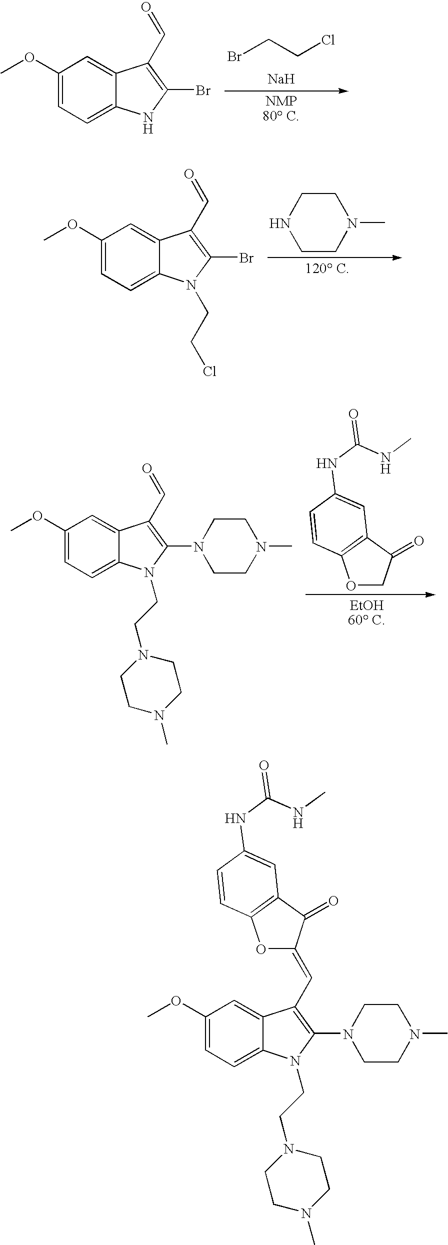

### Preparation of 2-bromo-1-(2-chloroethyl)-5-methoxy-1H-indole-3-carbaldehyde

A solution of 2-bromo-5-methoxy-1H-indole-3-carbaldehyde (300 mg, 1.18 mmol) in NMP (2 mL) was added to NaH (40 mg, 1.67 mmol) over a period of 1 minutes. Resulting mixture stirred at room temperature for 30 minutes, then at 80° C. for 10 minutes. 1-Bromo-2-chloroethane (490 μL, 5.9 mmol) was added and reaction mixture stirred at 80° C. for 5 hours. Reaction mixture cooled to room temperature, poured into saturated aqueous NaCl (25 mL) and extracted with EtOAc (100 mL). EtOAc layer washed with saturated aqueous NaCl (3×25 mL), dried over Na₂SO₄ and concentrated. Yield 100%. MS (m/z): 316.0 (MH⁺).

### Preparation of 5-methoxy-2-(4-methylpiperazin-1-yl)-1-(2-(4-methylpiperazin-1-yl)ethyl)-1H-indole-3-carbaldehyde

A solution of 2-bromo-1-(2-chloroethyl)-5-methoxy-1H-indole-3-carbaldehyde (365 mg, 1.15 mmol) in 1-methylpiperazine (3 mL) was heated to 105° C. for 2.5 hours, then 120° C. for 2 hours. Reaction mixture cooled to room temperature, poured into 1:1 saturated aqueous NaCl—H₂O (40 mL) and extracted with EtOAc (2×50 mL). EtOAc layers combined, dried over Na₂SO₄ and concentrated.

### Preparation of (Z)-1-(2-((5-methoxy-2-(4-methylpiperazin-1-yl)-1-(2-(4-methylpiperazin-1-yl)ethyl)-1H-indol-3-yl)methylene)-3-oxo-2,3-dihydrobenzofuran-5-yl)-3-methylurea

A mixture of 5-methoxy-2-(4-methylpiperazin-1-yl)-1-(2-(4-methylpiperazin-1-yl)ethyl)-1H-indole-3-carbaldehyde (95 mg, 0.24 mmol) and 1-methyl-3-(3-oxo-2,3-dihydrobenzofuran-5-yl)urea (52 mg, 0.25 mmol) in EtOH (1.5 mL) was stirred at 60° C. for 2 days. Reaction mixture cooled to room temperature and purified directly by silica gel column chromatography (eluent: 70:20:10 CH₃CN-Et₃N-MeOH). Yield 47%. MS (m/z): 588.3 (MH⁺).

### (Z)-1-(2-((5-Methoxy-1-(2-(4-methylpiperazin-1-yl)ethyl)-2-(1,3,5-trimethyl-1H-pyrazol-4-yl)-1H-indol-3-yl)methylene)-3-oxo-2,3-dihydrobenzofuran-5-yl)-3-methyl urea MS (m/z): 598.3 (M H⁺)

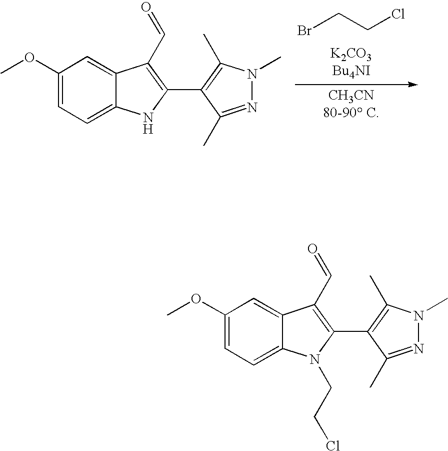

### Preparation of 1-(2-chloroethyl)-5-methoxy-2-(1,3,5-trimethyl-1H-pyrazol-4-yl)-1H-indole-3-carbaldehyde

A mixture of 5-methoxy-2-(1,3,5-trimethyl-1H-pyrazol-4-yl)-1H-indole-3-carbaldehyde (327 mg, 1.15 mmol), 1-bromo-2-chloroethane (765 μL, 9.23 mmol), K₂CO₃ (1.12 g, 8.1 mmol), and Bu₄NI (40 mg) in CH₃CN (5.8 mL) was stirred at 80° C. overnight. The mixture cooled to room temperature, poured into water (25 mL) and extracted with EtOAc (3×50 mL). EtOAc extracts combined, washed with saturated aqueous NaCl (25 mL), dried over Na₂SO₄, and concentrated. The mixture purified by silica gel column chromatography (eluent: 80-100% EtOAc-hexanes gradient). Yield 93%.

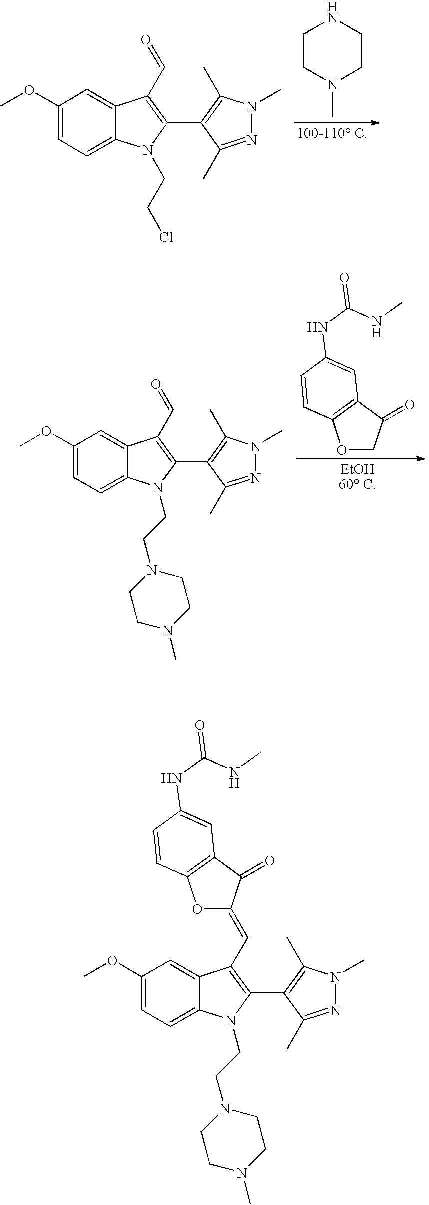

### Preparation of 5-methoxy-1-(2-(4-methylpiperazin-1-yl)ethyl)-2-(1,3,5-trimethyl-1H-pyrazol-4-yl)-1H-indole-3-carbaldehyde

A mixture of 1-(2-chloroethyl)-5-methoxy-2-(1,3,5-trimethyl-1H-pyrazol-4-yl)-1H-indole-3-carbaldehyde (90 mg) and 1-methylpiperazine (2.5 mL) was heated between 100-110° C. over a total period of 12 hours. Reaction mixture cooled to room temperature and concentrated in vacuo. Crude product partitioned between EtOAc and 0.5 M aqueous HCl. Aqueous layer extracted twice with EtOAc. Aqueous layer made basic (pH 9) using saturated aqueous Na₂CO₃, then extracted with EtOAc (3×). EtOAc extracts of basic aqueous layer combined, washed with saturated aqueous NaCl, dried over Na₂SO₄, and concentrated. Yield 40%. MS (m/z): 410.2 (MH⁺).

### Preparation of (Z)-1-(2-((5-methoxy-1-(2-(4-methylpiperazin-1-yl)ethyl)-2-(1,3,5-trimethyl-1H-pyrazol-4-yl)-1H-indol-3-yl)methylene)-3-oxo-2,3-dihydrobenzofuran-5-yl)-3-methylurea

Concentrated aqueous HCl (6 drops) was added to a stirred mixture of 5-methoxy-1-(2-(4-methylpiperazin-1-yl)ethyl)-2-(1,3,5-trimethyl-1H-pyrazol-4-yl)-1H-indole-3-carbaldehyde (48 mg, 0.12 mmol) and 1-methyl-3-(3-oxo-2,3-dihydrobenzofuran-5-yl)urea (29 mg, 0.14 mmol) in EtOH (1.0 mL). Resulting mixture stirred at 60° C. for 3 hours. The mixture cooled to room temperature, diluted with additional EtOH (5 mL) then added to saturated aqueous Na₂CO₃ (3 mL) then stirred at 65° C. for 25 minutes. The mixture cooled to room temperature, diluted with EtOH (10 mL), filtered, and filtrate collected and concentrated. Residue purified by preparative HPLC. Yield: 31%. MS (m/z): 598.3 (MH⁺).

### (Z)-1-(2-((1-(2-(2-Hydroxyethylamino)ethyl)-5-methoxy-2-(1,3,5-trimethyl-1H-pyrazol-4-yl)-1H-indol-3-yl)methylene)-3-oxo-2,3-dihydrobenzofuran-5-yl)-3-methyl urea MS (m/z): 559.3 (M H⁺)

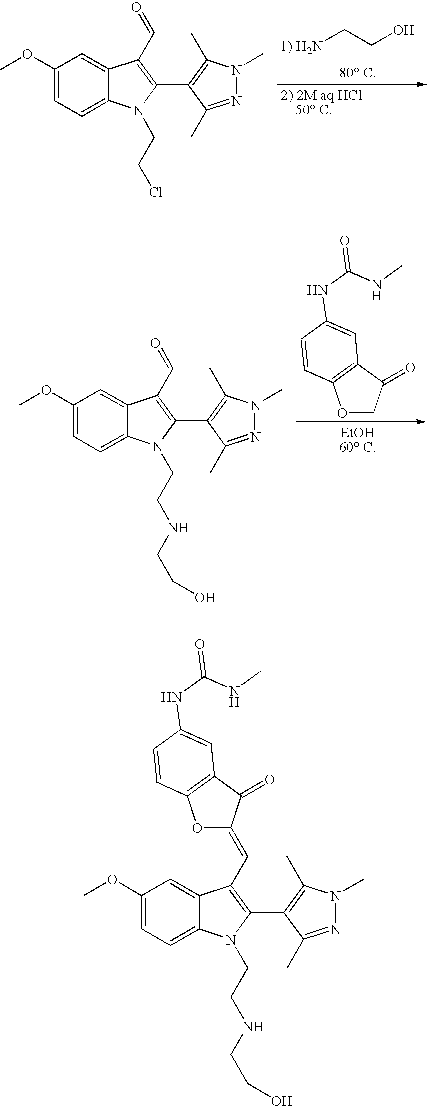

### Preparation of 1-(2-(2-hydroxyethylamino)ethyl)-5-methoxy-2-(1,3,5-trimethyl-1H-pyrazol-4-yl)-1H-indole-3-carbaldehyde

A mixture of 1-(2-chloroethyl)-5-methoxy-2-(1,3,5-trimethyl-1H-pyrazol-4-yl)-1H-indole-3-carbaldehyde (112 mg) and ethanolamine (2 mL) was heated to 80° C. overnight. Reaction mixture cooled to room temperature and 2 M aqueous HCl (30 mL) added and resulting mixture stirred at 50° C. for 90 minutes. The mixture cooled to room temperature and made basic (pH 8-9) using saturated aqueous Na₂CO₃ and extracted with EtOAc (3×40 mL). EtOAc extracts combined, washed with 1:1 saturated aqueous NaCl-water (2×15 mL), then saturated aqueous NaCl (25 mL), dried over Na₂SO₄ and concentrated. MS (m/z): 371.2 (MH⁺).

### Preparation of (Z)-1-(2-((1-(2-(2-hydroxyethylamino)ethyl)-5-methoxy-2-(1,3,5-trimethyl-1H-pyrazol-4-yl)-1H-indol-3-yl)methylene)-3-oxo-2,3-dihydrobenzofuran-5-yl)-3-methylurea

Concentrated aqueous HCl (5 drops) was added to a stirred mixture of 1-(2-(2-hydroxyethylamino)ethyl)-5-methoxy-2-(1,3,5-trimethyl-1H-pyrazol-4-yl)-1H-indole-3-carbaldehyde (80 mg, 0.22 mmol) and 1-methyl-3-(3-oxo-2,3-dihydrobenzofuran-5-yl)urea (54 mg, 0.26 mmol) in EtOH (1.0 mL). Resulting mixture stirred at 60° C. for 2 hours. The mixture cooled to room temperature, diluted with additional EtOH (5 mL), and then neutralized using saturated aqueous Na₂CO₃. The mixture filtered, and filtrate collected and concentrated. Residue purified by preparative HPLC. Yield: 9%. MS (m/z): 559.3 (MH⁺).

### (Z)-1-(2-((1-(2-((2-(Dimethylamino)ethyl)(methyl)amino)ethyl)-5-methoxy-2-(1,3,5-trimethyl-1H-pyrazol-4-yl)-1H-indol-3-yl)methylene)-3-oxo-2,3-dihydrobenzofuran-5-yl)-3-methyl urea MS (m/z): 600.3 (MH⁺)

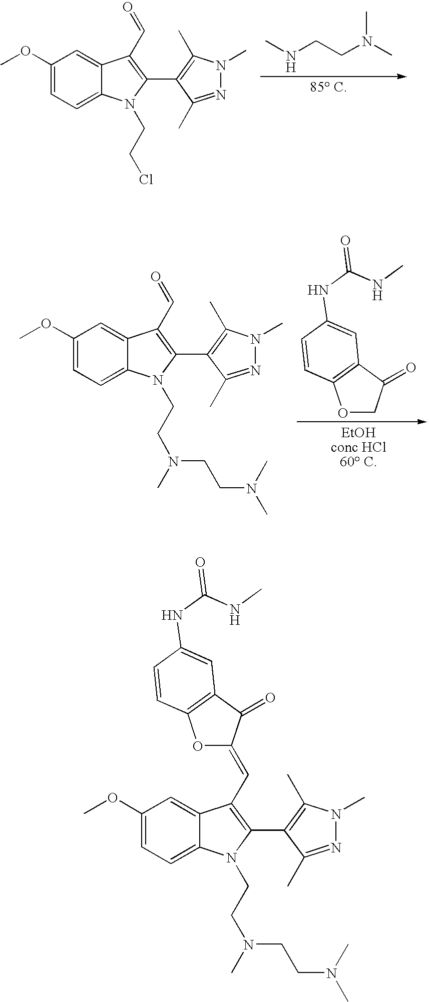

### Preparation of 1-(2-((2-(dimethylamino)ethyl)(methyl)amino)ethyl)-5-methoxy-2-(1,3,5-trimethyl-1H-pyrazol-4-yl)-1H-indole-3-carbaldehyde

A mixture of 1-(2-chloroethyl)-5-methoxy-2-(1,3,5-trimethyl-1H-pyrazol-4-yl)-1H-indole-3-carbaldehyde (112 mg, 0.32 mmol) and N,N,N′-trimethylethylenediamine (2 mL) was heated to 85° C. overnight. The mixture cooled to room temperature and treated with 1 M aqueous HCl (25 mL), diluted with water (10 mL), and extracted with EtOAc (2×40 mL). Aqueous layer made basic (pH 8-9) using saturated aqueous Na₂CO₃ and extracted with EtOAc (3×40 mL). EtOAc extracts of the basic aqueous layer combined, washed with saturated aqueous NaCl (25 mL), dried over Na₂SO₄, and concentrated. Yield: 61%. MS (m/z): 412.3 (MH⁺).

### Preparation of (Z)-1-(2-((1-(2-((2-(dimethylamino)ethyl)(methyl)amino)ethyl)-5-methoxy-2-(1,3,5-trimethyl-1H-pyrazol-4-yl)-1H-indol-3-yl)methylene)-3-oxo-2,3-dihydrobenzofuran-5-yl)-3-methylurea

Concentrated aqueous HCl (5 drops) was added to a stirred mixture of 1-(2-((2-(dimethylamino)ethyl)(methyl)amino)ethyl)-5-methoxy-2-(1,3,5-trimethyl-1H-pyrazol-4-yl)-1H-indole-3-carbaldehyde (78 mg, 0.19 mmol) and 1-methyl-3-(3-oxo-2,3-dihydrobenzofuran-5-yl)urea (47 mg, 0.23 mmol) in EtOH (1.2 mL). Resulting mixture stirred overnight at 50° C. Additional 1-methyl-3-(3-oxo-2,3-dihydrobenzofuran-5-yl)urea (25 mg, 0.12 mmol) added and mixture stirred at 60° C. for 4 hours. The mixture cooled to room temperature and made basic (pH˜9) using saturated aqueous Na₂CO₃. Resulting mixture stirred at 65° C. for 30 minutes, cooled to room temperature, diluted with EtOH (10 mL), poured into EtOAc (50 mL), and then filtered. Filtrate collected and concentrated. Residue purified by preparative HPLC. Yield: 13%. MS (m/z): 600.3 (MH⁺).

### (Z)-1-(2-((1-(2-(2-(Dimethylamino)ethylamino)ethyl)-5-methoxy-2-(1,3,5-trimethyl-1H-pyrazol-4-yl)-1H-indol-3-yl)methylene)-3-oxo-2,3-dihydrobenzofuran-5-yl)-3-methyl urea MS (m/z): 586.3 (MH⁺)

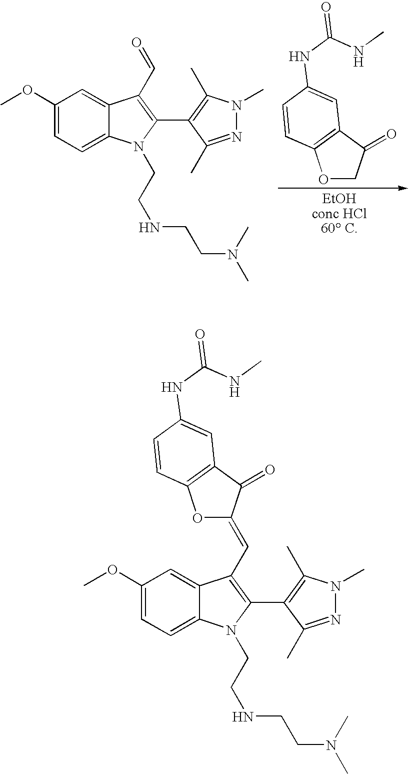

### Preparation of 1-(2-(2-(dimethylamino)ethylamino)ethyl)-5-methoxy-2-(1,3,5-trimethyl-1H-pyrazol-4-yl)-1H-indole-3-carbaldehyde

Method as described for the preparation of 1-(2-((2-(dimethylamino)ethyl)(methyl)amino)ethyl)-5-methoxy-2-(1,3,5-trimethyl-1H-pyrazol-4-yl)-1H-indole-3-carbaldehyde except using N,N-dimethylethylenediamine as the amine. Yield: 89%. MS (m/z): 398.3 (MH⁺).

### Preparation of (Z)-1-(2-((1-(2-(2-(dimethylamino)ethylamino)ethyl)-5-methoxy-2-(1,3,5-trimethyl-1H-pyrazol-4-yl)-1H-indol-3-yl)methylene)-3-oxo-2,3-dihydrobenzofuran-5-yl)-3-methylurea

Concentrated aqueous HCl (7 drops) was added to a stirred mixture of 1-(2-(2-(dimethylamino)ethylamino)ethyl)-5-methoxy-2-(1,3,5-trimethyl-1H-pyrazol-4-yl)-1H-indole-3-carbaldehyde (112 mg, 0.28 mmol) and 1-methyl-3-(3-oxo-2,3-dihydrobenzofuran-5-yl)urea (70 mg, 0.34 mmol) in EtOH (2 mL). Resulting mixture stirred overnight at 50° C. and then at 60° C. for 4 hours. The mixture cooled to room temperature and made basic (pH˜9) using saturated aqueous Na₂CO₃. Resulting mixture stirred at 65° C. for 30 minutes, cooled to room temperature, diluted with EtOH (10 mL), poured into EtOAc (50 mL), and then filtered. Filtrate collected and concentrated. Residue purified by preparative HPLC. Yield: 20%. MS (m/z): 586.3 (MH⁺).

### (Z)-1-(2-((5-Methoxy-1-(2-(piperazin-1-yl)ethyl)-2-(1,3,5-trimethyl-1H-pyrazol-4-yl)-1H-indol-3-yl)methylene)-3-oxo-2,3-dihydrobenzofuran-5-yl)-3-methyl urea MS (m/z): 584.3 (M H⁺)

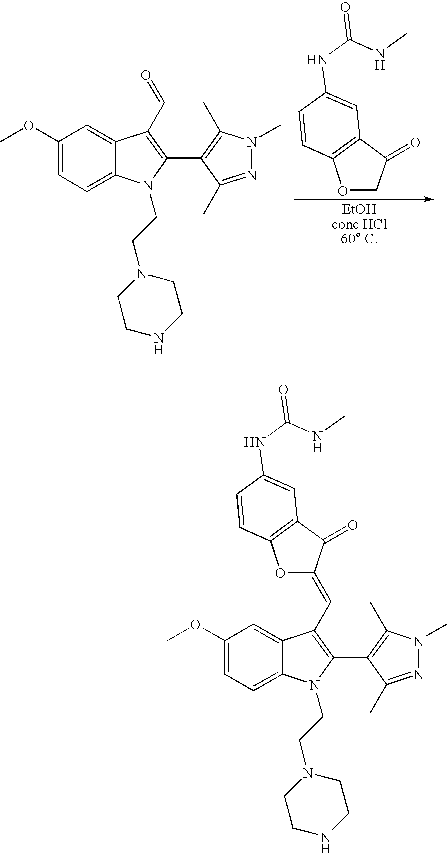

### Preparation of 5-methoxy-1-(2-(piperazin-1-yl)ethyl)-2-(1,3,5-trimethyl-1H-pyrazol-4-yl)-1H-indole-3-carbaldehyde

Method as described for the preparation of 1-(2-((2-(dimethylamino)ethyl)(methyl)amino)ethyl)-5-methoxy-2-(1,3,5-trimethyl-1H-pyrazol-4-yl)-1H-indole-3-carbaldehyde except using piperazine as the amine. Yield: 83%. MS (m/z): 396.3 (MH⁺).

### Preparation of (Z)-1-(2-((5-methoxy-1-(2-(piperazin-1-yl)ethyl)-2-(1,3,5-trimethyl-1H-pyrazol-4-yl)-1H-indol-3-yl)methylene)-3-oxo-2,3-dihydrobenzofuran-5-yl)-3-methylurea

Concentrated aqueous HCl (7 drops) was added to a stirred mixture of 5-methoxy-1-(2-(piperazin-1-yl)ethyl)-2-(1,3,5-trimethyl-1H-pyrazol-4-yl)-1H-indole-3-carbaldehyde (127 mg, 0.32 mmol) and 1-methyl-3-(3-oxo-2,3-dihydrobenzofuran-5-yl)urea (78 mg, 0.38 mmol) in EtOH (2.4 mL). Resulting mixture stirred overnight at 50° C. and then at 60° C. for 4 hours. The mixture cooled to room temperature and made basic (pH˜9) using saturated aqueous Na₂CO₃. Resulting mixture stirred at 65° C. for 30 minutes, cooled to room temperature, diluted with EtOH (10 mL), poured into EtOAc (50 mL), and then filtered. Filtrate collected and concentrated. Residue purified by preparative HPLC. Yield: 14%. MS (m/z): 584.3 (MH⁺).

### (Z)-1-(2-((5-Methoxy-1-(2-(methylamino)ethyl)-2-(1,3,5-trimethyl-1H-pyrazol-4-yl)-1H-indol-3-yl)methylene)-3-oxo-2,3-dihydrobenzofuran-5-yl)-3-methyl urea MS (m/z): 529.3 (M H⁺)

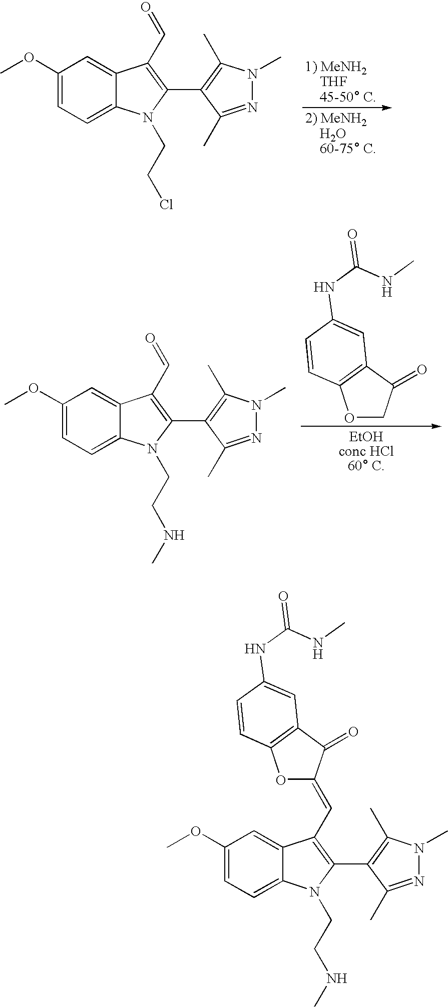

### Preparation of 5-methoxy-1-(2-(methylamino)ethyl)-2-(1,3,5-trimethyl-1H-pyrazol-4-yl)-1H-indole-3-carbaldehyde

A solution of 1-(2-chloroethyl)-5-methoxy-2-(1,3,5-trimethyl-1H-pyrazol-4-yl)-1H-indole-3-carbaldehyde (140 mg, 0.40 mmol) and methylamine (2.0 M in THF, 3 mL) was heated to 45° C. for 5 days, and then 50° C. for 5 days in a sealed tube. Solvent removed and residue treated with 40% aqueous methylamine (3 mL) and resulting mixture stirred in a sealed pressure tube at 60° C. for 3 days and 75° C. for 1 day. The mixture cooled to room temperature and treated with water (5 mL) and 6 M aqueous HCl until pH˜2 and stirred for 90 minutes. The mixture extracted with EtOAc (3×30 mL). Aqueous layer made basic (pH˜9) using saturated aqueous Na₂CO₃ and extracted with EtOAc (3×50 mL). EtOAc extracts of the basic aqueous layer combined, washed with saturated aqueous NaCl (25 mL), dried over Na₂SO₄, and concentrated. Yield: 85%. MS (m/z): 341.2 (MH⁺).

### Preparation of (Z)-1-(2-((5-methoxy-1-(2-(methylamino)ethyl)-2-(1,3,5-trimethyl-1H-pyrazol-4-yl)-1H-indol-3-yl)methylene)-3-oxo-2,3-dihydrobenzofuran-5-yl)-3-methylurea

Concentrated aqueous HCl (6 drops) was added to a stirred mixture of 5-methoxy-1-(2-(methylamino)ethyl)-2-(1,3,5-trimethyl-1H-pyrazol-4-yl)-1H-indole-3-carbaldehyde (104 mg, 0.31 mmol) and 1-methyl-3-(3-oxo-2,3-dihydrobenzofuran-5-yl)urea (68 mg, 0.33 mmol) in EtOH (1.6 mL). Resulting mixture stirred at 60° C. for 2 hours. Additional 1-methyl-3-(3-oxo-2,3-dihydrobenzofuran-5-yl)urea (35 mg, 0.17 mmol) added and mixture stirred at 60° C. for 90 minutes and then overnight at 40° C. The mixture cooled to room temperature, poured into water (50 mL), stirred for 30 minutes, and then filtered through Celite™. Filtrate made basic using saturated aqueous Na₂CO₃ and then concentrated. Resulting residue taken up in EtOH and then filtered. Filtrate concentrated and residue purified by preparative HPLC. Yield: 27%. MS (m/z): 529.3 (MH⁺).

### (Z)-1-(2-((1-(2-(Dimethylamino)ethyl)-5-methoxy-2-(1,3,5-trimethyl-1H-pyrazol-4-yl)-1H-indol-3-yl)methylene)-3-oxo-2,3-dihydrobenzofuran-5-yl)-3-methyl urea MS (m/z): 543.3 (M H⁺)

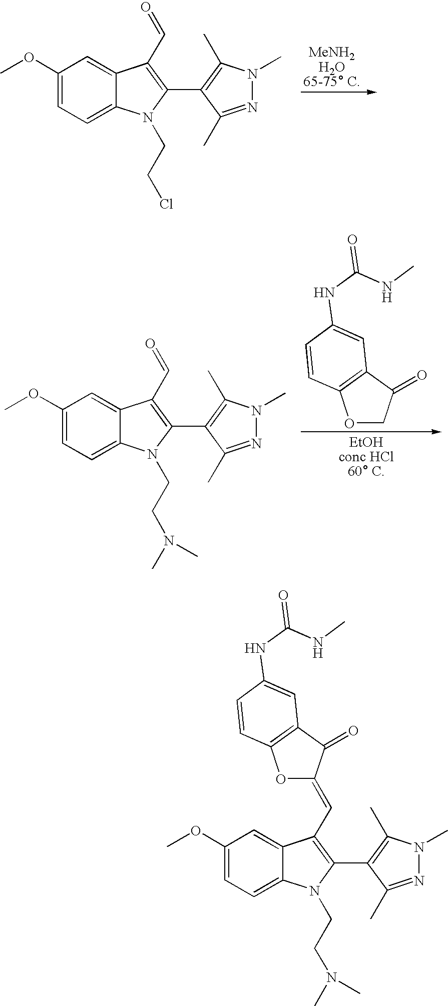

### Preparation of 1-(2-(dimethylamino)ethyl)-5-methoxy-2-(1,3,5-trimethyl-1H-pyrazol-4-yl)-1H-indole-3-carbaldehyde

A mixture of 1-(2-chloroethyl)-5-methoxy-2-(1,3,5-trimethyl-1H-pyrazol-4-yl)-1H-indole-3-carbaldehyde (176 mg, 0.51 mmol) and dimethylamine (40% in water, 2.5 mL) was stirred in a sealed pressure tube at 65° C. overnight, then 75° C. for 6 hours. The mixture cooled to room temperature and excess dimethylamine removed using a stream of N₂. The mixture acidified to pH˜2 with 3 M aqueous HCl and diluted with water (25 mL) and extracted with EtOAc (2×25 mL). Aqueous layer made basic (pH 8-9) using saturated aqueous Na₂CO₃ and extracted with EtOAc (3×40 mL). EtOAc extracts of the basic aqueous layer combined, washed with saturated aqueous NaCl (25 mL), dried over Na₂SO₄, and concentrated. Yield: 70%. MS (m/z): 355.2 (MH⁺).

### Preparation of (Z)-1-(2-((1-(2-(dimethylamino)ethyl)-5-methoxy-2-(1,3,5-trimethyl-1H-pyrazol-4-yl)-1H-indol-3-yl)methylene)-3-oxo-2,3-dihydrobenzofuran-5-yl)-3-methylurea

Concentrated aqueous HCl (6 drops) was added to a stirred mixture of 1-(2-(dimethylamino)ethyl)-5-methoxy-2-(1,3,5-trimethyl-1H-pyrazol-4-yl)-1H-indole-3-carbaldehyde (60 mg, 0.17 mmol) and 1-methyl-3-(3-oxo-2,3-dihydrobenzofuran-5-yl)urea (41 mg, 0.20 mmol) in EtOH (1.5 mL). Resulting mixture stirred at 60° C. for 4 hours. The mixture cooled to room temperature and neutralized using saturated aqueous Na₂CO₃. The mixture sat overnight at room temperature and then filtered. Filtrate concentrated and residue dissolved in MeOH and the mixture filtered. Filtrate concentrated and then purified by preparative HPLC. Yield: 32%. MS (m/z): 543.3 (MH⁺).

### (Z)-1-(2-((5-(2-Methoxyethoxy)-2-(1,3,5-trimethyl-1H-pyrazol-4-yl)-1H-indol-3-yl)methylene)-3-oxo-2,3-dihydrobenzofuran-5-yl)-3-methylurea MS (m/z): 516.2 (MH⁺)

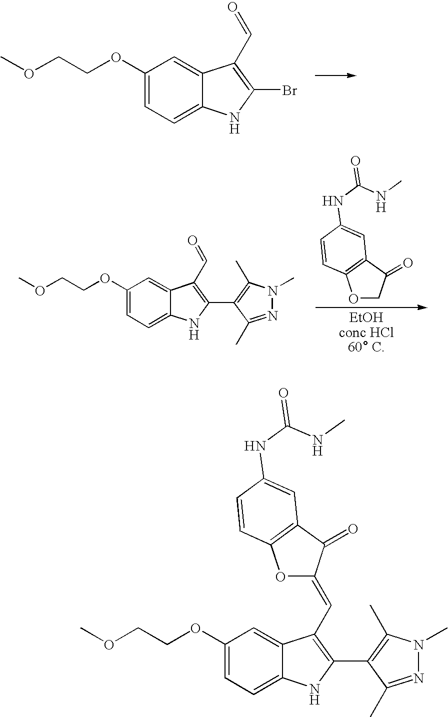

### Preparation of 2-bromo-5-(2-methoxyethoxy)-1H-indole-3-carbaldehyde

Prepared via Gassman oxindole-Vilsmeier-Haack reactions using 4-(2-methoxyethoxy)aniline. Purified by silica gel chromatography (eluent: 50% EtOAc-hexanes to 50% EtOAc-CH₂Cl₂ gradient). MS (m/z): 296.1 (MH−).

### Preparation of 5-(2-methoxyethoxy)-2-(1,3,5-trimethyl-1H-pyrazol-4-yl)-1H-indole-3-carbaldehyde

Preparation via the Suzuki coupling method using 2-bromo-5-(2-methoxyethoxy)-1H-indole-3-carbaldehyde. Purified by silica gel chromatography (eluent: 0-5% MeOH-EtOAc gradient). Yield 48%. MS (m/z): 328.2 (MH⁺).

### Preparation of (Z)-1-(2-((5-(2-methoxyethoxy)-2-(1,3,5-trimethyl-1H-pyrazol-4-yl)-1H-indol-3-yl)methylene)-3-oxo-2,3-dihydrobenzofuran-5-yl)-3-methylurea

Concentrated aqueous HCl (6 drops) was added to a stirred mixture of 5-(2-methoxyethoxy)-2-(1,3,5-trimethyl-1H-pyrazol-4-yl)-1H-indole-3-carbaldehyde (89 mg, 0.27 mmol) and 1-methyl-3-(3-oxo-2,3-dihydrobenzofuran-5-yl)urea (66 mg, 0.32 mmol) in EtOH (1.5 mL). Resulting mixture stirred at 60° C. for 4 hours. The mixture cooled to room temperature, diluted with EtOAc (3 mL) and filtered. Filtrate treated with saturated aqueous Na₂CO₃ (5 mL), stirred for 5 minutes, and then decanted into EtOH (50 mL). The mixture filtered and concentrated and residue purified by preparative HPLC. Yield: 35%. MS (m/z): 516.2 (MH⁺).

### (Z)-1-(2-((6-Fluoro-5,7-dimethoxy-2-(1,3,5-trimethyl-1H-pyrazol-4-yl)-1H-indol-3-yl)methylene)-3-oxo-2,3-dihydrobenzofuran-5-yl)-3-methylurea MS (m/z): 520.2 (MH⁺)

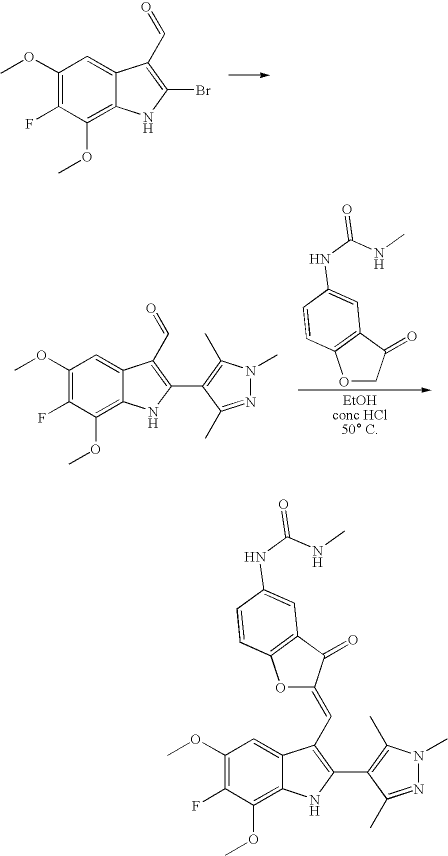

### Preparation of 2-bromo-6-fluoro-5,7-dimethoxy-1H-indole-3-carbaldehyde

Prepared via Gassman oxindole-Vilsmeier-Haack reactions using 3-fluoro-2,4-dimethoxyaniline. MS (m/z): 302.0 (MH⁺).

### Preparation of 6-fluoro-5,7-dimethoxy-2-(1,3,5-trimethyl-1H-pyrazol-4-yl)-1H-indole-3-carbaldehyde

Preparation via the Suzuki coupling method using 2-bromo-6-fluoro-5,7-dimethoxy-1H-indole-3-carbaldehyde. MS (m/z): 332.1 (MH⁺).

### Preparation of (Z)-1-(2-((6-fluoro-5,7-dimethoxy-2-(1,3,5-trimethyl-1H-pyrazol-4-yl)-1H-indol-3-yl)methylene)-3-oxo-2,3-dihydrobenzofuran-5-yl)-3-methylurea

Concentrated aqueous HCl (3 drops) was added to a stirred mixture of 6-fluoro-5,7-dimethoxy-2-(1,3,5-trimethyl-1H-pyrazol-4-yl)-1H-indole-3-carbaldehyde (116 mg, 0.35 mmol) and 1-methyl-3-(3-oxo-2,3-dihydrobenzofuran-5-yl)urea (87 mg, 0.42 mmol) in EtOH (1.5 mL). The mixture stirred at 50° C. for 2 hours. Additional concentrated aqueous HCl added (3 drops) and mixture stirred overnight at 50° C. The mixture cooled to room temperature, diluted with EtOAc (2 mL) and suction filtered through sintered glass. Filtrate treated with saturated aqueous Na₂CO₃ until pH˜8-9 and mixture heated to 60° C. for 10 minutes, then cooled to room temperature. EtOH added (5 mL) and the red solution was collected and concentrated. Residue purified by preparative HPLC. Yield: 21%. MS (m/z): 520.2 (MH⁺).

### (Z)-1-(2-((6,7-Difluoro-5-methoxy-2-(1,3,5-trimethyl-1H-pyrazol-4-yl)-1H-indol-3-yl)methylene)-3-oxo-2,3-dihydrobenzofuran-5-yl)-3-methylurea MS (m/z): 508.2 (MH⁺)

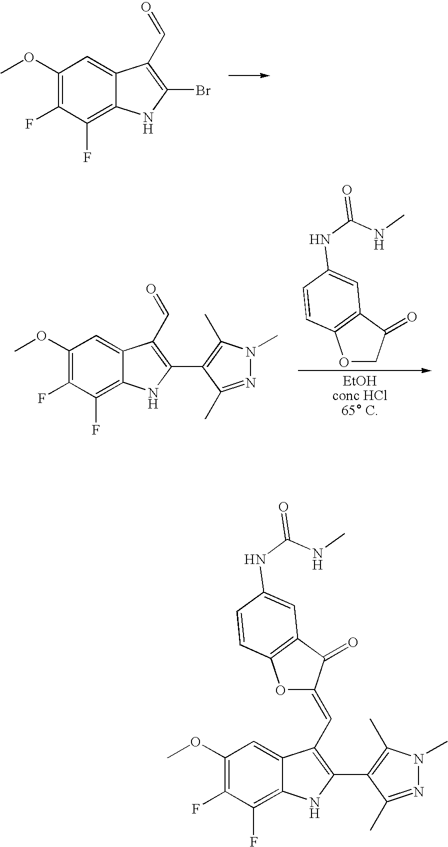

### Preparation of 2-bromo-6,7-difluoro-5-methoxy-1H-indole-3-carbaldehyde

Prepared via Gassman oxindole-Vilsmeier-Haack reactions using 2,3-difluoro-4-methoxyaniline. MS (m/z): 288.2 (MH−).

### Preparation of 6,7-difluoro-5-methoxy-2-(1,3,5-trimethyl-1H-pyrazol-4-yl)-1H-indole-3-carbaldehyde

Preparation via the Suzuki coupling method using 2-bromo-6,7-difluoro-5-methoxy-1H-indole-3-carbaldehyde. MS (m/z): 320.3 (MH⁺).

### Preparation of (Z)-1-(2-((6,7-difluoro-5-methoxy-2-(1,3,5-trimethyl-1H-pyrazol-4-yl)-1H-indol-3-yl)methylene)-3-oxo-2,3-dihydrobenzofuran-5-yl)-3-methylurea

Concentrated aqueous HCl (6 drops) was added to a stirred mixture of 6,7-difluoro-5-methoxy-2-(1,3,5-trimethyl-1H-pyrazol-4-yl)-1H-indole-3-carbaldehyde (89 mg, 0.28 mmol) and 1-methyl-3-(3-oxo-2,3-dihydrobenzofuran-5-yl)urea (52 mg, 0.25 mmol) in EtOH (2 mL). Resulting mixture stirred at 65° C. for 5 hours, and then sat overnight at room temperature. The mixture treated with EtOAc (2 mL) and filtered through sintered glass. Solid washed with 50% EtOH-EtOAc (3×2 mL) to give a yellow-orange solid that was collected and dried in vacuo. Yield: 51%. MS (m/z): 508.2 (MH⁺).

### (Z)-1-(2-((5-Methoxy-7-(trifluoromethyl)-2-(1,3,5-trimethyl-1H-pyrazol-4-yl)-1H-indol-3-yl)methylene)-3-oxo-2,3-dihydrobenzofuran-5-yl)-3-methyl urea MS (m/z): 540.2 (M H⁺)

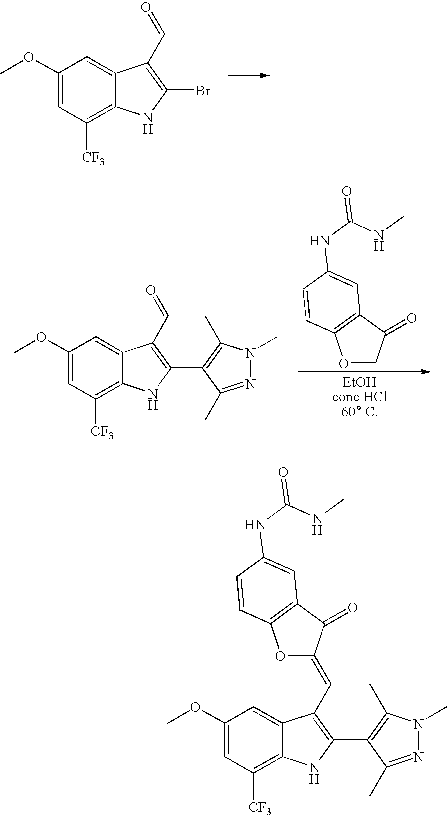

### Preparation of 2-bromo-5-methoxy-7-(trifluoromethyl)-1H-indole-3-carbaldehyde

Prepared via Gassman oxindole-Vilsmeier-Haack reactions using 4-methoxy-2-(trifluoromethyl)aniline. MS (m/z): 320.2 (MH−).

### Preparation of 5-methoxy-7-(trifluoromethyl)-2-(1,3,5-trimethyl-1H-pyrazol-4-yl)-1H-indole-3-carbaldehyde

Preparation via the Suzuki coupling method using 2-bromo-5-methoxy-7-(trifluoromethyl)-1H-indole-3-carbaldehyde. MS (m/z): 352.3 (MH⁺).

### Preparation of (Z)-1-(2-((5-methoxy-7-(trifluoromethyl)-2-(1,3,5-trimethyl-1H-pyrazol-4-yl)-1H-indol-3-yl)methylene)-3-oxo-2,3-dihydrobenzofuran-5-yl)-3-methylurea

Concentrated aqueous HCl (6 drops) was added to a stirred mixture of 5-methoxy-7-(trifluoromethyl)-2-(1,3,5-trimethyl-1H-pyrazol-4-yl)-1H-indole-3-carbaldehyde (96 mg, 0.27 mmol) and 1-methyl-3-(3-oxo-2,3-dihydrobenzofuran-5-yl)urea (56 mg, 0.27 mmol) in EtOH (2 mL). Resulting mixture stirred at 60° C. for 5 hours, and then 45° C. overnight. The mixture cooled to room temperature and EtOAc added (2 mL). The mixture suction filtered through sintered glass and resulting solid washed with 20% EtOH-EtOAc (3 mL). The tan solid dried in vacuo. Yield: 34%. MS (m/z): 540.2 (MH⁺).

### (Z)-1-(2-((5-methoxy-7-methyl-2-(1,3,5-trimethyl-1H-pyrazol-4-yl)-1H-indol-3-yl)methylene)-3-oxo-2,3-dihydrobenzofuran-5-yl)-3-methylurea MS (m/z): 486.2 (MH⁺)

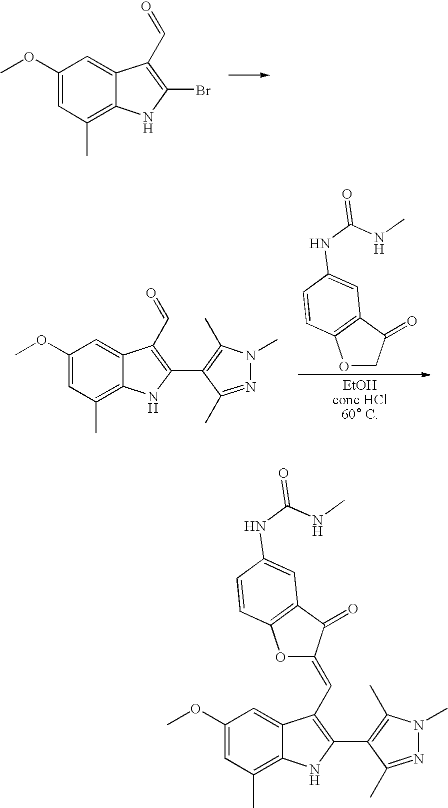

### Preparation of 2-bromo-5-methoxy-7-methyl-1H-indole-3-carbaldehyde

Prepared via Gassman oxindole-Vilsmeier-Haack reactions using 4-methoxy-2-methylaniline. MS (m/z): 268.2 (MH⁺).

### Preparation of 5-methoxy-7-methyl-2-(1,3,5-trimethyl-1H-pyrazol-4-yl)-1H-indole-3-carbaldehyde

Preparation via the Suzuki coupling method using 2-bromo-5-methoxy-7-methyl-1H-indole-3-carbaldehyde. MS (m/z): 298.3 (MH⁺).

### Preparation of (Z)-1-(2-((5-methoxy-7-methyl-2-(1,3,5-trimethyl-1H-pyrazol-4-yl)-1H-indol-3-yl)methylene)-3-oxo-2,3-dihydrobenzofuran-5-yl)-3-methylurea

Concentrated aqueous HCl (6 drops) was added to a stirred mixture of 5-methoxy-7-methyl-2-(1,3,5-trimethyl-1H-pyrazol-4-yl)-1H-indole-3-carbaldehyde (85 mg, 0.29 mmol) and 1-methyl-3-(3-oxo-2,3-dihydrobenzofuran-5-yl)urea (59 mg, 0.29 mmol) in EtOH (2 mL). The mixture stirred at 60° C. for 5 hours, and then 45° C. overnight. The mixture cooled to room temperature, diluted with EtOH (3 mL), and treated with saturated aqueous Na₂CO₃ (3 mL). Resulting mixture sonicated for 2-3 minutes, filtered, and filtrate concentrated. Residue treated with 25% EtOH-EtOAc and filtered. Filtrate sat overnight. An orange solid precipitated from the filtrate that was collected and dried in vacuo. Yield: 14%. MS (m/z): 486.2 (MH⁺).

### (Z)-1-(2-((2-(3,5-Dimethyl-1H-pyrazol-4-yl)-7-fluoro-5-methoxy-1H-indol-3-yl)methylene)-3-oxo-2,3-dihydrobenzofuran-5-yl)-3-methylurea MS (m/z): 476.2 (MH⁺)

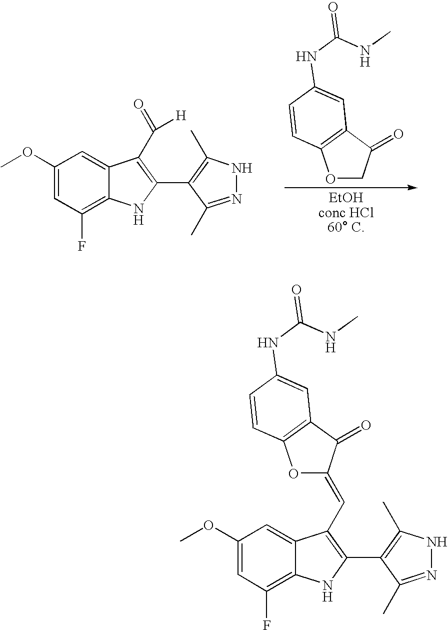

Preparation via the Suzuki coupling method using 2-bromo-7-fluoro-5-methoxy-1H-indole-3-carbaldehyde. MS (m/z): 288.3 (MH⁺).

### (Z)-1-(2-((7-Fluoro-2-(1-isobutyl-1H-pyrazol-4-yl)-5-methoxy-1H-indol-3-yl)methylene)-3-oxo-2,3-dihydrobenzofuran-5-yl)-3-methylurea MS (m/z): 504.2 (MH⁺)

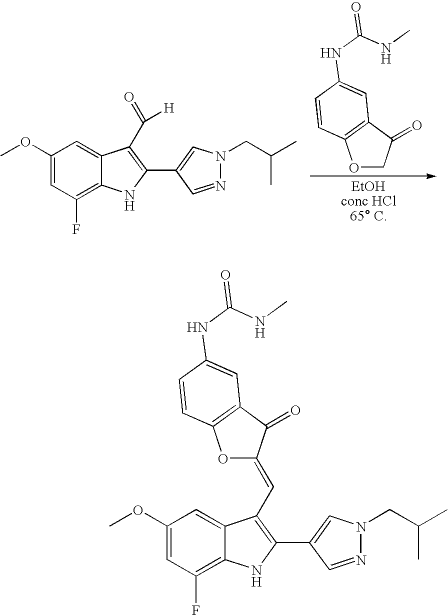

### Preparation of 7-fluoro-2-(1-isobutyl-1H-pyrazol-4-yl)-5-methoxy-1H-indole-3-carbaldehyde

Suzuki coupling method using 2-bromo-7-fluoro-5-methoxy-1H-indole-3-carbaldehyde. MS (m/z): 316.3 (MH⁺).

### Preparation of (Z)-1-(2-((7-fluoro-2-(1-isobutyl-1H-pyrazol-4-yl)-5-methoxy-1H-indol-3-yl)methylene)-3-oxo-2,3-dihydrobenzofuran-5-yl)-3-methylurea

Concentrated aqueous HCl (6 drops) was added to a stirred mixture of 7-fluoro-2-(1-isobutyl-1H-pyrazol-4-yl)-5-methoxy-1H-indole-3-carbaldehyde (80 mg, 0.25 mmol) and 1-methyl-3-(3-oxo-2,3-dihydrobenzofuran-5-yl)urea (47 mg, 0.23 mmol) in EtOH (2 mL). Resulting mixture stirred at 65° C. for 5 hours, and then 45° C. overnight. The mixture cooled to room temperature, diluted with EtOH (5 mL), and treated with saturated aqueous Na₂CO₃ (3 mL). Organic portion collected and concentrated. Resulting residue taken up in MeOH (5 mL) and filtered. Filtrate triturated with EtOAc until solid material was observed. The mixture let sit overnight. Mother liquor was collected from the solid and concentrated and resulting material purified by preparative HPLC. Yield: 46%. MS (m/z): 504.2 (MH⁺).

### (Z)-1-(2-((7-Fluoro-5-methoxy-2-(1-methyl-1H-pyrazol-4-yl)-1H-indol-3-yl)methylene)-3-oxo-2,3-dihydrobenzofuran-5-yl)-3-methylurea MS (m/z): 462.2 (MH⁺)

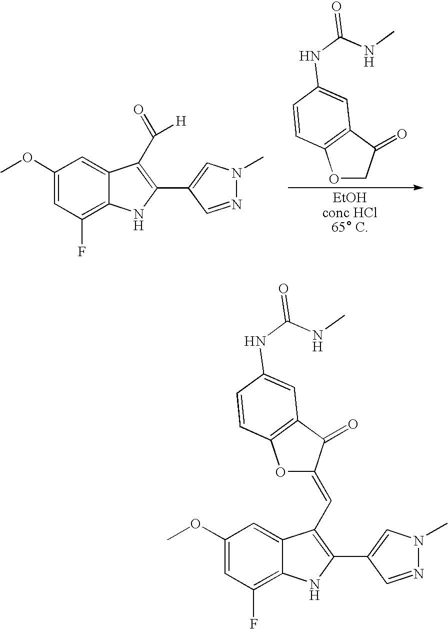

### Preparation of 7-fluoro-5-methoxy-2-(1-methyl-1H-pyrazol-4-yl)-1H-indole-3-carbaldehyde

Suzuki coupling method using 2-bromo-7-fluoro-5-methoxy-1H-indole-3-carbaldehyde. MS (m/z): 274.2 (MH⁺).

### Preparation of (Z)-1-(2-((7-fluoro-5-methoxy-2-(1-methyl-1H-pyrazol-4-yl)-1H-indol-3-yl)methylene)-3-oxo-2,3-dihydrobenzofuran-5-yl)-3-methylurea

Concentrated aqueous HCl (6 drops) was added to a stirred mixture of 7-fluoro-5-methoxy-2-(1-methyl-1H-pyrazol-4-yl)-1H-indole-3-carbaldehyde (100 mg, 0.37 mmol) and 1-methyl-3-(3-oxo-2,3-dihydrobenzofuran-5-yl)urea (68 mg, 0.33 mmol) in EtOH (2.5 mL). Resulting mixture stirred at 65° C. for 5 hours, and then 45° C. overnight. The mixture cooled to room temperature, diluted with EtOAc (2 mL) and filtered through sintered glass. Dark brown solid treated with DMSO (4 mL) and filtered through sintered glass. DMSO solution poured into water (20 mL) and resulting orange solid filtered. Orange solid washed with EtOH (10 mL) and filtered. Solid dried in vacuo. Yield: 32%. MS (m/z): 462.2 (MH⁺).

### N-[2-(Dimethylamino)ethyl]-4-({[(2Z)-2-{[7-fluoro-5-methoxy-2-(1,3,5-trimethyl-1H-pyrazol-4-yl)-1 indol-3-yl]methylidene}-3-oxo-2,3-dihydro-1-benzofuran-5-yl]carbamoyl}amino)-N-methylbenzamide MS (m/z): 680.2 (MH⁺)

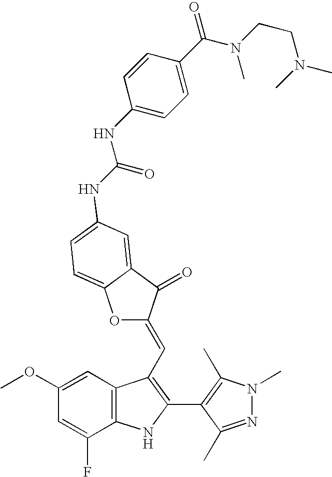

### Synthetic Scheme:

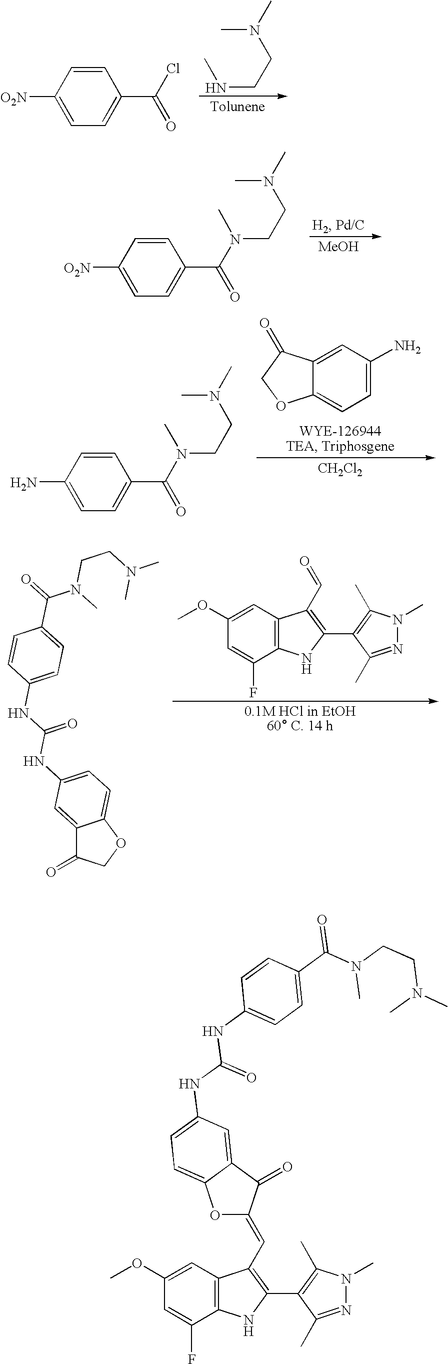

### N-(2-(Dimethylamino)ethyl)-N-methyl-4-nitrobenzamide

Into a solution of 4-nitrobenzoyl chloride (12 g, 64.7 mmol) in toluene (200 ml) was added in drops N1,N1,N2-trimethylethane-1,2-diamine (10.09 mL, 78 mmol). The reaction mixture was vigorously stirred at room temperature for 14 hours, then suction filtered. The solid was partitioned between ethyl acetate and saturated NaHCO₃ aqueous solution. The organic layer was washed with saturated NaCl aqueous solution, dried over MgSO₄, suction filtered, concentrated and dried further in vacuo to give N-(2-(dimethylamino)ethyl)-N-methyl-4-nitrobenzamide (9.2 g, 36.6 mmol, 56.6%) as a white solid. MS (m/z): 252.2 (MH⁺)

### 4-Amino-N-(2-(dimethylamino)ethyl)-N-methylbenzamide

Into an solution of N-(2-(dimethylamino)ethyl)-N-methyl-4-nitrobenzamide (4 g, 15.92 mmol) in methanol (50 ml) was added Pd—C 10% (1 g, 0.940 mmol). The reaction flask was sealed with a rubber septa and a 2 L balloon of hydrogen gas was inserted. The reaction mixture was stirred under the hydrogen balloon pressure at room temperature for 14 hours. The resulting reaction mixture was suction filtered through a Celite™ bed. The filtrate was concentrated and dried further in vacuo to give 3.5 g of the desired product 4-amino-N-(2-(dimethylamino)ethyl)-N-methylbenzamide (3.5 g, 15.82 mmol, 99%) as a colorless gel. MS (m/z): 222.2 (MH⁺)

### N-[2-(Dimethylamino)ethyl]-N-methyl-4-{[(3-oxo-2,3-dihydro-1-benzofuran-5-yl)carbamoyl]amino}benzamide TFA salt

Into as solution of 5-aminobenzofuran-3(2H)-one (1 g, 6.70 mmol) in dichloromethane (50 ml) was added triethylamine (0.890 mL, 6.70 mmol) followed by an addition of triphosgene (0.657 g, 2.213 mmol) in dichloromethane solution (10 ml). The mixture was stirred for 1 hour and 4-amino-N-(2-(dimethylamino)ethyl)-N-methylbenzamide (1.484 g, 6.70 mmol) in dichloromethane (20 ml) was added. The reaction mixture was stirred at room temperature for 14 hours, then diluted with methanol and suction filtered. The filtrate was concentrated, re-dissolved with DMSO (10 ml) and suction filtered. The DMSO filtrate was purified by HPLC to give the desired product N-[2-(dimethylamino)ethyl]-N-methyl-4-{[(3-oxo-2,3-dihydro-1-benzofuran-5-yl)carbamoyl]amino}benzamide TFA salt (1.28 g, 2.508 mmol, 37.4%) as a light yellow solid. MS (m/z): 397.2 (MH⁺)

### (Z)-N-(2-(Dimethylamino)ethyl)-4-(3-(2-((7-fluoro-5-methoxy-2-(1,3,5-trimethyl-1H-pyrazol-4-yl)-1H-indol-3-yl)methylene)-3-oxo-2,3-dihydrobenzofuran-5-yl)ureido)-N-methyl benzamide TFA salt

A mixture of N-(2-(dimethylamino)ethyl)-N-methyl-4-(3-(3-oxo-2,3-dihydrobenzofuran-5-yl)ureido)benzamide TFA salt (2.4 g, 4.70 mmol) and 7-fluoro-5-methoxy-2-(1,3,5-trimethyl-1H-pyrazol-4-yl)-1H-indole-3-carbaldehyde (1.417 g, 4.70 mmol) in 0.1M HCl solution in ethanol (100 ml) was stirred at 60° C. for 18 hours, then concentrated. The residue was purified by HPLC (0.1% TFA) to give N-[2-(dimethylamino)ethyl]-4-({[(2Z)-2-{[7-fluoro-5-methoxy-2-(1,3,5-trimethyl-1H-pyrazol-4-yl)-1H-indol-3-yl]methylidene}-3-oxo-2,3-dihydro-1-benzofuran-5-yl]carbamoyl}amino)-N-methylbenzamide TFA salt (1.58 g, 1.931 mmol, 41.1%) as an orange solid. MS (m/z): 680.2 (MH⁺)

The following compounds were synthesized using the procedure above.

### N-[2-(Dimethylamino)ethyl]-4-({[(2Z)-2-{[7-fluoro-5-methoxy-2-(1,3,5-trimethyl-1H-pyrazol-4-yl)-1 indol-3-yl]methylidene}-3-oxo-2,3-dihydro-1-benzofuran-5-yl]carbamoyl}amino)benzamide MS (m/z): 666.4 (MH⁺)

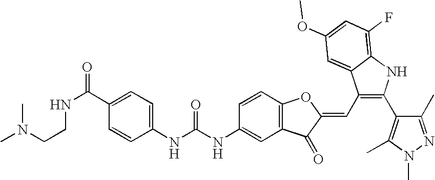

### 1-(4-{[3-(Dimethylamino)pyrrolidin-1-yl]carbonyl}phenyl)-3-[(2Z)-2-{[7-fluoro-5-methoxy-2-(1,3,5-trimethyl-1H-pyrazol-4-yl)-1H-indol-3-yl]methylidene}-3-oxo-2,3-dihydro-1-benzofuran-5-yl]urea MS (m/z): 692.4 (MH⁺)

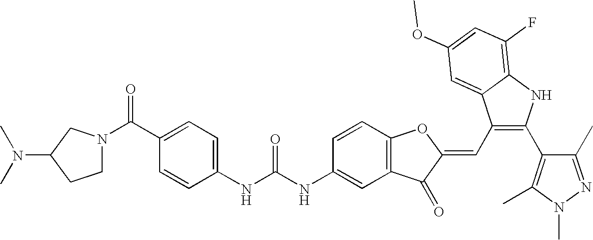

### 1-{4-[(3,4-Dimethylpiperazin-1-yl)carbonyl]phenyl}-3-[(2Z)-2-{[7-fluoro-5-methoxy-2-(1,3,5-trimethyl-1H-pyrazol-4-yl)-1H-indol-3-yl]methylidene}-3-oxo-2,3-dihydro-1-benzofuran-5-yl]urea MS (m/z): 692.3 (MH⁺)

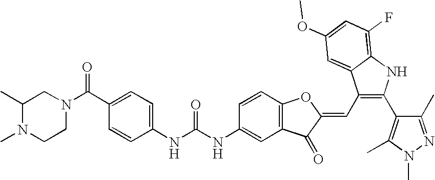

### 1-(4-{[4-(Dimethylamino)piperidin-1-yl]carbonyl}phenyl)-3-[(2Z)-2-{[7-fluoro-5-methoxy-2-(1,3,5-trimethyl-1H-pyrazol-4-yl)-1H-indol-3-yl]methylidene}-3-oxo-2,3-dihydro-1-benzofuran-5-yl]urea MS (m/z): 706.5 (MH⁺)

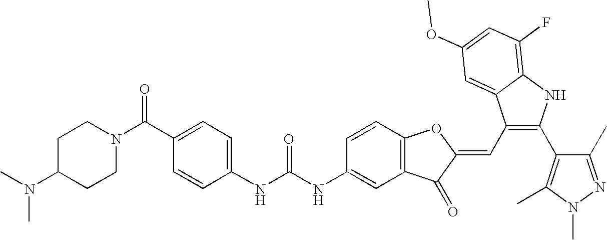

### 1-[(2Z)-2-{[7-Fluoro-5-methoxy-2-(1,3,5-trimethyl-1H-pyrazol-4-yl)-1H-indol-3-yl]methylidene}-3-oxo-2,3-dihydro-1-benzofuran-5-yl]-3-[4-(morpholin-4-ylcarbonyl)phenyl]urea MS (m/z): 665.4 (MH⁺)

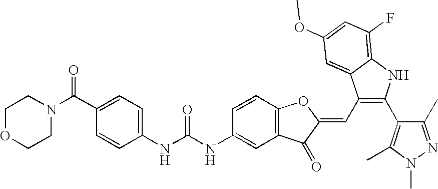

### (1-[(2Z)-2-{[7-Fluoro-5-methoxy-2-(1,3,5-trimethyl-1H-pyrazol-4-yl)-1H-indol-3-yl]methylidene}-3-oxo-2,3-dihydro-1-benzofuran-5-yl]-3-(4-{[4-(2-hydroxyethyl)piperazin-1-yl]carbonyl}phenyl)urea MS (m/z): 708.2 (MH⁺)

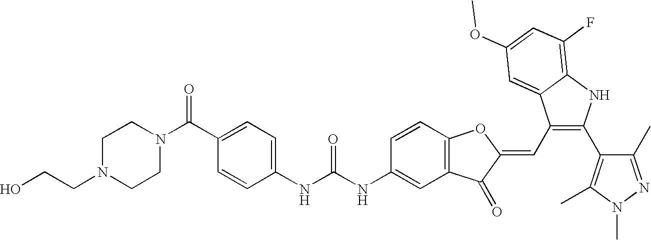

### N-[2-(Dimethylamino)ethyl]-4-({[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]carbamoyl}amino)-N-methylbenzamide MS (m/z): 662.4 (MH⁺)

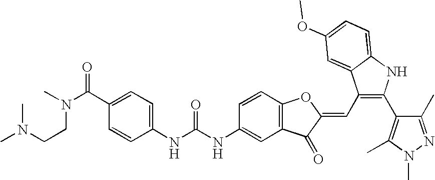

### Synthetic Scheme:

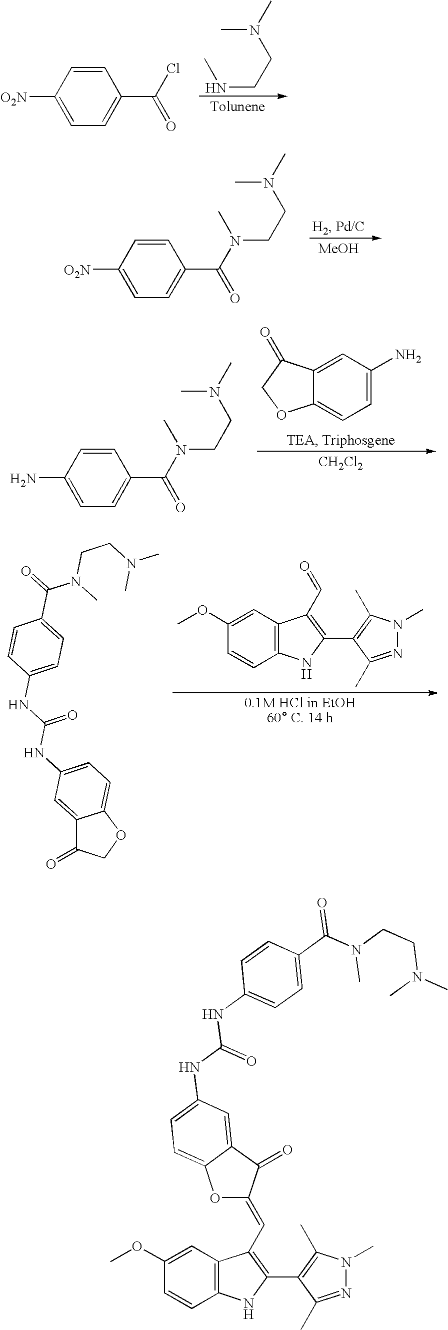

### N-[2-(Dimethylamino)ethyl]-4-({[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]carbamoyl}amino)benzamide MS (m/z): 648.3 (MH⁺)

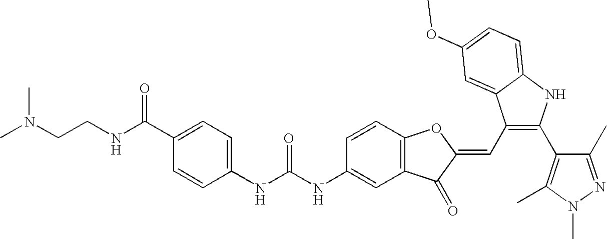

### 4-({[(2Z)-2-{[5-Methoxy-2-(1,3,5-trimethyl-1H-pyrazol-4-yl)-1H-indol-3-yl]methylidene}-3-oxo-2,3-dihydro-1-benzofuran-5-yl]carbamoyl}amino)-N,N-dimethyl benzamide MS (m/z): 605.3 (MH⁺)

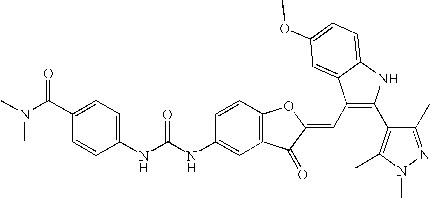

### 1-{4-[(3,4-Dimethylpiperazin-1-yl)carbonyl]phenyl}-3-[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]urea MS (m/z): 674.3 (MH⁺)

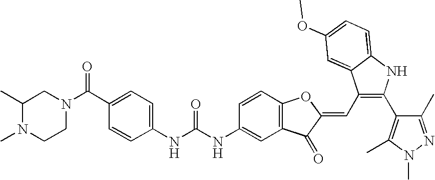

### 4-({[(2Z)-2-{[5-Methoxy-2-(1,3,5-trimethyl-1H-pyrazol-4-yl)-1H-indol-3-yl]methylidene}-3-oxo-2,3-dihydro-1-benzofuran-5-yl]carbamoyl}amino)-N-methyl-N-(2-pyrrolidin-1-ylethyl)benzamide MS (m/z): 688.3 (MH⁺)

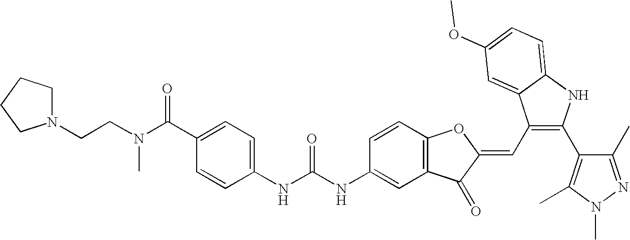

### 1-(4-{[4-(Dimethylamino)piperidin-1-yl]carbonyl}phenyl)-3-[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]urea MS (m/z): 688.5 (MH⁺)

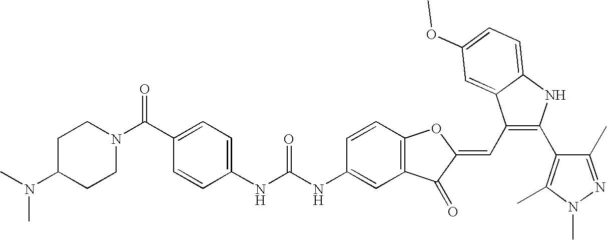

### 1-[(2Z)-2-{[5-Methoxy-2-(1,3,5-trimethyl-1H-pyrazol-4-yl)-1H-indol-3-yl]methylidene}-3-oxo-2,3-dihydro-1-benzofuran-5-yl]-3-(4-{[4-(1-methylethyl)piperazin-1-yl]carbonyl}phenyl)urea MS (m/z): 688.3 (MH⁺)

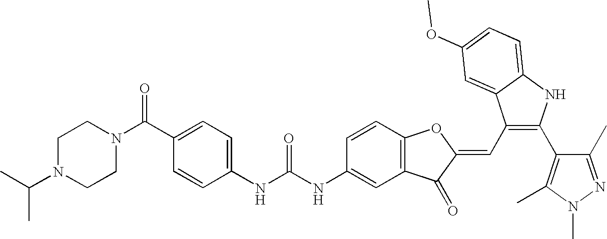

### 4-({[(2Z)-2-{[5-Methoxy-2-(1,3,5-trimethyl-1H-pyrazol-4-yl)-1H-indol-3-yl]methylidene}-3-oxo-2,3-dihydro-1-benzofuran-5-yl]carbamoyl}amino)-N-methyl-N-(1-methylpyrrolidin-3-yl)benzamide MS (m/z): 674.2 (MH⁺)

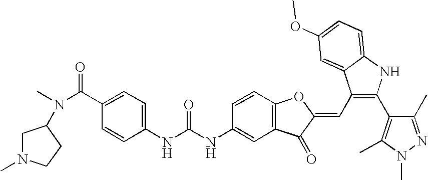

### 1-{4-[(4-Ethyl piperazin-1-yl)carbonyl]phenyl}-3-[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]urea MS (m/z): 674.2 (MH⁺)

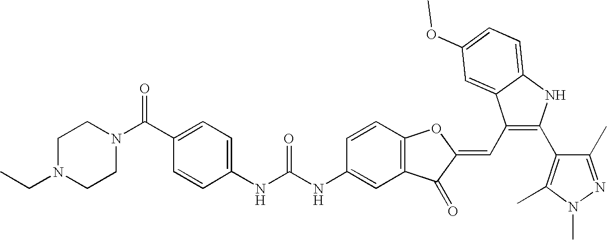

### 1-(4-{[3-(Dimethylamino)pyrrolidin-1-yl]carbonyl}phenyl)-3-[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]urea MS (m/z): 674.1 (MH⁺)

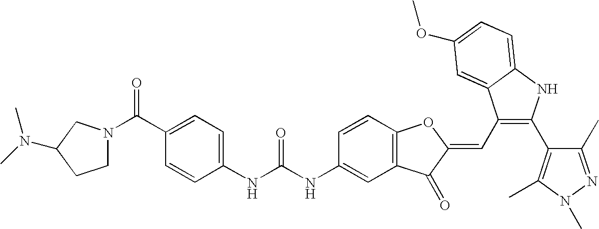

### 1-[(2Z)-2-{[5-Methoxy-2-(1,3,5-trimethyl-1H-pyrazol-4-yl)-1H-indol-3-yl]methylidene}-3-oxo-2,3-dihydro-1-benzofuran-5-yl]-3-[4-(morpholin-4-ylcarbonyl)phenyl]urea MS (m/z): 647.3 (MH⁺)

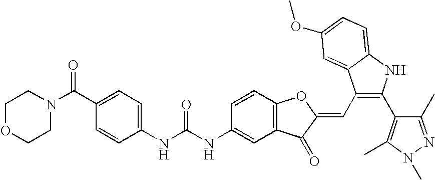

### 1-{4-[(1,1-Dioxidothiomorpholin-4-yl)carbonyl]phenyl}-3-[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]urea MS (m/z): 695.1 (MH⁺)

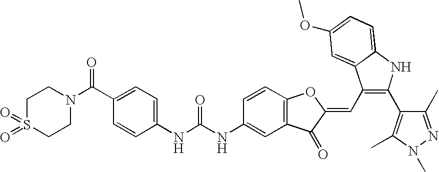

### 1-[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]-3-{4-[(4-methylpiperazin-1-yl)carbonyl]phenyl}urea MS (m/z): 660.1 (MH⁺)

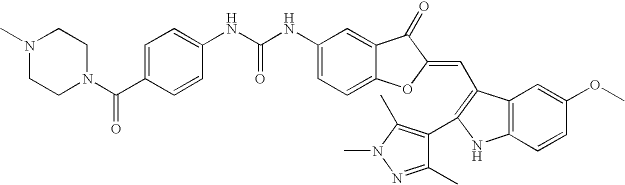

### 4-[({(2Z)-2-[(2-Cyclohexyl-5-methoxy-1H-indol-3-yl)methylidene]-3-oxo-2,3-dihydro-1-benzofuran-5-yl}carbamoyl)amino]-N-[2-(dimethylamino)ethyl]-N-methyl benzamide MS (m/z): 636.4 (MH⁺)

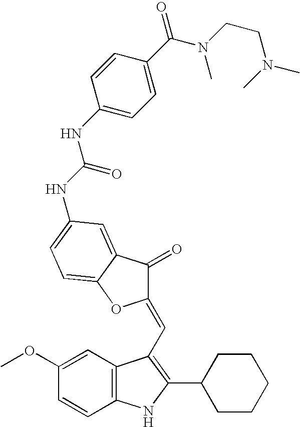

### Synthetic Scheme:

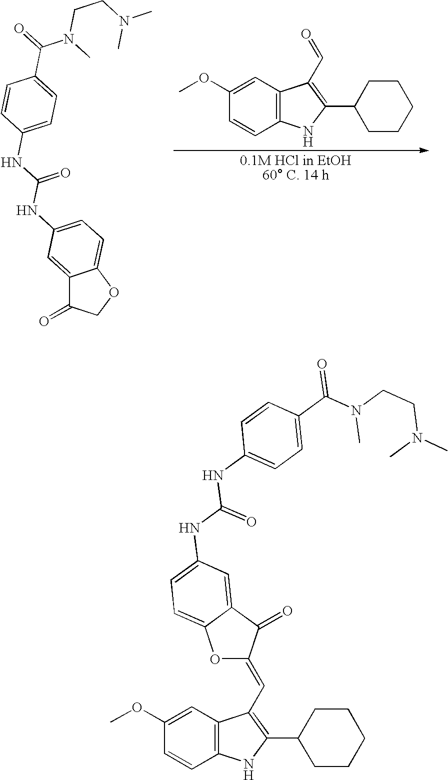

### 1-{(2Z)-2-[(2-Cyclohexyl-5-methoxy-1H-indol-3-yl)methylene]-3-oxo-2,3-dihydro-1-benzofuran-5-yl}-3-{4-[(4-methylpiperazin-1-yl)carbonyl]phenyl}urea MS (m/z): 634.3 (MH⁺)

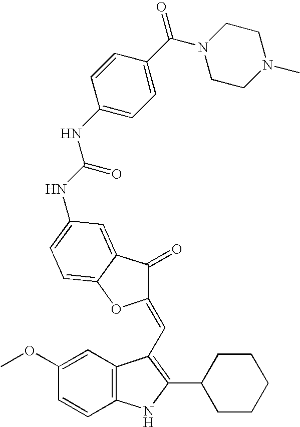

### 1-{(2Z)-2-[(2-Cyclohexyl-1-ethyl-5-methoxy-1H-indol-3-yl)methylene]-3-oxo-2,3-dihydro-1-benzofuran-5-yl}-3-{4-[(4-methylpiperazin-1-yl)carbonyl]phenyl}urea MS (m/z): 662.4 (MH⁺)

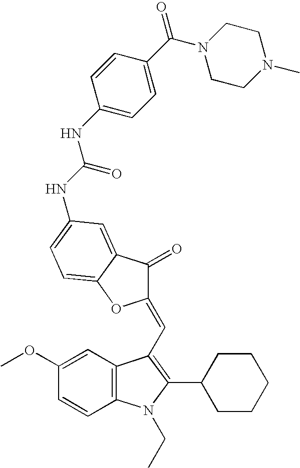

### Synthetic Scheme:

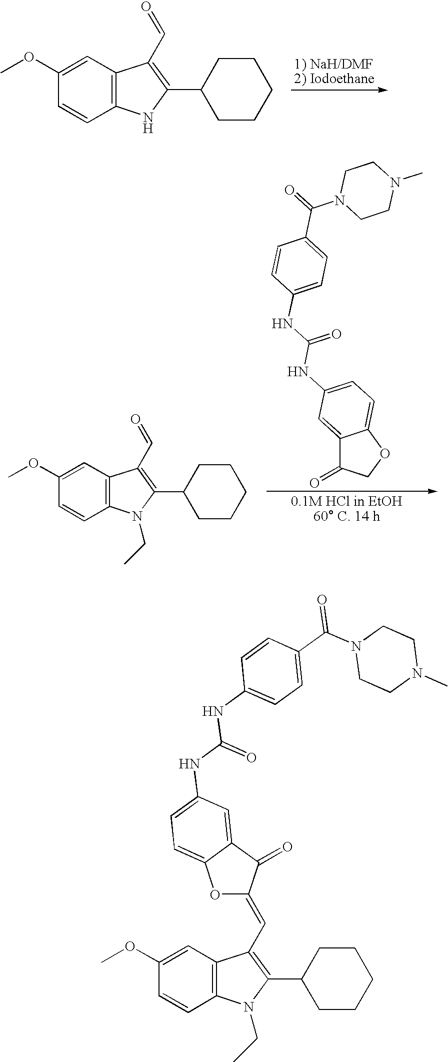

### Step 1: 2-Cyclohexyl-1-ethyl-5-methoxy-1H-indole-3-carbaldehyde

Into a solution of 2-cyclohexyl-5-methoxy-1H-indole-3-carbaldehyde (128.6 mg, 0.5 mmol) in DMF (10 mL) was added NaH (40 mg, 1.0 mmol). The mixture was stirred at room temperature for 30 minutes and iodoethane (389 mg, 2.5 mmol) was added. The reaction mixture was stirred at room temperature for 2 hours, then partitioned between water and ethyl acetate. The organic layer was washed with saturated NaCl aqueous solution, dried over MgSO₄, filtered, concentrated and chromatographed over a 40 g silica column (eluting with hexanes:ethyl acetate 1:1) to provide the desired product 2-cyclohexyl-1-ethyl-5-methoxy-1H-indole-3-carbaldehyde (107 mg, 0.35 mmol, 75%) as light yellow solid. MS (m/z): 286.2 (MH⁺)

### 1-{(2Z)-2-[(2-Cyclohexyl-5-methoxy-1H-indol-3-yl)methylene]-3-oxo-2,3-dihydro-1-benzofuran-5-yl}-3-methylurea MS (m/z): 446.2 (MH⁺)

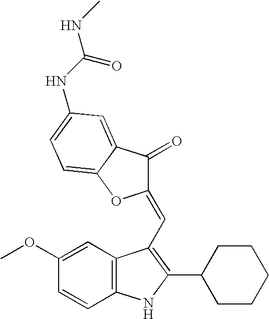

### Synthetic Scheme:

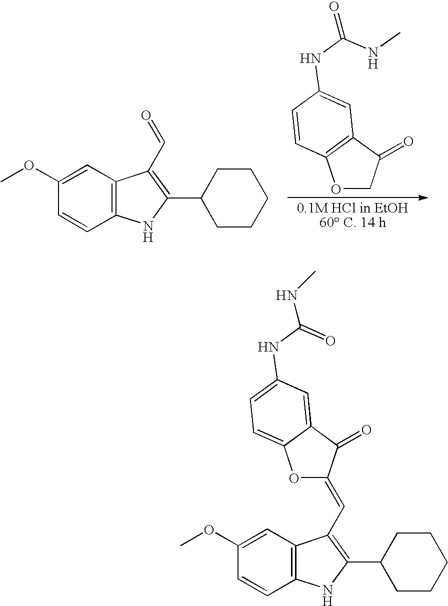

### 1-[4-(Dimethylamino)phenyl]-3-[(2Z)-2-({5-methoxy-2-methyl-1-[2-(4-methylpiperazin-1-yl)ethyl]-1H-indol-3-yl}methylidene)-3-oxo-2,3-dihydro-1-benzofuran-5-yl]urea MS (m/z): 609.4 (MH⁺)

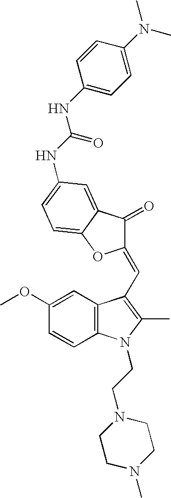

### Synthetic Scheme:

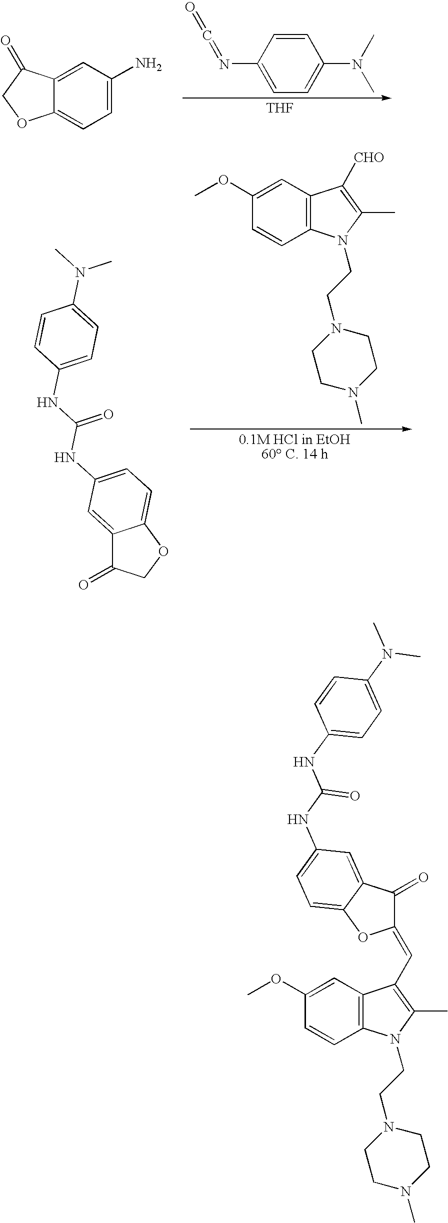

### 1-[4-(Dimethylamino)phenyl]-3-(3-oxo-2,3-dihydro-1-benzofuran-5-yl)urea

A mixture of 5-amino-1-benzofuran-3(2H)-one (280 mg, 1.88 mmol) and 4-(dimethylamino) phenyl isocyanate (304 mg, 1.88 mmol) and triethylamine (65 μL, 0.49 mmol) in THF (10 ml) was stirred at room temperature for 12 hours. The resulting reaction mixture was suction filtered and dried further in vacuo to provide 1-[4-(dimethylamino)phenyl]-3-(3-oxo-2,3-dihydro-1-benzofuran-5-yl)urea (357.5 mg, 61%) as a light yellow solid. MS (m/z): 312.2 (MH⁺)

### 1-[(2Z)-2-({5-Methoxy-2-methyl-1-[2-(4-methylpiperazin-1-yl)ethyl]-1H-indol-3-yl}methylidene)-3-oxo-2,3-dihydro-1-benzofuran-5-yl]-3-pyridin-3-yl urea MS (m/z): 567.3 (MH⁺)

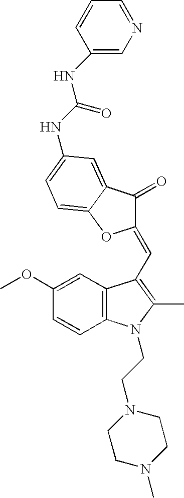

### 1-{(2Z)-2-[(2-{4-[(Dimethylamino)methyl]phenyl}-5-methoxy-1H-indol-3-yl)methylidene]-3-oxo-2,3-dihydro-1-benzofuran-5-yl}-3-methyl urea MS (m/z): 497.3 (MH⁺)

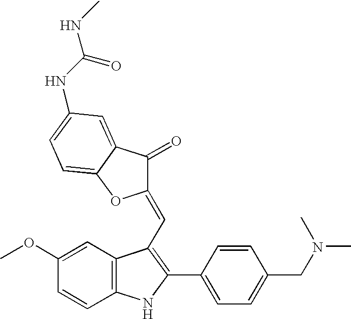

### 1-{(2Z)-2-[(2-{4-[(Dimethylamino)methyl]phenyl}-5-methoxy-1-[2-(4-methylpiperazin-1-yl)ethyl]-1H-indol-3-yl)methylidene]-3-oxo-2,3-dihydro-1-benzofuran-5-yl}-3-methyl urea MS (m/z): 312.2 (MH²⁺)

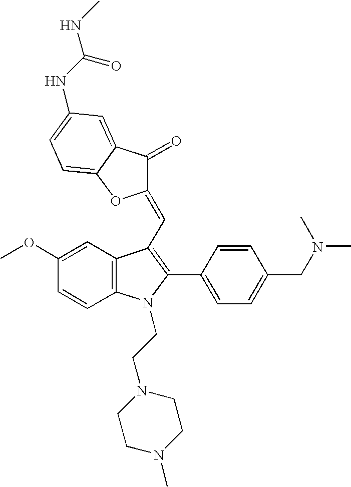

### 1-{(2Z)-2-[(2-{3-[(Dimethylamino)methyl]phenyl}-5-methoxy-1H-indol-3-yl)methylidene]-3-oxo-2,3-dihydro-1-benzofuran-5-yl}-3-methyl urea MS (m/z): 497.3 (MH⁺)

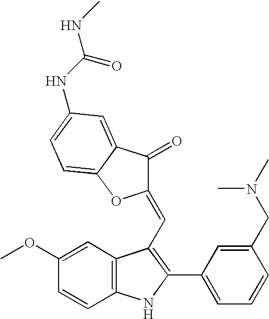

### 1-[(2Z)-2-({2-Cyclopentyl-5-methoxy-1-[2-(4-methylpiperazin-1-yl)ethyl]-1H-indol-3-yl}methylene)-3-oxo-2,3-dihydro-1-benzofuran-5-yl]-3-pyridin-3-yl urea MS (m/z): 311.2 (M2H⁺⁺)

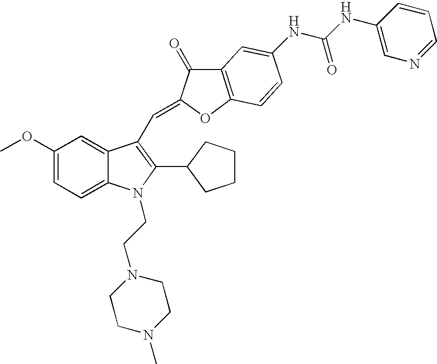

### 1-[(2Z)-2-({5-Methoxy-2-methyl-1-[2-(4-methylpiperazin-1-yl)ethyl]-1H-indol-3-yl}methylene)-3-oxo-2,3-dihydro-1-benzofuran-5-yl]-3-methylthiourea MS (m/z): 520.3 (MH⁺)

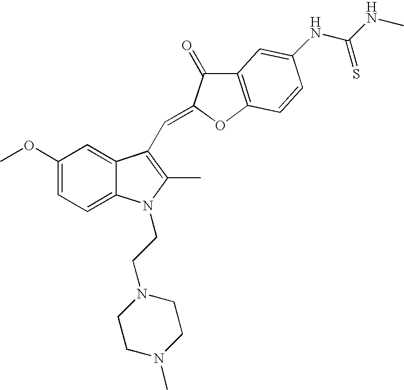

### 1-[(2Z)-2-{[7-Fluoro-5-methoxy-2-(1,3,5-trimethyl-1H-pyrazol-4-yl)-1H-indol-3-yl]methylidene}-3-oxo-2,3-dihydro-1-benzofuran-5-yl]-3-[2-(methylamino)ethyl]urea MS (m/z): 533.4 (MH⁺)

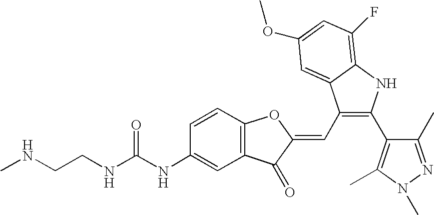

### Synthetic Scheme:

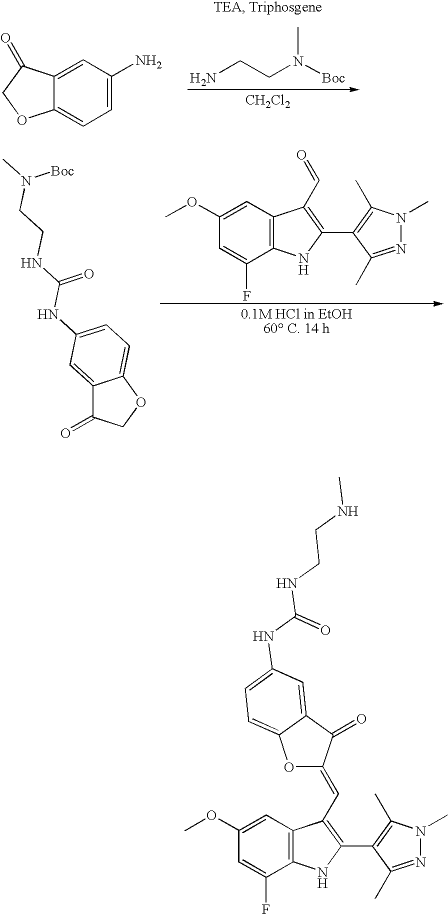

### tert-Butyl methyl(2-(3-(3-oxo-2,3-dihydrobenzofuran-5-yl) ureido)ethyl)carbamate

Into a solution of 5-aminobenzofuran-3(2H)-one (149 mg, 1 mmol) in THF (40 mL) was added triethylamine (139 μL, 1 mmol) followed by addition of triphosgene (98 mg, 0.330 mmol). The mixture was stirred at room temperature for 1 hour and tert-butyl 2-aminoethyl(methyl)carbamate (174 mg, 1.000 mmol) was added. The reaction mixture was stirred at room temperature for 12 hours, then concentrated. The residue was chromatograph over a 40 g of silica, eluting with ethyl acetate to provide tert-butyl methyl(2-(3-(3-oxo-2,3-dihydrobenzofuran-5-yl)ureido)ethyl)carbamate (148 mg, 0.424 mmol, 42.4%) as a beige solid. MS (m/z): 350.4 (MH⁺)

### 1-(2-Aminoethyl)-3-[(2Z)-2-{[7-fluoro-5-methoxy-2-(1,3,5-trimethyl-1H-pyrazol-4-yl)-1H-indol-3-yl]methylidene}-3-oxo-2,3-dihydro-1-benzofuran-5-yl]urea MS (m/z): 519.2 (MH⁺)

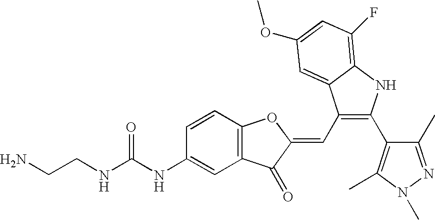

### 1-[2-(Dimethylamino)ethyl]-3-[(2Z)-2-{[7-fluoro-5-methoxy-2-(1,3,5-trimethyl-1H-pyrazol-4-yl)-1H-indol-3-yl]methylidene}-3-oxo-2,3-dihydro-1-benzofuran-5-yl]urea MS (m/z): 547.2 (MH⁺)

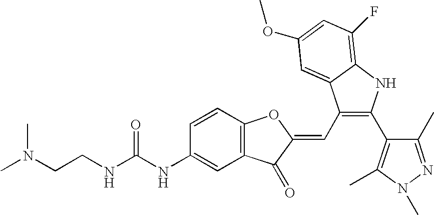

### 1-[(2Z)-2-{[7-Fluoro-5-methoxy-2-(1,3,5-trimethyl-1H-pyrazol-4-yl)-1H-indol-3-yl]methylidene}-3-oxo-2,3-dihydro-1-benzofuran-5-yl]-3-(2-pyrrolidin-1-ylethyl)urea MS (m/z): 573.6 (MH⁺)

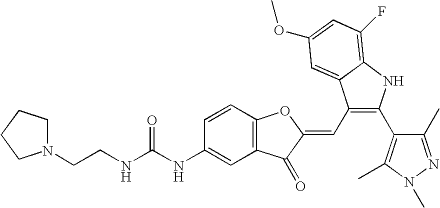

### N-[2-(Dimethylamino)ethyl]-3-({[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]carbamoyl}amino)-N-methylbenzamide MS (m/z): 662.3 (MH⁺)

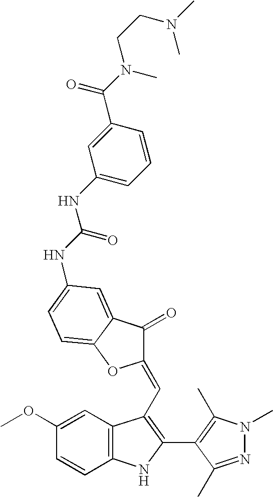

### N-[3-(Dimethylamino)propyl]-3-({[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]carbamoyl}amino)-N-methylbenzamide MS (m/z): 676.3 (MH⁺)

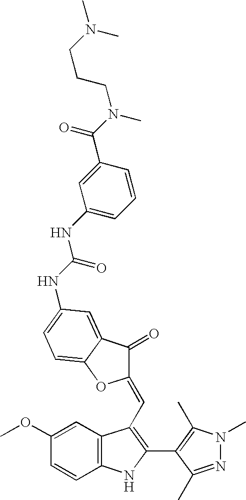

### N-[2-(Dimethylamino)ethyl]-4-({[(2Z)-2-({5-methoxy-2-methyl-1-[2-(4-methylpiperazin-1-yl)ethyl]-1H-indol-3-yl}methylene)-3-oxo-2,3-dihydro-1-benzofuran-5-yl]carbamoyl}amino)-N-methylbenzamide MS (m/z): 694.4 (MH⁺)

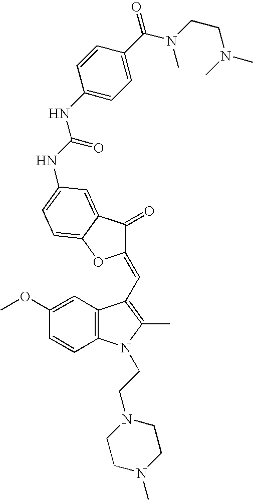

### 1-[(2Z)-2-({5-Methoxy-2-methyl-1-[2-(4-methylpiperazin-1-yl)ethyl]-1H-indol-3-yl}methylidene)-3-oxo-2,3-dihydro-1-benzofuran-5-yl]-3-[4-(morpholin-4-ylcarbonyl)phenyl]urea MS (m/z): 679.1 (MH⁺)

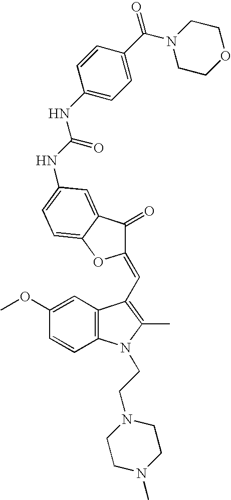

### 1-{(2Z)-2-[(1-Ethyl-5-methoxy-2-methyl-1H-indol-3-yl)methylidene]-3-oxo-2,3-dihydro-1-benzofuran-5-yl}-3-[4-(morpholin-4-ylcarbonyl)phenyl]urea MS (m/z): 581.1 (MH⁺)

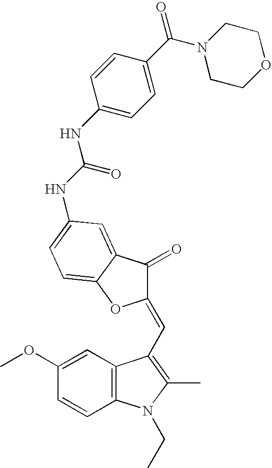

### 1-{(2Z)-2-[(5-Methoxy-1,2-dimethyl-1H-indol-3-yl)methylidene]-3-oxo-2,3-dihydro-1-benzofuran-5-yl}-3-{4-[(4-methylpiperazin-1-yl)carbonyl]phenyl}urea MS (m/z): 580.4 (MH⁺)

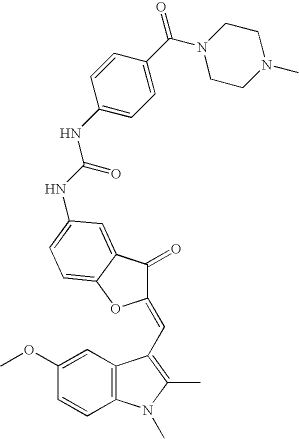

### N-[2-(Dimethylamino)ethyl]-4-[({(2Z)-2-[(1-ethyl-5-methoxy-1H-indol-3-yl)methylene]-3-oxo-2,3-dihydro-1-benzofuran-5-yl}carbamoyl)amino]-N-methyl benzamide MS (m/z): 582.3 (MH⁺)

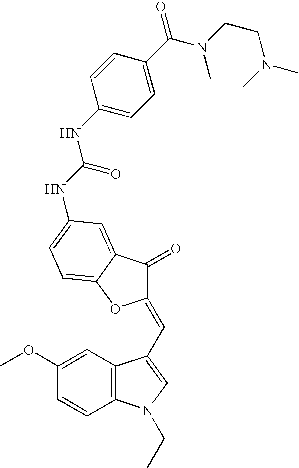

### 1-{(2Z)-2-[(1-Ethyl-5-methoxy-1H-indol-3-yl)methylene]-3-oxo-2,3-dihydro-1-benzofuran-5-yl}-3-{4-[(4-methylpiperazin-1-yl)carbonyl]phenyl}urea MS (m/z): 580.3 (MH⁺)

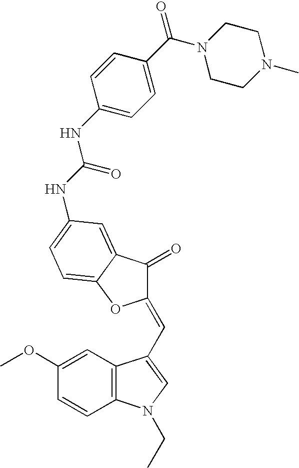

### 1-{(2Z)-2-[(5-Methoxy-1H-indol-3-yl)methylene]-3-oxo-2,3-dihydro-1-benzofuran-5-yl}-3-{4-[(4-methylpiperazin-1-yl)carbonyl]phenyl}urea MS (m/z): 552.3 (MH⁺)

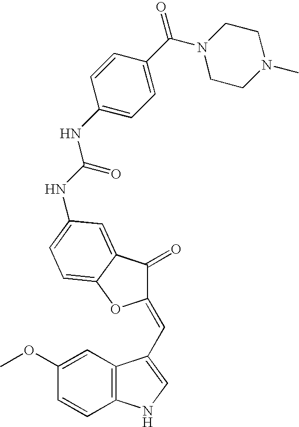

### N-[3-(Dimethylamino)propyl]-4-[({(2Z)-2-[(1-ethyl-5-methoxy-2-methyl-1H-indol-3-yl)methylidene]-3-oxo-2,3-dihydro-1-benzofuran-5-yl}carbamoyl)amino]benzamide MS (m/z): 596.2 (MH⁺)

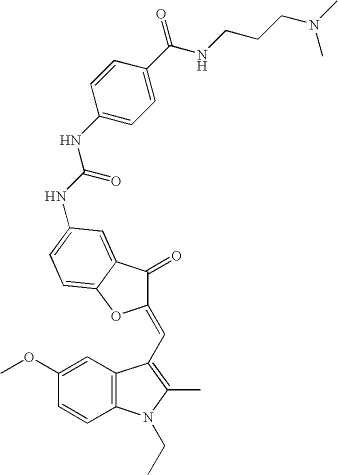

### N-[3-(Dimethylamino)propyl]-4-[({(2Z)-2-[(1-ethyl-5-methoxy-2-methyl-1H-indol-3-yl)methylene]-3-oxo-2,3-dihydro-1-benzofuran-5-yl}carbamoyl)amino]-N-methyl benzamide MS (m/z): 610.3 (MH⁺)

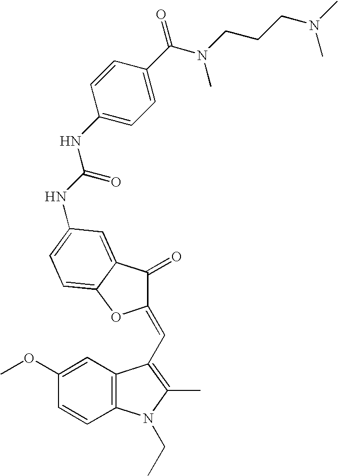

### N-[2-(Dimethylamino)ethyl]-4-[({(2Z)-2-[(1-ethyl-5-methoxy-2-methyl-1H-indol-3-yl)methylidene]-3-oxo-2,3-dihydro-1-benzofuran-5-yl}carbamoyl)amino]-N-methylbenzenesulfonamide: MS (m/z): 632.2 (MH⁺)

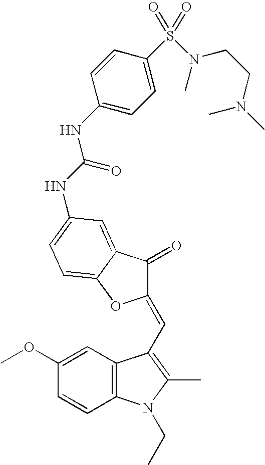

### Synthetic Scheme:

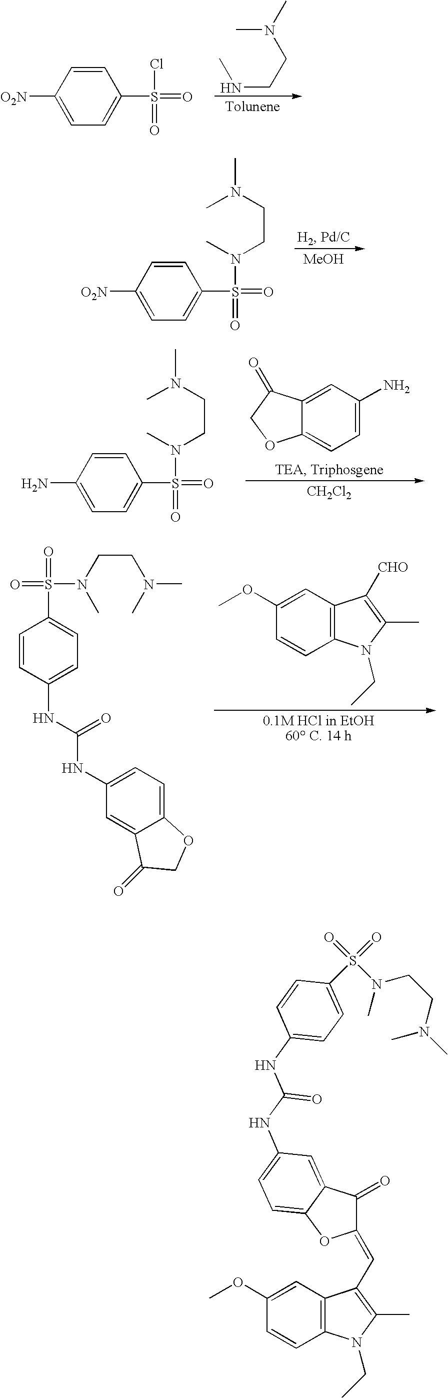

### 1-{(2Z)-2-[(1-Ethyl-5-methoxy-2-methyl-1H-indol-3-yl)methylidene]-3-oxo-2,3-dihydro-1-benzofuran-5-yl}-3-{4-[(4-methylpiperazin-1-yl)sulfonyl]phenyl}urea MS (m/z): 630.3 (MH⁺)

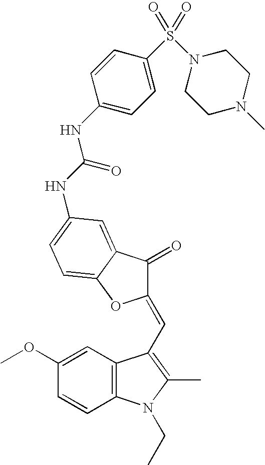

### 4-({[(2Z)-2-({5-Methoxy-2-methyl-1-[2-(4-methylpiperazin-1-yl)ethyl]-1H-indol-3-yl}methylidene)-3-oxo-2,3-dihydro-1-benzofuran-5-yl]carbamoyl}amino)-N-[2-(methylamino)ethyl]benzamide MS (m/z): 666.3 (MH⁺)

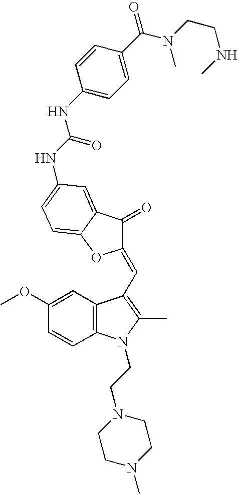

### Synthetic Scheme:

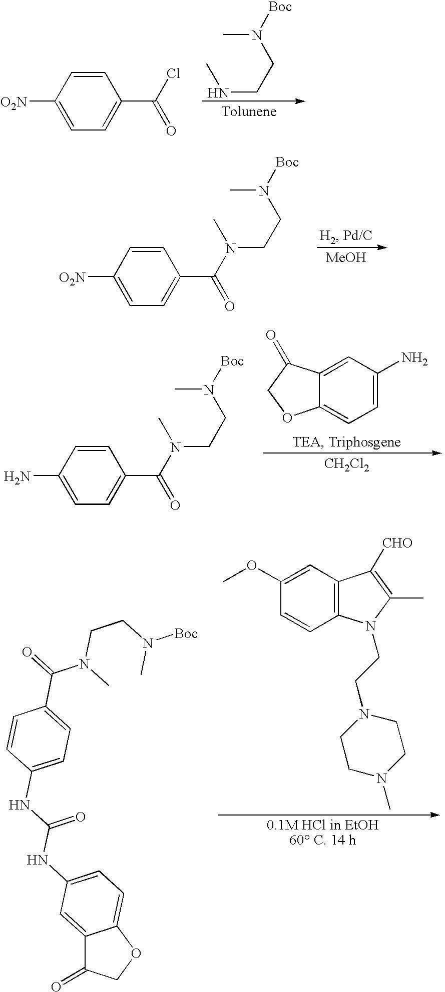

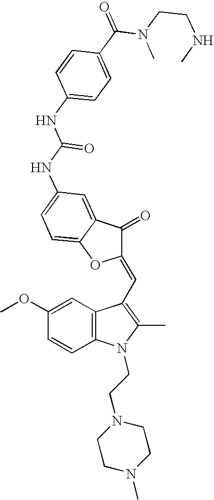

### 4-[({(2Z)-2-[(1-Ethyl-5-methoxy-2-methyl-1H-indol-3-yl)methylene]-3-oxo-2,3-dihydro-1-benzofuran-5-yl}carbamoyl)amino]-N-[2-(methylamino)ethyl]benzamide MS (m/z): 568.3 (MH⁺)

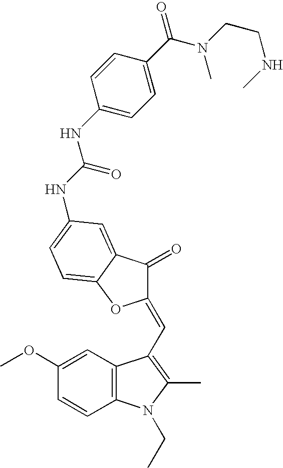

### 4-[({(2Z)-2-[(2-Cyclohexyl-5-methoxy-1H-indol-3-yl)methylene]-3-oxo-2,3-dihydro-1-benzofuran-5-yl}carbamoyl)amino]-N-[2-(methylamino)ethyl]benzamide: MS (m/z): 608.3 (MH⁺)

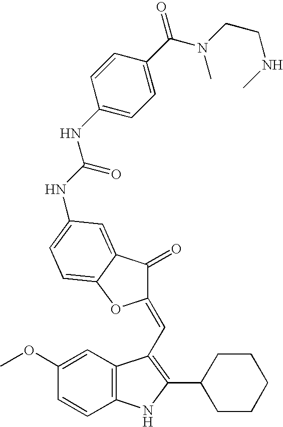

### 4-[({(2Z)-2-[(5-Methoxy-1,2-dimethyl-1H-indol-3-yl)methylene]-3-oxo-2,3-dihydro-1-benzofuran-5-yl}carbamoyl)amino]-N-[2-(methylamino)ethyl]benzamide MS (m/z): 554.3 (MH⁺)

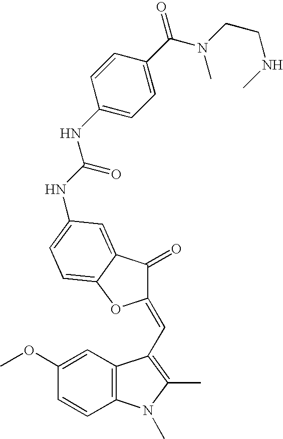

### N-[4-({[(2Z)-2-{[5-Methoxy-2-(1,3,5-trimethyl-1H-pyrazol-4-yl)-1H-indol-3-yl]methylidene}-3-oxo-2,3-dihydro-1-benzofuran-5-yl]carbamoyl}amino)phenyl]-N3,N3-dimethyl-beta-alaninamide MS (m/z): 648.1 (MH⁺)

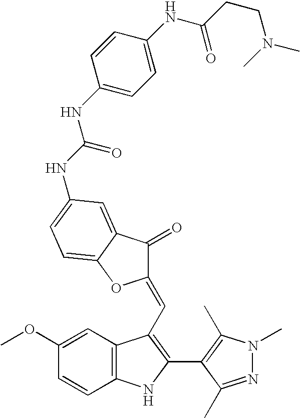

### Synthetic Scheme:

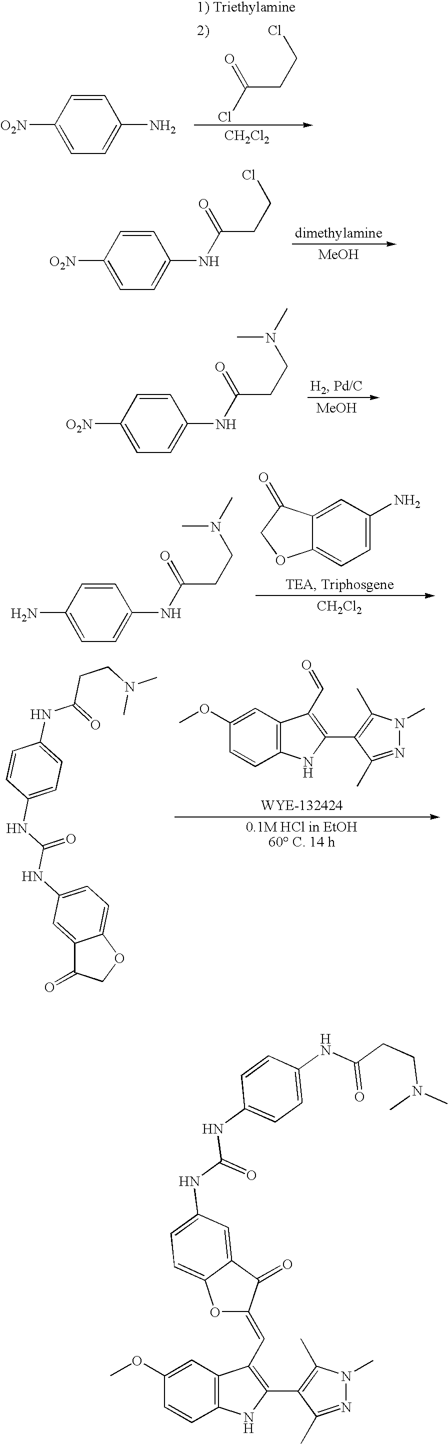

### 3-Chloro-(4-nitrophenyl)propanamide

Into a solution of 4-nitroaniline (1.38 g, 10 mmol) in dichloromethane (50 mL) was added triethylamine (1.01 g, 10 mmol), followed by an addition of chloropropionyl chloride (2.54 g, 20 mmol). The reaction mixture was stirred at room temperature for 4 hours. The resulting reaction mixture was partitioned between dichloromethane and saturated NaHCO₃ aqueous solution. The organic layer was washed with saturated NaCl aqueous solution, dried over MgSO₄, filtered, and concentrated. The residue was stirred with dichloromethane (20 mL) and suction filtered. The solid was dried further in vacuo to give 3-chloro-(4-nitrophenyl)propanamide (1.85 g, 8.09 mmol, 81%) as a yellow solid. Used directly in the next step without further purification.

### 3-(Dimethylamino)-N-(4-nitrophenyl)propanamide

Into a solution of 3-chloro-(4-nitrophenyl)propanamide (228.6 mg, 1.0 mmol) in methanol (20 ml) was added a 2M solution of dimethylamine in THF (5 mL, 10 mmol). The reaction mixture was stirred at room temperature for 14 hours. The resulting reaction mixture was concentrated and partitioned between ethyl acetate and saturated NaHCO₃ aqueous solution. The organic layer was washed with saturated NaCl aqueous solution, dried over MgSO₄, suction filtered, concentrated and dried further in vacuo to give 3-(dimethylamino)-N-(4-nitrophenyl)propanamide (237 mg, 1 mmol, 100%) as a light yellow solid. Used directly in the next step without further purification.

### N-(4-Aminophenyl)-3-(dimethylamino)propanamide

Into a solution of 3-(dimethylamino)-N-(4-nitrophenyl)propanamide (1 g, 4.21 mmol) in anhydrous methanol (40 mL) was added Pd—C (10%, 1 g). A balloon of hydrogen gas (˜2 L) was inserted into the reaction flask. The reaction mixture was stirred under the hydrogen balloon pressure at room temperature for 4 hours. The resulting reaction mixture was suction filtered through a Celite™ bed. The filtrate was concentrated, dried further in vacuo to give N-(4-aminophenyl)-3-(dimethylamino)propanamide (870 mg, 4.2 mmol, 99%) as a light purple solid. Used directly in the next step without further purification.

### 3-(Dimethylamino)-N-{4-[3-(3-oxo-2,3-dihydrobenzofuran-5-yl)ureido]phenyl}propanamide

Into a solution of 5-amino-1-benzofuran-3(2H)-one (149.2 mg, 1.0 mmol) in dichloromethane (30 mL) was added triethylamine (132.5 μL, 1.0 mmol) followed by addition of triphosgene (89 mg, 0.3 mmol). The mixture was stirred at room temperature for 1 hour and N-(4-aminophenyl)-3-(dimethylamino)propanamide (207 mg, 1.0 mmol) was added. The reaction was stirred at room temperature for 2 hours. The resulting reaction mixture was suction filtered. The solid was dried further in vacuo to give 3-(dimethylamino)-N-{4-[3-(3-oxo-2,3-dihydrobenzofuran-5-yl)ureido]phenyl}propanamide (320 mg). Used directly in the next step without further purification.

### N-[4-({[(2Z)-2-({5-Methoxy-2-methyl-1-[2-(4-methylpiperazin-1-yl)ethyl]-1H-indol-3-yl}methylidene)-3-oxo-2,3-dihydro-1-benzofuran-5-yl]carbamoyl}amino)phenyl]-N3, N3-dimethyl-beta-alaninamide MS (m/z): 608.3 (MH⁺)

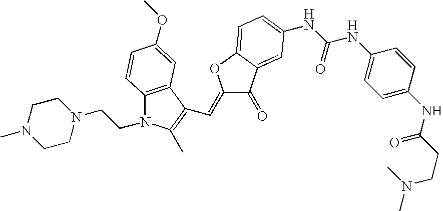

### N-{4-[({(2Z)-2-[(1-Ethyl-5-methoxy-2-methyl-1H-indol-3-yl)methylidene]-3-oxo-2,3-dihydro-1-benzofuran-5-yl}carbamoyl)amino]phenyl}-N3,N3-dimethyl-beta-alaninamide MS (m/z): 582.3 (MH⁺)

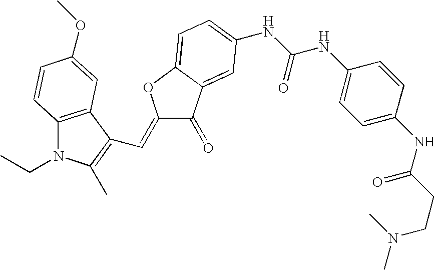

### N-{4-[({(2Z)-2-[(1-Ethyl-5-methoxy-2-methyl-1H-indol-3-yl)methylidene]-3-oxo-2,3-dihydro-1-benzofuran-5-yl}carbamoyl)amino]phenyl}-N,N3,N3-trimethyl-beta-alaninamide MS (m/z): 596.2 (MH⁺)

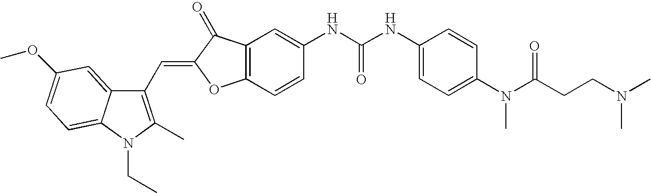

### N-[4-({[(2Z)-2-({5-Methoxy-2-methyl-1-[2-(4-methylpiperazin-1-yl)ethyl]-1H-indol-3-yl}methylidene)-3-oxo-2,3-dihydro-1-benzofuran-5-yl]carbamoyl}amino)phenyl]-N,N3, N3-trimethyl-beta-alaninamide MS (m/z): 347.7 [M+2H]

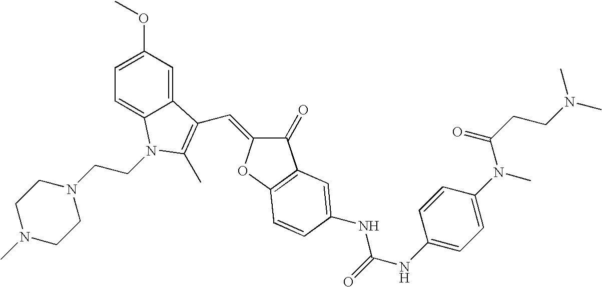

### N-(4-{[(2-{[5-Methoxy-2-(1,3,5-trimethyl-1H-pyrazol-4-yl)-1H-indol-3-yl]methylidene}-3-oxo-2,3-dihydro-1-benzofuran-5-yl)carbamoyl]amino}phenyl)-N,N3,N3-trimethyl-beta-alaninamide MS (m/z): 662.4 (MH⁺)

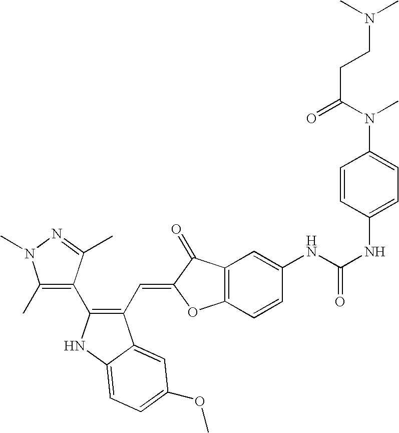

### 1-[(2Z)-2-({5-Methoxy-2-methyl-1-[2-(4-methylpiperazin-1-yl)ethyl]-1H-indol-3-yl}methylene)-6-methyl-3-oxo-2,3-dihydro-1-benzofuran-5-yl]-3-methylurea MS (m/z): 518.3 (MH⁺).

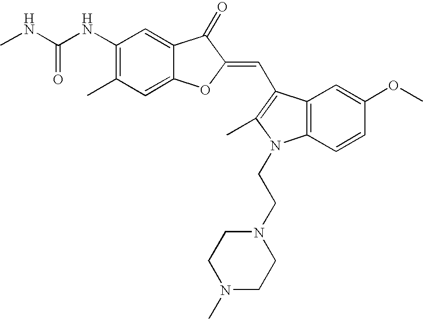

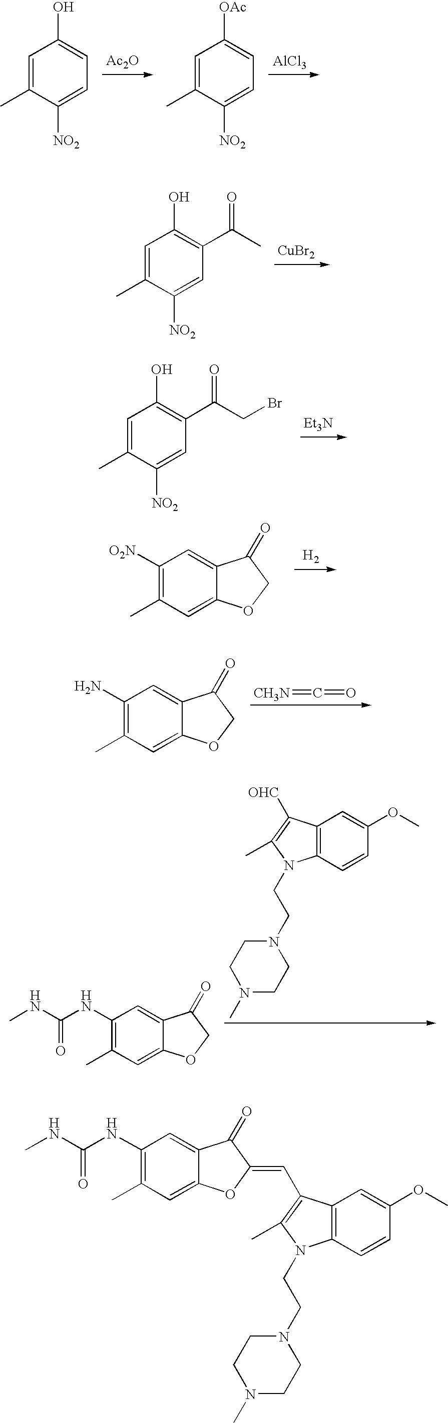

### Step A. 3-Methyl-4-nitrophenyl acetate

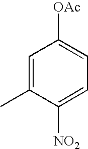

A mixture of 3-methyl-4-nitrophenol (7.7 g, 50 mmol), lithium perchlorate (500 mg), and magnesium sulfate (500 mg) in 50 mL of acetic anhydride was stirred at 80° C. for 30 minutes and concentrated. The residue was partitioned between ethyl acetate and water. The organic layer was dried over magnesium sulfate and filtered through a short pad of silica gel to give 3-methyl-4-nitrophenyl acetate as brown oil. Yield: 94%. MS (m/z): 195.1 (M).

### Step B. 1-(2-Hydroxy-4-methyl-5-nitrophenyl)ethanone

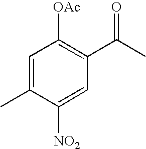

To a mixture of aluminum chloride (1.48 g, 11 mmol) in 12 mL of nitrobenzene was added 3-methyl-4-nitrophenyl acetate (2.15 g, 11 mmol) slowly. The mixture was stirred at 140° C. for 6 hours, and poured into a mixture of 100 g of ice and 60 mL of concentrated HCl. The product was extracted with ethyl acetate and the organic layer was washed with 10% NaOH solution. The alkali solution was neutralized with concentrated HCl, and the product was extracted with ethyl acetate. The organic layer is dried over magnesium sulfate and concentrated. The residue was chromatographed over silica gel, eluting with a gradient of hexanes to 10% ethyl acetate in hexanes to give 1-(2-hydroxy-4-methyl-5-nitrophenyl)ethanone as off-white needles. Yield: 12%. MS (m/z): 194.1 (MH−).

The remaining steps follow the procedure described earlier

### 1-[(2Z)-2-({5-methoxy-2-methyl-1-[2-(4-methylpiperazin-1-yl)ethyl]-1H-indol-3-yl}methylidene)-7-methyl-3-oxo-2,3-dihydro-1-benzofuran-5-yl]-3-methyl urea MS (m/z): 518.3 (MH⁺)

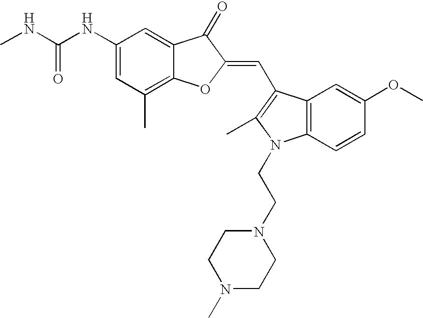

Prepared in the same manner as the previous example, starting from 2-methyl-4-nitrophenol.

### 1-{(2Z)-2-[(1-Ethyl-5-methoxy-2-methyl-1H-indol-3-yl)methylene]-3-oxo-2,3-dihydro-1-benzofuran-5-yl}-3-{4-[(1-methylpiperidin-4-yl)carbonyl]phenyl}urea MS (m/z): 593.3 (M H⁺)

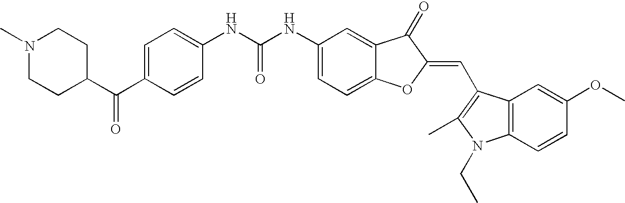

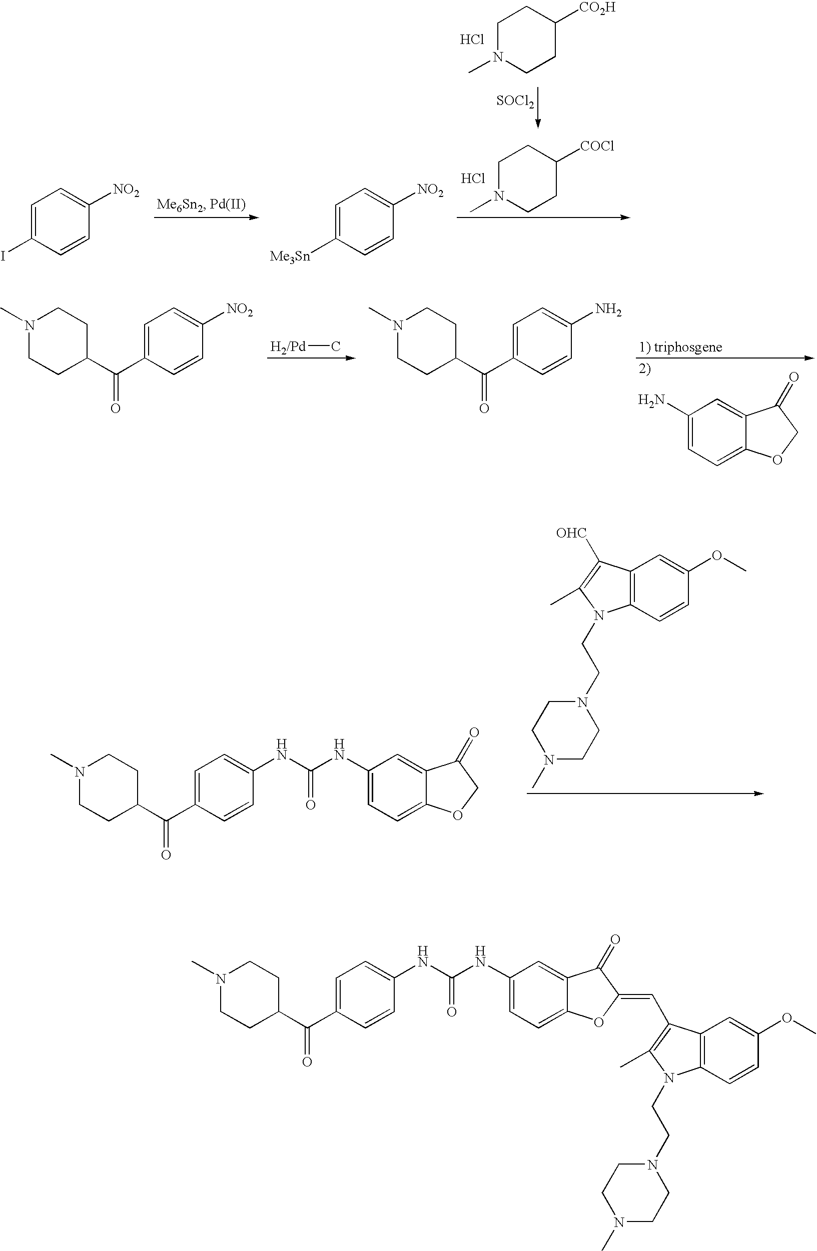

### Step A. (1-Methylpiperidin-4-yl)(4-nitrophenyl)methanone

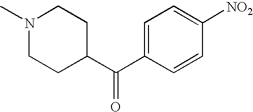

A mixture of 1-methylpiperidine carboxylic acid hydrochloride (1.8 g, 10 mmol) and 20 mL of thionyl chloride was stirred at reflux for 1 hour and concentrated. The crude product was used directly in the next step.

A mixture of 1-iodo-4-nitrobenzene (600 mg, 2.4 mmol), hexamethylditin (1.0 g, 3 mmol), and pi-allyl palladium dichloride dimer (10 mg) in 10 mL of DMF was stirred at room temperature for 2 hours. 1-Methylpiperidine-4-carbonyl chloride hydrochloride (1.0 g, 5 mmol, from previous step) was added and the mixture was stirred at room temperature for 18 hours. The reaction mixture was partitioned between ethyl acetate and water. The organic layer was washed with water (×2) and brine (×2), dried over magnesium sulfate, and concentrated. The residue is chromatographed over silica gel, eluting with a gradient of ethyl acetate to 50% methanol in ethyl acetate to give (1-methylpiperidin-4-yl)(4-nitrophenyl)methanone as a yellow solid. Yield: 41%. MS (m/z): 249.1 (MH⁺).

The remaining steps follow the procedure described earlier.

### N-[2-(Dimethylamino)ethyl]-4-[({(2Z)-2-[(1-ethyl-5-methoxy-2-methyl-1H-indol-3-yl)methylene]-3-oxo-2,3-dihydro-1-benzofuran-5-yl}carbamoyl)amino]-N-methyl benzamide MS (m/z): 596.2 (MH⁺)

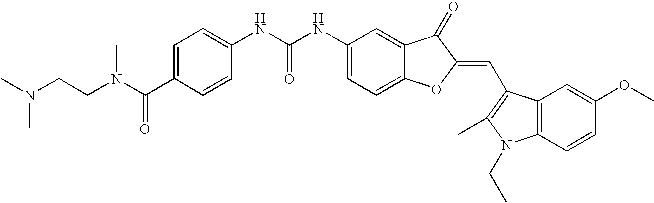

### 1-(4-{[4-(Dimethylamino)piperidin-1-yl]carbonyl}phenyl)-3-{(2Z)-2-[(1-ethyl-5-methoxy-2-methyl-1H-indol-3-yl)methylene]-3-oxo-2,3-dihydro-1-benzofuran-5-yl}urea MS (m/z): 622.3 (MH⁺)

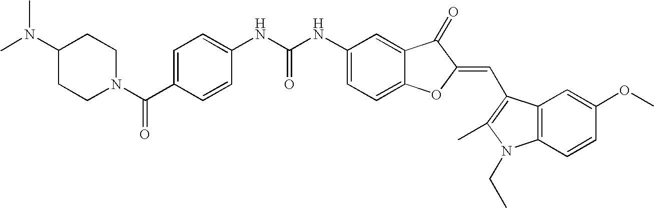

### 1-(4-{[3-(Dimethylamino)propyl](methyl)amino}phenyl)-3-{(2Z)-2-[(1-ethyl-5-methoxy-2-methyl-1H-indol-3-yl)methylene]-3-oxo-2,3-dihydro-1-benzofuran-5-yl}urea MS (m/z): 582.3 (MH⁺)

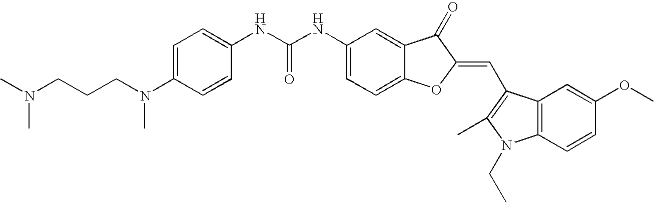

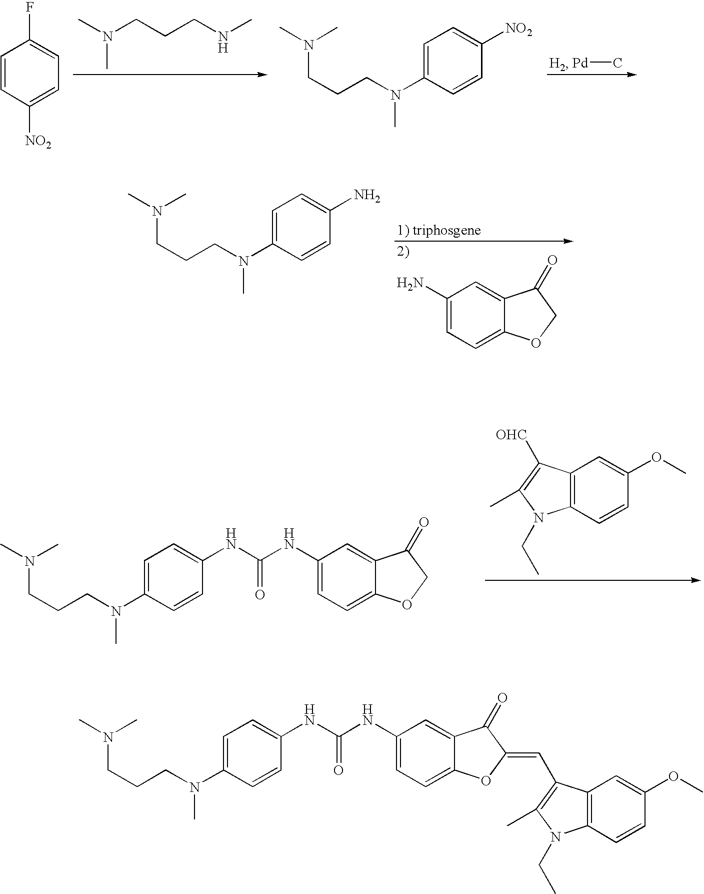

### Step A. N,N,N′-Trimethyl-N′-(4-nitrophenyl)propane-1,3-diamine

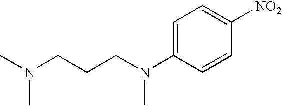

A mixture of 1-fluoro-4-nitrobenzene (705 mg, 5 mmol), N,N,N′-trimethyl-1,3-propanediamine (1 mL, excess) and 1.0 g of potassium carbonate in 50 mL of DMF was stirred at 60° C. for 2 hours and concentrated. The residue was chromatographed over silica gel, eluting with a gradient of ethyl acetate to 50% methanol in ethyl acetate to N,N,N′-trimethyl-N′-(4-nitrophenyl)propane-1,3-diamine as a yellow oil. The product was used directly in the next step.

The remaining steps follow the procedure described earlier.

### 1-{4-[3-(Dimethylamino)propoxy]phenyl}-3-{(2Z)-2-[(1-ethyl-5-methoxy-2-methyl-1H-indol-3-yl)methylene]-3-oxo-2,3-dihydro-1-benzofuran-5-yl}urea MS (m/z): 569.3 (M H⁺)

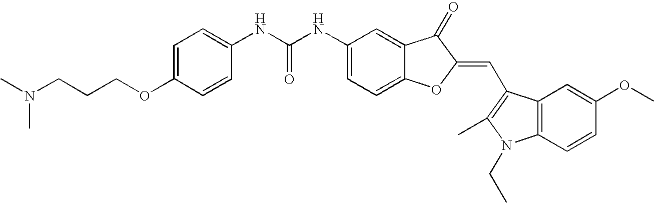

### 1-(4-{[2-(Dimethylamino)ethyl](methyl)amino}phenyl)-3-{(2Z)-2-[(1-ethyl-5-methoxy-2-methyl-1H-indol-3-yl)methylene]-3-oxo-2,3-dihydro-1-benzofuran-5-yl}urea MS (m/z): 568.3 (MH⁺)

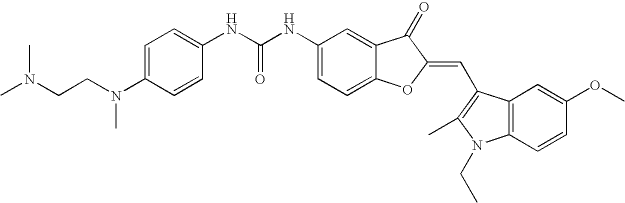

### 1-{4-[2-(dimethylamino)ethoxy]phenyl}-3-{(2Z)-2-[(1-ethyl-5-methoxy-2-methyl-1H-indol-3-yl)methylidene]-3-oxo-2,3-dihydro-1-benzofuran-5-yl}urea MS (m/z): 555.2 (MH⁺)

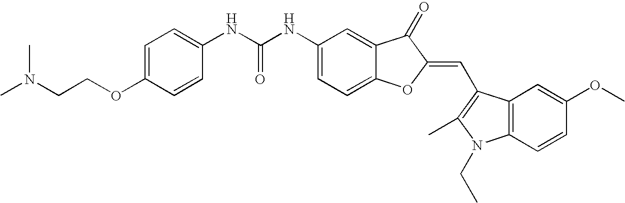

### 1-{4-[4-(Dimethylamino)butoxy]phenyl}-3-{(2Z)-2-[(1-ethyl-5-methoxy-2-methyl-1H-indol-3-yl)methylidene]-3-oxo-2,3-dihydro-1-benzofuran-5-yl}urea MS (m/z): 583.3 (MH⁺)

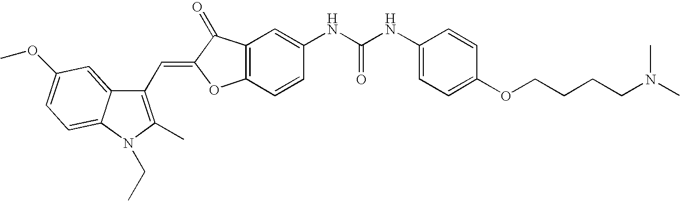

### 1-(4-{[4-(Dimethylamino)butyl](methyl)amino}phenyl)-3-{(2Z)-2-[(1-ethyl-5-methoxy-2-methyl-1H-indol-3-yl)methylidene]-3-oxo-2,3-dihydro-1-benzofuran-5-yl}urea MS (m/z): 596.3 (MH⁺)

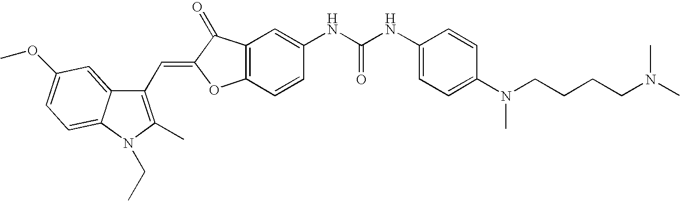

### 1-{4-[4-(Dimethylamino)butoxy]phenyl}-3-[(2Z)-2-({5-methoxy-2-methyl-1-[2-(4-methylpiperazin-1-yl)ethyl]-1H-indol-3-yl}methylidene)-3-oxo-2,3-dihydro-1-benzofuran-5-yl]urea MS (m/z): 341.2 (M2H⁺⁺)

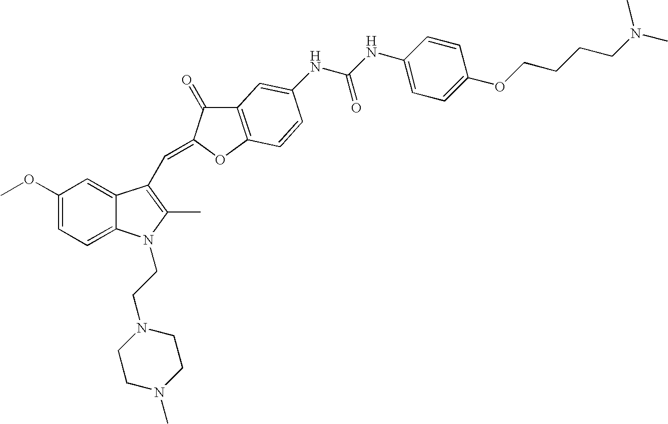

### 1-(4-{[2-(Dimethylamino)ethyl]amino}phenyl)-3-{(2Z)-2-[(1-ethyl-5-methoxy-2-methyl-1H-indol-3-yl)methylidene]-3-oxo-2,3-dihydro-1-benzofuran-5-yl}urea MS (m/z): 554.3 (MH⁺)

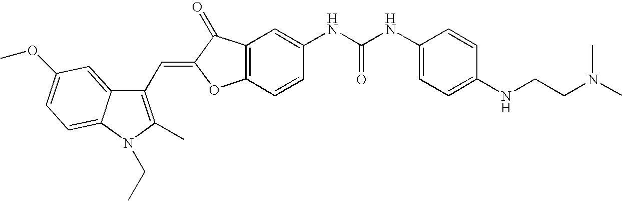

### 1-(4-{[2-(Dimethylamino)ethyl]amino}phenyl)-3-[(2Z)-2-({5-methoxy-2-methyl-1-[2-(4-methylpiperazin-1-yl)ethyl]-1H-indol-3-yl}methylidene)-3-oxo-2,3-dihydro-1-benzofuran-5-yl]urea MS (m/z): 652.4 (MH⁺)

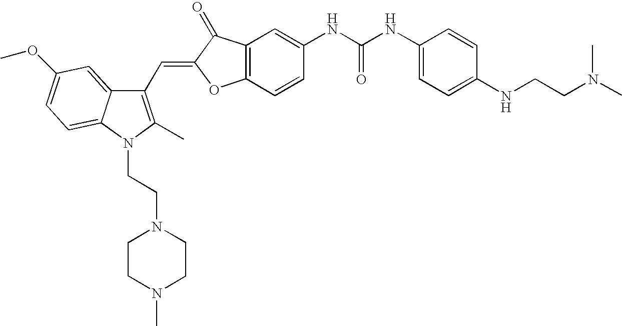

### 1-{(2Z)-2-[(1-Ethyl-5-methoxy-2-methyl-1H-indol-3-yl)methylidene]-3-oxo-2,3-dihydro-1-benzofuran-5-yl}-3-{4-[3-(methylamino)propoxy]phenyl}urea MS (m/z): 555.3 (MH⁺)

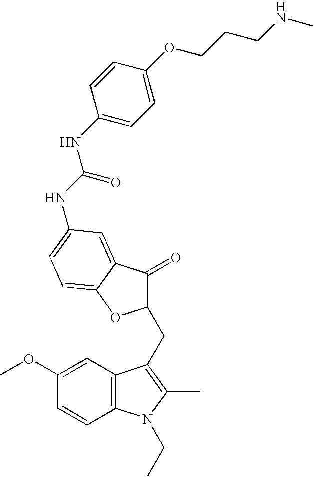

### 1-{4-[4-(Dimethylamino)butyl]phenyl}-3-{(2Z)-2-[(1-ethyl-5-methoxy-2-methyl-1H-indol-3-yl)methylidene]-3-oxo-2,3-dihydro-1-benzofuran-5-yl}urea MS (m/z): 567.3 (MH⁺)

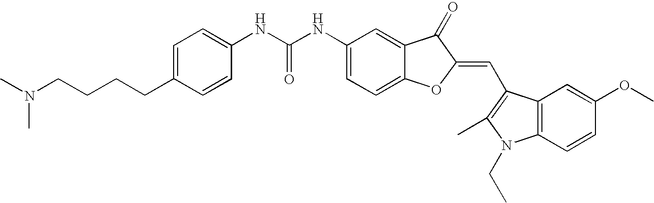

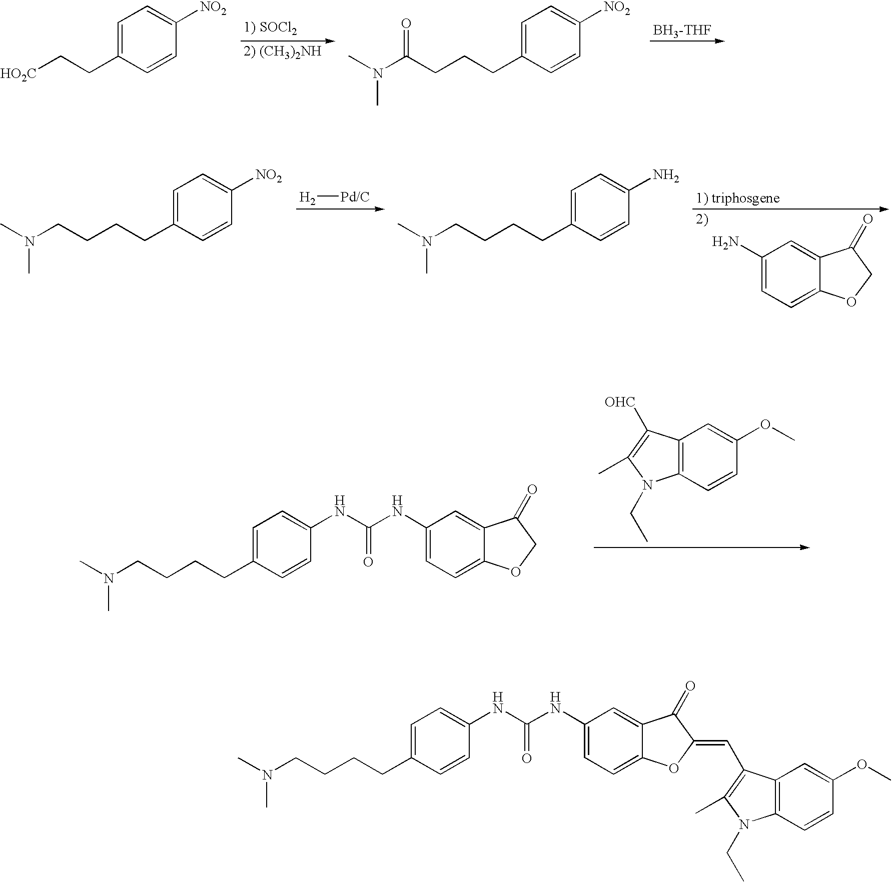

### Step A. N,N-Dimethyl-4-(4-nitrophenyl)butanamide

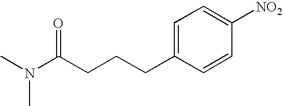

A mixture of 4-(p-nitrophenyl)butyric acid (1.05 g, 5.0 mmol) and 10 mL of thionyl chloride was stirred under reflux for 1 hour and concentrated. The residue was dissolved in 20 mL of THF and dimethyl amine (2 N in THF, 10 mL, 20 mmol) was added. The mixture was stirred at room temperature for 30 minutes, concentrated, and partitioned between ethyl acetate and water. The organic layer was washed with saturated sodium chloride solution, dried over magnesium sulfate, and filtered through a short pad of silica gel to give N,N-dimethyl-4-(4-nitrophenyl)butanamide as a light yellow solid. Yield: 77%.

### Step B. N,N-Dimethyl-4-(4-nitrophenyl)butan-1-amine

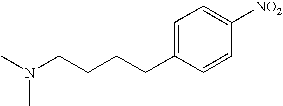

To 25 mL of borane-tetrahydrofuran complex (1.0 M in THF, 25 mmol) at room temperature was added N,N-dimethyl-4-(4-nitrophenyl)butanamide (910 g, 3.85 mmol). The mixture was stirred under reflux for 2 hours, and cooled to 0° C. HCl (2.0 N, 10 mL, 20 mmol) was added, and the mixture was concentrated. To this residue was added conc. HCl (10 mL), and the mixture was reflux for 1 hour and cooled to room temperature. The solution was made alkaline by adding sodium hydroxide, and the product was extracted with ethyl acetate. The organic layer was extracted with 1N HCl, and the aqueous layer was made alkaline by adding sodium hydroxide. The product was extracted with ethyl acetate. The organic layer was washed with 10% NaOH solution. The alkali solution was neutralized with concentrated HCl, and the product was extracted with ethyl acetate. The organic layer was washed with saturated sodium chloride solution, dried over magnesium sulfate, and concentrated to give N,N-dimethyl-4-(4-nitrophenyl)butan-1-amine as a yellow oil. Yield: 56%.

The remaining steps follow the procedure described earlier.

### 1-{4-[3-(Dimethylamino)propyl]phenyl}-3-{(2Z)-2-[(1-ethyl-5-methoxy-2-methyl-1H-indol-3-yl)methylidene]-3-oxo-2,3-dihydro-1-benzofuran-5-yl}urea MS (m/z): 553.2 (MH⁺)

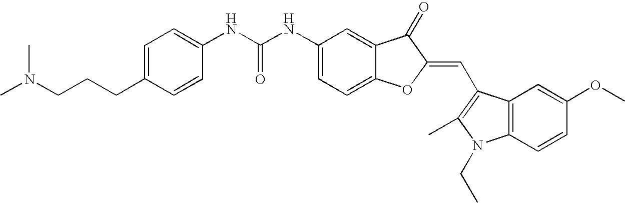

### 1-{4-[2-(Dimethylamino)ethyl]phenyl}-3-{(2Z)-2-[(1-ethyl-5-methoxy-2-methyl-1H-indol-3-yl)methylidene]-3-oxo-2,3-dihydro-1-benzofuran-5-yl}urea MS (m/z): 539.3 (M H⁺)

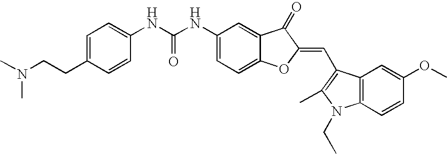

### 1-{4-[(Dimethylamino)methyl]phenyl}-3-{(2Z)-2-[(1-ethyl-5-methoxy-2-methyl-1H-indol-3-yl)methylidene]-3-oxo-2,3-dihydro-1-benzofuran-5-yl}urea MS (m/z): 525.2 (MH⁺)

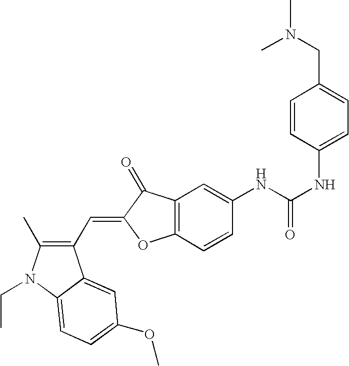

To a stirred solution of triphosgene (31.8 mg, 0.107 mmol) in anhydrous tetrahydrofuran (1 mL) was added 5-aminobenzofuran-3(2H)-one (26.6 mg, 0.179 mmol) at 25° C. The reaction mixture was stirred for 15 minutes and TEA (25 mL, 0.18 mmol, 1 eq) was added and the stirring was continued for an additional 1 hour. Then a mixture of 4-[(dimethylamino)methyl]aniline, HCl (100 mg, 0.536 mmol), TEA (25 mL, 0.18 mmol, 1 eq) in THF (1 mL) was added and stirred for another 2 hours. TEA (406 μL, 2.91 mmol) was added and the mixture was stirred over night. The solvents were removed in a N₂ stream and the crude mixture was purified by semi-prep-HPLC (NH₃-method) to give the desired product as off-white solid. LC/MS didn't show M⁺ only M⁺-NMe2, but ¹H-NMR was consistent.

### 1-[4-(Dimethylamino)phenyl]-3-{(2Z)-2-[(1-ethyl-5-methoxy-2-methyl-1H-indol-3-yl)methylidene]-3-oxo-2,3-dihydro-1-benzofuran-5-yl}urea MS (m/z): 511.2 (MH⁺)

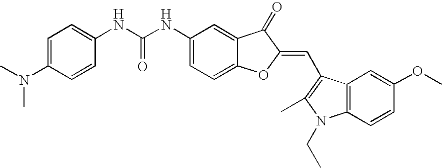

### (Z)-1-(2-((2-(3,5-dimethylisoxazol-4-yl)-5-methoxy-1-(2-(4-methylpiperazin-1-yl)ethyl)-1H-indol-3-yl)methylene)-3-oxo-2,3-dihydrobenzofuran-5-yl)-3-methylurea MS (m/z): 585.3 (MH⁺)

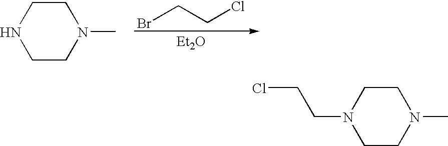

### Preparation of 1-(2-chloroethyl)-4-methylpiperazine

1-Methylpiperazine (22 mL, 200 mmol) added to stirred 1-bromo-2-chloroethane (17 mL, 200 mmol) in Et₂O (200 mL) at 0° C. over 5 minutes. Resulting mixture warmed to room temperature and stirred for 3 days. The mixture filtered and solvent removed from filtrate. Residue from filtrate dissolved in 1:1 THF-hexanes (150 mL) and resulting solution stirred at 45° C. for 2 days. The mixture filtered and filtrate concentrated at 45° C. Yield: 30%. Material used without purification.

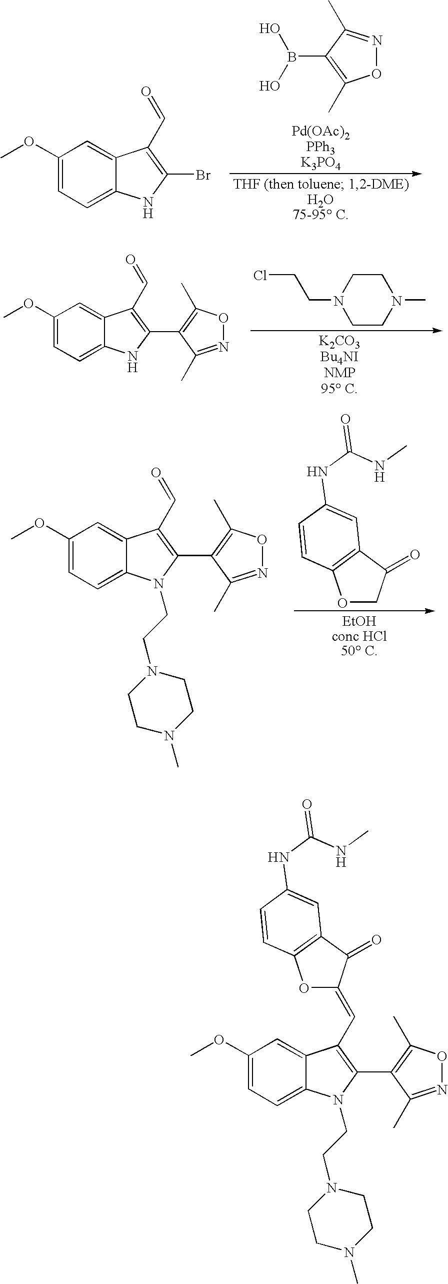

### Preparation of 2-(3,5-dimethylisoxazol-4-yl)-5-methoxy-1H-indole-3-carbaldehyde

A mixture of 2-bromo-5-methoxy-1H-indole-3-carbaldehyde (300 mg, 1.18 mmol), 3,5-dimethylisoxazole-4-boronic acid (333 mg, 2.36 mmol), Pd(OAc)₂ (13 mg, 0.06 mmol), PPh₃ (63 mg, 0.24 mmol), and K₃PO₄ (751 mg, 3.54 mmol) in THF (2.3 mL), and water (2 mL) was stirred under N₂ in a sealed vial at 75° C. overnight. THF was replaced by 1,2-dimethoxyethane (2 mL) and toluene (1 mL) and resulting mixture stirred at 95° C. for 5 hours, then cooled to room temperature. Water (3 mL) added to the mixture and then extracted with EtOAc (3×10 mL). Extracts combined, dried over Na₂SO₄ and concentrated. The mixture purified by silica gel column chromatography (eluent: 45% EtOAc-hexanes). Yield: 79%. MS (m/z): 269.1 (MH−).

### Preparation of 2-(3,5-dimethylisoxazol-4-yl)-5-methoxy-1-(2-(4-methylpiperazin-1-yl)ethyl)-1H-indole-3-carbaldehyde

A mixture of 2-(3,5-dimethylisoxazol-4-yl)-5-methoxy-1H-indole-3-carbaldehyde (102 mg, 0.38 mmol), 1-(2-chloroethyl)-4-methylpiperazine (124 mg, 0.76 mmol), K₂CO₃ (146 mg, 1.06 mmol), and a catalytic amount of Bu₄NI in NMP (0.8 mL) was stirred at 80° C. overnight, then 95° C. over an additional 24 hours. Reaction mixture cooled to room temperature, diluted with EtOAc and extracted using 0.5 M aqueous HCl. Aqueous layer was made basic using saturated aqueous Na₂CO₃ then extracted with EtOAc. Organic layer collected and concentrated. The mixture purified by silica gel column chromatography (eluent: 94:3:3 EtOAc-MeOH-Et₃N). Yield: 27%. MS (m/z): 397.2 (MH⁺).

### (Z)-N-(1-Hydroxy-2-methyl propan-2-yl)-5-methoxy-3-((5-(3-methyl ureido)-3-oxobenzofuran-2(3H)-ylidene)methyl)-1H-indole-2-carboxamide condensation procedure MS (m/z): 479.2 (MH⁺)

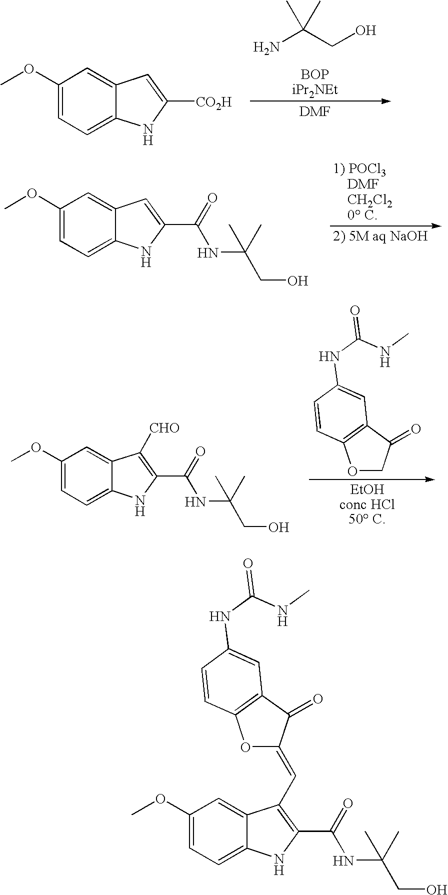

### Preparation of N-(1-hydroxy-2-methylpropan-2-yl)-5-methoxy-1H-indole-2-carboxamide

(Benzotriazol-1-yloxy)tris(dimethylamino)phosphonium hexafluorophosphate (1.97 g, 4.5 mmol) added to a stirred mixture of 5-methoxyindole-2-carboxylic acid (810 mg, 4.2 mmol) and iPr₂NEt (770 μL, 4.7 mmol) in DMF (10 mL) at room temperature. Resulting mixture stirred for 5 minutes then 2-amino-2-methyl-1-propanol (488 μL, 5.1 mmol) added. The mixture stirred overnight then poured into 0.5 M aqueous HCl (25 mL) and extracted with EtOAc (3×50 mL). EtOAc extracts combined, washed with saturated aqueous NaHCO₃ (2×50 mL), water (2×25 mL), and then aqueous NaCl (25 mL). EtOAc extract dried over Na₂SO₄ and concentrated. Resulting tan solid rinsed with EtOAc (2×10 mL) and dried in vacuo. Yield: 65%. MS (m/z): 263.2 (MH⁺).

### Preparation of 3-formyl-N-(1-hydroxy-2-methylpropan-2-yl)-5-methoxy-1H-indole-2-carboxamide

DMF (156 μL, 2.0 mmol) was added to a stirred solution of phosphorus oxychloride (190 μL, 2.0 mmol) in CH₂Cl₂ (0.5 mL) at 0° C. Resulting mixture stirred for 15 minutes then a mixture of N-(1-hydroxy-2-methylpropan-2-yl)-5-methoxy-1H-indole-2-carboxamide (134 mg, 0.51 mmol) in CH₂Cl₂ (2.5 mL) added and the resulting mixture stirred at room temperature for 1 hour. The mixture cooled to 0° C., then 5 M aqueous NaOH added (5 mL) and the mixture stirred for 15 minutes at room temperature. The mixture diluted with water (25 mL) and extracted with CH₂Cl₂ (3×40 mL). CH₂Cl₂ extracts combined, washed with saturated aqueous NaCl (25 mL), dried over Na₂SO₄, and concentrated. Crude mixture purified by preparative HPLC. Yield: 22%. MS (m/z): 291.1 (MH⁺).

### (Z)-1-(2-((2-(3-Cyclopropyl-1,2,4-oxadiazol-5-yl)-5-methoxy-1-(2-(4-methylpiperazin-1-yl)ethyl)-1H-indol-3-yl)methylene)-3-oxo-2,3-dihydrobenzofuran-5-yl)-3-methyl urea MS (m/z): 598.3 (MH⁺)

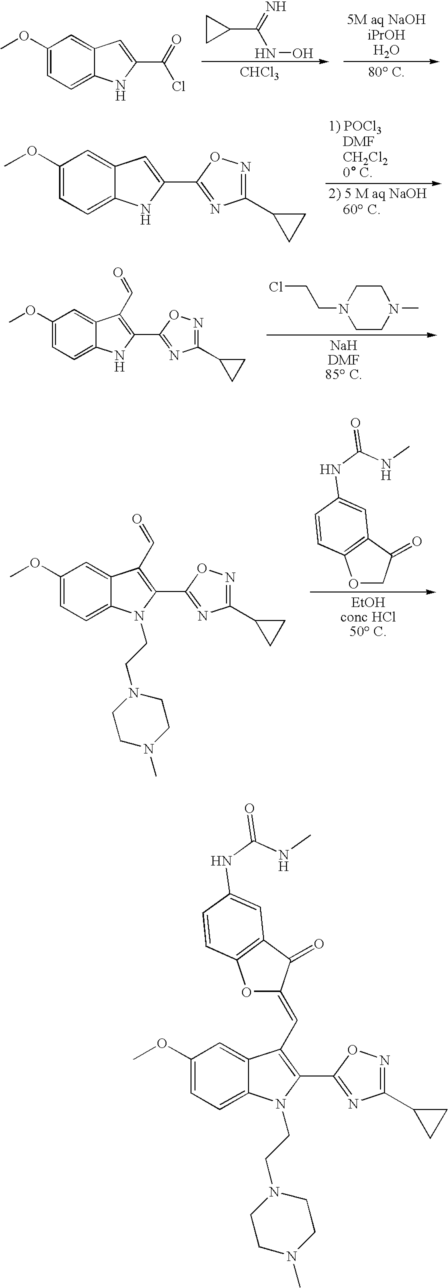

### Preparation of 3-cyclopropyl-5-(5-methoxy-1H-indol-2-yl)-1,2,4-oxadiazole

A mixture of 5-methoxy-1H-indole-2-carbonyl chloride (384 mg, 1.83 mmol) and N′-hydroxycyclopropanecarboximidamide (200 mg, 2.00 mmol) in chloroform (5 mL) was stirred at reflux for 30 minutes then cooled to room temperature and concentrated. The residue treated with isopropyl alcohol (10 mL), water (10 mL), and 5 M aqueous NaOH (5 mL) and the resulting mixture stirred at 80° C. for 45 minutes. Reaction mixture cooled to room temperature, poured into water (50 mL), and extracted with EtOAc (3×50 mL). EtOAc extracts combined, washed with saturated aqueous NaCl (25 mL), dried over Na₂SO₄, and concentrated. The mixture purified by silica gel column chromatography (eluent: 15% EtOAc-hexanes). Yield: 38%. MS (m/z): 256.1 (MH⁺).

### Preparation of 2-(3-cyclopropyl-1,2,4-oxadiazol-5-yl)-5-methoxy-1H-indole-3-carbaldehyde

DMF (241 μL, 3.10 mmol) added to stirred POCl₃ (289 μL, 3.10 mmol) at 0° C. and resulting mixture stirred for 2 minutes then diluted with CH₂Cl₂ (0.5 mL). Resulting mixture stirred for 15 minutes then a solution of 3-cyclopropyl-5-(5-methoxy-1H-indol-2-yl)-1,2,4-oxadiazole (159 mg, 0.62 mmol) in CH₂Cl₂ (2 mL) added and mixture stirred for 1 hour. Reaction mixture treated with water (1 mL), then slowly with 5 M aqueous NaOH (3 mL). Resulting mixture stirred at 60° C. for 5 minutes, cooled to room temperature, diluted with water (25 mL), and extracted with EtOAc (2×50 mL). EtOAc extracts combined, washed with saturated aqueous NaCl (25 mL), dried over Na₂SO₄, and concentrated. Yield: 89%. MS (m/z): 284.1 (MH⁺).

### Preparation of 2-(3-cyclopropyl-1,2,4-oxadiazol-5-yl)-5-methoxy-1-(2-(4-methylpiperazin-1-yl)ethyl)-1H-indole-3-carbaldehyde

A solution of 2-(3-cyclopropyl-1,2,4-oxadiazol-5-yl)-5-methoxy-1H-indole-3-carbaldehyde (32 mg, 0.11 mmol) in DMF (0.5 mL) was added slowly to NaH (excess) and resulting mixture stirred for 5 minutes. 1-(2-Chloroethyl)-4-methylpiperazine (23 mg, 0.14 mmol) was added and the resulting mixture stirred at 85° C. overnight. Additional 1-(2-chloroethyl)-4-methylpiperazine (50 mg, 0.28 mmol) added and mixture stirred for another 24 hours, at 85° C. Reaction mixture cooled to room temperature, diluted with EtOAc and extracted using 0.5 M aqueous HCl. Aqueous layer was made basic using saturated aqueous Na₂CO₃ then extracted with EtOAc. Organic layer collected and concentrated. Yield: 40%. MS (m/z): 410.2 (MH⁺).

### (Z)-1-(2-((2-(3-Isopropyl-1,2,4-oxadiazol-5-yl)-5-methoxy-1-(2-(4-methylpiperazin-1-yl)ethyl)-1H-indol-3-yl)methylene)-3-oxo-2,3-dihydrobenzofuran-5-yl)-3-methyl urea MS (m/z): 600.3 (MH⁺)

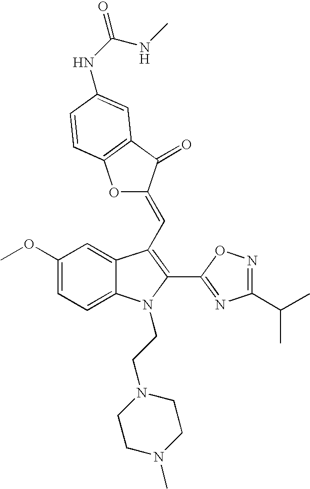

### (Z)-1-(2-((2-tert-Butyl-5-methoxy-1H-indol-3-yl)methylene)-3-oxo-2,3-dihydrobenzofuran-5-yl)-3-methylurea MS (m/z): 420.2 (MH⁺)

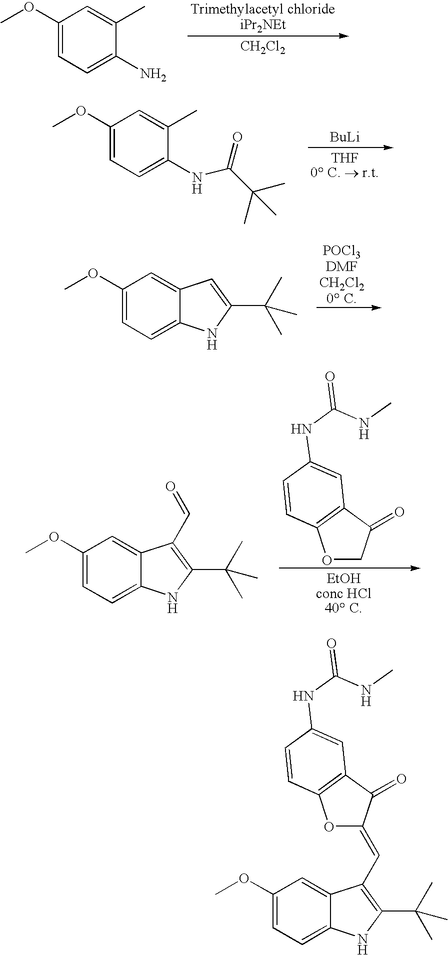

### Preparation of N-(4-methoxy-2-methylphenyl)pivalamide

Trimethylacetyl chloride (2.9 mL, 24 mmol) was added in drops to a stirred solution of 4-methoxy-2-methylaniline (3.1 g, 23 mmol) and iPr₂NEt (4.2 mL, 24 mmol) in CH₂Cl₂ (50 mL) over a period of 2-3 minutes. Resulting mixture stirred for 90 minutes. Solvent removed in vacuo and crude product partitioned between water (25 mL) and 1:1 EtOAc-hexanes (150 mL). Aqueous layer extracted with 1:1 EtOAc-hexanes (50 mL). Organic extracts combined, washed with water (25 mL), saturated aqueous NH₄Cl (25 mL), and saturated aqueous NaCl (25 mL), dried over Na₂SO₄ and concentrated. Yield: >100%. MS (m/z): 222.2 (MH⁺).

### Preparation of 2-tert-butyl-5-methoxy-1H-indole

A solution of BuLi in hexane (2.0 M, 26 mL, 52 mmol) was added slowly to a stirred solution of N-(4-methoxy-2-methylphenyl)pivalamide (˜23 mmol) in THF (100 mL) at 0° C. over a period of 10 minutes. Resulting mixture stirred overnight allowing to warm to room temperature. Reaction mixture slowly poured into stirred 1 M aqueous HCl at 0° C. (150 mL). The mixture extracted with EtOAc (3×100 mL). EtOAc extracts combined, washed with saturated aqueous NaCl, dried over Na₂SO₄, and concentrated. The mixture purified by silica gel column chromatography (eluent: 15% EtOAc-hexanes). Yield: 83%. MS (m/z): 204.2 (MH⁺).

### Preparation of 2-tert-butyl-5-methoxy-1H-indole-3-carbaldehyde

DMF (243 μL, 3.12 mmol) added to stirred POCl₃ (290 μL, 3.12 mmol) at 0° C. and resulting mixture diluted with CH₂Cl₂ (0.5 mL) and stirred for 20 minutes. A solution of 2-tert-butyl-5-methoxy-1H-indole (158 mg, 0.78 mmol) in CH₂Cl₂ (2.5 mL) added and mixture stirred for 45 minutes. Reaction mixture poured into saturated aqueous Na₂CO₃ (25 mL) and extracted with EtOAc (2×50 mL). EtOAc extracts combined, washed with saturated aqueous NaCl (25 mL), dried over Na₂SO₄, and concentrated. Yield: >100%. MS (m/z): 232.2 (MH⁺).

### (Z)-1-(2-((2-Cyano-5-methoxy-1-(2-(4-methylpiperazin-1-yl)ethyl)-1H-indol-3-yl)methylene)-3-oxo-2,3-dihydrobenzofuran-5-yl)-3-methylurea MS (m/z): 515.2 (MH⁺)

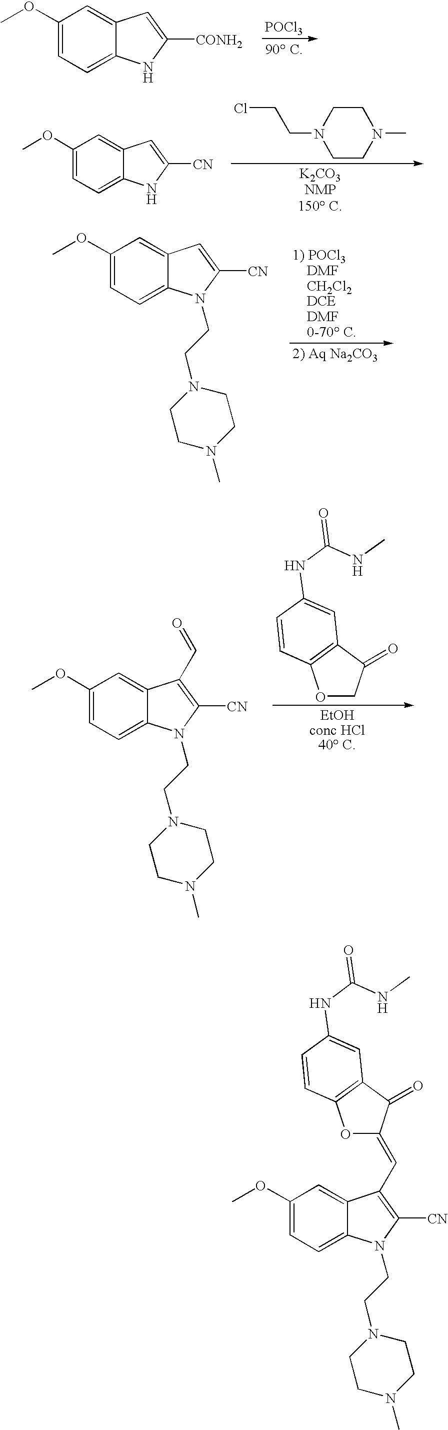

### Preparation of 5-methoxy-1H-indole-2-carbonitrile

Solid 5-methoxy-1H-indole-2-carboxamide (1.59 g, 8.4 mmol) was added to stirred POCl₃ (20 mL) at room temperature. Resulting mixture heated to 90° C., stirred for 45 minutes, and then cooled to room temperature. The mixture poured onto ice (˜100 mL) and let sit for 15 minutes. CH₂Cl₂ (150 mL) added and organic layer washed with 1:1 saturated aqueous Na₂CO₃—H₂O (50 mL), then saturated aqueous NaCl (50 mL), dried over Na₂SO₄, and concentrated. Residue was dried by azeotrope distillation using toluene (2×50 mL) and then dissolved in 60% EtOAc-hexanes and mixture filtered through a plug of SiO₂. Resulting filtrate washed with 1:1 saturated aqueous Na₂CO₃—H₂O until washings remained basic, then washed with saturated aqueous NaCl (50 mL), dried over Na₂SO₄ and concentrated. Yield: 81%. MS (m/z): 171.1 (MH−).

### Preparation of 5-methoxy-1-(2-(4-methylpiperazin-1-yl)ethyl)-1H-indole-2-carbonitrile

A mixture of 5-methoxy-1H-indole-2-carbonitrile (293 mg, 1.70 mmol), K₂CO₃ (1.75 g, 12.7 mmol), and 1-(2-chloroethyl)-4-methylpiperazine (1.41 g, 8.7 mmol) was heated to 150° C. NMP (0.7 mL) was added slowly and the resulting mixture stirred at 150° C. for 2.5 hours. Reaction mixture cooled to room temperature, water added (25 mL) and extracted with EtOAc (2×50 mL). EtOAc extracts combined, washed with water (10 mL), saturated aqueous NaCl (25 mL), dried over Na₂SO₄, and concentrated. The mixture purified by silica gel column chromatography (eluent: 96:2:2 EtOAc-MeOH-Et₃N). Yield: 26%. MS (m/z): 299.2 (MH⁺).

### Preparation of 3-formyl-5-methoxy-1-(2-(4-methylpiperazin-1-yl)ethyl)-1H-indole-2-carbonitrile

DMF (137 μL, 1.76 mmol) added to stirred POCl₃ (164 μL, 1.76 mmol) at 0° C. and resulting mixture diluted with CH₂Cl₂ (0.5 mL) and then stirred for 25 minutes. A solution of 5-methoxy-1-(2-(4-methylpiperazin-1-yl)ethyl)-1H-indole-2-carbonitrile (131 mg, 0.44 mmol) in CH₂Cl₂ (1.5 mL) added and mixture stirred at 50° C. for 3 hours. 1,2-Dichloroethane (1 mL) added and mixture stirred at 70° C. for 2 hours. Additional DMF added (0.8 mL) and mixture stirred at 70° C. for 2.5 days. Additional POCl₃ added (0.5 mL) and mixture stirred at 70° C. for an additional 24 hours. Reaction mixture cooled to room temperature, poured slowly into saturated aqueous Na₂CO₃ (25 mL) and extracted with EtOAc (3×40 mL). EtOAc extracts combined, washed with saturated aqueous NaCl (25 mL), dried over Na₂SO₄, and concentrated. The mixture purified by silica gel column chromatography (eluent: 100% EtOAc to 95:2:3 EtOAc-MeOH-Et₃N gradient). Yield 47%. MS (m/z): 327.2 (MH⁺).

The following benzofuranone analogues were prepared according to the above procedures.

TABLE VII

| | | | HRMS (ESI-FTMS) |
|---|---|---|---|
| Compound | Name | MS (ESI) m/z | [M + H]⁺ |
| 471 | 4-{3-[(4,6-dihydroxy-3-oxo-1-benzofuran-2(3H)- | 419.3 | |
| | ylidene)methyl]-7-ethyl-5-methoxy-1H-indol-1- | | |
| | yl}butanenitrile | | |
| 472 | (2Z)-6-hydroxy-4-methoxy-2-[(5-methoxy-1H-indol- | 338.2 | |
| | 3-yl)methylene]-1-benzofuran-3(2H)-one | | |
| 473 | (2Z)-2-({7-ethyl-5-methoxy-1-[2-(4-methylpiperazin- | 462.4 | |
| | 1-yl)ethyl]-1H-indol-3-yl}methylene)-6-hydroxy-1- | | |
| | benzofuran-3(2H)-one | | |
| 474 | 4-{7-ethyl-3-[(Z)-(6-hydroxy-3-oxo-1-benzofuran- | 403.3 | |
| | 2(3H)-ylidene)methyl]-5-methoxy-1H-indol-1- | | |
| | yl}butanenitrile | | |
| 475 | 4-{7-ethyl-3-[(Z)-(4-hydroxy-3-oxo-1-benzofuran- | 403.3 | |
| | 2(3H)-ylidene)methyl]-5-methoxy-1H-indol-1- | | |
| | yl}butanenitrile | | |
| 476 | (2Z)-2-[(1,4-dimethyl-2-phenyl-1H-indol-3- | 398.3 | |
| | yl)methylene]-4,6-dihydroxy-1-benzofuran-3(2H)- | | |
| | one | | |
| 477 | (2Z)-2-[(1,4-dimethyl-2-pyridin-2-yl-1H-indol-3- | 399.3 | |
| | yl)methylene]-4,6-dihydroxy-1-benzofuran-3(2H)- | | |
| | one | | |
| 478 | (2Z)-4,6-dihydroxy-2-{[1-(2-hydroxyethyl)-4-phenyl- | 414.3 | |
| | 1H-indol-3-yl]methylene}-1-benzofuran-3(2H)-one | | |
| 479 | (2Z)-6-hydroxy-2-[(5-methoxy-7-pyridin-4-yl-1H- | 385.3 | |
| | indol-3-yl)methylene]-1-benzofuran-3(2H)-one | | |
| 480 | (2Z)-6-hydroxy-2-[(5-methoxy-7-pyridin-3-yl-1H- | 385.3 | |
| | indol-3-yl)methylene]-1-benzofuran-3(2H)-one | | |
| 481 | (2Z)-2-[(5-bromo-1H-indol-3-yl)methylene]-7- | 370.1 | |
| | methoxy-1-benzofuran-3(2H)-one | | |
| 482 | 7-hydroxy-2-[(5-methoxy-1H-indol-3-yl)methylene]- | 308.2 | |
| | 1-benzofuran-3(2H)-one | | |
| 483 | 7-methoxy-2-[(5-methoxy-1H-indol-3-yl)methylene]- | 322.2 | |
| | 1-benzofuran-3(2H)-one | | |
| 484 | 1-{(2Z)-2-[(5-methoxy-1H-indol-3-yl)methylene]-3- | 427.1 | |
| | oxo-2,3-dihydro-1-benzofuran-5-yl}-3-pyridin-3- | | |
| | ylurea | | |
| 485 | 1-{(2Z)-2-[(5-methoxy-1H-indol-3-yl)methylene]-3- | 426.1 | |
| | oxo-2,3-dihydro-1-benzofuran-5-yl}-3-phenylurea | | |
| 486 | 1-isopropyl-3-{(2Z)-2-[(5-methoxy-1H-indol-3- | 392.2 | |
| | yl)methylene]-3-oxo-2,3-dihydro-1-benzofuran-5- | | |
| | yl}urea | | |
| 487 | 1-butyl-3-{(2Z)-2-[(5-methoxy-1H-indol-3- | 406.2 | |
| | yl)methylene]-3-oxo-2,3-dihydro-1-benzofuran-5- | | |
| | yl}urea | | |
| 488 | 1-cyclohexyl-3-{(2Z)-2-[(5-methoxy-1H-indol-3- | 432.2 | |
| | yl)methylene]-3-oxo-2,3-dihydro-1-benzofuran-5- | | |
| | yl}urea | | |
| 489 | 1-ethyl-3-{(2Z)-2-[(5-methoxy-1H-indol-3- | 378.1 | |
| | yl)methylene]-3-oxo-2,3-dihydro-1-benzofuran-5- | | |
| | yl}urea | | |
| 490 | methyl ({(2Z)-2-[(5-methoxy-1H-indol-3- | 408.1 | |
| | yl)methylene]-3-oxo-2,3-dihydro-1-benzofuran-5- | | |
| | yl}carbamoyl)carbamate | | |
| 491 | 1-{(2Z)-2-[(5-methoxy-1H-indol-3-yl)methylene]-3- | 456.1 | |
| | oxo-2,3-dihydro-1-benzofuran-5-yl}-3-(4- | | |
| | methoxyphenyl)urea | | |
| 492 | 1-[4-(dimethylamino)phenyl]-3-{(2Z)-2-[(5-methoxy- | 469.2 | |
| | 1H-indol-3-yl)methylene]-3-oxo-2,3-dihydro-1- | | |
| | benzofuran-5-yl}urea | | |
| 493 | 4-{3-[(Z)-(4,6-dihydroxy-3-oxo-1-benzofuran-2(3H)- | 419.3 | |
| | ylidene)methyl]-2-ethyl-5-methoxy-1H-indol-1- | | |
| | yl}butanenitrile | | |
| 494 | 2-[(5-methoxy-1H-indol-3-yl)methylene]-3-oxo-2,3- | 440.1 | |
| | dihydro-1-benzofuran-6-yl | | |
| | trifluoromethanesulfonate | | |
| 495 | 2-[(5-methoxy-1H-indol-3-yl)methylene]-3-oxo-2,3- | 333.1 | |
| | dihydro-1-benzofuran-6-carboxamide | | |
| 496 | (2Z)-4,6-dihydroxy-2-[(1-methyl-4-morpholin-4-yl- | 393.1444 | |
| | 1H-indol-3-yl)methylene]-1-benzofuran-3(2H)-one | | |
| 497 | (2Z)-4,6-dihydroxy-2-[(4-methoxy-1-methyl-2- | 414.3 | |
| | phenyl-1H-indol-3-yl)methylene]-1-benzofuran- | | |
| | 3(2H)-one | | |
| 498 | (2Z)-6-hydroxy-2-[(5-methoxy-7-pyrimidin-5-yl-1H- | 386.3 | |
| | indol-3-yl)methylene]-1-benzofuran-3(2H)-one | | |
| 499 | (2Z)-4,6-dihydroxy-2-[(1-methyl-4-nitro-2-phenyl- | 429.3 | |
| | 1H-indol-3-yl)methylene]-1-benzofuran-3(2H)-one | | |
| 500 | 4-{3-[(Z)-(6-hydroxy-3-oxo-1-benzofuran-2(3H)- | 452.3 | |
| | ylidene)methyl]-5-methoxy-7-pyridin-4-yl-1H-indol- | | |
| | 1-yl}butanenitrile | | |
| 501 | 4-{3-[(Z)-(6-hydroxy-3-oxo-1-benzofuran-2(3H)- | 453.3 | |
| | ylidene)methyl]-5-methoxy-7-pyrimidin-5-yl-1H- | | |
| | indol-1-yl}butanenitrile | | |
| 502 | 4-{3-[(Z)-(6-hydroxy-3-oxo-1-benzofuran-2(3H)- | 452.3 | |
| | ylidene)methyl]-5-methoxy-7-pyridin-3-yl-1H-indol- | | |
| | 1-yl}butanenitrile | | |
| 503 | 4-{3-[(Z)-(4,6-dihydroxy-3-oxo-1-benzofuran-2(3H)- | 469.3 | |
| | ylidene)methyl]-5-methoxy-7-pyrimidin-5-yl-1H- | | |
| | indol-1-yl}butanenitrile | | |
| 504 | 6-hydroxy-2-[(5-methoxy-1H-indol-3-yl)methylene]- | 384.3 | |
| | 4-phenyl-1-benzofuran-3(2H)-one | | |
| 505 | 4-bromo-6-hydroxy-2-[(5-methoxy-1H-indol-3- | 384.0 and 386.0 | |
| | yl)methylene]-1-benzofuran-3(2H)-one | | |
| 506 | 4-ethyl-6-hydroxy-2-[(5-methoxy-1H-indol-3- | 336.3 | |
| | yl)methylene]-1-benzofuran-3(2H)-one | | |
| 507 | (2Z)-6-hydroxy-2-[(5-methoxy-1-methyl-1H-indol-3- | 336.3 | |
| | yl)methylene]-4-methyl-1-benzofuran-3(2H)-one | | |
| 508 | (2Z)-2-[(5-methoxy-1H-indol-3-yl)methylene]-3-oxo- | 315.1 | |
| | 2,3-dihydro-1-benzofuran-6-carbonitrile | | |
| 509 | (2Z)-2-[(5-methoxy-1H-indol-3-yl)methylene]-6-(2H- | 358.1 | |
| | tetrazol-5-yl)-1-benzofuran-3(2H)-one | | |
| 510 | 4-{3-[(Z)-(4,6-dihydroxy-3-oxo-1-benzofuran-2(3H)- | 391.3 | |
| | ylidene)methyl]-5-methoxy-1Hindol-1- | | |
| | yl}butanenitrile | | |
| 511 | 4-{3-[(Z)-(6-hydroxy-3-oxo-1-benzofuran-2(3H)- | 375.3 | |
| | ylidene)methyl]-5-methoxy-1Hindol-1- | | |
| | yl}butanenitrile | | |
| 512 | (2Z)-2-({7-ethyl-5-methoxy-1-[2-(4-methylpiperazin- | 478.5 | |
| | 1-yl)ethyl]-1H-indol-3-yl}methylene)-4,6-dihydroxy- | | |
| | 1-benzofuran-3(2H)-one | | |
| 513 | (2Z)-6-hydroxy-2-[(5-methoxy-1H-indol-3- | 365.3 | |
| | yl)methylene]-N-methyl-3-oxo-2,3-dihydro-1- | | |
| | benzofuran-4-carboxamide | | |
| 514 | 4-{3-[(Z)-(5-hydroxy-3-oxo-1-benzofuran-2(3H)- | 375.3 | |
| | ylidene)methyl]-5-methoxy-1Hindol-1- | | |
| | yl}butanenitrile | | |
| 515 | (2Z)-2-({1-[3-(dimethylamino)propyl]-5-methoxy- | 393.4 | |
| | 1H-indol-3-yl}methylene)-5-hydroxy-1-benzofuran- | | |
| | 3(2H)-one | | |
| 516 | (2Z)-5-bromo-2-({1-[3-(dimethylamino)propyl]-5- | 455.3 | |
| | methoxy-1H-indol-3-yl}methylene)-1-benzofuran- | | |
| | 3(2H)-one | | |
| 517 | (2Z)-6-hydroxy-4-(hydroxymethyl)-2-[(5-methoxy- | 336.2 | |
| | 1H-indol-3-yl)methylene]-1-benzofuran-3(2H)-one | | |
| 518 | (2Z)-6-hydroxy-2-[(5-methoxy-1H-indol-3- | 349.2 | |
| | yl)methylene]-3-oxo-2,3-dihydro-1-benzofuran-4- | | |
| | carboxamide | | |
| 519 | 2Z)-5-hydroxy-2-({5-methoxy-2-methyl-1-[2-(4- | 448.4 | |
| | methylpiperazin-1-yl)ethyl]-1H-indol-3- | | |
| | yl}methylene)-1-benzofuran-3(2H)-one | | |
| 520 | 1-[(2Z)-2-({1-[3-(dimethylamino)propyl]-5-methoxy- | 449.4 | |
| | 1H-indol-3-yl}methylene)-3-oxo-2,3-dihydro-1- | | |
| | benzofuran-5-yl]-3-methylurea | | |
| 521 | (2Z)-2-[(4-amino-1-methyl-2-phenyl-1H-indol-3- | 399.3 | |
| | yl)methylene]-4,6-dihydroxy-1-benzofuran-3(2H)- | | |
| | one | | |
| 522 | (2Z)-5-bromo-2-[(5-methoxy-1H-indol-3- | 370.2 | |
| | yl)methylene]-1-benzofuran-3(2H)-one | | |
| 523 | (2Z)-2-[(5-methoxy-1H-indol-3-yl)methylene]-3-oxo- | 315.2 | |
| | 2,3-dihydro-1-benzofuran-5-carbonitrile | | |
| 524 | (2Z)-2-[(5-methoxy-1H-indol-3-yl)methylene]-5-(1H- | 358.2 | |
| | tetrazol-5-yl)-1-benzofuran-3(2H)-one | | |
| 525 | N-[(2Z)-2-({1-[3-(dimethylamino)propyl]-5-methoxy- | 434.3 | |
| | 1H-indol-3-yl}methylene)-3-oxo-2,3-dihydro-1- | | |
| | benzofuran-5-yl]acetamide | | |
| 526 | 1-{(2Z)-6-hydroxy-2-[(5-methoxy-1H-indol-3- | 380.3 | |
| | yl)methylene]-3-oxo-2,3-dihydro-1-benzofuran-4- | | |
| | yl}-3-methylurea | | |
| 527 | (2Z)-5-bromo-2-[(1-methyl-4-phenyl-1H-indol-3- | 430.0439 | |
| | yl)methylene]-1-benzofuran-3(2H)-one | | |
| 528 | 1-methyl-3-{(2Z)-2-[(1-methyl-4-phenyl-1H-indol-3- | 424.1658 | |
| | yl)methylene]-3-oxo-2,3-dihydro-1-benzofuran-5- | | |
| | yl}urea | | |
| 529 | (2Z)-5,6-dihydroxy-2-[(5-methoxy-1H-indol-3- | 324.3 | |
| | yl)methylene]-1-benzofuran-3(2H)-one | | |
| 530 | (2Z)-5-hydroxy-2-[(1-methyl-4-phenyl-1H-indol-3- | 368.2 | |
| | yl)methylene]-1-benzofuran-3(2H)-one | | |
| 531 | (2Z)-4,6-dihydroxy-2-[(1-methyl-4-phenyl-1H-indol- | 384.1236 | |
| | 3-yl)methylene]-1-benzofuran-3(2H)-one | | |
| 532 | tert-butyl {(2Z)-2-[(1-methyl-4-phenyl-1H-indol-3- | 467.3 | |
| | yl)methylene]-3-oxo-2,3-dihydro-1-benzofuran-5- | | |
| | yl}carbamate | | |
| 533 | 1-[(2Z)-2-({2-cyclohexyl-5-methoxy-1-[2-(4- | 572.4 | |
| | methylpiperazin-1-yl)ethyl]-1H-indol-3- | | |
| | yl}methylene)-3-oxo-2,3-dihydro-1-benzofuran-5- | | |
| | yl]-3-methylurea | | |
| 534 | (2Z)-5-amino-2-[(1-methyl-4-phenyl-1H-indol-3- | 367.2 | |
| | yl)methylene]-1-benzofuran-3(2H)-one | | |
| 535 | 1-{(2Z)-2-[(1-methyl-4-phenyl-1H-indol-3- | 487.2 | |
| | yl)methylene]-3-oxo-2,3-dihydro-1-benzofuran-5- | | |
| | yl}-3-pyridin-3-ylurea | | |
| 536 | 1-{(2Z)-2-[(5-methoxy-1H-indol-3-yl)methylene]-3- | 364.2 | |
| | oxo-2,3-dihydro-1-benzofuran-5-yl}-3-methylurea | | |
| 537 | 1-[(2Z)-2-({1-[3-(dimethylamino)propyl]-5-methoxy- | 435.3 | |
| | 1H-indol-3-yl}methylene)-3-oxo-2,3-dihydro-1- | | |
| | benzofuran-5-yl]urea | | |
| 538 | methyl [(2Z)-2-({1-[3-(dimethylamino)propyl]-5- | 450.3 | |
| | methoxy-1H-indol-3-yl}methylene)-3-oxo-2,3- | | |
| | dihydro-1-benzofuran-5-yl]carbamate | | |
| 539 | (2Z)-2-({1-[3-(dimethylamino)propyl]-5-methoxy- | 434.3 | |
| | 1H-indol-3-yl}methylene)-Nmethyl-3-oxo-2,3- | | |
| | dihydro-1-benzofuran-5-carboxamide | | |
| 540 | 1-[(2Z)-2-({1-[3-(dimethylamino)propyl]-5-methoxy- | 541.4 | |
| | 1H-indol-3-yl}methylene)-3-oxo-2,3-dihydro-1- | | |
| | benzofuran-5-yl]-3-(4-methoxyphenyl)urea | | |
| 541 | (2Z)-5-(hydroxymethyl)-2-({5-methoxy-1-[2-(4- | 448.3 | |
| | methylpiperazin-1-yl)ethyl]-1Hindol-3- | | |
| | yl}methylene)-1-benzofuran-3(2H)-one | | |
| 542 | (2Z)-4,6-dihydroxy-2-({1-methyl-4- | 413.14969 | |
| | [methyl(phenyl)amino]-1H-indol-3-yl}methylene)-1- | | |
| | benzofuran-3(2H)-one | | |
| 543 | 1-ethyl-3-{(2Z)-2-[(1-methyl-4-phenyl-1H-indol-3- | 438.3 | |
| | yl)methylidene]-3-oxo-2,3-dihydro-1-benzofuran-5- | | |
| | yl}urea | | |
| 544 | (2E)-4,6-dihydroxy-2-[(1-methyl-4-phenyl-1H-indol- | 384.2 | |
| | 3-yl)methylene]-1-benzofuran-3(2H)-one-(2Z)-4,6- | | |
| | dihydroxy-2-[(1-methyl-4-phenyl-1H-indol-3- | | |
| | yl)methylene]-1-benzofuran-3(2H)-one (1:1) | | |
| 545 | (2Z)-6-hydroxy-2-({5-methoxy-7-[(1E)-3-(4- | 446.20626 | |
| | methylpiperazin-1-yl)prop-1-en-1-yl]-1H-indol-3- | | |
| | yl}methylene)-1-benzofuran-3(2H)-one | | |
| 546 | (2Z)-2-({5-methoxy-1-[2-(4-methylpiperazin-1- | 491.2 | |
| | yl)ethyl]-1H-indol-3-yl}methylene)-3-oxo-2,3- | | |
| | dihydro-1-benzofuran-5-yl methylcarbamate | | |
| 547 | 1-[(2Z)-2-({1-[3-(dimethylamino)propyl]-5-methoxy- | 463.3 | |
| | 1H-indol-3-yl}methylene)-3-oxo-2,3-dihydro-1- | | |
| | benzofuran-5-yl]-3-ethylurea | | |
| 548 | 1-[(2Z)-2-({5-methoxy-1-[2-(4-methylpiperazin-1- | 514.3 | |
| | yl)ethyl]-1H-indol-3-yl}methylene)-3-oxo-2,3- | | |
| | dihydro-1-benzofuran-5-yl]-3-prop-2-yn-1-ylurea | | |
| 549 | 1-(2-aminoethyl)-3-[(2Z)-2-({5-methoxy-1-[2-(4- | 519.4 | |
| | methylpiperazin-1-yl)ethyl]-1Hindol-3- | | |
| | yl}methylene)-3-oxo-2,3-dihydro-1-benzofuran-5- | | |
| | yl]urea | | |
| 550 | 1-allyl-3-[(2Z)-2-({5-methoxy-1-[2-(4- | 516.4 | |
| | methylpiperazin-1-yl)ethyl]-1H-indol-3- | | |
| | yl}methylene)-3-oxo-2,3-dihydro-1-benzofuran-5- | | |
| | yl]urea | | |
| 551 | 1-[(2Z)-2-({5-methoxy-1-[2-(4-methylpiperazin-1- | 476.3 | |
| | yl)ethyl]-1H-indol-3-yl}methylene)-3-oxo-2,3- | | |
| | dihydro-1-benzofuran-5-yl]urea | | |
| 552 | 1-azetidin-3-yl-3-[(2Z)-2-({5-methoxy-1-[2-(4- | 531.4 | |
| | methylpiperazin-1-yl)ethyl]-1Hindol-3- | | |
| | yl}methylene)-3-oxo-2,3-dihydro-1-benzofuran-5- | | |
| | yl]urea | | |
| 553 | 1-[(2Z)-2-({5-methoxy-1-[2-(4-methylpiperazin-1- | 490.3 | |
| | yl)ethyl]-1H-indol-3-yl}methylene)-3-oxo-2,3- | | |
| | dihydro-1-benzofuran-5-yl]-3-methylurea | | |
| 554 | 1-[(2Z)-2-({1-[3-(dimethylamino)propyl]-5-methoxy- | 479.2 | |
| | 1H-indol-3-yl}methylene)-3-oxo-2,3-dihydro-1- | | |
| | benzofuran-5-yl]-3-(2-hydroxyethyl)urea | | |
| 555 | (2Z)-6-hydroxy-2-({5-methoxy-7-[(1E)-3-piperidin-1- | 431.19514 | |
| | ylprop-1-en-1-yl]-1H-indol-3-yl}methylene)-1- | | |
| | benzofuran-3(2H)-one | | |
| 556 | 1-[(2Z)-2-({5-methoxy-2-methyl-1-[2-(4- | 504.26025 | |
| | methylpiperazin-1-yl)ethyl]-1H-indol-3- | | |
| | yl}methylene)-3-oxo-2,3-dihydro-1-benzofuran-5- | | |
| | yl]-3-methylurea | | |
| 557 | (2E)-4,6-dihydroxy-2-[(1-methyl-4-phenyl-1H-indol- | 384.12296 | |
| | 3-yl)methylene]-1-benzofuran-3(2H)-one | | |
| 558 | (2Z)-2-({5-methoxy-2-methyl-1-[2-(4- | 581.4 | |
| | methylpiperazin-1-yl)ethyl]-1H-indol-3- | | |
| | yl}methylene)-3-oxo-2,3-dihydro-1-benzofuran-6-yl | | |
| | methyl(phenyl)carbamate | | |
| 559 | 1-[(2Z)-2-({2-cyclopropyl-5-methoxy-1-[2-(4- | 530.1 | |
| | methylpiperazin-1-yl)ethyl]-1Hindol-3- | | |
| | yl}methylene)-3-oxo-2,3-dihydro-1-benzofuran-5- | | |
| | yl]-3-methylurea | | |
| 560 | 1-cyclopropyl-3-[(2Z)-2-({1-[3- | 475.3 | |
| | (dimethylamino)propyl]-5-methoxy-1H-indol-3- | | |
| | yl}methylene)-3-oxo-2,3-dihydro-1-benzofuran-5- | | |
| | yl]urea | | |
| 561 | 1-[(2Z)-2-({2-cyclopentyl-5-methoxy-1-[2-(4- | 558.3 | |
| | methylpiperazin-1-yl)ethyl]-1Hindol-3- | | |
| | yl}methylene)-3-oxo-2,3-dihydro-1-benzofuran-5- | | |
| | yl]-3-methylurea | | |
| 562 | 1-[(2Z)-2-({2-cyclobutyl-5-methoxy-1-[2-(4- | 544.3 | |
| | methylpiperazin-1-yl)ethyl]-1H-indol-3- | | |
| | yl}methylene)-3-oxo-2,3-dihydro-1-benzofuran-5- | | |
| | yl]-3-methylurea | | |
| 563 | 1-[(2Z)-2-({5-methoxy-1-[2-(4-methylpiperazin-1- | 533.2 | |
| | yl)ethyl]-1H-indol-3-yl}methylene)-3-oxo-2,3- | | |
| | dihydro-1-benzofuran-5-yl]-3-[2- | | |
| | (methylamino)ethyl]urea | | |
| 564 | 1-[(2Z)-2-({5-methoxy-1-[2-(4-methylpiperazin-1- | 547.4 | |
| | yl)ethyl]-1H-indol-3-yl}methylene)-3-oxo-2,3- | | |
| | dihydro-1-benzofuran-5-yl]-3-[3- | | |
| | (methylamino)propyl]urea | | |
| 565 | 1-(4-aminobutyl)-3-[(2Z)-2-({5-methoxy-1-[2-(4- | 547.4 | |
| | methylpiperazin-1-yl)ethyl]-1Hindol-3- | | |
| | yl}methylene)-3-oxo-2,3-dihydro-1-benzofuran-5- | | |
| | yl]urea | | |
| 566 | 1-(3-aminopropyl)-3-[(2Z)-2-({5-methoxy-1-[2-(4- | 267.1 | |
| | methylpiperazin-1-yl)ethyl]-1Hindol-3- | | |
| | yl}methylene)-3-oxo-2,3-dihydro-1-benzofuran-5- | | |
| | yl]urea | | |
| 567 | 1-[(2Z)-2-({5-methoxy-7-[(1E)-3-morpholin-4- | 489.21135 | |
| | ylprop-1-en-1-yl]-1H-indol-3-yl}methylene)-3-oxo- | | |
| | 2,3-dihydro-1-benzofuran-5-yl]-3-methylurea | | |
| 568 | 1-[(2Z)-2-({1-[3-(dimethylamino)propyl]-5-methoxy- | 493.4 | |
| | 1H-indol-3-yl}methylene)-3-oxo-2,3-dihydro-1- | | |
| | benzofuran-5-yl]-3-(3-hydroxypropyl)urea | | |
| 569 | 1-[3-(dimethylamino)propyl]-3-[(2Z)-2-({5-methoxy- | 281.2 | |
| | 1-[2-(4-methylpiperazin-1-yl)ethyl]-1H-indol-3- | | |
| | yl}methylene)-3-oxo-2,3-dihydro-1-benzofuran-5- | | |
| | yl]urea | | |
| 570 | 1-[(2Z)-2-{[5-methoxy-7-(morpholin-4-ylmethyl)-1H- | 463.19873 | |
| | indol-3-yl]methylene}-3-oxo-2,3-dihydro-1- | | |
| | benzofuran-5-yl]-3-methylurea | | |
| 571 | 1-[(2Z)-2-({5-methoxy-7-[(4-methylpiperazin-1- | 476.23028 | |
| | yl)methyl]-1H-indol-3-yl}methylene)-3-oxo-2,3- | | |
| | dihydro-1-benzofuran-5-yl]-3-methylurea | | |
| 572 | 1-[(2Z)-2-({5-methoxy-1-methyl-7-[3-(4- | 518.27616 | |
| | methylpiperazin-1-yl)propyl]-1H-indol-3- | | |
| | yl}methylene)-3-oxo-2,3-dihydro-1-benzofuran-5- | | |
| | yl]-3-methylurea | | |
| 573 | 1-[(2Z)-2-({7-[(1E)-3-(dimethylamino)prop-1-en-1- | 447.20313 | |
| | yl]-5-methoxy-1H-indol-3-yl}methylene)-3-oxo-2,3- | | |
| | dihydro-1-benzofuran-5-yl]-3-methylurea | | |
| 574 | 1-[2-(dimethylamino)ethyl]-3-[(2Z)-2-({5-methoxy-1- | 274.2 | |
| | [2-(4-methylpiperazin-1-yl)ethyl]-1H-indol-3- | | |
| | yl}methylene)-3-oxo-2,3-dihydro-1-benzofuran-5- | | |
| | yl]urea | | |
| 575 | 1-[(2Z)-2-({5-methoxy-1-[2-(4-methylpiperazin-1- | 566.1 | |
| | yl)ethyl]-2-phenyl-1H-indol-3-yl}methylene)-3-oxo- | | |
| | 2,3-dihydro-1-benzofuran-5-yl]-3-methylurea | | |
| 576 | 1-(2-aminoethyl)-3-[(2Z)-2-({5-methoxy-1-[2-(4- | 298.2 | |
| | methylpiperazin-1-yl)ethyl]-2-phenyl-1H-indol-3- | | |
| | yl}methylene)-3-oxo-2,3-dihydro-1-benzofuran-5- | | |
| | yl]urea | | |
| 577 | 1-(2-aminoethyl)-3-[(2Z)-2-({2-cyclopentyl-5- | 294.2 | |
| | methoxy-1-[2-(4-methylpiperazin-1-yl)ethyl]-1H- | | |
| | indol-3-yl}methylene)-3-oxo-2,3-dihydro-1- | | |
| | benzofuran-5-yl]urea | | |
| 578 | 1-[(2Z)-2-{[5-methoxy-1-(3-piperidin-1-ylpropyl)-1H- | 489.1 | |
| | indol-3-yl]methylene}-3-oxo-2,3-dihydro-1- | | |
| | benzofuran-5-yl]-3-methylurea | | |
| 579 | 1-[(2Z)-2-{[1-(3-cyanopropyl)-5-methoxy-1H-indol- | (FTMS), | |
| | 3-yl]methylene}-3-oxo-2,3-dihydro-1-benzofuran-5- | 431.17134 | |
| | yl]-3-methylurea | | |
| 580 | 1-[(2Z)-2-{[5-methoxy-1-(3-morpholin-4-ylpropyl)- | 491.1 | |
| | 1H-indol-3-yl]methylene}-3-oxo-2,3-dihydro-1- | | |
| | benzofuran-5-yl]-3-methylurea | | |
| 581 | 1-[(2Z)-2-{[2-(3,5-dimethylisoxazol-4-yl)-5-methoxy- | (FTMS) | |
| | 1H-indol-3-yl]methylene}-3-oxo-2,3-dihydro-1- | 459.16704 | |
| | benzofuran-5-yl]-3-methylurea | | |
| 582 | (2Z)-6-hydroxy-2-({5-methoxy-1-methyl-7-[(1E)-3- | (FTMS) | |
| | (4-methylpiperazin-1-yl)prop-1-en-1-yl]-1H-indol-3- | 460.22309 | |
| | yl}methylene)-1-benzofuran-3(2H)-one | | |
| 583 | 1-[(2Z)-2-({5-methoxy-7-[3-(4-methylpiperazin-1- | (FTMS) | |
| | yl)propyl]-1H-indol-3-yl}methylene)-3-oxo-2,3- | 504.26092 | |
| | dihydro-1-benzofuran-5-yl]-3-methylurea | | |
| 584 | 1-[(2Z)-2-({2-cyclohexyl-5-methoxy-1-[2-(4- | 615.4 | |
| | methylpiperazin-1-yl)ethyl]-1H-indol-3- | | |
| | yl}methylene)-3-oxo-2,3-dihydro-1-benzofuran-5- | | |
| | yl]-3-[2-(methylamino)ethyl]urea | | |
| 585 | 1-[(2Z)-2-{[5-methoxy-2-methyl-1-(3-morpholin-4- | 505.3 | |
| | ylpropyl)-1H-indol-3-yl]methylene}-3-oxo-2,3- | | |
| | dihydro-1-benzofuran-5-yl]-3-methylurea | | |
| 586 | 1-[(2Z)-2-({5-methoxy-2-methyl-1-[3-(4- | 518.3 | |
| | methylpiperazin-1-yl)propyl]-1H-indol-3- | | |
| | yl}methylene)-3-oxo-2,3-dihydro-1-benzofuran-5- | | |
| | yl]-3-methylurea | | |
| 587 | 1-[(2Z)-2-({1-[3-(4-hydroxypiperidin-1-yl)propyl]-5- | 519.3 | |
| | methoxy-2-methyl-1H-indol-3-yl}methylene)-3-oxo- | | |
| | 2,3-dihydro-1-benzofuran-5-yl]-3-methylurea | | |
| 588 | 1-[(2Z)-2-{[5-methoxy-2-methyl-1-(3-piperidin-1- | 503.3 | |
| | ylpropyl)-1H-indol-3-yl]methylene}-3-oxo-2,3- | | |
| | dihydro-1-benzofuran-5-yl]-3-methylurea | | |
| 589 | 1-[(2Z)-2-({1-[3-(3-hydroxypyrrolidin-1-yl)propyl]-5- | 505.3 | |
| | methoxy-2-methyl-1H-indol-3-yl}methylene)-3-oxo- | | |
| | 2,3-dihydro-1-benzofuran-5-yl]-3-methylurea | | |
| 590 | 1-[(2Z)-2-({5-methoxy-1-[2-(4-methylpiperazin-1- | 567.2 | |
| | yl)ethyl]-2-pyridin-4-yl-1Hindol-3-yl}methylene)-3- | | |
| | oxo-2,3-dihydro-1-benzofuran-5-yl]-3-methylurea | | |
| 591 | 1-[(2Z)-2-{[2-(3-isopropyl-1,2,4-oxadiazol-5-yl)-5- | (FTMS) | |
| | methoxy-1H-indol-3-yl]methylene}-3-oxo-2,3- | 474.17679 | |
| | dihydro-1-benzofuran-5-yl]-3-methylurea | | |
| 592 | 1-[(2Z)-2-{[2-(3-cyclopropyl-1,2,4-oxadiazol-5-yl)-5- | (FTMS) | |
| | methoxy-1H-indol-3-yl]methylene}-3-oxo-2,3- | 472.1612 | |
| | dihydro-1-benzofuran-5-yl]-3-methylurea | | |
| 593 | 1-[(2Z)-2-{[5-methoxy-2-(3-propyl-1,2,4-oxadiazol- | (FTMS) | |
| | 5-yl)-1H-indol-3-yl]methylene}-3-oxo-2,3-dihydro-1- | 474.17765 | |
| | benzofuran-5-yl]-3-methylurea | | |
| 594 | 1-[(2Z)-2-({5-methoxy-7-[(1E)-3-(4-methylpiperazin- | (FTMS) | |
| | 1-yl)prop-1-en-1-yl]-1Hindol-3-yl}methylene)-3-oxo- | 502.24484 | |
| | 2,3-dihydro-1-benzofuran-5-yl]-3-methylurea | | |
| 595 | 1-{(2Z)-2-[(7-cyano-5-methoxy-1H-indol-3- | (FTMS) | |
| | yl)methylene]-3-oxo-2,3-dihydro-1-benzofuran-5- | 389.12413 | |
| | yl}-3-methylurea | | |
| 596 | 1-[(2Z)-2-({5-methoxy-1-[2-(4-methylpiperazin-1- | 567.3 | |
| | yl)ethyl]-2-pyridin-3-yl-1Hindol-3-yl}methylene)-3- | | |
| | oxo-2,3-dihydro-1-benzofuran-5-yl]-3-methylurea | | |
| 597 | 1-[(2Z)-2-{[5-methoxy-2-methyl-1-(3-pyrrolidin-1- | 489.3 | |
| | ylpropyl)-1H-indol-3-yl]methylidene}-3-oxo-2,3- | | |
| | dihydro-1-benzofuran-5-yl]-3-methylurea | | |
| 598 | 1-[(2Z)-2-({1-[3-(1H-imidazol-1-yl)propyl]-5- | 486.2 | |
| | methoxy-2-methyl-1H-indol-3-yl}methylidene)-3- | | |
| | oxo-2,3-dihydro-1-benzofuran-5-yl]-3-methylurea | | |
| 599 | 1-{(2Z)-2-[(1-{3-[4-(2-hydroxyethyl)piperazin-1- | 548.3 | |
| | yl]propyl}-5-methoxy-2-methyl-1H-indol-3- | | |
| | yl)methylidene]-3-oxo-2,3-dihydro-1-benzofuran-5- | | |
| | yl}-3-methylurea | | |
| 600 | 5-methoxy-N,N-dimethyl-3-[(Z)-{5- | (FTMS) | |
| | [(methylcarbamoyl)amino]-3-oxo-1-benzofuran- | 435.16551 | |
| | 2(3H)-ylidene}methyl]-1H-indole-2-carboxamide | | |
| 601 | 1-[(2Z)-2-({5-methoxy-2-[(4-methylpiperazin-1- | (FTMS) | |
| | yl)carbonyl]-1H-indol-3-yl}methylidene)-3-oxo-2,3- | 490.20749 | |
| | dihydro-1-benzofuran-5-yl]-3-methylurea | | |
| 602 | N-[2-(dimethylamino)ethyl]-5-methoxy-N-methyl-3- | (FTMS) | |
| | [(Z)-{5-[(methylcarbamoyl)amino]-3-oxo-1- | 492.22303 | |
| | benzofuran-2(3H)-ylidene}methyl]-1H-indole-2- | | |
| | carboxamide | | |
| 603 | 5-methoxy-N-methyl-3-[(Z)-{5- | (FTMS) | |
| | [(methylcarbamoyl)amino]-3-oxo-1-benzofuran- | 421.15015 | |
| | 2(3H)-ylidene}methyl]-1H-indole-2-carboxamide | | |
| 604 | 1-{(2Z)-2-[(2-cyano-5-methoxy-1H-indol-3- | 389.2 | |
| | yl)methylidene]-3-oxo-2,3-dihydro-1-benzofuran-5- | | |
| | yl}-3-methylurea | | |
| 605 | N-[2-(dimethylamino)ethyl]-5-methoxy-3-[(Z)-{5- | 478.1 | |
| | [(methylcarbamoyl)amino]-3-oxo-1-benzofuran- | | |
| | 2(3H)-ylidene}methyl]-1H-indole-2-carboxamide | | |
| 606 | 1-[(2Z)-2-{[5-methoxy-2-(1,2,3,6-tetrahydropyridin- | 445.2 | |
| | 4-yl)-1H-indol-3-yl]methylidene}-3-oxo-2,3-dihydro- | | |
| | 1-benzofuran-5-yl]-3-methylurea | | |
| 607 | 1-[(2Z)-2-({5-methoxy-1-[2-(4-methylpiperazin-1- | 571.3 | |
| | yl)ethyl]-2-(1,2,3,6-tetrahydropyridin-4-yl)-1H-indol- | | |
| | 3-yl}methylidene)-3-oxo-2,3-dihydro-1-benzofuran- | | |
| | 5-yl]-3-methylurea | | |
| 608 | 1-[(2Z)-2-{[5-methoxy-2-methyl-1-(2-morpholin-4- | 491.1 | |
| | ylethyl)-1H-indol-3-yl]methylidene}-3-oxo-2,3- | | |
| | dihydro-1-benzofuran-5-yl]-3-methylurea | | |
| 609 | 1-[(2Z)-2-{[5-methoxy-2-methyl-1-(2-piperidin-1- | 489.3 | |
| | ylethyl)-1H-indol-3-yl]methylidene}-3-oxo-2,3- | | |
| | dihydro-1-benzofuran-5-yl]-3-methylurea | | |
| 610 | 1-[(2Z)-2-({1-[2-(dimethylamino)ethyl]-5-methoxy-2- | 449.3 | |
| | methyl-1H-indol-3-yl}methylidene)-3-oxo-2,3- | | |
| | dihydro-1-benzofuran-5-yl]-3-methylurea | | |
| 611 | 1-[(2Z)-2-{[5-methoxy-2-methyl-1-(2-pyrrolidin-1- | 475.2 | |
| | ylethyl)-1H-indol-3-yl]methylidene}-3-oxo-2,3- | | |
| | dihydro-1-benzofuran-5-yl]-3-methylurea | | |
| 612 | 1-[(2Z)-2-({2-[(dimethylamino)methyl]-5-methoxy- | (FTMS) | |
| | 1H-indol-3-yl}methylidene)-3-oxo-2,3-dihydro-1- | 421.18618 | |
| | benzofuran-5-yl]-3-methylurea | | |
| 613 | 1-[(2Z)-2-{[2-({[2- | (FTMS) | |
| | (dimethylamino)ethyl](methyl)amino}methyl)-5- | 478.2434 | |
| | methoxy-1Hindol-3-yl]methylidene}-3-oxo-2,3- | | |
| | dihydro-1-benzofuran-5-yl]-3-methylurea | | |
| 614 | 1-[(2Z)-2-({5-methoxy-2-[3-(1-methylethyl)-1,2,4- | (FTMS) | |
| | oxadiazol-5-yl]-1-[2-(4-methylpiperazin-1-yl)ethyl]- | 600.29233 | |
| | 1H-indol-3-yl}methylidene)-3-oxo-2,3-dihydro-1- | | |
| | benzofuran-5-yl]-3-methylurea | | |
| 615 | 1-[(2Z)-2-({5-methoxy-2-[(4-methylpiperazin-1- | (FTMS) | |
| | yl)methyl]-1H-indol-3-yl}methylidene)-3-oxo-2,3- | 476.22819 | |
| | dihydro-1-benzofuran-5-yl]-3-methylurea | | |
| 616 | 1-[(2Z)-2-{[5-methoxy-2-(1,3,5-trimethyl-1H- | 472.2 | |
| | pyrazol-4-yl)-1H-indol-3-yl]methylidene}-3-oxo-2,3- | | |
| | dihydro-1-benzofuran-5-yl]-3-methylurea | | |
| 617 | 1-[(2Z)-2-({5-methoxy-2-methyl-1-[2-(4- | 567.3 | |
| | methylpiperazin-1-yl)ethyl]-1H-indol-3- | | |
| | yl}methylidene)-3-oxo-2,3-dihydro-1-benzofuran-5- | | |
| | yl]-3-pyridin-3-ylurea | | |
| 618 | 1-[(2Z)-2-({5-methoxy-2-methyl-1-[2-(4- | 692.3 | |
| | methylpiperazin-1-yl)ethyl]-1H-indol-3- | | |
| | yl}methylene)-3-oxo-2,3-dihydro-1-benzofuran-5- | | |
| | yl]-3-{4-[(4-methylpiperazin-1- | | |
| | yl)carbonyl]phenyl}urea | | |
| 619 | 1-[(2Z)-2-{[1-(2-hydroxyethyl)-5-methoxy-2-methyl- | 610 | |
| | 1H-indol-3-yl]methylene}-3-oxo-2,3-dihydro-1- | | |
| | benzofuran-5-yl]-3-{4-[(4-methylpiperazin-1- | | |
| | yl)carbonyl]phenyl}urea | | |
| 620 | 1-{(2Z)-2-[(1-ethyl-5-methoxy-2-methyl-1H-indol-3- | 595 | |
| | yl)methylene]-3-oxo-2,3-dihydro-1-benzofuran-5- | | |
| | yl}-3-{4-[(4-methylpiperazin-1- | | |
| | yl)carbonyl]phenyl}urea | | |
| 621 | 1-[(2Z)-2-({5-methoxy-2-[3-(1-methylethyl)-1,2,4- | | calcd for |
| | oxadiazol-5-yl]-1-[2-(4-methylpiperazin-1-yl)ethyl]- | | C32H37N7O5 + H+, |
| | 1H-indol-3-yl}methylidene)-3-oxo-2,3-dihydro-1- | | 600.29289 |
| | benzofuran-5-yl]-3-methylurea | | found |
| | | | 600.29233 |
| 622 | N-(2-hydroxy-1,1-dimethylethyl)-5-methoxy-3-[(Z)- | | calcd for |
| | {5-[(methylcarbamoyl)amino]-3-oxo-1-benzofuran- | | C25H26N4O6 + H+, |
| | 2(3H)-ylidene}methyl]-1H-indole-2-carboxamide | | 479.19251 |
| | | | found |
| | | | 479.19232 |
| 623 | 1-[(2Z)-2-({2-(3,5-dimethylisoxazol-4-yl)-5- | | calcd for |
| | methoxy-1-[2-(4-methylpiperazin-1-yl)ethyl]-1H- | | C32H36N6O5 + H+, |
| | indol-3-yl}methylidene)-3-oxo-2,3-dihydro-1- | | 585.28200 |
| | benzofuran-5-yl]-3-methylurea | | found |
| | | | 585.28032 |
| 624 | 1-{(2Z)-2-[(2-{4-[(dimethylamino)methyl]phenyl}-5- | 497.3 | |
| | methoxy-1H-indol-3-yl)methylidene]-3-oxo-2,3- | | |
| | dihydro-1-benzofuran-5-yl}-3-methylurea | | |
| 625 | 1-[(2Z)-2-{[2-(3,5-dimethyl-1H-pyrazol-4-yl)-5- | | calcd for |
| | methoxy-1H-indol-3-yl]methylidene}-3-oxo-2,3- | | C25H23N5O4 + H+, |
| | dihydro-1-benzofuran-5-yl]-3-methylurea | | 458.18228 |
| | | | found |
| | | | 458.18179 |
| 626 | 1-[(2Z)-2-({2-(3-cyclopropyl-1,2,4-oxadiazol-5-yl)-5- | | calcd for |
| | methoxy-1-[2-(4-methylpiperazin-1-yl)ethyl]-1H- | | C32H35N7O5 + H+, |
| | indol-3-yl}methylidene)-3-oxo-2,3-dihydro-1- | | 598.27724 |
| | benzofuran-5-yl]-3-methylurea | | found |
| | | | 598.27635 |
| 627 | 1-[(2Z)-2-{[5-methoxy-2-(1,3,5-trimethyl-1H- | 472.2 257.1 | calcd for |
| | pyrazol-4-yl)-1H-indol-3-yl]methylidene}-3-oxo-2,3- | | C26H25N5O4 + H+, |
| | dihydro-1-benzofuran-5-yl]-3-methylurea | | 472.19793 |
| | | | found 472.19776 |
| 628 | 1-[(2Z)-2-({2-cyano-5-methoxy-1-[2-(4- | | calcd for |
| | methylpiperazin-1-yl)ethyl]-1H-indol-3- | | C28H30N6O4 + H+, |
| | yl}methylidene)-3-oxo-2,3-dihydro-1-benzofuran-5- | | 515.24013 |
| | yl]-3-methylurea | | found 515.23949 |
| 629 | 1-{(2Z)-2-[(2-{3-[(dimethylamino)methyl]phenyl}-5- | 497.3 | |
| | methoxy-1H-indol-3-yl)methylidene]-3-oxo-2,3- | | |
| | dihydro-1-benzofuran-5-yl}-3-methylurea | | |
| 630 | 1-{(2Z)-2-[(2-{4-[(dimethylamino)methyl]phenyl}-5- | 312.2 | |
| | methoxy-1-[2-(4-methylpiperazin-1-yl)ethyl]-1H- | | |
| | indol-3-yl)methylidene]-3-oxo-2,3-dihydro-1- | | |
| | benzofuran-5-yl}-3-methylurea | | |
| 631 | 1-{(2Z)-2-[(2-tert-butyl-5-methoxy-1H-indol-3- | | calcd for |
| | yl)methylidene]-3-oxo-2,3-dihydro-1-benzofuran-5- | | C24H25N3O4 + H+, |
| | yl}-3-methylurea | | 420.19178 |
| | | | found 420.19075 |
| 632 | 1-[(2Z)-2-({5-methoxy-2-methyl-1-[2-(4- | 567.3 284.2 | |
| | methylpiperazin-1-yl)ethyl]-1H-indol-3- | 304.7 | |
| | yl}methylidene)-3-oxo-2,3-dihydro-1-benzofuran-5- | | |
| | yl]-3-pyridin-3-ylurea | | |
| 633 | 1-[4-(dimethylamino)phenyl]-3-[(2Z)-2-({5-methoxy- | 609.4 325.7 | |
| | 2-methyl-1-[2-(4-methylpiperazin-1-yl)ethyl]-1H- | 305.2 | |
| | indol-3-yl}methylidene)-3-oxo-2,3-dihydro-1- | | |
| | benzofuran-5-yl]urea | | |
| 634 | 1-[(2Z)-2-{[1-(3-cyanopropyl)-5-methoxy-2-(1,3,5- | | calcd for |
| | trimethyl-1H-pyrazol-4-yl)-1H-indol-3- | | C30H30N6O4 + H+, |
| | yl]methylene}-3-oxo-2,3-dihydro-1-benzofuran-5- | | 539.24013 |
| | yl]-3-methylurea | | found 539.23879 |
| 635 | 1-[(2Z)-2-{[5-methoxy-2-(1,3,5-trimethyl-1H- | | calcd for |
| | pyrazol-4-yl)-1H-indol-3-yl]methylene}-3-oxo-2,3- | | C30H26N6O4 + H+, |
| | dihydro-1-benzofuran-5-yl]-3-pyridin-3-ylurea | | 535.20883 |
| | | | found 535.20761 |
| 636 | 1-[(2Z)-2-({5-methoxy-2-(4-methylpiperazin-1-yl)-1- | | calcd for |
| | [2-(4-methylpiperazin-1-yl)ethyl]-1H-indol-3- | | C32H41N7O4 + H+, |
| | yl}methylene)-3-oxo-2,3-dihydro-1-benzofuran-5- | | 588.32928 |
| | yl]-3-methylurea | | found 588.32872 |
| 637 | 1-[(2Z)-2-{[2-(2,6-dimethoxyphenyl)-5-methoxy-1H- | | calcd for |
| | indol-3-yl]methylene}-3-oxo-2,3-dihydro-1- | | C28H25N3O6 + H+, |
| | benzofuran-5-yl]-3-methylurea | | 500.18161 |
| | | | found 500.18133 |
| 638 | 1-[(2Z)-2-({2-cyclopentyl-5-methoxy-1-[2-(4- | 311.2 331.7 | |
| | methylpiperazin-1-yl)ethyl]-1H-indol-3- | | |
| | yl}methylene)-3-oxo-2,3-dihydro-1-benzofuran-5- | | |
| | yl]-3-pyridin-3-ylurea | | |
| 639 | N-[2-(dimethylamino)ethyl]-4-[({(2Z)-2-[(1-ethyl-5- | 596.2 298.6 | |
| | methoxy-2-methyl-1H-indol-3-yl)methylene]-3-oxo- | | |
| | 2,3-dihydro-1-benzofuran-5-yl}carbamoyl)amino]- | | |
| | N-methylbenzamide | | |
| 640 | 1-[(2Z)-2-({5-methoxy-2-methyl-1-[2-(4- | 518.3 280.1 | |
| | methylpiperazin-1-yl)ethyl]-1H-indol-3- | | |
| | yl}methylene)-6-methyl-3-oxo-2,3-dihydro-1- | | |
| | benzofuran-5-yl]-3-methylurea | | |
| 641 | 1-{(2Z)-2-[(2-cyclohexyl-5-methoxy-1H-indol-3- | 446.2 | |
| | yl)methylene]-3-oxo-2,3-dihydro-1-benzofuran-5- | | |
| | yl}-3-methylurea | | |
| 642 | 1-{(2Z)-2-[(1-ethyl-5-methoxy-1H-indol-3- | 580.3 | |
| | yl)methylene]-3-oxo-2,3-dihydro-1-benzofuran-5- | | |
| | yl}-3-{4-[(4-methylpiperazin-1- | | |
| | yl)carbonyl]phenyl}urea | | |
| 643 | 1-{(2Z)-2-[(5-methoxy-1H-indol-3-yl)methylene]-3- | 552.3 | |
| | oxo-2,3-dihydro-1-benzofuran-5-yl}-3-{4-[(4- | | |
| | methylpiperazin-1-yl)carbonyl]phenyl}urea | | |
| 644 | 1-{(2Z)-2-[(2-cyclohexyl-5-methoxy-1H-indol-3- | 634.3 338.2 | |
| | yl)methylene]-3-oxo-2,3-dihydro-1-benzofuran-5- | | |
| | yl}-3-{4-[(4-methylpiperazin-1- | | |
| | yl)carbonyl]phenyl}urea | | |
| 645 | 1-{(2Z)-2-[(2-cyclohexyl-1-ethyl-5-methoxy-1H- | 662.4 | |
| | indol-3-yl)methylene]-3-oxo-2,3-dihydro-1- | | |
| | benzofuran-5-yl}-3-{4-[(4-methylpiperazin-1- | | |
| | yl)carbonyl]phenyl}urea | | |
| 646 | 1-[(2Z)-2-({5-methoxy-1-[2-(4-methylpiperazin-1- | | calcd for |
| | yl)ethyl]-2-(1,3,5-trimethyl-1H-pyrazol-4-yl)-1H- | | C33H39N7O4 + H+, |
| | indol-3-yl}methylene)-3-oxo-2,3-dihydro-1- | | 598.31363 |
| | benzofuran-5-yl]-3-methylurea | | found 598.31277 |
| 647 | 1-[(2Z)-2-({5-methoxy-2-methyl-1-[2-(4- | 520.3 | |
| | methylpiperazin-1-yl)ethyl]-1H-indol-3- | | |
| | yl}methylene)-3-oxo-2,3-dihydro-1-benzofuran-5- | | |
| | yl]-3-methylthiourea | | |
| 648 | 1-[(2Z)-2-{[1-{2-[(2-hydroxyethyl)amino]ethyl}-5- | | calcd for |
| | methoxy-2-(1,3,5-trimethyl-1H-pyrazol-4-yl)-1H- | | C30H34N6O5 + H+, |
| | indol-3-yl]methylene}-3-oxo-2,3-dihydro-1- | | 559.26635 |
| | benzofuran-5-yl]-3-methylurea | | found 559.2656 |
| 649 | N-[2-(dimethylamino)ethyl]-4-({[(2Z)-2-({5-methoxy- | 694.4 347.7 | |
| | 2-methyl-1-[2-(4-methylpiperazin-1-yl)ethyl]-1H- | | |
| | indol-3-yl}methylene)-3-oxo-2,3-dihydro-1- | | |
| | benzofuran-5-yl]carbamoyl}amino)-N- | | |
| | methylbenzamide | | |
| 650 | 1-(4-{[4-(dimethylamino)piperidin-1- | 622.3 311.7 | |
| | yl]carbonyl}phenyl)-3-{(2Z)-2-[(1-ethyl-5-methoxy- | | |
| | 2-methyl-1H-indol-3-yl)methylene]-3-oxo-2,3- | | |
| | dihydro-1-benzofuran-5-yl}urea | | |
| 651 | 1-{(2Z)-2-[(1-ethyl-5-methoxy-2-methyl-1H-indol-3- | 593.3 | |
| | yl)methylene]-3-oxo-2,3-dihydro-1-benzofuran-5- | | |
| | yl}-3-{4-[(1-methylpiperidin-4- | | |
| | yl)carbonyl]phenyl}urea | | |
| 652 | 1-(4-{[2- | 568.3 284.7 | |
| | (dimethylamino)ethyl](methyl)amino}phenyl)-3- | | |
| | {(2Z)-2-[(1-ethyl-5-methoxy-2-methyl-1H-indol-3- | | |
| | yl)methylene]-3-oxo-2,3-dihydro-1-benzofuran-5- | | |
| | yl}urea | | |
| 653 | 1-(4-{[3- | 582.3 291.6 | |
| | (dimethylamino)propyl](methyl)amino}phenyl)-3- | | |
| | {(2Z)-2-[(1-ethyl-5-methoxy-2-methyl-1H-indol-3- | | |
| | yl)methylene]-3-oxo-2,3-dihydro-1-benzofuran-5- | | |
| | yl}urea | | |
| 654 | 1-{4-[3-(dimethylamino)propoxy]phenyl}-3-{(2Z)-2- | 569.3 285.1 | |
| | [(1-ethyl-5-methoxy-2-methyl-1H-indol-3- | | |
| | yl)methylene]-3-oxo-2,3-dihydro-1-benzofuran-5- | | |
| | yl}urea | | |
| 655 | N-[2-(dimethylamino)ethyl]-4-[({(2Z)-2-[(1-ethyl-5- | 582.3 | |
| | methoxy-1H-indol-3-yl)methylene]-3-oxo-2,3- | | |
| | dihydro-1-benzofuran-5-yl}carbamoyl)amino]-N- | | |
| | methylbenzamide | | |
| 656 | 1-[(2Z)-2-{[5-methoxy-1-(2-piperazin-1-ylethyl)-2- | | calcd for |
| | (1,3,5-trimethyl-1H-pyrazol-4-yl)-1H-indol-3- | | C32H37N7O4 + H+, |
| | yl]methylene}-3-oxo-2,3-dihydro-1-benzofuran-5- | | 584.29798 |
| | yl]-3-methylurea | | found 584.29697 |
| 657 | N-[3-(dimethylamino)propyl]-4-[({(2Z)-2-[(1-ethyl-5- | 610.3 | |
| | methoxy-2-methyl-1H-indol-3-yl)methylene]-3-oxo- | | |
| | 2,3-dihydro-1-benzofuran-5-yl}carbamoyl)amino]- | | |
| | N-methylbenzamide | | |
| 658 | 1-{(2Z)-2-[(5-methoxy-1,2-dimethyl-1H-indol-3- | 580.4 | |
| | yl)methylidene]-3-oxo-2,3-dihydro-1-benzofuran-5- | | |
| | yl}-3-{4-[(4-methylpiperazin-1- | | |
| | yl)carbonyl]phenyl}urea | | |
| 659 | 4-[({(2Z)-2-[(1-ethyl-5-methoxy-2-methyl-1H-indol- | 568.3 | |
| | 3-yl)methylene]-3-oxo-2,3-dihydro-1-benzofuran-5- | | |
| | yl}carbamoyl)amino]-N-[2- | | |
| | (methylamino)ethyl]benzamide | | |
| 660 | 4-[({(2Z)-2-[(2-cyclohexyl-5-methoxy-1H-indol-3- | 608.3 | |
| | yl)methylene]-3-oxo-2,3-dihydro-1-benzofuran-5- | | |
| | yl}carbamoyl)amino]-N-[2- | | |
| | (methylamino)ethyl]benzamide | | |
| 661 | 4-[({(2Z)-2-[(5-methoxy-1,2-dimethyl-1H-indol-3- | 554.3 | |
| | yl)methylene]-3-oxo-2,3-dihydro-1-benzofuran-5- | | |
| | yl}carbamoyl)amino]-N-[2- | | |
| | (methylamino)ethyl]benzamide | | |
| 662 | 1-[(2Z)-2-({5-methoxy-2-methyl-1-[2-(4- | 518.3 280.2 | |
| | methylpiperazin-1-yl)ethyl]-1H-indol-3- | | |
| | yl}methylidene)-7-methyl-3-oxo-2,3-dihydro-1- | | |
| | benzofuran-5-yl]-3-methylurea | | |
| 663 | 1-{4-[4-(dimethylamino)butyl]phenyl}-3-{(2Z)-2-[(1- | 567.3 | |
| | ethyl-5-methoxy-2-methyl-1H-indol-3- | | |
| | yl)methylidene]-3-oxo-2,3-dihydro-1-benzofuran-5- | | |
| | yl}urea | | |
| 664 | 1-[(2Z)-2-{[1-(2-{[2- | 586.31248 | calcd for |
| | (dimethylamino)ethyl]amino}ethyl)-5-methoxy-2- | | C32H39N7O4 + H+, |
| | (1,3,5-trimethyl-1H-pyrazol-4-yl)-1H-indol-3- | | 586.31363 |
| | yl]methylidene}-3-oxo-2,3-dihydro-1-benzofuran-5- | | found 586.31248 |
| | yl]-3-methylurea | | |
| 665 | 1-[(2Z)-2-{[1-(2-{[2- | 600.32803 | calcd for |
| | (dimethylamino)ethyl](methyl)amino}ethyl)-5- | | C33H41N7O4 + H+, |
| | methoxy-2-(1,3,5-trimethyl-1H-pyrazol-4-yl)-1H- | | 600.32928 |
| | indol-3-yl]methylidene}-3-oxo-2,3-dihydro-1- | | found 600.32803 |
| | benzofuran-5-yl]-3-methylurea | | |
| 666 | N-[3-(dimethylamino)propyl]-4-[({(2Z)-2-[(1-ethyl-5- | 596.2 298.6 | |
| | methoxy-2-methyl-1H-indol-3-yl)methylidene]-3- | | |
| | oxo-2,3-dihydro-1-benzofuran-5- | | |
| | yl}carbamoyl)amino]benzamide | | |
| 667 | 1-{4-[2-(dimethylamino)ethoxy]phenyl}-3-{(2Z)-2- | 555.2 278.1 | |
| | [(1-ethyl-5-methoxy-2-methyl-1H-indol-3- | | |
| | yl)methylidene]-3-oxo-2,3-dihydro-1-benzofuran-5- | | |
| | yl}urea | | |
| 668 | 1-{4-[4-(dimethylamino)butoxy]phenyl}-3-{(2Z)-2- | 583.3 | |
| | [(1-ethyl-5-methoxy-2-methyl-1H-indol-3- | | |
| | yl)methylidene]-3-oxo-2,3-dihydro-1-benzofuran-5- | | |
| | yl}urea | | |
| 669 | 4-({[(2Z)-2-({5-methoxy-2-methyl-1-[2-(4- | 666.3 333.7 | |
| | methylpiperazin-1-yl)ethyl]-1H-indol-3- | | |
| | yl}methylidene)-3-oxo-2,3-dihydro-1-benzofuran-5- | | |
| | yl]carbamoyl}amino)-N-[2- | | |
| | (methylamino)ethyl]benzamide | | |
| 670 | 1-{4-[2-(dimethylamino)ethyl]phenyl}-3-{(2Z)-2-[(1- | 539.3 | |
| | ethyl-5-methoxy-2-methyl-1H-indol-3- | | |
| | yl)methylidene]-3-oxo-2,3-dihydro-1-benzofuran-5- | | |
| | yl}urea | | |
| 671 | 1-{4-[3-(dimethylamino)propyl]phenyl}-3-{(2Z)-2- | 553.2 277.1 | |
| | [(1-ethyl-5-methoxy-2-methyl-1H-indol-3- | | |
| | yl)methylidene]-3-oxo-2,3-dihydro-1-benzofuran-5- | | |
| | yl}urea | | |
| 672 | 1-[(2Z)-2-({5-methoxy-1-[2-(methylamino)ethyl]-2- | | calcd for |
| | (1,3,5-trimethyl-1H-pyrazol-4-yl)-1H-indol-3- | | C29H32N6O4 + H+, |
| | yl}methylidene)-3-oxo-2,3-dihydro-1-benzofuran-5- | | 529.25578 |
| | yl]-3-methylurea | | found 529.2548 |
| 673 | 1-[(2Z)-2-({1-[2-(dimethylamino)ethyl]-5-methoxy-2- | | calcd for |
| | (1,3,5-trimethyl-1H-pyrazol-4-yl)-1H-indol-3- | | C30H34N6O4 + H+, |
| | yl}methylidene)-3-oxo-2,3-dihydro-1-benzofuran-5- | | 543.27143 |
| | yl]-3-methylurea | | found 543.27052 |
| 674 | 1-{4-[4-(dimethylamino)butoxy]phenyl}-3-[(2Z)-2- | 681.4 341.2 | |
| | ({5-methoxy-2-methyl-1-[2-(4-methylpiperazin-1- | 241.5 | |
| | yl)ethyl]-1H-indol-3-yl}methylidene)-3-oxo-2,3- | | |
| | dihydro-1-benzofuran-5-yl]urea | | |
| 675 | 1-(4-{[4- | 596.3 | |
| | (dimethylamino)butyl](methyl)amino}phenyl)-3- | | |
| | {(2Z)-2-[(1-ethyl-5-methoxy-2-methyl-1H-indol-3- | | |
| | yl)methylidene]-3-oxo-2,3-dihydro-1-benzofuran-5- | | |
| | yl}urea | | |
| 676 | N-{4-[({(2Z)-2-[(1-ethyl-5-methoxy-2-methyl-1H- | 582.3 291.7 | |
| | indol-3-yl)methylidene]-3-oxo-2,3-dihydro-1- | | |
| | benzofuran-5-yl}carbamoyl)amino]phenyl}-N3,N3- | | |
| | dimethyl-b-alaninamide | | |
| 677 | 1-{(2Z)-2-[(1-ethyl-5-methoxy-2-methyl-1H-indol-3- | 630.3 315.6 | |
| | yl)methylidene]-3-oxo-2,3-dihydro-1-benzofuran-5- | | |
| | yl}-3-{4-[(4-methylpiperazin-1- | | |
| | yl)sulfonyl]phenyl}urea | | |
| 678 | 1-(4-{[2-(dimethylamino)ethyl]amino}phenyl)-3- | 554.3 277.6 | |
| | {(2Z)-2-[(1-ethyl-5-methoxy-2-methyl-1H-indol-3- | | |
| | yl)methylidene]-3-oxo-2,3-dihydro-1-benzofuran-5- | | |
| | yl}urea | | |
| 679 | N-[2-(dimethylamino)ethyl]-4-[({(2Z)-2-[(1-ethyl-5- | 632.2 316.6 | |
| | methoxy-2-methyl-1H-indol-3-yl)methylidene]-3- | | |
| | oxo-2,3-dihydro-1-benzofuran-5- | | |
| | yl}carbamoyl)amino]-N-methylbenzenesulfonamide | | |
| 680 | 1-[(2Z)-2-{[5-(2-methoxyethoxy)-2-(1,3,5-trimethyl- | | calcd for |
| | 1H-pyrazol-4-yl)-1H-indol-3-yl]methylidene}-3-oxo- | | C28H29N5O5 + H+, |
| | 2,3-dihydro-1-benzofuran-5-yl]-3-methylurea | | 516.22415 |
| | | | found (ESI, |
| | | | [M + H]+ Obs'd), |
| | | | 516.2239 calcd |
| | | | for |
| | | | C28H29N5O5 + H+, |
| | | | 516.22415 |
| | | | found (ESI, |
| | | | [M + H]+ Calc'd), |
| | | | 516.2242 |
| 681 | 1-[4-(dimethylamino)phenyl]-3-{(2Z)-2-[(1-ethyl-5- | 511.2 256.1 | |
| | methoxy-2-methyl-1H-indol-3-yl)methylidene]-3- | | |
| | oxo-2,3-dihydro-1-benzofuran-5-yl}urea | | |
| 682 | N-[2-(dimethylamino)ethyl]-4-({[(2Z)-2-{[5-methoxy- | 662.4 331.7 | |
| | 2-(1,3,5-trimethyl-1H-pyrazol-4-yl)-1H-indol-3- | | |
| | yl]methylidene}-3-oxo-2,3-dihydro-1-benzofuran-5- | | |
| | yl]carbamoyl}amino)-N-methylbenzamide | | |
| 683 | 1-{(2Z)-2-[(1-ethyl-5-methoxy-2-methyl-1H-indol-3- | 555.3 278.2 | |
| | yl)methylidene]-3-oxo-2,3-dihydro-1-benzofuran-5- | | |
| | yl}-3-{4-[3-(methylamino)propoxy]phenyl}urea | | |
| 684 | 1-(4-{[2-(dimethylamino)ethyl]amino}phenyl)-3- | 652.4 326.7 | |
| | [(2Z)-2-({5-methoxy-2-methyl-1-[2-(4- | 231.8 | |
| | methylpiperazin-1-yl)ethyl]-1H-indol-3- | | |
| | yl}methylidene)-3-oxo-2,3-dihydro-1-benzofuran-5- | | |
| | yl]urea | | |
| 685 | N-[4-({[(2Z)-2-({5-methoxy-2-methyl-1-[2-(4- | 680.3 340.7 | |
| | methylpiperazin-1-yl)ethyl]-1H-indol-3- | 241.1 | |
| | yl}methylidene)-3-oxo-2,3-dihydro-1-benzofuran-5- | | |
| | yl]carbamoyl}amino)phenyl]-N3,N3-dimethyl-b- | | |
| | alaninamide | | |
| 686 | 1-{(2Z)-2-[(1-ethyl-5-methoxy-2-methyl-1H-indol-3- | 581.1 | |
| | yl)methylidene]-3-oxo-2,3-dihydro-1-benzofuran-5- | | |
| | yl}-3-[4-(morpholin-4-ylcarbonyl)phenyl]urea | | |
| 687 | 1-[(2Z)-2-({5-methoxy-2-methyl-1-[2-(4- | 679.1 340.1 | |
| | methylpiperazin-1-yl)ethyl]-1H-indol-3- | | |
| | yl}methylidene)-3-oxo-2,3-dihydro-1-benzofuran-5- | | |
| | yl]-3-[4-(morpholin-4-ylcarbonyl)phenyl]urea | | |
| 688 | 4-[({(2Z)-2-[(2-cyclohexyl-5-methoxy-1H-indol-3- | 636.4 318.7 | |
| | yl)methylidene]-3-oxo-2,3-dihydro-1-benzofuran-5- | | |
| | yl}carbamoyl)amino]-N-[2-(dimethylamino)ethyl]-N- | | |
| | methylbenzamide | | |
| 689 | 1-[(2Z)-2-{[5-methoxy-2-(1,3,5-trimethyl-1H- | 660.1 351.1 | |
| | pyrazol-4-yl)-1H-indol-3-yl]methylidene}-3-oxo-2,3- | 330.5 | |
| | dihydro-1-benzofuran-5-yl]-3-{4-[(4- | | |
| | methylpiperazin-1-yl)carbonyl]phenyl}urea | | |
| 690 | 1-[(2Z)-2-{[5-methoxy-2-(1,3,5-trimethyl-1H- | 647.3 324.2 | |
| | pyrazol-4-yl)-1H-indol-3-yl]methylidene}-3-oxo-2,3- | | |
| | dihydro-1-benzofuran-5-yl]-3-[4-(morpholin-4- | | |
| | ylcarbonyl)phenyl]urea | | |
| 691 | N-[4-({[(2Z)-2-{[5-methoxy-2-(1,3,5-trimethyl-1H- | 648.1 324.6 | |
| | pyrazol-4-yl)-1H-indol-3-yl]methylidene}-3-oxo-2,3- | | |
| | dihydro-1-benzofuran-5- | | |
| | yl]carbamoyl}amino)phenyl]-N3,N3-dimethyl-b- | | |
| | alaninamide | | |
| 692 | 1-{(2Z)-2-[(2-bromo-5-methoxy-1H-indol-3- | | calcd for |
| | yl)methylidene]-3-oxo-2,3-dihydro-1-benzofuran-5- | | C20H16BrN3O4 + H+, |
| | yl}-3-methylurea | | 442.03969 |
| | | | found 442.03949 |
| 693 | 1-[(2Z)-2-{[7-fluoro-5-methoxy-2-(1,3,5-trimethyl- | | calcd for |
| | 1H-pyrazol-4-yl)-1H-indol-3-yl]methylidene}-3-oxo- | | C26H24FN5O4 + H+, |
| | 2,3-dihydro-1-benzofuran-5-yl]-3-methylurea | | 490.18851 |
| | | | found 490.1879 |
| 694 | N-{4-[({(2Z)-2-[(1-ethyl-5-methoxy-2-methyl-1H- | 596.2 298.6 | |
| | indol-3-yl)methylidene]-3-oxo-2,3-dihydro-1- | | |
| | benzofuran-5-yl}carbamoyl)amino]phenyl}- | | |
| | N,N3,N3-trimethyl-b-alaninamide | | |
| 695 | N-[4-({[(2Z)-2-({5-methoxy-2-methyl-1-[2-(4- | 347.7 | |
| | methylpiperazin-1-yl)ethyl]-1H-indol-3- | | |
| | yl}methylidene)-3-oxo-2,3-dihydro-1-benzofuran-5- | | |
| | yl]carbamoyl}amino)phenyl]-N,N3,N3-trimethyl-b- | | |
| | alaninamide | | |
| 696 | N-(4-{[(2-{[5-methoxy-2-(1,3,5-trimethyl-1H- | 662.4 331.7 | |
| | pyrazol-4-yl)-1H-indol-3-yl]methylidene}-3-oxo-2,3- | | |
| | dihydro-1-benzofuran-5- | | |
| | yl)carbamoyl]amino}phenyl)-N,N3,N3-trimethyl-b- | | |
| | alaninamide | | |
| 697 | 1-(4-{[3-(dimethylamino)pyrrolidin-1- | 674.1 337.5 | |
| | yl]carbonyl}phenyl)-3-[(2Z)-2-{[5-methoxy-2-(1,3,5- | | |
| | trimethyl-1H-pyrazol-4-yl)-1H-indol-3- | | |
| | yl]methylidene}-3-oxo-2,3-dihydro-1-benzofuran-5- | | |
| | yl]urea | | |
| 698 | 4-({[(2Z)-2-{[5-methoxy-2-(1,3,5-trimethyl-1H- | 674.2 337.6 | |
| | pyrazol-4-yl)-1H-indol-3-yl]methylidene}-3-oxo-2,3- | 358.1 | |
| | dihydro-1-benzofuran-5-yl]carbamoyl}amino)-N- | | |
| | methyl-N-(1-methylpyrrolidin-3-yl)benzamide | | |
| 699 | 1-{4-[(4-ethylpiperazin-1-yl)carbonyl]phenyl}-3- | 674.2 337.6 | |
| | [(2Z)-2-{[5-methoxy-2-(1,3,5-trimethyl-1H-pyrazol- | 358.1 | |
| | 4-yl)-1H-indol-3-yl]methylidene}-3-oxo-2,3-dihydro- | | |
| | 1-benzofuran-5-yl]urea | | |
| 700 | 1-[(2Z)-2-{[5-methoxy-2-(1,3,5-trimethyl-1H- | 688.3 344.6 | |
| | pyrazol-4-yl)-1H-indol-3-yl]methylidene}-3-oxo-2,3- | 365.1 | |
| | dihydro-1-benzofuran-5-yl]-3-(4-{[4-(1- | | |
| | methylethyl)piperazin-1-yl]carbonyl}phenyl)urea | | |
| 701 | 4-({[(2Z)-2-{[5-methoxy-2-(1,3,5-trimethyl-1H- | 688.3 344.7 | |
| | pyrazol-4-yl)-1H-indol-3-yl]methylidene}-3-oxo-2,3- | | |
| | dihydro-1-benzofuran-5-yl]carbamoyl}amino)-N- | | |
| | methyl-N-(2-pyrrolidin-1-ylethyl)benzamide | | |
| 702 | 1-(4-{[4-(dimethylamino)piperidin-1- | 688.5 | |
| | yl]carbonyl}phenyl)-3-[(2Z)-2-{[5-methoxy-2-(1,3,5- | | |
| | trimethyl-1H-pyrazol-4-yl)-1H-indol-3- | | |
| | yl]methylidene}-3-oxo-2,3-dihydro-1-benzofuran-5- | | |
| | yl]urea | | |
| 703 | 1-{4-[(3,4-dimethylpiperazin-1-yl)carbonyl]phenyl}- | 674.3 | |
| | 3-[(2Z)-2-{[5-methoxy-2-(1,3,5-trimethyl-1H- | | |
| | pyrazol-4-yl)-1H-indol-3-yl]methylidene}-3-oxo-2,3- | | |
| | dihydro-1-benzofuran-5-yl]urea | | |
| 704 | 4-({[(2Z)-2-{[5-methoxy-2-(1,3,5-trimethyl-1H- | 605.3 | |
| | pyrazol-4-yl)-1H-indol-3-yl]methylidene}-3-oxo-2,3- | | |
| | dihydro-1-benzofuran-5-yl]carbamoyl}amino)-N,N- | | |
| | dimethylbenzamide | | |
| 705 | 1-{(2Z)-2-[(2-{1-[2-(dimethylamino)ethyl]-3,5- | | calcd for |
| | dimethyl-1H-pyrazol-4-yl}-5-methoxy-1H-indol-3- | | C29H32N6O4 + H+, |
| | yl)methylidene]-3-oxo-2,3-dihydro-1-benzofuran-5- | | 529.25578 |
| | yl}-3-methylurea | | found 529.25566 |
| 706 | 1-[(2Z)-2-({5-methoxy-2-[1-(2-methylpropyl)-1H- | | calcd for |
| | pyrazol-4-yl]-1H-indol-3-yl}methylidene)-3-oxo-2,3- | | C27H27N5O4 + H+, |
| | dihydro-1-benzofuran-5-yl]-3-methylurea | | 486.21358 |
| | | | found 486.21304 |
| 707 | N-[2-(dimethylamino)ethyl]-3-({[(2Z)-2-{[5-methoxy- | 662.3 | |
| | 2-(1,3,5-trimethyl-1H-pyrazol-4-yl)-1H-indol-3- | | |
| | yl]methylidene}-3-oxo-2,3-dihydro-1-benzofuran-5- | | |
| | yl]carbamoyl}amino)-N-methylbenzamide | | |
| 708 | N-[3-(dimethylamino)propyl]-3-({[(2Z)-2-{[5- | 676.3 | |
| | methoxy-2-(1,3,5-trimethyl-1H-pyrazol-4-yl)-1H- | | |
| | indol-3-yl]methylidene}-3-oxo-2,3-dihydro-1- | | |
| | benzofuran-5-yl]carbamoyl}amino)-N- | | |
| | methylbenzamide | | |
| 709 | 1-[(2Z)-2-({5-methoxy-2-[1-(2-methoxyethyl)-3,5- | | calcd for |
| | dimethyl-1H-pyrazol-4-yl]-1H-indol-3- | | C28H29N5O5 + H+, |
| | yl}methylidene)-3-oxo-2,3-dihydro-1-benzofuran-5- | | 516.22415 |
| | yl]-3-methylurea | | found 516.22338 |
| 710 | N-[2-(dimethylamino)ethyl]-4-({[(2Z)-2-{[7-fluoro-5- | 680.3 340.6 | calcd for |
| | methoxy-2-(1,3,5-trimethyl-1H-pyrazol-4-yl)-1H- | | C37H38FN7O5 + H+, |
| | indol-3-yl]methylidene}-3-oxo-2,3-dihydro-1- | | 680.29912; |
| | benzofuran-5-yl]carbamoyl}amino)-N- | | found 680.2984; |
| | methylbenzamide | | |
| 711 | N-[2-(dimethylamino)ethyl]-4-({[(2Z)-2-{[5-methoxy- | 648.3 | |
| | 2-(1,3,5-trimethyl-1H-pyrazol-4-yl)-1H-indol-3- | | |
| | yl]methylidene}-3-oxo-2,3-dihydro-1-benzofuran-5- | | |
| | yl]carbamoyl}amino)benzamide | | |
| 712 | 1-[(2Z)-2-{[6-fluoro-5,7-dimethoxy-2-(1,3,5- | | calcd for |
| | trimethyl-1H-pyrazol-4-yl)-1H-indol-3- | | C27H26FN5O5 + H+, |
| | yl]methylidene}-3-oxo-2,3-dihydro-1-benzofuran-5- | | 520.19907 |
| | yl]-3-methylurea | | found 520.19812 |
| 713 | 1-[(2Z)-2-{[6,7-difluoro-5-methoxy-2-(1,3,5- | | calcd for |
| | trimethyl-1H-pyrazol-4-yl)-1H-indol-3- | | C26H23F2N5O4 + H+, |
| | yl]methylidene}-3-oxo-2,3-dihydro-1-benzofuran-5- | | 508.17909 |
| | yl]-3-methylurea | | found 508.17797 |
| 714 | 1-[(2Z)-2-{[2-(3,5-dimethyl-1H-pyrazol-4-yl)-7- | | calcd for |
| | fluoro-5-methoxy-1H-indol-3-yl]methylidene}-3-oxo- | | C25H22FN5O4 + H+, |
| | 2,3-dihydro-1-benzofuran-5-yl]-3-methylurea | | 476.17286 |
| | | | found 476.17177 |
| 715 | 1-(2-aminoethyl)-3-[(2Z)-2-{[7-fluoro-5-methoxy-2- | 519.2 260.1 | |
| | (1,3,5-trimethyl-1H-pyrazol-4-yl)-1H-indol-3- | | |
| | yl]methylidene}-3-oxo-2,3-dihydro-1-benzofuran-5- | | |
| | yl]urea | | |
| 716 | 1-[2-(dimethylamino)ethyl]-3-[(2Z)-2-{[7-fluoro-5- | 547.2 | |
| | methoxy-2-(1,3,5-trimethyl-1H-pyrazol-4-yl)-1H- | | |
| | indol-3-yl]methylidene}-3-oxo-2,3-dihydro-1- | | |
| | benzofuran-5-yl]urea | | |
| 717 | 1-[(2Z)-2-({7-fluoro-5-methoxy-2-[1-(2- | 504.2 | calcd for |
| | methylpropyl)-1H-pyrazol-4-yl]-1H-indol-3- | | C27H26FN5O4 + H+, |
| | yl}methylidene)-3-oxo-2,3-dihydro-1-benzofuran-5- | | 504.20416 |
| | yl]-3-methylurea | | found 504.20328 |
| 718 | 1-[(2Z)-2-{[7-fluoro-5-methoxy-2-(1-methyl-1H- | | calcd for |
| | pyrazol-4-yl)-1H-indol-3-yl]methylidene}-3-oxo-2,3- | | C24H20FN5O4 + H+, |
| | dihydro-1-benzofuran-5-yl]-3-methylurea | | 462.15721 |
| | | | found 462.15722 |
| 719 | 1-{4-[(dimethylamino)methyl]phenyl}-3-{(2Z)-2-[(1- | 525.2 263.1 | |
| | ethyl-5-methoxy-2-methyl-1H-indol-3- | | |
| | yl)methylidene]-3-oxo-2,3-dihydro-1-benzofuran-5- | | |
| | yl}urea | | |
| 720 | 1-{4-[(1,1-dioxidothiomorpholin-4- | 695.1 | |
| | yl)carbonyl]phenyl}-3-[(2Z)-2-{[5-methoxy-2-(1,3,5- | | |
| | trimethyl-1H-pyrazol-4-yl)-1H-indol-3- | | |
| | yl]methylidene}-3-oxo-2,3-dihydro-1-benzofuran-5- | | |
| | yl]urea | | |
| 721 | 1-[(2Z)-2-{[7-fluoro-5-methoxy-2-(1,3,5-trimethyl- | 708.2 354.6 | |
| | 1H-pyrazol-4-yl)-1H-indol-3-yl]methylidene}-3-oxo- | | |
| | 2,3-dihydro-1-benzofuran-5-yl]-3-(4-{[4-(2- | | |
| | hydroxyethyl)piperazin-1-yl]carbonyl}phenyl)urea | | |
| 722 | 1-[(2Z)-2-{[2-(1,3-dimethyl-1H-pyrazol-4-yl)-5- | | calcd for |
| | methoxy-1H-indol-3-yl]methylidene}-3-oxo-2,3- | | C25H23N5O4 + H+, |
| | dihydro-1-benzofuran-5-yl]-3-methylurea | | 458.18228 |
| | | | found 458.18283 |
| 723 | 1-[(2Z)-2-{[2-(1,5-dimethyl-1H-pyrazol-4-yl)-5- | | calcd for |
| | methoxy-1H-indol-3-yl]methylidene}-3-oxo-2,3- | | C25H23N5O4 + H+, |
| | dihydro-1-benzofuran-5-yl]-3-methylurea | | 458.18228 |
| | | | found 458.18272 |
| 724 | 1-[(2Z)-2-({5-methoxy-2-[1-methyl-4- | 512.1 | calcd for |
| | (trifluoromethyl)-1H-pyrazol-3-yl]-1H-indol-3- | | C25H20F3N5O4 + H+, |
| | yl}methylidene)-3-oxo-2,3-dihydro-1-benzofuran-5- | | 512.15402 |
| | yl]-3-methylurea | | found 512.15479 |
| 725 | 1-[(2Z)-2-{[5-methoxy-7-(trifluoromethyl)-2-(1,3,5- | | calcd for |
| | trimethyl-1H-pyrazol-4-yl)-1H-indol-3- | | C27H24F3N5O4 + H+, |
| | yl]methylidene}-3-oxo-2,3-dihydro-1-benzofuran-5- | | 540.18532 |
| | yl]-3-methylurea | | found 540.18505 |
| 726 | 1-[(2Z)-2-{[5-methoxy-7-methyl-2-(1,3,5-trimethyl- | | calcd for |
| | 1H-pyrazol-4-yl)-1H-indol-3-yl]methylidene}-3-oxo- | | C27H27N5O4 + H+, |
| | 2,3-dihydro-1-benzofuran-5-yl]-3-methylurea | | 486.21358 |
| | | | found 486.21361 |
| 727 | 1-[(2Z)-2-{[7-fluoro-5-methoxy-2-(1,3,5-trimethyl- | 573.6 287.3 | |
| | 1H-pyrazol-4-yl)-1H-indol-3-yl]methylidene}-3-oxo- | 307.8 | |
| | 2,3-dihydro-1-benzofuran-5-yl]-3-(2-pyrrolidin-1- | | |
| | ylethyl)urea | | |
| 728 | 1-[(2Z)-2-{[7-fluoro-5-methoxy-2-(1,3,5-trimethyl- | 533.4 267.2 | |
| | 1H-pyrazol-4-yl)-1H-indol-3-yl]methylidene}-3-oxo- | 287.7 | |
| | 2,3-dihydro-1-benzofuran-5-yl]-3-[2- | | |
| | (methylamino)ethyl]urea | | |
| 729 | 1-(4-{[4-(dimethylamino)piperidin-1- | 706.5 353.8 | |
| | yl]carbonyl}phenyl)-3-[(2Z)-2-{[7-fluoro-5-methoxy- | | |
| | 2-(1,3,5-trimethyl-1H-pyrazol-4-yl)-1H-indol-3- | | |
| | yl]methylidene}-3-oxo-2,3-dihydro-1-benzofuran-5- | | |
| | yl]urea | | |
| 730 | 1-{4-[(3,4-dimethylpiperazin-1-yl)carbonyl]phenyl}- | 692.3 346.7 | |
| | 3-[(2Z)-2-{[7-fluoro-5-methoxy-2-(1,3,5-trimethyl- | 367.2 | |
| | 1H-pyrazol-4-yl)-1H-indol-3-yl]methylidene}-3-oxo- | | |
| | 2,3-dihydro-1-benzofuran-5-yl]urea | | |
| 731 | 1-[(2Z)-2-{[7-fluoro-5-methoxy-2-(1,3,5-trimethyl- | 665.4 333.2 | |
| | 1H-pyrazol-4-yl)-1H-indol-3-yl]methylidene}-3-oxo- | | |
| | 2,3-dihydro-1-benzofuran-5-yl]-3-[4-(morpholin-4- | | |
| | ylcarbonyl)phenyl]urea | | |
| 732 | 1-(4-{[3-(dimethylamino)pyrrolidin-1- | 692.4 346.7 | |
| | yl]carbonyl}phenyl)-3-[(2Z)-2-{[7-fluoro-5-methoxy- | | |
| | 2-(1,3,5-trimethyl-1H-pyrazol-4-yl)-1H-indol-3- | | |
| | yl]methylidene}-3-oxo-2,3-dihydro-1-benzofuran-5- | | |
| | yl]urea | | |
| 733 | N-[2-(dimethylamino)ethyl]-4-({[(2Z)-2-{[7-fluoro-5- | 666.4 333.7 | |
| | methoxy-2-(1,3,5-trimethyl-1H-pyrazol-4-yl)-1H- | | |
| | indol-3-yl]methylidene}-3-oxo-2,3-dihydro-1- | | |
| | benzofuran-5-yl]carbamoyl}amino)benzamide | | |

Other compounds of the invention which are made by the processes described herein include the following:

### (Z)-1-(2-((2-Cyclohexyl-7-fluoro-5-methoxy-1-(2-(4-methylpiperazin-1-yl)ethyl)-1H-indol-3-yl)methylene)-3-oxo-2,3-dihydrobenzofuran-5-yl)-3-methylurea

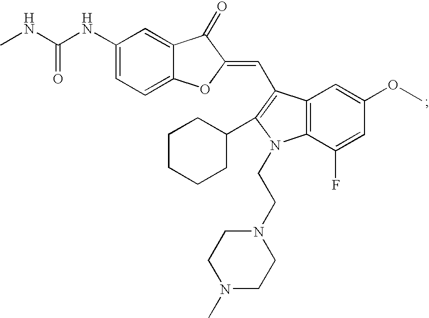

### (Z)-1-(2-((2-Cyclohexyl-7-fluoro-5-methoxy-1H-indol-3-yl)methylene)-3-oxo-2,3-dihydrobenzofuran-5-yl)-3-methylurea

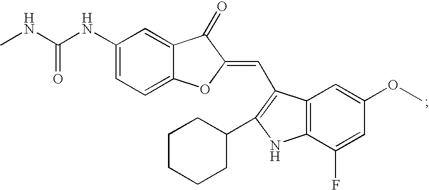

### (Z)-4-(3-(2-((2-Cyclohexyl-7-fluoro-5-methoxy-1H-indol-3-yl)methylene)-3-oxo-2,3-dihydrobenzofuran-5-yl)ureido)-N-(2-(dimethylamino)ethyl)-N-methylbenzamide

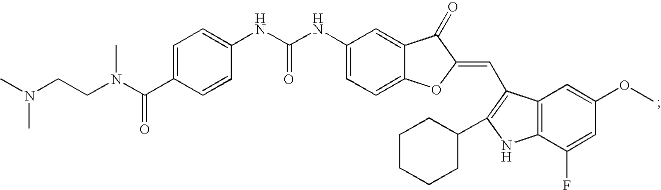

### (Z)-4-(3-(2-((2-Cyclohexyl-7-fluoro-5-methoxy-1H-indol-3-yl)methylene)-3-oxo-2,3-dihydrobenzofuran-5-yl)ureido)-N-(2-(dimethylamino)ethyl)benzamide

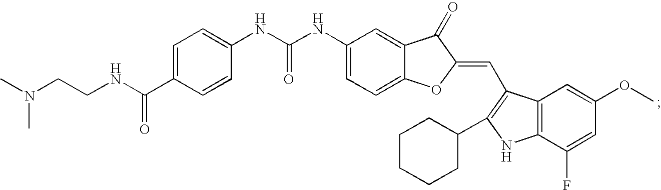

### (Z)-N-(2-(Dimethylamino)ethyl)-4-(3-(2-((7-fluoro-5-methoxy-2-methyl-1H-indol-3-yl)methylene)-3-oxo-2,3-dihydrobenzofuran-5-yl)ureido)-N-methylbenzamide

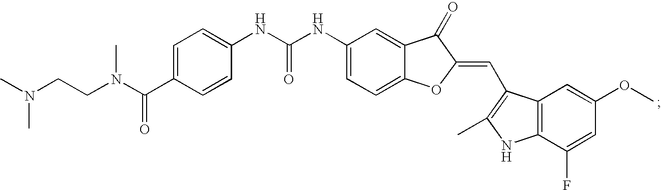

### (Z)-N-(2-(Dimethylamino)ethyl)-4-(3-(2-((7-fluoro-5-methoxy-2-methyl-1H-indol-3-yl)methylene)-3-oxo-2,3-dihydrobenzofuran-5-yl)ureido)benzamide

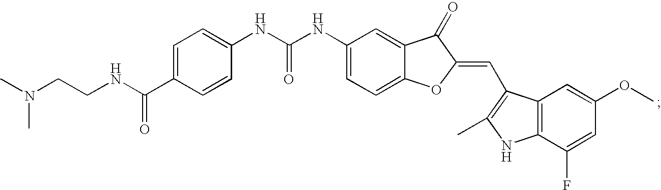

### (Z)-4-(3-(2-((2-Cyclopropyl-7-fluoro-5-methoxy-1H-indol-3-yl)methylene)-3-oxo-2,3-dihydrobenzofuran-5-yl)ureido)-N-(2-(dimethylamino)ethyl)-N-methylbenzamide

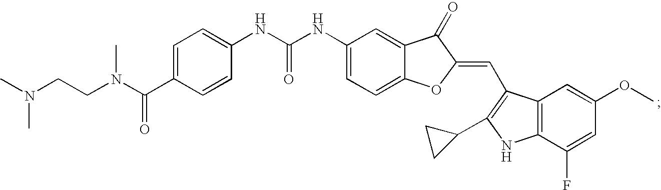

### (Z)-4-(3-(2-((2-Cyclopropyl-7-fluoro-5-methoxy-1H-indol-3-yl)methylene)-3-oxo-2,3-dihydrobenzofuran-5-yl)ureido)-N-(2-(dimethylamino)ethyl)benzamide

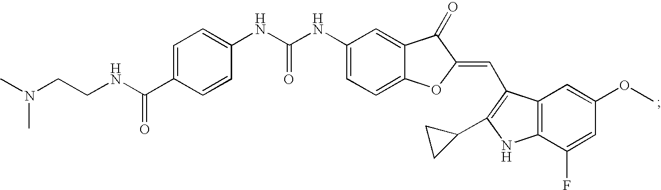

### (Z)-4-(3-(2-((2-Cyclopentyl-7-fluoro-5-methoxy-1H-indol-3-yl)methylene)-3-oxo-2,3-dihydrobenzofuran-5-yl)ureido)-N-(2-(dimethylamino)ethyl)-N-methylbenzamide

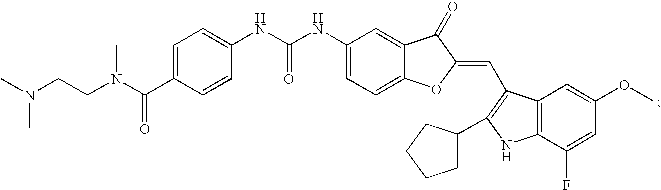

### (Z)-4-(3-(2-((2-Cyclopentyl-7-fluoro-5-methoxy-1H-indol-3-yl)methylene)-3-oxo-2,3-dihydrobenzofuran-5-yl)ureido)-N-(2-(dimethylamino)ethyl)benzamide

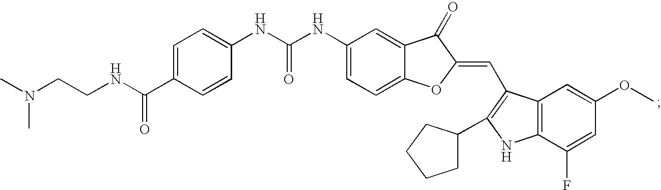

### Biological Evaluation

### PI3K-Alpha, PI3K-Beta, PI3K-Gamma, and PI3K-delta Fluorescence Polarization Assay Protocols

PI3-Kinase reactions were performed in 5 mM HEPES, pH 7, 2.5 mM MgCl₂, and 25 μM ATP, with diC8-PI(4,5)P2 (Echelon, Salt Lake City Utah) as substrate. Nunc 384-well black polypropylene fluorescent plates were used for PI3K assays. Reactions were quenched by the addition of EDTA to a final concentration of 10 mM. Final reaction volumes were 10 μl. For evaluation of PI3K inhibitors, 5 ng of enzyme (PI3K-alpha, beta, gamma, or delta) and 2.5 μM of substrate was used per 10 ml reaction volume, and inhibitor concentrations ranged from 100 μM to 20 μM; the final level of DMSO in reactions never exceeded 2%. Reactions were allowed to proceed for one hour at 25° C. After 1 hour, GST-tagged GRP1 (general receptor for phosphoinositides) PH domain fusion protein was added to a final concentration of 100 nM, and BODIPY-TMRI(1,3,4,5)P4 (Echelon) was also added to a final concentration of 5 nM. Final sample volumes were 25 μl with a final DMSO concentration of 0.8%. Assay Plates were read on Perkin-Elmer Envision plate readers with appropriate filters for Tamra [BODIPY-TMRI(1,3,4,5)P4]. Data obtained were used to calculate enzymatic activity and enzyme inhibition by inhibitor compounds.

### mTOR Enzyme Assay

The routine human TOR assays with purified enzyme are performed in 96-well plates by DELFIA format as follows. Enzyme is first diluted in kinase assay buffer (10 mM HEPES (pH 7.4), 50 mM NaCl, 50 mM β-glycerophosphate, 10 mM MnCl₂, 0.5 mM DTT, 0.25 μM microcystin LR, and 100 μg/mL BSA). To each well, 12 μL of the diluted enzyme is mixed briefly with 0.5 μL test inhibitor or the control vehicle dimethylsulfoxide (DMSO). The kinase reaction is initiated by adding 12.5 μL kinase assay buffer containing ATP and His6-S6K (substrate) to give a final reaction volume of 25 μL containing 800 ng/mL FLAG-TOR, 100 μM ATP and 1.25 μM His6-S6K. The reaction plate is incubated for 2 hours (linear at 1-6 h) at room temperature with gentle shaking and then terminated by adding 25 μL Stop buffer (20 mM HEPES, pH 7.4), 20 mM EDTA, 20 mM EGTA). The DELFIA detection of the phosphorylated His6-S6K (Thr-389) is performed at room temperature using a monoclonal anti-P(T389)-p70S6K antibody (1A5, Cell Signaling) labeled with Europium-N1-ITC (Eu) (10.4 Eu per antibody, PerkinElmer). The DELFIA Assay buffer and Enhancement solution are purchased from PerkinElmer. The terminated kinase reaction mixture (45 μL) is transferred to a MaxiSorp plate (Nunc) containing 55 μL PBS. The His6-S6K is allowed to attach for 2 hours after which the wells are aspirated and washed once with PBS. DELFIA Assay buffer (100 μL) with 40 ng/mL Eu—P(T389)-S6K antibody is added. The antibody binding is continued for 1 hour with gentle agitation. The wells are then aspirated and washed 4 times with PBS containing 0.05% Tween-20 (PBST). DELFIA Enhancement solution (100 μL) is added to each well and the plates are read in a PerkinElmer Victor model plate reader.

### In Vitro Cell Growth Assay

Cell lines used were human adenocarcinoma (LoVo), pancreatic (PC3), prostate (LNCap), breast (MDA468, MCF₇), colon (HCT116), renal (HTB44 A498), and ovarian (OVCAR3) tumor cell lines. The tumor cells were plated in 96-well culture plates at approximately 3000 cells per well. One day following plating, various concentrations of inhibitors in DMSO were added to cells (final DMSO concentration in cell assays was 0.25%). Three days after drug treatment, viable cell densities were determined by cell mediated metabolic conversion of the dye MTS, a well-established indicator of cell proliferation in vitro. Cell growth assays were performed using kits purchased from Promega Corporation (Madison, Wis.), following the protocol provided by the vendor. Measuring absorbance at 490 nm generated MTS assay results. Compound effect on cell proliferation was assessed relative to untreated control cell growth. The drug concentration that conferred 50% inhibition of growth was determined as IC₅₀ (μM). IC₅₀ values of about 2 nM to several μM were observed in the various tumor lines for compounds of this invention.

TABLE VIII

| | | | | PI3Kδ | mTOR |
|---|---|---|---|---|---|
| | PI3Kα | PI3Kγ | PI3Kβ | Avg. | Kinase |
| Com- | Avg. | Avg. | Avg. | IC₅₀ | Avg. IC₅₀ |
| pound | IC₅₀ (nM) | IC₅₀ (nM) | IC₅₀ (nM) | (nM | (μM) |
| 1 | >10000.0 | | | | |
| 2 | >10000.0 | | | | >20.00 |
| 3 | >10000.0 | | | | >20.00 |
| 4 | 12262.50 | | | | >20.00 |
| 5 | >10000.0 | | | | >20.00 |
| 6 | 2396.00 | | | | 2.85 |
| 7 | >10000.0 | | | | |
| 8 | >10000.0 | | | | >20.00 |
| 9 | >10000.0 | | | | |
| 10 | >10000.0 | | | | |
| 11 | >10000.0 | | | | |
| 12 | >10000.0 | | | | |
| 13 | 6754.00 | | | | 9.2 |
| 14 | 1033.50 | 2781 | 736 | 381 | 0.13 |
| 15 | 2664.50 | | | | 0.09 |
| 16 | 1105.70 | 6949 | | | 0.8 |
| 17 | 2491.50 | | | | |
| 18 | 2550.00 | | | | |
| 19 | 1982.50 | | | | |
| 20 | >10000.0 | | | | |
| 21 | >10000.0 | | | | |
| 22 | >10000.0 | | | | |
| 23 | >10000.0 | | | | |
| 24 | >10000.0 | | | | |
| 25 | >10000.0 | | | | |
| 26 | 3806.50 | | | | |
| 27 | 5473.50 | | | | |
| 28 | 2426.00 | | | | |
| 29 | >10000.0 | | | | |
| 30 | 12824.50 | | | | |
| 31 | 9597.50 | | | | |
| 32 | 6475.00 | | | | |
| 33 | >10000.0 | | | | |
| 34 | >10000.0 | | | | >20.00 |
| 35 | 1943.00 | | | | |
| 36 | >10000.0 | | | | |
| 37 | 2348.50 | | | | |
| 38 | >10000.0 | | | | |
| 39 | >10000.0 | | | | |
| 40 | >10000.0 | | | | |
| 41 | >10000.0 | | | | |
| 42 | >10000.0 | | | | |
| 43 | >10000.0 | | | | |
| 44 | 541.70 | 1458 | | | 0.19 |
| 45 | 799.30 | 1820 | | | 0.28 |
| 46 | 1580.50 | | | | 0.1225 |
| 47 | 3196.50 | | | | 0.5 |
| 48 | 3415.00 | | | | >5.00 |
| 49 | 2432.50 | | | | >5.00 |
| 50 | 422.00 | 1624.5 | 358 | 387.3 | 3.7 |
| 51 | >10000.0 | | | | >5.00 |
| 52 | 1888.50 | | | | >5.00 |
| 53 | 182.00 | 1469.7 | 132 | 70.3 | >30 |
| 54 | 110.80 | 1060.3 | 98 | 33.5 | 9.6 |
| 55 | 72.70 | 715.7 | 72 | 32 | 10 |
| 56 | 190.20 | 1954.3 | 251.5 | 107.5 | >20.00 |
| 57 | 4500.00 | | | | |
| 58 | 310.00 | 3534.5 | | | 15 |
| 59 | 30.20 | 269 | 173.5 | 70 | 0.265 |
| 60 | 491.70 | 3628 | | | 11 |
| 61 | 179.00 | 2220.5 | 566.5 | 374 | 0.19 |
| 62 | 673.30 | 3768 | | | 16 |
| 63 | 431.30 | 5000 | | | >20.00 |
| 64 | 1715.00 | | | | |
| 65 | 383.70 | 3773 | | | >20.00 |
| 66 | 232.30 | 2910 | | | |
| 67 | 327.70 | 302 | | | 8.4 |
| 68 | 481.00 | 486 | | | |
| 69 | 399.00 | 3500 | | | >20.00 |
| 70 | 3.20 | 13.4 | 26 | 5.3 | <0.03 |
| 71 | 74.20 | 439.5 | | | |
| 72 | 3649.50 | | | | 0.02125 |
| 73 | 2.70 | 25 | 8 | 5.8 | 0.0027 |
| 74 | 12000.00 | | | | 9.8 |
| 75 | 2520.00 | | | | 5.9 |
| 76 | >10000.0 | | | | 10 |
| 77 | >10000.0 | | | | 17.5 |
| 78 | 516.30 | 1200 | | | 2.9 |
| 79 | 1649.50 | | | | |
| 80 | >10000.0 | | | | 0.49 |
| 81 | 234.00 | 5000 | | | >20.00 |
| 84 | 105.80 | 586.3 | 546.7 | 110.3 | 0.06525 |
| 85 | 1146.00 | | | | |
| 86 | 184.20 | 355 | 98 | 47 | 0.08475 |
| 87 | 13.30 | 142 | 72 | 6 | <0.024 |
| 88 | 9.70 | 73.7 | 52 | 3.5 | <0.021 |
| 89 | 6.90 | 54.3 | 19 | 8 | 0.0035 |
| 90 | 201.50 | 1223 | | | 1.595 |
| 91 | <2.2 | 9.5 | 5.5 | 1.8 | <0.05 |
| 92 | 2044.00 | | | | 2.6 |
| 93 | 2381.00 | | | | >5.00 |
| 94 | 2236.00 | 5862 | 671 | 1016 | >5.00 |
| 95 | 14.70 | 402 | 27 | 2 | 0.15333 |
| 96 | 11.30 | 238 | 10 | 2 | <0.031 |
| 97 | 8.70 | 138.5 | 9 | 0.9 | <0.047 |
| 98 | 9.40 | 248 | 10.5 | 1 | 0.0585 |
| 99 | 8.30 | 331 | 8 | 2 | 0.0725 |
| 100 | 12.00 | 283.5 | 12.5 | 3 | 0.0555 |
| 101 | 5.30 | 376.7 | 6.5 | 1 | 0.046 |
| 102 | 8.50 | 75.5 | 45 | 1.9 | <0.025 |
| 103 | 6.60 | 108.5 | 22 | 2 | 0.108 |
| 104 | 5.70 | 162 | 19 | 2.3 | 0.13 |
| 105 | 9.60 | 724.3 | 50.5 | 2 | 0.14 |
| 106 | 5.30 | 403.7 | 17.5 | 2 | 0.1175 |
| 107 | 11.30 | 154.5 | 112 | 5.8 | <0.025 |
| 108 | 5.00 | 473.5 | 7.5 | <2.0 | <0.034 |
| 109 | 4.30 | 321.5 | 18 | 2.8 | <0.023 |
| 110 | 9.30 | 282.5 | 32 | 6.5 | 0.0175 |
| 111 | 36.30 | 975.5 | 169 | 21.5 | 0.215 |
| 112 | 42.00 | 646 | 180.5 | 6 | 0.43 |
| 113 | 37.00 | 1358 | 243 | 7 | 1.65 |
| 114 | 40.00 | 2419.5 | 221.5 | 11.5 | 2.125 |
| 115 | 39.30 | 1188.5 | 136 | 8.5 | 0.98 |
| 116 | 37.00 | 1342.5 | 118.5 | 5 | 1.425 |
| 117 | 1842.00 | 8604 | | | |
| 118 | 288.00 | 932 | | | |
| 119 | 3833.00 | >10000.0 | | | |
| 120 | 1539.00 | 10330 | | | |
| 121 | 316.50 | 5430 | | | |
| 122 | 137.00 | 1502.5 | | | |
| 123 | 1220.00 | >10000.0 | | | |
| 124 | 64.50 | 1219 | | | |
| 125 | 380.50 | 933 | | | |
| 129 | 488.00 | 4498 | | | |
| 130 | 650.50 | 4135 | | | |
| 131 | 478.00 | 3971 | | | |
| 132 | 337.00 | 4153 | | | |
| 133 | 230.50 | 954 | | | |
| 134 | 204.00 | 3169 | | | |
| 135 | 1302.00 | 4768 | | | |
| 136 | 235.50 | 2483 | | | |
| 137 | 478.00 | 2768 | | | |
| 143 | 1854.50 | 6655 | | | 4.45 |
| 144 | 1598.00 | | | | |
| 145 | 825.00 | | | | |
| 146 | 850.00 | | | | |
| 147 | 2454.50 | 6000 | | | 1.085 |
| 148 | 2676.50 | 4709 | | | 0.5 |
| 149 | 1585.50 | 2011 | | | 6.15 |
| 150 | 2230.00 | 2188 | | | 6.45 |
| 151 | 2112.50 | 4220 | | | 0.71 |
| 152 | 4183.00 | 4469 | | | 1.45 |
| 153 | 1581.00 | 11000 | | | 2.9 |
| 154 | 1584.50 | 11000 | | | 0.54 |
| 155 | 516.00 | 5354 | | | 0.08 |
| 156 | 1291.00 | 6000 | | | 1.6 |
| 157 | 1515.00 | 2427 | | | 2.5 |
| 158 | 2281.00 | 4879 | | | 4.6 |
| 159 | 2858.00 | 5125 | | | 1.8 |
| 160 | 1014.00 | | | | 0.82 |
| 161 | 1761.00 | | | | 0.6 |
| 162 | 2454.00 | | | | >20.00 |
| 164 | 250.50 | 3846.5 | | | 5.2 |
| 165 | 9500.00 | >10000.0 | | | >20.00 |
| 166 | 28.00 | 179.5 | 46 | 59 | 1.7 |
| 167 | 6756.00 | | | | 5.3 |
| 168 | 6539.00 | | | | >20.00 |
| 169 | 3595.00 | | | | 9.1 |
| 170 | 5773.00 | | | | >20.00 |
| 171 | 2035.00 | >10000.0 | | | >20.00 |
| 172 | 936.50 | 10000 | | | 19 |
| 173 | 766.00 | >10000.0 | | | 17 |
| 174 | 1509.50 | >10000.0 | | | 7.1 |
| 175 | 1037.50 | >10000.0 | | | 9 |
| 176 | 1550.00 | | | | 8.7 |
| 177 | 187.00 | 3363 | | | 9.1 |
| 178 | 1474.00 | | | | 12.5 |
| 179 | 2470.00 | | | | 16 |
| 180 | 3115.00 | | | | >20.00 |
| 181 | 502.00 | 1752 | | | 20 |
| 182 | 1112.50 | >10000.0 | | | 12 |
| 183 | 1195.00 | | | | 6.1 |
| 184 | 101.70 | 543.5 | 155 | 193 | 0.19 |
| 185 | 32.30 | 403 | 96.5 | 38 | 0.13 |
| 186 | 5764.00 | | | | 16 |
| 187 | 4995.00 | | | | >20.00 |
| 188 | 1796.50 | >10000.0 | | | 0.26 |
| 189 | 2714.00 | 11047 | | | 0.14 |
| 190 | 2121.50 | 8536 | | | 0.12 |
| 191 | 4179.00 | | | | 0.39 |
| 192 | 2423.00 | | | | 0.39 |
| 193 | 1117.50 | 7658 | | | 0.085 |
| 194 | 837.00 | 3286 | | | 0.23 |
| 195 | 1034.00 | | | | 0.38 |
| 196 | 1690.00 | | | | 0.39 |
| 197 | 1096.00 | 9162 | | | 0.28 |
| 198 | 4870.00 | | | | 4.5 |
| 199 | 1558.00 | | | | 1.4 |
| 200 | 1093.00 | 3073 | | | 0.22 |
| 201 | 712.50 | 5456 | | | 0.12 |
| 202 | 1045.50 | 7000 | | | 0.095 |
| 203 | 1408.50 | 6619 | | | 0.15 |
| 204 | 1155.50 | 6433 | | | 0.19 |
| 205 | 479.00 | 2374 | | | 0.11 |
| 206 | 573.50 | 1048 | | | 0.065 |
| 207 | 1294.00 | | | | 0.3 |
| 208 | 954.00 | | | | 0.3 |
| 209 | 1155.00 | | | | 0.49 |
| 210 | 673.50 | 1476 | | | 0.19 |
| 211 | 1065.00 | | | | 0.59 |
| 212 | 1056.00 | | | | 0.64 |
| 213 | 770.00 | 2649 | | | 0.17 |
| 214 | 4909.00 | | | | 2.55 |
| 215 | 264.50 | 2606 | | | 0.29 |
| 216 | 9500.00 | | | | 11.5 |
| 217 | 1420.00 | | | | 2.4 |
| 218 | 796.00 | | | | 1.65 |
| 219 | 1004.00 | | | | >20.00 |
| 220 | 1983.00 | | | | 4.3 |
| 221 | 1159.00 | | | | >20.00 |
| 222 | 4457.00 | | | | 2.1 |
| 223 | 3662.00 | >10000.0 | | | 0.06 |
| 224 | 751.00 | >10000.0 | | | 0.48 |
| 225 | 968.00 | >10000.0 | | | 0.7 |
| 226 | 1647.00 | >10000.0 | | | 0.67 |
| 227 | 756.50 | 7515 | | | 3.8 |
| 228 | 3570.50 | >10000.0 | | | 0.98 |
| 229 | 735.00 | >10000.0 | | | 8.3 |
| 230 | 2021.50 | >10000.0 | | | 3.4 |
| 231 | 3501.50 | >10000.0 | | | 20 |
| 232 | 610.50 | 6000 | | | >20.00 |
| 233 | 2042.00 | | | | 0.85 |
| 234 | >10000.0 | | | | 14 |
| 235 | 4030.00 | | | | 4 |
| 236 | 1466.00 | | | | 1.6 |
| 237 | 2475.00 | | | | >20.00 |
| 238 | 288.00 | 2818 | | | 0.36 |
| 239 | 4939.00 | | | | 5.9 |
| 240 | 3331.00 | | | | 2.7 |
| 241 | 4706.00 | | | | >20.00 |
| 242 | 999.50 | >10000.0 | | | >20.00 |
| 243 | 1048.00 | | | | 0.48 |
| 244 | 1965.00 | | | | 18 |
| 245 | 1489.00 | | | | >20.00 |
| 246 | 1026.50 | >10000.0 | | | 17 |
| 247 | 3152.00 | | | | 2.9 |
| 248 | 1037.00 | >10000.0 | | | >20.00 |
| 249 | 4814.00 | | | | >20.00 |
| 250 | 3555.00 | | | | >20.00 |
| 251 | 2386.00 | | | | >20.00 |
| 252 | 555.50 | 10000 | | | 1.5 |
| 253 | 1426.00 | | | | 3.3 |
| 254 | 1663.00 | | | | 0.65 |
| 255 | 2464.00 | | | | 0.3 |
| 256 | >10000.0 | | | | 0.28 |
| 257 | 179.70 | 2239 | | | 12 |
| 259 | | | | | |
| 260 | 1674.00 | | | | 4.5 |
| 261 | 1977.00 | | | | 0.68 |
| 262 | >10000.0 | | | | 0.42 |
| 263 | 1683.00 | | | | 0.26 |
| 264 | 3932.00 | | | | 1.7 |
| 265 | >10000.0 | | | | 1.3 |
| 266 | 290.00 | | | | 0.81 |
| 267 | 2059.00 | | | | 0.086 |
| 268 | >5739.0 | 2691.5 | | | 0.0295 |
| 269 | 1639.50 | 2212 | | | 1.06 |
| 270 | 232.00 | 1084 | | | |
| 271 | | | | | |
| 272 | 1318.00 | | | | |
| 273 | 9618.00 | | | | |
| 274 | 287.20 | 255 | | | |
| 275 | 2801.00 | | | | |
| 276 | >10000.0 | | | | |
| 277 | 268.20 | 264 | | | |
| 278 | 3102.00 | | | | |
| 279 | >10000.0 | | | | |
| 280 | 4558.00 | | | | |
| 281 | >10000.0 | | | | |
| 282 | 1419.00 | | | | |
| 283 | >10000.0 | | | | |
| 284 | >10000.0 | | | | |
| 285 | >10000.0 | | | | >20.00 |
| 286 | >10000.0 | | | | |
| 287 | >10000.0 | | | | |
| 288 | >10000.0 | | | | |
| 289 | 10715.50 | | | | |
| 290 | 9172.50 | | | | |
| 291 | >10000.0 | | | | |
| 292 | >10000.0 | | | | |
| 293 | >10000.0 | | | | |
| 294 | >10000.0 | | | | |
| 295 | >10000.0 | | | | |
| 296 | 12000.00 | | | | |
| 297 | >10000.0 | | | | |
| 298 | 9185.50 | | | | |
| 299 | >10000.0 | | | | |
| 300 | 2860.00 | 9500 | | | 0.1325 |
| 301 | 1231.00 | 2307 | | | 0.125 |
| 302 | 17.00 | 764 | 14 | <2.5 | 0.3 |
| 303 | 2.20 | 70 | 3.5 | <2.0 | 0.00193 |
| 304 | 54.00 | 65 | 127.5 | 17 | 0.062 |
| 305 | 5.00 | 228 | 28.5 | 5 | 0.205 |
| 306 | 8.50 | 143.5 | 20.5 | 3 | 0.13 |
| 307 | 18.50 | 1816.5 | 72.5 | 2.5 | 3.7 |
| 308 | 25.50 | 1026.5 | 23.5 | 5.5 | 0.27 |
| 309 | 2.00 | 268.5 | 6.5 | 2 | <0.027 |
| 310 | <2.1 | 13.5 | 11.5 | 0.9 | 0.092 |
| 311 | 8.00 | 143.5 | | | 0.0885 |
| 312 | 9.50 | 81.5 | | | 0.1525 |
| 313 | 3.00 | 16.5 | | | 0.00053 |
| 314 | 29.00 | 116.5 | | | 0.155 |
| 315 | 3.00 | 26.5 | | | 0.0042 |
| 316 | <2.7 | 278 | 25 | 1.5 | 0.0145 |
| 317 | 17.00 | 92.5 | 64 | 8.5 | 0.0775 |
| 318 | 83.30 | 686.5 | 739.5 | 27 | 0.25 |
| 319 | 38.30 | 117 | 108.5 | 3.5 | 0.265 |
| 320 | 3.00 | 36.5 | 22 | 0.8 | 0.01275 |
| 321 | 2.00 | 15.5 | 10 | 1.8 | 0.00685 |
| 322 | 39.80 | 178 | | | 0.023 |
| 323 | 20.50 | 605 | 119.5 | 4 | 0.93 |
| 324 | 91.50 | 655.5 | | | 0.365 |
| 325 | 281.50 | 298 | | | 0.084 |
| 326 | 58.00 | 150 | 88 | 9.5 | 0.54 |
| 327 | 11.00 | 39 | 9.5 | 2.5 | <0.030 |
| 328 | 6.00 | 15.5 | 2.5 | 2 | <0.024 |
| 329 | 12.50 | 130 | 51.6 | 18.5 | 0.039 |
| 330 | <1.8 | 3 | 1 | 1.5 | <0.017 |
| 331 | 4.00 | 39.5 | 5.5 | 6.5 | 0.0495 |
| 332 | 5.00 | 22 | 13.3 | 7.8 | <0.021 |
| 333 | 66.00 | 158.5 | 202.5 | 51 | 0.053 |
| 334 | 1.30 | 2.2 | 1.2 | 1.5 | <0.017 |
| 335 | 9.00 | 588.5 | 22 | 2 | 1.65 |
| 336 | 4.00 | 61.7 | 4.1 | 1 | <0.022 |
| 337 | 13.00 | 7 | 75.3 | 9.7 | 0.0205 |
| 338 | 22.00 | 666 | 57.7 | 3 | >0.16 |
| 339 | 5.50 | 8.5 | 93.7 | 20.3 | 0.0395 |
| 340 | 2.00 | 3 | 7.5 | 1.6 | <0.00082 |
| 341 | 1.70 | 22.8 | 7.1 | 0.8 | 0.01075 |
| 342 | 4.30 | 32.5 | 16.3 | 1.5 | 0.0067 |
| 343 | 12.00 | 292.5 | 71 | 2.5 | >0.16 |
| 344 | 4.40 | 99 | 5.9 | 0.9 | 0.00525 |
| 345 | 24.00 | 692 | 100.3 | 2 | >4.00 |
| 346 | 6.10 | 55 | 7.7 | 3 | 0.077 |
| 347 | 407.50 | 8669.5 | | | >4.00 |
| 348 | 11.50 | 60.5 | 76.5 | 12.5 | 0.0175 |
| 349 | 82.50 | 5589.5 | 931 | 19 | >20.00 |
| 350 | 114.50 | 6874.5 | | | >20.00 |
| 351 | 66.00 | 3272.5 | 505 | 9 | >0.80 |
| 352 | 115.50 | 3927 | | | >0.80 |
| 353 | 4.00 | 38 | 8 | 2 | 0.022 |
| 354 | 6.50 | 55 | 44 | 14.5 | 0.018 |
| 355 | 8.60 | 36 | 17 | 2.5 | 0.013 |
| 356 | 15.50 | 408.5 | 116 | 1.2 | >0.80 |
| 357 | 11.00 | 322 | 98.5 | <1.0 | >0.80 |
| 358 | 7.50 | 239.5 | 95.5 | <1.0 | >0.80 |
| 359 | 11.00 | 232.5 | 18.5 | 1.5 | >0.80 |
| 360 | 1.50 | 3.2 | 1.3 | 0.3 | 0.00125 |
| 361 | 306.00 | 3436.5 | | | >0.80 |
| 362 | 10.50 | 329 | 8 | 1.5 | 0.21 |
| 363 | 1648.00 | 1076 | | | 3.5 |
| 364 | 698.00 | 1285 | | | 0.1225 |
| 365 | 437.50 | 1964.5 | | | 0.104 |
| 366 | 1089.50 | 3929 | | | 0.0755 |
| 367 | 1436.00 | 5422 | | | 0.5 |
| 368 | 1000.00 | 4565 | | | 0.0445 |
| 369 | 1048.00 | 5000 | | | 0.0845 |
| 370 | 1074.00 | 3000 | | | 0.014 |
| 371 | 1010.00 | 763 | | | 0.73 |
| 372 | 738.00 | 3000 | | | 0.026 |
| 373 | 4772.00 | >10000.0 | | | 0.3 |
| 374 | 2613.00 | 4197 | | | 0.11 |
| 375 | 2812.00 | 9500 | | | 0.028 |
| 376 | 2076.00 | 3796.5 | | | 0.1275 |
| 377 | 496.00 | 4071 | | | 0.007 |
| 378 | 971.50 | 3399.5 | | | 0.107 |
| 379 | 561.50 | 1090.5 | | | 0.029 |
| 380 | 431.50 | 3256 | | | 0.089 |
| 381 | 2735.00 | 3954.5 | | | 0.18 |
| 382 | 2918.50 | 4680.5 | | | 0.535 |
| 383 | 750.50 | 2248.5 | | | 0.01225 |
| 384 | >10000.0 | 2447 | | | >20.00 |
| 385 | 2794.00 | 7630 | | | 0.018 |
| 386 | 1923.00 | 5779 | | | 0.0695 |
| 387 | 3519.00 | 4591 | | | 0.34 |
| 388 | 1347.00 | 3275 | | | 0.2 |
| 389 | 1869.00 | 9858 | | | 0.0115 |
| 390 | 1124.00 | 2043 | | | 0.09 |
| 391 | 1151.00 | 2394 | | | 0.0385 |
| 392 | 453.00 | >7179.0 | | | 0.017 |
| 393 | 608.00 | 2518.5 | | | 0.0235 |
| 394 | 459.50 | 4122.5 | | | 0.069 |
| 395 | 581.50 | 3885 | | | 0.095 |
| 396 | 547.00 | 447.5 | | | 0.03 |
| 397 | 1840.00 | 8529 | | | 0.02 |
| 398 | 2756.00 | 5260 | | | 0.111 |
| 399 | 805.50 | >10000.0 | | | 0.0695 |
| 400 | >10000.0 | >10000.0 | | | >19.5 |
| 401 | 267.00 | 2684.5 | | | 0.0615 |
| 402 | 3742.00 | 5937 | | | 0.355 |
| 403 | 1640.00 | 7708 | | | 0.1225 |
| 404 | 935.00 | 1648 | | | 0.061 |
| 405 | >10000.0 | >10000.0 | | | >20.00 |
| 406 | 10000.00 | >10000.0 | | | >20.00 |
| 407 | >10000.0 | >10000.0 | | | >20.00 |
| 408 | 3306.00 | 1943 | | | >20.00 |
| 409 | >10000.0 | 1993 | | | >20.00 |
| 410 | 3.00 | 116.5 | | | 0.00064 |
| 411 | 11.50 | 984.5 | | | 0.047 |
| 412 | 10.50 | 887.5 | | | 2.8 |
| 413 | 7819.00 | 5612 | | | >20.00 |
| 414 | 55.00 | 281.5 | | | 1.85 |
| 415 | 62.00 | 465.5 | | | 1.575 |
| 416 | 1127.00 | 2662 | | | >20.00 |
| 417 | 4736.00 | 2103 | | | >20.00 |
| 418 | 1385.00 | 9500 | | | 10.3 |
| 419 | 1070.00 | >10000.0 | | | 0.024 |
| 420 | 708.00 | >10000.0 | | | 0.0315 |
| 421 | >10000.0 | >10000.0 | | | >20.00 |
| 422 | 9000.00 | 7639 | | | >20.00 |
| 423 | >10000.0 | >10000.0 | | | >20.00 |
| 424 | 4024.00 | >10000.0 | | | >20.00 |
| 425 | 5042.00 | 8655 | | | >20.00 |
| 426 | 6249.00 | >10000.0 | | | >20.00 |
| 427 | 9529.00 | 3027 | | | >20.00 |
| 428 | >5000.0 | >5000.0 | | | >20.00 |
| 429 | 67.50 | 3135 | | | 0.5 |
| 430 | 7.50 | 197 | | | 0.115 |
| 432 | >10000.0 | 8139 | | | 2.025 |
| 433 | >10000.0 | >10000.0 | | | >20.00 |
| 434 | >10000.0 | >10000.0 | | | >20.00 |
| 435 | >10000.0 | >10000.0 | | | >20.00 |
| 436 | >10000.0 | >10000.0 | | | >20.00 |
| 437 | 1602.00 | >10000.0 | | | <0.023 |
| 438 | 2984.00 | 9000 | | | >20.00 |
| 439 | 4281.00 | >10000.0 | | | 0.03375 |
| 441 | >10000.0 | >10000.0 | | | >20.00 |
| 442 | >5100.0 | >5100.0 | | | >20.00 |
| 443 | >5100.0 | >5100.0 | | | 1.235 |
| 444 | 65.50 | 100 | | | 0.01235 |
| 446 | 3.50 | 81 | | | 0.00375 |
| 447 | 1.70 | 104.5 | | | 0.0013 |
| 448 | 142.00 | 5126.5 | | | >20.00 |
| 449 | 165.50 | >11000.0 | | | >20.00 |
| 450 | 662.50 | 5000 | | | >20.00 |
| 451 | 375.00 | 7738 | | | >20.00 |
| 452 | 49.50 | 989.5 | | | 1.95 |
| 453 | 49.50 | 550 | | | 2.2 |
| 454 | 32.00 | 516.5 | | | 1.3 |
| 455 | 39.00 | 612 | | | 1.4 |
| 456 | 90.50 | 4599.5 | | | >20.00 |
| 457 | 74.00 | 5054.5 | | | >20.00 |
| 458 | 134.50 | 4354.5 | | | >20.00 |
| 459 | 190.50 | 6694 | | | >20.00 |
| 460 | 18.00 | 1380 | | | 6.5 |
| 461 | 19.50 | 1065 | | | 12.5 |
| 462 | 21.50 | 1048.5 | | | 0.635 |
| 463 | 51.50 | 971 | | | 2 |
| 464 | 13.50 | 1446 | | | 0.0375 |
| 465 | 29.50 | 475.5 | | | >20.00 |
| 466 | 73.00 | 1275 | | | 7.5 |
| 467 | 50.50 | 1788 | | | 0.425 |
| 468 | 48.50 | 3199.5 | | | 2.2 |
| 469 | 23.50 | 941.5 | | | 0.165 |
| 470 | 35.50 | 655.5 | | | 2.3 |
| 471 | 62.50 | 139 | | | >4000 |
| 472 | 163.50 | 1130 | | | 2750 |
| 473 | 162.00 | 2611 | | | >4000 |
| 474 | 246.00 | 1391.5 | | | >4000 |
| 475 | >10000 | >10000 | | | >4000 |
| 476 | 412.00 | 1644 | | | 155 |
| 477 | 1121.00 | 11000 | | | 155 |
| 478 | 1198.00 | 1744 | | | 46.5 |
| 479 | 297.50 | >10000 | | | >4000 |
| 480 | 81.50 | 3514.5 | | | >4000 |
| 481 | >10000 | >10000 | | | >4000 |
| 482 | >10000 | >10000 | | | >4000 |
| 483 | 9859.00 | 5224 | | | >4000 |
| 484 | 1659.00 | >10000 | | | >4000 |
| 485 | >10000 | >10000 | | | >4000 |
| 486 | 1455.00 | 1609 | | | >4000 |
| 487 | 10204.00 | >10000 | | | >4000 |
| 488 | >10000 | >10000 | | | >4000 |
| 489 | 6217.00 | >10000 | | | 3800 |
| 490 | 674.00 | >10000 | | | >4000 |
| 491 | 465.00 | 1314 | | | >4000 |
| 492 | 656.00 | >10000 | | | >4000 |
| 493 | 3.00 | 15.5 | | | 1.05 |
| 494 | >10000 | 9349 | | | >4000 |
| 495 | >10000 | >10000 | | | >4000 |
| 496 | 1044.00 | 4771 | | | 13.5 |
| 497 | 963.00 | 1210 | | | 125 |
| 498 | 63.50 | 392 | | | 690 |
| 499 | 342.00 | 3556 | | | 325 |
| 500 | 589.00 | 5000 | | | >4000 |
| 501 | 701.00 | 7000 | | | >4000 |
| 502 | 592.00 | 4159 | | | >4000 |
| 503 | 106.50 | 811 | | | 590 |
| 504 | 122.00 | 541 | | | >4000 |
| 505 | 120.50 | 813 | | | >4000 |
| 506 | 133.00 | 1511.5 | | | 3050 |
| 507 | 78.50 | 1538 | | | 3600 |
| 508 | 8504.00 | >10000 | | | >4000 |
| 509 | 484.00 | 2466 | | | >4000 |
| 510 | 8.00 | 56 | | | 110 |
| 511 | 21.00 | 154.5 | | | 1005 |
| 512 | 36.50 | 1400.5 | | | >4000 |
| 513 | 1536.00 | >10000 | | | >4000 |
| 514 | 1376.00 | 4102 | | | >4000 |
| 515 | 1174.00 | 2512 | | | >4000 |
| 516 | 9746.00 | 5000 | | | >4000 |
| 517 | 82.50 | 714 | | | >4000 |
| 518 | 451.00 | 1149 | | | >4000 |
| 519 | 272.50 | 6051 | | | 1050 |
| 520 | 72.50 | 4020 | | | 1925 |
| 521 | 2466.00 | 8511 | | | 1450 |
| 522 | >10000 | >10000 | | | >4000 |
| 523 | >10000 | >10000 | | | >4000 |
| 524 | 12000.00 | >10000 | | | >4000 |
| 525 | 1275.00 | >10000 | | | >4000 |
| 526 | 6188.50 | >10000 | | | >4000 |
| 527 | 2991.00 | 3815 | | | >4000 |
| 528 | >10000 | >10000 | | | >4000 |
| 529 | 221.50 | 8046.5 | | | >4000 |
| 530 | >10000 | >10000 | | | 550 |
| 531 | 340.00 | 2527 | | | 59 |
| 532 | >10000 | >10000 | | | >4000 |
| 533 | 2.20 | 388.5 | | | 8.5 |
| 534 | 5880.00 | 476 | | | 1165 |
| 535 | >10000 | >10000 | | | 4000 |
| 536 | 58.50 | 545.5 | | | 150 |
| 537 | 421.00 | >10000 | | | >4000 |
| 538 | 820.00 | >10000 | | | >4000 |
| 539 | >10000 | >10000 | | | >4000 |
| 540 | 103.50 | 1546 | | | >4000 |
| 541 | 1381.00 | 9099 | | | >4000 |
| 542 | 359.00 | 5000 | | | 715 |
| 543 | >10000 | >10000 | | | >4000 |
| 544 | 266.00 | 2149 | | | 190 |
| 545 | 40.50 | 1242.67 | | | >4000 |
| 546 | 630.00 | 7358.5 | | | >4000 |
| 547 | 63.00 | 2115.3 | | | 1250 |
| 548 | 64.50 | 2791.3 | | | >4000 |
| 549 | 13.50 | 2976.5 | | | >4000 |
| 550 | 64.50 | 1130 | | | 2300 |
| 551 | 191.50 | 3146.5 | | | 2800 |
| 552 | 355.50 | >10000 | | | >4000 |
| 553 | 31.00 | 4828.5 | | | 640 |
| 554 | 73.00 | 5264 | | | 2750 |
| 555 | 48.00 | 1886.5 | | | >4000 |
| 556 | 2.15 | 835 | | | 37.5 |
| 557 | 1195.00 | >10000 | | | 205 |
| 558 | >10000 | 2165 | | | >4000 |
| 559 | 2.25 | 537 | | | 12.5 |
| 560 | 132.50 | 1601.5 | | | 775 |
| 561 | 3.15 | 377.5 | | | 4.8 |
| 562 | 2.85 | 209.5 | | | 7.65 |
| 563 | 35.50 | 5218 | | | >4000 |
| 564 | 95.50 | 7620 | | | >4000 |
| 565 | 114.50 | >10000 | | | >4000 |
| 566 | 93.50 | 7791.5 | | | 2100 |
| 567 | 164.00 | 1548 | | | >4000 |
| 568 | 477.00 | 3391 | | | >4000 |
| 569 | 242.00 | 6495 | | | >4000 |
| 570 | 8409.00 | 6494 | | | >4000 |
| 571 | 612.50 | 6442 | | | >4000 |
| 572 | 1655.00 | 2320 | | | >4000 |
| 573 | 52.00 | 1587.5 | | | >4000 |
| 574 | 97.50 | 2993 | | | 1250 |
| 575 | 1.25 | 205.5 | | | 16 |
| 576 | 0.60 | 27.5 | | | 60 |
| 577 | 1.10 | 145.5 | | | 9.95 |
| 578 | 38.00 | 2184.5 | | | >4000 |
| 579 | 30.00 | 128.5 | | | 150 |
| 580 | 53.00 | 1355.5 | | | 1400 |
| 581 | 2.00 | 26.5 | | | 3.25 |
| 582 | 183.00 | 3574 | | | >4000 |
| 583 | 222.50 | 6487 | | | >4000 |
| 584 | 1.10 | 26 | | | 33.5 |
| 585 | 11.00 | 138 | | | 93 |
| 586 | 3.50 | 1013 | | | 190 |
| 587 | 4.00 | 982 | | | 125 |
| 588 | 3.50 | 830 | | | 290 |
| 589 | 4.50 | 966.5 | | | 125 |
| 590 | 3.67 | 1018 | | | 36 |
| 591 | 8.00 | 9500 | | | 80.5 |
| 592 | 5.50 | 8900 | | | 19.5 |
| 593 | 12.50 | 775.5 | | | 21.5 |
| 594 | 73.50 | 2203 | | | >4000 |
| 595 | 222.50 | 1533 | | | >4000 |
| 596 | 1.70 | 279 | | | 49.5 |
| 597 | 3.50 | 447.67 | | | 123.5 |
| 598 | 30.75 | 416.67 | | | 27.5 |
| 599 | 10.00 | 949.6666667 | | | 72.5 |
| 600 | 24.00 | 128.5 | | | 22 |
| 601 | 8.00 | 77 | | | 49.5 |
| 602 | 40.50 | 316 | | | 320 |
| 603 | 6.50 | 102.5 | | | 43 |
| 604 | 10.50 | >10000 | | | 15.5 |
| 605 | 19.00 | 315.5 | | | 350 |
| 606 | 8.00 | 113 | | | 78 |
| 607 | 4.50 | 4219.5 | | | 134.5 |
| 608 | 15.50 | 135.5 | | | 38 |
| 609 | 15.00 | 445.5 | | | 240 |
| 610 | 11.50 | 106 | | | 58 |
| 611 | 19.50 | 712.5 | | | 290 |
| 612 | 15.50 | 121.5 | | | 42 |
| 613 | 12.50 | 153.5 | | | 120 |
| 614 | 3.50 | 355.5 | | | 225 |
| 615 | 9.50 | 136.5 | | | 165 |
| 616 | 0.40 | 4.8 | | | 0.013 |
| 617 | 1.90 | 246 | | | 0.058 |
| 618 | 0.50 | | | 5 | 0.1 |
| 619 | 0.20 | | | 2.5 | 0.038 |
| 620 | 0.30 | | | 2 | 0.24 |
| 621 | 3.50 | 355.5 | | | 0.225 |
| 622 | 8.00 | 39 | | | 0.096 |
| 623 | 8.50 | 296.5 | | | 0.060 |
| 624 | 2.10 | 23 | | | 0.057 |
| 625 | 0.20 | 4.7 | | | 0.001 |
| 626 | 3.00 | 470 | | | 0.066 |
| 627 | 0.60 | 4.65 | 28.5 | 4.5 | 0.001 |
| 628 | 11.00 | 2727 | | | 0.150 |
| 629 | 1.30 | 25.5 | | | 0.050 |
| 630 | 1.15 | 157 | | | 0.200 |
| 631 | 5.00 | 54.5 | | | 0.002 |
| 632 | 1.90 | 246 | | | 0.049 |
| 633 | 3.87 | 1909.5 | | | 0.225 |
| 634 | 2.10 | 16.5 | | | 0.019 |
| 635 | 0.50 | 2.5 | | | 0.005 |
| 636 | 4.00 | 1006 | | | 0.165 |
| 637 | 2.20 | 18.5 | | | 0.035 |
| 638 | 3.00 | 214 | | | 0.008 |
| 639 | 0.33 | 3.42 | 5 | 1.45 | 0.012 |
| 640 | 488.00 | 6044 | | | 1.450 |
| 641 | 1.60 | 8 | | | 0.010 |
| 642 | 0.95 | 8 | | | 2.600 |
| 643 | 0.90 | 4 | | | >4.000 |
| 644 | 0.30 | 2.5 | | | 0.033 |
| 645 | 1.80 | 55 | | | 0.185 |
| 646 | 2.50 | 68 | | | 0.013 |
| 647 | 10.00 | 4849 | | | 1.260 |
| 648 | 2.95 | 37 | | | 0.009 |
| 649 | 0.40 | 19 | | | 0.031 |
| 650 | 0.40 | 3 | | | 0.031 |
| 651 | 0.90 | 19.66 | | | 0.783 |
| 652 | 3.50 | 18 | | | 0.135 |
| 653 | 4.00 | 21 | | | 0.125 |
| 654 | 1.50 | 7.5 | | | 0.088 |
| 655 | 0.60 | 7.5 | | | 0.175 |
| 656 | 0.95 | 52 | | | 0.035 |
| 657 | 0.40 | 3.5 | | | 0.084 |
| 658 | 0.30 | 2.15 | | | 2.450 |
| 659 | 0.30 | 2.2 | | | 0.015 |
| 660 | 0.30 | 2.95 | | | 0.004 |
| 661 | 0.30 | 2 | | | 0.006 |
| 662 | 81.50 | 3069 | | | 1.480 |
| 663 | 5.73 | 42.33 | | | 0.710 |
| 664 | 2.05 | 63 | | | 0.018 |
| 665 | 5.50 | 127 | | | 0.039 |
| 666 | 0.80 | 4.5 | | | 0.034 |
| 667 | 1.87 | 11.33 | | | 0.153 |
| 668 | 2.87 | 12.33 | | | 0.295 |
| 669 | 0.60 | 29 | | | 0.004 |
| 670 | 3.50 | 20.3 | | | 0.133 |
| 671 | 4.00 | 25 | | | 0.220 |
| 672 | 10.50 | 179.5 | | | 0.027 |
| 673 | 12.50 | 61.5 | | | 0.060 |
| 674 | 10.00 | 686 | | | 0.350 |
| 675 | 9.00 | 40 | | | 0.370 |
| 676 | 0.30 | 3.25 | | | 0.007 |
| 677 | 19.00 | 143.5 | | | 0.595 |
| 678 | 3.00 | 14.5 | | | 0.130 |
| 679 | 8.00 | 67.5 | | | 0.370 |
| 680 | 1920.00 | 6027 | | | 0.535 |
| 681 | 11.50 | 69.5 | | | 4.000 |
| 682 | 0.30 | 1.98 | | | 0.001 |
| 683 | 1.65 | 11 | | | 0.092 |
| 684 | 7.00 | 1718.5 | | | 0.019 |
| 685 | 0.80 | 115 | | | 0.016 |
| 686 | 0.70 | 5 | | | 0.110 |
| 687 | 1.10 | 44 | | | 0.087 |
| 688 | 0.30 | 1.95 | | | 0.006 |
| 689 | 0.20 | 2.5 | | | 0.004 |
| 690 | 0.20 | 2.75 | | | 0.004 |
| 691 | 0.25 | 2.1 | | | 0.001 |
| 692 | 7.00 | 104.66 | | | 0.008 |
| 693 | 0.75 | 5.07 | | | <0.001 |
| 694 | 4.00 | 95 | | | 0.155 |
| 695 | 4.00 | >5500.00 | | | 0.330 |
| 696 | 3.00 | 48 | | | 0.005 |
| 697 | 0.25 | 1.4 | | | 0.001 |
| 698 | 0.30 | 3 | | | 0.001 |
| 699 | 0.40 | 3 | | | 0.005 |
| 700 | 0.40 | 3 | | | 0.002 |
| 701 | 0.30 | 3 | | | 0.001 |
| 702 | 0.30 | 3 | | | 0.001 |
| 703 | 0.30 | 2.5 | | | 0.003 |
| 704 | 0.60 | 4 | | | 0.004 |
| 705 | 5.50 | 25.5 | | | 0.026 |
| 706 | 1.30 | 10 | | | 0.010 |
| 707 | 8.00 | 122.5 | | | 0.085 |
| 708 | 42.00 | 351.5 | | | 0.115 |
| 709 | 47.00 | 248 | | | 0.043 |
| 710 | 0.20 | 2.35 | 0.7 | 0.6 | <0.001 |
| 711 | 0.40 | 2.25 | | | <0.001 |
| 712 | 23.00 | 245.5 | | | 0.240 |
| 713 | 14.00 | 70.5 | | | 0.045 |
| 714 | 0.55 | 3.7 | | | <0.001 |
| 715 | 0.50 | 2.55 | | | 0.001 |
| 716 | 0.80 | 3.65 | | | 0.023 |
| 717 | 2.65 | 8 | | | 0.007 |
| 718 | 2.75 | 9.5 | | | 0.002 |
| 719 | 3.10 | 41.5 | | | 0.275 |
| 720 | 0.40 | 1.75 | | | 0.002 |
| 721 | 0.60 | 2.25 | | | 0.001 |
| 722 | 1.25 | 5 | | | 0.002 |
| 723 | 1.00 | 4.1 | | | 0.003 |
| 724 | 4.70 | 30.66 | | | 0.020 |
| 725 | 13.00 | 74 | | | 0.530 |
| 726 | 1.37 | 13.66 | | | 0.018 |
| 727 | 1.45 | 6.25 | | | 0.048 |
| 728 | 0.95 | 3.55 | | | 0.007 |
| 729 | 0.35 | 2.9 | | | 0.001 |
| 730 | 0.65 | 1.9 | | | 0.003 |
| 731 | 0.55 | 1.5 | | | 0.003 |
| 732 | 0.33 | 1.9 | | | 0.001 |
| 733 | 0.30 | 3 | | | <0.001 |

Throughout this application, various publications are referenced. The disclosures of these publications in their entireties are hereby incorporated by reference into this application in order to more fully describe the state of the art as known to those skilled therein as of the date of the invention described and claimed herein.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1) A compound of Formula I: 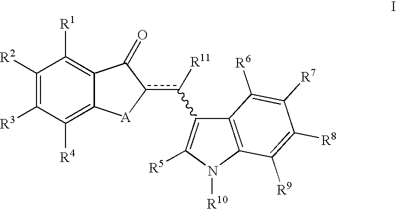
or a geometric isomer thereof or a pharmaceutically acceptable salt thereof wherein
A is oxygen or sulfur; [symbol]
(dashed line) represents an optional second carbon-to-carbon bond;
R¹, R², R³, and R⁴ are each independently H; C₁-C₆alkoxy optionally substituted with from 1 to 3 substituents independently selected from H₂N—, C₁-C₆aminoalkyl-, and di(C₁-C₆alkyl)amino-; C₁-C₆alkyl; (C₁-C₆alkoxy)carbonyl; C₆-C₁₄aryl optionally substituted with from 1 to 3 substituents independently selected from R¹²C(O)NH—; R¹⁴OC(O)NR¹²—, H₂N—, C₁-C₆aminoalkyl-, and di(C₁-C₆alkyl)amino-; C₁-C₉heteroaryl optionally substituted with from 1 to 3 substituents independently selected from R¹²C(O)NH—, R¹⁴OC(O)NR¹²—, H₂N—, C₁-C₆aminoalkyl-, and di(C₁-C₆alkyl)amino-; HO₂C—; C₁-C₆hydroxylalkyl-; R¹²R¹³N—; R¹²R¹³NC(O)—; C₁-C₉heterocyclyl-C(O)—; R¹²R¹³NC(O)NH—; R¹²R¹³NC(S)NH—; R¹²R¹³ NC(O)O—; C₁-C₉heterocyclyl-C(O)NH—; R¹²C(O)NH—; R¹⁴OC(O)NR¹²—; R¹⁴OC(O)NHC(O)NH—; R¹²S—; R¹²S(O)—; R¹²S(O)₂—; R¹²S(O)₂—O—; R¹²C(O)—; R¹²S(O)₂—NR¹²—; R¹²R¹³NS(O)₂—; C₂-C₆alkenyl; C₂-C₆alkynyl; C₁-C₉heterocyclyl-optionally substituted by C₁-C₆alkyl; halo; CN; NO₂; or hydroxyl;
R¹² and R¹³ are each independently: a) H; b) C₁-C₆alkyl optionally substituted with from 1 to 3 substituents independently selected from: i) H₂N—, ii) C₁-C₆aminoalkyl-, iii) di(C₁-C₆alkyl)amino-, iv) C₁-C₉heteroaryl, v) halo, vi) hydroxyl, vii) C₁-C₆alkoxy optionally substituted with from 1 to 3 substituents independently selected from: A) hydroxyl, B) C₁-C₆alkoxy, C) H₂N—, D) C₁-C₆aminoalkyl-, and E) di(C₁-C₆alkyl)amino-, viii) C₁-C₉heterocyclyl, ix) di(C₁-C₆alkyl)amino-optionally substituted with from 1 to 3 substituents independently selected from: A) hydroxyl, B) C₁-C₆alkoxy, C) H₂N—, D) C₁-C₆aminoalkyl-, and E) di(C₁-C₆alkyl)amino-; c) C₂-C₆alkenyl; d) C₂-C₆alkynyl; e) C₁-C₉heterocyclyl-optionally substituted by C₁-C₆alkyl; f) perfluoro(C₁-C₆)alkyl; g) C₁-C₉heteroaryl optionally substituted with from 1 to 3 substituents independently selected from: i) C₁-C₆alkyl optionally substituted with a substituent selected from: A) hydroxyl, B) H₂N—, C) C₁-C₆aminoalkyl-, D) di(C₁-C₆alkyl)amino-, and E) C₁-C₉heterocyclyl-optionally substituted by C₁-C₆alkyl, ii) halo, iii) hydroxyl, iv) C₁-C₆alkoxy, v) H₂N—, vi) C₁-C₆aminoalkyl-, vii) di(C₁-C₆alkyl)amino-, viii) O₂N—, ix) H₂NSO₂—, x) HO₂C—, xi) (C₁-C₆alkoxy)carbonyl, xii) (C₁-C₆alkoxy)carbonyl-NH—, xiii) Q-Z-, wherein Z is A) —O—, B) —N(CH₃)—, C) —NH—, D) —C(O)N(CH₃)—, E) —C(O)NH—, F) —N(CH₃)C(O)—, G) —NHC(O)—, H) —NHSO₂—, I) —N(CH₃)SO₂— J) —SO₂NH—, K) —SO₂N(CH₃)—, L) —NHC(O)NH—, M) —S—, N) —S(O)—, O) S(O)₂, or P) is absent, and Q is selected from: A) C₆-C₁₄aryl, B) C₁-C₉heteroaryl, C) C₁-C₉heterocyclyl-optionally substituted with from 1 to 3 substituents independently selected from: 1) C₁-C₆alkyl, 2) C₁-C₆hydroxylalkyl-, 3) di(C₁-C₆alkyl)amino-, and 4) perfluoro(C₁-C₆)alkyl; D) C₃-C₈cycloalkyl, E) C₁-C₆alkyl, F) C₂-C₆alkenyl, G) C₂-C₆alkynyl, H) (C₁-C₆alkyl)amino-C₁-C₆alkylene-, I) di(C₁-C₆alkyl)amino-C₁-C₆alkylene-, J) (C₆-C₁₄aryl)alkyl, K) (C₁-C₉heteroaryl)alkyl, or L) heterocyclyl(C₁-C₆alkyl), xiv) HC(O)—, xv) (C₁-C₆alkyl)C(O)—, xvi) (C₃-C₈cycloalkyl)C(O)—, xvii) (C₁-C₉heterocyclyl)C(O)— optionally substituted with A) C₁-C₆alkyl, B) C₁-C₆hydroxylalkyl-, C) di(C₁-C₆alkyl)amino-, or D) perfluoro(C₁-C₆)alkyl; h) C₆-C₁₄aryl optionally substituted with from 1 to 3 substituents independently selected from: i) C₁-C₆alkyl optionally substituted with a substituent selected from: A) hydroxyl, B) H₂N—, C) C₁-C₆aminoalkyl-, D) di(C₁-C₆alkyl)amino-, and E) C₁-C₉heterocyclyl-optionally substituted by C₁-C₆alkyl, ii) halo, iii) hydroxyl, iv) C₁-C₆alkoxy, v) H₂N—, vi) C₁-C₆aminoalkyl-, vii) di(C₁-C₆alkyl)amino-, viii) O₂N—, ix) H₂NSO₂—, x) HO₂C—, xi) (C₁-C₆alkoxy)carbonyl, xii) (C₁-C₆alkoxy)carbonyl-NH—, xiii) Q-Z-, wherein Z is A) —O—, B) —N(CH₃)—, C) —NH—, D) —C(O)N(CH₃)—, E) —C(O)NH—, F) —N(CH₃)C(O)—, G) —NHC(O)—, H) —NHSO₂—, I) —N(CH₃)SO₂— J) —SO₂NH—, K) —SO₂N(CH₃)—, L) —NHC(O)NH—, M) —S—, N) —S(O)—, O) S(O)₂, or P) is absent, and Q is selected from: A) C₆-C₁₄aryl, B) C₁-C₉heteroaryl, C) C₁-C₉heterocyclyl-optionally substituted with from 1 to 3 substituents independently selected from: 1) C₁-C₆alkyl, 2) C₁-C₆hydroxylalkyl-, 3) di(C₁-C₆alkyl)amino-, and 4) perfluoro(C₁-C₆)alkyl; D) C₃-C₈cycloalkyl, E) C₁-C₆alkyl, F) C₂-C₆alkenyl, G) C₂-C₆alkynyl, H) (C₁-C₆alkyl)amino-C₁-C₆alkylene-, I) di(C₁-C₆alkyl)amino-C₁-C₆alkylene-, J) (C₆-C₁₄aryl)alkyl, K) (C₁-C₉heteroaryl)alkyl, or L) heterocyclyl(C₁-C₆alkyl), xiv) HC(O)—, xv) (C₁-C₆alkyl)C(O)—, xvi) (C₃-C₈cycloalkyl)C(O)—, xvii) (C₁-C₉heterocyclyl)C(O)— optionally substituted with A) C₁-C₆alkyl, B) C₁-C₆hydroxylalkyl-, C) di(C₁-C₆alkyl)amino-, or D) perfluoro(C₁-C₆)alkyl; or i) C₃-C₈cycloalkyl;
R¹⁴ is independently C₁-C₆alkyl, C₁-C₆hydroxylalkyl-, or C₆-C₁₄aryl;
R⁵ is H; C₁-C₆alkyl optionally substituted with from 1 to 3 substituents independently selected from halo, H₂N—, C₁-C₆aminoalkyl-, di(C₁-C₆alkyl)amino-, (CH₃)₂N(CH₂)₂N(CH₃)—, —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆alkoxy, C₁-C₆alkyl, —C(O)OH, (C₁-C₆alkoxy)carbonyl, —C(O)(C₁-C₆alkyl), C₆-C₁₄aryl, C₁-C₉heteroaryl, C₃-C₈cycloalkyl, C₁-C₆haloalkyl-, C₁-C₆aminoalkyl-, —OC(O)(C₁-C₆alkyl), C₁-C₆carboxyamidoalkyl-, and —NO₂; C₆-C₁₄aryl optionally substituted with from 1 to 3 substituents independently selected from C₁-C₆alkoxy, C₁-C₆alkyl, (C₆-C₁₄aryl)alkyl-O—, C₃-C₈cycloalkyl, di(C₁-C₆alkyl)amino-C₁-C₆alkylene-, C₁-C₆ perfluoroalkyl-, halo, C₁-C₆haloalkyl-, hydroxyl, C₁-C₆hydroxylalkyl-, H₂N—, C₁-C₆aminoalkyl-, di(C₁-C₆alkyl)amino-, —COOH, —C(O)O—(C₁-C₆alkyl), —OC(O)(C₁-C₆alkyl), (C₁-C₆alkyl)carboxyamido, —C(O)NH₂, (C₁-C₆alkyl)amido-, —O—CH₂CH₂OCH₃, —O—CH₂CH₂OCH₂CH₃, —O—CH₂CH₂OCH₂CH₂OCH₃, —O—CH₂CH₂OCH₂CH₂OCH₂CH₃, and —NO₂; C₃-C₈cycloalkyl; halo; C₁-C₉heteroaryl optionally substituted with from 1 to 3 substituents independently selected from C₁-C₆alkoxy, C₁-C₆alkyl, C₃-C₈cycloalkyl, di(C₁-C₆alkyl)amino-C₁-C₆alkylene-, C₁-C₆ perfluoroalkyl-, halo, C₁-C₆haloalkyl-, hydroxyl, C₁-C₆hydroxylalkyl-, H₂N—, C₁-C₆aminoalkyl-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, (C₁-C₆alkyl)amido-, —O—CH₂CH₂OCH₃, —O—CH₂CH₂OCH₂CH₃, —O—CH₂CH₂OCH₂CH₂OCH₃, —O—CH₂CH₂OCH₂CH₂OCH₂CH₃, and —NO₂; C₁-C₉heterocyclyl-optionally substituted by C₁-C₆alkyl; C₁-C₆heterocyclylalkyl optionally substituted with from 1 to 3 C₁-C₆alkyl groups; C₁-C₆ perfluoroalkyl-; R¹⁵R¹⁶NC(O)—; CN; (C₁-C₆alkoxy)carbonyl; or CO₂H;
R¹⁵ and R¹⁶ are each independently H; C₁-C₆alkyl optionally substituted with from 1 to 3 substituents independently selected from hydroxyl, H₂N—, —NH(C₁-C₆alkyl), —N(C₁-C₆alkyl)(C₁-C₆alkyl), and C₁-C₉heteroaryl; C₁-C₉heteroaryl; C₆-C₁₄aryl optionally substituted with from 1 to 3 substituents independently selected from C₁-C₆alkyl, halo, and perfluoro(C₁-C₆)alkyl; C₃-C₈cycloalkyl;
or R¹⁵ and R¹⁶, when taken together with the nitrogen to which they are attached, form a 3- to 7-membered heterocycle, which heterocycle may optionally comprise 1 or 2 additional heteroatoms independently selected from —N(H)—, —N(C₁-C₆alkyl)-, —N(C₆-C₁₄aryl)-, —S—, —SO—, —S(O)₂, and —O—;
R⁶, R⁷, R⁸, and R⁹ are independently selected from:
a) H; b) C₁-C₆alkoxy optionally substituted by C₁-C₆alkoxy; c) C₁-C₆alkyl optionally substituted with from 1 to 3 substituents independently selected from: i) C₆-C₁₄aryl; ii) H₂N—, iii) C₁-C₆aminoalkyl-, iv) di(C₁-C₆alkyl)amino-, and v) C₁-C₉heterocyclyl optionally substituted by C₁-C₆alkyl; d) C₂-C₆alkenyl optionally substituted with from 1 to 3 substituents independently selected from: i) C₆-C₁₄aryl; ii) H₂N—, iii) C₁-C₆aminoalkyl-, iv) di(C₁-C₆alkyl)amino-, and v) C₁-C₉heterocyclyl optionally substituted by C₁-C₆alkyl; e) C₂-C₆alkynyl optionally substituted with from 1 to 3 substituents independently selected from: i) C₆-C₁₄aryl; ii) H₂N—, iii) C₁-C₆aminoalkyl-, iv) di(C₁-C₆alkyl)amino-, and v) C₁-C₉heterocyclyl optionally substituted by C₁-C₆alkyl; f) (C₁-C₆alkyl)amido-; g) C₁-C₆alkylcarboxy; h) (C₁-C₆alkyl)carboxyamido; i) (C₁-C₆alkyl)SO₂—; j) C₆-C₁₄aryl optionally substituted with from 1 to 3 substituents independently selected from: i) C₁-C₈acyl, ii) C₁-C₆alkyl, which is optionally substituted with from 1 to 3 substituents independently selected from: A) H₂N—, B) C₁-C₆aminoalkyl-, C) di(C₁-C₆alkyl)amino-, and D) C₁-C₉heterocyclyl-optionally substituted by C₁-C₆alkyl, iii) (C₁-C₆alkyl)amido-, iv) (C₁-C₆alkyl)carboxyl, v) (C₁-C₆alkyl)carboxyamido, vi) C₁-C₆alkoxy optionally substituted by C₁-C₆alkoxy or C₁-C₉heteroaryl, vii) (C₁-C₆alkoxy)carbonyl, viii) (C₆-C₁₄aryl)oxy, ix) C₃-C₈cycloalkyl, x) halo, xi) C₁-C₆haloalkyl-, xii) C₁-C₉heterocyclyl optionally substituted by C₁-C₆alkyl or C₁-C₆hydroxylalkyl-, xiii) hydroxyl, xiv) C₁-C₆hydroxylalkyl-, xv) C₁-C₆ perfluoroalkyl-, xvi) C₁-C₆ perfluoroalkyl-O—, xvii) R¹⁷R¹⁸N—, xviii) C₁-C₉heterocyclyl-optionally substituted by C₁-C₆alkyl, xix) CN, xx)-COOH, xxi) R¹⁷R¹⁸NC(O)—, xxii) C₁-C₉heterocyclyl-C(O)—, xxiii) R¹⁷C(O)NH—, xxiv) R¹⁷R¹⁸NS(O)₂—, xxv) C₁-C₉heterocyclyl-S(O)₂—, xxvi) R¹⁷R¹⁸NC(O)NH—, xxvii) C₁-C₉heterocyclyl-C(O)NH—, xxviii) R¹⁹OC(O)NH—, xxix) (C₁-C₆alkyl)S(O)₂NH—, xxx) R¹⁹S(O)₂—, xxxi) —C(═N—(OR¹⁷))—(NR¹⁷R¹⁸), and xxxii) —NO₂; k) (C₆-C₁₄aryl)alkyl-O—; I) (C₆-C₁₄aryl)oxy; m) halogen; n) C₁-C₉heteroaryl optionally substituted with from 1 to 3 substituents independently selected from: i) C₁-C₈acyl, ii) C₁-C₆alkyl, which is optionally substituted with from 1 to 3 substituents independently selected from: A) H₂N—, B) C₁-C₆aminoalkyl-, C) di(C₁-C₆alkyl)amino-, and D) C₁-C₉heterocyclyl-optionally substituted by C₁-C₆alkyl, iii) (C₁-C₆alkyl)amido-, iv) (C₁-C₆alkyl)carboxyl, v) (C₁-C₆alkyl)carboxyamido, vi) C₁-C₆alkoxy optionally substituted by C₁-C₆alkoxy or C₁-C₉heteroaryl, vii) (C₁-C₆alkoxy)carbonyl, viii) (C₆-C₁₄aryl)oxy, ix) C₃-C₈cycloalkyl, x) halo, xi) C₁-C₆haloalkyl-, xii) C₁-C₉heterocyclyl optionally substituted by C₁-C₆alkyl or C₁-C₆hydroxylalkyl-, xiii) hydroxyl, xiv) C₁-C₆hydroxylalkyl-, xv) C₁-C₆ perfluoroalkyl-, xvi) C₁-C₆ perfluoroalkyl-O—, xvii) R¹⁷R¹⁸N—, xviii) C₁-C₉heterocyclyl-optionally substituted by C₁-C₆alkyl, xix) CN, xx)-COOH, xxi) R¹⁷R¹⁸NC(O)—, xxii) C₁-C₉heterocyclyl-C(O)—, xxiii) R¹⁷C(O)NH—, xxiv) R¹⁷R¹⁸NS(O)₂—, xxv) C₁-C₉heterocyclyl-S(O)₂—, xxvi) R¹⁷R¹⁸NC(O)NH—, xxvii) C₁-C₉heterocyclyl-C(O)NH—, xxviii) R¹⁹OC(O)NH—, xxix) (C₁-C₆alkyl)S(O)₂NH—, xxx) R¹⁹S(O)₂—, xxxi) —C(═N—(OR¹⁷))—(NR¹⁷R¹⁸), and xxxii) —NO₂; o) hydroxyl; p) H₂N—; q) R¹⁷C(O)NH—; r) C₁-C₆alkylS(O)₂—O— s) C₁-C₉heterocyclyl optionally substituted with from 1 to 3 substituents independently selected from: i) C₁-C₆alkyl, which is optionally substituted with from 1 to 3 substituents independently selected from: A) H₂N—, B) C₁-C₆aminoalkyl-, and C) di(C₁-C₆alkyl)amino-, ii) R¹⁷R¹⁸NC(O)—, iii) hydroxyl, and iv) R¹⁷R¹⁸N—; t) C₁-C₆ perfluoroalkyl-; u) CN; v) (C₁-C₆alkoxy)carbonyl; w) CO₂H; and x) NO₂;
or R⁷ and R⁸ when taken together can be replaced by an alkylenedioxy group so that the alkylenedioxy group, when taken together with the two carbon atoms to which it is attached, forms a 5- to 7-membered heterocycle containing two oxygen atoms;
R¹⁷ and R¹³ are each independently H; C₁-C₆alkyl optionally substituted with from 1 to 3 substituents independently selected from C₁-C₆alkoxy, H₂N—, C₁-C₆aminoalkyl-, di(C₁-C₆alkyl)amino-, C₆-C₁₄aryl, C₁-C₉heterocyclyl-optionally substituted by C₁-C₆alkyl, and C₁-C₉heteroaryl; C₁-C₆alkoxy; C₁-C₉heteroaryl; hydroxyl; C₆-C₁₄aryl optionally substituted with from 1 to 3 substituents independently selected from C₁-C₆alkyl, halo, and perfluoro(C₁-C₆)alkyl; and C₃-C₈cycloalkyl;
or R¹⁷ and R¹⁸ when taken together with the nitrogen to which they are attached form a 3- to 7-membered heterocycle, which heterocycle may optionally comprise 1 or 2 additional heteroatoms independently selected from —N(H)—, —N(C₁-C₆alkyl)-, —N(C₆-C₁₄aryl)-, —S—, —SO—, —S(O)₂, or —O—; —N(H)—, —N(C₁-C₆alkyl)-, —N(C₁-C₆hydroxylalkyl)-, —N(C₁-C₆alkylene-di(C₁-C₆alkyl)amino)-, —N(C₆-C₁₄aryl)-, —S—, —SO—, —S(O)₂, and —O—;
R¹⁹ is C₁-C₆alkyl or C₆-C₁₄aryl;
R¹⁰ is C₁-C₆alkyl substituted with from 1 to 3 substituents independently selected from halogen, hydroxyl, C₁-C₆hydroxylalkyl-NH—, C₁-C₆hydroxylalkyl-N(CH₃)—, H₂N—, C₁-C₆aminoalkyl-, di(C₁-C₆alkyl)amino-, di(C₁-C₆alkyl)amino-(C₁-C₆alkylene)-NH—, di(C₁-C₆alkyl)amino-(C₁-C₆alkylene)-N(CH₃)—, —N(C₁-C₃alkyl)C(O)(C₁-C₆alkyl), —NHC(O)(C₁-C₆alkyl), —C(O)N(C₁-C₆alkyl)(C₁-C₆alkyl), —CN, C₁-C₆alkoxy, C₃-C₈cycloalkyl, C₁-C₆haloalkyl-, C₁-C₆aminoalkyl-, —OC(O)(C₁-C₆alkyl), —C(O)OH, (C₁-C₆alkoxy)carbonyl, —C(O)(C₁-C₆alkyl), —NHC(O)H, —C(O)NH₂, and —NO₂; C₂-C₁₀alkenyl; C₆-C₁₄aryl; (C₆-C₁₄aryl)alkyl; C₃-C₈cycloalkyl; C₁-C₉heteroaryl; (C₁-C₉heteroaryl)alkyl; C₁-C₆carboxyamidoalkyl-; or C₁-C₆heterocyclylalkyl group optionally substituted with from 1 to 3 substituents independently selected from halogen, H₂N—, C₁-C₆aminoalkyl-, di(C₁-C₆alkyl)amino-, —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆hydroxylalkyl-, C₁-C₆alkoxy, C₁-C₆alkyl, —C(O)OH, (C₁-C₆alkoxy)carbonyl, —C(O)(C₁-C₆alkyl), 4- to 7-membered monocyclic heterocycle, C₆-C₁₄aryl, C₁-C₉heteroaryl, C₁-C₆heterocyclylalkyl, and C₃-C₈cycloalkyl;
or R¹⁰ is H, C₁-C₆alkyl, or C₁-C₈acyl provided that:
1) R² is not hydrogen, or 2) R³ is not hydroxyl, C₁-C₆alkoxy, or (C₁-C₆alkoxy)carbonyl, or 3) R⁵ is not H, C₁-C₆alkyl, or C₃-C₈cycloalkyl, or 4) any of R⁶, R⁷ R⁸ or R⁹ is: a) C₁-C₆alkoxy substituted by C₁-C₆alkoxy; b) C₁-C₆alkyl optionally substituted by C₆-C₁₄aryl; c) (C₁-C₆alkyl)SO₂—; d) C₆-C₁₄aryl optionally substituted with from 1 to 3 substituents independently selected from: i) C₁-C₈acyl, ii) C₁-C₆alkyl, which is optionally substituted with from 1 to 3 substituents independently selected from: A) H₂N—, C₁-C₆aminoalkyl-, B) di(C₁-C₆alkyl)amino-, and C) C₁-C₉heterocyclyl-optionally substituted by C₁-C₆alkyl, iii) (C₁-C₆alkyl)amido-, iv) (C₁-C₆alkyl)carboxyl, v) (C₁-C₆alkyl)carboxyamido, vi) C₁-C₆alkoxy optionally substituted by C₁-C₆alkoxy or C₁-C₉heteroaryl, vii) vii) (C₁-C₆alkoxy)carbonyl, viii) (C₆-C₁₄aryl)oxy, ix) C₃-C₈cycloalkyl, x) halo, xi) C₁-C₆haloalkyl-, xii)) C₁-C₉heterocyclyl optionally substituted by C₁-C₆alkyl or C₁-C₆hydroxylalkyl-, xiii) hydroxyl, xiv) C₁-C₆hydroxylalkyl-, xv) C₁-C₆ perfluoroalkyl-, xvi) C₁-C₆ perfluoroalkyl-O—, xvii) R¹⁷R¹¹N—, xviii) C₁-C₉heterocyclyl-optionally substituted by C₁-C₆alkyl, xix) CN, xx)-COOH, xxi) R¹⁷R¹⁸NC(O)—, xxii) C₁-C₉heterocyclyl-C(O)—, xxiii) R¹⁷C(O)NH—, xxiv) R¹⁷R¹⁸NS(O)₂—, xxv) C₁-C₉heterocyclyl-S(O)₂—, xxvi) R¹⁷R¹⁸NC(O)NH—, xxvii) C₁-C₉heterocyclyl-C(O)NH—, xxviii) R¹⁹OC(O)NH—, xxix (C₁-C₆alkyl)S(O)₂NH—, xxx) R¹⁹S(O)₂—, xxxi) —C(═N—(OR¹⁷))—(NR¹⁷R¹⁸), and xxxi) —NO₂; e) (C₆-C₁₄aryl)alkyl-O—; f) halo; C₁-C₉heteroaryl optionally substituted with from 1 to 3 substituents independently selected from: i) C₁-C₈acyl, ii) C₁-C₆alkyl, which is optionally substituted with from 1 to 3 substituents independently selected from: A) H₂N—, C₁-C₆aminoalkyl-, B) di(C₁-C₆alkyl)amino-, and C) C₁-C₉heterocyclyl-optionally substituted by C₁-C₆alkyl, iii) (C₁-C₆alkyl)amido-, iv) (C₁-C₆alkyl)carboxyl, v) (C₁-C₆alkyl)carboxyamido, vi) C₁-C₆alkoxy optionally substituted by C₁-C₆alkoxy or C₁-C₉heteroaryl, vii) vii) (C₁-C₆alkoxy)carbonyl, viii) (C₆-C₁₄aryl)oxy, ix) C₃-C₈cycloalkyl, x) halo, xi) C₁-C₆haloalkyl-, xii)) C₁-C₉heterocyclyl optionally substituted by C₁-C₆alkyl or C₁-C₆hydroxylalkyl-, xiii) hydroxyl, xiv) C₁-C₆hydroxylalkyl-, xv) C₁-C₆ perfluoroalkyl-, xvi) C₁-C₆ perfluoroalkyl-O—, xvii) R¹⁷R¹⁸N—, xviii) C₁-C₉heterocyclyl-optionally substituted by C₁-C₆alkyl, xix) CN, xx)-COOH, xxi) R¹⁷R¹⁸NC(O)—, xxii) C₁-C₉heterocyclyl-C(O)—, xxiii) R¹⁷C(O)NH—, xxiv) R¹⁷R¹⁸NS(O)₂—, xxv) C₁-C₉heterocyclyl-S(O)₂—, xxvi) R¹⁷R¹⁸NC(O)NH—, xxvii) C₁-C₉heterocyclyl-C(O)NH—, xxviii) R¹⁹OC(O)NH—, xxix (C₁-C₆alkyl)S(O)₂NH—, xxx) R¹⁹S(O)₂—, xxxi) —C(═N—(OR¹⁷))—(NR¹⁷R¹⁸), and xxxi) —NO₂; h) hydroxyl; i) C₁-C₉heterocyclyl optionally substituted with from 1 to 3 substituents independently selected from: i) C₁-C₆alkyl, which is optionally substituted with from 1 to 3 substituents independently selected from: A) H₂N—, B) C₁-C₆aminoalkyl-, and C) di(C₁-C₆alkyl)amino-, ii) R¹⁷R¹⁸NC(O)—, iii) hydroxyl, and iv) R¹⁷R¹⁸N—; j) C₁-C₆ perfluoroalkyl-; k) CN; I) (C₁-C₆alkoxy)carbonyl; m) CO₂H; and n) NO₂; or 5) any of R⁶, R⁸ or R⁹ is C₁-C₆alkoxy;
R¹¹ is H or C₁-C₆alkyl;
with the proviso (1) that R¹, R², R³, R⁴, R⁶, R⁷, R⁸, and R⁹ cannot simultaneously be H; and (2) that 4-hydroxy-6-methyl-2-[(1-methyl-1H-indol-3-yl)methylene]-(2H)-benzofuranone, 2-[(5-bromo-1H-indol-3-yl)methylene]-benzo[b]thiophen-3(2H)-one, (2Z)-5-chloro-2-[(2-phenyl-1H-indol-3-yl)methylene]-1-benzothiophen-3(2H)-one, and 2-[(7-ethyl-1H-indol-3-yl)methylene]-benzo[b]thiophen-3(2H)-one are excluded.

2) A compound of claim 1 wherein, A is oxygen.

3) A compound of claim 2 wherein, R¹ is H.

4) A compound of claim 3 wherein, R² is R¹²R¹³NC(O)NH—.

5) A compound of claim 4 wherein, R¹² is C₆-C₁₄aryl substituted with di(C₁-C₆alkyl)amino-C₂-C₆alkylene-N(C₁-C₆alkyl)C(O)—.

6) A compound of claim 5 wherein, R³ is H.

7) A compound of claim 6 wherein, R⁴ is H.

8) A compound of claim 7 wherein, R⁵ is C₁-C₉heteroaryl independently substituted with from 1 to 3 C₁-C₆alkyl substituents.

9) A compound of claim 8 wherein, R⁵ is 1,3,5-trimethyl-1H-pyrazol-4-yl.

10) A compound of claim 9 wherein, R⁶ is H.

11) A compound of claim 10 wherein, R⁷ is C₁-C₆alkoxy.

12) A compound of claim 11 wherein, R⁷ is CH₃O—.

13) A compound of claim 12 wherein, R⁸ is H.

14) A compound of claim 13 wherein, R⁹ is halogen.

15) A compound of claim 14 wherein, R¹⁰ is H.

16) A compound of claim 15 wherein, R¹¹ is H.

17) A compound selected from the group consisting of:
2-(1H-indol-3-ylmethylene)-1-benzofuran-3(2H)-one;
2-[(2-phenyl-1H-indol-3-yl)methylene]-1-benzofuran-3(2H)-one;
2-[(1-methyl-2-phenyl-1H-indol-3-yl)methylene]-1-benzofuran-3(2H)-one;
2-[(1-methyl-1H-indol-3-yl)methylene]-1-benzofuran-3(2H)-one;
6-hydroxy-2-[(2-phenyl-1H-indol-3-yl)methylene]-1-benzofuran-3(2H)-one;
6-hydroxy-2-[(1-methyl-2-phenyl-1H-indol-3-yl)methylene]-1-benzofuran-3(2H)-one;
2-(1H-indol-3-ylmethylene)-7-methoxy-1-benzofuran-3(2H)-one;
7-methoxy-2-[(2-phenyl-1H-indol-3-yl)methylene]-1-benzofuran-3(2H)-one;
7-methoxy-2-[(1-methyl-2-phenyl-1H-indol-3-yl)methylene]-1-benzofuran-3(2H)-one;
7-methoxy-2-[(1-methyl-1H-indol-3-yl)methylene]-1-benzofuran-3(2H)-one;
4,6-dihydroxy-2-[(2-phenyl-1H-indol-3-yl)methylene]-1-benzofuran-3(2H)-one;
4,6-dihydroxy-2-[(1-methyl-2-phenyl-1H-indol-3-yl)methylene]-1-benzofuran-3(2H)-one;
(2Z)-2-{[2-(4-chlorophenyl)-1H-indol-3-yl]methylene}-1-benzofuran-3(2H)-one;
(2Z)-2-{[2-(2-naphthyl)-1H-indol-3-yl]methylene}-1-benzofuran-3(2H)-one;
(2Z)-2-{[2-(4-fluorophenyl)-1H-indol-3-yl]methylene}-1-benzofuran-3(2H)-one;
(2Z)-2-[(2-methyl-5-nitro-1H-indol-3-yl)methylene]-1-benzofuran-3(2H)-one;
(2Z)-2-[(2-methyl-1H-indol-3-yl)methylene]-1-benzofuran-3(2H)-one;
(2Z)-2-[(6-methyl-1H-indol-3-yl)methylene]-1-benzofuran-3(2H)-one;
(2Z)-2-[(7-methyl-1H-indol-3-yl)methylene]-1-benzofuran-3(2H)-one;
(2Z)-2-[(5-bromo-1H-indol-3-yl)methylene]-1-benzofuran-3(2H)-one;
(2Z)-2-[(1-benzyl-1H-indol-3-yl)methylene]-1-benzofuran-3(2H)-one;
(2Z)-2-{[2-(4-chlorophenyl)-1H-indol-3-yl]methylene}-6-hydroxy-1-benzofuran-3(2H)-one;
(2Z)-6-hydroxy-2-{[2-(2-naphthyl)-1H-indol-3-yl]methylene}-1-benzofuran-3(2H)-one;
(2Z)-2-{[2-(4-fluorophenyl)-1H-indol-3-yl]methylene}-6-hydroxy-1-benzofuran-3(2H)-one;
(2Z)-6-hydroxy-2-[(2-methyl-5-nitro-1H-indol-3-yl)methylene]-1-benzofuran-3(2H)-one;
(2Z)-6-hydroxy-2-[(6-methyl-1H-indol-3-yl)methylene]-1-benzofuran-3(2H)-one;
(2Z)-2-[(5-bromo-1H-indol-3-yl)methylene]-6-hydroxy-1-benzofuran-3(2H)-one;
(2Z)-2-[(1-benzyl-1H-indol-3-yl)methylene]-6-hydroxy-1-benzofuran-3(2H)-one;
(2Z)-2-{[5-(benzyloxy)-1H-indol-3-yl]methylene}-6-hydroxy-1-benzofuran-3(2H)-one;
(2Z)-2-{[2-(4-chlorophenyl)-1H-indol-3-yl]methylene}-7-methoxy-1-benzofuran-3(2H)-one;
(2Z)-7-methoxy-2-{[2-(2-naphthyl)-1H-indol-3-yl]methylene}-1-benzofuran-3(2H)-one;
(2Z)-2-{[2-(4-fluorophenyl)-1H-indol-3-yl]methylene}-7-methoxy-1-benzofuran-3(2H)-one;
(2Z)-7-methoxy-2-[(2-methyl-5-nitro-1H-indol-3-yl)methylene]-1-benzofuran-3(2H)-one;
(2Z)-7-methoxy-2-[(2-methyl-1H-indol-3-yl)methylene]-1-benzofuran-3(2H)-one;
(2Z)-7-methoxy-2-[(6-methyl-1H-indol-3-yl)methylene]-1-benzofuran-3(2H)-one;
(2Z)-2-[(5-bromo-1H-indol-3-yl)methylene]-7-methoxy-1-benzofuran-3(2H)-one;
(2Z)-2-[(1-benzyl-1H-indol-3-yl)methylene]-7-methoxy-1-benzofuran-3(2H)-one;
(2Z)-2-{[5-(benzyloxy)-1H-indol-3-yl]methylene}-7-methoxy-1-benzofuran-3(2H)-one;
(2Z)-2-{[2-(4-chlorophenyl)-1H-indol-3-yl]methylene}-4,6-dihydroxy-1-benzofuran-3(2H)-one;
(2Z)-4,6-dihydroxy-2-{[2-(2-naphthyl)-1H-indol-3-yl]methylene}-1-benzofuran-3(2H)-one;
(2Z)-2-{[2-(4-fluorophenyl)-1H-indol-3-yl]methylene}-4,6-dihydroxy-1-benzofuran-3(2H)-one;
(2Z)-4,6-dihydroxy-2-[(6-methyl-1H-indol-3-yl)methylene]-1-benzofuran-3(2H)-one;
(2Z)-4,6-dihydroxy-2-[(7-methyl-1H-indol-3-yl)methylene]-1-benzofuran-3(2H)-one;
(2Z)-2-[(5-bromo-1H-indol-3-yl)methylene]-4,6-dihydroxy-1-benzofuran-3(2H)-one;
(2Z)-2-[(1-benzyl-1H-indol-3-yl)methylene]-4,6-dihydroxy-1-benzofuran-3(2H)-one;
2-{[5-(benzyloxy)-1H-indol-3-yl]methylene}-4,6-dihydroxy-1-benzofuran-3(2H)-one;
(2Z)-6,7-dihydroxy-2-[(2-phenyl-1H-indol-3-yl)methylene]-1-benzofuran-3(2H)-one;
(2Z)-2-{[2-(4-chlorophenyl)-1H-indol-3-yl]methylene}-6,7-dihydroxy-1-benzofuran-3(2H)-one;
(2Z)-6,7-dihydroxy-2-{[2-(2-naphthyl)-1H-indol-3-yl]methylene}-1-benzofuran-3(2H)-one;
(2Z)-2-{[2-(4-fluorophenyl)-1H-indol-3-yl]methylene}-6,7-dihydroxy-1-benzofuran-3(2H)-one;
(2Z)-2-[(5-bromo-1H-indol-3-yl)methylene]-6,7-dihydroxy-1-benzofuran-3(2H)-one;
(2Z)-2-[(5-chloro-1H-indol-3-yl)methylene]-4,6-dihydroxy-1-benzofuran-3(2H)-one;
(2Z)-2-[(5-bromo-2-methyl-1H-indol-3-yl)methylene]-4,6-dihydroxy-1-benzofuran-3(2H)-one;
(2Z)-2-[(5-chloro-1H-indol-3-yl)methylene]-6,7-dihydroxy-1-benzofuran-3(2H)-one;
(2Z)-2-[(5-fluoro-1H-indol-3-yl)methylene]-6,7-dihydroxy-1-benzofuran-3(2H)-one;
(2Z)-6,7-dihydroxy-2-[(5-methyl-1H-indol-3-yl)methylene]-1-benzofuran-3(2H)-one;
(2Z)-2-[(5-bromo-2-methyl-1H-indol-3-yl)methylene]-6,7-dihydroxy-1-benzofuran-3(2H)-one;
(2Z)-7-hydroxy-2-[(2-phenyl-1H-indol-3-yl)methylene]-1-benzofuran-3(2H)-one;
(2Z)-2-{[2-(4-fluorophenyl)-1H-indol-3-yl]methylene}-7-hydroxy-1-benzofuran-3(2H)-one;
4,6-dihydroxy-2-[(5-methoxy-2-phenyl-1H-indol-3-yl)methylene]-1-benzofuran-3(2H)-one;
6,7-dihydroxy-2-[(5-methoxy-2-phenyl-1H-indol-3-yl)methylene]-1-benzofuran-3(2H)-one;
4,6-dihydroxy-2-[(2-pyridin-2-yl-1H-indol-3-yl)methylene]-1-benzofuran-3(2H)-one;
4-hydroxy-2-(1H-indol-3-ylmethylene)-1-benzofuran-3(2H)-one;
4-hydroxy-2-[(2-phenyl-1H-indol-3-yl)methylene]-1-benzofuran-3(2H)-one;
2-{[2-(4-chlorophenyl)-1H-indol-3-yl]methylene}-4-hydroxy-1-benzofuran-3(2H)-one;
2-[(5-bromo-1H-indol-3-yl)methylene]-4-hydroxy-1-benzofuran-3(2H)-one;
4-hydroxy-2-[(5-methoxy-2-phenyl-1H-indol-3-yl)methylene]-1-benzofuran-3(2H)-one;
2-[(2-bromo-1H-indol-3-yl)methylene]-4,6-dihydroxy-1-benzofuran-3(2H)-one;
2-[(2-bromo-1H-indol-3-yl)methylene]-6,7-dihydroxy-1-benzofuran-3(2H)-one;
4-hydroxy-2-[(5-methoxy-2-methyl-1H-indol-3-yl)methylene]-1-benzofuran-3(2H)-one;
2-{[1-(4-chlorobutyl)-5-methoxy-2-methyl-1H-indol-3-yl]methylene}-4,6-dihydroxy-1-benzofuran-3(2H)-one;
2-{[1-(3-chloropropyl)-5-methoxy-2-methyl-1H-indol-3-yl]methylene}-4,6-dihydroxy-1-benzofuran-3(2H)-one;
4,6-dihydroxy-2-[(5-methoxy-2-pyridin-3-yl-1H-indol-3-yl)methylene]-1-benzofuran-3(2H)-one;
2-{[1-(2-chloroethyl)-2-methyl-1H-indol-3-yl]methylene}-4,6-dihydroxy-1-benzofuran-3(2H)-one;
2-{[1-(3-chloropropyl)-2-methyl-1H-indol-3-yl]methylene}-4,6-dihydroxy-1-benzofuran-3(2H)-one;
2-{[1-(4-chlorobutyl)-2-methyl-1H-indol-3-yl]methylene}-4,6-dihydroxy-1-benzofuran-3(2H)-one;
4,6-dihydroxy-2-({5-methoxy-2-methyl-1-[3-(4-methylpiperazin-1-yl)propyl]-1H-indol-3-yl}methylene)-1-benzofuran-3(2H)-one;
4,6-dihydroxy-2-({5-methoxy-2-methyl-1-[4-(4-methylpiperazin-1-yl)butyl]-1H-indol-3-yl}methylene)-1-benzofuran-3(2H)-one;
4,6-dihydroxy-2-{[5-methoxy-2-methyl-1-(4-morpholin-4-ylbutyl)-1H-indol-3-yl]methylene}-1-benzofuran-3(2H)-one;
4,6-dihydroxy-2-[(1-{4-[4-(2-hydroxyethyl)piperazin-1-yl]butyl}-5-methoxy-2-methyl-1H-indol-3-yl)methylene]-1-benzofuran-3(2H)-one;
2-[(1-{4-[3-(dimethylamino)pyrrolidin-1-yl]butyl}-5-methoxy-2-methyl-1H-indol-3-yl)methylene]-4,6-dihydroxy-1-benzofuran-3(2H)-one;
4,6-dihydroxy-2-[(5-methoxy-2-methyl-1-{4-[4-(2-morpholin-4-ylethyl)piperazin-1-yl]butyl}-1H-indol-3-yl)methylene]-1-benzofuran-3(2H)-one;
2-({1-[4-(dimethylamino)butyl]-5-methoxy-2-methyl-1H-indol-3-yl}methylene)-4,6-dihydroxy-1-benzofuran-3(2H)-one;
4,6-dihydroxy-2-{[5-methoxy-2-methyl-1-(3-morpholin-4-ylpropyl)-1H-indol-3-yl]methylene}-1-benzofuran-3(2H)-one;
4,6-dihydroxy-2-[(1-{3-[4-(2-hydroxyethyl)piperazin-1-yl]propyl}-5-methoxy-2-methyl-1H-indol-3-yl)methylene]-1-benzofuran-3(2H)-one;
2-[(1-{3-[3-(dimethylamino)pyrrolidin-1-yl]propyl}-5-methoxy-2-methyl-1H-indol-3-yl)methylene]-4,6-dihydroxy-1-benzofuran-3(2H)-one;
4,6-dihydroxy-2-[(5-methoxy-2-methyl-1-{3-[4-(2-morpholin-4-ylethyl)piperazin-1-yl]propyl}-1H-indol-3-yl)methylene]-1-benzofuran-3(2H)-one;
2-({1-[3-(dimethylamino)propyl]-5-methoxy-2-methyl-1H-indol-3-yl}methylene)-4,6-dihydroxy-1-benzofuran-3(2H)-one;
4,6-dihydroxy-2-{[5-methoxy-2-methyl-1-(2-morpholin-4-ylethyl)-1H-indol-3-yl]methylene}-1-benzofuran-3(2H)-one;
4,6-dihydroxy-2-({5-methoxy-2-methyl-1-[2-(4-methylpiperazin-1-yl)ethyl]-1H-indol-3-yl}methylene)-1-benzofuran-3(2H)-one;
4,6-dihydroxy-2-[(1-{2-[4-(2-hydroxyethyl)piperazin-1-yl]ethyl}-5-methoxy-2-methyl-1H-indol-3-yl)methylene]-1-benzofuran-3(2H)-one;
2-({1-[2-(dimethylamino)ethyl]-5-methoxy-2-methyl-1H-indol-3-yl}methylene)-4,6-dihydroxy-1-benzofuran-3(2H)-one;
4,6-dihydroxy-2-[(1-{2-[4-(2-hydroxyethyl)piperazin-1-yl]ethyl}-5-methoxy-1H-indol-3-yl)methylene]-1-benzofuran-3(2H)-one;
4,6-dihydroxy-2-{[5-methoxy-1-(3-morpholin-4-ylpropyl)-1H-indol-3-yl]methylene}-1-benzofuran-3(2H)-one;
4,6-dihydroxy-2-[(1-{3-[4-(2-hydroxyethyl)piperazin-1-yl]propyl}-5-methoxy-1H-indol-3-yl)methylene]-1-benzofuran-3(2H)-one;
2-({1-[3-(dimethylamino)propyl]-5-methoxy-1H-indol-3-yl}methylene)-4,6-dihydroxy-1-benzofuran-3(2H)-one;
4,6-dihydroxy-2-{[5-methoxy-1-(4-morpholin-4-ylbutyl)-1H-indol-3-yl]methylene}-1-benzofuran-3(2H)-one;
2-({1-[4-(dimethylamino)butyl]-5-methoxy-1H-indol-3-yl}methylene)-4,6-dihydroxy-1-benzofuran-3(2H)-one;
4,6-dihydroxy-2-[(5-methoxy-1H-indol-3-yl)methyl]-1-benzofuran-3(2H)-one;
4,6-dihydroxy-2-[(5-methoxy-2-phenyl-1H-indol-3-yl)methyl]-1-benzofuran-3(2H)-one;
4,6-dihydroxy-2-[(5-methoxy-2-methyl-1H-indol-3-yl)methyl]-1-benzofuran-3(2H)-one;
7-hydroxy-2-[(5-methoxy-2-methyl-1H-indol-3-yl)methylene]-1-benzofuran-3(2H)-one;
4-hydroxy-2-[(5-methoxy-1,2-dimethyl-1H-indol-3-yl)methylene]-1-benzofuran-3(2H)-one;
2-{[1-(4-chlorobutyl)-5-methoxy-2-methyl-1H-indol-3-yl]methylene}-6,7-dihydroxy-1-benzofuran-3(2H)-one;
2-{[1-(4-chlorobutyl)-5-methoxy-2-methyl-1H-indol-3-yl]methylene}-4-hydroxy-1-benzofuran-3(2H)-one;
2-{[1-(3-chloropropyl)-5-methoxy-2-methyl-1H-indol-3-yl]methylene}-6,7-dihydroxy-1-benzofuran-3(2H)-one;
2-{[1-(3-chloropropyl)-5-methoxy-2-methyl-1H-indol-3-yl]methylene}-4-hydroxy-1-benzofuran-3(2H)-one;
4-hydroxy-2-({5-methoxy-2-methyl-1-[2-(4-methylpiperazin-1-yl)ethyl]-1H-indol-3-yl}methylene)-1-benzofuran-3(2H)-one;
4-hydroxy-2-{[5-methoxy-2-methyl-1-(2-morpholin-4-ylethyl)-1H-indol-3-yl]methylene}-1-benzofuran-3(2H)-one;
4-hydroxy-2-[(1-{2-[4-(2-hydroxyethyl)piperazin-1-yl]ethyl}-5-methoxy-2-methyl-1H-indol-3-yl)methylene]-1-benzofuran-3(2H)-one;
6,7-dihydroxy-2-({5-methoxy-2-methyl-1-[4-(4-methylpiperazin-1-yl)butyl]-1H-indol-3-yl}methylene)-1-benzofuran-3(2H)-one;
6,7-dihydroxy-2-{[5-methoxy-2-methyl-1-(4-morpholin-4-ylbutyl)-1H-indol-3-yl]methylene}-1-benzofuran-3(2H)-one;
6,7-dihydroxy-2-[(1-{4-[4-(2-hydroxyethyl)piperazin-1-yl]butyl}-5-methoxy-2-methyl-1H-indol-3-yl)methylene]-1-benzofuran-3(2H)-one;
4-hydroxy-2-({5-methoxy-2-methyl-1-[4-(4-methylpiperazin-1-yl)butyl]-1H-indol-3-yl}methylene)-1-benzofuran-3(2H)-one;
4-hydroxy-2-{[5-methoxy-2-methyl-1-(4-morpholin-4-ylbutyl)-1H-indol-3-yl]methylene}-1-benzofuran-3(2H)-one;
4-hydroxy-2-[(1-{4-[4-(2-hydroxyethyl)piperazin-1-yl]butyl}-5-methoxy-2-methyl-1H-indol-3-yl)methylene]-1-benzofuran-3(2H)-one;
2-[(6-bromo-1H-indol-3-yl)methylene]-4,6-dihydroxy-1-benzofuran-3(2H)-one;
6,7-dihydroxy-2-({5-methoxy-2-methyl-1-[4-(4-methylpiperazin-1-yl)butyl]-1H-indol-3-yl}methylene)-1-benzofuran-3(2H)-one;
6,7-dihydroxy-2-{[5-methoxy-2-methyl-1-(4-morpholin-4-ylbutyl)-1H-indol-3-yl]methylene}-1-benzofuran-3(2H)-one;
6,7-dihydroxy-2-[(1-{4-[4-(2-hydroxyethyl)piperazin-1-yl]butyl}-5-methoxy-2-methyl-1H-indol-3-yl)methylene]-1-benzofuran-3(2H)-one;
4,6-dihydroxy-2-{[1-(2-morpholin-4-ylethyl)-2-phenyl-1H-indol-3-yl]methylene}-1-benzofuran-3(2H)-one;
2-({1-[2-(dimethylamino)ethyl]-2-phenyl-1H-indol-3-yl}methylene)-4,6-dihydroxy-1-benzofuran-3(2H)-one;
2-[(1-benzyl-2-phenyl-1H-indol-3-yl)methylene]-4,6-dihydroxy-1-benzofuran-3(2H)-one;
4,6-dihydroxy-2-[(1-isobutyl-2-phenyl-1H-indol-3-yl)methylene]-1-benzofuran-3(2H)-one;
4,6-dihydroxy-2-{[1-(2-methoxyethyl)-2-phenyl-1H-indol-3-yl]methylene}-1-benzofuran-3(2H)-one;
2-{[1-(cyclopropylmethyl)-2-phenyl-1H-indol-3-yl]methylene}-4,6-dihydroxy-1-benzofuran-3(2H)-one;
4,6-dihydroxy-2-{[2-phenyl-1-(pyridin-3-ylmethyl)-1H-indol-3-yl]methylene}-1-benzofuran-3(2H)-one;
4,6-dihydroxy-2-{[2-phenyl-1-(pyridin-4-ylmethyl)-1H-indol-3-yl]methylene}-1-benzofuran-3(2H)-one;
4-{3-[(4,6-dihydroxy-3-oxo-1-benzofuran-2(3H)-ylidene)methyl]-2-phenyl-1H-indol-1-yl}butanenitrile;
2-({1-[3-(dimethylamino)propyl]-2-phenyl-1H-indol-3-yl}methylene)-4,6-dihydroxy-1-benzofuran-3(2H)-one;
4,6-dihydroxy-2-{[2-phenyl-1-(2-pyrrolidin-1-ylethyl)-1H-indol-3-yl]methylene}-1-benzofuran-3(2H)-one;
4,6-dihydroxy-2-{[2-phenyl-1-(2-piperidin-4-ylethyl)-1H-indol-3-yl]methylene}-1-benzofuran-3(2H)-one;
4,6-dihydroxy-2-({1-[2-(4-methylpiperazin-1-yl)ethyl]-2-phenyl-1H-indol-3-yl}methylene)-1-benzofuran-3(2H)-one;
4,6-dihydroxy-2-{[2-phenyl-1-(2-piperazin-1-ylethyl)-1H-indol-3-yl]methylene}-1-benzofuran-3(2H)-one;
2-{3-[(4,6-dihydroxy-3-oxo-1-benzofuran-2(3H)-ylidene)methyl]-2-phenyl-1H-indol-1-yl}acetamide;
4,6-dihydroxy-2-[(2-methyl-5-nitro-1H-indol-3-yl)methylene]-1-benzofuran-3(2H)-one;
4,6-dihydroxy-2-[(5-hydroxy-1H-indol-3-yl)methylene]-1-benzofuran-3(2H)-one;
3-[(4,6-dihydroxy-3-oxo-1-benzofuran-2(3H)-ylidene)methyl]-1H-indole-5-carboxylic acid;
methyl 3-[(4,6-dihydroxy-3-oxo-1-benzofuran-2(3H)-ylidene)methyl]-1H-indole-5-carboxylate;
3-[(4,6-dihydroxy-3-oxo-1-benzofuran-2(3H)-ylidene)methyl]-1H-indole-6-carbonitrile;
2-(5H-[1,3]dioxolo[4,5-f]indol-7-ylmethylene)-4,6-dihydroxy-1-benzofuran-3(2H)-one;
4,6-dihydroxy-2-{[6-(methylsulfonyl)-1H-indol-3-yl]methylene}-1-benzofuran-3(2H)-one;
4,6-dihydroxy-2-[(5-methyl-1H-indol-3-yl)methylene]-1-benzofuran-3(2H)-one;
2-[(4-chloro-1H-indol-3-yl)methylene]-4,6-dihydroxy-1-benzofuran-3(2H)-one;
2-[(6-chloro-1H-indol-3-yl)methylene]-4,6-dihydroxy-1-benzofuran-3(2H)-one;
2-[(7-chloro-1H-indol-3-yl)methylene]-4,6-dihydroxy-1-benzofuran-3(2H)-one;
2-[(4-bromo-1H-indol-3-yl)methylene]-4,6-dihydroxy-1-benzofuran-3(2H)-one;
2-[(5-fluoro-1H-indol-3-yl)methylene]-4,6-dihydroxy-1-benzofuran-3(2H)-one;
2-[(6-fluoro-1H-indol-3-yl)methylene]-4,6-dihydroxy-1-benzofuran-3(2H)-one;
4,6-dihydroxy-2-[(5-iodo-1H-indol-3-yl)methylene]-1-benzofuran-3(2H)-one;
4,6-dihydroxy-2-[(5-nitro-1H-indol-3-yl)methylene]-1-benzofuran-3(2H)-one;
4,6-dihydroxy-2-[(6-nitro-1H-indol-3-yl)methylene]-1-benzofuran-3(2H)-one;
4,6-dihydroxy-2-[(7-nitro-1H-indol-3-yl)methylene]-1-benzofuran-3(2H)-one;
2-[(5,6-dimethoxy-1H-indol-3-yl)methylene]-4,6-dihydroxy-1-benzofuran-3(2H)-one;
3-[(4,6-dihydroxy-3-oxo-1-benzofuran-2(3H)-ylidene)methyl]-1H-indole-5-carbonitrile;
N-{3-[(4,6-dihydroxy-3-oxo-1-benzofuran-2(3H)-ylidene)methyl]-1H-indol-5-yl}-2-furamide;
4,6-dihydroxy-2-[(5-methoxy-2,6-dimethyl-1H-indol-3-yl)methylene]-1-benzofuran-3(2H)-one;
4,6-dihydroxy-2-[(5-methoxy-1,2,6-trimethyl-1H-indol-3-yl)methylene]-1-benzofuran-3(2H)-one;
4,6-dihydroxy-2-[(6-methoxy-1H-indol-3-yl)methylene]-1-benzofuran-3(2H)-one;
2-[(7-ethyl-1H-indol-3-yl)methylene]-4,6-dihydroxy-1-benzofuran-3(2H)-one;
3-[(4,6-dihydroxy-3-oxo-1-benzofuran-2(3H)-ylidene)methyl]-1-methyl-1H-indole-4-carbonitrile;
4,6-dihydroxy-2-[(1-methyl-2-pyridin-3-yl-1H-indol-3-yl)methylene]-1-benzofuran-3(2H)-one;
4,6-dihydroxy-2-[(1-methyl-2-pyridin-4-yl-1H-indol-3-yl)methylene]-1-benzofuran-3(2H)-one;
2-{[2-(3,5-dimethylisoxazol-4-yl)-1-methyl-1H-indol-3-yl]methylene}-4,6-dihydroxy-1-benzofuran-3(2H)-one;
4,6-dihydroxy-2-{[2-(3-hydroxyphenyl)-1-methyl-1H-indol-3-yl]methylene}-1-benzofuran-3(2H)-one;
4,6-dihydroxy-2-{[2-(4-hydroxyphenyl)-1-methyl-1H-indol-3-yl]methylene}-1-benzofuran-3(2H)-one;
4,6-dihydroxy-2-{[1-methyl-2-(3-thienyl)-1H-indol-3-yl]methylene}-1-benzofuran-3(2H)-one;
4,6-dihydroxy-2-{[2-(4-methoxyphenyl)-1-methyl-1H-indol-3-yl]methylene}-1-benzofuran-3(2H)-one;
4,6-dihydroxy-2-{[2-(3-methoxyphenyl)-1-methyl-1H-indol-3-yl]methylene}-1-benzofuran-3(2H)-one;
2-{[2-(3-fluorophenyl)-1-methyl-1H-indol-3-yl]methylene}-4,6-dihydroxy-1-benzofuran-3(2H)-one;
2-({2-[4-(dimethylamino)phenyl]-1-methyl-1H-indol-3-yl}methylene)-4,6-dihydroxy-1-benzofuran-3(2H)-one;
2-{[2-(3-chloro-4-fluorophenyl)-1-methyl-1H-indol-3-yl]methylene}-4,6-dihydroxy-1-benzofuran-3(2H)-one;
2-{[2-(4-fluorophenyl)-1-methyl-1H-indol-3-yl]methylene}-4,6-dihydroxy-1-benzofuran-3(2H)-one;
2-{[2-(4-chlorophenyl)-1-methyl-1H-indol-3-yl]methylene}-4,6-dihydroxy-1-benzofuran-3(2H)-one;
4,6-dihydroxy-2-[(2-pyridin-3-yl-1H-indol-3-yl)methylene]-1-benzofuran-3(2H)-one;
4,6-dihydroxy-2-[(2-pyridin-4-yl-1H-indol-3-yl)methylene]-1-benzofuran-3(2H)-one;
2-{[2-(3,5-dimethylisoxazol-4-yl)-1H-indol-3-yl]methylene}-4,6-dihydroxy-1-benzofuran-3(2H)-one;
4,6-dihydroxy-2-{[2-(3-hydroxyphenyl)-1H-indol-3-yl]methylene}-1-benzofuran-3(2H)-one;
4,6-dihydroxy-2-{[2-(4-hydroxyphenyl)-1H-indol-3-yl]methylene}-1-benzofuran-3(2H)-one;
4,6-dihydroxy-2-{[2-(3-thienyl)-1H-indol-3-yl]methylene}-1-benzofuran-3(2H)-one;
4,6-dihydroxy-2-{[2-(4-methoxyphenyl)-1H-indol-3-yl]methylene}-1-benzofuran-3(2H)-one;
4,6-dihydroxy-2-{[2-(3-methoxyphenyl)-1H-indol-3-yl]methylene}-1-benzofuran-3(2H)-one;
2-{[2-(3-fluorophenyl)-1H-indol-3-yl]methylene}-4,6-dihydroxy-1-benzofuran-3(2H)-one;
2-({2-[4-(dimethylamino)phenyl]-1H-indol-3-yl}methylene)-4,6-dihydroxy-1-benzofuran-3(2H)-one;
2-{[2-(3-chloro-4-fluorophenyl)-1H-indol-3-yl]methylene}-4,6-dihydroxy-1-benzofuran-3(2H)-one;
2-[(1-ethyl-2-phenyl-1H-indol-3-yl)methylene]-4,6-dihydroxy-1-benzofuran-3(2H)-one;
4,6-dihydroxy-2-[(2-phenyl-1-propyl-1H-indol-3-yl)methylene]-1-benzofuran-3(2H)-one;
2-[(5-chloro-2-methyl-1H-indol-3-yl)methylene]-4,6-dihydroxy-1-benzofuran-3(2H)-one;
2-[(7-bromo-2-methyl-1H-indol-3-yl)methylene]-4,6-dihydroxy-1-benzofuran-3(2H)-one;
2-[(5-fluoro-2-methyl-1H-indol-3-yl)methylene]-4,6-dihydroxy-1-benzofuran-3(2H)-one;
4,6-dihydroxy-2-[(5-methoxy-4-methyl-1H-indol-3-yl)methylene]-1-benzofuran-3(2H)-one;
3-[(4,6-dihydroxy-3-oxo-1-benzofuran-2(3H)-ylidene)methyl]-1H-indole-4-carbonitrile;
4,6-dihydroxy-2-{[6-(trifluoromethyl)-1H-indol-3-yl]methylene}-1-benzofuran-3(2H)-one;
methyl 3-[(4,6-dihydroxy-3-oxo-1-benzofuran-2(3H)-ylidene)methyl]-1H-indole-4-carboxylate;
4,6-dihydroxy-2-[(1-methyl-2-pyridin-2-yl-1H-indol-3-yl)methylene]-1-benzofuran-3(2H)-one;
5-{3-[(4,6-dihydroxy-3-oxo-1-benzofuran-2(3H)-ylidene)methyl]-2-phenyl-1H-indol-1-yl}pentanenitrile;
6-{3-[(4,6-dihydroxy-3-oxo-1-benzofuran-2(3H)-ylidene)methyl]-2-phenyl-1H-indol-1-yl}hexanenitrile;
4-{3-[(4,6-dihydroxy-3-oxo-1-benzofuran-2(3H)-ylidene)methyl]-2-pyridin-3-yl-1H-indol-1-yl}butanenitrile;
2-[(5-fluoro-1-methyl-1H-indol-3-yl)methylene]-4,6-dihydroxy-1-benzofuran-3(2H)-one;
4,6-dihydroxy-2-[(1-methyl-5-nitro-1H-indol-3-yl)methylene]-1-benzofuran-3(2H)-one;
4,6-dihydroxy-2-[(1-methyl-7-nitro-1H-indol-3-yl)methylene]-1-benzofuran-3(2H)-one;
4-{4-bromo-3-[(4,6-dihydroxy-3-oxo-1-benzofuran-2(3H)-ylidene)methyl]-1H-indol-1-yl}butanenitrile;
4-{3-[(4,6-dihydroxy-3-oxo-1-benzofuran-2(3H)-ylidene)methyl]-5-fluoro-1H-indol-1-yl}butanenitrile;
4-{3-[(4,6-dihydroxy-3-oxo-1-benzofuran-2(3H)-ylidene)methyl]-7-ethyl-1H-indol-1-yl}butanenitrile;
2-[(5-chloro-1,2-dimethyl-1H-indol-3-yl)methylene]-4,6-dihydroxy-1-benzofuran-3(2H)-one;
2-[(7-bromo-1,2-dimethyl-1H-indol-3-yl)methylene]-4,6-dihydroxy-1-benzofuran-3(2H)-one;
2-[(5-fluoro-1,2-dimethyl-1H-indol-3-yl)methylene]-4,6-dihydroxy-1-benzofuran-3(2H)-one;
4,6-dihydroxy-2-[(5-methoxy-1,4-dimethyl-1H-indol-3-yl)methylene]-1-benzofuran-3(2H)-one;
4,6-dihydroxy-2-{[1-methyl-6-(trifluoromethyl)-1H-indol-3-yl]methylene}-1-benzofuran-3(2H)-one;
4-{5-chloro-3-[(4,6-dihydroxy-3-oxo-1-benzofuran-2(3H)-ylidene)methyl]-2-methyl-1H-indol-1-yl}butanenitrile;
4-{3-[(4,6-dihydroxy-3-oxo-1-benzofuran-2(3H)-ylidene)methyl]-5-methoxy-4-methyl-1H-indol-1-yl}butanenitrile;
4-{3-[(4,6-dihydroxy-3-oxo-1-benzofuran-2(3H)-ylidene)methyl]-2-methyl-1H-indol-1-yl}butanenitrile;
4-{3-[(4,6-dihydroxy-3-oxo-1-benzofuran-2(3H)-ylidene)methyl]-1H-indol-1-yl}butanenitrile;
2-{[7-(benzyloxy)-1H-indol-3-yl]methylene}-4,6-dihydroxy-1-benzofuran-3(2H)-one;
2-{[4-(benzyloxy)-1H-indol-3-yl]methylene}-4,6-dihydroxy-1-benzofuran-3(2H)-one;
2-[(7-bromo-1H-indol-3-yl)methylene]-4,6-dihydroxy-1-benzofuran-3(2H)-one;
methyl 3-[(4,6-dihydroxy-3-oxo-1-benzofuran-2(3H)-ylidene)methyl]-1H-indole-7-carboxylate;
4,6-dihydroxy-2-[(7-hydroxy-1H-indol-3-yl)methylene]-1-benzofuran-3(2H)-one;
2-[(5-bromo-1-methyl-1H-indol-3-yl)methylene]-4,6-dihydroxy-1-benzofuran-3(2H)-one;
2-[(7-bromo-1-methyl-1H-indol-3-yl)methylene]-4,6-dihydroxy-1-benzofuran-3(2H)-one;
methyl 3-[(4,6-dihydroxy-3-oxo-1-benzofuran-2(3H)-ylidene)methyl]-1-methyl-1H-indole-7-carboxylate;
4,6-dihydroxy-2-[(7-methoxy-1-methyl-1H-indol-3-yl)methylene]-1-benzofuran-3(2H)-one;
2-[(4-chloro-1-methyl-1H-indol-3-yl)methylene]-4,6-dihydroxy-1-benzofuran-3(2H)-one;
2-[(4-bromo-1-methyl-1H-indol-3-yl)methylene]-4,6-dihydroxy-1-benzofuran-3(2H)-one;
4,6-dihydroxy-2-[(4-hydroxy-1-methyl-1H-indol-3-yl)methylene]-1-benzofuran-3(2H)-one;
4,6-dihydroxy-2-[(4-methoxy-1-methyl-1H-indol-3-yl)methylene]-1-benzofuran-3(2H)-one;
2-{[4-(benzyloxy)-1-methyl-1H-indol-3-yl]methylene}-4,6-dihydroxy-1-benzofuran-3(2H)-one;
4-{3-[(4,6-dihydroxy-3-oxo-1-benzofuran-2(3H)-ylidene)methyl]-4-hydroxy-1H-indol-1-yl}butanenitrile;
4,6-dihydroxy-2-[(2-{4-[2-(2-methoxyethoxy)ethoxy]phenyl}-1-methyl-1H-indol-3-;
2-({2-[4-(benzyloxy)phenyl]-1-methyl-1H-indol-3-yl}methylene)-4,6-dihydroxy-1-benzofuran-3(2H)-one;
4,6-dihydroxy-2-{[2-(4-isopropoxyphenyl)-1-methyl-1H-indol-3-yl]methylene}-1-benzofuran-3(2H)-one;
3-[(4,6-dihydroxy-3-oxo-1-benzofuran-2(3H)-ylidene)methyl]-1-(2-morpholin-4-ylethyl)-1H-indole-4-carbonitrile;
3-[(4,6-dihydroxy-3-oxo-1-benzofuran-2(3H)-ylidene)methyl]-1-(pyridin-4-ylmethyl)-1H-indole-4-carbonitrile;
1-(3-cyanopropyl)-3-[(4,6-dihydroxy-3-oxo-1-benzofuran-2(3H)-ylidene)methyl]-1H-indole-4-carbonitrile;
3-[(4,6-dihydroxy-3-oxo-1-benzofuran-2(3H)-ylidene)methyl]-1-[2-(dimethylamino)ethyl]-1H-indole-4-carbonitrile;
3-[(4,6-dihydroxy-3-oxo-1-benzofuran-2(3H)-ylidene)methyl]-1-(2-methoxyethyl)-1H-indole-4-carbonitrile;
methyl 3-[(4,6-dihydroxy-3-oxo-1-benzofuran-2(3H)-ylidene)methyl]-1-methyl-1H-indole-4-carboxylate;
4,6-dihydroxy-2-[(1-methyl-4-phenyl-1H-indol-3-yl)methylene]-1-benzofuran-3(2H)-one;
4,6-dihydroxy-2-[(4-phenyl-1H-indol-3-yl)methylene]-1-benzofuran-3(2H)-one;
(2Z)-6,7-dihydroxy-2-[(2-phenyl-1H-indol-3-yl)methylene]-1-benzofuran-3(2H)-one;
(2Z)-2-(1H-indol-3-ylmethylene)-1-benzothiophen-3(2H)-one;
(2Z)-2-[(2-methyl-1H-indol-3-yl)methylene]-1-benzothiophen-3(2H)-one;
(2Z)-2-[(2-phenyl-1H-indol-3-yl)methylene]-1-benzothiophen-3(2H)-one;
(2Z)-2-[(1-methyl-2-phenyl-1H-indol-3-yl)methylene]-1-benzothiophen-3(2H)-one;
(2Z)-2-{[2-(2-naphthyl)-1H-indol-3-yl]methylene}-1-benzothiophen-3(2H)-one;
(2Z)-2-{[2-(4-fluorophenyl)-1H-indol-3-yl]methylene}-1-benzothiophen-3(2H)-one;
(2Z)-2-[(6-methyl-1H-indol-3-yl)methylene]-1-benzothiophen-3(2H)-one;
(2Z)-2-[(7-methyl-1H-indol-3-yl)methylene]-1-benzothiophen-3(2H)-one;
(2Z)-5-chloro-2-(1H-indol-3-ylmethylene)-1-benzothiophen-3(2H)-one;
(2Z)-5-chloro-2-{[2-(4-chlorophenyl)-1H-indol-3-yl]methylene}-1-benzothiophen-3(2H)-one;
(2Z)-5-chloro-2-{[2-(2-naphthyl)-1H-indol-3-yl]methylene}-1-benzothiophen-3(2H)-one;
(2Z)-5-chloro-2-{[2-(4-fluorophenyl)-1H-indol-3-yl]methylene}-1-benzothiophen-3(2H)-one;
(2Z)-2-[(1-benzyl-1H-indol-3-yl)methylene]-5-chloro-1-benzothiophen-3(2H)-one;
(2Z)-2-{[5-(benzyloxy)-1H-indol-3-yl]methylene}-5-chloro-1-benzothiophen-3(2H)-one;
(2Z)-2-(1H-indol-3-ylmethylene)-5-methyl-1-benzothiophen-3(2H)-one;
(2Z)-5-methyl-2-[(2-phenyl-1H-indol-3-yl)methylene]-1-benzothiophen-3(2H)-one;
(2Z)-2-{[2-(4-chlorophenyl)-1H-indol-3-yl]methylene}-5-methyl-1-benzothiophen-3(2H)-one;
(2Z)-5-methyl-2-[(6-methyl-1H-indol-3-yl)methylene]-1-benzothiophen-3(2H)-one;
(2Z)-5-methyl-2-[(7-methyl-1H-indol-3-yl)methylene]-1-benzothiophen-3(2H)-one;
(2Z)-2-[(5-bromo-1H-indol-3-yl)methylene]-5-methyl-1-benzothiophen-3(2H)-one;
(2Z)-2-{[5-(benzyloxy)-1H-indol-3-yl]methylene}-5-methyl-1-benzothiophen-3(2H)-one;
5-methyl-2-{[2-(2-naphthyl)-1H-indol-3-yl]methylene}-1-benzothiophen-3(2H)-one;
2-{[2-(4-fluorophenyl)-1H-indol-3-yl]methylene}-5-methyl-1-benzothiophen-3(2H)-one;
5-methyl-2-[(2-methyl-5-nitro-1H-indol-3-yl)methylene]-1-benzothiophen-3(2H)-one;
5-methyl-2-[(1-methyl-1H-indol-3-yl)methylene]-1-benzothiophen-3(2H)-one;
(2Z)-2-({4-[4′-(aminomethyl)biphenyl-4-yl]-1-methyl-1H-indol-3-yl}methylene)-4,6-dihydroxy-1-benzofuran-3(2H)-one;
(2Z)-2-[(4-{4′-[(dimethylamino)methyl]biphenyl-4-yl}-1-methyl-1H-indol-3-yl)methylene]-4,6-dihydroxy-1-benzofuran-3(2H)-one;
(2Z)-6-hydroxy-2-({5-methoxy-2-methyl-1-[2-(4-methylpiperazin-1-yl)ethyl]-1H-indol-3-yl}methylene)-1-benzofuran-3(2H)-one;
(2Z)-2-({2-cyclopropyl-5-methoxy-1-[2-(4-methylpiperazin-1-yl)ethyl]-1H-indol-3-yl}methylene)-4,6-dihydroxy-1-benzofuran-3(2H)-one;
(2Z)-4-hydroxy-2-[(5-methoxy-2-phenyl-1H-indol-3-yl)methylene]-1-benzofuran-3(2H)-one;
(2Z)-2-({1-[3-(dimethylamino)propyl]-5-methoxy-2-methyl-1H-indol-3-yl}methylene)-4,6-dihydroxy-1-benzofuran-3(2H)-one;
3-[(Z)-(4,6-dihydroxy-3-oxo-1-benzofuran-2(3H)-ylidene)methyl]-1-[2-(3-hydroxypyrrolidin-1-yl)ethyl]-5-methoxy-N,N-dimethyl-1H-indole-2-carboxamide;
(2Z)-2-({1-[3-(dimethylamino)propyl]-5-methoxy-2-methyl-1H-indol-3-yl}methylene)-6-hydroxy-1-benzofuran-3(2H)-one;
(2Z)-4,6-dihydroxy-2-({5-methoxy-1-[2-(4-methylpiperazin-1-yl)ethyl]-1H-indol-3-yl}methylene)-1-benzofuran-3(2H)-one;
(2Z)-4,6-dihydroxy-2-({5-methoxy-2-methyl-1-[2-(4-methylpiperazin-1-yl)ethyl]-1H-indol-3-yl}methylene)-1-benzofuran-3(2H)-one;
(2Z)-6-hydroxy-2-[(5-methoxy-2-phenyl-1H-indol-3-yl)methylene]-1-benzofuran-3(2H)-one;
3-[(Z)-(4,6-dihydroxy-3-oxo-1-benzofuran-2(3H)-ylidene)methyl]-1-[2-(4-hydroxypiperidin-1-yl)ethyl]-5-methoxy-N,N-dimethyl-1H-indole-2-carboxamide;
(2Z)-4,6-dihydroxy-2-({1-[2-(1H-imidazol-1-yl)ethyl]-5-methoxy-1H-indol-3-yl}methylene)-1-benzofuran-3(2H)-one;
(2Z)-2-{[2-cyclopropyl-1-(2-hydroxyethyl)-5-methoxy-1H-indol-3-yl]methylene}-4,6-dihydroxy-1-benzofuran-3(2H)-one;
3-[(Z)-(4,6-dihydroxy-3-oxo-1-benzofuran-2(3H)-ylidene)methyl]-1-[2-(1H-imidazol-1-yl)ethyl]-5-methoxy-N,N-dimethyl-1H-indole-2-carboxamide;
(2Z)-2-({2-cyclopropyl-1-[2-(4-hydroxypiperidin-1-yl)ethyl]-5-methoxy-1H-indol-3-yl}methylene)-4,6-dihydroxy-1-benzofuran-3(2H)-one;
3-[(Z)-(4,6-dihydroxy-3-oxo-1-benzofuran-2(3H)-ylidene)methyl]-5-methoxy-N,N-dimethyl-1-[2-(4-methylpiperazin-1-yl)ethyl]-1H-indole-2-carboxamide;
(2Z)-4,6-dihydroxy-2-{[1-(2-hydroxyethyl)-5-methoxy-1H-indol-3-yl]methylene}-1-benzofuran-3(2H)-one;
2-{3-[(Z)-(4,6-dihydroxy-3-oxo-1-benzofuran-2(3H)-ylidene)methyl]-5-methoxy-1H-indol-1-yl}-N,N-dimethylacetamide;
(2Z)-4,6-dihydroxy-2-({1-[2-(4-hydroxypiperidin-1-yl)ethyl]-5-methoxy-1H-indol-3-yl}methylene)-1-benzofuran-3(2H)-one;
(2Z)-4,6-dihydroxy-2-({1-[2-(4-hydroxypiperidin-1-yl)ethyl]-5-methoxy-2-methyl-1H-indol-3-yl}methylene)-1-benzofuran-3(2H)-one;
(2Z)-4,6-dihydroxy-2-{[1-(2-hydroxyethyl)-5-methoxy-2-methyl-1H-indol-3-yl]methylene}-1-benzofuran-3(2H)-one;
2-{3-[(Z)-(4,6-dihydroxy-3-oxo-1-benzofuran-2(3H)-ylidene)methyl]-5-methoxy-2-methyl-1H-indol-1-yl}-N,N-dimethylacetamide;
(2Z)-2-({1-[3-(dimethylamino)propyl]-5-methoxy-1H-indol-3-yl}methylene)-4,6-dihydroxy-1-benzofuran-3(2H)-one;
(2Z)-2-({1-[3-(dimethylamino)propyl]-5-methoxy-1H-indol-3-yl}methylene)-6-hydroxy-1-benzofuran-3(2H)-one;
3-[(Z)-(4,6-dihydroxy-3-oxo-1-benzofuran-2(3H)-ylidene)methyl]-1-[2-(dimethylamino)-2-oxoethyl]-5-methoxy-N,N-dimethyl-1H-indole-2-carboxamide;
(2Z)-4,6-dihydroxy-2-({1-[2-(3-hydroxypyrrolidin-1-yl)ethyl]-5-methoxy-1H-indol-3-yl}methylene)-1-benzofuran-3(2H)-one;
(2Z)-4,6-dihydroxy-2-({1-[2-(3-hydroxypyrrolidin-1-yl)ethyl]-5-methoxy-2-methyl-1H-indol-3-yl}methylene)-1-benzofuran-3(2H)-one;
(2Z)-4,6-dihydroxy-2-({1-[2-(1H-imidazol-1-yl)ethyl]-5-methoxy-2-methyl-1H-indol-3-yl}methylene)-1-benzofuran-3(2H)-one;
2-{3-[(Z)-(4,6-dihydroxy-3-oxo-1-benzofuran-2(3H)-ylidene)methyl]-5-methoxy-2-(1-methyl-1H-pyrazol-4-yl)-1H-indol-1-yl}-N,N-dimethylacetamide;
(2Z)-4,6-dihydroxy-2-{[5-methoxy-2-(1-methyl-1H-pyrazol-4-yl)-1H-indol-3-yl]methylene}-1-benzofuran-3(2H)-one;
(2Z)-4,6-dihydroxy-2-({5-methoxy-2-[(4-methylpiperazin-1-yl)carbonyl]-1H-indol-3-yl}methylene)-1-benzofuran-3(2H)-one;
Z)-4,6-dihydroxy-2-{[1-(2-hydroxyethyl)-5-methoxy-2-(trifluoromethyl)-1H-indol-3-yl]methylene}-1-benzofuran-3(2H)-one;
2-{3-[(Z)-(4,6-dihydroxy-3-oxo-1-benzofuran-2(3H)-ylidene)methyl]-5-methoxy-2-(trifluoromethyl)-1H-indol-1-yl}-N,N-dimethylacetamide;
(2Z)-2-({2-cyclopropyl-1-[2-(1H-imidazol-1-yl)ethyl]-5-methoxy-1H-indol-3-yl}methylene)-4,6-dihydroxy-1-benzofuran-3(2H)-one;
3-[(Z)-(6-hydroxy-3-oxo-1-benzofuran-2(3H)-ylidene)methyl]-5-methoxy-N,N-dimethyl-1-[2-(4-methylpiperazin-1-yl)ethyl]-1H-indole-2-carboxamide;
(2Z)-4,6-dihydroxy-2-({5-methoxy-1-[2-(4-methylpiperazin-1-yl)ethyl]-2-(trifluoromethyl)-1H-indol-3-yl}methylene)-1-benzofuran-3(2H)-one;
(2Z)-4,6-dihydroxy-2-({5-methoxy-1-[2-(1H-pyrazol-1-yl)ethyl]-2-(trifluoromethyl)-1H-indol-3-yl}methylene)-1-benzofuran-3(2H)-one;
(2Z)-6-hydroxy-2-({5-methoxy-2-methyl-1-[2-(4-methylpiperazin-1-yl)ethyl]-1H-indol-3-yl}methylene)-4-methyl-1-benzofuran-3(2H)-one;
3-[(Z)-(4,6-dihydroxy-3-oxo-1-benzofuran-2(3H)-ylidene)methyl]-5-methoxy-1H-indole-2-carboxylic acid;
(2Z)-2-({2-cyclopropyl-5-methoxy-1-[2-(1H-pyrazol-1-yl)ethyl]-1H-indol-3-yl}methylene)-4,6-dihydroxy-1-benzofuran-3(2H)-one;
(2Z)-4,6-dihydroxy-2-({5-methoxy-1-[2-(4-methylpiperazin-1-yl)ethyl]-2-(1-methyl-1H-pyrazol-4-yl)-1H-indol-3-yl}methylene)-1-benzofuran-3(2H)-one;
(2Z)-2-({2-cyclopropyl-1-[2-(3-hydroxypyrrolidin-1-yl)ethyl]-5-methoxy-1H-indol-3-yl}methylene)-4,6-dihydroxy-1-benzofuran-3(2H)-one;
3-[(Z)-(4,6-dihydroxy-3-oxo-1-benzofuran-2(3H)-ylidene)methyl]-1-[3-(dimethylamino)propyl]-5-methoxy-N,N-dimethyl-1H-indole-2-carboxamide;
(2Z)-2-({2-(3,5-dimethylisoxazol-4-yl)-5-methoxy-1-[2-(4-methylpiperazin-1-yl)ethyl]-1H-indol-3-yl}methylene)-4,6-dihydroxy-1-benzofuran-3(2H)-one;
1-[3-(dimethylamino)propyl]-3-[(Z)-(6-hydroxy-3-oxo-1-benzofuran-2(3H)-ylidene)methyl]-5-methoxy-N,N-dimethyl-1H-indole-2-carboxamide;
(2Z)-4,6-dihydroxy-2-({5-methoxy-2-[(4-methylpiperazin-1-yl)methyl]-1H-indol-3-yl}methylene)-1-benzofuran-3(2H)-one;
(2Z)-6-hydroxy-2-({5-methoxy-2-methyl-1-[2-(4-methylpiperazin-1-yl)ethyl]-1H-indol-3-yl}methylene)-7-methyl-1-benzofuran-3(2H)-one;
3-[(Z)-(4,6-dihydroxy-3-oxo-1-benzofuran-2(3H)-ylidene)methyl]-1-(2-hydroxyethyl)-5-methoxy-N,N-dimethyl-1H-indole-2-carboxamide;
Z)-5-bromo-6-hydroxy-2-({5-methoxy-2-methyl-1-[2-(4-methylpiperazin-1-yl)ethyl]-1H-indol-3-yl}methylene)-1-benzofuran-3(2H)-one;
(2Z)-5-bromo-2-({1-[3-(dimethylamino)propyl]-5-methoxy-2-methyl-1H-indol-3-yl}methylene)-6-hydroxy-1-benzofuran-3(2H)-one;
(2Z)-5-fluoro-6-hydroxy-2-({5-methoxy-2-methyl-1-[2-(4-methylpiperazin-1-yl)ethyl]-1H-indol-3-yl}methylene)-1-benzofuran-3(2H)-one;
(2Z)-2-({1-[3-(dimethylamino)propyl]-5-methoxy-2-methyl-1H-indol-3-yl}methylene)-5-fluoro-6-hydroxy-1-benzofuran-3(2H)-one;
(2Z)-2-({2-[(dimethylamino)methyl]-5-methoxy-1H-indol-3-yl}methylene)-4,6-dihydroxy-1-benzofuran-3(2H)-one;
(2Z)-4,6-dihydroxy-2-[(5-methoxy-2-pyrimidin-5-yl-1H-indol-3-yl)methylene]-1-benzofuran-3(2H)-one;
(2Z)-2-({2-cyclopropyl-5-methoxy-1-[2-(1H-pyrazol-1-yl)ethyl]-1H-indol-3-yl}methylene)-6-hydroxy-1-benzofuran-3(2H)-one;
(2Z)-6-hydroxy-2-{[5-methoxy-2-methyl-1-(3-pyrrolidin-1-ylpropyl)-1H-indol-3-yl]methylene}-1-benzofuran-3(2H)-one;
(2Z)-6-hydroxy-2-{[5-methoxy-2-methyl-1-(3-piperidin-1-ylpropyl)-1H-indol-3-yl]methylene}-1-benzofuran-3(2H)-one;
(2Z)-2-{[1-(3-azepan-1-ylpropyl)-5-methoxy-2-methyl-1H-indol-3-yl]methylene}-6-hydroxy-1-benzofuran-3(2H)-one;
(2Z)-4-fluoro-6-hydroxy-2-({5-methoxy-2-methyl-1-[2-(4-methylpiperazin-1-yl)ethyl]-1H-indol-3-yl}methylene)-1-benzofuran-3(2H)-one;
(2Z)-2-{[2-(3,5-dimethylisoxazol-4-yl)-5-methoxy-1H-indol-3-yl]methylene}-4,6-dihydroxy-1-benzofuran-3(2H)-one;
(2Z)-7-chloro-2-({1-[3-(dimethylamino)propyl]-5-methoxy-2-methyl-1H-indol-3-yl}methylene)-6-hydroxy-1-benzofuran-3(2H)-one;
(2Z)-2-({2-cyclopropyl-5-methoxy-1-[2-(4-methylpiperazin-1-yl)ethyl]-1H-indol-3-yl}methylene)-6-hydroxy-1-benzofuran-3(2H)-one;
(2Z)-2-{[4-(4-fluorophenyl)-1H-indol-3-yl]methylene}-4,6-dihydroxy-1-benzofuran-3(2H)-one;
(2Z)-2-{[4-(4-fluorophenyl)-1-methyl-1H-indol-3-yl]methylene}-4,6-dihydroxy-1-benzofuran-3(2H)-one;
(2Z)-2-{[4-(3-fluorophenyl)-1-methyl-1H-indol-3-yl]methylene}-4,6-dihydroxy-1-benzofuran-3(2H)-one;
(2Z)-2-{[4-(2-fluorophenyl)-1-methyl-1H-indol-3-yl]methylene}-4,6-dihydroxy-1-benzofuran-3(2H)-one;
3-{3-[(Z)-(4,6-dihydroxy-3-oxo-1-benzofuran-2(3H)-ylidene)methyl]-1-methyl-1H-indol-4-yl}benzonitrile;
4-{3-[(Z)-(4,6-dihydroxy-3-oxo-1-benzofuran-2(3H)-ylidene)methyl]-1-methyl-1H-indol-4-yl}benzonitrile;
(2Z)-4,6-dihydroxy-2-{[4-(6-methoxypyridin-3-yl)-1-methyl-1H-indol-3-yl]methylene}-1-benzofuran-3(2H)-one;
(2Z)-2-{[4-(4-aminophenyl)-1-methyl-1H-indol-3-yl]methylene}-4,6-dihydroxy-1-benzofuran-3(2H)-one;
(2Z)-4,6-dihydroxy-2-{[4-(4-methoxyphenyl)-1H-indol-3-yl]methylene}-1-benzofuran-3(2H)-one;
(2Z)-2-{[4-(4-acetylphenyl)-1-methyl-1H-indol-3-yl]methylene}-4,6-dihydroxy-1-benzofuran-3(2H)-one;
(2Z)-4,6-dihydroxy-2-[(1-methyl-4-pyridin-4-yl-1H-indol-3-yl)methylene]-1-benzofuran-3(2H)-one;
(2Z)-4,6-dihydroxy-2-{[1-methyl-4-(3-thienyl)-1H-indol-3-yl]methylene}-1-benzofuran-3(2H)-one;
(2Z)-4,6-dihydroxy-2-[(1-methyl-4-pyridin-3-yl-1H-indol-3-yl)methylene]-1-benzofuran-3(2H)-one;
(2Z)-4,6-dihydroxy-2-{[4-(4-methoxyphenyl)-1-methyl-1H-indol-3-yl]methylene}-1-benzofuran-3(2H)-one;
4-{3-[(Z)-(4,6-dihydroxy-3-oxo-1-benzofuran-2(3H)-ylidene)methyl]-1-methyl-1H-indol-4-yl}benzamide;
(2Z)-2-{[4-(3-furyl)-1-methyl-1H-indol-3-yl]methylene}-4,6-dihydroxy-1-benzofuran-3(2H)-one;
(2Z)-4,6-dihydroxy-2-{[4-(4-hydroxyphenyl)-1-methyl-1H-indol-3-yl]methylene}-1-benzofuran-3(2H)-one;
(2Z)-2-{[4-(6-aminopyridin-3-yl)-1-methyl-1H-indol-3-yl]methylene}-4,6-dihydroxy-1-benzofuran-3(2H)-one;
(2Z)-4,6-dihydroxy-2-{[4-(4-isopropoxyphenyl)-1-methyl-1H-indol-3-yl]methylene}-1-benzofuran-3(2H)-one;
ethyl 4-{3-[(Z)-(4,6-dihydroxy-3-oxo-1-benzofuran-2(3H)-ylidene)methyl]-1-methyl-1H-indol-4-yl}benzoate;
N-(4-{3-[(Z)-(4,6-dihydroxy-3-oxo-1-benzofuran-2(3H)-ylidene)methyl]-1-methyl-1H-indol-4-yl}phenyl)acetamide;
(2Z)-2-({4-[4-(dimethylamino)phenyl]-1-methyl-1H-indol-3-yl}methylene)-4,6-dihydroxy-1-benzofuran-3(2H)-one;
(2Z)-4,6-dihydroxy-2-{[1-methyl-4-(6-morpholin-4-ylpyridin-3-yl)-1H-indol-3-yl]methylene}-1-benzofuran-3(2H)-one;
4-{3-[(Z)-(4,6-dihydroxy-3-oxo-1-benzofuran-2(3H)-ylidene)methyl]-1-methyl-1H-indol-4-yl}-N-[3-(dimethylamino)propyl]benzamide;
N-(3-{3-[(Z)-(4,6-dihydroxy-3-oxo-1-benzofuran-2(3H)-ylidene)methyl]-1-methyl-1H-indol-4-yl}phenyl)acetamide;
(2Z)-4,6-dihydroxy-2-({1-methyl-4-[4-(methylamino)phenyl]-1H-indol-3-yl}methylene)-1-benzofuran-3(2H)-one;
(2Z)-4,6-dihydroxy-2-{[4-(3-hydroxyphenyl)-1-methyl-1H-indol-3-yl]methylene}-1-benzofuran-3(2H)-one;
methyl (4-{3-[(Z)-(4,6-dihydroxy-3-oxo-1-benzofuran-2(3H)-ylidene)methyl]-1-methyl-1H-indol-4-yl}phenyl)carbamate;
4-{3-[(Z)-(4,6-dihydroxy-3-oxo-1-benzofuran-2(3H)-ylidene)methyl]-1-methyl-1H-indol-4-yl}-N-methylbenzamide;
1-(4-{3-[(Z)-(4,6-dihydroxy-3-oxo-1-benzofuran-2(3H)-ylidene)methyl]-1-methyl-1H-indol-4-yl}phenyl)-3-methylurea;
3-(4-{3-[(Z)-(4,6-dihydroxy-3-oxo-1-benzofuran-2(3H)-ylidene)methyl]-1-methyl-1H-indol-4-yl}phenyl)-1,1-dimethylurea;
4-{3-[(Z)-(4,6-dihydroxy-3-oxo-1-benzofuran-2(3H)-ylidene)methyl]-1-methyl-1H-indol-4-yl}-N-isopropylbenzamide;
(2Z)-4,6-dihydroxy-2-({1-methyl-4-[4-(pyrrolidin-1-ylcarbonyl)phenyl]-1H-indol-3-yl}methylene)-1-benzofuran-3(2H)-one;
5-{3-[(Z)-(4,6-dihydroxy-3-oxo-1-benzofuran-2(3H)-ylidene)methyl]-1-methyl-1H-indol-4-yl}pyridine-2-carbonitrile;
(2Z)-2-({4-[3-(dimethylamino)phenyl]-1-methyl-1H-indol-3-yl}methylene)-4,6-dihydroxy-1-benzofuran-3(2H)-one;
4-{3-[(Z)-(4,6-dihydroxy-3-oxo-1-benzofuran-2(3H)-ylidene)methyl]-1-methyl-1H-indol-4-yl}-N-(2-furylmethyl)benzamide;
(2Z)-4-hydroxy-2-[(1-methyl-4-phenyl-1H-indol-3-yl)methylene]-1-benzofuran-3(2H)-one;
1-cyclopropyl-3-(4-{3-[(Z)-(4,6-dihydroxy-3-oxo-1-benzofuran-2(3H)-ylidene)methyl]-1-methyl-1H-indol-4-yl}phenyl)urea;
(2Z)-4,6-dihydroxy-2-[(1-methyl-4-{6-[(2-morpholin-4-ylethyl)amino]pyridin-3-yl}-1H-indol-3-yl)methylene]-1-benzofuran-3(2H)-one;
(2Z)-4,6-dihydroxy-2-({1-methyl-4-[1-(2-morpholin-4-ylethyl)-1H-pyrazol-4-yl]-1H-indol-3-yl}methylene)-1-benzofuran-3(2H)-one;
N-(4-{3-[(Z)-(4,6-dihydroxy-3-oxo-1-benzofuran-2(3H)-ylidene)methyl]-1-methyl-1H-indol-4-yl}phenyl)morpholine-4-carboxamide;
methyl [2-({1-[3-(dimethylamino)propyl]-5-methoxy-1H-indol-3-yl}methylene)-3-oxo-2,3-dihydro-1-benzofuran-6-yl]carbamate;
1-[2-({1-[3-(dimethylamino)propyl]-5-methoxy-1H-indol-3-yl}methylene)-3-oxo-2,3-dihydro-1-benzofuran-6-yl]-3-methylurea;
N-[2-({1-[3-(dimethylamino)propyl]-5-methoxy-1H-indol-3-yl}methylene)-3-oxo-2,3-dihydro-1-benzofuran-6-yl]acetamide;
(2Z)-2-[(4-bromo-1-methyl-1H-indol-3-yl)methylene]-4,6-dimethoxy-1-benzofuran-3(2H)-one;
(2Z)-4,6-dimethoxy-2-[(1-methyl-2-phenyl-1H-indol-3-yl)methylene]-1-benzofuran-3(2H)-one;
(2Z)-2-({2-cyclopentyl-5-methoxy-1-[2-(4-methylpiperazin-1-yl)ethyl]-1H-indol-3-yl}methylene)-4,6-dihydroxy-1-benzofuran-3(2H)-one;
(2Z)-2-({2-cyclopentyl-5-methoxy-1-[2-(4-methylpiperazin-1-yl)ethyl]-1H-indol-3-yl}methylene)-6-hydroxy-1-benzofuran-3(2H)-one;
(2Z)-6-hydroxy-2-({5-methoxy-1-[2-(4-methylpiperazin-1-yl)ethyl]-2-(morpholin-4-ylcarbonyl)-1H-indol-3-yl}methylene)-1-benzofuran-3(2H)-one;
(2Z)-6-bromo-2-({1-[3-(dimethylamino)propyl]-5-methoxy-1H-indol-3-yl}methylene)-1-benzofuran-3(2H)-one;
(2Z)-6-hydroxy-2-[1-(5-methoxy-1H-indol-3-yl)ethylidene]-1-benzofuran-3(2H)-one;
tert-butyl [2-({1-[3-(dimethylamino)propyl]-5-methoxy-1H-indol-3-yl}methylene)-3-oxo-2,3-dihydro-1-benzofuran-6-yl]carbamate;
6-amino-2-({1-[3-(dimethylamino)propyl]-5-methoxy-1H-indol-3-yl}methylene)-1-benzofuran-3(2H)-one;
(2Z)-2-{[4-(2-aminophenyl)-1-methyl-1H-indol-3-yl]methylene}-4,6-dihydroxy-1-benzofuran-3(2H)-one;
(2Z)-4,6-dihydroxy-2-{[1-methyl-4-(4-nitrophenyl)-1H-indol-3-yl]methylene}-1-benzofuran-3(2H)-one;
(2Z)-4-hydroxy-6-methoxy-2-[(1-methyl-2-phenyl-1H-indol-3-yl)methylene]-1-benzofuran-3(2H)-one;
N-[2-({1-[3-(dimethylamino)propyl]-5-methoxy-1H-indol-3-yl}methylene)-3-oxo-2,3-dihydro-1-benzofuran-6-yl]methanesulfonamide;
(2Z)-7-bromo-4-methoxy-2-[(1-methyl-4-phenyl-1H-indol-3-yl)methylene]-1-benzofuran-3(2H)-one;
(2Z)-2-({1-[3-(dimethylamino)propyl]-5-methoxy-1H-indol-3-yl}methylene)-6-(hydroxymethyl)-1-benzofuran-3(2H)-one;
4-{3-[(Z)-(4,6-dihydroxy-3-oxo-1-benzofuran-2(3H)-ylidene)methyl]-1-[2-(4-methylpiperazin-1-yl)ethyl]-1H-indol-4-yl}benzamide;
N-{4-[(2Z)-2-({1-[3-(dimethylamino)propyl]-5-methoxy-1H-indol-3-yl}methylene)-3-oxo-2,3-dihydro-1-benzofuran-6-yl]phenyl}acetamide;
(2Z)-6-(2-aminopyrimidin-5-yl)-2-({1-[3-(dimethylamino)propyl]-5-methoxy-1H-indol-3-yl}methylene)-1-benzofuran-3(2H)-one;
(2Z)-7-bromo-4-hydroxy-2-[(1-methyl-4-phenyl-1H-indol-3-yl)methylene]-1-benzofuran-3(2H)-one;
(2Z)-4-bromo-6-hydroxy-2-({5-methoxy-2-methyl-1-[2-(4-methylpiperazin-1-yl)ethyl]-1H-indol-3-yl}methylene)-1-benzofuran-3(2H)-one;
(2Z)-4,6-dihydroxy-2-({5-methoxy-1-[2-(4-methylpiperazin-1-yl)ethyl]-2-(pyrrolidin-1-ylcarbonyl)-1H-indol-3-yl}methylene)-1-benzofuran-3(2H)-one;
(2Z)-6-hydroxy-4-methoxy-2-[(1-methyl-2-phenyl-1H-indol-3-yl)methylene]-1-benzofuran-3(2H)-one;
6-methoxy-2-[(5-methoxy-1H-indol-3-yl)methylene]-1-benzothiophen-3(2H)-one;
2-[(5-methoxy-1H-indol-3-yl)methylene]-6-(methylthio)-1-benzofuran-3(2H)-one;
2-[(5-methoxy-1H-indol-3-yl)methylene]-6-(methylsulfinyl)-1-benzofuran-3(2H)-one;
2-[(5-methoxy-1H-indol-3-yl)methylene]-6-(methylsulfonyl)-1-benzofuran-3(2H)-one;
3-[(Z)-(4,6-dihydroxy-3-oxo-1-benzofuran-2(3H)-ylidene)methyl]-1-methyl-2-phenyl-1H-indole-4-carbonitrile;
(2Z)-2-({4-[4-(dimethylamino)phenyl]-1-[2-(4-methylpiperazin-1-yl)ethyl]-1H-indol-3-yl}methylene)-4,6-dihydroxy-1-benzofuran-3(2H)-one;
(2Z)-4,6-dihydroxy-2-{[1-methyl-4-(4-methylpiperazin-1-yl)-1H-indol-3-yl]methylene}-1-benzofuran-3(2H)-one;
(2Z)-4-hydroxy-2-[(1-methyl-2-phenyl-1H-indol-3-yl)methylene]-1-benzofuran-3(2H)-one;
(2Z)-6-hydroxy-2-[(1-methyl-4-phenyl-1H-indol-3-yl)methylene]-1-benzofuran-3(2H)-one;
2-{2-cyclopropyl-3-[(Z)-(4,6-dihydroxy-3-oxo-1-benzofuran-2(3H)-ylidene)methyl]-5-methoxy-1H-indol-1-yl}-N,N-dimethylacetamide;
(2Z)-2-({2-cyclobutyl-5-methoxy-1-[2-(4-methylpiperazin-1-yl)ethyl]-1H-indol-3-yl}methylene)-4,6-dihydroxy-1-benzofuran-3(2H)-one;
(2Z)-2-({2-cyclohexyl-5-methoxy-1-[2-(4-methylpiperazin-1-yl)ethyl]-1H-indol-3-yl}methylene)-4,6-dihydroxy-1-benzofuran-3(2H)-one;
(2Z)-5-chloro-2-({1-[3-(dimethylamino)propyl]-5-methoxy-2-methyl-1H-indol-3-yl}methylene)-6-hydroxy-1-benzofuran-3(2H)-one;
(2Z)-2-({1-[3-(dimethylamino)propyl]-5-methoxy-2-methyl-1H-indol-3-yl}methylene)-7-fluoro-6-hydroxy-1-benzofuran-3(2H)-one;
(2Z)-2-({1-[3-(dimethylamino)propyl]-5-methoxy-2-methyl-1H-indol-3-yl}methylene)-6-hydroxy-7-methyl-1-benzofuran-3(2H)-one;
(2Z)-7-chloro-6-hydroxy-2-({5-methoxy-2-methyl-1-[2-(4-methylpiperazin-1-yl)ethyl]-1H-indol-3-yl}methylene)-1-benzofuran-3(2H)-one;
(2Z)-6-hydroxy-2-{[5-methoxy-2-methyl-1-(2-pyrrolidin-1-ylethyl)-1H-indol-3-yl]methylene}-1-benzofuran-3(2H)-one;
Z)-6-hydroxy-2-({5-methoxy-2-methyl-1-[2-(2-methylpyrrolidin-1-yl)ethyl]-1H-indol-3-yl}methylene)-1-benzofuran-3(2H)-one;
(2Z)-6-hydroxy-2-{[5-methoxy-2-methyl-1-(2-piperidin-1-ylethyl)-1H-indol-3-yl]methylene}-1-benzofuran-3(2H)-one;
(2Z)-6-hydroxy-2-({5-methoxy-2-methyl-1-[2-(4-methylpiperidin-1-yl)ethyl]-1H-indol-3-yl}methylene)-1-benzofuran-3(2H)-one;
(2Z)-5-chloro-6-hydroxy-2-({5-methoxy-2-methyl-1-[2-(4-methylpiperazin-1-yl)ethyl]-1H-indol-3-yl}methylene)-1-benzofuran-3(2H)-one;
(2Z)-7-fluoro-6-hydroxy-2-({5-methoxy-2-methyl-1-[2-(4-methylpiperazin-1-yl)ethyl]-1H-indol-3-yl}methylene)-1-benzofuran-3(2H)-one;
(2Z)-6-hydroxy-2-({5-methoxy-2-methyl-1-[2-(4-methylpiperazin-1-yl)ethyl]-1H-indol-3-yl}methylene)-5-methyl-1-benzofuran-3(2H)-one;
(2Z)-2-({1-[3-(dimethylamino)propyl]-5-methoxy-2-methyl-1H-indol-3-yl}methylene)-6-hydroxy-5-methyl-1-benzofuran-3(2H)-one;
(2Z)-6-hydroxy-2-({5-methoxy-2-methyl-1-[3-(4-methylpiperidin-1-yl)propyl]-1H-indol-3-yl}methylene)-1-benzofuran-3(2H)-one;
(2Z)-6-hydroxy-2-({5-methoxy-2-methyl-1-[3-(2-methylpyrrolidin-1-yl)propyl]-1H-indol-3-yl}methylene)-1-benzofuran-3(2H)-one;
(2Z)-2-({2-cyclopropyl-1-[3-(dimethylamino)propyl]-5-methoxy-1H-indol-3-yl}methylene)-6-hydroxy-1-benzofuran-3(2H)-one;
(2Z)-4,6-dihydroxy-2-({5-methoxy-1-[2-(4-methylpiperazin-1-yl)ethyl]-2-pyrimidin-5-yl-1H-indol-3-yl}methylene)-1-benzofuran-3(2H)-one;
(2Z)-2-({2-cyclopropyl-5-methoxy-1-[2-(4-methylpiperazin-1-yl)ethyl]-1H-indol-3-yl}methylene)-6-hydroxy-4-methyl-1-benzofuran-3(2H)-one;
(2Z)-2-({1-[3-(dimethylamino)propyl]-5-methoxy-2-methyl-1H-indol-3-yl}methylene)-4-fluoro-6-hydroxy-1-benzofuran-3(2H)-one;
(2Z)-4-chloro-2-({1-[3-(dimethylamino)propyl]-5-methoxy-2-methyl-1H-indol-3-yl}methylene)-6-hydroxy-1-benzofuran-3(2H)-one;
(2Z)-4-chloro-6-hydroxy-2-({5-methoxy-2-methyl-1-[2-(4-methylpiperazin-1-yl)ethyl]-1H-indol-3-yl}methylene)-1-benzofuran-3(2H)-one;
(2Z)-2-({1-[3-(dimethylamino)propyl]-5-methoxy-2-methyl-1H-indol-3-yl}methylene)-6-hydroxy-4-methyl-1-benzofuran-3(2H)-one;
(2Z)-2-({2-(3,5-dimethylisoxazol-4-yl)-5-methoxy-1-[2-(4-methylpiperazin-1-yl)ethyl]-1H-indol-3-yl}methylene)-6-hydroxy-1-benzofuran-3(2H)-one;
Z)-2-{[1-(2-azepan-1-ylethyl)-5-methoxy-2-methyl-1H-indol-3-yl]methylene}-6-hydroxy-1-benzofuran-3(2H)-one;
4-{3-[(4,6-dihydroxy-3-oxo-1-benzofuran-2(3H)-ylidene)methyl]-7-ethyl-5-methoxy-1H-indol-1-yl}butanenitrile;
(2Z)-6-hydroxy-4-methoxy-2-[(5-methoxy-1H-indol-3-yl)methylene]-1-benzofuran-3(2H)-one;
(2Z)-2-({7-ethyl-5-methoxy-1-[2-(4-methylpiperazin-1-yl)ethyl]-1H-indol-3-yl}methylene)-6-hydroxy-1-benzofuran-3(2H)-one;
4-{7-ethyl-3-[(Z)-(6-hydroxy-3-oxo-1-benzofuran-2(3H)-ylidene)methyl]-5-methoxy-1H-indol-1-yl}butanenitrile;
4-{7-ethyl-3-[(Z)-(4-hydroxy-3-oxo-1-benzofuran-2(3H)-ylidene)methyl]-5-methoxy-1H-indol-1-yl}butanenitrile;
(2Z)-2-[(1,4-dimethyl-2-phenyl-1H-indol-3-yl)methylene]-4,6-dihydroxy-1-benzofuran-3(2H)-one;
(2Z)-2-[(1,4-dimethyl-2-pyridin-2-yl-1H-indol-3-yl)methylene]-4,6-dihydroxy-1-benzofuran-3(2H)-one;
(2Z)-4,6-dihydroxy-2-{[1-(2-hydroxyethyl)-4-phenyl-1H-indol-3-yl]methylene}-1-benzofuran-3(2H)-one;
(2Z)-6-hydroxy-2-[(5-methoxy-7-pyridin-4-yl-1H-indol-3-yl)methylene]-1-benzofuran-3(2H)-one;
(2Z)-6-hydroxy-2-[(5-methoxy-7-pyridin-3-yl-1H-indol-3-yl)methylene]-1-benzofuran-3(2H)-one;
(2Z)-2-[(5-bromo-1H-indol-3-yl)methylene]-7-methoxy-1-benzofuran-3(2H)-one;
7-hydroxy-2-[(5-methoxy-1H-indol-3-yl)methylene]-1-benzofuran-3(2H)-one;
7-methoxy-2-[(5-methoxy-1H-indol-3-yl)methylene]-1-benzofuran-3(2H)-one;
1-{(2Z)-2-[(5-methoxy-1H-indol-3-yl)methylene]-3-oxo-2,3-dihydro-1-benzofuran-5-yl}-3-pyridin-3-ylurea;
1-{(2Z)-2-[(5-methoxy-1H-indol-3-yl)methylene]-3-oxo-2,3-dihydro-1-benzofuran-5-yl}-3-phenylurea;
1-isopropyl-3-{(2Z)-2-[(5-methoxy-1H-indol-3-yl)methylene]-3-oxo-2,3-dihydro-1-benzofuran-5-yl}urea;
1-butyl-3-{(2Z)-2-[(5-methoxy-1H-indol-3-yl)methylene]-3-oxo-2,3-dihydro-1-benzofuran-5-yl}urea;
1-cyclohexyl-3-{(2Z)-2-[(5-methoxy-1H-indol-3-yl)methylene]-3-oxo-2,3-dihydro-1-benzofuran-5-yl}urea;
1-ethyl-3-{(2Z)-2-[(5-methoxy-1H-indol-3-yl)methylene]-3-oxo-2,3-dihydro-1-benzofuran-5-yl}urea;
methyl ({(2Z)-2-[(5-methoxy-1H-indol-3-yl)methylene]-3-oxo-2,3-dihydro-1-benzofuran-5-yl}carbamoyl)carbamate;
1-{(2Z)-2-[(5-methoxy-1H-indol-3-yl)methylene]-3-oxo-2,3-dihydro-1-benzofuran-5-yl}-3-(4-methoxyphenyl)urea;
1-[4-(dimethylamino)phenyl]-3-{(2Z)-2-[(5-methoxy-1H-indol-3-yl)methylene]-3-oxo-2,3-dihydro-1-benzofuran-5-yl}urea;
4-{3-[(Z)-(4,6-dihydroxy-3-oxo-1-benzofuran-2(3H)-ylidene)methyl]-2-ethyl-5-methoxy-1H-indol-1-yl}butanenitrile;
2-[(5-methoxy-1H-indol-3-yl)methylene]-3-oxo-2,3-dihydro-1-benzofuran-6-yl trifluoromethanesulfonate;
2-[(5-methoxy-1H-indol-3-yl)methylene]-3-oxo-2,3-dihydro-1-benzofuran-6-carboxamide;
(2Z)-4,6-dihydroxy-2-[(1-methyl-4-morpholin-4-yl-1H-indol-3-yl)methylene]-1-benzofuran-3(2H)-one;
(2Z)-4,6-dihydroxy-2-[(4-methoxy-1-methyl-2-phenyl-1H-indol-3-yl)methylene]-1-benzofuran-3(2H)-one;
(2Z)-6-hydroxy-2-[(5-methoxy-7-pyrimidin-5-yl-1H-indol-3-yl)methylene]-1-benzofuran-3(2H)-one;
(2Z)-4,6-dihydroxy-2-[(1-methyl-4-nitro-2-phenyl-1H-indol-3-yl)methylene]-1-benzofuran-3(2H)-one;
4-{3-[(Z)-(6-hydroxy-3-oxo-1-benzofuran-2(3H)-ylidene)methyl]-5-methoxy-7-pyridin-4-yl-1H-indol-1-yl}butanenitrile;
4-{3-[(Z)-(6-hydroxy-3-oxo-1-benzofuran-2(3H)-ylidene)methyl]-5-methoxy-7-pyrimidin-5-yl-1H-indol-1-yl}butanenitrile;
4-{3-[(Z)-(6-hydroxy-3-oxo-1-benzofuran-2(3H)-ylidene)methyl]-5-methoxy-7-pyridin-3-yl-1H-indol-1-yl}butanenitrile;
4-{3-[(Z)-(4,6-dihydroxy-3-oxo-1-benzofuran-2(3H)-ylidene)methyl]-5-methoxy-7-pyrimidin-5-yl-1H-indol-1-yl}butanenitrile;
(2Z)-2-[(5-methoxy-1H-indol-3-yl)methylene]-3-oxo-2,3-dihydro-1-benzofuran-6-carbonitrile;
(2Z)-2-[(5-methoxy-1H-indol-3-yl)methylene]-6-(2H-tetrazol-5-yl)-1-benzofuran-3(2H)-one;
4-{3-[(Z)-(4,6-dihydroxy-3-oxo-1-benzofuran-2(3H)-ylidene)methyl]-5-methoxy-1Hindol-1-yl}butanenitrile;
4-{3-[(Z)-(6-hydroxy-3-oxo-1-benzofuran-2(3H)-ylidene)methyl]-5-methoxy-1Hindol-1-yl}butanenitrile;
(2Z)-2-({7-ethyl-5-methoxy-1-[2-(4-methylpiperazin-1-yl)ethyl]-1H-indol-3-yl}methylene)-4,6-dihydroxy-1-benzofuran-3(2H)-one;
4-{3-[(Z)-(5-hydroxy-3-oxo-1-benzofuran-2(3H)-ylidene)methyl]-5-methoxy-1Hindol-1-yl}butanenitrile;
(2Z)-2-({1-[3-(dimethylamino)propyl]-5-methoxy-1H-indol-3-yl}methylene)-5-hydroxy-1-benzofuran-3(2H)-one;
(2Z)-5-bromo-2-({1-[3-(dimethylamino)propyl]-5-methoxy-1H-indol-3-yl}methylene)-1-benzofuran-3(2H)-one;
2Z)-5-hydroxy-2-({5-methoxy-2-methyl-1-[2-(4-methylpiperazin-1-yl)ethyl]-1H-indol-3-yl}methylene)-1-benzofuran-3(2H)-one;
1-[(2Z)-2-({1-[3-(dimethylamino)propyl]-5-methoxy-1H-indol-3-yl}methylene)-3-oxo-2,3-dihydro-1-benzofuran-5-yl]-3-methylurea;
(2Z)-5-bromo-2-[(5-methoxy-1H-indol-3-yl)methylene]-1-benzofuran-3(2H)-one;
(2Z)-2-[(5-methoxy-1H-indol-3-yl)methylene]-3-oxo-2,3-dihydro-1-benzofuran-5-carbonitrile;
(2Z)-2-[(5-methoxy-1H-indol-3-yl)methylene]-5-(1H-tetrazol-5-yl)-1-benzofuran-3(2H)-one;
N-[(2Z)-2-({1-[3-(dimethylamino)propyl]-5-methoxy-1H-indol-3-yl}methylene)-3-oxo-2,3-dihydro-1-benzofuran-5-yl]acetamide;
(2Z)-5-bromo-2-[(1-methyl-4-phenyl-1H-indol-3-yl)methylene]-1-benzofuran-3(2H)-one;
1-methyl-3-{(2Z)-2-[(1-methyl-4-phenyl-1H-indol-3-yl)methylene]-3-oxo-2,3-dihydro-1-benzofuran-5-yl}urea;
(2Z)-5,6-dihydroxy-2-[(5-methoxy-1H-indol-3-yl)methylene]-1-benzofuran-3(2H)-one;
(2Z)-5-hydroxy-2-[(1-methyl-4-phenyl-1H-indol-3-yl)methylene]-1-benzofuran-3(2H)-one;
(2Z)-4,6-dihydroxy-2-[(1-methyl-4-phenyl-1H-indol-3-yl)methylene]-1-benzofuran-3(2H)-one;
tert-butyl {(2Z)-2-[(1-methyl-4-phenyl-1H-indol-3-yl)methylene]-3-oxo-2,3-dihydro-1-benzofuran-5-yl}carbamate;
1-[(2Z)-2-({2-cyclohexyl-5-methoxy-1-[2-(4-methylpiperazin-1-yl)ethyl]-1H-indol-3-yl}methylene)-3-oxo-2,3-dihydro-1-benzofuran-5-yl]-3-methylurea;
(2Z)-5-amino-2-[(1-methyl-4-phenyl-1H-indol-3-yl)methylene]-1-benzofuran-3(2H)-one;
1-{(2Z)-2-[(1-methyl-4-phenyl-1H-indol-3-yl)methylene]-3-oxo-2,3-dihydro-1-benzofuran-5-yl}-3-pyridin-3-ylurea;
1-{(2Z)-2-[(5-methoxy-1H-indol-3-yl)methylene]-3-oxo-2,3-dihydro-1-benzofuran-5-yl}-3-methylurea;
1-[(2Z)-2-({1-[3-(dimethylamino)propyl]-5-methoxy-1H-indol-3-yl}methylene)-3-oxo-2,3-dihydro-1-benzofuran-5-yl]urea;
methyl [(2Z)-2-({1-[3-(dimethylamino)propyl]-5-methoxy-1H-indol-3-yl}methylene)-3-oxo-2,3-dihydro-1-benzofuran-5-yl]carbamate;
(2Z)-2-({1-[3-(dimethylamino)propyl]-5-methoxy-1H-indol-3-yl}methylene)-Nmethyl-3-oxo-2,3-dihydro-1-benzofuran-5-carboxamide;
1-[(2Z)-2-({1-[3-(dimethylamino)propyl]-5-methoxy-1H-indol-3-yl}methylene)-3-oxo-2,3-dihydro-1-benzofuran-5-yl]-3-(4-methoxyphenyl)urea;
(2Z)-5-(hydroxymethyl)-2-({5-methoxy-1-[2-(4-methylpiperazin-1-yl)ethyl]-1Hindol-3-yl}methylene)-1-benzofuran-3(2H)-one;
1-ethyl-3-{(2Z)-2-[(1-methyl-4-phenyl-1H-indol-3-yl)methylidene]-3-oxo-2,3-dihydro-1-benzofuran-5-yl}urea;
(2E)-4,6-dihydroxy-2-[(1-methyl-4-phenyl-1H-indol-3-yl)methylene]-1-benzofuran-3(2H)-one-(2Z)-4,6-dihydroxy-2-[(1-methyl-4-phenyl-1H-indol-3-yl)methylene]-1-benzofuran-3(2H)-one (1:1);
(2Z)-2-({5-methoxy-1-[2-(4-methylpiperazin-1-yl)ethyl]-1H-indol-3-yl}methylene)-3-oxo-2,3-dihydro-1-benzofuran-5-yl methylcarbamate;
1-[(2Z)-2-({1-[3-(dimethylamino)propyl]-5-methoxy-1H-indol-3-yl}methylene)-3-oxo-2,3-dihydro-1-benzofuran-5-yl]-3-ethylurea;
1-[(2Z)-2-({5-methoxy-1-[2-(4-methylpiperazin-1-yl)ethyl]-1H-indol-3-yl}methylene)-3-oxo-2,3-dihydro-1-benzofuran-5-yl]-3-prop-2-yn-1-ylurea;
1-(2-aminoethyl)-3-[(2Z)-2-({5-methoxy-1-[2-(4-methylpiperazin-1-yl)ethyl]-1Hindol-3-yl}methylene)-3-oxo-2,3-dihydro-1-benzofuran-5-yl]urea;
1-allyl-3-[(2Z)-2-({5-methoxy-1-[2-(4-methylpiperazin-1-yl)ethyl]-1H-indol-3-yl}methylene)-3-oxo-2,3-dihydro-1-benzofuran-5-yl]urea;
1-[(2Z)-2-({5-methoxy-1-[2-(4-methylpiperazin-1-yl)ethyl]-1H-indol-3-yl}methylene)-3-oxo-2,3-dihydro-1-benzofuran-5-yl]urea;
1-azetidin-3-yl-3-[(2Z)-2-({5-methoxy-1-[2-(4-methylpiperazin-1-yl)ethyl]-1Hindol-3-yl}methylene)-3-oxo-2,3-dihydro-1-benzofuran-5-yl]urea;
1-[(2Z)-2-({5-methoxy-1-[2-(4-methylpiperazin-1-yl)ethyl]-1H-indol-3-yl}methylene)-3-oxo-2,3-dihydro-1-benzofuran-5-yl]-3-methylurea;
1-[(2Z)-2-({1-[3-(dimethylamino)propyl]-5-methoxy-1H-indol-3-yl}methylene)-3-oxo-2,3-dihydro-1-benzofuran-5-yl]-3-(2-hydroxyethyl)urea;
1-[(2Z)-2-({5-methoxy-2-methyl-1-[2-(4-methylpiperazin-1-yl)ethyl]-1H-indol-3-yl}methylene)-3-oxo-2,3-dihydro-1-benzofuran-5-yl]-3-methylurea;
(2E)-4,6-dihydroxy-2-[(1-methyl-4-phenyl-1H-indol-3-yl)methylene]-1-benzofuran-3(2H)-one;
(2Z)-2-({5-methoxy-2-methyl-1-[2-(4-methylpiperazin-1-yl)ethyl]-1H-indol-3-yl}methylene)-3-oxo-2,3-dihydro-1-benzofuran-6-yl methyl(phenyl)carbamate;
1-[(2Z)-2-({2-cyclopropyl-5-methoxy-1-[2-(4-methylpiperazin-1-yl)ethyl]-1Hindol-3-yl}methylene)-3-oxo-2,3-dihydro-1-benzofuran-5-yl]-3-methylurea;
1-cyclopropyl-3-[(2Z)-2-({1-[3-(dimethylamino)propyl]-5-methoxy-1H-indol-3-yl}methylene)-3-oxo-2,3-dihydro-1-benzofuran-5-yl]urea;
1-[(2Z)-2-({2-cyclopentyl-5-methoxy-1-[2-(4-methylpiperazin-1-yl)ethyl]-1Hindol-3-yl}methylene)-3-oxo-2,3-dihydro-1-benzofuran-5-yl]-3-methylurea;
1-[(2Z)-2-({2-cyclobutyl-5-methoxy-1-[2-(4-methylpiperazin-1-yl)ethyl]-1H-indol-3-yl}methylene)-3-oxo-2,3-dihydro-1-benzofuran-5-yl]-3-methylurea;
1-[(2Z)-2-({5-methoxy-1-[2-(4-methylpiperazin-1-yl)ethyl]-1H-indol-3-yl}methylene)-3-oxo-2,3-dihydro-1-benzofuran-5-yl]-3-[2-(methylamino)ethyl]urea;
1-[(2Z)-2-({5-methoxy-1-[2-(4-methylpiperazin-1-yl)ethyl]-1H-indol-3-yl}methylene)-3-oxo-2,3-dihydro-1-benzofuran-5-yl]-3-[3-(methylamino)propyl]urea;
1-(4-aminobutyl)-3-[(2Z)-2-({5-methoxy-1-[2-(4-methylpiperazin-1-yl)ethyl]-1Hindol-3-yl}methylene)-3-oxo-2,3-dihydro-1-benzofuran-5-yl]urea;
1-(3-aminopropyl)-3-[(2Z)-2-({5-methoxy-1-[2-(4-methylpiperazin-1-yl)ethyl]-1Hindol-3-yl}methylene)-3-oxo-2,3-dihydro-1-benzofuran-5-yl]urea;
1-[(2Z)-2-({5-methoxy-7-[(1E)-3-morpholin-4-ylprop-1-en-1-yl]-1H-indol-3-yl}methylene)-3-oxo-2,3-dihydro-1-benzofuran-5-yl]-3-methylurea;
1-[(2Z)-2-({1-[3-(dimethylamino)propyl]-5-methoxy-1H-indol-3-yl}methylene)-3-oxo-2,3-dihydro-1-benzofuran-5-yl]-3-(3-hydroxypropyl)urea;
1-[3-(dimethylamino)propyl]-3-[(2Z)-2-({5-methoxy-1-[2-(4-methylpiperazin-1-yl)ethyl]-1H-indol-3-yl}methylene)-3-oxo-2,3-dihydro-1-benzofuran-5-yl]urea;
1-[(2Z)-2-{[5-methoxy-7-(morpholin-4-ylmethyl)-1H-indol-3-yl]methylene}-3-oxo-2,3-dihydro-1-benzofuran-5-yl]-3-methylurea;
1-[(2Z)-2-({5-methoxy-7-[(4-methylpiperazin-1-yl)methyl]-1H-indol-3-yl}methylene)-3-oxo-2,3-dihydro-1-benzofuran-5-yl]-3-methylurea;
1-[(2Z)-2-({5-methoxy-1-methyl-7-[3-(4-methylpiperazin-1-yl)propyl]-1H-indol-3-yl}methylene)-3-oxo-2,3-dihydro-1-benzofuran-5-yl]-3-methylurea;
1-[(2Z)-2-({7-[(1E)-3-(dimethylamino)prop-1-en-1-yl]-5-methoxy-1H-indol-3-yl}methylene)-3-oxo-2,3-dihydro-1-benzofuran-5-yl]-3-methylurea;
1-[2-(dimethylamino)ethyl]-3-[(2Z)-2-({5-methoxy-1-[2-(4-methylpiperazin-1-yl)ethyl]-1H-indol-3-yl}methylene)-3-oxo-2,3-dihydro-1-benzofuran-5-yl]urea;
1-[(2Z)-2-({5-methoxy-1-[2-(4-methylpiperazin-1-yl)ethyl]-2-phenyl-1H-indol-3-yl}methylene)-3-oxo-2,3-dihydro-1-benzofuran-5-yl]-3-methylurea;
1-(2-aminoethyl)-3-[(2Z)-2-({5-methoxy-1-[2-(4-methylpiperazin-1-yl)ethyl]-2-phenyl-1H-indol-3-yl}methylene)-3-oxo-2,3-dihydro-1-benzofuran-5-yl]urea;
1-(2-aminoethyl)-3-[(2Z)-2-({2-cyclopentyl-5-methoxy-1-[2-(4-methylpiperazin-1-yl)ethyl]-1H-indol-3-yl}methylene)-3-oxo-2,3-dihydro-1-benzofuran-5-yl]urea;
1-[(2Z)-2-{[5-methoxy-1-(3-piperidin-1-ylpropyl)-1H-indol-3-yl]methylene}-3-oxo-2,3-dihydro-1-benzofuran-5-yl]-3-methylurea;
1-[(2Z)-2-{[1-(3-cyanopropyl)-5-methoxy-1H-indol-3-yl]methylene}-3-oxo-2,3-dihydro-1-benzofuran-5-yl]-3-methylurea;
1-[(2Z)-2-{[5-methoxy-1-(3-morpholin-4-ylpropyl)-1H-indol-3-yl]methylene}-3-oxo-2,3-dihydro-1-benzofuran-5-yl]-3-methylurea;
1-[(2Z)-2-{[2-(3,5-dimethylisoxazol-4-yl)-5-methoxy-1H-indol-3-yl]methylene}-3-oxo-2,3-dihydro-1-benzofuran-5-yl]-3-methylurea;
1-[(2Z)-2-({5-methoxy-7-[3-(4-methylpiperazin-1-yl)propyl]-1H-indol-3-yl}methylene)-3-oxo-2,3-dihydro-1-benzofuran-5-yl]-3-methylurea;
1-[(2Z)-2-({2-cyclohexyl-5-methoxy-1-[2-(4-methylpiperazin-1-yl)ethyl]-1H-indol-3-yl}methylene)-3-oxo-2,3-dihydro-1-benzofuran-5-yl]-3-[2-(methylamino)ethyl]urea;
1-[(2Z)-2-{[5-methoxy-2-methyl-1-(3-morpholin-4-ylpropyl)-1H-indol-3-yl]methylene}-3-oxo-2,3-dihydro-1-benzofuran-5-yl]-3-methylurea;
1-[(2Z)-2-({5-methoxy-2-methyl-1-[3-(4-methylpiperazin-1-yl)propyl]-1H-indol-3-yl}methylene)-3-oxo-2,3-dihydro-1-benzofuran-5-yl]-3-methylurea;
1-[(2Z)-2-({1-[3-(4-hydroxypiperidin-1-yl)propyl]-5-methoxy-2-methyl-1H-indol-3-yl}methylene)-3-oxo-2,3-dihydro-1-benzofuran-5-yl]-3-methylurea;
1-[(2Z)-2-{[5-methoxy-2-methyl-1-(3-piperidin-1-ylpropyl)-1H-indol-3-yl]methylene}-3-oxo-2,3-dihydro-1-benzofuran-5-yl]-3-methylurea;
1-[(2Z)-2-({1-[3-(3-hydroxypyrrolidin-1-yl)propyl]-5-methoxy-2-methyl-1H-indol-3-yl}methylene)-3-oxo-2,3-dihydro-1-benzofuran-5-yl]-3-methylurea;
1-[(2Z)-2-({5-methoxy-1-[2-(4-methylpiperazin-1-yl)ethyl]-2-pyridin-4-yl-1Hindol-3-yl}methylene)-3-oxo-2,3-dihydro-1-benzofuran-5-yl]-3-methylurea;
1-[(2Z)-2-{[2-(3-isopropyl-1,2,4-oxadiazol-5-yl)-5-methoxy-1H-indol-3-yl]methylene}-3-oxo-2,3-dihydro-1-benzofuran-5-yl]-3-methylurea;
1-[(2Z)-2-{[2-(3-cyclopropyl-1,2,4-oxadiazol-5-yl)-5-methoxy-1H-indol-3-yl]methylene}-3-oxo-2,3-dihydro-1-benzofuran-5-yl]-3-methylurea;
1-[(2Z)-2-{[5-methoxy-2-(3-propyl-1,2,4-oxadiazol-5-yl)-1H-indol-3-yl]methylene}-3-oxo-2,3-dihydro-1-benzofuran-5-yl]-3-methylurea;
1-[(2Z)-2-({5-methoxy-7-[(1E)-3-(4-methylpiperazin-1-yl)prop-1-en-1-yl]-1Hindol-3-yl}methylene)-3-oxo-2,3-dihydro-1-benzofuran-5-yl]-3-methylurea;
1-{(2Z)-2-[(7-cyano-5-methoxy-1H-indol-3-yl)methylene]-3-oxo-2,3-dihydro-1-benzofuran-5-yl}-3-methylurea;
1-[(2Z)-2-({5-methoxy-1-[2-(4-methylpiperazin-1-yl)ethyl]-2-pyridin-3-yl-1Hindol-3-yl}methylene)-3-oxo-2,3-dihydro-1-benzofuran-5-yl]-3-methylurea;
1-[(2Z)-2-{[5-methoxy-2-methyl-1-(3-pyrrolidin-1-ylpropyl)-1H-indol-3-yl]methylidene}-3-oxo-2,3-dihydro-1-benzofuran-5-yl]-3-methylurea;
1-[(2Z)-2-({1-[3-(1H-imidazol-1-yl)propyl]-5-methoxy-2-methyl-1H-indol-3-yl}methylidene)-3-oxo-2,3-dihydro-1-benzofuran-5-yl]-3-methylurea;
1-{(2Z)-2-[(1-{3-[4-(2-hydroxyethyl)piperazin-1-yl]propyl}-5-methoxy-2-methyl-1H-indol-3-yl)methylidene]-3-oxo-2,3-dihydro-1-benzofuran-5-yl}-3-methylurea;
5-methoxy-N,N-dimethyl-3-[(Z)-{5-[(methylcarbamoyl)amino]-3-oxo-1-benzofuran-2(3H)-ylidene}methyl]-1H-indole-2-carboxamide;
1-[(2Z)-2-({5-methoxy-2-[(4-methylpiperazin-1-yl)carbonyl]-1H-indol-3-yl}methylidene)-3-oxo-2,3-dihydro-1-benzofuran-5-yl]-3-methylurea;
N-[2-(dimethylamino)ethyl]-5-methoxy-N-methyl-3-[(Z)-{5-[(methylcarbamoyl)amino]-3-oxo-1-benzofuran-2(3H)-ylidene}methyl]-1H-indole-2-carboxamide;
5-methoxy-N-methyl-3-[(Z)-{5-[(methylcarbamoyl)amino]-3-oxo-1-benzofuran-2(3H)-ylidene}methyl]-1H-indole-2-carboxamide;
1-{(2Z)-2-[(2-cyano-5-methoxy-1H-indol-3-yl)methylidene]-3-oxo-2,3-dihydro-1-benzofuran-5-yl}-3-methylurea;
N-[2-(dimethylamino)ethyl]-5-methoxy-3-[(Z)-{5-[(methylcarbamoyl)amino]-3-oxo-1-benzofuran-2(3H)-ylidene}methyl]-1H-indole-2-carboxamide;
1-[(2Z)-2-{[5-methoxy-2-(1,2,3,6-tetrahydropyridin-4-yl)-1H-indol-3-yl]methylidene}-3-oxo-2,3-dihydro-1-benzofuran-5-yl]-3-methylurea;
1-[(2Z)-2-({5-methoxy-1-[2-(4-methylpiperazin-1-yl)ethyl]-2-(1,2,3,6-tetrahydropyridin-4-yl)-1H-indol-3-yl}methylidene)-3-oxo-2,3-dihydro-1-benzofuran-5-yl]-3-methylurea;
1-[(2Z)-2-{[5-methoxy-2-methyl-1-(2-morpholin-4-ylethyl)-1H-indol-3-yl]methylidene}-3-oxo-2,3-dihydro-1-benzofuran-5-yl]-3-methylurea;
1-[(2Z)-2-{[5-methoxy-2-methyl-1-(2-piperidin-1-ylethyl)-1H-indol-3-yl]methylidene}-3-oxo-2,3-dihydro-1-benzofuran-5-yl]-3-methylurea;
1-[(2Z)-2-({1-[2-(dimethylamino)ethyl]-5-methoxy-2-methyl-1H-indol-3-yl}methylidene)-3-oxo-2,3-dihydro-1-benzofuran-5-yl]-3-methylurea;
1-[(2Z)-2-{[5-methoxy-2-methyl-1-(2-pyrrolidin-1-ylethyl)-1H-indol-3-yl]methylidene}-3-oxo-2,3-dihydro-1-benzofuran-5-yl]-3-methylurea;
1-[(2Z)-2-({2-[(dimethylamino)methyl]-5-methoxy-1H-indol-3-yl}methylidene)-3-oxo-2,3-dihydro-1-benzofuran-5-yl]-3-methylurea;
1-[(2Z)-2-{[2-([2-(dimethylamino)ethyl](methyl)aminomethyl)-5-methoxy-1Hindol-3-yl]methylidene}-3-oxo-2,3-dihydro-1-benzofuran-5-yl]-3-methylurea;
1-[(2Z)-2-({5-methoxy-2-[3-(1-methylethyl)-1,2,4-oxadiazol-5-yl]-1-[2-(4-methylpiperazin-1-yl)ethyl]-1H-indol-3-yl}methylidene)-3-oxo-2,3-dihydro-1-benzofuran-5-yl]-3-methylurea;
1-[(2Z)-2-({5-methoxy-2-[(4-methylpiperazin-1-yl)methyl]-1H-indol-3-yl}methylidene)-3-oxo-2,3-dihydro-1-benzofuran-5-yl]-3-methylurea;
1-[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]-3-methylurea;
1-[(2Z)-2-({5-methoxy-2-methyl-1-[2-(4-methylpiperazin-1-yl)ethyl]-1H-indol-3-yl}methylidene)-3-oxo-2,3-dihydro-1-benzofuran-5-yl]-3-pyridin-3-ylurea;
1-[(2Z)-2-({5-methoxy-2-methyl-1-[2-(4-methylpiperazin-1-yl)ethyl]-1H-indol-3-yl}methylene)-3-oxo-2,3-dihydro-1-benzofuran-5-yl]-3-{4-[(4-methylpiperazin-1-yl)carbonyl]phenyl}urea;
1-[(2Z)-2-{[1-(2-hydroxyethyl)-5-methoxy-2-methyl-1H-indol-3-yl]methylene}-3-oxo-2,3-dihydro-1-benzofuran-5-yl]-3-{4-[(4-methylpiperazin-1-yl)carbonyl]phenyl}urea;
1-{(2Z)-2-[(1-ethyl-5-methoxy-2-methyl-1H-indol-3-yl)methylene]-3-oxo-2,3-dihydro-1-benzofuran-5-yl}-3-{4-[(4-methylpiperazin-1-yl)carbonyl]phenyl}urea;
1-[(2Z)-2-({5-methoxy-2-[3-(1-methylethyl)-1,2,4-oxadiazol-5-yl]-1-[2-(4-methylpiperazin-1-yl)ethyl]-1H-indol-3-yl}methylidene)-3-oxo-2,3-dihydro-1-benzofuran-5-yl]-3-methylurea;
N-(2-hydroxy-1,1-dimethylethyl)-5-methoxy-3-[(Z)-{5-[(methylcarbamoyl)amino]-3-oxo-1-benzofuran-2(3H)-ylidene}methyl]-1H-indole-2-carboxamide;
1-[(2Z)-2-({2-(3,5-dimethylisoxazol-4-yl)-5-methoxy-1-[2-(4-methylpiperazin-1-yl)ethyl]-1H-indol-3-yl}methylidene)-3-oxo-2,3-dihydro-1-benzofuran-5-yl]-3-methylurea;
1-{(2Z)-2-[(2-{4-[(dimethylamino)methyl]phenyl}-5-methoxy-1H-indol-3-yl)methylidene]-3-oxo-2,3-dihydro-1-benzofuran-5-yl}-3-methylurea;
1-[(2Z)-2-{[2-(3,5-dimethyl-1H-pyrazol-4-yl)-5-methoxy-1H-indol-3-yl]methylidene}-3-oxo-2,3-dihydro-1-benzofuran-5-yl]-3-methylurea;
1-[(2Z)-2-({2-(3-cyclopropyl-1,2,4-oxadiazol-5-yl)-5-methoxy-1-[2-(4-methylpiperazin-1-yl)ethyl]-1H-indol-3-yl}methylidene)-3-oxo-2,3-dihydro-1-benzofuran-5-yl]-3-methylurea;
1-[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]-3-methylurea;
1-[(2Z)-2-({2-cyano-5-methoxy-1-[2-(4-methylpiperazin-1-yl)ethyl]-1H-indol-3-yl}methylidene)-3-oxo-2,3-dihydro-1-benzofuran-5-yl]-3-methylurea;
1-{(2Z)-2-[(2-{3-[(dimethylamino)methyl]phenyl}-5-methoxy-1H-indol-3-yl)methylidene]-3-oxo-2,3-dihydro-1-benzofuran-5-yl}-3-methylurea;
1-{(2Z)-2-[(2-{4-[(dimethylamino)methyl]phenyl}-5-methoxy-1-[2-(4-methylpiperazin-1-yl)ethyl]-1H-indol-3-yl)methylidene]-3-oxo-2,3-dihydro-1-benzofuran-5-yl}-3-methylurea;
1-{(2Z)-2-[(2-tert-butyl-5-methoxy-1H-indol-3-yl)methylidene]-3-oxo-2,3-dihydro-1-benzofuran-5-yl}-3-methylurea;
1-[(2Z)-2-({5-methoxy-2-methyl-1-[2-(4-methylpiperazin-1-yl)ethyl]-1H-indol-3-yl}methylidene)-3-oxo-2,3-dihydro-1-benzofuran-5-yl]-3-pyridin-3-ylurea;
1-[4-(dimethylamino)phenyl]-3-[(2Z)-2-({5-methoxy-2-methyl-1-[2-(4-methylpiperazin-1-yl)ethyl]-1H-indol-3-yl}methylidene)-3-oxo-2,3-dihydro-1-benzofuran-5-yl]urea;
1-[(2Z)-2-{[1-(3-cyanopropyl)-5-methoxy-2-(1,3,5-trimethyl-1H-pyrazol-4-yl)-1H-indol-3-yl]methylene}-3-oxo-2,3-dihydro-1-benzofuran-5-yl]-3-methylurea;
1-[(2Z)-2-{[5-methoxy-2-(1,3,5-trimethyl-1H-pyrazol-4-yl)-1H-indol-3-yl]methylene}-3-oxo-2,3-dihydro-1-benzofuran-5-yl]-3-pyridin-3-ylurea;
1-[(2Z)-2-({5-methoxy-2-(4-methylpiperazin-1-yl)-1-[2-(4-methylpiperazin-1-yl)ethyl]-1H-indol-3-yl}methylene)-3-oxo-2,3-dihydro-1-benzofuran-5-yl]-3-methylurea;
1-[(2Z)-2-{[2-(2,6-dimethoxyphenyl)-5-methoxy-1H-indol-3-yl]methylene}-3-oxo-2,3-dihydro-1-benzofuran-5-yl]-3-methylurea;
1-[(2Z)-2-({2-cyclopentyl-5-methoxy-1-[2-(4-methylpiperazin-1-yl)ethyl]-1H-indol-3-yl}methylene)-3-oxo-2,3-dihydro-1-benzofuran-5-yl]-3-pyridin-3-ylurea;
N-[2-(dimethylamino)ethyl]-4-[({(2Z)-2-[(1-ethyl-5-methoxy-2-methyl-1H-indol-3-yl)methylene]-3-oxo-2,3-dihydro-1-benzofuran-5-yl}carbamoyl)amino]-N-methylbenzamide;
1-[(2Z)-2-({5-methoxy-2-methyl-1-[2-(4-methylpiperazin-1-yl)ethyl]-1H-indol-3-yl}methylene)-6-methyl-3-oxo-2,3-dihydro-1-benzofuran-5-yl]-3-methylurea;
1-{(2Z)-2-[(2-cyclohexyl-5-methoxy-1H-indol-3-yl)methylene]-3-oxo-2,3-dihydro-1-benzofuran-5-yl}-3-methylurea;
1-{(2Z)-2-[(1-ethyl-5-methoxy-1H-indol-3-yl)methylene]-3-oxo-2,3-dihydro-1-benzofuran-5-yl}-3-{4-[(4-methylpiperazin-1-yl)carbonyl]phenyl}urea;
1-{(2Z)-2-[(5-methoxy-1H-indol-3-yl)methylene]-3-oxo-2,3-dihydro-1-benzofuran-5-yl}-3-{4-[(4-methylpiperazin-1-yl)carbonyl]phenyl}urea;
1-{(2Z)-2-[(2-cyclohexyl-5-methoxy-1H-indol-3-yl)methylene]-3-oxo-2,3-dihydro-1-benzofuran-5-yl}-3-{4-[(4-methylpiperazin-1-yl)carbonyl]phenyl}urea;
1-{(2Z)-2-[(2-cyclohexyl-1-ethyl-5-methoxy-1H-indol-3-yl)methylene]-3-oxo-2,3-dihydro-1-benzofuran-5-yl}-3-{4-[(4-methylpiperazin-1-yl)carbonyl]phenyl}urea;
1-[(2Z)-2-({5-methoxy-1-[2-(4-methylpiperazin-1-yl)ethyl]-2-(1,3,5-trimethyl-1H-pyrazol-4-yl)-1H-indol-3-yl}methylene)-3-oxo-2,3-dihydro-1-benzofuran-5-yl]-3-methylurea;
1-[(2Z)-2-({5-methoxy-2-methyl-1-[2-(4-methylpiperazin-1-yl)ethyl]-1H-indol-3-yl}methylene)-3-oxo-2,3-dihydro-1-benzofuran-5-yl]-3-methylthiourea;
1-[(2Z)-2-{[1-{2-[(2-hydroxyethyl)amino]ethyl}-5-methoxy-2-(1,3,5-trimethyl-1H-pyrazol-4-yl)-1H-indol-3-yl]methylene}-3-oxo-2,3-dihydro-1-benzofuran-5-yl]-3-methylurea;
N-[2-(dimethylamino)ethyl]-4-({[(2Z)-2-({5-methoxy-2-methyl-1-[2-(4-methylpiperazin-1-yl)ethyl]-1H-indol-3-yl}methylene)-3-oxo-2,3-dihydro-1-benzofuran-5-yl]carbamoyl}amino)-N-methylbenzamide;
1-(4-{[4-(dimethylamino)piperidin-1-yl]carbonyl}phenyl)-3-{(2Z)-2-[(1-ethyl-5-methoxy-2-methyl-1H-indol-3-yl)methylene]-3-oxo-2,3-dihydro-1-benzofuran-5-yl}urea;
1-{(2Z)-2-[(1-ethyl-5-methoxy-2-methyl-1H-indol-3-yl)methylene]-3-oxo-2,3-dihydro-1-benzofuran-5-yl}-3-{4-[(1-methylpiperidin-4-yl)carbonyl]phenyl}urea;
1-(4-{[2-(dimethylamino)ethyl](methyl)amino}phenyl)-3-{(2Z)-2-[(1-ethyl-5-methoxy-2-methyl-1H-indol-3-yl)methylene]-3-oxo-2,3-dihydro-1-benzofuran-5-yl}urea;
1-(4-{[3-(dimethylamino)propyl](methyl)amino}phenyl)-3-{(2Z)-2-[(1-ethyl-5-methoxy-2-methyl-1H-indol-3-yl)methylene]-3-oxo-2,3-dihydro-1-benzofuran-5-yl}urea;
1-{4-[3-(dimethylamino)propoxy]phenyl}-3-{(2Z)-2-[(1-ethyl-5-methoxy-2-methyl-1H-indol-3-yl)methylene]-3-oxo-2,3-dihydro-1-benzofuran-5-yl}urea;
N-[2-(dimethylamino)ethyl]-4-[({(2Z)-2-[(1-ethyl-5-methoxy-1H-indol-3-yl)methylene]-3-oxo-2,3-dihydro-1-benzofuran-5-yl}carbamoyl)amino]-N-methylbenzamide;
1-[(2Z)-2-{[5-methoxy-1-(2-piperazin-1-ylethyl)-2-(1,3,5-trimethyl-1H-pyrazol-4-yl)-1H-indol-3-yl]methylene}-3-oxo-2,3-dihydro-1-benzofuran-5-yl]-3-methylurea;
N-[3-(dimethylamino)propyl]-4-[({(2Z)-2-[(1-ethyl-5-methoxy-2-methyl-1H-indol-3-yl)methylene]-3-oxo-2,3-dihydro-1-benzofuran-5-yl}carbamoyl)amino]-N-methylbenzamide;
1-{(2Z)-2-[(5-methoxy-1,2-dimethyl-1H-indol-3-yl)methylidene]-3-oxo-2,3-dihydro-1-benzofuran-5-yl}-3-{4-[(4-methylpiperazin-1-yl)carbonyl]phenyl}urea;
4-[({(2Z)-2-[(1-ethyl-5-methoxy-2-methyl-1H-indol-3-yl)methylene]-3-oxo-2,3-dihydro-1-benzofuran-5-yl}carbamoyl)amino]-N-[2-(methylamino)ethyl]benzamide;
4-[({(2Z)-2-[(2-cyclohexyl-5-methoxy-1H-indol-3-yl)methylene]-3-oxo-2,3-dihydro-1-benzofuran-5-yl}carbamoyl)amino]-N-[2-(methylamino)ethyl]benzamide;
4-[({(2Z)-2-[(5-methoxy-1,2-dimethyl-1H-indol-3-yl)methylene]-3-oxo-2,3-dihydro-1-benzofuran-5-yl}carbamoyl)amino]-N-[2-(methylamino)ethyl]benzamide;
1-[(2Z)-2-({5-methoxy-2-methyl-1-[2-(4-methylpiperazin-1-yl)ethyl]-1H-indol-3-yl}methylidene)-7-methyl-3-oxo-2,3-dihydro-1-benzofuran-5-yl]-3-methylurea;
1-{4-[4-(dimethylamino)butyl]phenyl}-3-{(2Z)-2-[(1-ethyl-5-methoxy-2-methyl-1H-indol-3-yl)methylidene]-3-oxo-2,3-dihydro-1-benzofuran-5-yl}urea;
1-[(2Z)-2-{[1-(2-{[2-(dimethylamino)ethyl]amino}ethyl)-5-methoxy-2-(1,3,5-trimethyl-1H-pyrazol-4-yl)-1H-indol-3-yl]methylidene}-3-oxo-2,3-dihydro-1-benzofuran-5-yl]-3-methylurea;
1-[(2Z)-2-{[1-(2-{[2-(dimethylamino)ethyl](methyl)amino}ethyl)-5-methoxy-2-(1,3,5-trimethyl-1H-pyrazol-4-yl)-1H-indol-3-yl]methylidene}-3-oxo-2,3-dihydro-1-benzofuran-5-yl]-3-methylurea;
N-[3-(dimethylamino)propyl]-4-[({(2Z)-2-[(1-ethyl-5-methoxy-2-methyl-1H-indol-3-yl)methylidene]-3-oxo-2,3-dihydro-1-benzofuran-5-yl}carbamoyl)amino]benzamide;
1-{4-[2-(dimethylamino)ethoxy]phenyl}-3-{(2Z)-2-[(1-ethyl-5-methoxy-2-methyl-1H-indol-3-yl)methylidene]-3-oxo-2,3-dihydro-1-benzofuran-5-yl}urea;
1-{4-[4-(dimethylamino)butoxy]phenyl}-3-{(2Z)-2-[(1-ethyl-5-methoxy-2-methyl-1H-indol-3-yl)methylidene]-3-oxo-2,3-dihydro-1-benzofuran-5-yl}urea;
4-({[(2Z)-2-({5-methoxy-2-methyl-1-[2-(4-methylpiperazin-1-yl)ethyl]-1H-indol-3-yl}methylidene)-3-oxo-2,3-dihydro-1-benzofuran-5-yl]carbamoyl}amino)-N-[2-(methylamino)ethyl]benzamide;
1-{4-[2-(dimethylamino)ethyl]phenyl}-3-{(2Z)-2-[(1-ethyl-5-methoxy-2-methyl-1H-indol-3-yl)methylidene]-3-oxo-2,3-dihydro-1-benzofuran-5-yl}urea;
1-{4-[3-(dimethylamino)propyl]phenyl}-3-{(2Z)-2-[(1-ethyl-5-methoxy-2-methyl-1H-indol-3-yl)methylidene]-3-oxo-2,3-dihydro-1-benzofuran-5-yl}urea;
1-[(2Z)-2-({5-methoxy-1-[2-(methylamino)ethyl]-2-(1,3,5-trimethyl-1H-pyrazol-4-yl)-1H-indol-3-yl}methylidene)-3-oxo-2,3-dihydro-1-benzofuran-5-yl]-3-methylurea;
1-[(2Z)-2-({1-[2-(dimethylamino)ethyl]-5-methoxy-2-(1,3,5-trimethyl-1H-pyrazol-4-yl)-1H-indol-3-yl}methylidene)-3-oxo-2,3-dihydro-1-benzofuran-5-yl]-3-methylurea;
1-{4-[4-(dimethylamino)butoxy]phenyl}-3-[(2Z)-2-({5-methoxy-2-methyl-1-[2-(4-methylpiperazin-1-yl)ethyl]-1H-indol-3-yl}methylidene)-3-oxo-2,3-dihydro-1-benzofuran-5-yl]urea;
1-(4-{[4-(dimethylamino)butyl](methyl)amino}phenyl)-3-{(2Z)-2-[(1-ethyl-5-methoxy-2-methyl-1H-indol-3-yl)methylidene]-3-oxo-2,3-dihydro-1-benzofuran-5-yl}urea;
N-{4-[({(2Z)-2-[(1-ethyl-5-methoxy-2-methyl-1H-indol-3-yl)methylidene]-3-oxo-2,3-dihydro-1-benzofuran-5-yl}carbamoyl)amino]phenyl}-N3,N3-dimethyl-b-alaninamide;
1-{(2Z)-2-[(1-ethyl-5-methoxy-2-methyl-1H-indol-3-yl)methylidene]-3-oxo-2,3-dihydro-1-benzofuran-5-yl}-3-{4-[(4-methylpiperazin-1-yl)sulfonyl]phenyl}urea;
1-(4-{[2-(dimethylamino)ethyl]amino}phenyl)-3-{(2Z)-2-[(1-ethyl-5-methoxy-2-methyl-1H-indol-3-yl)methylidene]-3-oxo-2,3-dihydro-1-benzofuran-5-yl}urea;
N-[2-(dimethylamino)ethyl]-4-[({(2Z)-2-[(1-ethyl-5-methoxy-2-methyl-1H-indol-3-yl)methylidene]-3-oxo-2,3-dihydro-1-benzofuran-5-yl}carbamoyl)amino]-N-methylbenzenesulfonamide;
1-[(2Z)-2-{[5-(2-methoxyethoxy)-2-(1,3,5-trimethyl-1H-pyrazol-4-yl)-1H-indol-3-yl]methylidene}-3-oxo-2,3-dihydro-1-benzofuran-5-yl]-3-methylurea;
1-[4-(dimethylamino)phenyl]-3-{(2Z)-2-[(1-ethyl-5-methoxy-2-methyl-1H-indol-3-yl)methylidene]-3-oxo-2,3-dihydro-1-benzofuran-5-yl}urea;
N-[2-(dimethylamino)ethyl]-4-({[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]carbamoyl}amino)-N-methylbenzamide;
1-{(2Z)-2-[(1-ethyl-5-methoxy-2-methyl-1H-indol-3-yl)methylidene]-3-oxo-2,3-dihydro-1-benzofuran-5-yl}-3-{4-[3-(methylamino)propoxy]phenyl}urea;
1-(4-{[2-(dimethylamino)ethyl]amino}phenyl)-3-[(2Z)-2-({5-methoxy-2-methyl-1-[2-(4-methylpiperazin-1-yl)ethyl]-1H-indol-3-yl}methylidene)-3-oxo-2,3-dihydro-1-benzofuran-5-yl]urea;
N-[4-({[(2Z)-2-({5-methoxy-2-methyl-1-[2-(4-methylpiperazin-1-yl)ethyl]-1H-indol-3-yl}methylidene)-3-oxo-2,3-dihydro-1-benzofuran-5-yl]carbamoyl}amino)phenyl]-N3,N3-dimethyl-b-alaninamide;
1-{(2Z)-2-[(1-ethyl-5-methoxy-2-methyl-1H-indol-3-yl)methylidene]-3-oxo-2,3-dihydro-1-benzofuran-5-yl}-3-[4-(morpholin-4-ylcarbonyl)phenyl]urea;
1-[(2Z)-2-({5-methoxy-2-methyl-1-[2-(4-methylpiperazin-1-yl)ethyl]-1H-indol-3-yl}methylidene)-3-oxo-2,3-dihydro-1-benzofuran-5-yl]-3-[4-(morpholin-4-ylcarbonyl)phenyl]urea;
4-[({(2Z)-2-[(2-cyclohexyl-5-methoxy-1H-indol-3-yl)methylidene]-3-oxo-2,3-dihydro-1-benzofuran-5-yl}carbamoyl)amino]-N-[2-(dimethylamino)ethyl]-N-methylbenzamide;
1-[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]-3-{4-[(4-methylpiperazin-1-yl)carbonyl]phenyl}urea;
1-[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]-3-[4-(morpholin-4-ylcarbonyl)phenyl]urea;
N-[4-({[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]carbamoyl}amino)phenyl]-N3,N3-dimethyl-b-alaninamide;
1-{(2Z)-2-[(2-bromo-5-methoxy-1H-indol-3-yl)methylidene]-3-oxo-2,3-dihydro-1-benzofuran-5-yl}-3-methylurea;
1-[(2Z)-2-{[7-fluoro-5-methoxy-2-(1,3,5-trimethyl-1H-pyrazol-4-yl)-1H-indol-3-yl]methylidene}-3-oxo-2,3-dihydro-1-benzofuran-5-yl]-3-methylurea;
N-{4-[({(2Z)-2-[(1-ethyl-5-methoxy-2-methyl-1H-indol-3-yl)methylidene]-3-oxo-2,3-dihydro-1-benzofuran-5-yl}carbamoyl)amino]phenyl}-N,N3,N3-trimethyl-b-alaninamide;
N-[4-({[(2Z)-2-({5-methoxy-2-methyl-1-[2-(4-methylpiperazin-1-yl)ethyl]-1H-indol-3-yl}methylidene)-3-oxo-2,3-dihydro-1-benzofuran-5-yl]carbamoyl}amino)phenyl]-N,N3,N3-trimethyl-b-alaninamide;
N-(4-{[(2-{[5-methoxy-2-(1,3,5-trimethyl-1H-pyrazol-4-yl)-1H-indol-3-yl]methylidene}-3-oxo-2,3-dihydro-1-benzofuran-5-yl)carbamoyl]amino}phenyl)-N,N3,N3-trimethyl-b-alaninamide;
1-(4-{[3-(dimethylamino)pyrrolidin-1-yl]carbonyl}phenyl)-3-[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]urea;
4-({[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]carbamoyl}amino)-N-methyl-N-(1-methylpyrrolidin-3-yl)benzamide;
1-{4-[(4-ethylpiperazin-1-yl)carbonyl]phenyl}-3-[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]urea;
1-[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]-3-(4-{[4-(1-methylethyl)piperazin-1-yl]carbonyl}phenyl)urea;
4-({[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]carbamoyl}amino)-N-methyl-N-(2-pyrrolidin-1-ylethyl)benzamide;
1-(4-{[4-(dimethylamino)piperidin-1-yl]carbonyl}phenyl)-3-[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]urea;
1-{4-[(3,4-dimethylpiperazin-1-yl)carbonyl]phenyl}-3-[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]urea;
4-({[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]carbamoyl}amino)-N,N-dimethylbenzamide;
1-{(2Z)-2-[(2-{1-[2-(dimethylamino)ethyl]-3,5-dimethyl-1H-pyrazol-4-yl}-5-methoxy-1H-indol-3-yl)methylidene]-3-oxo-2,3-dihydro-1-benzofuran-5-yl}-3-methylurea;
1-[(2Z)-2-({5-methoxy-2-[1-(2-methylpropyl)-1H-pyrazol-4-yl]-1H-indol-3-yl}methylidene)-3-oxo-2,3-dihydro-1-benzofuran-5-yl]-3-methylurea;
N-[2-(dimethylamino)ethyl]-3-({[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]carbamoyl}amino)-N-methylbenzamide;
N-[3-(dimethylamino)propyl]-3-({[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]carbamoyl}amino)-N-methylbenzamide;
1-[(2Z)-2-({5-methoxy-2-[1-(2-methoxyethyl)-3,5-dimethyl-1H-pyrazol-4-yl]-1H-indol-3-yl}methylidene)-3-oxo-2,3-dihydro-1-benzofuran-5-yl]-3-methylurea;
N-[2-(dimethylamino)ethyl]-4-({[(2Z)-2-{[7-fluoro-5-methoxy-2-(1,3,5-trimethyl-1H-pyrazol-4-yl)-1H-indol-3-yl]methylidene}-3-oxo-2,3-dihydro-1-benzofuran-5-yl]carbamoyl}amino)-N-methylbenzamide;
N-[2-(dimethylamino)ethyl]-4-({[(2Z)-2-{[5-methoxy-2-(1,3,5-trimethyl-1H-pyrazol-4-yl)-1H-indol-3-yl]methylidene}-3-oxo-2,3-dihydro-1-benzofuran-5-yl]carbamoyl}amino)benzamide;
1-[(2Z)-2-{[6-fluoro-5,7-dimethoxy-2-(1,3,5-trimethyl-1H-pyrazol-4-yl)-1H-indol-3-yl]methylidene}-3-oxo-2,3-dihydro-1-benzofuran-5-yl]-3-methylurea;
1-[(2Z)-2-{[6,7-difluoro-5-methoxy-2-(1,3,5-trimethyl-1H-pyrazol-4-yl)-1H-indol-3-yl]methylidene}-3-oxo-2,3-dihydro-1-benzofuran-5-yl]-3-methylurea;
1-[(2Z)-2-{[2-(3,5-dimethyl-1H-pyrazol-4-yl)-7-fluoro-5-methoxy-1H-indol-3-yl]methylidene}-3-oxo-2,3-dihydro-1-benzofuran-5-yl]-3-methylurea;
1-(2-aminoethyl)-3-[(2Z)-2-{[7-fluoro-5-methoxy-2-(1,3,5-trimethyl-1H-pyrazol-4-yl)-1H-indol-3-yl]methylidene}-3-oxo-2,3-dihydro-1-benzofuran-5-yl]urea;
1-[2-(dimethylamino)ethyl]-3-[(2Z)-2-{[7-fluoro-5-methoxy-2-(1,3,5-trimethyl-1H-pyrazol-4-yl)-1H-indol-3-yl]methylidene}-3-oxo-2,3-dihydro-1-benzofuran-5-yl]urea;
1-[(2Z)-2-({7-fluoro-5-methoxy-2-[1-(2-methylpropyl)-1H-pyrazol-4-yl]-1H-indol-3-yl}methylidene)-3-oxo-2,3-dihydro-1-benzofuran-5-yl]-3-methylurea;
1-[(2Z)-2-{[7-fluoro-5-methoxy-2-(1-methyl-1H-pyrazol-4-yl)-1H-indol-3-yl]methylidene}-3-oxo-2,3-dihydro-1-benzofuran-5-yl]-3-methylurea;
1-{4-[(dimethylamino)methyl]phenyl}-3-{(2Z)-2-[(1-ethyl-5-methoxy-2-methyl-1H-indol-3-yl)methylidene]-3-oxo-2,3-dihydro-1-benzofuran-5-yl}urea;
1-{4-[(1,1-dioxidothiomorpholin-4-yl)carbonyl]phenyl-3-[(2Z)-2-[5-methoxy-2-(1,3,5-trimethyl-1H-pyrazol-4-yl)-1H-indol-3-yl]methylidene}-3-oxo-2,3-dihydro-1-benzofuran-5-yl]urea;
1-[(2Z)-2-{[7-fluoro-5-methoxy-2-(1,3,5-trimethyl-1H-pyrazol-4-yl)-1H-indol-3-yl]methylidene}-3-oxo-2,3-dihydro-1-benzofuran-5-yl]-3-(4-{[4-(2-hydroxyethyl)piperazin-1-yl]carbonyl}phenyl)urea;
1-[(2Z)-2-{[2-(1,3-dimethyl-1H-pyrazol-4-yl)-5-methoxy-1H-indol-3-yl]methylidene}-3-oxo-2,3-dihydro-1-benzofuran-5-yl]-3-methylurea;
1-[(2Z)-2-{[2-(1,5-dimethyl-1H-pyrazol-4-yl)-5-methoxy-1H-indol-3-yl]methylidene}-3-oxo-2,3-dihydro-1-benzofuran-5-yl]-3-methylurea;
1-[(2Z)-2-({5-methoxy-2-[1-methyl-4-(trifluoromethyl)-1H-pyrazol-3-yl]-1H-indol-3-yl}methylidene)-3-oxo-2,3-dihydro-1-benzofuran-5-yl]-3-methylurea;
1-[(2Z)-2-{[5-methoxy-7-(trifluoromethyl)-2-(1,3,5-trimethyl-1H-pyrazol-4-yl)-1H-indol-3-yl]methylidene}-3-oxo-2,3-dihydro-1-benzofuran-5-yl]-3-methylurea;
1-[(2Z)-2-{[5-methoxy-7-methyl-2-(1,3,5-trimethyl-1H-pyrazol-4-yl)-1H-indol-3-yl]methylidene}-3-oxo-2,3-dihydro-1-benzofuran-5-yl]-3-methylurea;
1-[(2Z)-2-{[7-fluoro-5-methoxy-2-(1,3,5-trimethyl-1H-pyrazol-4-yl)-1H-indol-3-yl]methylidene}-3-oxo-2,3-dihydro-1-benzofuran-5-yl]-3-(2-pyrrolidin-1-ylethyl)urea;
1-[(2Z)-2-{[7-fluoro-5-methoxy-2-(1,3,5-trimethyl-1H-pyrazol-4-yl)-1H-indol-3-yl]methylidene}-3-oxo-2,3-dihydro-1-benzofuran-5-yl]-3-[2-(methylamino)ethyl]urea;
1-(4-{[4-(dimethylamino)piperidin-1-yl]carbonyl}phenyl)-3-[(2Z)-2-{[7-fluoro-5-methoxy-2-(1,3,5-trimethyl-1H-pyrazol-4-yl)-1H-indol-3-yl]methylidene}-3-oxo-2,3-dihydro-1-benzofuran-5-yl]urea;
1-{4-[(3,4-dimethylpiperazin-1-yl)carbonyl]phenyl}-3-[(2Z)-2-{[7-fluoro-5-methoxy-2-(1,3,5-trimethyl-1H-pyrazol-4-yl)-1H-indol-3-yl]methylidene}-3-oxo-2,3-dihydro-1-benzofuran-5-yl]urea;
1-[(2Z)-2-{[7-fluoro-5-methoxy-2-(1,3,5-trimethyl-1H-pyrazol-4-yl)-1H-indol-3-yl]methylidene}-3-oxo-2,3-dihydro-1-benzofuran-5-yl]-3-[4-(morpholin-4-ylcarbonyl)phenyl]urea;
1-(4-{[3-(dimethylamino)pyrrolidin-1-yl]carbonyl}phenyl)-3-[(2Z)-2-{[7-fluoro-5-methoxy-2-(1,3,5-trimethyl-1H-pyrazol-4-yl)-1H-indol-3-yl]methylidene}-3-oxo-2,3-dihydro-1-benzofuran-5-yl]urea; and
N-[2-(dimethylamino)ethyl]-4-({[(2Z)-2-{[7-fluoro-5-methoxy-2-(1,3,5-trimethyl-1H-pyrazol-4-yl)-1H-indol-3-yl]methylidene}-3-oxo-2,3-dihydro-1-benzofuran-5-yl]carbamoyl}amino)benzamide;

18) A composition comprising a compound of claim 1 and a pharmaceutically acceptable carrier.

19) The composition of claim 17, wherein the pharmaceutically acceptable carrier is suitable for oral administration and the composition comprises an oral dosage form.

20) A composition comprising a compound of claim 1; a second compound selected from the group consisting of a topoisomerase I inhibitor, a MEK1/2 inhibitor, a HSP90 inhibitor, procarbazine, dacarbazine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epirubicin, 5-fluorouracil, docetaxel, paclitaxel, leucovorin, levamisole, irinotecan, estramustine, etoposide, nitrogen mustards, BCNU, carmustine, lomustine, vinblastine, vincristine, vinorelbine, cisplatin, carboplatin, oxaliplatin, imatinib mesylate, Avastin (bevacizumab), hexamethylmelamine, topotecan, tyrosine kinase inhibitors, tyrphostins, herbimycin A, genistein, erbstatin, hydroxyzine, glatiramer acetate, interferon beta-1a, interferon beta-1b, natalizumab, and lavendustin A; and a pharmaceutically acceptable carrier.

21) The composition of claim 19, wherein the second compound is Avastin.

22) A method of treating a PI3K-related disorder, comprising administering to a mammal in need thereof a compound of claim 1 in an amount effective to treat a PI3K-related disorder.

23) The method of claim 21, wherein the PI3K-related disorder is selected from restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, and cancer.

24) The method of claim 22, wherein the PI3K-related disorder is cancer.

25) The method of claim 23, wherein the cancer is selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer.

26) A method of treating an mTOR-related disorder, comprising administering to a mammal in need thereof a compound of claim 1 in an amount effective to treat an mTOR-related disorder.

27) The method of claim 25, wherein the mTOR-related disorder is selected from restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, and cancer.

28) The method of claim 26, wherein the mTOR-related disorder is cancer.

29) The method of claim 27, wherein the cancer is selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer.

30) A method of treating advanced renal cell carcinoma, comprising administering to a mammal in need thereof a compound of claim 1 in an amount effective to treat advanced renal cell carcinoma.

31) A method of treating acute lymphoblastic leukemia, comprising administering to a mammal in need thereof a compound of claim 1 in an amount effective to treat acute lymphoblastic leukemia.

32) A method of treating acute malignant melanoma, comprising administering to a mammal in need thereof a compound of claim 1 in an amount effective to treat malignant melanoma.

33) A method of treating soft-tissue or bone sarcoma, comprising administering to a mammal in need thereof a compound of claim 1 in an amount effective to treat soft-tissue or bone sarcoma.

34) A method of treating a cancer selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer comprising administering to a mammal in need thereof the composition of claim 20 in an amount effective to treat the cancer.

35) A method of inhibiting mTOR in a subject, comprising administering to a subject in need thereof a compound of claim 1 in an amount effective to inhibit mTOR.

36) A method of inhibiting PI3K in a subject, comprising administering to a subject in need thereof a compound of claim 1 in an amount effective to inhibit PI3K.

37) A method of inhibiting mTOR, PI3K, and hSMG-1 together in a subject, comprising administering to a subject in need thereof a compound of claim 1 in an amount effective to inhibit mTOR, PI3K, and hSMG-1.

38) A method of synthesizing a compound of claim 1 comprising:
a) condensing a compound of the formula CXI with a compound of formula CXII: 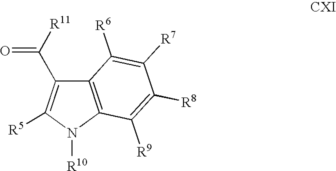
under acidic conditions, and A and R¹-R¹¹ are as defined above in claim 1 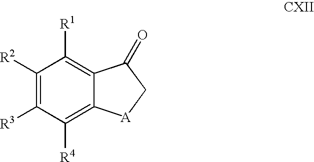
thereby producing a compound of formula I′: 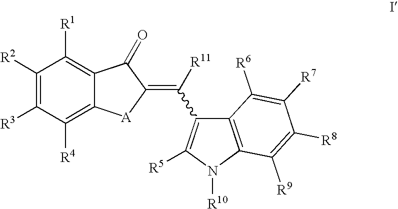
b) optionally reducing the compound of formula I′ and thereby producing a compound of formula I″: 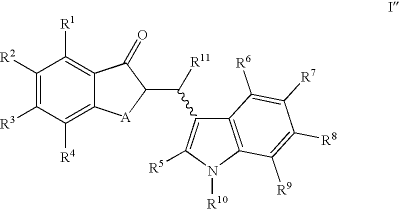
or a pharmaceutically acceptable salt thereof.

39) The method of claim 37 in which the compound of formula CXI is prepared by a process comprising:
a) acylation with R¹¹C(O)X, wherein X is halogen, or Vilsmeier-Haack formylation, of a compound of formula CIX: 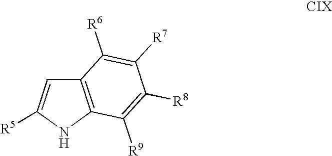
thereby producing a compound of formula CXI wherein R¹⁰ is H, having the formula CX: 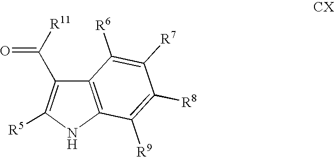
b) optionally alkylating the compound of formula CX with R¹⁰Cl, wherein R¹⁰ is as defined in claim 1 excepting H thereby producing a corresponding compound of Formula CXI.
